(19) United States
(12) Patent Application Publication
(10) Pub. No.: US 2022/0127568 A1
(43) Pub. Date: Apr. 28, 2022

(54) COMPOSITIONS AND METHODS FOR GENERATING HAIR CELLS BY INHIBITING EPIGENETIC TARGETS

(71) Applicant: Frequency Therapeutics, Inc., Lexington, MA (US)

(72) Inventors: Will MCLEAN, North Haven, CT (US); Megan HARRISON, Middletown, CT (US); Melissa HILL-DRZEWI, Durham, CT (US); Bradley TAIT, North Andover, MA (US)

(73) Assignee: Frequency Therapeutics, Inc., Lexington, MA (US)

(21) Appl. No.: 17/429,274

(22) PCT Filed: Feb. 7, 2020
(86) PCT No.: PCT/US2020/017354

§ 371 Date: Aug. 6, 2021

Related U.S. Application Data
(60) Provisional application No. 62/803,347, filed on Feb. 8, 2019.
(60) Provisional application No. 62/803,351, filed on Feb. 8, 2019.
(60) Provisional application No. 62/803,352, filed on Feb. 8, 2019.
(60) Provisional application No. 62/803,353, filed on Feb. 8, 2019.

(51) Int. Cl. C12N 5/071; C12N 5/0797; A61K 45/06; A61P 27/16

(52) U.S. Cl. CPC C12N 5/0628; C12N 5/0623; A61K 45/06; C12N 2500/30; C12N 2501/415; C12N 2501/065; C12N 2501/72; A61P 27/16

(57) ABSTRACT

Provided are compositions and methods comprising an epigenetic agent and a Wnt agonist for increasing proliferation of cochlear supporting cells or vestibular supporting cells, and related methods of treating inner ear hearing or balance disorders.

### CROSS-REFERENCE TO RELATED APPLICATIONS

This application claims priority to U.S. Provisional Application No. 62/803,347 filed Feb. 8, 2019, entitled “COMPOSITIONS AND METHODS FOR GENERATING HAIR CELLS BY INHIBITING LSD1”; U.S. Provisional Application No. 62/803,351 filed Feb. 8, 2019, entitled “COMPOSITIONS AND METHODS FOR GENERATING HAIR CELLS BY INHIBITING EZH2”; U.S. Provisional Application No. 62/803,352 filed Feb. 8, 2019, entitled “COMPOSITIONS AND METHODS FOR GENERATING HAIR CELLS BY INHIBITING DOT1L”; and U.S. Provisional Application No. 62/803,353, filed Feb. 8, 2019, entitled “COMPOSITIONS AND METHODS FOR GENERATING HAIR CELLS BY INHIBITING KDM”, the disclosures of which are incorporated by reference herein.

### FIELD OF THE INVENTION

The present disclosure relates to compositions and methods comprising an epigenetic agent and a Wnt agonist for increasing proliferation of cochlear supporting cells or vestibular supporting cells, production of an expanded population of cochlear or vestibular cells, in particular Lgr5+ cells, and related methods of treating an inner hearing or balance disorder, in particular sensorineural hearing loss.

### BACKGROUND OF THE INVENTION

Generation of sensory hair cells from undifferentiated cell populations is likely to provide a therapy for several inner ear hearing and balance disorders that arise from damage and loss of sensory hair cells in the inner ear. Replacement hair cells could be produced in situ, in the damaged sensory epithelium of the inner ear, or grown in vitro and then delivered to the inner ear, and so strategies for generation of sensory cells in vitro and in vivo are of interest.

Sensorineural hearing loss (SNHL), which is largely due to the loss of sensory hair cells and their neural connections is a widespread problem. It is estimated that over one billion young people are at risk for noise-related sensorineural hearing loss. SNHL accounts for about 90% of all hearing loss (Li et al., Adv. Drug Deliv. Rev. 108, 2-12, 2017), and leading causes include advanced age, ototoxic medications, and noise exposure (Liberman & Kujawa, Hear. Res. 349, 138-147, 2017). The majority of children and adults with SNHL are managed with hearing aids or cochlear implants, as there is currently no therapeutic option to restore function in the damaged inner ear (see, for example, Ramakers et al., Laryngoscope 125, 2584-92, 2015; Raman et al., Effectiveness of Cochlear Implants in Adults with Sensorineural Hearing Loss. Agency for Healthcare Research and Quality (US), 2011; and Roche & Hansen, Otolaryngol. Clin. North Am. 48, 1097-116, 2015). Loss or damage of hair cells in the vestibular system of inner ear can lead to balance disorders (for example, dizziness and vertigo), incidences of which also increase with age. Like the cochlea, there is currently no therapeutic option to restore function in damaged vestibular epithelia, and regeneration of hair cells may also be an effective therapeutic approach for balance disorders.

The underlying pathophysiologic changes of sensory epithelia of the inner ear in patients with inner ear hearing loss or balance disorders includes damage and loss of sensory transducers of the cochlear and vestibular systems called hair cells. Hair cells are susceptible to damage, and although other species such as birds, fish, and amphibians can regenerate these cells throughout life, mammals lack this ability (Fujioka et al., Trends Neurosci, 38, 139-44, 2015).

Several approaches are being investigated to replace damaged or absent hair cells in mammalian inner ear sensory epithelia (reviewed in Mittal et al. Front Mol Neurosci. (2017); 10: 236). These include cell-based approaches (which aim to deliver exogenous cells to the inner ear to restore the sensory epithelia) and gene-based approaches (which aim to deliver exogenous genes to the sensory epithelia and reprogram endogenous cells to generate hair cells). For example, adenovirus-mediated delivery of Atoh1 is able to stimulate cells within the sensory epithelia to differentiate into hair cells (Izumikawa et al. Nat Med. 2005 March; 11(3):271-6. Epub 2005 Feb. 13). One drawback with these approaches is the requirement to deliver cells or vectors into the inner of the patient, which can be challenging in the complex system of the inner ear. Molecular approaches, in which the endogenous signaling pathways of inner ear cells are modulated by exogenous agents are therefore attractive, as the delivery of such agents for prolonged periods of time is likely to be more straightforward than cell-based or gene-based approaches.

Using molecular agents to initiate transdifferentiation, in which existing supporting cells of the cochlear are stimulated to differentiate into replacement hair cells, is one area of interest. However, transdifferentiation alone (i.e. without proliferation) may not provide sufficient hair cells to regenerate a functioning cochlea or vestibular system, especially as an associated depletion of the supporting cell population could also negatively impact the functioning of the cochlea or vestibular organs. Focus has therefore been placed on activation of proliferative response in the supporting cells, in order to provide a new population of cells that could differentiate into hair cells, thereby replacing lost or damaged hair cells.

A subset of supporting cells that express Lgr5 have been shown to be endogenous hair cell progenitors with stimulation via the Wnt/beta-catenin pathway leading to proliferation and differentiation of these cells into sensory hair cells (Bramhall et al. 2014 Stem Cell Reports 2, 311-322). More recently, a combination of a Wnt pathway agonist (a GSK3β inhibitor) in combination with a histone deacetylase complex (HDAC) inhibitor has been found to stimulate expansion of an Lgr5+ supporting cell population in the inner ear and regenerate hair cells ex vivo (McLean et al. Cell Rep. 2017 Feb. 21; 18(8): 1917-1929).

There remains a need for the development of effective hair cell regeneration strategies in the inner ear, in vivo, which may include boosting the proliferation of supporting cells of sensory epithelium of the inner ear beyond that which has been achieved previously.

### SUMMARY OF THE INVENTION

In various aspects the disclosure provides methods for increasing proliferation of a cochlear supporting cell or a vestibular supporting cell, by contacting the supporting cell with a first epigenetic agent that is (a) a lysine specific demethylase 1 (LSD1) inhibitor, an enhancer of zeste homolog 2 (EZH2) inhibitor, a disruptor of telomeric silencing 1-like (DOT1L) inhibitor, or a histone lysine demethylase (KDM) inhibitor; and (b) a Wnt agonist. In some embodiments, (a) and (b) can occur in any order or simultaneously.

In other aspects the disclosure provides method for producing an expanded population of cochlear or vestibular cells, by contacting the supporting cell with a first epigenetic agent that is (a) a lysine specific demethylase 1 (LSD1) inhibitor, an enhancer of zeste homolog 2 (EZH2) inhibitor, a disruptor of telomeric silencing 1-like (DOT1L) inhibitor, or a histone lysine demethylase (KDM) inhibitor and (b) a Wnt agonist. In some embodiments. (a) and (b) can occur in any order or simultaneously.

The cochlear supporting cell(s) or vestibular supporting cell(s) express(es) leucine-rich repeat-containing G-protein coupled receptor 5 (Lgr5). The cochlear supporting cell(s) or vestibular supporting cell(s) are/is a mature cell(s). The expanded population of cochlear or vestibular cells expresses leucine-rich repeat-containing G-protein coupled receptor 5 (Lgr5).

The epigenetic agent in combination with the Wnt agonist increases the Lgr5 Activity of the expanded population of cochlear or vestibular cells by a factor of at least 10, 20, 30, 40, 50, 75, 100 or 200% compared to a Wnt agonist alone or a Wnt agonist in combination with valproic acid, wherein the Lgr5 Activity is measured in a Stem Cell Proliferation Assay

In further aspects the disclosure provides methods of treating a subject who has, or is at risk of, developing an inner ear hearing or balance disorder, by administering to the subject with a first epigenetic agent that is (a) a lysine specific demethylase 1 (LSD1) inhibitor, an enhancer of zeste homolog 2 (EZH2) inhibitor, a disruptor of telomeric silencing 1-like (DOT1L) inhibitor, or a histone lysine demethylase (KDM) inhibitor and (b) a Wnt agonist. In some embodiments, (a) and (b) can occur in any order or simultaneously.

In some embodiments, the inner ear hearing or balance disorder is sensorineural hearing loss. The treatment results in improved auditory function when assessed by behavioral audiometry or auditory brainstem response (ABR) testing or any other measure of hearing loss as defined herein.

In some embodiments, the LSD1 inhibitor is irreversible. The LSD1 inhibitor can be, for example, GSK-2879552, GSK-LSD1, Tranylcypromine, Phenelzine sulfate, RN-1, or ORY-1001. In some embodiments, the LSD1 inhibitor is selected from the group consisting of GSK-2879552 or Tranylcypromine

In some embodiments, the GSK2879552 is at a concentration of about between 4 nM to 30 μM.

In some embodiments, the GSK-LSD1 is at a concentration of about between 4 nM to 50 μM.

In some embodiments, the Tranylcypromine is at a concentration of about between 0.1 μM to 20 μM.

In some embodiments, the Phenelzine sulfate at a concentration of about between 0.1 μM to 10 μM.

In some embodiments, the RN-1 at a concentration of about between 1 nM to 1000 nM.

In some embodiments, the ORY-1001 at a concentration of about between 1 nM to 1000 nM.

In some embodiments, the EZH2 inhibitor is an enzymatic inhibitor.

In some embodiments, the EZH2 inhibitor is selected from the group consisting of: CPI-1205, CPI-169, E11, PE-06821497, tazemetostat, valemetostat, CPI-360, EPZ011989, UNC 2399, and PF 06726304.

In some embodiments, the CPI-1205 is at a concentration of about between 10 nM to 1000 nM.

In some embodiments, the CPI-169 is at a concentration of about between 1 μM to 10 μM.

In some embodiments, the E11 is at a concentration of about between 1 μM to 10 μM.

In some embodiments, the PF-06821497 is at a concentration of about between 1 nM to 100 nM.

In some embodiments, the tazemetostat is at a concentration of about between 0.1 μM to 1.5 μM.

In some embodiments, the valemetostat is at a concentration of about between 10 nM to 1000 nM.

In some embodiments, the CPI-360 is at a concentration of about between 100 nM to 100 μM.

In some embodiments, the EPZ011989 is at a concentration of about between 10 nM to 10 μM.

In some embodiments, the UNC 2399 is at a concentration of about between 1 μM to 1000 μM.

In some embodiments, the PF-06726304 is at a concentration of about between 10 nM to 10 μM.

In some embodiments, the DOT1L inhibitor is a S-adenosyl methionine (SAM) competitive inhibitor.

In some embodiments, the DOT1L inhibitor is selected from the group consisting of EPZ004777, pinometostat and SGC0946.

In some embodiments, the EPZ004777 is at a concentration of about between 0.5 μM to 45 μM.

In some embodiments, the pinometostat is at a concentration of about between 0.1 μM to 10 μM.

In some embodiments, the SGC0946 is at a concentration of about between 0.5 μM to 5 μM.

In some embodiments, the KDM inhibitor is AS 8351,TC-E 5002 or EPT 103182.

In some embodiments, the AS 8351 is at a concentration of about between 0.5 μM to 5 μM.

In some embodiments, the TC-E 5002 is at a concentration of about between 0.1 μM to 10 μM.

In some embodiments, the EPT-103182 is at a concentration of about 1 nM to 100 nM.

The Wnt agonist is for example, a GSK3 inhibitor. The GSK3 inhibitor is AZD1080, UY2090314, a substituted 3-Imidazo[1,2-a]pyridin-3-yl-4-(1,2,3,4-tetrahydro-[1,4]diazepino-[6,7,1-hi]indol-7-yl)pyrrole-2,5-dione, GSK3 inhibitor XXII or CHIR99021.

In some embodiments, the GSK3 inhibitor is CHIR99021.

In some embodiments, the AZD1080 is at a concentration of about between 0.5 μM to 5 μM.

In some embodiments, the LY2090314 is at a concentration of about between 4 nM to 40 nM.

In some embodiments, the substituted 3-Imidazo[1,2-a]pyridin-3-yl-4-(1,2,3,4-tetrahydro-[1,4]diazepino-[6,7,1-hi]indol-7-yl)pyrrole-2,5-dione is at a concentration of about between 5 nM to 500 nM.

In some embodiments, the GSK3 inhibitor XXII at a concentration of about between 0.1 μM to 1 μM.

In some embodiments, the CHIR99021 is at a concentration of about between 1 μM to 10 μM.

In various embodiments the methods of the invention further include contacting the cochlear or vestibular supporting cell(s) with, or administering to the subject, an epigenetic agent. In some embodiments, the second epigenetic agent is an HDAC inhibitor, an LSD1 inhibitor, an EZH2 inhibitor, a DOT1L inhibitor, or a KDM inhibitor.

In some embodiments, the HDAC inhibitor is for example, Valproic Acid (VPA). The VPA is at a concentration of about between 100 μM to 4,000 μM.

The LSD1 inhibitor can be, for example, GSK-2879552, GSK-LSD1, Tranylcypromine, Phenelzine sulfate, RN-1, or ORY-1001.

The GSK2879552 can be, for example, at a concentration of about between 4 nM to 30 μM.

The GSK-LSD1 can be, for example, at a concentration of about between 4 nM to 50 μM.

The Tranylcypromine can be, for example, at a concentration of about between 0.1 μM to 20 μM.

The Phenelzine sulfate can be, for example, at a concentration of about between 0.1 μM to 10 μM.

In some embodiments, the RN-1 can be, for example, at a concentration of about between 1 nM to 1000 nM.

In some embodiments, the ORY-1001 can be, for example, at a concentration of about between 1 nM to 1000 nM.

In some embodiments, the EZH2 inhibitor is an enzymatic inhibitor. The EZH2 inhibitor can be, for example, CPI-1205, CPI-169, E11, PF-06821497, tazemetostat, valemetostat, CPI-360, EPZ011989, or UNC 2399.

In some embodiments, the CPI-1205 is at a concentration of about between 10 nM to 1000 nM.

In some embodiments, the E11 is at a concentration of about between 1 μM to 10 μM.

In some embodiments, the PF-06821497 is at a concentration of about between 1 nM to 100 nM.

In some embodiments, the tazemetostat is at a concentration of about between 0.1 μM to 1.5 μM.

In some embodiments, the valemetostat is at a concentration of about between 10 nM to 1000 nM.

In some embodiments, the CPI-169 is at a concentration of about between 1 μM to 10 μM.

In some embodiments, the CPI-360 is at a concentration of about between 1 nM to 100 μM.

In some embodiments, the EPZ011989 is at a concentration of about between 10 nM to 10 μM.

In some embodiments, the UNC 2399 is at a concentration of about between 1 μM to 1000 μM.

In some embodiments, the PF-06726304 is at a concentration of about between 10 nM to 10 μM.

In some embodiments, the DOT1L inhibitor is an S-adenosyl methionine (SAM) competitive inhibitor. The DOT1L inhibitor is for example EPZ004777, pinometostat or SGC0946.

In some embodiments, the EPZ004777 is at a concentration of about between 0.5 μM to 45 μM.

In some embodiments, the pinometostat is at a concentration of about between 0.1 μM to 10 μM.

In some embodiments, the the SGC0946 is at a concentration of about between 0.5 μM to 5 μM.

In some embodiments, the KDM inhibitor is for example, AS 8351, EPT 103182, or TC-E 5002.

In some embodiments, the AS 8351 is at a concentration of about between 0.5 μM to 5 μM.

In some embodiments, the TC-E 5002 is at a concentration of about between 0.1 μM to 10 μM.

In some embodiments, the EPT103182 is at a concentration of about between 1 nM to 100 nM

In some embodiments of the methods of the disclosure, the LSD1 inhibitor, EZH2 inhibitor, DOT1L inhibitor, or KDM inhibitor is administered locally and/or systemically. The Wnt agonist is administered locally and/or systemically. The second epigenetic agent is administered locally and/or systemically. In some embodiments, the local administration is to the tympanic membrane, the middle ear or the inner ear. In some embodiments, the systemic administration is oral or parenteral.

In some embodiments, the LSD1 inhibitor is GSK2879552 and is administered locally at a dose of 4 nM. In some embodiments, the LSD1 inhibitor is tranylcypromine and is administered locally at a dose of 4 μM. In some embodiments, the LSD1 inhibitor is GSK2879552 and is administered systemically at a unit dose of 1 mg. In some embodiments, the LSD1 inhibitor is tranylcypromine and is administered systemically at a unit dose of 15 mg. In some embodiments, the Wnt agonist is CHIR99021 and is administered locally at a dose of 4 μM. In some embodiments, the the second epigenetic agent is valproic acid (VPA) and is administered locally at a dose of 1 mM.

In some embodiments, the second epigenetic agent is valproic acid (VPA) and is administered systemically at a unit dose of 500 mg.

In additional aspects the disclosure provides pharmaceutical compositions containing a first epigenetic agent that is a LSD1 inhibitor, a Wnt agonist and a pharmaceutically acceptable carrier. In some embodiments, the LSD1 inhibitor is irreversible. In some embodiments, the LSD1 inhibitor is selected from the group consisting of GSK-2879552, GSK-LSD1, Tranylcypromine, Phenelzine sulfate, RN-1, or ORY-1001.

In some embodiments, the GSK2879552 is at a concentration of about between 4 μM to 30 mM.

In some embodiments, the GSK-LSD1 is at a concentration of about between 4 μM to 50 mM.

In some embodiments, the Tranylcypromine is at a concentration of about between 0.1 mM to 20 mM.

In some embodiments, the Phenelzine sulfate is at a concentration of about between 0.1 mM to 10 mM.

In some embodiments, the ORY-1001 is at a concentration of about between 1 μM to 1000 μM.

In some embodiments, the RN-1 is at a concentration of about between 1 μM to 1000 μM.

In some embodiments, wherein the EZH2 inhibitor is CPI-1205 and is administered locally at a dose of about 0.01 μM to 100 μM or about 1 μM.

In some embodiments, the EZH2 inhibitor is CPI-169 and is administered locally at a dose of about 0.01 μM to 100 μM or about 1 μM.

In some embodiments, the EZH2 inhibitor is E11 and is administered locally at a dose of about 0.05 μM to 500 μM or about 5 μM.

In some embodiments, the EZH2 inhibitor is Tazemetostat and is administered locally at a dose of about 0.003 μM to 30 μM or about 0.3 μM.

In some embodiments, the EZH2 inhibitor is CPI-1205 and is administered systemically at a unit dose of about 8 mg to 1200 mg or about 800 mg.

In some embodiments, the EZH2 inhibitor is CPI-169 and is administered systemically at a unit dose of about 1 mg to 500 mg or about 100 mg.

In some embodiments, the EZH2 inhibitor is E11 and is administered systemically at a unit dose of about 1 mg to 500 mg or about 100 mg.

In some embodiments, the EZH2 inhibitor is Tazemetostat and is administered systemically at a unit dose of about 1 mg to 500 mg or about 100 mg.

In some embodiments, the Wnt agonist is CHIR99021 and is administered locally at a dose of about 0.04 μM to 400 μM or about 4 μM.

In some embodiments, the second epigenetic agent is valproic acid (VPA) and is administered locally at a dose of about 0.01 mM to 100 mM or about 1 mM.

In some embodiments, the second epigenetic agent is valproic acid (VPA) and is administered systemically at a unit dose of about 5 mg to 1000 mg or about 500 mg.

In some embodiments, the DOT1L inhibitor is EPZ004777, pinometostat or SGC0946.

In some embodiments, the DOT1L inhibitor is EPZ004777 and is administered systemically at a dose of 50 mg.

In some embodiments, the DOT1L inhibitor is EPZ004777 and is administered locally at a dose of 15 μM.

In some embodiments, the DOT1L inhibitor is pinometostat and is administered systemically at a dose of 60 mg.

In some embodiments, the DOT1L inhibitor is pinometostat and is administered locally at a dose of 10 μM.

In some embodiments, the DOT1L inhibitor is SGC0946 and is administered systemically at a dose of 50 mg.

In some embodiments, the DOT1L inhibitor is SGC0946 and is administered locally at a dose of 1.7 μM.

In some embodiments, the KDM inhibitor is AS 8351 and is administered locally at a dose of 2 μM.

In some embodiments, the KDM inhibitor is TC-E 5002 and is administered locally at a dose of 400 nm.

In some embodiments, the KDM inhibitor is AS 8351 and is administered systemically at a unit dose of 100 mg.

In some embodiments, the KDM inhibitor is TC-E 5002 and is administered systemically at a unit dose of 100 mg.

In some embodiments, the Wnt agonist is a GSK3 inhibitor. The GSK3 inhibitor is AZD1080, LY2090314, a substituted 3-Imidazo[1,2-a]pyridin-3-yl-4-(1,2,3,4-tetrahydro-[1,4]diazepino-[6,7,1-hi]indol-7-yl)pyrrole-2,5-dione, GSK3 inhibitor XXII or CHIR99021.

In some embodiments, the wherein AZD1080 is at a concentration of about between 0.5 mM to 5 mM.

In some embodiments, the LY2090314 is at a concentration of about between 4 μM to 40 μM.

In some embodiments, the substituted 3-Imidazo[1,2-a]pyridin-3-yl-4-(1,2,3,4-tetrahydro-[1,4]diazepino-[6,7,1-hi]indol-7-yl)pyrrole-2,5-dione is at a concentration of about between 5 μM to 500 μM.

In some embodiments, the GSK3 inhibitor XXII at a concentration of about between 0.1 mM to 1 mM.

In some embodiments, the CHIR99021 is at a concentration of about between 1 mM to 10 mM.

In some embodiments, the pharmaceutical compositions further contain a second epigenetic agent. In some embodiments, the second epigenetic agent is an HDAC inhibitor, a LSD1 inhibitor, an EZH2 inhibitor, a DOT1L inhibitor, or a KDM inhibitor.

In some embodiments, the HDAC inhibitor is Valproic Acid (VPA).

In some embodiments, the VPA is at a concentration of about between 100 mM to 4,000 mM.

In some embodiments, the LSD1 inhibitor is selected from the group consisting of GSK-2879552, GSK-LSD1, Tranylcypromine, Phenelzine sulfate, RN-1 or ORY-1001.

In some embodiments, the GSK2879552 is at a concentration of about between 4 μM to 30 mM.

In some embodiments, the GSK-LSD1 is at a concentration of about between 4 μM to 50 mM.

In some embodiments, the Tranylcypromine is at a concentration of about between 0.1 mM to 20 mM.

In some embodiments, the Phenelzine sulfate is at a concentration of about between 0.1 mM to 10 mM.

In some embodiments, the ORY-1001 is at a concentration of about between 1 μM to 1000 μM.

In some embodiments, the RN-1 is at a concentration of about between 1 μM to 1000 μM.

In some embodiments, the EZH2 inhibitor is an enzymatic inhibitor. In some embodiments, the EZH2 inhibitor for example CPI-1205, CPI-169, E11, PF-06821497, tazemetostat, valemetostat, CPI-360, EPZ011989, UNC 2399, or PF-06726304.

In some embodiments, the CPI-1205 is at a concentration of about between 10 μM to 1000 μM.

In some embodiments, the CPI-169 is at a concentration of about between 1 mM to 10 mM.

In some embodiments, the E11 is at a concentration of about between 1 mM to 10 mM.

In some embodiments, the PF-06821497 is at a concentration of about between 1 μM to 100 μM.

In some embodiments, the tazemetostat is at a concentration of about between 0.1 mM to 10 mM.

In some embodiments, the valemetostat is at a concentration of about between 10 μM to 1000 μM.

In some embodiments, the CPI-360 is at a concentration of about between 100 μM to 1000 mM.

In some embodiments, the EPZ011989 is at a concentration of about between 10 μM to 10 mM.

In some embodiments, the UNC 2399 is at a concentration of about between 1 mM to 1000 mM.

In some embodiments, the PF-06726304 is at a concentration of about between 10 μM to 10 mM.

In some embodiments, the DOT1L inhibitor is an S-adenosyl methionine (SAM) competitive inhibitor. The DOT1L inhibitor is for example, EPZ004777, pinometostat and SGC0946.

In some embodiments, the EPZ004777 is at a concentration of about between 0.5 mM to 45 mM.

In some embodiments, the pinometostat is at a concentration of about between 0.1 mM to 10 mM.

In some embodiments, the SGC0946 is at a concentration of about between 0.5 mM to 5 mM.

In some embodiments, the KDM inhibitor is for example AS 8351, EPT103182 or TC-E 5002.

In some embodiments, the AS 8351 is at a concentration of about between 0.5 mM to 5 mM.

In some embodiments, the EPT103182 is at a concentration of about between 1 μM to 100 μM.

In some embodiments, the TC-E 5002 is at a concentration of about between 1 mM to 10 mM.

In some embodiments, the various embodiments the pharmaceutical composition is in a biocompatible matrix.

In some embodiments, the biocompatible matrix includes hyaluronic acid, hyaluronates, lecithin gels, pluronics, poly(ethyleneglycol), poloxamers, chitosans, xyloglucans, collagens, fibrins, polyesters, poly(lactides), poly(glycolide), polylactic-co-glycolic acid (PLGA), sucrose acetate isobutyrate, glycerol monooleate, poly anhydrides, poly caprolactone sucrose, glycerol monooleate, silk materials, or a combination thereof.

In some embodiments, the pharmaceutical compositions are formulated for local or systemic administration

In some embodiments, the pharmaceutical compositions are used in treating or preventing an inner ear hearing or balance disorder. In some embodiments, the inner ear hearing is sensorineural hearing loss.

In some embodiments, the pharmaceutical compositions are used in treating or preventing an inner ear hearing or in the manufacture of a medicament for the treatment or prevention of an inner ear hearing or balance disorder. In some embodiments, the inner ear hearing is sensorineural hearing loss.

Embodiments of the invention also include a lysine specific demethylase 1 (LSD1) inhibitor, an enhancer of zeste homolog 2 (EZH2) inhibitor, a disruptor of telomeric silencing 1-like (DOT1L) inhibitor, or a histone lysine demethylase (KDM) inhibitor for use in treating or preventing an inner ear hearing or balance disorder in a subject, where the subject has been, or will be, administered a Wnt agonist.

Embodiments of the invention also include a Wnt agonist for use in treating or preventing an inner ear hearing or balance disorder in a subject, where the subject has been, or will be, administered a lysine specific demethylase 1 (LSD1) inhibitor, an enhancer of zeste homolog 2 (EZH2) inhibitor, a disruptor of telomeric silencing 1-like (DOT1L) inhibitor, or a histone lysine demethylase (KDM) inhibitor.

Embodiments of the invention also include a epigenetic agent for use in treating or preventing an inner ear hearing or balance disorder in a subject, where the subject has been, or will be, administered a lysine specific demethylase 1 (LSD1) inhibitor, an enhancer of zeste homolog 2 (EZH2) inhibitor, a disruptor of telomeric silencing 1-like (DOT1L) inhibitor, or a histone lysine demethylase (KDM) inhibitor, and a Wnt agonist.

In some embodiments, the inner ear hearing or balance disorder is sensorineural hearing loss.

Embodiments of the invention also include container comprising a lysine specific demethylase 1 (LSD1) inhibitor, an enhancer of zeste homolog 2 (EZH2) inhibitor, a disruptor of telomeric silencing 1-like (DOT1L) inhibitor, or a histone lysine demethylase (KDM) inhibitor and instructions, Where those instructions describe the inhibitor's use for treating or preventing an inner ear hearing or balance disorder in a subject, where the instructions require that the subject has been, or will be, administered a Wnt agonist.

Embodiments of the invention also include container comprising a Wnt agonist and instructions, where those instructions describe the Wnt agonist's use in treating or preventing an inner ear hearing or balance disorder in a subject, where the instructions require that the subject has been, or will be, administered a lysine specific demethylase 1 (LSD1) inhibitor, an enhancer of zeste homolog 2 (EZH2) inhibitor, a disruptor of telomeric silencing 1-like (DOT1L) inhibitor, or a histone lysine demethylase (KDM) inhibitor.

Embodiments of the invention also include container comprising an epigenetic agent and instructions, where those instructions describe the epigenetic agent's use in treating or preventing an inner ear hearing or balance disorder in a subject, where the instructions require that the subject has been, or will be, administered a lysine specific demethylase 1 (LSD1) inhibitor, an enhancer of zeste homolog 2 (EZH2) inhibitor, a disruptor of telomeric silencing 1-like (DOT1L) inhibitor, or a histone lysine demethylase (KDM) inhibitor and a Wnt agonist. In some embodiments, the inner ear hearing disorder is sensorineural hearing loss.

Unless otherwise defined, all technical and scientific terms used herein have the same meaning as commonly understood by one of ordinary skill in the art to which this disclosure belongs. In the specification, the singular forms also include the plural unless the context clearly dictates otherwise. Although methods and materials similar or equivalent to those described herein can be used in the practice or testing of the present invention, suitable methods and materials are described below. In the case of conflict, the present specification, including definitions, will control. In addition, the materials, methods and examples are illustrative only and are not intended to be limiting.

Other features and advantages of the invention will be apparent from the following detailed description and claims.

### BRIEF DESCRIPTION OF THE DRAWINGS

FIG. 1A is a graph depicting that the LSD1 inhibitor tranylcypromine does not proliferate Lgr5 GFP+ cochlear progenitor cells in a background of growth factors compared to cells grown in a background of growth factors plus CHIR99021 (CHIR; EFI-C) or CHIR and VPA (EFI-CV). The y-axis depicts Lgr5 GFP(+) cell count and the x-axis depicts concentration of tranylcypromine. Media components include 50 ng/mL EG, 50 ng/mL bFGF, 50 ng/mL IGR1, 4 μM CHIR99021, 1 mM VPA and 0-10 μM tranylcypromine.

FIG. 1B is a graph depicting that the LSD1 inhibitor tranylcypromine does not enrich for Lgr5 GFP+ cochlear progenitor cells in a background of growth factors compared to cells grown in a background of growth factors plus CHIR (EFI-C) or CHIR and VPA (EFI-CV). The y-axis depicts Lgr5 GFP(+) cell proliferation percentage and the x-axis depicts concentration of tranylcypromine. Media components include 50 ng/mL EG, 50 ng/mL bFGF, 50 ng/mL IGR1, 4 μM CHIR99021, 1 mM VPA and 0-10 μM tranylcypromine.

FIG. 2A is a graph depicting that the LSD1 inhibitor tranylcypromine enhances Lgr5 GFP+ progenitor cell proliferation when combined with CHIR in a background of growth factors. The y-axis depicts Lgr5 GFP(+) cell count and the x-axis depicts control conditions (background growth factors plus CHIR (EFI-C) or CHIR and VPA (EFI-CV)) versus CHIR+tranylcypromine (EFI-C-TRANYL). Media components include 50 ng/mL EG, 50 ng/mL bFGF, 50 ng/mL IGR1, 4 μM CHIR99021, 1 mM VPA and 2 μM tranylcypromine.

FIG. 2B is a graph depicting that the LSD1 inhibitor tranylcypromine enhances enrichment of Lgr5 GFP+ cochlear progenitor cells when combined with CHIR in a background of growth factors. The y-axis depicts Lgr5 GFP(+) cell proliferation percentage and the x-axis depicts control conditions (EFI-C) or (EFI-CV) versus CHIR+tranylcypromine (EFI-C-TRANYL). Media components include 50 ng/mL EG, 50 ng/mL bFGF, 50 ng/mL IGR1, 4 μM CHIR99021, 1 mM VPA and 2 μM tranylcypromine.

FIG. 3A is a graph depicting that the LSD1 inhibitor tranylcypromine enhances Lgr5 GFP+ progenitor cell proliferation when combined with CHIR and VPA in a background of growth factors. The y-axis depicts Lgr5 GFP(+) cell count and the x-axis depicts control conditions (EFI-CV) versus CHIR+VPA+tranylcypromine (EFI-CV-TRANYL). Media components include 50 ng/mL EG, 50 ng/mL bFGF, 50 ng/mL IGR1, 4 μM CHIR99021, 1 mM VPA, and 4 μM tranylcypromine.

FIG. 3B is a graph depicting that the LSD1 inhibitor tranylcypromine enhances enrichment of Lgr5 GFP+ cochlear progenitor cells when combined with CHIR and VPA in a background of growth factors. The y-axis depicts Lgr5 GFP(+) cell proliferation percentage and the x-axis depicts control conditions (EFI-CV) versus CHIR+VPA+tranylcypromine (EFI-CV-TRANYL). Media components include 50 ng/mL EG, 50 ng/mL bFGF, 50 ng/mL IGR1, 4 μM CHIR99021, 1 mM VPA, and 4 μM tranylcypromine.

FIG. 4A is a graph depicting that the LSD1 inhibitor GSK2879552 enhances Lgr5 GFP+ progenitor cell proliferation when combined with CHIR and VPA in a background of growth factors. The y-axis depicts Lgr5 GFP(+) cell count and the x-axis depicts control conditions (EFI-C) or (EFI-CV) versus CHIR+VPA+GSK2879552 (EFI-CV-GS). Media components include 50 ng/mL EG, 50 ng/mL bFGF, 50 ng/mL IGR1, 4 μM CHIR99021, 1 mM VPA, and 370 nM GSK2879552.

FIG. 4B is a graph depicting that the LSD1 inhibitor GSK2879552 enhances enrichment of Lgr5 GFP+ cochlear progenitor cells when combined with CHIR and VPA in a background of growth factors. The y-axis depicts Lgr5 GFP(+) cell proliferation percentage and the x-axis depicts control conditions (EFI-C) or (EFI-CV) versus CHIR+VPA+GSK2879552 (EFI-CV-GS). Media components include 50 ng/mL EG, 50 ng/mL bFGF, 50 ng/mL IGR1, 4 μM CHIR99021, 1 mM VPA, and 370 nM GSK2879552.

FIG. 5A is a graph depicting that the LSD1 inhibitor GSK-LSD1 enhances Lgr5 GFP+ progenitor cell proliferation when combined with CHIR and VPA in a background of growth factors. The y-axis depicts Lgr5 GFP(+) cell count and the x-axis depicts control conditions (EFI-C) or (EFI-CV) versus CHIR+VPA+GSK-LSD1 (EFI-CV-GS). Media components include 50 ng/mL EG, 50 ng/mL bFGF, 50 ng/mL IGR1, 4 μM CHIR99021, 1 mM VPA, and 4.5 nM GSK-LSD1.

FIG. 5B is a graph depicting that the LSD1 inhibitor GSK-LSD1 enhances enrichment of Lgr5 GFP+ cochlear progenitor cells when combined with CHIR and VPA in a background of growth factors. The y-axis depicts Lgr5 GFP(+) cell proliferation percentage and the x-axis depicts control conditions (EFI-C) or (EFI-CV) versus CHIR+VPA+GSK-LSD1 (EFI-CV-GS). Media components include 50 ng/mL EG, 50 ng/mL bFGF, 50 ng/mL IGR1, 4 μM CHIR99021, 1 mM VPA, and 4.5 nM GSK-LSD1.

FIG. 6A is a graph depicting that the LSD1 inhibitor ORY-1001 does not proliferate Lgr5 GFP+ cochlear progenitor cells in a background of growth factors compared to cells grown in a background of growth factors plus CHIR99021 (CHIR; EFI-C) or CHIR and VPA (EFI-CV). The y-axis depicts Lgr5 GFP(+) cell area and the x-axis depicts concentration of ORY-1001. Media components include 50 ng/mL EG, 50 ng/mL bFGF, 50 ng/mL IGR1, 4 μM CHIR99021, 1 mM VPA and 0-30 μM ORY-1001.

FIG. 6B is a graph depicting that the LSD1 inhibitor ORY-1001 does not enrich for Lgr5 GFP+ cochlear progenitor cells in a background of growth factors compared to cells grown in a background of growth factors plus CHIR (EFI-C) or CHIR and VPA (EFI-CV). The y-axis depicts Lgr5 GFP(+) cell area percentage and the x-axis depicts concentration of ORY-1001. Media components include 50 ng/mL EG, 50 ng/mL bFGF, 50 ng/mL IGR1, 4 μM CHIR99021, 1 mM VPA and 0-30 μM ORY-1001.

FIG. 7A is a graph depicting that the LSD1 inhibitor ORY-1001 further enhances Lgr5 GFP+ progenitor cell proliferation when combined with CHIR+VPA in a background of growth factors growth factors compared to CHIR+VPA. The y-axis depicts Lgr5 GFP(+) cell area and the x-axis depicts media components: E=50 ng/mL EGF, F=50 ng/mL bFGF, I=50 ng/mL IGR1, C=CHIR=4 uM CHIR99021, V=VPA=1 mM Valproic Acid Sodium Salt, T=TRANYL=7 μM tranylcypromine, ORY=41 nM ORY-1001.

FIG. 7B is a graph depicting that LSD1 inhibitor ORY-1001 further enhances enrichment of Lgr5 GFP+ cochlear progenitor cells when combined with CHIR+VPA in a background of growth factors growth factors compared to CHIR+VPA. The y-axis depicts Lgr5 GFP(+) cell area and the x-axis depicts media components: E=50 ng/mL EGF, F=50 ng/mL bFGF, I=50 ng/mL IGR1, C=CHIR=4 uM CHIR99021, V=VPA=1 mM Valproic Acid Sodium Salt, T=TRANYL=7 uM tranylcypromine, ORY=41 nM ORY-1001.

FIG. 8A is a graph depicting that the EZH2 inhibitor EPZ6438 does not proliferate Lgr5 GFP+ cochlear progenitor cells in a background of growth factors compared to cells grown in a background of growth factors plus CHIR99021 (EFI-C) or CHIR+VPA (EFI-CV). The y-axis depicts Lgr5 GFP(+) cell count and the x-axis depicts concentration of EPZ6438. Media components include 50 ng/mL EG, 50 ng/mL bFGF, 50 ng/mL IGR1, 4 μM CHIR99021, 1 mM VPA, and 0-10 μM EPZ6438.

FIG. 8B is a graph depicting that the EZH2 inhibitor EPZ6438 does not enrich for Lgr5 GFP+ cochlear progenitor cells in a background of growth factors compared to cells grown in a background of growth factors plus CHIR (EFI-C) or CHIR+VPA (EFI-CV). The y-axis depicts Lgr5 GFP(+) cell proliferation percentage and the x-axis depicts concentration of EPZ6438. Media components include 50 ng/mL EG, 50 ng/mL bFGF, 50 ng/mL IGR1, 4 μM CHIR99021, 1 mM VPA and 0-10 μM EPZ6438.

FIG. 9A is a graph depicting that the EZH2 inhibitor EL1 enhances Lgr5 GFP+ progenitor cell proliferation when combined with CHIR in a background of growth factors. The y-axis depicts Lgr5 GFP(+) cell count and the x-axis depicts control conditions (background growth factors plus CHIR (EFI-C) or plus CHIR+VPA(EFI-CV)) versus CHIR+EL1 (EFI-C-EL1). Media components include 50 ng/mL EG, 50 ng/mL bFGF, 50 ng/mL IGR1, 4 μM CHIR99021, 1 mM VPA and 5 μM EL1.

FIG. 9B is a graph depicting that the EZH2 inhibitor EL1 enhances enrichment of Lgr5 GFP+ cochlear progenitor cells when combined with CHIR in a background of growth factors. The y-axis depicts Lgr5 GFP(+) cell proliferation percentage and the x-axis depicts control conditions (EFI-C) and (EFI-CV) versus CHIR+EL1 (EFI-C-EL1). Media components include 50 ng/mL EG, 50 ng/mL, bFGF, 50 ng/mL IGR1, 4 μM CHIR99021 and 5 μM EL1.

FIG. 10A is a graph depicting that the EZH2 inhibitor EPZ6438 enhances Lgr5 GFP+ progenitor cell proliferation when combined with CHIR in a background of growth factors. The y-axis depicts Lgr5 GFP(+) cell count and the x-axis depicts control conditions (EFI-C) and (EFI-CV) versus CHIR+EPZ6438 (EFI-C-EPZ). Media components include 50 ng/mL, EG, 50 ng/mL, bFGF, 50 ng/mL IGR1, 4 μM CHIR99021, 1 mM VPA and 0.37 μM EPZ6438.

FIG. 10B is a graph depicting that the EZH2 inhibitor EPZ6438 enhances enrichment of Lgr5 GFP+ cochlear progenitor cells when combined with CHIR in a background of growth factors. The y-axis depicts Lgr5 GFP(+) cell proliferation percentage and the x-axis depicts control conditions (EFI-C) and (EFI-CV) versus CHIR+EPZ6438 (EFI-C-EPZ). Media components include 50 ng/mL, EG, 50 ng/mL bFGF, 50 ng/mL IGR1, 4 μM CHIR99021, 1 mM VPA and 0.37 μM EPZ6438.

FIG. 11A is a graph depicting that the EZH2 inhibitor CPI-169 enhances Lgr5 GFP+ progenitor cell proliferation when combined with CHIR in a background of growth factors. The y-axis depicts Lgr5 GFP(+) cell count and the x-axis depicts control conditions (background growth factors plus CHIR (EFI-C) or plus CHIR+VPA(EFI-CV)) versus CHIR+CPI-169 (EFI-C-CPI). Media components include 50 ng/mL EG, 50 ng/mL bFGF, 50 ng/mL IGR1, 4 μM CHIR99021 and 1.11 μM CPI-169.

FIG. 11B is a graph depicting that the EZH2 inhibitor CPI-169 enhances enrichment of Lgr5 GFP+ cochlear progenitor cells when combined with CHIR in a background of growth factors. The y-axis depicts Lgr5 GFP(+) cell proliferation percentage and the x-axis depicts control conditions (EFI-C) and (EFI-CV) versus CHIR+CPI-169 (EFI-C-CPI-169). Media components include 50 ng/mL EG, 50 ng/mL bFGF, 50 ng/mL IGR1, 4 μM CHIR99021, 1 μM VPA and 1.11 μM CPI-169.

FIG. 12A is a graph depicting that the EZH2 inhibitor CPI-360 does not proliferate Lgr5 GFP+ cochlear progenitor cells in a background of growth factors. The y-axis depicts Lgr5 GFP(+) cell area and the x-axis depicts concentration of CPI-360. Media components include 50 ng/mL EGF, 50 ng/mL bFGF, 50 ng/mL IGR1, 4 μM CHIR99021, 1 mM VPA and 0-30 μM CPI-360.

FIG. 12B is a graph depicting that the EZH2 inhibitor CPI-360 does not enrich for Lgr5 GFP+ cochlear progenitor cells in a background of growth factors. The y-axis depicts Lgr5 GFP(+) cell area percentage and the x-axis depicts concentration of CPI-360. Media components include 50 ng/mL EGF, 50 ng/mL bFGF, 50 ng/mL IGR1, 4 μM CHIR99021, 1 mM VPA and 0-30 μM CPI-360.

FIG. 13A is a graph depicting that the EZH2 inhibitor CPI-360 enhances Lgr5 GFP+ progenitor cell proliferation when combined with CHIR in a background of growth factors compared to CHIR, alone. The y-axis depicts Lgr5 GFP(+) cell area and the x-axis depicts media conditions: E=50 ng/mL EGF, F=50 ng/mL bFGF, I=50 ng/mL IGR1, C=CHIR=4 μM CHIR99021, CPI-360=3.33 μM CPI-360.

FIG. 13B is a graph depicting that the EZH2 inhibitor CPI-360 enhances enrichment of Lgr5 GFP+ cochlear progenitor cells when combined with CHIR in a background of growth factors compared to CHIR alone. The y-axis depicts Lgr5 GFP(+) cell area percentage and the x-axis depicts media conditions: E=50 ng/mL EGF, F=50 ng/mL bFGF, I=50 ng/mL IGR1, C=CHIR=4 μM CHIR99021, CPI-360=3.33 μM CPI-360.

FIG. 14A is a graph depicting that the EZH2 inhibitor CPI-360 enhances Lgr5 GIP+ progenitor cell proliferation when combined with CHIR in a background of growth factors compared to CHIR alone. The y-axis depicts Lgr5 GFP(+) cell area and the x-axis depicts media conditions: E=50 ng/mL EGF, F=50 ng/mL bFGF, I=50 ng/mL IGR1, C=CHIR=4 μM CHIR99021, CPI-360=3.33 μM CPI-360.

FIG. 14B is a graph depicting that the EZH2 inhibitor CPI-360 enhances enrichment of Lgr5 GFP+ cochlear progenitor cells when combined with CHIR in a background of growth factors compared to CHIR alone. The y-axis depicts Lgr5 GFP(+) cell area percentage and the x-axis depicts media conditions: E=50 ng/mL EGF, F=50 ng/mL bFGF, I=50 ng/mL IGR1, C=CHIR=4 μM CHIR99021, CPI-360=3.33 μM CPI-360.

FIG. 15A is a graph depicting that the EZH2 inhibitor CPI-360 enhances Lgr5 GFP+ progenitor cell proliferation when combined with CHIR in a background of growth factors. The y-axis depicts Lgr5 GFP(+) cell area and the x-axis depicts media conditions: E=50 ng/mL EGF, F=50 ng/mL bFGF, I=50 ng/mL IGR1, C=CHIR=4 μM CHIR99021, V=VPA=1 mM Valproic Acid Sodium Salt, CPI-360=3.33 μM CPI-360.

FIG. 15B is a graph depicting that the EZH2 inhibitor CPI-360 enhances enrichment of Lgr5 GFP+ cochlear progenitor cells when combined with CHIR in a background of growth factors. The y-axis depicts Lgr5 GFP(+) cell area percentage and the x-axis depicts media conditions: E=50 ng/mL EGF, F=50 ng/mL bFGF, I=50 ng/mL IGR1, C=CHIR=4 μM CHIR99021, V=VPA=1 mM Valproic Acid Sodium Salt, CPI-360=3.33 μM CPI-360.

FIG. 16A is a graph depicting that the EZH2 inhibitor CPI-360 enhances Lgr5 GFP+ progenitor cell proliferation when combined with CHIR in a background of growth factors. The y-axis depicts Lgr5 GFP(+) cell area and the x-axis depicts media conditions: E=50 ng/mL EGF, F=50 ng/mL bFGF, I=50 ng/mL IGR1, C=CHIR=4 μM CHIR99021, V=VPA=1 mM Valproic Acid Sodium Salt, CPI-360=3.33 μM CPI-360.

FIG. 16B is a graph depicting that the EZH2 inhibitor CPI-360 enhances enrichment of Lgr5 GFP+ cochlear progenitor cells when combined with CHIR in a background of growth factors compared to CHIR alone. The y-axis depicts Lgr5 GFP(+) cell area percentage and the x-axis depicts media conditions: E=50 ng/mL EGF, F32 50 ng/mL bFGF, I=50 ng/mL IGR1, C=CHIR=4 μM CHIR99021, V=VPA=1 mM Valproic Acid Sodium Salt, CPI-360=3.33 μM CPI-360.

FIG. 17A is a graph depicting that the EZH2 inhibitor CPI-1205 does not proliferate Lgr5 GFP+ cochlear progenitor cells in a background of growth factors. The y-axis depicts Lgr5 GFP(+) cell area and the x-axis depicts concentration of CPI-1205. Media components include 50 ng/mL EGF, 50 ng/mL bFGF, 50 ng/mL IGR1, 4 μM CHIR99021, 1 mM VPA and 0-30 μM CPI-1205.

FIG. 17B is a graph depicting that the EZH2 inhibitor CPI-1205 does not enrich for Lgr5 GFP+ cochlear progenitor cells in a background of growth factors. The y-axis depicts Lgr5 GFP(+) cell area and the x-axis depicts concentration of CPI-1205. Media components include 50 ng/mL EGF, 50 ng/mL bFGF, 50 ng/mL IGR1, 4 μM CHIR99021, 1 mM VPA and 0-30 μM CPI-1205.

FIG. 18A is a graph depicting that the EZH2 inhibitor CPI-1205 enhances Lgr5 GFP+ progenitor cell proliferation when combined with CHIR in a background of growth factors compared to CHIR alone. The y-axis depicts Lgr5 GFP(+) cell area and the x-axis depicts media conditions: E=50 ng/mL EGF, F=50 ng/mL bFGF, I=50 ng/mL IGR1, C=CHIR=4 μM CHIR99021, CPI-1205=3.33 μM CPI-1205.

FIG. 18B is a graph depicting that the EZH2 inhibitor CPI-1205 enhances enrichment of Lgr5 GFP+ cochlear progenitor cells when combined with CHIR in a background of growth factors compared to CHIR alone. The y-axis depicts Lgr5 GFP(+) cell area percentage and the x-axis depicts media conditions: E=50 ng/mL EGF, F=50 ng/mL bFGF, I=50 ng/mL IGR1, C=CHIR=4 μM CHIR99021, CPI-1205=3.33 μM CPI-1205.

FIG. 19A is a graph depicting that the EZH2 inhibitor CPI-1205 enhances Lgr5 GFP+ progenitor cell proliferation when combined with CHIR in a background of growth factors compared to CHIR alone. The y-axis depicts Lgr5 GFP(+) cell area and the x-axis depicts media conditions: E=50 ng/mL EGF, F=50 ng/mL bFGF, I=50 ng/mL IGR1, C=CHIR=4 μM CHIR99021, V=VPA=1 mM Valproic Acid Sodium Salt, CPI-1205=3.33 μM CPI-1205.

FIG. 19B is a graph depicting that the EZH2 inhibitor CPI-1205 enhances enrichment of Lgr5 GFP+ cochlear progenitor cells when combined with CHIR in a background of growth factors compared to CHIR, alone. The y-axis depicts Lgr5 GFP(+) cell area percentage and the x-axis depicts media conditions: E=50 ng/mL EGF, F=50 ng/mL bFGF, I=50 ng/mL IGR1, C=CHIR=4 μM CHIR99021, V=VPA=1 mM Valproic Acid Sodium Salt, CPI-1205=3.33 μM CPI-1205.

FIG. 20A is a graph depicting that the EZH2 inhibitor PF 06726304 acetate does not proliferate Lgr5 GFP+ cochlear progenitor cells in a background of growth factors. The y-axis depicts Lgr5 GFP(+) cell area and the x-axis depicts concentration of PF 06726304 acetate. Media components include 50 ng/mL EGF, 50 ng/mL bFGF, 50 ng/mL IGR1, 4 μM CHIR99021, 1 mM VPA and 0-90 μM PF 06726304 acetate.

FIG. 20B is a graph depicting that the EZH2 inhibitor PF 06726304 acetate does not enrich for Lgr5 GFP+ cochlear progenitor cells in a background of growth factors. The y-axis depicts Lgr5 GFP(+) cell area and the x-axis depicts concentration of PF 06726304 acetate. Media components include 50 ng/mL EGF, 50 ng/mL bFGF, 50 ng/mL IGR1, 4 μM CHIR99021, 1 mM VPA and 0-90 μM PF 06726304 acetate.

FIG. 21A is a graph depicting that the EZH2 inhibitor PF 06726304 acetate enhances Lgr5 GFP+ progenitor cell proliferation when combined with CHIR in a background of growth factors compared to CHIR alone. The y-axis depicts Lgr5 GFP(+) cell area and the x-axis depicts media conditions: E=50 ng/mL EGF, F=50 ng/mL bFGF, I=50 ng/mL IGR1, C=CHIR=4 μM CHIR99021, PF0=370 nM PF 06726304 acetate.

FIG. 21B is a graph depicting that the EZH2 inhibitor PF 06726304 acetate enhances enrichment of Lgr5 GFP+ cochlear progenitor cells when combined with CHIR in a background of growth factors compared to CHIR alone. The y-axis depicts Lgr5 GFP(+) cell area percentage and the x-axis depicts media conditions: E=50 ng/mL EGF, F=50 ng/mL bFGF, I=50 ng/mL IGR1, C=CHIR→4 μM CHIR99021, PF0=370 nM PF 06726304 acetate.

FIG. 22A is a graph depicting that the EZH2 inhibitor PF 06726304 enhances Lgr5 GFP+ progenitor cell proliferation when combined with CHIR in a background of growth factors compared to CHIR alone. The y-axis depicts Lgr5 GFP(+) cell area and the x-axis depicts media conditions: E=50 ng/mL EGF, F=50 ng/mL bFGF, I=50 ng/mL IGR1, C=CHIR=4 μM CHIR99021, V=VPA=1 mM Valproic Acid Sodium Salt, PF0=370 nM PF 06726304 acetate.

FIG. 22B is a graph depicting that the EZH2 inhibitor PF 06726304 acetate enhances enrichment of Lgr5 GFP+ cochlear progenitor cells when combined with CHIR in a background of growth factors compared to CHIR alone. The y-axis depicts Lgr5 GFP(+) cell area percentage and the x-axis depicts media conditions: E=50 ng/mL EGF, F=50 ng/mL bFGF, I=50 ng/mL IGR1, C=CHIR=4 μM CHIR99021, V=VPA=1 mM Valproic Acid Sodium Salt, PF0=370 nM PF 06726304 acetate.

FIG. 23 is a graph depicting that the EZH2 inhibitor PF 06726304 enhances enrichment of Lgr5 GFP+ cochlear progenitor cells when combined with CHIR in a background of growth factors compared to CHIR alone. The y-axis depicts Lgr5 GFP(+) cell area percentage and the x-axis depicts media conditions: E=50 ng/mL EGF, F=50 ng/mL bFGF, I=50 ng/mL IGR1, C=CHIR=4 uM CHIR99021, V=VPA=1 mM Valproic Acid Sodium Salt, T=7 μM tranylcypromine, PF0=333.3 nM PF 06726304 acetate.

FIG. 24 is a graph depicting that the EZH2 inhibitor PF 06726304 enhances enrichment of Lgr5 GFP+ cochlear progenitor cells when combined with CHIR in a background of growth factors compared to CHIR+VPA. The y-axis depicts Lgr5 GFP(+) cell area percentage and the x-axis depicts media conditions: E=50 ng/mL EGF, F=50 ng/mL bFGF, I=50 ng/mL IGR1, C=CHIR=4 uM CHIR99021, V=VPA=1 mM Valproic Acid Sodium Salt, T=7 μM tranylcypromine, PF0=333.3 nM PF 06726304 acetate.

FIG. 25A is a graph depicting that the EZH2 inhibitor EPZ011989 does not proliferate Lgr5 GFP+ cochlear progenitor cells in a background of growth factors. The y-axis depicts Lgr5 GFP(+) cell area and the x-axis depicts concentration of EPZ011989. Media components include 50 ng/mL EGF, 50 ng/mL bFGF, 50 ng/mL IGR1, 4 μM CHIR99021, 1 mM VPA and 0-10 μM EPZ011989.

FIG. 25B is a graph depicting that the EZH2 inhibitor EPZ011989 does not enrich for Lgr5 GFP+ cochlear progenitor cells in a background of growth factors. The y-axis depicts Lgr5 GFP(+) cell area and the x-axis depicts concentration of EPZ011989. Media components include 50 ng/mL EGF, 50 ng/mL, bFGF, 50 ng/mL IGR1, 4 μM CHIR99021, 1 mM VPA and 0-10 μM EPZ011989.

FIG. 26A is a graph depicting that the EZH2 inhibitor EPZ011989 enhances Lgr5 GFP+ progenitor cell proliferation when combined with CHIR in a background of growth factors compared to CHIR alone. The y-axis depicts Lgr5 GFP(+) cell area and the x-axis depicts media conditions: E=50 ng/mL EGF, F=50 ng/mL bFGF, F=50 ng/mL IGR1, C=CHIR=4 uM CHIR99021, EPZ=123 nM EPZ011989.

FIG. 26B is a graph depicting that the EZH2 inhibitor EPZ011989 enhances enrichment of Lgr5 GFP+ cochlear progenitor cells when combined with CHIR in a background of growth factors compared to CHIR alone. The y-axis depicts Lgr5 GFP(+) cell area percentage and the x-axis depicts media conditions: E=50 ng/mL EGF, F=50 ng/mL bFGF, I=50 ng/mL IGR1, C=CHIR=4 uM CHIR99021, EPZ=123 nM EPZ011989.

FIG. 27A is a graph depicting that the EZH2 inhibitor EPZ011989 enhances Lgr5 GFP+ progenitor cell proliferation when combined with CHIR in a background of growth factors compared to CHIR alone. The y-axis depicts Lgr5 GFP(+) cell area and the x-axis depicts media conditions: E=50 ng/mL EGF, F=50 ng/mL bFGF, I=50 ng/mL IGR1, C=CHIR=4 uM CHIR99021, EPZ=123 nM EPZ011989.

FIG. 27B is a graph depicting that the EZH2 inhibitor EPZ011989 enhances enrichment of Lgr5 GFP+ cochlear progenitor cells when combined with CHIR in a background of growth factors compared to CHIR alone. The y-axis depicts Lgr5 GFP(+) cell area percentage and the x-axis depicts media conditions: E=50 ng/mL EGF, F=50 ng/mL bFGF, I=50 ng/mL IGR1, C=CHIR=4 uM CHIR99021, EPZ=123 nM EPZ011989.

FIG. 28A is a graph depicting that the EZH2 inhibitor EPZ011989 enhances Lgr5 GFP+ progenitor cell proliferation when combined with CHIR in a background of growth factors compared to CHIR alone. The y-axis depicts Lgr5 GFP(+) cell area and the x-axis depicts media conditions: E=50 ng/mL EGF, F=50 ng/mL bFGF, I=50 ng/mL IGR1, C=CHIR=4 uM CHIR99021, V=VPA=1 mM Valproic Acid Sodium Salt, EPZ=123 nM EPZ011989.

FIG. 28B is a graph depicting that the EZH2 inhibitor EPZ011989 enhances enrichment of Lgr5 GFP+ cochlear progenitor cells when combined with CHIR in a background of growth factors compared to CHIR alone. The y-axis depicts Lgr5 GFP(+) cell area percentage and the x-axis depicts media conditions: E=50 ng/mL EGF, F=50 ng/mL bFGF, I=50 ng/mL IGR1, C=CHIR=4 uM CHIR99021, V=VPA=1 mM Valproic Acid Sodium Salt, EPZ=123 nM EPZ011989.

FIG. 29A is a graph depicting that the EZH2 inhibitor EPZ011989 enhances Lgr5 GFP+ progenitor cell proliferation when combined with CHIR in a background of growth factors compared to CHIR alone. The y-axis depicts Lgr5 GFP(+) cell area and the x-axis depicts media conditions: E=50 ng/mL EGF, F=50 ng/mL bFGF, I=50 ng/mL IGR1, C=CHIR=4 uM CHIR99021, V=VPA=1 mM Valproic Acid Sodium Salt, EPZ=123 nM EPZ011989.

FIG. 29B is a graph depicting that the EZH2 inhibitor EPZ011989 enhances enrichment of Lgr5 GFP+ cochlear progenitor cells when combined with CHIR in a background of growth factors compared to CHIR alone. The y-axis depicts Lgr5 GFP(+) cell area percentage and the x-axis depicts media conditions: E=50 ng/mL EGF, F=50 ng/mL bFGF, I=50 ng/mL IGR1, C=CHIR=4 uM CHIR99021, V=VPA=1 mM Valproic Acid Sodium Salt, EPZ=123 nM EPZ011989.

FIG. 30A is a graph depicting that the EZH2 inhibitor UNC 2399 does not proliferate Lgr5 GFP+ cochlear progenitor cells in a background of growth factors. The y-axis depicts Lgr5 GFP(+) cell area and the x-axis depicts concentration of UNC 2399. Media components include 50 ng/mL EGF, 50 ng/mL bFGF, 50 ng/mL IGR1, 4 μM CHIR99021, 1 mM VPA and 0-10 μM UNC 2399.

FIG. 30B is a graph depicting that the EZH2 inhibitor UNC 2399 does not enrich for Lgr5 GFP+ cochlear progenitor cells in a background of growth factors. The y-axis depicts Lgr5 GFP(+) cell area and the x-axis depicts concentration of UNC 2399. Media components include 50 ng/mL EGF, 50 ng/mL bFGF, 50 ng/mL IGR1, 4 μM CHIR99021, 1 mM VPA and 0-10 μM UNC 2399.

FIG. 31A is a graph depicting that the EZH2 inhibitor UNC 2399 enhances Lgr5 GFP+ progenitor cell proliferation when combined with CHIR in a background of growth factors compared to CHIR alone. The y-axis depicts Lgr5 GFP(+) cell area and the x-axis depicts media conditions: E=50 ng/mL EGF, F=50 ng/mL, bFGF, I=50 ng/mL, IGR1, C=CHIR=4 μM CHIR99021, UNC=10 μM UNC 2399.

FIG. 31B is a graph depicting that the EZH2 inhibitor UNC 2399 enhances enrichment of Lgr5 GFP+ cochlear progenitor cells when combined with CHIR in a background of growth factors compared to CHIR alone. The y-axis depicts Lgr5 GFP(+) cell area percentage and the x-axis depicts media conditions: E=50 ng/mL EGF, F=50 ng/mL bFGF, I=50 ng/mL IGR1, C=CHIR=4 μM CHIR99021, UNC=10 μM UNC 2399.

FIG. 32A is a graph depicting that the EZH2 inhibitor EPZ6438 enhances Lgr5 GFP+ progenitor cell proliferation when combined with CHIR in a background of growth factors compared to CHIR alone. The y-axis depicts Lgr5 GFP(+) cell area and the x-axis depicts media conditions: E=50 ng/mL EGF, F=50 ng/mL, bFGF, I=50 ng/mL, IGR1, C=CHIR=4 μM CHIR99021, EPZ6438=370 nM EPZ6438 (tazemetostat).

FIG. 32B is a graph depicting that the EZH2 inhibitor EPZ6438 enhances enrichment of Lgr5 GFP+ cochlear progenitor cells when combined with CHIR in a background of growth factors compared to CHIR alone. The y-axis depicts Lgr5 GFP(+) cell area percentage and the x-axis depicts media conditions: E=50 ng/mL EGF, F=50 ng/mL bFGF, I=50 ng/mL IGR1, C=CHIR=4 μM CHIR99021, EPZ6438=370 nM EPZ6438 (tazemetostat).

FIG. 33A is a graph depicting that the EZH2 inhibitor CPI-169 enhances Lgr5 GFP+ progenitor cell proliferation when combined with CHIR in a background of growth factors compared to CHIR alone. The y-axis depicts Lgr5 GFP(+) cell area and the x-axis depicts media conditions: E=50 ng/mL EGF, F=50 ng/mL bFGF, I=50 ng/mL IGR1, C=CHIR=4 μM CHIR99021, CPI=1.11 μM CPI-169.

FIG. 33B is a graph depicting that the EZH2 inhibitor CPI-169 enhances enrichment of Lgr5 GFP+ cochlear progenitor cells when combined with CHIR in a background of growth factors compared to CHIR alone. The y-axis depicts Lgr5 GFP(+) cell area percentage and the x-axis depicts media conditions: E=50 ng/mL EGF, F=50 ng/mL bFGF, I=50 ng/mL IGR1, C=CHIR=4 μM CHIR99021, CPI=1.11 μM CPI-169.

FIG. 34A is a graph depicting that the DOT1L inhibitor EPZ004777 enhances Lgr5 GFP+ progenitor cell proliferation when combined with CHIR in a background of growth factors. The y-axis depicts Lgr5 GFP(+) cell count and the x-axis depicts control conditions (background growth factors plus CHIR (EFI-C) or CHIR+VPA (EFI-CV)) versus CHIR+EPZ004777 (EFI-C-EPZ). Media components include 50 ng/mL EG, 50 ng/mL bFGF, 50 ng/mL IGR1, 3 μM CHIR99021, 1 mM VPA and 15 μM EPZ004777.

FIG. 34B is a graph depicting that the DOT1L inhibitor EPZ004777 enhances enrichment of Lgr5 GFP+ cochlear progenitor cells when combined with CHIR in a background of growth factors. The y-axis depicts Lgr5 GFP(+) cell proliferation percentage and the x-axis depicts control conditions (EFI-C) or (EFI-CV) versus CHIR+EPZ004777 (EFI-C-EPZ). Media components include 50 ng/mL EG, 50 ng/mL bFGF, 50 ng/mL IGR1, 3 μM CHIR99021, 1 mM VPA and 15 μM EPZ004777.

FIG. 35A is a graph depicting that the DOT1L inhibitor SGC0946 enhances Lgr5 GFP+ progenitor cell proliferation when combined with CHIR in a background of growth factors. The y-axis depicts Lgr5 GFP(+) cell count and the x-axis depicts control conditions (EFI-C) or (EFI-CV) versus CHIR+SGC0946 (EFI-C-SGC). Media components include 50 ng/mL EG, 50 ng/mL bFGF, 50 ng/mL IGR1, 3 μM CHIR99021, 1 mM VPA and 1.7 μM SGC0946.

FIG. 35B is a graph depicting that the DOT1L inhibitor SGC0946 enhances enrichment of Lgr5 GFP+ cochlear progenitor cells when combined with CHIR in a background of growth factors. The y-axis depicts Lgr5 GFP(+) cell proliferation percentage and the x-axis depicts control conditions (EFI-C) or (EFI-CV) versus CHIR+SGC09468 (EFI-C-SGC). Media components include 50 ng/mL EG, 50 ng/mL bFGF, 50 ng/mL IGR1, 3 μM CHIR99021, 1 mM VPA and 1.7 μM SGC0946.

FIG. 36A is a graph depicting that the KDM2/7 inhibitor TC-E 5002 enhances Lgr5 GFP+ progenitor cell proliferation when combined with CHIR and VPA in a background of growth factors. The y-axis depicts Lgr5 GFP(+) cell count and the x-axis depicts control conditions (background growth factors plus CHIR (EFI-C) or CHIR and VPA (EFI-CV)) versus CHIR+VPA+TC-E 5002 (EFI-CV-TCE). Media components include 50 ng/mL EG, 50 ng/mL bFGF, 50 ng/mL IGR1, 3 μM CHIR99021, 1 mM VPA and 0.37 μM TC-E 5002.

FIG. 36B is a graph depicting that the KDM2/7 inhibitor TC-E 5002 enhances enrichment of Lgr5 GFP+ cochlear progenitor cells when combined with CHIR and VPA in a background of growth factors. The y-axis depicts Lgr5 GFP(+) cell proliferation percentage and the x-axis depicts control conditions (EFI-C) or (EFI-CV) versus CHIR+VPA+TC-E 5002 (EFI-CV-TCE). Media components include 50 ng/mL EG, 50 ng/mL bFGF, 50 ng/mL IGR1, 3 μM CHIR99021, 1 mM VPA and 0.37 μM TC-E 5002.

FIG. 37A is a graph depicting that the KDM5 inhibitor AS 8351 further enhances Lgr5 GFP+ progenitor cell proliferation when combined with CHIR and VPA in a background of growth factors. The y-axis depicts Lgr5 GFP(+) cell count and the x-axis depicts control conditions (background growth factors plus CHIR (EFI-C) CHIR and VPA (EFI-CV)) versus CHIR+VPA+AS 8351 (EFI-CV-AS). Media components include 50 ng/mL EG, 50 ng/mL bFGF, 50 ng/mL IGR1, 3 μM CHIR99021, 1 mM VPA and 2 μM AS 8351.

FIG. 37B is a graph depicting that the KDM5 inhibitor AS 8351 further enhances enrichment of Lgr5 GFP+ cochlear progenitor cells when combined with CHIR and VPA in a background of growth factors. The y-axis depicts Lgr5 GFP(+) cell proliferation percentage and the x-axis depicts control conditions (EFI-C) or (EFI-CV) versus CHIR+VPA+AS 8351 (EFI-CV-TAS). Media components include 50 ng/mL EG, 50 ng/mL bFGF, 50 ng/mL IGR1, 3 μM CHIR99021, 1 mM VPA and 2 μM AS 8351.

FIG. 38A is a graph depicting that the LSD1 inhibitor RN-1 HCl does not proliferate Lgr5 GFP+ cochlear progenitor cells in a background of growth factors compared to cells grown in a background of growth factors plus CHIR99021 (CHIR; EFI-C), CHIR and VPA (EFI-CV), or CHIR, VPA, and tranylcypromine (EFI-CVT). The y-axis depicts Lgr5 GFP(+) cell count and the x-axis depicts concentration of tranylcypromine. Media components include 50 ng/mL EGF, 50 ng/mL bFGF, 50 ng/mL IGR1, 4 μM CHIR99021, 1 mM VPA, 7 μM tranylcypromine, and 0-30 μM.

FIG. 38B is a graph depicting that the LSD1 inhibitor RN-1 HCl does not enrich Lgr5 GFP+ cochlear progenitor cells in a background of growth factors compared to cells grown in a background of growth factors plus CHIR99021 (CHIR; EFI-C), CHIR and VPA (EFI-CV), or CHIR, VPA, and tranylcypromine (EFI-CVT). The y-axis depicts Lgr5 GFP(+) cell count and the x-axis depicts concentration of tranylcypromine. Media components include 50 ng/mL EGF, 50 ng/mL bFGF, 50 ng/mL IGR1, 4 μM CHIR99021, 1 mM IPA, 7 μM tranylcypromine, and 0-30 μM.

FIG. 39A is a graph depicting that the LSD1 inhibitor RN-1 HCl further enhances Lgr5 GFP+ progenitor cell proliferation when combined with CHIR and VPA in a background of growth factors. The y-axis depicts Lgr5 GFP(+) cell count and the x-axis depicts control conditions (background growth factors plus CHIR (EFI-C), CHIR and VPA (EFI-CV), and CHIR, VPA, and tranylcypromine (EFI-CVT)) versus CHIR+VPA+RN-1 HCl (EFI-CV-RN1). Media components include 50 ng/mL EGF, 50 ng/mL bFGF, 50 ng/mL IGR1, 4 μM CHIR99021, 1 mM VPA, 7 μM tranylcypromine, and 41 nM RN-1.

FIG. 39B is a graph depicting that the LSD1 inhibitor RN-1 HCl enhances enrichment of Lgr5 GFP+ progenitor cells when combined with CHIR and VPA in a background of growth factors. The y-axis depicts Lgr5 GFP(+) cell count and the x-axis depicts control conditions (background growth factors plus CHIR (EFI-C), CHIR and VPA (EFI-CV), and CHIR, VPA, and tranylcypromine (EFI-CVT)) versus CHIR+VPA+RN-1 HCl (EFI-CV-RN1). Media components include 50 ng/mL EGF, 50 ng/mL bFGF, 50 ng/mL IGR1, 4 μM CHIR99021, 1 mM VPA, 7 μM tranylcypromine, and 41 nM RN-1.

### DETAILED DESCRIPTION

The invention is based upon the discovery that administering an epigenetic agent in combination with a Wnt agonist results in the proliferation of cochlear supporting cells or vestibular supporting cells while maintaining, in the daughter cells, the capacity to differentiate into cochlear hair cells or vestibular hair cells. Epigenetic modulation alone has no effect on the proliferation of cochlear supporting cells or vestibular supporting cells. Wnt agonists have previously been used to stimulate proliferation of supporting cells with some success. However, surprisingly, the combination of an epigenetic agent and Wnt agonist resulted in a surprising level of proliferation of cells in these contexts. Indeed, the combination of an epigenetic agent and a Wnt agonist increased proliferation of cochlear supporting cells or vestibular supporting cells relative to stimulation with either Wnt agonist or epigenetic inhibition alone. The combination of an epigenetic agent and a Wnt agonist therefore produces a larger population of expanded cochlear cells or vestibular cells compared to either Wnt agonist or epigenetic modulation alone. In other words, the combination of epigenetic modulation and a Wnt agonist is more effective at inducing self-renewal of cochlear supporting cells and vestibular supporting cells than either Wnt agonist or epigenetic modulation alone. Self-renewal of cochlear supporting cells or vestibular supporting cells, it is meant inducing the a cochlear supporting cell or vestibular supporting cell to proliferate while maintaining, in the daughter cells, the capacity to differentiate into cochlear hair cells, thus providing a therapy for treating a subject who has, or is at risk of, developing an inner ear hearing or balance disorder.

Cochlear cells have also been treated with Wnt agonists in combination with valproic acid (VPA) (McLean et al. 2017). The inventors have found that replacement of valproic acid with another epigenetic agent is advantageous because such epigenetic agents can be effective at lower concentrations than VPA, potentially simplifying delivery of the epigenetic agent to the inner ear, as well as minimizing the risk of side effects. Moreover, the combination of an alternative epigenetic agent and Wnt agonist can produce a greater population of expanded cells compared to the combination of a Wnt agonist and VPA.

The methods described herein can increase the proliferation of cochlear supporting cells or vestibular supporting cells. Typically, the cochlear supporting cell or vestibular supporting cell in which proliferation is stimulated expresses Lgr5 (Leucine-rich repeat-containing G-protein coupled receptor 5). However, the methods described herein may also stimulate proliferation of supporting cells with little or no Lgr5 expression.

The methods described can herein produce an expanded population of cochlea or vestibular cells. In some embodiments, the expanded cells are enriched for Lgr5 expression (i.e. a greater percentage of the expanded cell population express Lgr5 compared to the starting cell population).

Lgr5 is a member of GPCR class A receptor proteins that is expressed across a diverse range of tissues such as in the muscle, placenta, spinal cord and brain, and particularly as a biomarker of adult stem cells in certain tissues. Lgr5+ stem cells are the precursors for sensory hair cells that are present in cochlea and vestibular organs of the inner ear. Increasing the population of Lgr5+ cochlear or vestibular cells is therefore beneficial because it increases the population of precursor cells which may differentiate into sensory hair cells.

The present invention provides compositions and methods for inducing the self-renewal of a cochlear supporting cells and vestibular supporting cells by by decreasing LSD1, EZH2, DOT1L, and/or KDM expression or activity in combination with a Wnt agonist.

Thus, in various aspects the invention provides compositions and methods for increasing proliferation of a cochlear supporting cell or vestibular supporting cell; producing an expanded population of cochlear or vestibular cells and treating an inner ear hearing or balance disorder in a subject by contacting a cochlear supporting cell or vestibular supporting cell, or administering to a subject, an LSD1 inhibitor EZH2, DOT1L, and/or KDM and a Wnt Agonist.

In another aspect of the invention, the cochlear supporting cell or vestibular supporting cell is further contacted with, or a subject is further administered with, a second epigenetic agent. In some embodiments, the second epigenetic agent is an HDAC inhibitor, for example valproic acid (VPA). The addition of a second epigenetic agent to the first epigenetic agent and Wnt agonist is advantageous because proliferation of the supporting cell population can be increased compared to the combination of either an epigenetic agent and Wnt agonist or Wnt agonist and valproic acid. In some embodiments, the expanded population of cells that can be produced following treatment with an epigenetic agent, a Wnt agonist and a second epigenetic agent is larger than the expanded population of cells that can be produced compared to the combination of either LSD1 inhibitor and Wnt agonist or Wnt agonist and valproic acid. The Lgr5+ cell population can be more enriched when a second epigenetic agent is used compared to the combination of a single epigenetic agent and a Wnt agonist, or the combination of a Wnt agonist and an HDAC inhibitor,

Epigenetic Agents

Epigenetic agents are agents that can modulate activity of epigenetic modifiers, mediators and modulators. Epigenetic modifiers are genes whose products modify the epigenome directly through DNA methylation, the post-translational modification of chromatin or the alteration of the structure of chromatin. Epigenetic mediators, are often the target of epigenetic modification, although they are rarely mutated themselves. The epigenetic mediators largely overlap with the genes involved in stem cell reprogramming and their role in cancer followed directly from the discovery of their reprogramming role. Epigenetic mediators are those genes whose products are the targets of the epigenetic modifiers. Epigenetic modulators are the genes lying upstream of the modifiers and mediators in signalling and metabolic pathways

In some embodiments, an agent having activity as an epigenetic agent is an HDAC inhibitor, an EZH2 inhibitor, an LSD1 inhibitor, a DOT1L inhibitor, or KDM inhibitor.

### LSD1 Inhibitors

LSD1 mediated H3K4 demethylation can result in a repressive chromatin environment that silences gene expression. LSD1 has been shown to play a role in development in various contexts. LSD1 can interact with pluripotency factors in human embryonic stem cells and is important for decommissioning enhancers in stem cell differentiation. Beyond embryonic settings, LSD1 is also critical for hematopoietic differentiation. LSD1 is overexpressed in multiple cancer types and recent studies suggest inhibition of LSD1 reactivates the all-trans retinoic acid receptor pathway in acute myeloid leukemia (AML). These studies implicate LSD1 as a key regulator of the epigenome that modulates gene expression through post-translational modification of histones and through its presence in transcriptional complexes.

Thus, a “LSD1 inhibitor” refers to an agent capable of the decreasing the expression or enzymatic activity of LSD1. For example a LSD1 inhibitor results in a decrease in H3K4 demethylation of a target gene in a cell, for instance, in a cochlear cell or a vestibular. cell

In certain embodiments, a LSD1 inhibitor decreases the expression or enzymatic activity of LSD1 by at least 5, 10, 20, 30, 40, 50, 60, 70, 80, 90, or 100% relative to a control, for example relative to a baseline level of activity.

In certain embodiments, a LSD1 inhibitor decreases H3K4 demethylation by at least 5, 10, 20, 30, 40, 50, 60, 70, 80, 90, or 100% relative to a control, for example relative to a baseline level of activity.

In some instances, a LSD1 inhibitor decreases H3K4 demethylation by at least about 1.1, 1.2, 1.3, 1.4, 1.5, 1.6, 1.7, 1.8, 1.9, 2, 3, 4, 5, 6, 7, 8, 9, 10, 15, 20, 30, 40, 50, 60, 70, 80, 90, 100, 200, 500, 1000-fold or more relative to a control, for example relative to a baseline level of activity.

In some instances, a LSD1 inhibitor modulates (i.e. increases or decreases) expression or activity of a target gene by at least 5, 10, 20, 30, 40, 50, 60, 70, 80, 90, or 100% relative to a control, for example relative to a baseline level of activity.

In some instances, a LSD1 inhibitor modulates (i.e. increases or decreases) expression or enzymatic activity of LSD1 by at least about 1.1, 1.2, 1.3, 1.4, 1.5, 1.6, 1.7, 1.8, 1.9, 2, 3, 4, 5, 6, 7, 8, 9, 10, 15, 20, 30, 40, 50, 60, 70, 80, 90, 100, 200, 500, 1000-fold or more relative to a control, for example relative to a baseline level of activity.

In some instances a LSD1 inhibitor is reversible. In other instances the LSD1 inhibitor is irreversible.

Exemplary agents having activity as a LSD1 inhibitors are provided in Table 1 below, including pharmaceutically-acceptable salts thereof.

TABLE 1

| | | pKi | | Reversible | | | | Select | |
|---|---|---|---|---|---|---|---|---|---|
| | | or | | or | Chemo- | Select | | MAOs | |
| Agent | CAS | IC50 | | Irreversible | type | KDM1b | | A and B | |
| GSK-2879552 | 1401966-69-5 | 1.7 | μM | Irreversible | Cyproylamine | | | 20 | μM |
| | | (0.11 | μM) | | | | | | |
| GSK-LSD1 | 1431368-48-7 | 16 | nM | Irreversible | Cyproylamine | >1000X | | >1000X | |
| Phenelzine | 51-71-8 | 5.6 | μM | Irreversible | Hydrazine | | | MAO | |
| sulfate | | | | | | | | inhib | |
| | | | | | | | | 900 nM | |
| | | | | | | | | in Cell | |
| TCP | 155-09-9 | 11-477 | μM | Ineversible | Cyproylamine | 186 | μM | 1 | μM |
| (Tranylcypromine) | | | | | | | | | |
| ORY-1001 | 1431326-61-2 | <20 | nM | Irreversible | Cyproylamine | >100 | μM | >100 | μM |
| (RG6016, | | | | | | | | >100 | μM |
| RO7051790, | | | | | | | | | |
| Iadademstat) | | | | | | | | | |
| RN-1 | 1781835-13-9 | 70 | nM | Irreversible | Cyproylamine | | | 0.51 | μM |
| | | | | | | | | 2.78 | μM |
| CC-90011 | 2179319-65-2 | | | Reversible | Likely | | | | |
| | | | | | pyrimidinyl | | | | |
| GCG-11047 | 308145-19-9 | | | Reversible | Polyamine | | | | |
| (PG-11047) | | | | | | | | | |
| IMG-7289 | 2229826-41-7 | | | Ineversible | Likely | | | | |
| | | | | | Cyproylamine | | | | |
| INCB059872 | 1802909-49-4 | | | Ineversible | Likely | | | | |
| | | | | | Cyproylamine | | | | |
| ORY-2001 | 1357362-02-7 | | | Ineversible | Cyproylamine | | | | |
| (Vafidemstat) | | | | | | | | | |
| Osimertinib | 1421373-65-0 | 3.98 | μM | Reversible | Pyrimidinyl | | | | |
| (AZD9291) | | | | | | | | | |
| SP-2577 | 1423715-37-0 | | | Ineversible | Hydrazone | | | | |
| (Seclidemstat) | 1821307-10-1 | | | | | | | | |
| TCP Trans | 3721-28-6 | 284 | μM | Ineversible | Cyproylamine | 137 | μM | B: 4.4 | μM |
| Chiral | | | | | | | | | |
| TCP Trans | 3721-26-4 | 168 | μM | Ineversible | Cyproylamine | 127 | μM | B: 89 | μM |
| Chiral | | | | | | | | | |
| TCP Cis | 13531-35-6 | | | Ineversible | Cyproylamine | | | 11 | μM |
| | | | | | | | | 19 | μM |
| TCP Cis Chiral | 69684-88-4 | | | Ineversible | Cyproylamine | | | | |
| TCP Cis Chiral | 69684-89-5 | | | Ineversible | Cyproylamine | | | | |
| Compound 1 | 1221595-26-1 | 10 | nM | Ineversible | Cyproylamine | | | | |
| Compound 45 | 1667721-01-8 | 9 | nM | Ineversible | Cyproylamine | | | 15 | μM |
| | | | | | | | | >40 | μM |
| RN-7 | 1352345-82-4 | 31 | nM | Ineversible | Cyproylamine | | | | |
| Compound 5A | 1613476-09-7 | 12 | nM | Ineversible | Cyproylamine | | | | |
| Compound 2 | 1235863-51-0 | 67 | nM | Ineversible | Cyproylamine | | | >37 | μM |
| Compound 43 | 1784703-61-2 | 610 | nM | Ineversible | Cyproylamine | | | | |
| Compound 12f | 1802319-25-0 | 86 | nM | Ineversible | Cyproylamine | | | 460 | nM, |
| | | | | | | | | >70 | μM |
| T-3775440 | 1422620-34-5 | 2.1 | nM | Ineversible | Cyproylamine | | | 110 | μM, |
| | | | | | | | | 17 | μM |
| OG-L002 | 1357299-45-6 | 20 | nM | Ineversible | Cyproylamine | | | | |
| S2101 | 1239262-36-2 | 990 | nM | Ineversible | Cyproylamine | | | | |
| NCL-1 | 1196119-03-5 | 1.6 | μM | Ineversible | Cyproylamine | | | | |
| Compound 9A | 2095849-74-2 | 1.2 | μM | Ineversible | Cyproylamine | | | | |
| Compound 19l | 2173543-81-0 | 0.97 | μM | Ineversible | Cyproylamine | | | | |
| NCD-25 | 1456972-46-5 | 480 | nM | Ineversible | Cyproylamine | | | | |
| NCD-38 | 2078047-42-2 | 590 | nM | Ineversible | Cyproylamine | | | | |
| Compound 14A | 2247939-53-1 | 2.2 | nM | Ineversible | Cyproylamine | | | | |
| Compound 15A | 2247939-55-3 | 70 | nM | Ineversible | Cyproylamine | | | | |
| Compound 15B | 2247939-56-4 | 11 | nM | Ineversible | Cyproylamine | | | | |
| Compound 4 | 2226461-60-3 | 43 | nM | Ineversible | Cyproylamine | | | | |
| Pargyline | 555-57-7 | 1000 | μM | Ineversible | Amino-propyne | | | | |
| Peptide | 945548-35-6 | | | Ineversible | Amino-propyne | | | | |
| Bizine | 1591932-50-1 | 59 | nM | Ineversible | Hydrazine | | | | |
| Compound 5a | 1990536-90-7 | 1.4 | nM | Reversible | Hydrazone | | | | |
| Compound 5n | 1990537-03-5 | 1.7 | nM | Reversible | Hydrazone | | | | |
| SP-2509 | 1423715-09-6 | 13 | nM | Reversible | Hydrazone | | | >300 | μM |
| (HCI-2509) | | | | | | | | | |
| LSD1IN-32 | 2137044-49-4 | 83 | nM | Reversible | Amide | | | | |
| LSD1IN-11p | 2101951-67-9 | 20-80 | nM | Reversible | Pyrazole | | | | |
| Resveratrol | 501-36-0 | 15 | μM | Reversible | Resveratrol | | | | |
| Hydroxylamine | 2035912-55-9 | 121 | nM | Reversible | Resveratrol | | | | |
| Compound 8c | 2170023-28-4 | 283 | nM | Reversible | Resveratrol | | | | |
| CBB-1007 | 1379573-92-8 | 2.1 | μM | Reversible | Polyamine | | | | |
| Namoline | 342795-11-3 | 51 | μM | Reversible | Benzopyran-4-one | | | | |
| GSK-354 | 1841508-96-0 | 29-80 | nM | Reversible | Diphpyridine | | | A > 50 μM | |
| | | | | | | | | B = 19 μM | |
| GSK-690 | 2101305-84-2 | 37 | nM | Reversible | Diphpyridine | | | | |
| E11 | 1239589-91-3 | 243 | nM | Reversible | 2,4- | | | | |
| | | | | | Quinazolinediamine | | | | |
| MC2694 | 1435055-66-5 | 1 | μM | Reversible | 2,4- | | | | |
| | | | | | Quinazolinediamine | | | | |
| Alpha- | 11/1/6147 | 2.8 | μM | Reversible | mangostin | | | | |
| mangostin | | | | | | | | | |
| Compound 12 A | 1923750-07-5 | 0.41 | μM | Reversible | Barbituate | | | | |
| Compound 4 | 126118-57-8 | 6.4 | μM | Reversible | Purine-2,6-dione | | | | |
| Compound 10d | 2226997-31-3 | 4 | μM | Reversible | Carboxamide | | | | |
| Compound 90 | 1884266-15-2 | 162 | nM | Reversible | Carboxamide | | | | |
| Compound 46 | 1884266-36-7 | 8 | nM | Reversible | Carboxamide | | | | |
| Compound 49 | 1884266-49-2 | 7 | nM | Reversible | Carboxamide | | | | |
| Compound 50 | 1884266-48-1 | 8 | nM | Reversible | Carboxamide | | | | |
| Polymyxin B | 1404-26-8 | 157 | nM | Reversible | Polymyxin B | | | | |
| Polymyxin E | 1066-17-7 | 193 | nM | Reversible | Polymyxin E | | | | |
| Baicalin | 21967-41-9 | 3.0 | μM | Reversible | Baicalin | | | | |
| Compound 16Q | 1612870-90-2 | 9.5 | uM | Reversible | Benzenesulfonamide | | | >500 | μM |
| LSD1 inhibitor | 1853269-07-4 | 7 | nM | Reversible | Imidazole | | | | |
| 24 | | | | | | | | | |
| geranylgeranoic | 35750-48-2 | 120 | μM | Reversible | Geranyl | | | | |
| acid | | | | | | | | | |
| Geranylgeraniol | 24034-73-9 | 80 | μM | Reversible | Geranyl | | | | |
| Thiocarbamate | 1430852-56-4 | 390 | nM | Reversible | Thiocarbamate | | | >1250 | μM |
| Thiourea | 1637373-61-5 | 650 | nM | Reversible | Thiourea | | | >1250 | μM |
| Thiourea | 2035417-23-1 | 154 | nM | Reversible | Thiourea | | | | |
| Thienopyrrole | 1206028-57-0 | 2.9 | μM | Reversible | Thienopyrrole | >100 | μM | >100 | uM, |
| | | | | | | | | 57 | μM |
| Thienopyrrole | 1884266-15-2 | 162 | nM | Reversible | Thienopyrrole | | | | |
| Thienopyrrole | 1884266-48-1 | 7.8 | nM | Reversible | Thienopyrrole | 13 | μM | 41 | uM, |
| | | | | | | | | 100 | μM |
| 4SC-202 | 910462-43-0 | 1-10 | μM | Reversible | o-aminoph | | | | |
| ORY-3001 | 2179325-30-3 | | | | | | | | |
| JL1037 | | | | | | | | | |
| FLI-06 | 313967-18-9 | 92 | nM | Inhibits | Dihydropyridine | | | | |
| | | | | expression | | | | | |
| | | | | of ESD1 | | | | | |
| Rhodium | | 40 | nM | | Rhodium | | | | |
| Complex 1 | | | | | | | | | |

| | | | | | Formul. | Human | |
|---|---|---|---|---|---|---|---|
| | Literat. | | Lgr5+ | Perilymph | Conc. | Plasma | Human |
| Agent | Cell | | Assay | Conc. | Intraymp. | Conc. | Dosage |
| GSK-2879552 | 2-240 | nM | 40 nM- | 40 nM- | 40 μM to | 1-100 nM | 1 or 2 mg |
| | | | 30 μM | 30 μM | 30 mM | | QD PO |
| GSK-LSD1 | | | 4 nM- | 4 nM- | 4 μM to | 1-100 nM | 10-100 mg |
| | | | 50 μM | 50 μM | 50 mM | | PO |
| Phenelzine | 900 | | | 0.1-10 μM | 0.1-10 | Cmax | 15-90 mg/day |
| sulfate | nM in | | | | mM | 10 to | PO |
| | Cell | | | | | 60 ng/mL | |
| | | | | | | (73-440 nM) | |
| TCP | | | 0.1-20 μM | 0.1-20 μM | 0.1-20 | Cmax | 15-150\mg/day |
| (Tranylcypromine) | | | | | mM | 30-200 ng/ml | PO |
| | | | | | | (225-1500 nM) | |
| ORY-1001 | 0.5-3 | nM | 41 nM | 4 nM- | 4 uM to | 1-100 nM | 1-100 mg |
| (RG6016, | | | | 50 μM | 50 mM | | PO |
| RO7051790, | | | | | | | |
| Iadademstat) | | | | | | | |
| RN-1 | | | 41 nM | 4 nM- | 4 uM to | 1-100 nM | 10-100 mg |
| | | | | 50 μM | 50 mM | | PO |
| CC-90011 | | | | | | | |
| GCG-11047 | | | | | | | |
| (PG-11047) | | | | | | | |
| IMG-7289 | | | | | | Cmax | 80 mg |
| | | | | | | 63 ng/ml | QD PO |
| INCB059872 | | | | | | | |
| ORY-2001 | | | | | | | |
| (Vafidemstat) | | | | | | | |
| Osimertinib | 43 | nM | | | | | 10-80 mg |
| (AZD9291) | | | | | | | |
| SP-2577 | | | | | | | |
| (Seclidemstat) | | | | | | | |
| TCP Trans | | | | | | | |
| Chiral | | | | | | | |
| TCP Trans | | | | | | | |
| Chiral | | | | | | | |
| TCP Cis | | | | | | | |
| TCP Cis Chiral | | | | | | | |
| TCP Cis Chiral | | | | | | | |
| Compound 1 | | | | | | | |
| Compound 45 | | | | | | | |
| RN-7 | | | | | | | |
| Compound 5A | | | | | | | |
| Compound 2 | | | | | | | |
| Compound 43 | | | | | | | |
| Compound 12f | | | | | | | |
| T-3775440 | | | | | | | |
| OG-L002 | | | | | | | |
| S2101 | | | | | | | |
| NCL-1 | | | | | | | |
| Compound 9A | | | | | | | |
| Compound 19l | | | | | | | |
| NCD-25 | | | | | | | |
| NCD-38 | | | | | | | |
| Compound 14A | | | | | | | |
| Compound 15A | | | | | | | |
| Compound 15B | | | | | | | |
| Compound 4 | 3.8 | μM | | | | | |
| Pargyline | | | | | | | |
| Peptide | | | | | | | |
| Bizine | | | | | | | |
| Compound 5a | | | | | | | |
| Compound 5n | | | | | | | |
| SP-2509 | 350-650 | nM | | | | | |
| (HCI-2509) | | | | | | | |
| LSD1IN-32 | 670 | nM | | | | | |
| LSD1IN-11p | 0.52 | μM | | | | | |
| Resveratrol | | | | | | | |
| Hydroxylamine | | | | | | | |
| Compound 8c | 5 to 9 | μM | | | | | |
| CBB-1007 | IC50 ≤ 5 μM | | | | | | |
| Namoline | | | | | | | |
| GSK-354 | 1.3 | μM | | | | | |
| GSK-690 | | | | | | | |
| E11 | | | | | | | |
| MC2694 | | | | | | | |
| Alpha- | | | | | | | |
| mangostin | | | | | | | |
| Compound 12 A | | | | | | | |
| Compound 4 | | | | | | | |
| Compound 10d | | | | | | | |
| Compound 90 | | | | | | | |
| Compound 46 | 1-4 | μM | | | | | |
| Compound 49 | 1-4 | μM | | | | | |
| Compound 50 | 1-4 | μM | | | | | |
| Polymyxin B | | | | | | | |
| Polymyxin E | | | | | | | |
| Baicalin | | | | | | | |
| Compound 16Q | | | | | | | |
| LSD1 inhibitor | | | | | | | |
| 24 | | | | | | | |
| geranylgeranoic | | | | | | | |
| acid | | | | | | | |
| Geranylgeraniol | | | | | | | |
| Thiocarbamate | | | | | | | |
| Thiourea | | | | | | | |
| Thiourea | | | | | | | |
| Thienopyrrole | | | | | | | |
| Thienopyrrole | | | | | | | |
| Thienopyrrole | | | | | | | |
| 4SC-202 | | | | | | | 25-400 mg/Day |
| ORY-3001 | | | | | | | |
| JL1037 | | | | | | | |
| FLI-06 | | | | | | | |
| Rhodium | | | | | | | |
| Complex 1 | | | | | | | |

In some embodiments, an agent of having activity as a LSD1 inhibitor is GSK-2879552, GSK-LSD1, Osimertinib (AZD9291), Phenelzine sulfate, Tranylcypromine (TCP), RN-1, ORY-1001, Seclidemstat (SP-2577), Vafidemstat (ORY-2001), CC-90011, IMG-7289 or, INCB059872. In some embodiments, the LSD1 inhibitor is GSK-2879552, GSK-LSD1, Tranylcypromine, Phenelzine sulfate, RN-1, or ORY-1001.

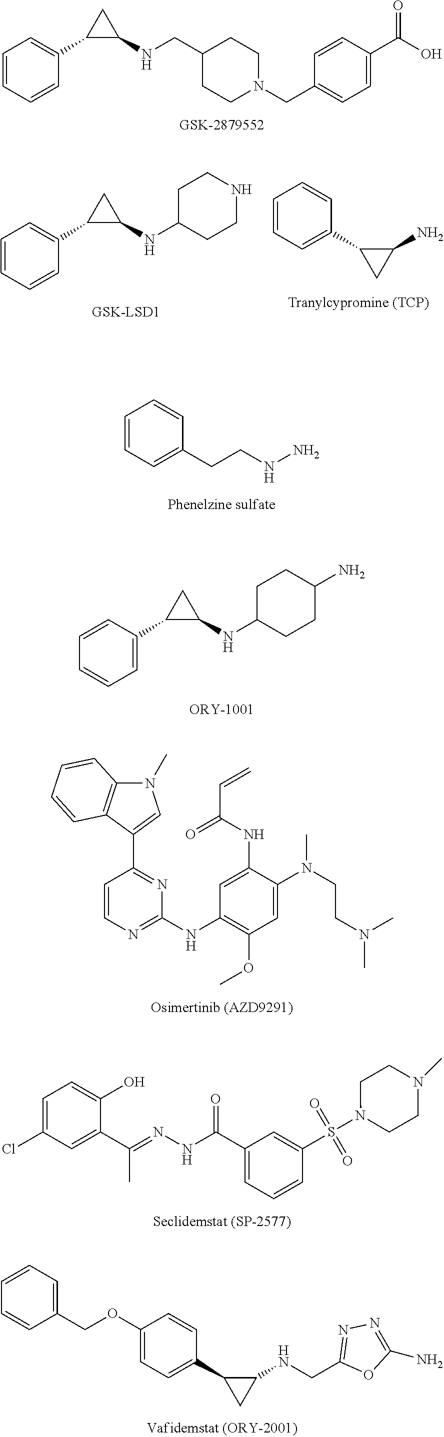

In some embodiments, the LSD1 inhibitor is GSK-2879552 or Tranylcypromine (TCP).

EZH2 Inhibitors

Enhancer of zeste homolog 2 (EZH2) is a histone-lysine N-methyltransferase enzyme encoded by EZH2 gene, that participates in histone methylation and, ultimately, transcriptional repression. EZH2 catalyzes the addition of methyl groups to histone H3 at lysine 27, by using the cofactor S-adenosyl-L-methionine. Methylation activity of EZH2 facilitates heterochromatin formation thereby silences gene function. Remodeling of chromosomal heterochromatin by EZH2 is also required during cell mitosis.

EZH2 is the functional enzymatic component of the Polycomb Repressive Complex 2 (PRC2), which is responsible for healthy embryonic development through the epigenetic maintenance of genes responsible for regulating development and differentiation EZH2 is responsible for the methylation activity of PRC2, and the complex also contains proteins required for optimal function (EED, SUZ12, JARID2, AEBP2, RbAp46/48, and PCL).

EZH2 inhibitors are chemical compounds that inhibit histone-lysine N-methyltransferase enzyme encoded by EZH2 gene

Thus, “EZH2 inhibitor” refers to an agent capable of the decreasing the expression or enzymatic activity of EZH2. For example, an EZH2 inhibitor results in a decrease in histone methylation of a target gene in a cell.

In certain embodiments, the EZH2 inhibitor decreases the expression or enzymatic activity of EZH2 by at least 5, 10, 20, 30, 40, 50, 60, 70, 80, 90, or 100% relative to a control, for example relative to a baseline level of activity.

In certain embodiments, the EZH2 inhibitor decreases histone methylation of a target gene by at least 5, 10, 20, 30, 40, 50, 60, 70, 80, 90, or 100% relative to a control, for example relative to a baseline level of activity.

In some embodiments, the EZH2 inhibitor increases expression or activity of a target gene by at least 5, 10, 20, 30, 40, 50, 60, 70, 80, 90, or 100% relative to a control, for example relative to a baseline level of activity.

In some embodiments, the EZH2 inhibitor decreases expression or enzymatic activity of EZH2 by at least about 1.1, 1.2, 1.3, 1.4, 1.5, 1.6, 1.7, 1.8, 1.9, 2, 3, 4, 5, 6, 7, 8, 9, 10, 15, 20, 30, 40, 50, 60, 70, 80, 90, 100, 200, 500, 1000-fold or more relative to a control, for example relative to a baseline level of activity.

In some embodiments, the EZH2 inhibitor decreases histone methylation of a target gene by at least about 1.1, 1.2, 1.3, 1.4, 1.5, 1.6, 1.7, 1.8, 1.9, 2, 3, 4, 5, 6, 7, 8, 9, 10, 15, 20, 30, 40, 50, 60, 70, 80, 90, 100, 200, 500, 1000-fold or more relative to a control, for example relative to a baseline level of activity.

In some embodiments, the EZH2 inhibitor increases expression or activity of a target gene by at least about 1.1, 1.2, 1.3, 1.4, 1.5, 1.6, 1.7, 1.8, 1.9, 2, 3, 4, 5, 6, 7, 8, 9, 10, 15, 20, 30, 40, 50, 60, 70, 80, 90, 100, 200, 500, 1000-fold or more relative to a control, for example relative to a baseline level of activity.

Exemplary EZH2 inhibitors are provided in Table 7

TABLE 7

| | | pKi | | | | Select | | Lit | |
|---|---|---|---|---|---|---|---|---|---|
| | | or | | Enzymatic/ | | vs | | Cell | |
| Agent | CAS | IC50 | | Non-enzymatic | Chemo-type | EZH-1 | | Poten | |
| PF-06821497 | 1844849-10-0 | <1 | nM | enzymatic | 2-Pyridone | 70 | nM | 4-6 | nM |
| PF-06726304 | 1616287-82-1 | 1.1 | nM | enzymatic | 2-Pyridone | | | 10-25 | nM |
| CPI-1205 | 1621862-70-1 | 2.2 | nM | enzymatic | 2-Pyridone | 24x | | 32 | nM |
| valemetostat | 1809336-39-7 | 2.5 | nM | enzymatic | 2-Pyridone | 8.4 | nM | 25-250 | nM |
| (DS-3201b, | | | | | | | | | |
| (R)-OR-S2) | | | | | | | | | |
| tazemetostat | 1403254-99-8 | 2.5 | nM | enzymatic | 2-Pyridone | 35x | | | |
| (EPZ-6438) | | | | | | | | | |
| E11 | 1418308-27-6 | 13 | nM | enzymatic | 2-Pyridone | 90x | | | |
| CPI-169 | 1450655-76-1; | 0.24 | nM | enzymatic | 2-Pyridone | 6 | nM | | |
| | 1802175-07-0 | | | | | | | | |
| CPI-360 | 1802175-06-9 | 0.5 | nM | enzymatic | 2-Pyridone | ~50 | nM | | |
| EPZ-011989 | 1598383-40-4 | <3 | nM | enzymatic | 2-Pyridone | | | 94 | nM |
| UNC 2399 | 1433200-53-3 | | | enzymatic | 2-Pyridone | | | | |
| (R)-OR-S1 | 1809336-19-3 | 10 | nM | enzymatic | 2-Pyridone | 7.4 | nM | | |
| A-395 | 2089148-72-9 | 0.3 | nM | EED Inhibit | Amino | | | 90 | nM |
| | | | | | pyrrolidines | | | | |
| Astemizole | 68844-77-9 | 94 | μM | EED Inhibit | Benzimidazole | | | | |
| Compound 19 | 2079895-22-8 | 1.3 | μM | EED Inhibit | Imidazole | | | 1.9 | μM |
| Compound 22 | 1802175-07-0 | 2 | nM | enzymatic | 2-Pyridone | | | | |
| Compound 24 | 1659298-29-9 | 40 | nM | enzymatic | 2-Pyridone | | | | |
| Compound 34 | 2055347-72-1 | 29 | nM | enzymatic | 2-Pyridone | >100x | | | |
| Compound 41 | 2055347-94-7 | 11 | nM | enzymatic | 2-Pyridone | >100x | | | |
| CPI-0209 | | | | | | | | | |
| CPI-360 | 1802175-06-9 | 0.5 | nM | enzymatic | 2-Pyridone | ~50 | nM | | |
| EBI-2511 | 2098546-05-3 | 4 | nM | enzymatic | 2-Pyridone | | | | |
| EED162 | 1010897-73-0 | 30 | nM | EED Inhibit | Triazo | | | 80 | nM |
| EED226 | 2083627-02-3 | | | EED Inhibit | Triazo | | | | |
| EPZ-005687 | 1396772-26-1 | 24 | nM | enzymatic | 2-Pyridone | 50X | | | |
| EPZ-011989 | 1598383-40-4 | <3 | nM | enzymatic | 2-Pyridone | | | 94 | nM |
| GSK126 | 1346574-57-9 | <3 | nM | enzymatic | 2-Pyridone | 150x | | | |
| GSK343 | 1346704-33-3 | 1.2 | nM | enzymatic | 2-Pyridone | 60x | | 174 | nM |
| GSK503 | 1346572-63-1 | <10 | nM | enzymatic | 2-Pyridone | | | | |
| GSK926 | 1346704-13-9 | 7.9 | nM | enzymatic | 2-Pyridone | | | 324 | nM |
| MAK683 | 1951408-58-4 | | | EED inhibitor | Triazo | | | | |
| (EED162) | (likely patent) | | | | | | | | |
| SHR2554 | 2098545-98-1 | | | enzymatic | 2-Pyridone | | | | |
| SKLB1049 | 1826865-42-2 | 7.2 | nM | enzymatic | 2-Pyridone | | | 12 | μM |
| ZLD1039 | 1826865-46-6 | <15 | nM | enzymatic | 2-Pyridone | | | | |
| ZLD1122 | 1826865-51-3 | <15 | nM | enzymatic | 2-Pyridone | | | | |
| | 1404094-15-0 | 74 | nM | enzymatic | 2-Pyridone | | | 2510 | nM |
| | 1404094-16-1 | 14 | nM | enzymatic | 2-Pyridone | | | 1995 | nM |
| DZNep | 102052-95-9 | | | SAH-hydrolase | SAH derived | | | 1 | μM |
| | | | | inhibitor | | | | | |
| Cmpd 44 | 1378002-93-7 | 32 | nM | SAM Comp | Benzamide | | | 9 | μM |
| Compound 27 | 1676100-59-6 | 270 | nM | SAH-hydrolase | SAH derived | | | | |
| | | | | inhibitor | | | | | |
| Sinefungin | 58944-73-3 | 20 | nM | SAH-hydrolase | SAH derived | | | 33 | nM |
| | | | | inhibitor | | | | | |
| Tanshindiol B | 97465-70-8 | 520 | nM | enzymatic | Tanshindiols | | | | |
| Tanshindiol C | 97465-71-9 | 550 | nM | enzymatic | Tanshindiols | | | | |
| UN C1999 | 1431612-23-5 | 10 | nM | enzymatic | 2-Pyridone | 10x | | 124 | nM |
| (−)- | 989-51-5 | | | enzymatic | a, b-unsat | | | | |
| Epigallocatechin- | | | | | | | | | |
| 3-gallate (EGCG) | | | | | | | | | |
| Curcumin | 458-37-7 | | | enzymatic | a, b-unsat | | | | |
| MC1945 | 169903-68-8 | | | enzymatic | a, b-unsat | | | | |
| MC1947 | 949090-12-4 | | | Non-enzymatic | | | | | |
| MC1948 | 949090-20-4 | | | Non-enzymatic | | | | | |
| SAH-EZH2 | | | | Non-enzymatic | reactive | | | | |
| Sulforaphane | 4478-93-7 | | | EED Inhibit | Stapled | | | | |
| | | | | | Peptide | | | | |

| | | | | Formul. | Human | | |
|---|---|---|---|---|---|---|---|
| | Lgr5+ | Perilymph | | Conc. | In Vivo | | Human |
| Agent | Assay | Conc | | Intraymp | Conc | | Dosage |
| PF-06821497 | | 10-1000 | nM | 10-1000 | 5-50 | nM | 75 mg to |
| | | | | μM | | | 625 mg |
| | | | | | | | BID PO |
| PF-06726304 | 100 nM- | 100 nM- | | 100 μM- | 100 nM- | | 100 to |
| | 3 μM | 3 μM | | 3 mM | 30 μM | | 1000/day |
| | | | | | | | mg PO |
| CPI-1205 | 100 nM- | 0.10-30 | μM | 100- | 25-250 | μM | 800 mg |
| | 30 μM | | | 30,000 μM | | | BID and |
| | | | | | | | subsequently |
| | | | | | | | TID - PO |
| valemetostat | | 10-1000 | nM | 10- | 25-250 | nM | PO |
| (DS-3201b, | | | | 1000 μM | | | dose |
| (R)-OR-S2) | | | | | | | starting |
| | | | | | | | of 100 mg |
| | | | | | | | QD with |
| | | | | | | | dose escal |
| | | | | | | | dep on tox |
| tazemetostat | 0.1-10 μM | 0.1-10 | μM | 0.1-10 mM | 100-800 ng/ml | | PO 100 |
| (EPZ-6438) | | | | | (200-1600 nM) | | BID to |
| | | | | | | | 800 mg |
| | | | | | | | BID |
| E11 | 0.1-10 μM | 0.1-10 | μM | 0.1-10 mM | 1-10 | μM | (100 to |
| | | | | | | | 1000/day |
| | | | | | | | mg PO) |
| CPI-169 | 0.1-10 μM | 0.1-10 | μM | 0.1-10 mM | 1-10 | μM | 100 to |
| | | | | | | | 1000/day |
| | | | | | | | mg PO |
| CPI-360 | 0.1-10 μM | 0.1-10 | μM | 0.1-10 mM | 1-10 | μM | 100 to |
| | | | | | | | 1000/day |
| | | | | | | | mg PO |
| EPZ-011989 | 100 nM- | 0.10-30 | μM | 100- | 0.10-30 | μM | 100 to |
| | 30 μM | | | 30,000 μM | | | 1000/day |
| | | | | | | | mg PO |
| UNC 2399 | 0.1-10 μM | 0.1-10 | μM | 0.1-10 mM | 0.1-100 | μM | 100 to |
| | | | | | | | 1000/day |
| | | | | | | | mg PO |
| (R)-OR-S1 | | | | | | | IV 50 mg - |
| | | | | | | | poor oral |
| | | | | | | | bio. |
| A-395 | | | | | | | 50 mg and |
| | | | | | | | 200 mg PO |
| Astemizole | | | | | | | |
| Compound 19 | | | | | | | |
| Compound 22 | | | | | | | |
| Compound 24 | | | | | | | |
| Compound 34 | | | | | | | |
| Compound 41 | | | | | | | |
| CPI-0209 | | | | | | | |
| CPI-360 | | | | | | | |
| EBI-2511 | | | | | | | |
| EED162 | | | | | | | |
| EED226 | Active | | | | | | |
| EPZ-005687 | | | | | | | |
| EPZ-011989 | | | | | | | |
| GSK126 | | | | | | | |
| GSK343 | | | | | | | |
| GSK503 | | | | | | | |
| GSK926 | | | | | | | |
| MAK683 | | | | | | | |
| (EED162) | | | | | | | |
| SHR2554 | | | | | | | |
| SKLB1049 | | | | | | | |
| ZLD1039 | | | | | | | |
| ZLD1122 | | | | | | | |
| DZNep | Active | | | | | | |
| Cmpd 44 | | | | | | | |
| Compound 27 | | | | | | | |
| Sinefungin | | | | | | | |
| Tanshindiol B | | | | | | | |
| Tanshindiol C | | | | | | | |
| UN C1999 | Active | | | | | | |
| (−)- | | | | | | | |
| Epigallocatechin- | | | | | | | |
| 3-gallet (EGCG) | | | | | | | |
| Curcumin | | | | | | | |
| MC1945 | | | | | | | |
| MC1947 | | | | | | | |
| MC1948 | | | | | | | |
| SAH-EZH2 | | | | | | | |
| Sulforaphane | | | | | | | |

In some embodiments the EZH2 inhibitor is PF-06821497, PF-06726304, CPI-1205, valemetostat, tazemetostat, E11, CPI-360, EPZ011989, or UNC 2399.

### DOT1L Inhibitors

DOT1-like (Disruptor of telomeric silencing 1-like), histone H3K79 methyltransferase (S. cerevisiae), also known as DOT1L is a protein found in humans, as well as other eukaryotes. [The methylation of histone H3 lysine 79 (H3K79) by DOT1L which is a conserved epigenetic mark in many eukaryotic epigenomes, increases progressively along the aging process.

DOT1L inhibitors are chemical compounds that inhibits histone H3K79 methyltransferase.

Thus, “DOT1L inhibitor” refers to an agent capable of the decreasing the expression or enzymatic activity of DOT1L. For example, an EZH2 inhibitor results in a decrease in histone methylation of a target gene in a cell.

In certain embodiments, the DOT1L inhibitor decreases the expression or enzymatic activity of DOT1L by at least 5, 10, 20, 30, 40, 50, 60, 70, 80, 90, or 100% relative to a control, for example relative to a baseline level of activity.

In certain embodiments, the DOT1L inhibitor decreases histone methylation of a target gene by at least 5, 10, 20, 30, 40, 50, 60, 70, 80, 90, or 100% relative to a control, for example relative to a baseline level of activity.

In some embodiments, the DOT1L inhibitor increases expression or activity of a target gene by at least 5, 10, 20, 30, 40, 50, 60, 70, 80, 90, or 100% relative to a control, for example relative to a baseline level of activity.

In some embodiments, the DOT1L inhibitor decreases expression or enzymatic activity of DOT1L by at least about 1.1, 1.2, 1.3, 1.4, 1.5, 1.6, 1.7, 1.8, 1.9, 2, 3, 4, 5, 6, 7, 8, 9, 10, 15, 20, 30, 40, 50, 60, 70, 80, 90, 100, 200, 500, 1000-fold or more relative to a control, for example relative to a baseline level of activity.

In some embodiments, the DOT1L inhibitor decreases histone methylation of a target gene by at least about 1.1, 1.2, 1.3, 1.4, 1.5, 1.6, 1.7, 1.8, 1.9, 2, 3, 4, 5, 6, 7, 8, 9, 10, 15, 20, 30, 40, 50, 60, 70, 80, 90, 100, 200, 500, 1000-fold or more relative to a control, for example relative to a baseline level of activity.

In some embodiments, the DOT1L inhibitor increases expression or activity of a target gene by at least about 1.1, 1.2, 1.3, 1.4, 1.5, 1.6, 1.7, 1.8, 1.9, 2, 3, 4, 5, 6, 7, 8, 9, 10, 15, 20, 30, 40, 50, 60, 70, 80, 90, 100, 200, 500, 1000-fold or more relative to a control, for example relative to a baseline level of activity.

Exemplary DOT1L inhibitors are provided in Table 8.

TABLE 8

| | | pKi | | | | | | | Formulation | Human | |
|---|---|---|---|---|---|---|---|---|---|---|---|
| | | or | | Chemo- | Lit | | Lgr5+ | Perilymph | Conc. | In Vivo | Human |
| Agent | CAS | IC50 | | type | Cell | | Assay | Conc | Intratypm | Conc | Dosage |
| EPZ004777 | 1338466-77-5 | 0.3 | nM | Adenosine | 11 | nM | 0.6-45 μm | 0.6-45 μM | 0.1-45 mM | 0.1-45 uM | 10-100 |
| | | | | | | | | | | | mg/m2 |
| | | | | | | | | | | | per day |
| | | | | | | | | | | | IV |
| pinometostat | 1380288-87-8 | 0.08 | nM | Adenosine | 2.7 | nM | | 0.1-10 μM | 0.1-10 mM | Total | 54-90 |
| (EPZ-5676) | | | | | | | | | | plasma Css | mg/m2 |
| | | | | | | | | | | 800-1600 ng/mL | per day by |
| | | | | | | | | | | (1.42-2.94 uM) | continuous |
| | | | | | | | | | | (1-10 uM) | IV, |
| | | | | | | | | | | | Potential |
| | | | | | | | | | | | for SC |
| | | | | | | | | | | | dosing |
| SGC0946 | 1561178-17-3 | 0.3 | nM | Adenosine | 10 | mM | 0.6-5 μM | 0.6-5 μM | 0.6-5 mM | 0.1-5 μM | 10-100 |
| | | | | | | | | | | | mg/m2 |
| | | | | | | | | | | | per day |
| | | | | | | | | | | | IV |
| Bromo- | 1428254-21-0 | 77 | nM | Adenosine | | | | | | | |
| deaza- | | | | | | | | | | | |
| SAH | | | | | | | | | | | |
| CNSAH | 1985669-27-9 | 13 | nM | Adenosine | | | | | | | |
| Compound 10 | 1645266-99-4 | 29 | nM | Adenosine | | | | | | | |
| Compound 13 | 1940206-71-2 | 0.4 | nM | Amino- | | | | | | | |
| | | | | pyrimidine | | | | | | | |
| Compound 7 | 2088518-50-5 | <1 | nM | pyrrolo- | | | | | | | |
| | | | | pyrimidine | | | | | | | |
| Compound 8 | 1940224-84-9 | 14 | nM | Acetylene | | | | | | | |
| EPZ002696 | 1381760-94-6 | 13 | nM | Adenosine | | | | | | | |
| EPZ004450 | 1380315-97-8 | 4 | nM | Adenosine | | | | | | | |
| SAH | 979-92-0 | 600 | nM | Adenosine | | | | | | | |
| SYC-522 | 1381761-52-9 | 0.76 | mM | Adenosine | 6 | μM | | | | | |
| SYC-687 | 1440509-94-3 | 1.1 | mM | Non-Ribose | 200 | nM | | | | | |
| | 1440510-03-1, | 1.1 | nM | Adenosine | 200 | nM | | | | | |
| | 1440509-94-3 | | | | | | | | | | |
| Compound 21 | | | | Peptides | | | | | | | |
| Compound 28 | | | | Peptides | | | | | | | |
| Compound 6 | 167558-34-1 | 8.3 | μM | triazolo- | | | | | | | |
| | | | | thiadiazol | | | | | | | |
| Compound 8H | | | | pyrimidyl- | | | | | | | |
| | | | | aminoquinoline | | | | | | | |
| | 1163729-79-0 | 1.5 | μM | pyrimidine | | | | | | | |

In some embodiments the DOT1L inhibitor is EPZ004777, pinometostat or SGC0946.

### KDM Inhibitors

About 30 JmjC domain-containing proteins have been identified as lysine demethylases in the human genome. Based on histone lysine sites and demethylation states, the JmjC, domain-containing protein family is divided into six subfamilies: KDM2, KDM3, KDM4, KDM5 KDM6 and PHF. The JmjC, domain-containing proteins belong to the Fe(II) and 2-oxoglutarate (2-OG)-dependent dioxygenases, which demethylate a variety of targets, including histones (H3K4, H3K9, H3K27, H3K36 as well as H1K26) and non-histone proteins. Unlike the LSD family, the JmjC-domain-containing histone demethylases (JHDMs) are able to erase all three kinds of histone lysine-methylation states since the JHDMs do not require protonated nitrogen for demethylation.

The KDM2 (also named FBXL) subfamily includes two members: KDM2A and KDM2B. KDM4 gene family, first identified in silico, consists of six members, including KDM4A, KDM4B, KDM4C, KDM4D, KDM4E and KDM4F. The KDM5 subfamily contains four enzymes: KDM5A, KDM5B, KDM5C and KDM5D, which specifically remove methyl marks from H3K4me2/3. In the human genome, the KDM6 subfamily is comprised of KDM6A, KDM6B and UTY, which share a well-conserved JmjC histone catalytic domain.

KDM inhibitors are chemical compounds that inhibits lysine demethylases.

Thus, “KDM inhibitor” refers to an agent capable of the decreasing the expression or enzymatic activity of KDM. For example, an KDM inhibitor results in a decrease in histone demethylation of a target gene in a cell.

In certain embodiments, the KDM inhibitor decreases the expression or enzymatic activity of KDM by at least 5, 10, 20, 30, 40, 50, 60, 70, 80, 90, or 100% relative to a control, for example relative to a baseline level of activity.

In certain embodiments, the KDM inhibitor decreases histone demethylation of a target gene by at least 5, 10, 20, 30, 40, 50, 60, 70, 80, 90, or 100% relative to a control, for example relative to a baseline level of activity.

In some embodiments, the KDM inhibitor increases expression or activity of a target gene by at least 5, 10, 20, 30, 40, 50, 60, 70, 80, 90, or 100% relative to a control, for example relative to a baseline level of activity.

In some embodiments, the KDM inhibitor decreases expression or enzymatic activity of KDM by at least about 1.1, 1.2, 1.3, 1.4, 1.5, 1.6, 1.7, 1.8, 1.9, 2, 3, 4, 5, 6, 7, 8, 9, 10, 15, 20, 30, 40, 50, 60, 70, 80, 90, 100, 200, 500, 1000-fold or more relative to a control, for example relative to a baseline level of activity.

In some embodiments, the KDM inhibitor decreases histone demethylation of a target gene by at least about 1.1, 1.2, 1.3, 1.4, 1.5, 1.6, 1.7, 1.8, 1.9, 2, 3, 4, 5, 6, 7, 8, 9, 10, 15, 20, 30, 40, 50, 60, 70, 80, 90, 100, 200, 500, 1000-fold or more relative to a control, for example relative to a baseline level of activity.

In some embodiments, the KDM inhibitor increases expression or activity of a target gene by at least about 1.1, 1.2, 1.3, 1.4, 1.5, 1.6, 1.7, 1.8, 1.9, 2, 3, 4, 5, 6, 7, 8, 9, 10, 15, 20, 30, 40, 50, 60, 70, 80, 90, 100, 200, 500, 1000-fold or more relative to a control, for example relative to a baseline level of activity.

Exemplary KDM inhibitors are provided in Table 9.

TABLE 9

| | | | Covalent | Select | Select | Select | Select | | Select | | Select |
|---|---|---|---|---|---|---|---|---|---|---|---|
| Agent | CAS | Chemo-type | or not | KDM1 | KDM2 | KDM3 | KDM4 | | KDM5 | | KDM6 |
| AS 8351 | 796-42-9 | Hydrzone | | | | | | | | | |
| TC-E | 1453071-47-0 | Hydroamate | No | | 6.8 μM | | 83 | μM | 55 | μM | >100 μM |
| 5002 | | | | | | | | | | | |
| EPT-103182 | | | | | | | 20-50 | | <100 | nM | 3K |
| Compound 54k | 1844064-06-7 | pyrimidin-4-one | No | | | | 102/31 | nM | 23 | nM | |
| Cmpd 1 | 1546899-38-9 | Cyproamine | Yes | | 220 nM | | | | 190 | nM | |
| | | isonicotinic acid | | | | | | | | | |
| Cmpd 105 | 1613514-89-8 | Isonicotinic acid | No | | | | | | <100 | nM | |
| Compound 34 | 1461602-86-7 | Isonicotinic acid | No | | | | <100 | nM | <100 | nM | |
| Compound 41 | 1628332-52-4 | pyridopyrimidinone | No | | | | | | <100 | nM | |
| Compound 48 | 16282.10-26-3 | cyanopyrazole | No | | | | | | 15 | nM | |
| Compound 18 | 1993438-65-5 | naphthyridones | No | | | | | | 206 | nM | |
| Compound 33 | 1613410-75-5 | pyrazolylpyridines | No | | | | | | 10 | nM | |
| Compound 48 | 1905482-57-6 | Amide | No | | | | | | | | |
| Compound 48 | 1905482-57-6 | Pyrazole | No | | | | | | | | |
| Compound 49 | 1905481-35-7 | Pyrazole | No | | | | | | | | |
| Compound 50 | 1905481-36-8 | Pyrazole | No | | | | | | | | |
| Compound 6 | 2169272-46-0 | 1-H-Indole | No | | | | | | 50 | nM | |
| Compound R-35 | 1807514-47-1 | Triazole | No | | | | | | 65 | nM | |
| CPI-455 | 1628208-23-0 | cyanopyiazole | No | | | | 41 | nM | 13.2 | nM | |
| CPI-4203 | 1628214-07-2 | cyanopyrazol | No | | | | | | 1.1 | μM | |
| E67-2 | 1364914-62-4 | Quinazoline | No | | | | | | | | |
| GSK-467 | 1628332-52-4 | Pyrazole | No | | | | | | 14 | nM | |
| GSK-J1 | 1373422-53-7 | Acid | No | | | | | | | | |
| GSK-J4 | 1373423-53-0 | Ethyl Ester | No | | | | | | 170 | nM | 28 nM |
| KDM5-C49 | 1596348-16-1 | Pyridine | No | | | | | | | | |
| KDM5-C50 | 1596348-32-1 | Pyridine | No | | | | | | | | |
| KDOAM25 | 2230731-99-2 | Amide | No | | | | | | | | |
| N11 | 1613515-45-9 | isonicotinic | No | | | | | | 65 | nM | |
| | 1807514-47-1 | Amide | No | | | | | | 90 | nM | |
| | 1844064-07-8 | Pyridopyrimidinone | No | | | | | | 45 | nM | |
| Compound 1 | | Rh Complex | | | | | | | | | |
| Compound 15e | 1498996-89-6 | Hydrazine | | | | | | | X | | |
| Daminozide | 1596-84-5 | Hydrazine | | | X | | | | | | |
| JIB-04 | 99596-05-9 | Hydrazine | | | | | | | 220 | μM | |
| Methylstat | 1310877-95-2 | Unsat amide | | | | | | | | | |
| Compound 10r | 2098902-68-0 | cyanopyrazole | No | | | | | | | | |
| N71 | | | Yes | | | | X | | | | |
| NSC | | | | | | | 410 | nM | | | |
| 6369819 | | | | | | | | | | | |

| | | | | | | | | | | | Human | | |
|---|---|---|---|---|---|---|---|---|---|---|---|---|---|
| | Select | Select | Lit | | Lgr5+ | | Perilymph | | Formulation | | In Vivo | | Human |
| Agent | KDM7 | KDM8 | Cell | | Assay | | Conc | | Conc | | Conc | | Dosage |
| AS 8351 | | | | | 1-3 | μM | 1-3 | μM | 1-3 | mM | 1-3 | mM | 100-2000 |
| | | | | | | | | | | | | | mg/day |
| TC-E | 0.2, | | 16-40 | μM | 0.12-10 | μM | 0.12-10 | μM | 0.12-10 | mM | 0.12-10 | μM | 100-1000 |
| 5002 | 1, 2 μM | | | | | | | | | | | | mg/day |
| EPT-103182 | | | 1.8 | nM | 1-100 | nM | 1-100 | nM | 1-100 | μM | 5-50 | nM | 10 mg to |
| | | | | | | | | | | | | | 1000 mg/day |
| Compound 54k | | | | | | | | | | | | | |
| Cmpd 1 | 220 nM | | | | | | | | | | | | |
| Cmpd 105 | | | 0.1-1 | μM | | | | | | | | | |
| Compound 34 | | | | | | | | | | | | | |
| Compound 41 | | | | | | | | | | | | | |
| Compound 48 | | | 340 | nM | | | | | | | | | |
| Compound 18 | | | >10 | μM | | | | | | | | | |
| Compound 33 | | | ~1 | μM | | | | | | | | | |
| Compound 48 | | | 1-10 | μM | | | | | | | | | |
| Compound 48 | | | | | | | | | | | | | |
| Compound 49 | | | | | | | | | | | | | |
| Compound 50 | | | | | | | | | | | | | |
| Compound 6 | | | | | | | | | | | | | |
| Compound R-35 | | | 1.5 | μM | | | | | | | | | |
| CP1-455 | | | 90 | nM | | | | | | | | | |
| CPI-4203 | | | | | | | | | | | | | |
| E67-2 | | | | | | | | | | | | | |
| GSK-467 | | | | | | | | | | | | | |
| GSK-J1 | | | 50 | μM | | | | | | | | | |
| GSK-J4 | | | | | | | | | | | | | |
| KDM5-C49 | | | | | | | | | | | | | |
| KDM5-C50 | | | | | | | | | | | | | |
| KDOAM25 | | | | | | | | | | | | | |
| N11 | | | 1.6 | μM | | | | | | | | | |
| | | | 960 | μM | | | | | | | | | |
| Compound 1 | | | | | | | | | | | | | |
| Compound 15e | | | | | | | | | | | | | |
| Daminozide | X | | | | | | | | | | | | |
| JIB-04 | | | | | | | | | | | | | |
| Methyl slat | | | | | | | | | | | | | |
| Compound 10r | | | | | | | | | | | | | |
| N71 | | | | | | | | | | | | | |
| NSC | | | | | | | | | | | | | |
| 6369819 | | | | | | | | | | | | | |

In some embodiments the KDM inhibitor is AS 8351 or TC-E 5002.

### Wnt Agonists

A Wnt agonist refers to an agent that increases the expression, levels, and/or activity of a Wnt gene, protein, or signaling pathway (e.g. TCF/LEF, Frizzled receptor family, Wif1, Lef1, Axin2, β-catenin) in a cell, for example, a cochlear cell. A Wnt agonist includes a GSK3 inhibitor, such as a GSK3-α or a GSK3-β inhibitor. In some embodiments, the GSK3 inhibitor is a GSK3-β inhibitor.

The TCF/LEF family is a group of transcription factors that bind to DNA through a high mobility group domain, and which are involved in the Wnt signaling pathway where they recruit the coactivator β-catenin to enhancer elements of targeted genes. Frizzled is a family of G protein-coupled receptor proteins that serves as receptors in the Wnt signaling pathway. Frizzled receptors inhibit intracellular β-catenin degradation and activate TCF/LEF-mediated transcription.

In some embodiments, the Wnt agonist increases Wnt signaling in a cochlear or vestibular cell by about or at least about 10, 20, 30, 40, 50, 60, 70, 80, 90, 100, 200, 300, 400, or 500% or more (or at least about 1.1, 1.2, 1.3, 1.4, 1.5, 1.6, 1.7, 1.8, 1.9, 2, 3, 4, 5, 6, 7, 8, 9, 10, 15, 20, 30, 40, 50, 60, 70, 80, 90, 100, 200, 500, 1000-fold or more) or more relative to a control, for example relative to a baseline level of activity.

In some embodiments, the Wnt agonist increases TCF/LEF-mediated transcription in a cochlear or vestibular cell, for example, by about or at least about 10, 20, 30, 40, 50, 60, 70, 80, 90, 100, 200, 300, 400, or 500% or more (or at least about 1.1, 1.2, 1.3, 1.4, 1.5, 1.6, 1.7, 1.8, 1.9, 2, 3, 4, 5, 6, 7, 8, 9, 10, 15, 20, 30, 40, 50, 60, 70, 80, 90, 100, 200, 500, 1000-fold or more) or more relative to a control, for example relative to a baseline level of activity.

In some embodiments, the Wnt agonist binds and activates a Frizzled receptor family member, for example, by about or at least about 10, 20, 30, 40, 50, 60, 70, 80, 90, 100, 200, 300, 400, or 500% or more (or at least about 1.1, 1.2, 1.3, 1.4, 1.5, 1.6, 1.7, 1.8, 1.9, 2, 3, 4, 5, 6, 7, 8, 9, 10, 15, 20, 30, 40, 50, 60, 70, 80, 90, 100, 200, 500, 1000-fold or more) or more relative to a control, for example relative to a baseline level of activity.

In some embodiments, the Wnt agonist inhibits GSK3 for example, by about or at least about 10, 20, 30, 40, 50, 60, 70, 80, 90, 100, 200, 300, 400, or 500% or more (or at least about 1.1, 1.2, 1.3, 1.4, 1.5, 1.6, 1.7, 1.8, 1.9, 2, 3, 4, 5, 6, 7, 8, 9, 10, 15, 20, 30, 40, 50, 60, 70, 80, 90, 100, 200, 500, 1000-fold or more or more relative to a control, for example relative to a baseline level of activity.

In some embodiments, the Wnt agonist preferentially upregulates Jag-1, Deltex-1 or Hif-1 more than the Wnt agonist upregulates Hes or Hey. In some embodiments, the Wnt agonist increases the expression of Jag-1, Deltex-1 and/or Hif-1 10%, 25%, 50%, 75%, 100%, 125%, 150%, 175%, 200%, 250% or more than it increases the expression or activity of Hes and Hey.

Exemplary agents having activity as a Wnt agonist are provided in Table 2 and 3 below below, including pharmaceutically-acceptable salts thereof.

TABLE 2

| | | | | | | | | Formul. | |
|---|---|---|---|---|---|---|---|---|---|
| | | GSK-3 | GSK-3 | Lgr5+ | | Perilymph | | Conc. | |
| Agent | CAS | alpha | alpha | Assay | | Conc. | | Intraymp | |
| CHIR99021 | 252917-06-9 | 4.4 nM | 6.6 nM | 2-6 | μM | 2-6 | μM | 4 | mM |
| AZD 1080 | 612487-72-6 | 6.9 nM | 31 nM | 1-5 | μM | 1-5 | μM | 1-5 | mM |
| GSK XX1I | 1195901-31-5 | 2.3 nM | 2.0 nM | 0.2-1 | μM | 0.2-1 | μM | 0.2-1 | mM |
| LY2090314 | 603288-22-8 | 2.1 nM | 0.9 nM | 5-20 | nM | 5-20 | nM | 5-20 | μM |

TABLE 3

| Class | Agent | CAS |
|---|---|---|
| WNT | | |
| ARFGAP1 | QS 11 | 944328-88-5 |
| ARFGAP1 | WASP-1, ZINC00087877 | 352328-82-6 |
| Axin | Cpd1 | 1357473-75-6 |
| Axin | Cpd2 | 1228659-47-9 |
| Axin | HLY78 | 854847-61-3 |
| Axin | SKL2001 | 909089-13-0 |
| beta-catenin | DCA | 56-47-3 |
| Disrupts the Axin | Compound 2 | 1360540-82-4 |
| Complex | | |
| Disrupts the Axin | Compound 71 | 1622429-71-3 |
| Complex | | |
| Disrupts the Axin | ISX 9 | 832115-62-5 |
| Complex | | |
| DKK1 inhibitor | WAY-262611 | 1123231-07-1 |
| MEK | Radicicol | 12772-57-5 |
| MEK | Selumetinib (AZD6244) | 606143-52-6 |
| PP2A | IQ 1 | 331001-62-8 |
| sFRP-1 inhibitor | (Dimethylamino)propyl)-2-ethyl-5- | 915754-88-0 |
| | (phenylsulfonyl)benzenesulfonamide | |
| sFRP-1 inhibitor | Cyclosporine A (CsA) | 59865-13-3 |
| sFRP-1 inhibitor | Cyclosporine analogs | |
| sFRP-1 inhibitor | PSC833 (Valspodar) | 121584-18-7 |
| sFRP-1 inhibitor | WAY 316606 | 915759-45-4 |
| Target Undetermined | Diketones | WO 2016029021 A1; |
| | | WO 2012024404 A1 |
| Target Undetermined | Diketones | 1622429-56-4 |
| Target Undetermined | Diketones | 1360540-88-0 |
| Target Undetermined | Diketones | 1360540-89-1 |
| Target Undetermined | Diketones | 1622429-79-1 |
| Target Undetermined | Diketones | 1622429-75-7 |
| Target Undetermined | Diketones | 1622429-74-6 |
| Target Undetermined | Diketones | 1622430-76-5 |
| Target Undetermined | Diketones | 1622430-31-2 |
| Target Undetermined | Diketones | 1622430-52-7 |
| Target Undetermined | Diketones | 1622429-67-7 |
| Target Undetermined | Diketones | 1622429-65-5 |
| Target Undetermined | Diketones | 1622429-69-9 |
| van-Gogh-like receptor | Compound 109 | 1314885-81-8 |
| proteins (Vangl) | | |
| Wnt Ligand | Wnt-1 | Protein |
| Wnt Ligand | Wnt-10a | Protein |
| Wnt Ligand | Wnt-10b/12 | Protein |
| Wnt Ligand | Wnt-11 | Protein |
| Wnt Ligand | Wnt-16 | Protein |
| Wnt Ligand | Wnt-2/Irp (Int-I-related protein) | Protein |
| Wnt Ligand | Wnt-2b/13 | Protein |
| Wnt Ligand | Wnt-3/Int-4 | Protein |
| Wnt Ligand | Wnt-3a | Protein |
| Wnt Ligand | Wnt-4 | Protein |
| Wnt Ligand | Wnt-5a | Protein |
| Wnt Ligand | Wnt-5b | Protein |
| Wnt Ligand | Wnt-6 | Protein |
| Wnt Ligand | Wnt-7a | Protein |
| Wnt Ligand | Wnt-7b | Protein |
| Wnt Ligand | Wnt-8a/8d | Protein |
| Wnt Ligand | Wnt-8b | Protein |
| Wnt Ligand | Wnt-9a/14 | Protein |
| Wnt Ligand | Wnt-9b/14b/15 | Protein |
| Wnt Related Protein | Norrin | Protein |
| Wnt Related Protein | R-Spondin 1/2/3/4 | Protein |
| Wnt-3a/Dkk-1 | BML-284 | 853220-52-7 |
| Wnt-3a/Dkk-1 | Compound 1 | 1084833-94-2 |
| Wnt-3a/Dkk-1 | Compound 25 | 1084834-05-8 |
| GSK3 alpha | | |
| CREB knockdown | 666-15 | 1433286-70-4 |
| Isonicotinamides | Compound 29 | 1772823-37-6 |
| Isonicotinamides | Compound 33 | 1772823-64-9 |
| Isonicotinamides | Compound 39 | 1772824-10-8 |
| Maleimide | I5 | 264217-24-5 |
| Maleimide | Tivantinib | 905854-02-6 |
| Organometallic | Compound (R)-DW12 | 1047684-07-0 |
| Organometallic | Compound 3 | 1498285-39-4 |
| | | 1498285-48-5 |
| Organometallic | Compound lambda-OS1 | 1291104-51-2 |
| | | 1292843-11-8 |
| Oxadiazoles | Compound 14d | 1374671-64-3 |
| Oxadiazoles | Compound 15b | 1374671-66-5 |
| Oxadiazoles | Compound 27 | 1820758-44-8 |
| Oxindole | AZD1080 | 612487-72-6 |
| Pyrazole | AT 7519 | 844442-38-2 |
| Pyrazole | Compound 4a | 1627557-91-8 |
| Pyrazole | Compound 4t | 1627558-10-4 |
| Pyrazole | Compound 4z | 1627558-16-0 |
| Pyrazole | GSK-3b XXII | 1195901-31-5 |
| Pyrazolopyridazines | Compound 18 | 405223-20-3 |
| Pyrazolopyridazines | Compound 19 | 405223-71-4 |
| Pyrazolopyridines | Compound 14 | 583038-63-5 |
| Pyrazolopyridines | Compound 23 | 583038-76-0 |
| Pyrazolopyridines | Pyrazolopyridine 34 | 583039-27-4 |
| Pyrazolo- | BRD1172 | 1597438-86-2 |
| tetrahydroquinolinone | | |
| Pyrazolo- | BRD1652 | 1597438-93-1 |
| tetrahydroquinolinone | | |
| Pyrazolo- | BRD4003 chiral | 1597439-60-5 |
| tetrahydroquinolinone | | |
| Pyrazolo- | BRD4003 chiral | 1597439-59-2 |
| tetrahydroquinolinone | | |
| Pyrazolo- | Compound 11 | 1597439-12-7 |
| tetrahydroquinolinone | | |
| Pyrazolo- | Compound 16 | 1597440-17-9 |
| tetrahydroquinolinone | | |
| Pyrazolo- | Compound 8 | 1597439-01-4 |
| tetrahydroquinolinone | | |
| Pyrazolo- | Compound 9 | 1597439-02-5 |
| tetrahydroquinolinone | | |
| Triazolpyrimidine | Compound 90 | 91322-11-1 |
| Triazolpyrimidine | Compound 92 | 1043429-30-6 |
| Urea | AR-A014418 | 487021-52-3 |
| GSK3-beta | | |
| Acid | Bikinin | 188011-69-0 |
| Acid | Valproic Acid, Sodium Salt | 99-66-1 |
| Aloisines | Aloisine A | 496864-16-5 |
| Aloisines | Aloisine B | 496864-14-3 |
| Aloisines | TWS119 | 1507095-58-0 |
| Aminopyrimidine | CHIR98014 (CT98014) | 252935-94-7 |
| Aminopyrimidine | CHIR98023 (CT98023) | 252904-84-0 |
| Aminopyrimidine | CHIR98024 (CT98024) | 556813-39-9 |
| Aminopyrimidine | CHIR99021 (CT99021) | 252917-06-9 |
| Aminopyrimidine | CT20026 | 403808-63-9 |
| Aminopyrimidinyl | CGP60474 | 164658-13-3 |
| Aminopyrimidinyl | GSK-3β Inhibitor XVIII | 1139875-74-3 |
| Azaindolylmaleimide | Compound 29 | 436866-61-4 |
| Azaindolylmaleimide | Compound 46 | 682807-74-5 |
| Bisindolylmaleimide | Bisindolylmaleimide X HCl | 131848-97-0 |
| Bisindolylmaleimide | Compound 5a | 436866-54-5 |
| Bisindolylmaleimide | Enzastaurin (LY317615) | 170364-57-5 |
| Bisindolylmaleimide | GF109203x | 176504-36-2 |
| Bisindolylmaleimide | Ro318220 | 125314-64-9 |
| Dihydropyridine | ML320 | 1597438-84-0 |
| Flavone | Flavopiridol | 146426-40-6 |
| Furanosesquiterpenes | Palinurin | 254901-27-4 |
| Furanosesquiterpenes | Tricantin | 853885-55-9 |
| Furopyrimidine | Compound 100 | 744255-19-4 |
| Halomethylketones | Compound 17 | 62673-69-2 |
| Halomethylketones | GSK-3β Inhibitor VI | 62673-69-2 |
| Halomethylketones | GSK-3β Inhibitor VII | 99-73-0 |
| Hymenidin | Hymenidin | 107019-95-4 |
| Indirubins | 5-Iodo-indirubin-3′-monoxime | 331467-03-9 |
| Indirubins | 6-Bromoindirubin-3-acetoxime | 667463-85-6 |
| Indirubins | GSK-3 Inhibitor IX | 667463-62-9 |
| Indirubins | GSK-3 Inhibitor X | 740841-15-0 |
| Indirubins | Indirubin | 479-41-4 |
| Indirubins | Indirubin-3'-monoxime | 160807-49-8 |
| Indirubins | Indirubin-5-sulfonic acid sodium salt | 331467-05-1 |
| Inorganic atom | Beryllium | |
| Inorganic atom | Lithium Chloride | |
| Inorganic atom | Tungstate | |
| Inorganic atom | Zinc | |
| Isoindolone | Staurosporine | 62996-74-1 |
| Isonicotinamides | Compound 29 | 1772823-37-6 |
| Isonicotinamides | Compound 33 | 1772823-64-9 |
| Isonicotinamides | Compound 39 | 1772824-10-8 |
| Maleimide | 3F8 | 159109-11-2 |
| Maleimide | 603281-31-8 | 603281-31-8 |
| Maleimide | BIP-135 | 941575-71-9 |
| Maleimide | Compound 34 | 396091-16-0 |
| Maleimide | CP21R7 | 125314-13-8 |
| Maleimide | GSK-3 inhibitor 1 | 603272-51-1 |
| Maleimide | GSK-3β Inhibitor XI | 626604-39-5 |
| Maleimide | I5 | 264217-24-5 |
| Maleimide | IM-12 | 1129669-05-1 |
| Maleimide | Isogranulatimide | 244148-46-7 |
| Maleimide | KT 5720 | 108068-98-0 |
| Maleimide | LY2090314 | 603288-22-8 |
| Maleimide | SB-216763 | 280744-09-4 |
| Maleimide | SB-415286 (SB-41528) | 264218-23-7 |
| Maleimide | TCS 21311 | 1260181-14-3 |
| Maleimide | Tivantinib | 905854-02-6 |
| Manzamines | Manzamine A | 104196-68-1 |
| Miscellaneous | AZD2858 (AR28) | 486424-20-8 |
| Miscellaneous | CID 755673 | 521937-07-5 |
| Miscellaneous | Dibromocantharelline | 101481-34-9 |
| Miscellaneous | TCS 2002 | 1005201-24-0 |
| Organometallic | (RRu)-HB1229 | |
| Organometallic | (RRu)-NP549 | |
| Organometallic | Compound (R)-DW12 | 1047684-07-0 |
| Organometallic | Compound 3 | 1498285-39-4, |
| | | 1498285-48-5 |
| Organometallic | Compound lambda-OS1 | 1291104-51-2, |
| | | 1292843-11-8 |
| Organometallic | DW12 | 861251-33-4 |
| Organometallic | HB12 | 800384-87-6 |
| Organometallic | NP309 | 937810-13-4 |
| Oxadiazol | Compound 14d | 1374671-64-3 |
| Oxadiazol | Compound 15b | 1374671-66-5 |
| Oxadiazol | Compound 20x | 1005201-80-8 |
| Oxadiazol | GSK-3 Inhibitor II | 478482-75-6 |
| Oxadiazol | GSK3 Inhibitor, 2 | 1377154-01-2 |
| Oxadiazol | TC-G 24 | 1257256-44-2 |
| Oxindole | AZD1080 | 612487-72-6 |
| Oxindole | SU9516 | 77090-84-1 |
| Patent | CN 101341138 B | |
| Patent | CN 1319968 C | |
| Patent | CP-70949 | |
| Patent | CT118637 | |
| Patent | EP 1739087 A1 | |
| Patent | EP 1961748 A2 | |
| Patent | EP 2765188 A1 | |
| Patent | GI179186X | |
| Patent | GW784752X | |
| Patent | GW784775X | |
| Patent | US 20070088080 A1 | |
| Patent | US 20100292205 A1 | |
| Patent | U.S. Pat. No. 7,514,445 B2 | |
| Patent | U.S. Pat. No. 8,071,591 B2 | |
| Patent | U.S. Pat. No. 8,207,216 B2 | |
| Patent | U.S. Pat. No. 8,686,042 B2 | |
| Patent | U.S. Pat. No. 8,771,754 B2 | |
| Patent | WO 2001085685 A1 | |
| Patent | WO 2003037891 A1 | |
| Patent | WO 2006018633 A1 | |
| Patent | WO 2007102770 A1 | |
| Patent | WO 2008077138 A1 | |
| Patent | WO 2009017453 A1 | |
| Patent | WO 2010075551 A1 | |
| Patent | WO 2010104205 A1 | |
| Patent | WO 2011089416 A1 | |
| Patent | WO 2013124413 A1 | |
| Patent | WO 2014003098 A1 | |
| Patent | WO 2014013255 A1 | |
| Patent | WO 2014050779 A1 | |
| Patent | WO 2014059383 A1 | |
| Patent | WO 2014083132 A1 | |
| Patent | WO2006100490A1/EP 1863904 A1 | |
| Patent | WO2009017455 A1 | |
| Paullone | Cmpd 17b | 408532-42-3 |
| Paullone | Kenpaullone | 142273-20-9 |
| Paullones | Alsterpaullone | 237430-03-4 |
| Paullones | Alsterpaullone CN Ethyl | 852529-97-0 |
| Paullones | Azakenpaullone | 676596-65-9 |
| Paullones | Cazpaullone | 914088-64-5 |
| Peptide | FRATtide | |
| Peptide | L803 | |
| Peptides | L803-mts | |
| Publication | 705701 | |
| Publication | 708244 | |
| Publication | 709125 | |
| Publication | AR79 | |
| Publication | AZ13282107 | No Structure |
| Publication | AZ13282107 | |
| Publication | CEP-16805 | No Structure |
| Publication | CG-301338 | No Structure |
| Publication | CT73911 | |
| Publication | LY2064827 | |
| Publication | NP-103 | No Structure |
| Publication | SAR 502250 | No Structure |
| Publication | SAR 502250 (Sanofi) | 1073653-58-3 |
| Publication | XD-4241 | No Structure |
| Pyrazole | AT 7519 | 844442-38-2 |
| Pyrazole | Compound 4a | 1627557-91-8 |
| Pyrazole | Compound 4t | 1627558-10-4 |
| Pyrazole | Compound 4z | 1627558-16-0 |
| Pyrazole | GSK-3 Inhibitor XXII | 1195901-31-5 |
| Pyrazolone | GSK-3beta Inhibitor XXVI | 871843-09-3 |
| Pyrazolopyridazines | Compound 18 | 405223-20-3 |
| Pyrazolopyridazines | Compound 19 | 405223-71-4 |
| Pyrazolopyridine | Pyrazolopyridine 18 | 405221-39-8 |
| Pyrazolopyridine | Pyrazolopyridine 34 | 583039-27-4 |
| Pyrazolopyridine | Pyrazolopyridine 9 | 923029-74-7 |
| Pyrazolopyridines | Compound 14 | 583038-63-5 |
| Pyrazolopyridines | Compound 14 | 583038-63-5 |
| Pyrazolopyridines | Compound 23 | 583038-76-0 |
| Pyrazoloquinoxaline | NSC 693868 (Compound 1) | 40254-90-8 |
| Pyrazoloquinoxaline | NSC 693868 (Compound 1) | 40254-90-8 |
| Pyridinone | Compound 150 | 1282042-18-5 |
| Pyrrolopyridinyl | Compound 12 | 2025388-10-5 |
| Pyrrolopyridinyl | Compound 27 | 2025388-25-2 |
| Pyrroloazepine | Hymenialdisine | 82005-12-7 |
| Quinazolin | GSK-3 Inhibitor XIII | 404828-08-6 |
| Quinolinecarb | VP0.7 | 331963-23-6 |
| Quinolinecarboxamide | | 1132813-46-7 |
| Quinolinecarboxamide | | 1132812-98-6 |
| Quinolinecarboxamide | | 950727-66-9 |
| Thiadiazolidindiones | GSK-3β Inhibitor I | 327036-89-5 |
| Thiadiazolidindiones | NP031112 (Tideglusib) | 865854-05-3 |
| Thiadiazolidindiones | NP031115 | 1400575-57-6 |
| Triazolpyrimidine | Compound 90 | 91322-11-1 |
| Triazolpyrimidine | Compound 92 | 1043429-30-6 |
| Urea | GSK-3β Inh. VIII AR-A014418 | 487021-52-3 |
| Urea | A-1070722 | 1384424-80-9 |

In some embodiments, an agent of having activity as a Wnt agonist is a GSK3 inhibitor. In some embodiments, the GSK3 inhibitor is AZD1080, GSK3 inhibitor XXII, CHIR99021 or LY2090314. In one embodiment, the Wnt agonist is CHIR99021. In other embodiments, Wnt agonist and/or GSK3 inhibitor is a substituted 3-Imidazo[1,2-a]pyridin-3-yl-4-(1,2,3,4-tetrahydro-[1,4]diazepino-[6,7,1-hi]indol-7-yl)pyrrole-2,5-dione. (Formula A.)

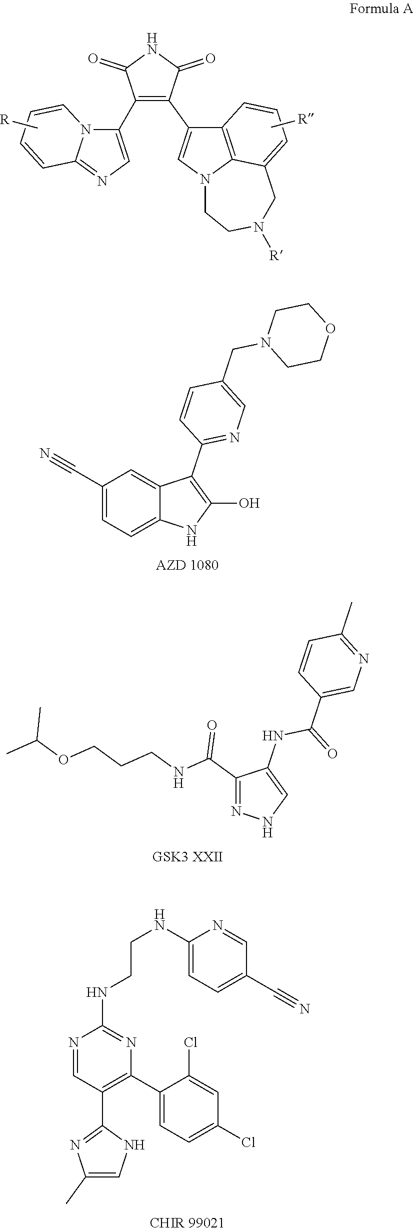

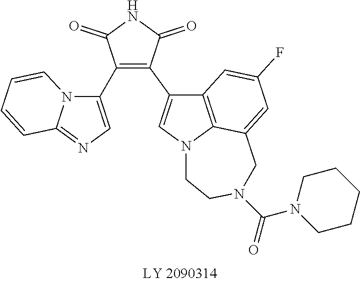 [SMILES: O=C1NC(=O)C(c2cnc3ccccn23)=C1c1cn2c3c(cc(F)cc13)CN(C(=O)N1CCCCC1)CC2]

The Wnt agonist can be any selected from WO 2018/125746, which is hereby incorporated by reference. In some embodiments, the Wnt agonist can be the compound as defined in claim 1 of WO 2018/125746. In some embodiments, the Wnt agonist can be the compound as defined in claim 12 of WO 2018/125746.”

Exemplary, substituted 3-Imidazo[1,2-a]pyridin-3-yl-4-(1,2,3,4-tetrahydro-[1,4]diazepino-[6,7,1-hi]indol-7-yl)pyrrole-2,5-dione include: 3-(imidazo[1,2-a]pyridin-3-yl)-4-(2-(piperidine-1-carbonyl)-9-(trifluoromethyl)-1,2,3,4-tetrahydro-[1,4]diazepino[6,7,1-hi]indol-7-yl)-1H-pyrrole-2,5-dione; 7-(4-(imidazo[1,2-a]pyridin-3-yl)-2,5-dioxo-2,5-dihydro-1H-pyrrol-3-yl)-2-(piperidine-1-carbonyl)-1,2,3,4-tetrahydro-[1,4]diazepino[6,7,1-hi]indole-9-carbonitrile; 3-(9-ethynyl-2-(piperidine-1-carbonyl)-1,2,3,4-tetrahydro-[1,4]diazepino[6,7,1-hi]indol-7-yl)-4-(imidazo[1,2-a]pyridin-3-yl)-1H-pyrrole-2,5-dione; 3-(9-amino-2-(piperidine-1-carbonyl)-1,2,3,4-tetrahydro-[1,4]diazepino[6,7,1-hi]indol-7-yl)-4-(imidazo[1,2-a]pyridin-3-yl)-1H-pyrrole-2,5-dione; 1-(9-fluoro-7-(4-(imidazo[1,2-a]pyridin-3-yl)-2,5-dioxo-2,5-dihydro-1H-pyrrol-3-yl)-1,2,3,4-tetrahydro-[1,4]diazepino[6,7,1-hi]indole-2-carbonyl)piperidine-4-carbaldehyde; 3-(9-fluoro-2-(4-(hydroxymethyl)piperidine-1-carbonyl)-1,2,3,4-tetrahydro-[1,4]diazepino[6,7,1-hi]indol-7-yl)-4-(imidazo[1,2-a]pyridin-3-yl)-1H-pyrrole-2,5-dione; 3-(2-(4,4-difluoropiperidine-1-carbonyl)-9-fluoro-1,2,3,4-tetrahydro-[1,4]diazepino[6,7,1-hi]indol-7-yl)-4-(imidazo[1,2-a]pyridin-3-yl)-1H-pyrrole-2,5-dione; 3-(2-(8-oxa-3-azabicyclo[3.2.1]octane-3-carbonyl)-9-fluoro-1,2,3,4-tetrahydro-[1,4]diazepino[6,7,1-hi]indol-7-yl)-4-(imidazo[1,2-a]pyridin-3-yl)-1H-pyrrole-2,5-dione; 3-(benzo[d]isoxazol-3-yl)-4-(9-fluoro-2-(piperidine-1-carbonyl)-1,2,3,4-tetrahydro-[1,4]diazepino[6,7,1-hi]indol-7-yl)-1H-pyrrole-2,5-dione; N-(7-(4-(imidazo[1,2-a]pyridin-3-yl)-2,5-dioxo-2,5-dihydro-1H-pyrrol-3-yl)-2-(piperidine-1-carbonyl)-1,2,3,4-tetrahydro-[1,4]diazepino[6,7,1-hi]indol-9-yl)acetamide; 3-(9-(difluoromethyl)-2-(piperidine-1-carbonyl)-1,2,3,4-tetrahydro-[1,4]diazepino[6,7,1-hi]indol-7-yl)-4-(imidazo[1,2-a]pyridin-3-yl)-1H-pyrrole-2,5-dione; 3-(2-(3,3-difluoropiperidine-1-carbonyl)-9-fluoro-1,2,3,4-tetrahydro-[1,4]diazepino[6,7,1-hi]indol-7-yl)-4-(imidazo[1,2-a]pyridin-3-yl)-1H-pyrrole-2,5-dione; 3-(2-((1R,4R)-2,5-diazabicyclo[2.2.1]heptane-2-carbonyl)-9-fluoro-1,2,3,4-tetrahydro-[1,4]diazepino[6,7,1-hi]indol-7-yl)-4-(imidazo[1,2-a]pyridin-3-yl)-1H-pyrrole-2,5-dione; 2-(8-oxa-3-azabicyclo[3.2.1]octane-3-carbonyl)-7-(4-(imidazo[1,2-a]pyridin-3-yl)-2,5-dioxo-2,5-dihydro-1H-pyrrol-3-yl)-1,2,3,4-tetrahydro-[1,4]diazepino[6,7,1-hi]indole-9-carbonitrile; 2-(3,3-difluoropiperidine-1-carbonyl)-7-(4-(imidazo[1,2-a]pyridin-3-yl)-2,5-dioxo-2,5-dihydro-1H-pyrrol-3-yl)-1,2,3,4-tetrahydro-[1,4]diazepino[6,7,1-hi]indole-9-carbonitrile; 2-(4,4-difluoropiperidine-1-carbonyl)-7-(4-(imidazo[1,2-a]pyridin-3-yl)-2,5-dioxo-2,5-dihydro-1H-pyrrol-3-yl)-1,2,3,4-tetrahydro-[1,4]diazepino[6,7,1-hi]indole-9-carbonitrile; 3-(2-(4,4-difluoropiperidine-1-carbonyl)-9-(trifluoromethyl)-1,2,3,4-tetrahydro-[1,4]diazepino[6,7,1-hi]indol-7-yl)-4-(imidazo[1,2-a]pyridin-3-yl)-1H-pyrrole-2,5-dione; 3-(2-(8-oxa-3-azabicyclo[3.2.1]octane-3-carbonyl)-9-(trifluoromethyl)-1,2,3,4-tetrahydro-[1,4]diazepino[6,7,1-hi]indol-7-yl)-4-(imidazo[1,2-a]pyridin-3-yl)-1H-pyrrole-2,5-dione; 3-(2-(4-(aminomethyl)piperidine-1-carbonyl)-9-fluoro-1,2,3,4-tetrahydro-[1,4]diazepino[6,7,1-hi]indol-7-yl)-4-(imidazo[1,2-a]pyridin-3-yl)-1H-pyrrole-2,5-dione; 3-(2-(4-(hydroxymethyl)piperidine-1-carbonyl)-9-(trifluoromethyl)-1,2,3,4-tetrahydro-[1,4]diazepino[6,7,1-hi]indol-7-yl)-4-(imidazo[1,2-a]pyridin-3-yl)-1H-pyrrole-2,5-dione; 2-(4-(hydroxymethyl)piperidine-1-carbonyl)-7-(4-(imidazo[1,2-a]pyridin-3-yl)-2,5-dioxo-2,5-dihydro-1H-pyrrol-3-yl)-1,2,3,4-tetrahydro-[1,4]diazepino[6,7,1-hi]indole-9-carbonitrile; 3-(9-fluoro-2-(3,3,4,4,5,5-hexafluoropiperidine-1-carbonyl)-1,2,3,4-tetrahydro-[1,4]diazepino[6,7,1-hi]indol-7-yl)-4-(imidazo[1,2-a]pyridin-3-yl)-1H-pyrrole-2,5-dione; 3-(9-fluoro-2-(3,3,5,5-tetrafluoropiperidine-1-carbonyl)-1,2,3,4-tetrahydro-[1,4]diazepino[6,7,1-hi]indol-7-yl)-4-(imidazo[1,2-a]pyridin-3-yl)-1H-pyrrole-2,5-dione; 3-(9-fluoro-2-(2,2,6,6-tetrafluoromorpholine-4-carbonyl)-1,2,3,4-tetrahydro-[1,4]diazepino[6,7,1-hi]indol-7-yl)-4-(imidazo[1,2-a]pyridin-3-yl)-1H-pyrrole-2,5-dione; 3-(2-(4,4-difluoro-3-hydroxypiperidine-1-carbonyl)-9-fluoro-1,2,3,4-tetrahydro-[1,4]diazepino[6,7,1-hi]indol-7-yl)-4-(imidazo[1,2-a]pyridin-3-yl)-1H-pyrrole-2,5-dione; 3-(2-(4-(difluoro(hydroxy)methyl)piperidine-1-carbonyl)-9-fluoro-1,2,3,4-tetrahydro-[1,4]diazepino[6,7,1-hi]indol-7-yl)-4-(imidazo[1,2-a]pyridin-3-yl)-1H-pyrrole-2,5-dione; 3-(2-(6,6-difluoro-1,4-oxazepane-4-carbonyl)-9-fluoro-1,2,3,4-tetrahydro-[1,4]diazepino[6,7,1-hi]indol-7-yl)-4-(imidazo[1,2-a]pyridin-3-yl)-1H-pyrrole-2,5-dione; 3-([1,2,4]triazolo[4,3-a]pyridin-3-yl)-4-(9-fluoro-2-(piperidine-1-carbonyl)-1,2,3,4-tetrahydro-[1,4]diazepino[6,7,1-hi]indol-7-yl)-1H-pyrrole-2,5-dione; 3-(9-fluoro-2-(piperidine-1-carbonyl-d10)-1,2,3,4-tetrahydro-[1,4]diazepino[6,7,1-hi]indol-7-yl)-4-(imidazo[1,2-a]pyridin-3-yl)-1H-pyrrole-2,5-dione; 3-(9-fluoro-2-(piperidine-1-carbonyl)-1,2,3,4-tetrahydro-[1,4]diazepino[6,7,1-hi]indol-7-yl-3,3,4,4-d4)-4-(imidazo[1,2-a]pyridin-3-yl)-1H-pyrrole-2,5-dione; 3-(9-fluoro-2-(4-(2,2,2-trifluoro-1-hydroxyethyl)piperidine-1-carbonyl)-1,2,3,4-tetrahydro-[1,4]diazepino[6,7,1-hi]indol-7-yl)-4-(imidazo[1,2-a]pyridin-3-yl)-1H-pyrrole-2,5-dione; 3-(9-fluoro-2-(4-((methylamino)methyl)piperidine-1-carbonyl)-1,2,3,4-tetrahydro-[1,4]diazepino[6,7,1-hi]indol-7-yl)-4-(imidazo[1,2-a]pyridin-3-yl)-1H-pyrrole-2,5-dione; 3-(2-(4-((dimethylamino)methyl)piperidine-1-carbonyl)-9-fluoro-1,2,3,4-tetrahydro-[1,4]diazepino[6,7,1-hi]indol-7-yl)-4-(imidazo[1,2-a]pyridin-3-yl)-1H-pyrrole-2,5-dione; 3-(2-(4-aminopiperidine-1-carbonyl)-9-fluoro-1,2,3,4-tetrahydro[1,4]diazepino[6,7,1-hi]indol-7-yl)-4-(imidazo[1,2-a]pyridin-3-yl)-1H-pyrrole-2,5-dione; 3-(9-fluoro-2-(4-(methylamino)piperidine-1-carbonyl)-1,2,3,4-tetrahydro-[1,4]diazepino[6,7,1-hi]indol-7-yl)-4-(imidazo[1,2-a]pyridin-3-yl)-1H-pyrrole-2,5-dione; 3-(2-(4-(dimethylamino)piperidine-1-carbonyl)-9-fluoro-1,2,3,4-tetrahydro-[1,4]diazepino[6,7,1-hi]indol-7-yl)-4-(imidazo[1,2-a]pyridin-3-yl)-1H-pyrrole-2,5-dione; 9-fluoro-7-(4-(imidazo[1,2-a]pyridin-3-yl)-2,5-dioxo-2,5-dihydro-1H-pyrrol-3-yl)-N-(piperidin-4-ylmethyl)-3,4-dihydro-[1,4]diazepino[6,7,1-hi]indole-2(1H)-carboxamide; 9-fluoro-7-(4-(imidazo[1,2-a]pyridin-3-yl)-2,5-dioxo-2,5-dihydro-1H-pyrrol-3-yl)-N-methyl-N-(piperidin-4-ylmethyl)-3,4-dihydro-[1,4]diazepino[6,7,1-hi]indole-2(1H)-carboxamide; 9-fluoro-7-(4-(imidazo[1,2-a]pyridin-3-yl)-2,5-dioxo-2,5-dihydro-1H-pyrrol-3-yl)-N-methyl-N-((1-methylpiperidin-4-yl)methyl)-3,4-dihydro-[1,4]diazepino[6,7,1-hi]indole-2(1H)-carboxamide; 3-(9-fluoro-2-((1R,4R)-5-methyl-2,5-diazabicyclo[2.2.1]heptane-2-carbonyl)-1,2,3,4-tetrahydro-[1,4]diazepino[6,7,1-hi]indol-7-yl)-4-(imidazo[1,2-a]pyridin-3-yl)-1H-pyrrole-2,5-dione; 3-(9-fluoro-2-(2-methyl-2,8-diazaspiro[4.5]decane-8-carbonyl)-1,2,3,4-tetrahydro-[1,4]diazepino[6,7,1-hi]indol-7-yl)-4-(imidazo[1,2-a]pyridin-3-yl)-1H-pyrrole-2,5-dione; 3-(9-fluoro-2-(8-methyl-2,8-diazaspiro[4.5]decane-2-carbonyl)-1,2,3,4-tetrahydro-[1,4]diazepino[6,7,1-hi]indol-7-yl)-4-(imidazo[1,2-a]pyridin-3-yl)-1H-pyrrole-2,5-dione; 3-(imidazo[1,2-a]pyridin-3-yl)-4-(2-(2,2,6,6-tetrafluoromorpholine-4-carbonyl)-9-(trifluoromethyl)-1,2,3,4-tetrahydro-[1,4]diazepino[6,7,1-hi]indol-7-yl)-1H-pyrrole-2,5-dione; 3-(2-(6,6-difluoro-1,4-oxazepane-4-carbonyl)-9-(trifluoromethyl)-1,2,3,4-tetrahydro-[1,4]diazepino[6,7,1-hi]indol-7-yl)-4-(imidazo[1,2-a]pyridin-3-yl)-1H-pyrrole-2,5-dione; 2-(4-(dimethylamino)piperidine-1-carbonyl)-7-(4-(imidazo[1,2-a]pyridin-3-yl)-2,5-dioxo-2,5-dihydro-1H-pyrrol-3-yl)-1,2,3,4-tetrahydro-[1,4]diazepino[6,7,1-hi]indole-9-carbonitrile; 9-cyano-7-(4-(imidazo[1,2-a]pyridin-3-yl)-2,5-dioxo-2,5-dihydro-1H-pyrrol-3-yl)-N-methyl-N-((1-methylpiperidin-4-yl)methyl)-3,4-dihydro-[1,4]diazepino[6,7,1-hi]indole-2(1H-carboxamide; 7-(4-(imidazo[1,2-a]pyridin-3-yl)-2,5-dioxo-2,5-dihydro-1H-pyrrol-3-yl)-2-(8-methyl-2,8-diazaspiro[4.5]decane-2-carbonyl)-1,2,3,4-tetrahydro-[1,4]diazepino[6,7,1-hi]indole-9-carbonitrile; 3-(8,9-difluoro-2-(piperidine-1-carbonyl)-1,2,3,4-tetrahydro-[1,4]diazepino[6,7,1-hi]indol-7-yl)-4-(imidazo[1,2-a]pyridin-3-yl)-1H-pyrrole-2,5-dione; or 3-(9-fluoro-2-(piperidine-1-carbonyl)-1,2,3,4-tetrahydro-[1,4]diazepino[6,7,1-hi]indol-7-yl)-4-(imidazo[1,2-a]pyridin-3-yl)-1H-pyrrole-2,5-dione (LY20900314).

In some embodiments, the substituted 3-Imidazo[1,2-a]pyridin-3-yl-4-(1,2,3,4-tetrahydro-[1,4]diazepino-[6,7,1-hi]indol-7-yl)pyrrole-2,5-dione is: 3-(imidazo[1,2-a]pyridin-3-yl)-4-(2-(piperidine-1-carbonyl)-9-(trifluoromethyl)-1,2,3,4-tetrahydro-[1,4]diazepino[6,7,1-hi]indol-7-yl)-1H-pyrrole-2,5-dione; 7-(4-(imidazo[1,2-a]pyridin-3-yl)-2,5-dioxo-2,5-dihydro-1H-pyrrol-3-yl)-2-(piperidine-1-carbonyl)-1,2,3,4-tetrahydro-[1,4]diazepino[6,7,1-hi]indole-9-carbonitrile; 3-(9-ethynyl-2-(piperidine-1-carbonyl)-1,2,3,4-tetrahydro-[1,4]diazepino[6,7,1-hi]indol-7-yl)-4-(imidazo[1,2-a]pyridin-3-yl)-1H-pyrrole-2,5-dione; 3-(9-fluoro-2-(4-(hydroxymethyl)piperidine-1-carbonyl)-1,2,3,4-tetrahydro-[1,4]diazepino[6,7,1-hi]indol-7-yl)-4-(imidazo[1,2-a]pyridin-3-yl)-1H-pyrrole-2,5-dione; 3-(2-(4,4-difluoropiperidine-1-carbonyl)-9-fluoro-1,2,3,4-tetrahydro-[1,4]diazepino[6,7,1-hi]indol-7-yl)-4-(imidazo[1,2-a]pyridin-3-yl)-1H-pyrrole-2,5-dione; 3-(2-(8-oxa-3-azabicyclo[3.2.1]octane-3-carbonyl)-9-fluoro-1,2,3,4-tetrahydro-[1,4]diazepino[6,7,1-hi]indol-7-yl)-4-(imidazo[1,2-a]pyridin-3-yl)-1H-pyrrole-2,5-dione; 3-(9-(difluoromethyl)-2-(piperidine-1-carbonyl)-1,2,3,4-tetrahydro-[1,4]diazepino[6,7,1-hi]indol-7-yl)-4-(imidazo[1,2-a]pyridin-3-yl)-1H-pyrrole-2,5-dione; 3-(2-(3,3-difluoropiperidine-1-carbonyl)-9-fluoro-1,2,3,4-tetrahydro-[1,4]diazepino[6,7,1-hi]indol-7-yl)-4-(imidazo[1,2-a]pyridin-3-yl)-1H-pyrrole-2,5-dione; 2-(4,4-difluoropiperidine-1-carbonyl)-7-(4-(imidazo[1,2-a]pyridin-3-yl)-2,5-dioxo-2,5-dihydro-1H-pyrrol-3-yl)-1,2,3,4-tetrahydro-[1,4]diazepino[6,7,1-hi]indole-9-carbonitrile; 3-(2-(8-oxa-3-azabicyclo[3.2.1]octane-3-carbonyl)-9-(trifluoromethyl)-1,2,3,4-tetrahydro-[1,4]diazepino[6,7,1-hi]indol-7-yl)-4-(imidazo[1,2-a]pyridin-3-yl)-1H-pyrrole-2,5-dione; 3-(2-(4-(hydroxymethyl)piperidine-1-carbonyl)-9-(trifluoromethyl)-1,2,3,4-tetrahydro-[1,4]diazepino[6,7,1-hi]indol-7-yl)-4-(imidazo[1,2-a]pyridin-3-yl)-1H-pyrrole-2,5-diene; 3-(9-fluoro-2-(3,3,4,4,5,5-hexafluoropiperidine-1-carbonyl)-1,2,3,4-tetrahydro-[1,4]diazepino[6,7,1-hi]indol-7-yl)-4-(imidazo[1,2-a]pyridin-3-yl)-1H-pyrrole-2,5-dione; 3-(9-fluoro-2-(3,3,5,5-tetrafluoropiperidine-1-carbonyl)-1,2,3,4-tetrahydro-[1,4]diazepino[6,7,1-hi]indol-7-yl)-4-(imidazo[1,2-a]pyridin-3-yl)-1H-pyrrole-2,5-dione; 3-(9-fluoro-2-(2,2,6,6-tetrafluoromorpholine-4-carbonyl)-1,2,3,4-tetrahydro-[1,4]diazepino[6,7,1-hi]indol-7-yl)-4-(imidazo[1,2-a]pyridin-3-yl)-1H-pyrrole-2,5-dione; 3-(2-(4,4-difluoro-3-hydroxypiperidine-1-carbonyl)-9-fluoro-1,2,3,4-tetrahydro-[1,4]diazepino[6,7,1-hi]indol-7-yl)-4-(imidazo[1,2-a]pyridin-3-yl)-1H-pyrrole-2,5-dione; 3-(2-(4-(difluoro(hydroxy)methyl)piperidine-1-carbonyl)-9-fluoro-1,2,3,4-tetrahydro-[1,4]diazepino[6,7,1-hi]indol-7-yl)-4-(imidazo[1,2-a]pyridin-3-yl)-1H-pyrrole-2,5-dione; 3-(2-(6,6-difluoro-1,4-oxazepane-4-carbonyl)-9-fluoro-1,2,3,4-tetrahydro-[1,4]diazepino[6,7,1-hi]indol-7-yl)-4-(imidazo[1,2-a]pyridin-3-yl)-1H-pyrrole-2,5-dione; 3-(9-fluoro-2-(piperidine-1-carbonyl-d10)-1,2,3,4-tetrahydro-[1,4]diazepino[6,7,1-hi]indol-7-yl)-4-(imidazo[1,2-a]pyridin-3-yl)-1H-pyrrole-2,5-dione; 3-(9-fluoro-2-(piperidine-1-carbonyl)-1,2,3,4-tetrahydro-[1,4]diazepino[6,7,1-hi]indol-7-yl)-3,3,4,4-d4)-4-(imidazo[1,2-a]pyridin-3-yl)-1H-pyrrole-2,5-dione; 3-(9-fluoro-2-(4-(2,2,2-trifluoro-1-hydroxyethyl)piperidine-1-carbonyl)-1,2,3,4-tetrahydro-[1,4]diazepino[6,7,1-hi]indol-7-yl)-4-(imidazo[1,2-a]pyridin-3-yl)-1H-pyrrole-2,5-dione; 3-(2-(4-((dimethylamino)methyl)piperidine-1-carbonyl)-9-fluoro-1,2,3,4-tetrahydro-[1,4]diazepino[6,7,1-hi]indol-7-yl)-4-(imidazo[1,2-a]pyridin-3-yl)-1H-pyrrole-2,5-dione; 3-(2-(4-(dimethylamino)piperidine-1-carbonyl)-9-fluoro-1,2,3,4-tetrahydro-[1,4]diazepino[6,7,1-hi]indol-7-yl)-4-(imidazol[1,2-a]pyridin-3-yl)-1H-pyrrole-2,5-dione; 9-fluoro-7-(4-(imidazo[1,2-a]pyridin-3-yl)-2,5-dioxo-2,5-dihydro-1H-pyrrol-3-yl)-N-methyl-N-((1-methylpiperidin4-yl)methyl)-3,4-dihydro-[1,4]diazepino[6,7,1-hi]indole-2(1H)-carboxamide; 3-(imidazo[1,2-a]pyridin-3-yl)-4-(2-(2,2,6,6-tetrafluoromorpholine-4-carbonyl)-9-(trifluoromethyl)-1,2,3,4-tetrahydro-[1,4]diazepino[6,7,1-hi]indol-7-yl)-1H-pyrrole-2,5-dione; 3-(2-(6,6-difluoro-1,4-oxazepane-4-carbonyl)-9-(trifluoromethyl)-1,2,3,4-tetrahydro-[1,4]diazepino[6,7,1-hi]indol-7-yl)-4-(imidazo[1,2-a]pyridin-3-yl)-1H-pyrrole-2,5-dione; 3-(8,9-difluoro-2-(piperidine-1-carbonyl)-1,2,3,4-tetrahydro-[1,4]diazepino[6,7,1-hi]indol-7-yl)-4-(imidazo[1,2-a]pyridin-3-yl)-1H-pyrrole-2,5-dione; or 3-(9-fluoro-2-(piperidine-1-carbonyl)-1,2,3,4-tetrahydro-[1,4]diazepino[6,7,1-hi]indol-7-yl)-4-(imidazo[1,2-a]pyridin-3-yl)-1H-pyrrole-2,5-dione. (LY2090314).

In some embodiments, the substituted 3-Imidazo[1,2-a]pyridin-3-yl-4-(1,2,3,4-tetrahydro-[1,4]diazepino-[6,7,1-hi]indol-7-yl)pyrrole-2,5-dione is 3-(9-fluoro-2-(piperidine-1-carbonyl)-1,2,3,4-tetrahydro-[1,4]diazepino[6,7,1-hi]indol-7-yl)-4-(imidazo[1,2-a]pyridin-3-yl)-1H-pyrrole-2,5-dione. (LY2090314).

The structures of the substituted 3-Imidazo[1,2-a]pyridin-3-yl-4-(1,2,3,4-tetrahydro-[1,4]diazepino-[6,7,1-hi]indol-7-yl)pyrrole-2,5-dione are shown below in Table 4.

TABLE 4

| | | |
|---|---|---|
| Compound I-1 | 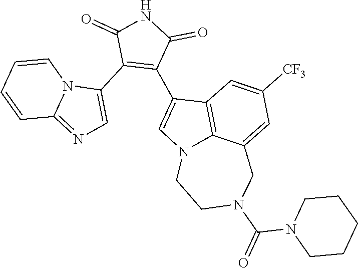 [SMILES: O=C1NC(=O)C(c2cnc3ccccn23)=C1c1cn2c3c(cc(C(F)(F)F)cc13)CN(C(=O)N1CCCCC1)CC2] | 3-(imidazo[1,2-a]pyridin-3-yl)-4-(2- (piperidine-1-carbonyl)-9-(trifluoromethyl)- 1,2,3,4-tetrahydro-[1,4]diazepino[6,7,1- hi]indol-7-yl)-1H-pyrrole-2,5-dione |
| Compound I-2 | 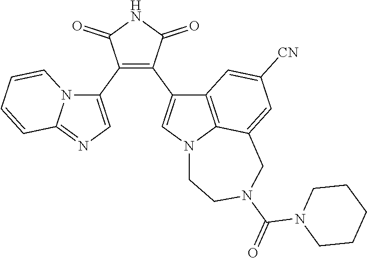 [SMILES: N#Cc1cc2c3c(c1)c(C1=C(c4cnc5ccccn45)C(=O)NC1=O)cn3CCN(C(=O)N1CCCCC1)C2] | 7-(4-(imidazo[1,2-a]pyridin-3-yl)-2,5-dioxo- 2,5-dihydro-1H-pyrrol-3-yl)-2-(piperidine-1- carbonyl)-1,2,3,4-tetrahydro- [1,4]diazepino[6,7,1-hi]indole-9-carbonitrile |
| Compound I-3 | 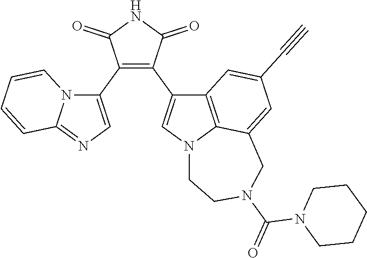 [SMILES: C#Cc1cc2c3c(c1)c(C1=C(c4cnc5ccccn45)C(=O)NC1=O)cn3CCN(C(=O)N1CCCCC1)C2] | 3-(9-ethynyl-2-(piperidine-1-carbonyl)-1,2,3,4- tetrahydro-[1,4]diazepino[6,7,1-hi]indol-7-yl)- 4-(imidazo[1,2-a]pyridin-3-yl)-1H-pyrrole-2,5- dione |
| Compound I-4 | 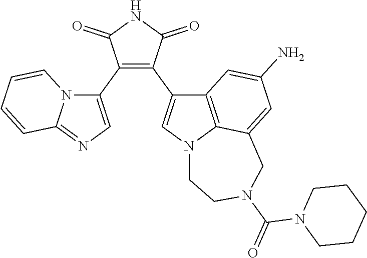 [SMILES: Nc1cc2c3c(c1)c(C1=C(c4cnc5ccccn45)C(=O)NC1=O)cn3CCN(C(=O)N1CCCCC1)C2] | 3-(9-amino-2-(piperidine-1-carbonyl)-1,2,3,4- tetrahydro-[1,4]diazepino[6,7,1-hi]indol-7-yl)- 4-(imidazo[1,2-a]pyridin-3-yl)-1H-pyrrole-2,5- dione |
| Compound I-5 | 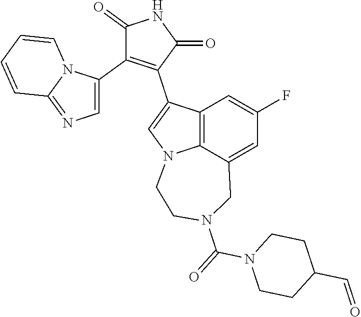 [SMILES: O=CC1CCN(C(=O)N2CCn3cc(C4=C(c5cnc6ccccn56)C(=O)NC4=O)c4cc(F)cc(c43)C2)CC1] | 1-(9-fluoro-7-(4-(imidazo[1,2-a]pyridin-3-yl)- 2,5-dioxo-2,5-dihydro-1H-pyrrol-3-yl)-1,2,3,4- tetrahydro-[1,4]diazepino[6,7,1-hi]indole-2- carbonyl)piperidine-4-carbaldehyde |
| Compound I-6 | 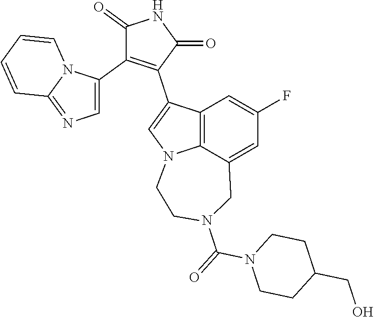 [SMILES: O=C1NC(=O)C(c2cnc3ccccn23)=C1c1cn2c3c(cc(F)cc13)CN(C(=O)N1CCC(CO)CC1)CC2] | 3-(9-fluoro-2-(4-(hydroxymethyl)piperidine-1- carbonyl)-1,2,3,4-tetrahydro- [1,4]diazepino[6,7,1-hi]indol-7-yl)-4- (imidazo[1,2-a]pyridin-3-yl)-1H-pyrrole-2,5- dione |
| Compound I-7 | 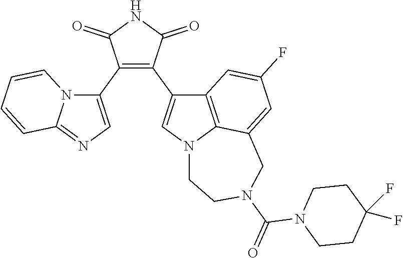 [SMILES: O=C1NC(=O)C(c2cnc3ccccn23)=C1c1cn2c3c(cc(F)cc13)CN(C(=O)N1CCC(F)(F)CC1)CC2] | 3-(2-(4,4-difluoropiperidine-1-carbonyl)-9- fluoro-1,2,3,4-tetrahydro- [1,4]diazepino[6,7,1-hi]indol-7-yl)-4- (imidazo[1,2-a]pyridin-3-yl)-1H-pyrrole-2,5- dione |
| Compound I-8 | 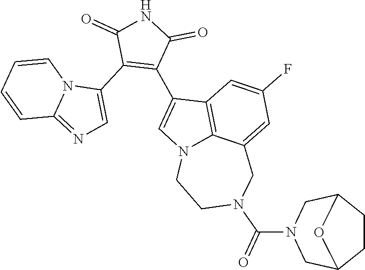 [SMILES: O=C1NC(=O)C(c2cnc3ccccn23)=C1c1cn2c3c(cc(F)cc13)CN(C(=O)N1CC3CCC(C1)O3)CC2] | 3-(2-(8-oxa-3-azabicyclo[3.2.1]octane-3- carbonyl)-9-fluoro-1,2,3,4-tetrahydro- [1,4]diazepino[6,7,1-hi]indol-7-yl)-4- (imidazo[1,2-a]pyridin-3-yl)-1H-pyrrole-2,5- dione |
| Compound I-9 | 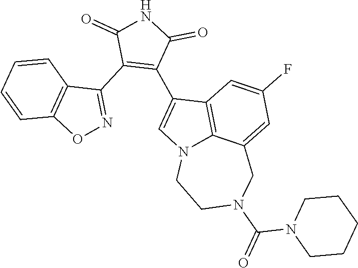 [SMILES: O=C1NC(=O)C(c2cn3c4c(cc(F)cc24)CN(C(=O)N2CCCCC2)CC3)=C1c1noc2ccccc12] | 3-(benzo[d]isoxazol-3-yl)-4-(9-fluoro-2- (piperidine-1-carbonyl)-1,2,3,4-tetrahydro- [1,4]diazepino[6,7,1-hi]indol-7-yl)-1H-pyrrole- 2,5-dione |
| Compound I-10 | 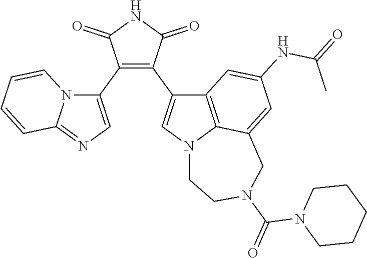 [SMILES: CC(=O)Nc1cc2c3c(c1)c(C1=C(c4cnc5ccccn45)C(=O)NC1=O)cn3CCN(C(=O)N1CCCCC1)C2] | N-(7-(4-(imidazo[1,2-a]pyridin-3-yl)-2,5-dioxo- 2,5-dihydro-1H-pyrrol-3-yl)-2-(piperidine-1- carbonyl)-1,2,3,4-tetrahydro- [1,4]diazepino[6,7,1-hi]indol-9-yl)acetamide |
| Compound I-11 | 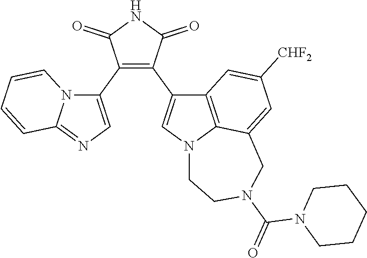 [SMILES: O=C1NC(=O)C(c2cnc3ccccn23)=C1c1cn2c3c(cc(C(F)F)cc13)CN(C(=O)N1CCCCC1)CC2] | 3-(9-(difluoromethyl)-2-(piperidine-1- carbonyl)-1,2,3,4-tetrahydro- [1,4]diazepino[6,7,1-hi]indol-7-yl)-4- (imidazo[1,2-a]pyridin-3-yl)-1H-pyrrole-2,5- dione |
| Compound I-12 | 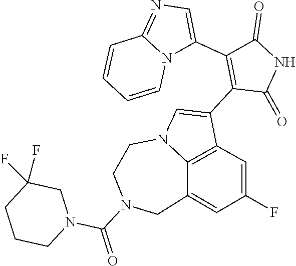 [SMILES: O=C1NC(=O)C(c2cnc3ccccn23)=C1c1cn2c3c(cc(F)cc13)CN(C(=O)N1CCCC(F)(F)C1)CC2] | 3-(2-(3,3-difluoropiperidine-1-carbonyl)-9- fluoro-1,2,3,4-tetrahydro- [1,4]diazepino[6,7,1-hi]indol-7-yl)-4- (imidazo[1,2-a]pyridin-3-yl)-1H-pyrrole-2,5- dione |
| Compound I-13 | 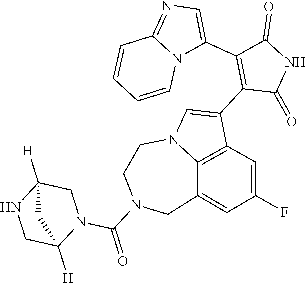 [SMILES: O=C1NC(=O)C(c2cnc3ccccn23)=C1c1cn2c3c(cc(F)cc13)CN(C(=O)N1C[C@H]3C[C@@H]1CN3)CC2] | 3-(2-((1R,4R)-2,5-diazabicyclo[2.2.1]heptane- 2-carbonyl)-9-fluoro-1,2,3,4-tetrahydro- [1,4]diazepino[6,7,1-hi]indol-7-yl)-4- (imidazo[1,2-a]pyridin-3-yl)-1H-pyrrole-2,5- dione |
| Compound I-14 | 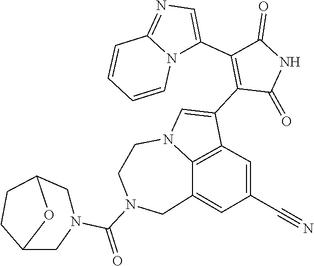 [SMILES: N#Cc1cc2c3c(c1)c(C1=C(c4cnc5ccccn45)C(=O)NC1=O)cn3CCN(C(=O)N1CC3CCC(C1)O3)C2] | 2-(8-oxa-3-azabicyclo[3.2.1]octane-3- carbonyl)-7-(4-(imidazo[1,2-a]pyridin-3-yl)- 2,5-dioxo-2,5-dihydro-1H-pyrrol-3-yl)-1,2,3,4- tetrahydro-[1,4]diazepino[6,7,1-hi]indole-9- carbonitrile |
| Compound I-15 | 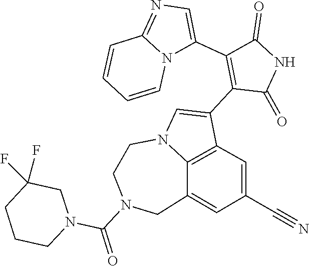 [SMILES: N#Cc1cc2c3c(c1)c(C1=C(c4cnc5ccccn45)C(=O)NC1=O)cn3CCN(C(=O)N1CCCC(F)(F)C1)C2] | 2-(3,3-difluoropiperidine-1-carbonyl)-7-(4- (imidazo[1,2-a]pyridin-3-yl)-2,5-dioxo-2,5- dihydro-1H-pyrrol-3-yl)-1,2,3,4-tetrahydro- [1,4]diazepino[6,7,1-hi]indole-9-carbonitrile |
| Compound I-16 | 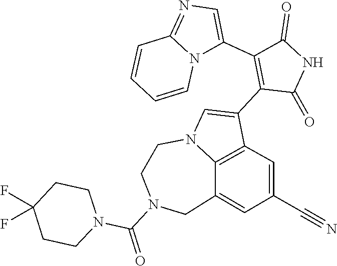 [SMILES: N#Cc1cc2c3c(c1)c(C1=C(c4cnc5ccccn45)C(=O)NC1=O)cn3CCN(C(=O)N1CCC(F)(F)CC1)C2] | 2-(4,4-difluoropiperidine-1-carbonyl)-7-(4- (imidazo[1,2-a]pyridin-3-yl)-2,5-dioxo-2,5- dihydro-1H-pyrrol-3-yl)-1,2,3,4-tetrahydro- [1,4]diazepino[6,7,1-hi]indole-9-carbonitrile |
| Compound I-17 | 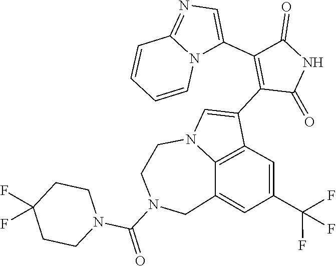 [SMILES: O=C1NC(=O)C(c2cnc3ccccn23)=C1c1cn2c3c(cc(C(F)(F)F)cc13)CN(C(=O)N1CCC(F)(F)CC1)CC2] | 3-(2-(4,4-difluoropiperidine-1-carbonyl)-9- (trifluoromethyl)-1,2,3,4-tetrahydro- [1,4]diazepino[6,7,1-hi]indol-7-yl)-4- (imidazo[1,2-a]pyridin-3-yl)-1H-pyrrole-2,5- dione |
| Compound I-18 | 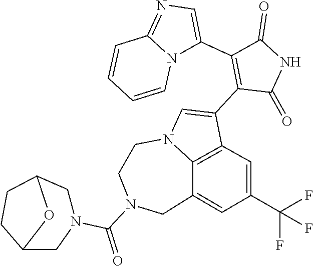 [SMILES: O=C1NC(=O)C(c2cnc3ccccn23)=C1c1cn2c3c(cc(C(F)(F)F)cc13)CN(C(=O)N1CC3CCC(C1)O3)CC2] | 3-(2-(8-oxa-3-azabicycio[3.2.1]octane-3- carbonyl)-9-(trifluoromethyl)-1,2,3,4- tetrahydro-[1,4]diazepino[6,7,1-hi]indol-7-yl)- 4-(imidazo[1,2-a]pyridin-3-yl)-1H-pyrrole-2,5- dione |
| Compound I-19 | 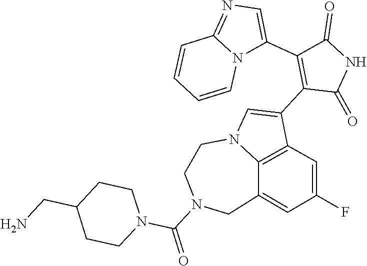 [SMILES: NCC1CCN(C(=O)N2CCn3cc(C4=C(c5cnc6ccccn56)C(=O)NC4=O)c4cc(F)cc(c43)C2)CC1] | 3-(2-(4-(aminomethyl)piperidine-1-carbonyl)- 9-fluoro-1,2,3,4-tetrahydro- [1,4]diazepino[6,7,1-hi]indol-7-yl)-4- (imidazo[1,2-a]pyridin-3-yl)-1H-pyrrole-2,5- dione |
| Compound I-20 | 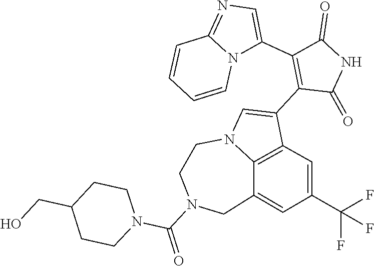 [SMILES: O=C1NC(=O)C(c2cnc3ccccn23)=C1c1cn2c3c(cc(C(F)(F)F)cc13)CN(C(=O)N1CCC(CO)CC1)CC2] | 3-(2-(4-(hydroxymethyl)piperidine-1- carbonyl)-9-(trifluoromethyl)-1,2,3.4- tetrahydro-[1,4]diazepino[6,7,1-hi]indol-7-yl)- 4-(imidazo[1,2-a]pyridin-3-yl)-1H-pyrrole-2,5- dione |
| Compound I-21 | 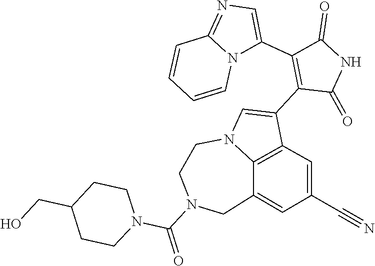 [SMILES: N#Cc1cc2c3c(c1)c(C1=C(c4cnc5ccccn45)C(=O)NC1=O)cn3CCN(C(=O)N1CCC(CO)CC1)C2] | 2-(4-(hydroxymethyl)piperidine-1-carbonyl)-7- (4-(imidazo[1,2-a]pyridin-3-yl)-2,5-dioxo-2,5- dihydro-1H-pyrrol-3-yl)-1,2,3,4-tetrahydro- [1,4]diazepino[6,7,1-hi]indole-9-carbonitrile |
| Compound I-22 | 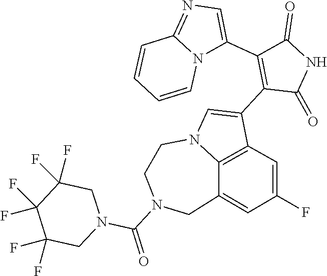 [SMILES: O=C1NC(=O)C(c2cnc3ccccn23)=C1c1cn2c3c(cc(F)cc13)CN(C(=O)N1CC(F)(F)C(F)(F)C(F)(F)C1)CC2] | 3-(9-fluoro-2-(3,3,4,4,5,5- hexafluoropiperidine-1-carbonyl)-1,2,3,4- tetrahydro-[1,4]diazepino[6,7,1-hi]indol-7-yl)- 4-(imidazo[1,2-a]pyridin-3-yl)-1H-pyrrole-2,5- dione |
| Compound I-23 | 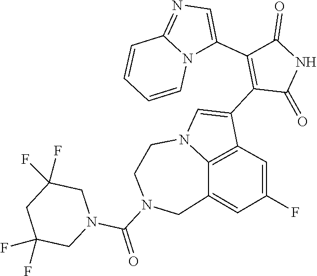 [SMILES: O=C1NC(=O)C(c2cnc3ccccn23)=C1c1cn2c3c(cc(F)cc13)CN(C(=O)N1CC(F)(F)CC(F)(F)C1)CC2] | 3-(9-fluoro-2-(3,3,5,5-tetrafluoropiperidine-1- carbonyl)-1,2,3,4-tetrahydro- [1,4]diazepino[6,7,1-hi]indol-7-yl)-4- (imidazo[1,2-a]pyridin-3-yl)-1H-pyrrole-2,5- dione |
| 1Compound I-24 | 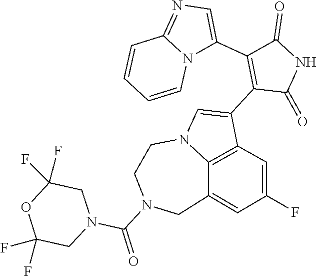 [SMILES: O=C1NC(=O)C(c2cnc3ccccn23)=C1c1cn2c3c(cc(F)cc13)CN(C(=O)N1CC(F)(F)OC(F)(F)C1)CC2] | 3-(9-fluoro-2-(2,2,6,6-tetrafluoromorpholine- 4-carbonyl)-1,2,3,4-tetrahydro- [1,4]diazepino[6,7,1-hi]indol-7-yl)-4- (imidazo[1,2-a]pyridin-3-yl)-1H-pyrrole-2,5- dione |
| Compound I-25 | 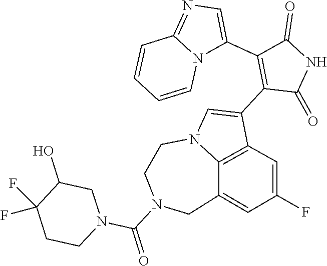 [SMILES: O=C1NC(=O)C(c2cnc3ccccn23)=C1c1cn2c3c(cc(F)cc13)CN(C(=O)N1CCC(F)(F)C(O)C1)CC2] | 3-(2-(4,4-difluoro-3-hydroxypiperidine-1- carbonyl)-9-fluoro-1,2,3,4-tetrahydro- [1,4]diazepino[6,7,1-hi]indol-7-yl)-4- (imidazo[1,2-a]pyridin-3-yl)-1H-pyrrole-2,5- dione |
| Compound I-26 | 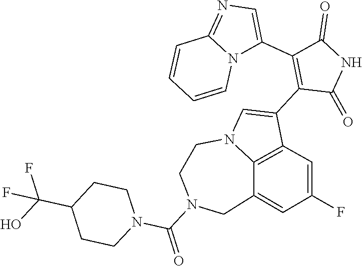 [SMILES: O=C1NC(=O)C(c2cnc3ccccn23)=C1c1cn2c3c(cc(F)cc13)CN(C(=O)N1CCC(C(O)(F)F)CC1)CC2] | 3-(2-(4-(difluoro(hydroxy)methyl)piperidine-1- carbonyl)-9-fluoro-1,2,3,4-tetrahydro- [1,4]diazepino[6,7,1-hi]indol-7-yl)-4- (imidazo[1,2-a]pyridin-3-yl)-1H-pyrrole-2,5- dione |
| Compound I-27 | 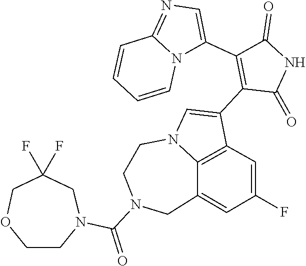 [SMILES: O=C1NC(=O)C(c2cnc3ccccn23)=C1c1cn2c3c(cc(F)cc13)CN(C(=O)N1CCOCC(F)(F)C1)CC2] | 3-(2-(6,6-difluoro-1,4-oxazepane-4-carbonyl)- 9-fluoro-1,2,3,4-tetrahydro- [1,4]diazepino[6,7,1-hi]indol-7-yl)-4- (imidazo[1,2-a]pyridin-3-yl)-1H-pyrrole-2,5- dione |
| Compound I-28 | 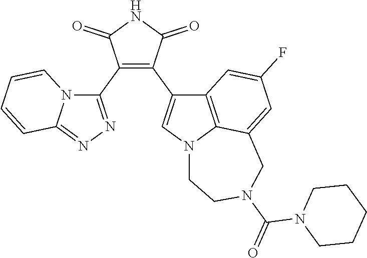 [SMILES: O=C1NC(=O)C(c2nnc3ccccn23)=C1c1cn2c3c(cc(F)cc13)CN(C(=O)N1CCCCC1)CC2] | 3-([1,2,4]triazolo[4,3-a]pyridin-3-yl)-4-(9- fluoro-2-(piperidine-1-carbonyl)-1,2,3,4- tetrahydro-[1,4]diazepino[6,7,1-hi]indol-7-yl)- 1H-pyrrole-2,5-dione |
| Compound I-29 | 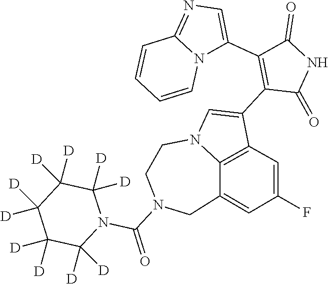 [SMILES: [2H]C1([2H])N(C(=O)N2CCn3cc(C4=C(c5cnc6ccccn56)C(=O)NC4=O)c4cc(F)cc(c43)C2)C([2H])([2H])C([2H])([2H])C([2H])([2H])C1([2H])[2H]] | 3-(9-fluoro-2-(piperidine-1-carbonyl-d10)- 1,2,3,4-tetrahydro-[1,4]diazepino[6,7,1- hi]indol-7-yl)-4-(imidazo[1,2-a]pyridin-3-yl)- 1H-pyrrole-2,5-dione |
| Compound I-30 | 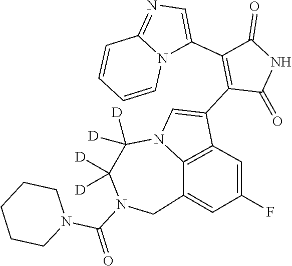 [SMILES: [2H]C1([2H])N(C(=O)N2CCCCC2)Cc2cc(F)cc3c(C4=C(c5cnc6ccccn56)C(=O)NC4=O)cn(c23)C1([2H])[2H]] | 3-(9-fluoro-2-(piperidine-1-carbonyl)-1,2,3,4- tetrahydro-[1,4]diazepino[6,7,1-hi]indol-7-yl- 3,3,4,4-d4)-4-(imidazo[1,2-a]pyridin-3-yl)-1H- pyrrole-2,5-dione |
| Compound I-31 | 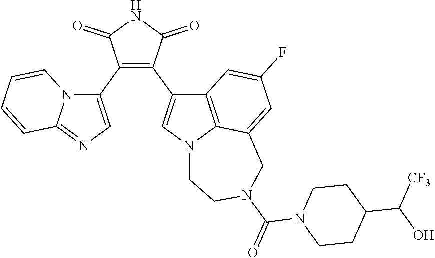 [SMILES: O=C1NC(=O)C(c2cnc3ccccn23)=C1c1cn2c3c(cc(F)cc13)CN(C(=O)N1CCC(C(O)C(F)(F)F)CC1)CC2] | 3-(9-fluoro-2-(4-(2,2,2-trifluoro-1- hydroxyethyl)piperidine-1-carbonyl)-1,2,3,4- tetrahydro-[1,4]diazepino[6,7,1-hi]indol-7-yl)- 4-(imidazo[1,2-a]pyridin-3-yl)-1H-pyrrole-2,5- dione |
| Compound I-32 | 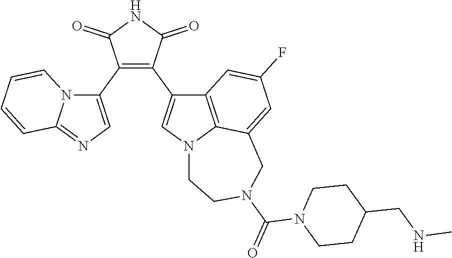 [SMILES: CNCC1CCN(C(=O)N2CCn3cc(C4=C(c5cnc6ccccn56)C(=O)NC4=O)c4cc(F)cc(c43)C2)CC1] | 3-(9-fluoro-2-(4- ((methylamino)methyl)piperidine-1-carbonyl)- 1,2,3,4-tetrahydro-[1,4]diazepino[6,7,1- hi]indol-7-yl)-4-(imidazo[1,2-a]pyridin-3-yl)- 1H-pyrrole-2,5-dione |
| Compound I-33 | 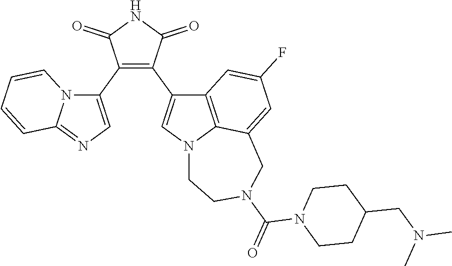 [SMILES: CN(C)CC1CCN(C(=O)N2CCn3cc(C4=C(c5cnc6ccccn56)C(=O)NC4=O)c4cc(F)cc(c43)C2)CC1] | 3-(2-(4-((dimethylamino)methyl)piperidine-1- carbonyl)-9-fluoro-1,2,3,4-tetrahydro- [1,4]diazepino[6,7,1-hi]indol-7-yl)-4- (imidazo[1,2-a]pyridin-3-yl)-1H-pyrrole-2,5- dione |
| Compound I-34 | 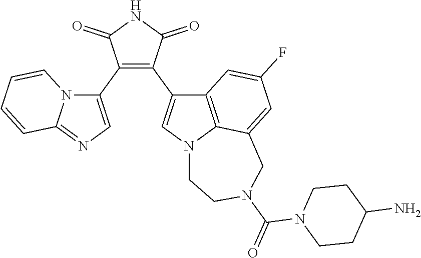 [SMILES: NC1CCN(C(=O)N2CCn3cc(C4=C(c5cnc6ccccn56)C(=O)NC4=O)c4cc(F)cc(c43)C2)CC1] | 3-(2-(4-aminopiperidine-1-carbonyl)-9-fluoro- 1,2,3,4-tetrahydro-[1,4]diazepino[6,7,1- hi]indol-7-yl)-4-(imidazo[1,2-a]pyridin-3-yl)- 1H-pyrrole-2,5-dione |
| Compound I-35 | 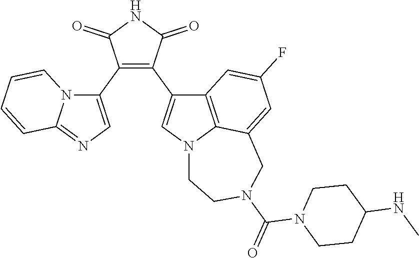 [SMILES: CNC1CCN(C(=O)N2CCn3cc(C4=C(c5cnc6ccccn56)C(=O)NC4=O)c4cc(F)cc(c43)C2)CC1] | 3-(9-fluoro-2-(4-(methylamino)piperidine-1- carbonyl)-1,2,3,4-tetrahydro- [1,4]diazepino[6,7,1-hi]indol-7-yl)-4- [imidazo[1,2-a]pyridin-3-yl)-1H-pyrrole-2,5- dione |
| Compound I-36 | 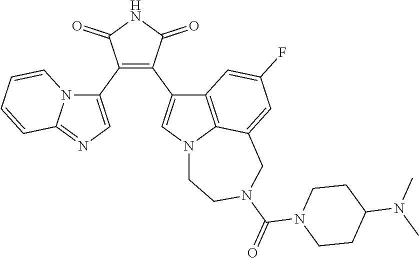 [SMILES: CN(C)C1CCN(C(=O)N2CCn3cc(C4=C(c5cnc6ccccn56)C(=O)NC4=O)c4cc(F)cc(c43)C2)CC1] | 3-(2-(4-(dimethylamino)piperidine-1- carbonyl)-9-fluoro-1,2,3,4-tetrahydro- [1,4]diazepino[6,7,1-hi]indol-7-yl)-4- (imidazo[1,2-a]pyridin-3-yl)-1H-pyrrole-2,5- dione |
| Compound I-37 | 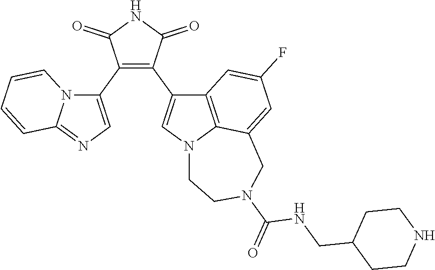 [SMILES: O=C1NC(=O)C(c2cnc3ccccn23)=C1c1cn2c3c(cc(F)cc13)CN(C(=O)NCC1CCNCC1)CC2] | 9-fluoro-7-(4-(imidazo[1,2-a]pyridin-3-yl)-2,5- dioxo-2,5-dihydro-1H-pyrrol-3-yl)-N- (piperidin-4-ylmethyl)-3,4-dihydro- [1,4]diazepino[6,7,1-hi]indole-2(1H)- carboxamide |
| Compound I-38 | 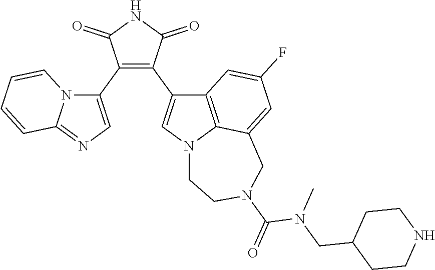 [SMILES: CN(CC1CCNCC1)C(=O)N1CCn2cc(C3=C(c4cnc5ccccn45)C(=O)NC3=O)c3cc(F)cc(c32)C1] | 9-fluoro-7-(4-(imidazo[1,2-a]pyridin-3-yl)-2,5- dioxo-2,5-dihydro-1H-pyrrol-3-yl)-N-methyl- N-(piperidin-4-ylmethyl)-3,4-dihydro- [1,4]diazepino[6,7,1-hi]indole-2(1H)- carboxamide |
| Compound I-39 | 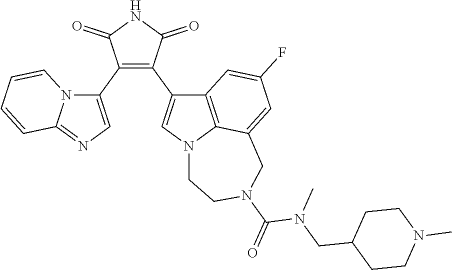 [SMILES: CN1CCC(CN(C)C(=O)N2CCn3cc(C4=C(c5cnc6ccccn56)C(=O)NC4=O)c4cc(F)cc(c43)C2)CC1] | 9-fluoro-7-(4-(imidazo[1,2-a]pyridin-3-yl)-2,5- dioxo-2,5-dihydro-1H-pyrrol-3-yl)-N-methyl- N-((1-methylpiperidin-4-yl)methyl)-3,4- dihydro-[1,4]diazepino[6,7,1-hi]indole-2(1H)- carboxamide |
| Compound I-40 | 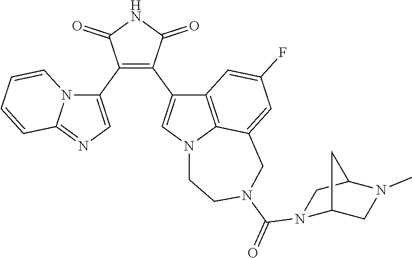 [SMILES: CN1CC2CC1CN2C(=O)N1CCn2cc(C3=C(c4cnc5ccccn45)C(=O)NC3=O)c3cc(F)cc(c32)C1] | 3-(9-fluoro-2-((1R,4R)-5-methyl-2,5- diazabicyclo[2.2.1]heptane-2-carbonyl)- 1,2,3,4-tetrahydro-[1,4]diazepino[6,7,1- hi]indol-7-yl)-4-(imidazo[1,2-a]pyridin-3-yl)- 1H-pyrrole-2,5-dione |
| Compound I-41 | 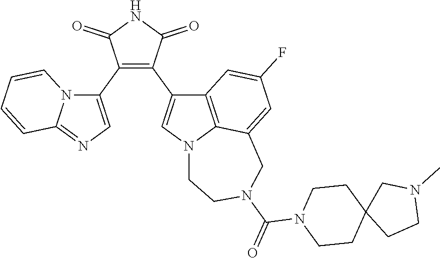 [SMILES: CN1CCC2(CCN(C(=O)N3CCn4cc(C5=C(c6cnc7ccccn67)C(=O)NC5=O)c5cc(F)cc(c54)C3)CC2)C1] | 3-(9-fluoro-2-(2-methyl-2,8- diazaspiro[4.5]decane-8-carbonyl)-1,2,3,4- tetrahydro-[1,4]diazepino[6,7,1-hi]indol-7-yl)- 4-(imidazo[1,2-a]pyridin-3-yl)-1H-pyrrole-2,5- dione |
| Compound I-42 | 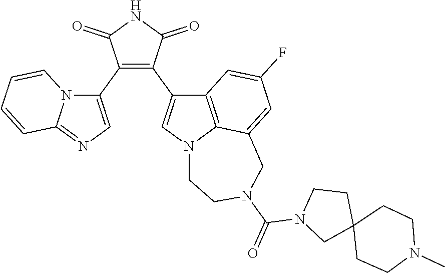 [SMILES: CN1CCC2(CC1)CCN(C(=O)N1CCn3cc(C4=C(c5cnc6ccccn56)C(=O)NC4=O)c4cc(F)cc(c43)C1)C2] | 3-(9-fluoro-2-(8-methyl-2,8- diazaspiro[4.5]decane-2-carbonyl)-1,2,3,4- tetrahydro-[1,4]diazepino[6,7,1-hi]indol-7-yl)- 4-(imidazo[1,2-a]pyridin-3-yl)-1H-pyrrole-2,5- dione |
| Compound I-43 | 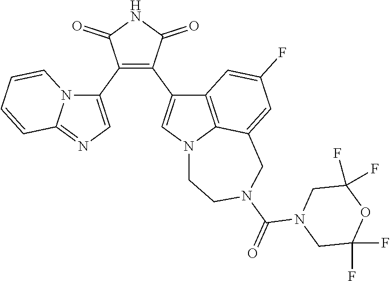 [SMILES: O=C1NC(=O)C(c2cnc3ccccn23)=C1c1cn2c3c(cc(F)cc13)CN(C(=O)N1CC(F)(F)OC(F)(F)C1)CC2] | 3-(imidazo[1,2-a]pyridin-3-yl)-4-(2-(2,2,6,6- tetrafluoromorpholine-4-carbonyl)-9- (trifluoromethyl)-1,2,3,4-tetrahydro- [1,4]diazepino[6,7,1-hi]indol-7-yl)-1H-pyrrole- 2,5-dione |
| Compound I-44 | 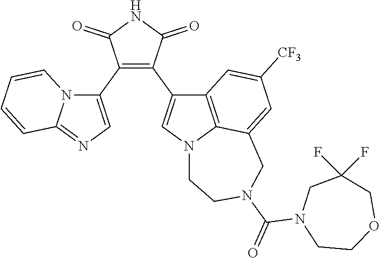 [SMILES: O=C1NC(=O)C(c2cnc3ccccn23)=C1c1cn2c3c(cc(C(F)(F)F)cc13)CN(C(=O)N1CCOCC(F)(F)C1)CC2] | 3-(2-(6,6-difluoro-1,4-oxazepane-4-carbonyl)- 9-(trifluoromethyl)-1,2,3,4-tetrahydro- [1,4]diazepino[6,7,1-hi]indol-7-yl)-4- (imidazo[1,2-a]pyridin-3-yl)-1H-pyrrole-2,5- dione |
| Compound I-45 | 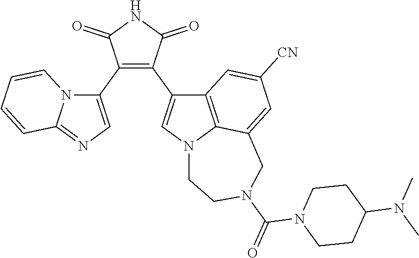 [SMILES: CN(C)C1CCN(C(=O)N2CCn3cc(C4=C(c5cnc6ccccn56)C(=O)NC4=O)c4cc(C#N)cc(c43)C2)CC1] | 2-(4-(dimethylamino)piperidine-1-carbonyl)-7- (4-(imidazo[1,2-a]pyridin-3-yl)-2,5-dioxo-2,5- dihydro-1H-pyrrol-3-yl)-1,2,3,4-tetrahydro- [1,4]diazepino[6,7,1-hi]indoie-9-carbonitrile |
| Compound I-46 | 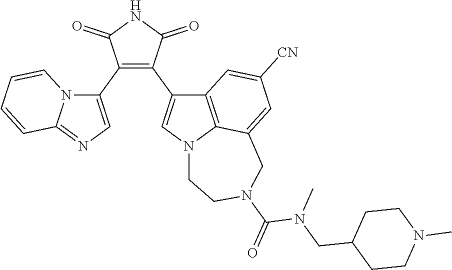 [SMILES: CN1CCC(CN(C)C(=O)N2CCn3cc(C4=C(c5cnc6ccccn56)C(=O)NC4=O)c4cc(C#N)cc(c43)C2)CC1] | 9-cyano-7-(4-(imidazo[1,2-a]pyridin-3-yl)-2,5- dioxo-2,5-dihydro-1H-pyrrol-3-yl)-N-methyl- N-((1-methylpiperidin-4-yl)methyl)-3,4- dihydro-[1,4]diazepino[6,7,1-hi]indole-2(1H)- carboxamide |
| Compound I-47 | 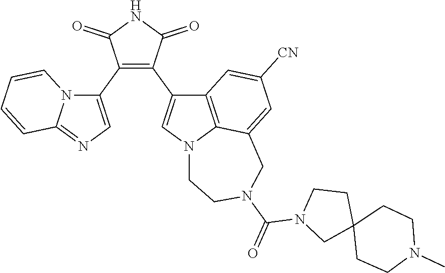 [SMILES: CN1CCC2(CC1)CCN(C(=O)N1CCn3cc(C4=C(c5cnc6ccccn56)C(=O)NC4=O)c4cc(C#N)cc(c43)C1)C2] | 7-(4-(imidazo[1,2-a]pyridin-3-yl)-2,5-dioxo- 2,5-dihydro-1H-pyrrol-3-yl)-2-(8-methyl-2,8- diazaspiro[4.5]decane-2-carbonyl)-1,2,3,4- tetrahydro-[1,4]diazepino[6,7,1-hi]indole-9- carbonitrile |
| Compound I-48 | 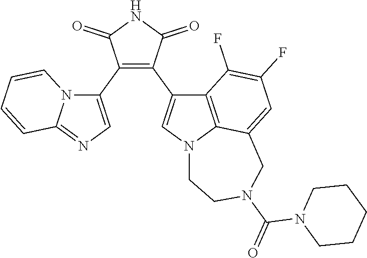 [SMILES: O=C1NC(=O)C(c2cnc3ccccn23)=C1c1cn2c3c(cc(F)c(F)c13)CN(C(=O)N1CCCCC1)CC2] | 3-(8,9-difluoro-2-(piperidine-1-carbonyl)- 1,2,3,4-tetrahydro-[1,4]diazepino[6,7,1- hi]indol-7-yl)-4-(imidazo[1,2-a]pyridin-3-yl)- 1H-pyrrole-2,5-dione |

In other embodiments, Wnt agonist and/or GSK3 inhibitor as described in WO 2018/125746, US 20180214458 and U.S. Ser. No. 62/608,663 the contents of which are each incorporated by reference in their entireties.

### HDAC Inhibitors

Histone deacetylases (HDAC) are a class of enzymes that remove acetyl groups (O═C—CH3) from an ε-N-acetyl lysine amino acid on a histone, allowing the histones to wrap the DNA more tightly. This is important because DNA is wrapped around histones, and DNA expression is regulated by acetylation and de-acetylation.

HDACs are classified in four classes depending on sequence homology to the yeast original enzymes and domain organization. The HDAC classes include HDACI, HDAC IIA HDAC IIB, HDAC III and HDAC IV.

Histone deacetylase (HDAC) inhibitors (HDACi, HDIs) are chemical compounds that inhibit histone deacetylases.

Thus, “HDAC inhibitor” refers to an agent capable of the decreasing the expression or enzymatic activity of HDAC. For example HDAC inhibitor results in a decrease in histone deacetylation of a target gene in a cell.

In certain embodiments, the HDAC inhibitor decreases the expression or enzymatic activity of HDAC by at least 5, 10, 20, 30, 40, 50, 60, 70, 80, 90, or 100% relative to a control, for example relative to a baseline level of activity.

In certain embodiments, the HDAC inhibitor decreases histone deacetylation of a target gene by at least 5, 10, 20, 30, 40, 50, 60, 70, 80, 90, or 100% relative to a control, for example relative to a baseline level of activity.

In some embodiments, the HDAC inhibitor increases expression or activity of a target gene by at least 5, 10, 20, 30, 40, 50, 60, 70, 80, 90, or 100% relative to a control, for example relative to a baseline level of activity.

In some embodiments, the HDAC inhibitor decreases expression or enzymatic activity of HDAC by at least about 1.1, 1.2, 1.3, 1.4, 1.5, 1.6, 1.7, 1.8, 1.9, 2, 3, 4, 5, 6, 7, 8, 9, 10, 15, 20, 30, 40, 50, 60, 70, 80, 90, 100, 200, 500, 1000-fold or more relative to a control, for example relative to a baseline level of activity.

In some embodiments, the HDAC inhibitor decreases histone deacetylation of a target gene by at least about 1.1, 1.2, 1.3, 1.4, 1.5, 1.6, 1.7, 1.8, 1.9, 2, 3, 4, 5, 6, 7, 8, 9, 10, 15, 20, 30, 40, 50, 60, 70, 80, 90, 100, 200, 500, 1000-fold or more relative to a control, for example relative to a baseline level of activity.

In some embodiments, the HDAC inhibitor increases expression or activity of a target gene by at least about 1.1, 1.2, 1.3, 1.4, 1.5, 1.6, 1.7, 1.8, 1.9, 2, 3, 4, 5, 6, 7, 8, 9, 10, 15, 20, 30, 40, 50, 60, 70, 80, 90, 100, 200, 500, 1000-fold or more relative to a control, for example relative to a baseline level of activity.

TABLE 5

| | | Chemo- | Mechanism | Class | HDAC | | Lgr5+ | Perilymph | Formulation |
|---|---|---|---|---|---|---|---|---|---|
| Agent | CAS | type | HDAC Inhib | selectivity | Potene | | Assay | Conc | Conc |
| Sodium | 1069-66-5 | Acid | 1, 2, 3, 8 | Class I | 39-161 | μM | 100 μM- | 100 μM- | 100 mM- |
| Valproate | | | | | | | 4 mM | 4 mM | 4000 mM |
| 2-hexyl-4- | 96017-59-3 | Acid | 1, 2, 3, 8 | Class I | 13 | μM | 100 μM- | 100 μM- | 100 mM- |
| pentynoic acid | | | | | | | 4 mM | 4 mM | 4000 mM |
| Na | 1716-12-7 | Acid | 1, 2, 3, 8 | Class I > | 9-16 | μM | 100 μM- | 100 μM- | 100 mM- |
| phenylbutyrate | | | | Class IIb | | | 4 mM | 4 mM | 4000 mM |

In various embodiments, the methods and compositions of the invention include use an HDAC inhibitor. Exemplary HDAC inhibitors are provided in Table 6

TABLE 6

| Class | Agent | CAS |
|---|---|---|
| Aliphatic Acid | Butyrate | 107-92-6 |
| Aliphatic Acid | Phenyl butyrate | 1821-12-1 |
| Aliphatic Acid | Valproic Acid | 99-66-1 |
| Aliphatic Acid Ester | AN-9 | 122110-53-6 |
| Amine | 932718-22-4 | 932718-22-4 |
| Benzamide | 4SC-202 | 1186222-89-8 |
| Benzamide | BML-210 | 537034-17-6 |
| Benzamide | Chidamide | 743438-44-0 |
| Benzamide | Entinostat (MS-275) | 209783-80-2 |
| Benzamide | HDAC Inhibitor IV | 537034-15-4 |
| Benzamide | Mocetinostat (MGCD0103) | 726169-73-9 |
| Benzamide | NKL 22 | 537034-15-4 |
| Benzamide | RGFP109 | 1215493-56-3 |
| Benzamide | RGFP136 | 1215493-97-2 |
| Benzamide | RGFP966 | 1357389-11-7 |
| Benzamide | Tacedinaline | 112522-64-2 |
| Benzamide | TC-H 106, HDAC Inhibitor VII | 937039-45-7 |
| Cyclic peptide | Apicidin | 183506-66-3 |
| Cyclic peptide | Dihydrochlamydocin | 52574-64-8 |
| Cyclic peptide | HC Toxin | 83209-65-8 |
| Cyclic peptide | Romidepsin | 128517-07-7 |
| Cyclic Peptide | Thailandepsin A | 1269219-30-8 |
| Cyclic peptide | Trapoxin A | 133155-89-2 |
| Epoxide | (−)-Depudecin | 139508-73-9 |
| Epoxide | Parthenolide | 20554-84-1 |
| Hydroxamate | (S)-HDAC-42 | 935881-37-1 |
| Hydroxamate | 4-(dimethylamino)-N-[6- | 193551-00-7 |
| | (hydroxyamino)-6-oxohexyl]- | |
| | benzamide | |
| Hydroxamate | 4-iodo-SAHA | 1219807-87-0 |
| Hydroxamate | 4SC-201 (Resminostat) | 864814-88-0 |
| Hydroxamate | ACY1215 | 1316214-52-4 |
| Hydroxamate | APHA Compound 8 | 676599-90-9 |
| Hydroxamate | BRD9757 | 1423058-85-8 |
| Hydroxamate | Bufexamac | 2438-72-4 |
| Hydroxamate | Butyrylhydroxamic acid | 4312-91-8 |
| Hydroxamate | CAY10603 | 1045792-66-2 |
| Hydroxamate | CBHA | 174664-65-4 |
| Hydroxamate | CG200745 | 936221-33-9 |
| Hydroxamate | CHR-3996 | 1256448-47-1 |
| Hydroxamate | CUDC-101 | 1012054-59-9 |
| Hydroxamate | Droxinostat | 99873-43-5 |
| Hydroxamate | HDAC Inhibitor II | 174664-65-4 |
| Hydroxamate | HDAC Inhibitor VI | 926908-04-5 |
| Hydroxamate | HDAC Inhibitor XXIV | 854779-95-6 |
| Hydroxamate | HDAC6 Inhibitor III | 1450618-49-1 |
| Hydroxamate | HDAC-IN-1 | 1239610-44-6 |
| Hydroxamate | HNHA | 926908-04-5 |
| Hydroxamate | HPOB | 1429651-50-2 |
| Hydroxamate | ITF2357 | 497833-27-9 |
| Hydroxamate | ITF2357 (Givinostat) | 497833-27-9 |
| Hydroxamate | LAQ-824 | 591207-53-3 |
| Hydroxamate | LBH-589 (panobinostat) | 404950-80-7 |
| Hydroxamate | LMK235 | 1418033-25-6 |
| Hydroxamate | M 344 | 251456-60-7 |
| Hydroxamate | MC 1568 | 852475-26-4 |
| Hydroxamate | Nexturastat A | 1403783-31-2 |
| Hydroxamate | NSC 57457 | 6953-61-3 |
| Hydroxamate | Oxamflatin | 151720-43-3 |
| Hydroxamate | PCI-24781 (Abexinostat) | 783355-60-2 |
| Hydroxamate | PCI-34051 | 950762-95-5 |
| Hydroxamate | PDX-101 (belinostat) | 866323-14-0 |
| Hydroxamate | Pyroxamide | 382180-17-8 |
| Hydroxamate | SAHA (Zolinza, vorinostat) | 149647-78-9 |
| Hydroxamate | SB939 (Pracinostat) | 929016-96-6 |
| Hydroxamate | SBHA | 38937-66-5 |
| Hydroxamate | Scriptaid | 287383-59-9 |
| Hydroxamate | Tefinostat (CHR-2845) | 914382-60-8 |
| Hydroxamate | Trichostatin A (TSA) | 58880-19-6 |
| Hydroxamate | Tubacin | 537049-40-4 |
| Hydroxamate | Tubastatin A | 1252003-15-8 |
| Hydroxamate | VAHA | 106132-78-9 |
| Ketone | Compound 43 | 891259-76-0 |
| Ketone - a-ketoamides | 436150-82-2 | 436150-82-2 |
| Ketone - CF3 | Compound 27 | 946499-86-1 |
| Ketone - CF3 | Compound 6e | 946500-31-8 |
| Ketone - CF3 | Compound 6H | 946500-39-6 |
| Non classical | Tasquinimod | 254964-60-8 |
| Non classical | TMP269 | 1314890-29-3 |
| Polyketide | Ratjadone A | 163564-92-9 |
| Silylalcohol | 1587636-32-5 | 1587636-32-5 |
| Sulphonamide | 1587636-33-6 | 1587636-33-6 |
| Sulphonamide | 329967-25-1 | 329967-25-1 |
| Sulphonyl Urea | 960130-17-0 | 960130-17-0 |
| Thioester | HDAC Inhibitor XXII | 848354-66-5 |
| Thioester | KD 5170 | 940943-37-3 |
| Thioester | PTACH | 848354-66-5 |
| Thioester | TCS HDAC6 20b | 956154-63-5 |
| Thioketone | SIRT1/2 Inhibitor VII | 143034-06-4 |
| Thiol | 1368806-68-1 | 1368806-68-1 |
| Thiol | 1428536-05-3 | 1428536-05-3 |
| Thiol | 827036-76-0 | 827036-76-0 |
| Thiol | 828920-13-4 | 828920-13-4 |
| Thiol | 908860-21-9 | 908860-21-9 |
| Tropones | 1411673-95-4 | 1411673-95-4 |
| Tropones | 46189-88-2 | 46189-88-2 |

In some embodiments the HDAC inhibitor is a class I HDAC inhibitor. In these embodiments, the class I HDAC inhibitor is a short chain carboxylic acid. In one embodiment, the HDAC inhibitor is valproic acid (VPA), 2-hexyl-4-pentynoic acid, or Na phenylbutyrate. In some embodiments, the HDAC inhibitor is valproic acid (VPA).

As used herein the terms “valproic acid”, “VPA” and “sodium valproate” are used interchangeably to refer to the same compound.

### Measurement of Sensorineural Hearing Loss

Hearing loss can be assessed by several different tests. Such tests may determine the audibility of a sound to a patient and/or the intelligibility of the sound to a patient prior to or after treatment. The audibility of a sound is a measure of a patient's ability to detect the sound (i.e. whether the patient can determine the presence or absence of a sound). The intelligibility of a sound is a measure of a patient's ability to correctly identify the sound. For instance, hearing is assessed according to whether a patient can correctly identify a word or not. A patient with hearing loss may therefore neither be able to detect a sound nor correctly identify it (i.e. the sound is inaudible and unintelligible). However, audibility is not necessarily associated with intelligibility, and a patient may, for example, be able to detect a sound, but not correctly identify it (i.e. the sound is audible but unintelligible).

Pure Tone Audiometry

Assessment of a patient's audibility function is typically carried out by an audiologist using an audiometer in a hearing test known as pure tone audiometry. Pure tone audiometry is a standard test used to assess the audibility of a sounds and is described in detail elsewhere (see, for example, Katz, J., Medwetsky, L., Burkard, R., & Hood, L. (2009) Handbook of Clinical Audiology. Philadelphia, Pa.: Lippincott Williams and Wilkins). Pure tone audiometry is typically carried out in a sound-treated booth, which reduces ambient noise levels that may interfere with the detection of low-level sound stimuli.

In pure tone audiometry, a patient is exposed to pure tone stimuli at specific frequencies to determine the patient's hearing threshold at each frequency. Standard audiometry measures a patient's pure tone hearing threshold at each of the following frequencies 0.25 kHz, 0.5 kHz, 1 kHz, 2 kHz, 3 kHz, 4 kHz, 6 kHz and 8 kHz. However, a patient's hearing threshold does not need to be determined at all of these frequencies to ascertain whether or not the patient has sensorineural hearing loss. For instance, a subset frequencies, or a single frequency is tested to identify a patient with sensorineural hearing loss.

To determine the hearing threshold, the volume of the pure tone is altered to determine the lowest level of stimuli that the patient is able to detect. The lowest level of stimuli (corresponding to the quietest sound) is the pure tone hearing threshold at a given frequency. The pure tone threshold is typically measured in a patient using according decibels in hearing level (dB HL) on an audiometer. However, hearing thresholds may also be determined using other methods known to the person skilled in the art. For example, hearing function is measured by Auditory Brainstem Response (ABR) testing or Auditory Steady State Response (ASSR) testing. Other tests can also be used to determine hearing function in a patient. For instance, Distortion product otoacoustic emissions (DPOAEs) can be used to measure outer hair cell function and loss and is used in differential diagnosis of hearing loss arising from hair cell loss from hearing loss associated with higher level processing (e.g. auditory neuropathy).

Pure tone thresholds are plotted on a graph to produce an audiogram for the patient.

Pure tone thresholds measured across different frequencies may also be averaged to provide a pure tone average. For instance, a patient that has pure tone hearing thresholds of 50 dB HL at 0.5 Hz, 60 dB HL at 1 kHz, 65 dB HL at 2 kHz and 70 dB at 4 kHz would have a pure tone average of 61.25 dB HL, when measured across 0.5 kHz, 1 kHz, 2 kHz and 4 kHz.

Pure tone averages are calculated across different frequencies. Pure tone thresholds at any subset of frequencies are used to calculate pure tone averages. In some embodiments, the average of the patient hearing threshold is measured across 0.5 kHz, 1 kHz, 2 kHz and 4 kHz. In some embodiments, pure tone average is measured across 4 kHz, 6 kHz and 8 kHz. Measurement of pure tone average across 4 kHz, 6 kHz and 8 kHz is useful when seeking to assess the patient's hearing function at the higher frequencies within the standard audiometric frequencies.

Sensorineural hearing loss can be categorized according to its severity. The severity of hearing loss is determined by the hearing levels at which a threshold level is obtained in a patient by pure tone audiometry. Severity of hearing loss is classified according to hearing thresholds using the following definitions:

Normal: 25 dB HL or less

Mild: at least 25 dB HL and no more than 40 dB HL

Moderate: at least 40 dB HL and no more than 55 dB HL

Moderately Severe: at least 55 dB HL and no more than 70 dB HL

Severe: at least 70 dB HL and no more than 90 dB HL

Profound: at least 90 dB HL or more

These measures of severity are standard measures in the field (see Goodman, A. (1965). Reference zero levels for pure tone audiometer. ASHA, 7, 262-263). In some embodiments, the severity of hearing loss is classified according to a patient's hearing threshold at a single frequency (for example, 0.25 kHz, 0.5 kHz, 1 kHz, 2 kHz, 3 kHz, 4 kHz, 6 kHz or 8 kHz). For instance, a patient may have mild hearing loss at 8 kHz, and normal hearing at the other standard audiometric frequencies. In some embodiments, the severity of hearing loss is classified according to pure tone average, when measured across a subset of frequencies. In certain such embodiments, the severity of hearing loss is classified according to the pure tone average across 0.5 kHz, 1 kHz, 2 kHz and 4 kHz. For example, a patient may have moderate hearing loss according to their pure tone average across 0.5 kHz, 1 kHz, 2 kHz and 4 kHz, but have moderately severe hearing loss at a single frequency (e.g. 8 kHz). In other embodiments, the severity of hearing loss is classified according to the pure tone average across 4 kHz, 6 kHz and 8 kHz.

A patient that has hearing threshold of 25 dB HL or less at standard audiometric frequencies (i.e. 0.25 kHz, 0.5 kHz, 1 kHz, 2 kHz, 3 kHz, 4 kHz, 6 kHz and 8 kHz) has normal hearing. The patient's audiogram is also a normal audiogram.

### Word Recognition Tests

Alternatively, or in addition to pure tone audiometry, hearing loss is assessed using a word recognition test. A word recognition test measures the patient's ability to correctly identify a word, thereby providing a measure of sound intelligibility (in particular, speech intelligibility) that may not be provided by pure tone audiometry. In some embodiments, a word recognition score is used to determine the patient's ability to correctly identify words prior to treatment.

A standard word recognition in quiet test, also referred to herein as a standard word recognition test, is a test administered by an audiologist that measures a patient's speech intelligibility in recognizing words in a quiet environment. A quiet environment is an environment with little to no background noise.

A standard word recognition test is used to determine a person's ability to recognize words selected from a word list and presented to the patient at a given decibel (dB) level. In some embodiments, the standard word recognition test is used to determine a patient's ability to recognize words at more than one decibel level.

In some embodiments, the standard word recognition test assesses the patient's ability to identify 50 words. However, the number of words presented to the patient is more or less than 50. For example, in some embodiments, the standard word recognition test is for 25 words. In other embodiments, the standard word recognition test is for 10 words.

A standard word recognition test is used to generate a standard word recognition (%) score which is calculated using the formula:

$$standard word recogntion score ( \% ) = 100 \times ( \frac{words recognised in standard word recognition test}{total words} )$$

In some embodiments, the standard word recognition score is expressed as the number of words that are correctly recognized in the test.

In some embodiments, a list of words is administered to each ear, and a standard word recognition score is calculated for each ear. Herein the results of the standard word recognition score refer to the ear that has been/will be treated.

A standard word recognition test is carried out using any list of words. However, standard word lists are typically used in a standard word recognition test. In some embodiments, each test word is embedded in a carrier phrase. Example of carrier phrases are: “Say the word ______ again”, “You will say ______”, or “Say the word ______”.

In some embodiments, the standard word recognition test is the Maryland consonant-vowel nucleus-consonant (CNC) word test. The Maryland CNC word test has been described, for example, in Mendel, L. L., Mustain, W. D., & Magro, J. (2014). Normative data for the Maryland CNC Test. Journal of the American Academy of Audiology, 25, 775-781.

The Maryland CNC word test is a standard word recognition test that uses phonemically balanced word lists comprising words that are consonant-nucleus-consonant (CNC) monosyllables. These CNC lists are balanced so that each initial consonant, each vowel, and each final consonant appears with the same frequency within each list. The Maryland CNC test has 10 lists of 50 words.

In some embodiments, the Maryland CNC Test uses words from Lehiste and Peterson's phonemically balanced word lists, all of which were CNC monosyllables, for example as described in Lehiste I, Peterson G E. (1959) Linguistic considerations in the study of speech intelligibility. Journal of the Acoustical Society of America 31(3): 280-286.

In some embodiments, the Maryland CNC Test uses words from revised CNC lists that eliminate rare literary words and proper names, for example as described in Peterson G E, Lehiste I. (1962) Revised CNC lists for auditory tests. Journal of Speech and Hearing Disorders 27:62-70.

In some embodiments, the Maryland CNC Test uses words from modified CNC word lists that take into consideration the effects of coarticulation, where the acoustic properties of phonemes are influenced by those phonemes that immediately precede and follow them, for example as described in Causey G D, Hood L J, Hermanson C L, Bowling L S. (1984) The Maryland CNC Test: normative studies. Audiology 23(6): 552-568. The words of the Maryland CNC test are spoken within the carrier phrase: ‘Say the ______ again,’

In some embodiments, the standard word recognition test is the C.I.D Auditory Test W-22 (CID W-22) test. The CID W-22 test has been described, for example, in Hirsh, I. J., Davis, H. Silverman, S. R., Reynolds, E. G. Eldert, E., & Benson, R. W. (1952). Development of Materials for Speech Audiometry. Journal of Speech, Language, and Hearing Research, 17(3), 321-337.

The CID W-22 test uses 200 monosyllabic words which are divided into four lists of 50 words each. Each list is phonetically balanced. The speech sounds within the list occur with the same relative frequency as they do in a representative sample of English speech. There are three criteria for the vocabulary in the phonetically balanced word lists. First, all the words must be one-syllable words with no repetition of words in the different lists. Second, any word chosen should be a familiar word. This second criterion is to minimize the effect of differences in the educational background of subjects. Third, the phonetic composition of each word list should correspond to that of English as a whole as closely as possible. The words of the CID W-22 test are spoken with the carrier phrase: “You will say ______”.

In some embodiments the standard word recognition test is the NU No. 6 test. The NU No. 6 has been described, for example, in Tillman, T. W., & Carhart, R. (1966). An expanded test for speech discrimination utilizing CNC monosyllabic words: Northwestern University Auditory Test No. 6. Northwestern Univ Evanston II Auditory Research Lab.

In some embodiments, the NU No. 6 test uses 4 lists of 50 words, for example, as described in Table 28-2 of Tillman, T. W., & Carhart, R. (1966). The words of the NU No. 6 test are spoken with the carrier phrase: “Say the word ______”.

In some embodiments the standard word recognition test is the Maryland CNC test, using the words list and carrier phrases as defined in Causey G D, Hood L J, Hermanson C L, Bowling L S. (1984) The Maryland CNC Test: normative studies. Audiology 23(6): 552-568. In certain such embodiments, the word signal is provided to the patient at 40 dB above speech perception level.

### Words-in-Noise (WIN) Test

A “Words-in-Noise (WIN) Test” is a test administered by an audiologist to measure a patient's speech intelligibility in recognizing words in the presence of background noise.

The WIN test consists of administering words to an ear at a varying signal-to-noise ratio (SNR) level. The signal-to-noise ratio is the ratio of the strength of the signal carrying information (e.g. the test word signal), relative to the signal of interference (e.g. noise), and is typically expressed in decibels. In some embodiments, the background noise is multi-talker babble at a fixed decibel level.

In some embodiments the multi-talker babble is comprised of six talkers (three female, three male) at a fixed level, for example, as described in Wilson, R. H., Abrams, H. B., & Pillion, A. L. (2003). A word-recognition task in multi-talker babble using a descending presentation mode from 24 dB to 0 dB signal to babble. Journal of Rehabilitation Research and Development, 40(4), 321-328.

In some embodiments, the background noise is maintained at a fixed decibel level, and the variation in the SNR decibel level is achieved by varying the decibel level of the test word signal. The SNR decibel level is therefore the SNR above the background noise. For example if the level of multi-talker babble is fixed at 70 dB SPL, and the level of the test word signal varied from 70 dB SPL to 94 dB SPL, this would give a SNR decibel level variation of 0 dB to 24 dB.

In some embodiments, the test words that are used are from any list described herein for the word recognition tests. In some embodiments, the word-in-noise test is for 70 words. In other embodiments, the words-in-noise test is for 35 words.

In some embodiments, the test consists of administering 35 or 70 monosyllabic words from the NU No. 6 word lists. The test words are spoken with the carrier phrase: “Say the word ______”.

In some embodiments, the WIN test is administered in a descending-level SNR paradigm. In these embodiments, the test words at the high SNR decibel level are presented first, followed by test words at gradually lower SNR decibel levels, with words at the lowest SNR decibel level administered last. The high SNR decibel level is the easiest setting for the patient to identify the signal words. The low SNR decibel levels is the most difficult setting for the patient to identify the signal words. In other embodiments, the WIN test is administered in a randomized-level SNR paradigm. In these embodiments, the test words are presented at different SNR decibel levels in a randomized order.

In some embodiments the SNR decibel level of the test words varies from 24 dB SNR (easiest condition) to 0 dB SNR (most difficult condition) in 4 dB decrements, for a total of seven SNR levels (i.e. 24 dB SNR, 20 dB SNR, 16 dB SNR, 12 dB SNR, 8 dB SNR, 4 dB SNR and 0 dB SNR).

In some embodiments the WIN test consists of administering 70 monosyllabic words from the NU No. 6 word lists, where the SNR decibel level of the test words varies from 24 dB SNR (easiest condition) to 0 dB SNR (most difficult condition) in 4 dB decrements, for a total of seven SNR levels (i.e. 24 dB SNR, 20 dB SNR, 16 dB SNR, 12 dB SNR, 8 dB SNR, 4 dB SNR and 0 dB SNR). In this embodiment, the level of multi-talker babble is fixed at 70 dB SPL, and the level of the test word signal varies from 70 dB SPL to 94 dB SPL.

The ‘words-in-noise’ test is used to generate a words-in-noise score.

In some embodiments the words-in-noise score is given as a percentage of the total correct words recognized by the patient in the test and calculated using the formula:

$$words n noise score ( \% ) = 100 \times ( \frac{words recognised in standard words in noise test}{total words} )$$

### Methods of Use

In certain embodiments, the present disclosure relates to inducing, promoting, or enhancing the growth, proliferation or regeneration of inner ear tissue, particularly inner ear supporting cells and hair cells. Some embodiments relate to methods for controlled proliferation of stem cells comprising an initial phase of inducing sternness while inhibiting differentiation and a subsequent phase of differentiation of the stem cells into tissue cells.

When cochlear supporting cell or vestibular supporting cell populations are treated with an agent in accordance to the methods of the invention, whether the population is in vivo or in vitro, the treated supporting cells exhibit stem-like behavior in that the treated supporting cells have the capacity to proliferate and differentiate and, more specifically, differentiate into cochlear hair cells or vestibular hair cells. In some instances, an agent induces and maintains the supporting cells to produce daughter stem cells that can divide for many generations and maintain the ability to have a high proportion of the resulting cells differentiate into hair cells. In certain embodiments, the proliferating stem cells express stem cell marker(s) selected from one or more of Lgr5, Sox2, Opem1, Phex, lin28, Lgr6, cyclin D1, Msx1, Myb, Kit, Gdnf3, Zic3, Dppa3, Dppa4, Dppa5, Nanog, Esrrb, Rex1, Dnmt3a, Dnmt3b, Dnmt31, Utf1, Tcl1, Oct4, Klf4, Pax6, Six2, Zic1, Zic2, Otx2, Bmi1, CDX2, STAT3, Smad1, Smad2, smad2/3, smad4, smad5, and smad7. In some embodiments, the proliferating stem cells express stem cell marker(s) selected from one or more of Lgr5, the

In some embodiments, the methods are used to maintain, or even transiently increase sternness (i.e. self-renewal) of a pre-existing supporting cell population prior to significant hair cell formation. In some embodiments, the pre-existing supporting cell population comprises inner pillar cells, outer pillar cells, inner phalangeal cells, Deiter cells, Hensen cells, Boettcher cells, and/or Claudius cells. Morphological analyses with immunostaining (including cell counts) and lineage tracing across a Representative Microscopy Samples are used to confirm expansion of one or more of these cell-types. In some embodiments, the pre-existing supporting cells comprise Lgr5+ cells. Morphological analyses with immunostaining (including cell counts) and qPCR and in situ RNA hybridization is used to confirm Lgr5 upregulation amongst the cell population.

Advantageously, methods described herein can achieve these goals without the use of genetic manipulation. Germ-line manipulation used in many academic studies is not a therapeutically desirable approach to treating hearing loss. In general, the therapy involves the administration of a small molecule, peptide, antibody, or other non-nucleic acid molecule or nucleic acid delivery vector unaccompanied by gene therapy. In certain embodiments, the therapy involves the administration of a small organic molecule. In some instances, hearing protection or restoration is achieved through the use of a (non-genetic) therapeutic that is injected in the middle ear and diffuses into the cochlea.

The cochlea relies heavily on all present cell types, and the organization of these cells is important to their function. For instance, supporting cells play an important role in neurotransmitter cycling and cochlear mechanics. Thus, maintaining a rosette patterning within the organ of Corti is important for maintaining function. Cochlear mechanics of the basilar membrane activate hair cell transduction. Due to the high sensitivity of cochlear mechanics, it is also desirable to avoid masses of cells. In all, maintaining proper distribution and relation of hair cells and supporting cells along the basilar membrane, even after proliferation, is likely a desired feature for hearing as supporting cell function and proper mechanics is necessary for normal hearing.

In some embodiments, the cell density of hair cells in a cochlear cell population is expanded in a manner that maintains, or even establishes, the rosette pattern characteristic of cochlear epithelia.

In certain embodiments, the cell density of hair cells is increased in a population of cochlear cells comprising both hair cells and supporting cells. The cochlear cell population can be an in vivo population (i.e. comprised by the cochlear epithelium of a subject) or the cochlear cell population is an in vitro (ex vivo) population. If the population is an in vitro population, the increase in cell density is determined by reference to a Representative Microscopy Sample of the population taken prior and subsequent to any treatment. If the population is an in vivo population, the increase in cell density is determined indirectly by determining an effect upon the hearing of the subject with an increase in hair cell density correlating to an improvement in hearing.

In some embodiments, supporting cells placed in a Stem Cell Proliferation Assay in the absence of neuronal cells form ribbon synapses.

In a native cochlea, patterning of hair cells and supporting cells occurs in a manner parallel to the basilar membrane. In some embodiments, the proliferation of supporting cells in a cochlear cell population is expanded in a manner that the basilar membrane characteristic of cochlear epithelia.

In some embodiments, the number of supporting cells in an initial cochlear cell population is selectively expanded by treating the initial cochlear cell population with a composition of the present disclosure to form an intermediate cochlear cell population, wherein the ratio of supporting cells to hair cells in the intermediate cochlear cell population exceeds the ratio of supporting cells to hair cells in the initial cochlear cell population. The expanded cochlear cell population is, for example, an in vivo population, an in vitro population or even an in vitro explant. In some embodiments, the ratio of supporting cells to hair cells in the intermediate cochlear cell population exceeds the ratio of supporting cells to hair cells in the initial cochlear cell population. For example, in some embodiments, the ratio of supporting cells to hair cells in the intermediate cochlear cell population exceeds the ratio of supporting cells to hair cells in the initial cochlear cell population by a factor of 1.1, 1.5, 2, 3, 4, 5 or more. In some instances, the capacity of a composition to expand a cochlear cell population is be determined by means of a Stem Cell Proliferation Assay.

In some embodiments, the number of stem cells in a cochlear cell population is expanded to form an intermediate cochlear cell population by treating a cochlear cell population with a composition of the present disclosure wherein the cell density of stem cells in the intermediate cochlear cell population exceeds the cell density of stem cells in the initial cochlear cell population. The treated cochlear cell population is, for example, an in vivo population, an in vitro population or even an in vitro explant. In one such embodiment, the cell density of stem cells in the treated cochlear cell population exceeds the cell density of stem cells in the initial cochlear cell population by a factor of at least 1.1, 1.25, 1.5, 2, 3, 4, 5 or more. In vitro cochlear cell populations may expand significantly more than in vivo populations; for example, in certain embodiments the cell density of stem cells in an expanded in vitro population of stem cells is at least 4, 5, 6, 7, 8, 9, 10, 15, 20, 25, 30, 35, 40, 45, 50, 75, 100, 200, 300, 400, 500, 600, 700, 800, 900, 1000, 2000 or even 3000 times greater than the cell density of the stem cells in the initial cochlear cell population. In some instances, the capacity of a composition to expand a cochlear cell population is determined by means of a Stem Cell Proliferation Assay.

In some embodiments, a cochlear supporting cell population or a vestibular supporting cell population is treated with a composition of the present disclosure to increase the Lgr5 activity of the population. For example, in some instances an epigenetic agent and a Wnt agonist has the capacity to increase and maintain the Lgr5 activity of an in vitro population of cochlear supporting cells or vestibular supporting cells by factor of at least 1.2, 1.5, 2, 3, 4, 5, or more. In some embodiments, the epigenetic agent and a Wnt agonist has the capacity to increase the Lgr5 activity of an in vitro population of cochlear supporting cells or vestibular supporting cells by factor of 2, 3, 5 10, 100, 500, 1000, 2000 or even 3000. Increases in Lgr5 activity may also be observed for in vivo populations but the observed increase is less than in vitro populations. In some instances, the epigenetic agent and a Wnt agonist inhibitor has the capacity to increase the Lgr5 activity of an in vivo population of cochlear supporting cells or vestibular supporting cells by about or at least about 5%, 10%, 20%, 30% or more. In some instances, the capacity of the epigenetic agent and a Wnt agonist for such an increase in Lgr5 activity is demonstrated, for example, in an In Vitro Lgr5+ Activity Assay, and in an in vivo population is demonstrated, for example, in an In Vivo Lgr5+ Activity Assay, as measured by isolating the organ and performing morphological analyses using immunostaining, endogenous fluorescent protein expression of Lgr5, and qPCR for Lgr5.

In some embodiments, the epigenetic agent in combination with a Wnt agonist has the capacity to increase the Lgr5 Activity of an in vitro population of cochlear supporting cells or vestibular supporting cells by a factor of 10, 20, 30, 40, 50, 75, 100 or 200% compared to a Wnt agonist alone as measured for example in an In Vitro Lgr5+ Activity Assay.

In some embodiments, the epigenetic agent in combination with CHIR99021 has the capacity to increase the Lgr5 Activity of an in vitro population of cochlear supporting cells or vestibular supporting cells by a factor of 10, 20, 30, 40, 50, 75, 100 or 200% compared to CHIR99021 in combination with VPA, as measured for example in an In Vitro Lgr5+ Activity Assay.

In some embodiments, the epigenetic agent in combination with a Wnt agonist has the capacity to increase the Lgr5 proliferation of an in vitro population of cochlear supporting cells or vestibular supporting cells by factor of 10, 20, 30, 40, 50, 75, 100 or 200% compared to a Wnt agonist alone as measured for example in a in a in a Stern Cell Proliferation Assay.

In some embodiments, the epigenetic agent in combination with a Wnt agonist has the capacity to increase the Lgr5 proliferation of an in vitro population of cochlear supporting cells or vestibular supporting cells by factor of 10, 20, 30, 40, 50, 75, 100 or 200% compared to a Wnt agonist in combination with a VPA as measured for example in a in a in a Stem Cell Proliferation Assay.

In some embodiments, the epigenetic agent in combination with a Wnt agonist and VPA has the capacity to increase the Lgr5 proliferation of an in vitro population of cochlear supporting cells or vestibular supporting cells by factor of 10, 20, 30, 40, 50, 75, 100 or 200% compared to a in combination with a VPA as measured for example in a in a in a Stem Cell Proliferation Assay.

In addition to increasing the Lgr5 activity of the population, the number of Lgr5+ supporting cells in a cochlear or vestibular cell population is increased by treating a cochlear or vestibular cell population containing Lgr5+ supporting cells (whether in vivo or in vitro) with a composition of the present disclosure. In general, the cell density of the stem/progenitor supporting cells may expand relative to the initial cell population via one or more of several mechanisms. For example, in some embodiments, newly generated Lgr5+ supporting cells is generated that have increased stem cell propensity (i.e. greater capacity to differentiate into hair cell). By way of further example, in some embodiments no daughter Lgr5+ cells are generated by cell division, but pre-existing Lgr5+ supporting cells are induced to differentiate into hair cells. By way of further example, in some embodiments no daughter cells are generated by cell division, but Lgr5− supporting cells are activated to a greater level of Lgr5 activity and the activated supporting cells are then able to differentiate into hair cells. Regardless of the mechanism, in some embodiment a composition of the present disclosure (e.g. a composition comprising an epigenetic agent and a Wnt agonist and optionally a second epigenetic agent) has the capacity to increase the cell density of Lgr5+ supporting cells in an in vitro isolated cell population of cochlear supporting cells or vestibular supporting cells by factor of at least 5, 10, 50, 100, 500, 1000, or 2000. Increases in the cell density of Lgr5+ supporting cells may also be observed for in vivo populations but the observed increase is somewhat more modest. For example, in some embodiments the composition has the capacity to increase the cell density of Lgr5+ supporting cells in an in vivo population of cochlear supporting cells or vestibular supporting cells by about or at least about 5%, 10%, 20%, 30% or more. The capacity of the composition (for such an increase in Lgr5+ supporting cells in an in vitro population is demonstrated, for example, in a Stem Cell Proliferation Assay or in an appropriate in vivo assay. In some embodiments, a composition of the present disclosure has the capacity to increase the number of Lgr5+ cells in the cochlea by inducing expression of Lgr5 in cells with absent or low detection levels of the protein, while maintaining Native Morphology. In some embodiments, a composition has the capacity to increase the number of Lgr5+ cells in the cochlea or vestibular organ by inducing expression of Lgr5 in cells with absent or low detection levels of the protein, while maintaining Native Morphology and without producing Cell Aggregates.

Included in the invention are methods of increasing proliferation of a Lgr5+ cochlear supporting cell by contacting a cochlear supporting cell with an epigenetic agent and a Wnt agonist. Optionally, the cell is further contacted with an epigenetic agent such as an HDAC inhibitor. In some embodiments, the HDAC inhibitor is VPA.

Included in the invention are methods of increasing proliferation of a vestibular supporting cell by contacting a vestibular supporting cell with an epigenetic agent and a Wnt agonist. Optionally, the cell is further contacted with an epigenetic agent such as an HDAC inhibitor. In some embodiments, the HDAC inhibitor is VPA.

In the various methods Lgr5+ cochlear cell or vestibular cell proliferation is increased compared to a vehicle control.

In some embodiments, the epigenetic agent and the Wnt agonist increases Lgr5+ cochlear supporting cell or vestibular supporting cell proliferation by at least 10, 20, 30, 40, 50, 60, 70, 80, 90, 100, 150, 200, 250, 300, 350, 400, 450, or 500% or more (or at least about 1.1, 1.2, 1.3, 1.4, 1.5, 1.6, 1.7, 1.8, 1.9, 2, 3, 4, 5, 6, 7, 8, 9, 10, 15, 20, 30, 40, 50, 60, 70, 80, 90, 100, 200, 500, 1000-fold or more), relative to a vehicle control.

In some embodiments, the epigenetic agent and the Wnt agonist in combination with a second epigenetic agent increases Lgr5+ cochlear supporting cell or vestibular supporting cell proliferation by at least 10, 20, 30, 40, 50, 60, 70, 80, 90, 100, 150, 200, 250, 300, 350, 400, 450, or 500% more (or at least about 1.1, 1.2, 1.3, 1.4, 1.5, 1.6, 1.7, 1.8, 1.9, 2, 3, 4, 5, 6, 7, 8, 9, 10, 15, 20, 30, 40, 50, 60, 70, 80, 90, 100, 200, 500, 1000-fold or more) relative to a Wnt agonist alone in a Stem Cell Proliferation Assay.

In some embodiments, the epigenetic agent and the Wnt agonist in combination with a second epigenetic agent increases Lgr5+ cochlear supporting cell or vestibular supporting cell proliferation by at least 10, 20, 30, 40, 50, 60, 70, 80, 90, 100, 150, 200, 250, 300, 350, 400, 450, or 500% more (or at least about 1.1, 1.2, 1.3, 1.4, 1.5, 1.6, 1.7, 1.8, 1.9, 2, 3, 4, 5, 6, 7, 8, 9, 10, 15, 20, 30, 40, 50, 60, 70, 80, 90, 100, 200, 500, 1000-fold or more) relative to Wnt agonist in combination with VPA in a Stem Cell Proliferation Assay.

In some embodiments, the epigenetic agent and the Wnt agonist increases Lgr5+ cochlear supporting cell or vestibular supporting cell proliferation by at least 10, 20, 30, 40, 50, 60, 70, 80, 90, 100, 150, 200, 250, 300, 350, 400, 450, or 500% or more (or at least about 1.1, 1.2, 1.3, 1.4, 1.5, 1.6, 1.7, 1.8. 1.9, 2, 3, 4, 5, 6, 7, 8, 9, 10, 15, 20, 30, 40, 50, 60, 70, 80, 90, 100, 200, 500, 1000-fold or more), relative to a Wnt agonist alone, as measured in a Stem Cell Proliferation Assay.

In some embodiments, the epigenetic agent and the Wnt agonist increases Lgr5+ cochlear supporting cell or vestibular supporting cell proliferation by at least 10, 20, 30, 40, 50, 60, 70, 80, 90, 100, 150, 200, 250, 300, 350, 400, 450, or 500% or more (or at least about 1.1, 1.2, 1.3, 1.4, 1.5, 1.6, 1.7, 1.8, 1.9, 2, 3, 4, 5, 6, 7, 8, 9, 10, 15, 20, 30, 40, 50, 60, 70, 80, 90, 100, 200, 500, 1000-fold or more), relative to a Wnt agonist in combination with VPA, as measured in a Stem Cell Proliferation Assay.

Also included are methods for expanding a population of cochlear cells in a cochlear tissue comprising a parent population of cells by contacting the cochlear tissue with an epigenetic agent and a Wnt agonist to form an expanded population of cells in the cochlear tissue. Optionally, the cell is further contacted with a second epigenetic agent such as an HDAC inhibitor such as an class I HDAC inhibitor In some embodiment, the class I HDAC inhibitor is a short chain carboxylic acid such as for example, valproic acid (VPA).

The epigenetic agent and the Wnt agonist (optionally in combination with a second epigenetic agent) is capable of (i) forming a proliferation assay final cell population from a proliferation assay initial cell population over a proliferation assay time period in a stem cell proliferation assay, and/or (ii) forming a differentiation assay final cell population from a differentiation assay initial cell population over a differentiation assay time period in a stem cell differentiation assay wherein: (a) the proliferation assay initial cell population has (i) a proliferation assay initial number of total cells, (ii) a proliferation assay initial number of Lgr5+ cells, (iii) a proliferation assay initial number of hair cells, (iv) a proliferation assay initial Lgr5+ cell fraction that equals the ratio of the proliferation assay initial number of Lgr5+ cells to the proliferation assay initial number of total cells, and (v) a proliferation assay initial hair cell fraction that equals the ratio of the proliferation assay initial number of hair cells to the proliferation assay initial number of total cells; (b) the proliferation assay final cell population has (i) a proliferation assay final number of total cells, (ii) a proliferation assay final number of Lgr5+ cells, (iii) a proliferation assay final number of hair cells, (iv) a proliferation assay final Lgr5+ cell fraction that equals the ratio of the proliferation assay final number of Lgr5+ cells to the proliferation assay final number of total cells and (v) a proliferation assay final hair cell fraction that equals the ratio of the proliferation assay final number of hair cells to the proliferation assay final number of total cells; (c) the differentiation assay initial cell population has (i) a differentiation assay initial number of total cells, (ii) a differentiation assay initial number of Lgr5+ cells, (iii) a differentiation assay initial number of hair cells, (iv) a differentiation assay initial Lgr5+ cell fraction that equals the ratio of the differentiation assay initial number of Lgr5+ cells to the differentiation assay initial number of total cells, and (v) a differentiation assay initial hair cell fraction that equals the ratio of the differentiation assay initial number of hair cells to the differentiation assay initial number of total cells; (d) the differentiation assay final cell population has (i) a differentiation assay final number of total cells, (ii) a differentiation assay final number of Lgr5+ cells, (iii) a differentiation assay final number of hair cells, (iv) a differentiation assay final Lgr5+ cell fraction that equals the ratio of the differentiation assay final number of Lgr5+ cells to the differentiation assay final number of total cells, and (v) a differentiation assay final hair cell fraction that equals the ratio of the differentiation assay final number of hair cells to the differentiation assay final number of total cells; (e) the proliferation assay final number of Lgr5+ cells exceeds the proliferation assay initial number of Lgr5+ cells by a factor of at least 10; and/or (f) the differentiation assay final number of hair cells is a non-zero number.

The invention also includes methods of producing an expanded population of Lgr5+ cochlear cells by contacting the cell population with an epigenetic agent and Wnt agonist to form an expanded population of cells in the cochlear tissue. Optionally, the cell is further contacted with a second epigenetic agent such as an HDAC inhibitor. In some embodiments, the HDAC inhibitor is VPA.

The expanded population is capable of differentiating into hair cells as measured in a stem cell differentiation assay.

In some embodiments, the cochlear cell is in a cochlear tissue. In some embodiments, the cochlear tissue is in a subject.

Some embodiments relate to methods of treating a subject who has, or is at risk for developing, hearing loss or reduced auditory function. The prophylaxis and/or treatment of acute and chronic ear disease and hearing loss, dizziness and balance problems especially of sudden hearing loss, acoustic trauma, hearing loss due to chronic noise exposure, presbycusis, trauma during implantation of the inner ear prosthesis (insertion trauma), dizziness due to diseases of the inner ear area, dizziness related and/or as a symptom of Meniere's disease, vertigo related and/or as a symptom of Meniere's disease, tinnitus, hyperacusis and hearing loss due to antibiotics and cytostatics and other drugs.

Some embodiments include methods to prevent, reduce, or treat the incidence and/or severity of inner ear disorders and hearing impairments involving inner ear tissue, particularly inner ear hair cells, their progenitors, and optionally, the stria vascularis, and associated auditory nerves. Of particular interest are those conditions that lead to permanent hearing loss where reduced number of hair cells are responsible and/or decreased hair cell function. Also of interest are those arising as an unwanted side-effect of ototoxic therapeutic drugs including cisplatin and its analogs, aminoglycoside antibiotics, salicylate and its analogs, or loop diuretics.

Hearing loss or reduced auditory function is treated or prevented in a subject by contacting a Lgr5+ cochlear cell or administering to the subject an epigenetic agent and Wnt agonist to form an expanded population of cells in the cochlear tissue. Optionally, the cell is further contacted with a second epigenetic agent such as an HDAC inhibitor. In some embodiments, the HDAC inhibitor is VPA.

In various embodiments the epigenetic agent and Wnt agonist and optionally, the one or more additional epigenetic agents are administered to the subject systemically or locally. Systemic administration includes, but is not limited, to oral or parenteral administration. Parenteral routes include for example intramuscular (IM), subcutaneous (SC) and intravenous (IV). Local administration includes for example, intratympanic or intracochlear administration. More specific methods of local delivery are described herein. In some embodiments, both the epigenetic agent and Wnt agonist are administered locally. In other embodiments, both the epigenetic agent and Wnt agonist are administered systemically. In some embodiments the epigenetic agent is administered locally and the Wnt agonist is administered systemically. In other embodiments the epigenetic agent is administered systemically and the Wnt agonist is administered locally.

In some embodiments, the epigenetic agent and Wnt agonist are administered at the same time. In other embodiments, the epigenetic agent and Wnt agonist are administered at different times. In some embodiments the epigenetic agent is administered a period of time before the Wnt agonist. In other embodiments, the epigenetic agent is administered at a period of time after the Wnt agonist. For example, the epigenetic agent is administered 1, 2, 3, 4, 5, 6, 7, 8, 9, 10, 12, 13, 14, 14, 15, 17, 18, 19, 20, 21, 22, 23, 24 hours or 1, 2, 3, 4, 5, 6, 7 or more days before the Wnt agonist. Alternatively, the epigenetic agent is administered 1, 2, 3, 4, 5, 6, 7, 8, 9, 10, 12, 13, 14, 14, 15, 17, 18, 19, 20, 21, 22, 23 or 24 hours or 1, 2, 3, 4, 5, 6, 7 or more days before the Wnt agonist after the Wnt agonist.

Hearing loss or reduced auditory function is treated or prevented utilizing the various methods described herein to increase Lgr5+ cochlear cell proliferation. The cochlear cell is contacted with an epigenetic agent and Wnt agonist at a “cell effective concentration” to form an expanded population of cells in the cochlear tissue. Optionally, the cell is further contacted with a second epigenetic agent such as an HDAC inhibitor. In some embodiments, the HDAC inhibitor is VPA.

A “cell effective concentration” is the minimum concentration of the compound that induces at least an 1.1, 1.2, 1.3, 1.4, 1.5, 1.6, 1.7, 1.8, 1.9, 2, 3, 4, 5, 6, 7, 8, 9, 10, 15, 20, 30, 40, 50, 60, 70, 80, 90, 100, 200, 500, 1000-fold or more in gene expression and/or about a 1.5-fold increase in number of Lgr5+ cells in a Stem Cell Proliferation Assay compared to a vehicle control.

In some embodiments, the Lgr5+ cochlear cell is contacted in vitro with the compound(s) at the “cell effective concentration”, such as for example, in a cell culture (and then implanted into the cochlea). In other embodiments, the Lgr5+ cochlear cell is contacted with the compound(s) at the “cell effective concentration”, in situ (i.e. within the cochlea). In some embodiments, sufficient compound is delivered to achieve the “cell effective concentration” throughout the speech region of the human cochlea. In order to achieve this target concentration, a higher concentration of drug is instilled in the cochlea and diffuse throughout the speech region. In other embodiments, the Lgr5+ cochlear cell is contacted with the compound(s) at 2, 3, 4, 5, 10, 20, 50, 100, 200, 300, 400, 500, 600, 700, 800, 900, 1000-fold more than the “cell effective concentration”, in situ (i.e. within the cochlea).

Alternatively, hearing loss or reduced auditory function is treated by administering the compound(s) at the “formulation effective concentration”. A “formulation effective concentration” is a higher concentration than the “cell effective formulation”. For example, the “formulation effective concentration” is at least about 100 to 5000 fold higher than the “cell effective concentration”, or about 20 100, 250, 500, 750, 1000, 1250, 1500, 1750, 2000 fold higher than the “cell effective concentration”, or about 100, 200, 300, 400, 500, 600, 700, 800, 900 or 1000 fold higher than the “cell effective concentration”. Typically, the “formulation effective concentration” is at least about 1000 fold higher than the “cell effective concentration”.

Alternatively, hearing loss or reduced auditory function is treated by administering the compound(s) at a set daily dose.

The compound(s) are formulated at the “cell effective concentration” and the “formulation effective concentration” as described supra.

In some embodiments, the “cell effective concentration” of the compound(s) is about 0.01 pM to 1000 nM, about 1 pM to 100 nM, about 10 pM to 10 nM, about 1 pM to 10 pM, about 10 nM to 100 nM, about 100 nM to 1000 nM, about 1 nM to 10 nM, 0.01 μM to 1000 μM, about 1 μM to 100 μM, about 10 μM to 10 μM, about 1 μM to 1 mM, or about 10 mM to 100 mM.

In some embodiment the compound is administered to the subject systemically at a daily dose of about 0.01 mg to 1000 mg/day; about 0.01 mg to 500 mg/day; about 0.01 mg to 250 mg/day; about 0.01 mg to 100 mg/day; about 0.01 mg to 50 mg/day; about 0.01 mg to 25 mg/day; about 0.01 mg to 10 mg/day; about 0.01 mg to 5 mg/day; 0.1 mg to 100 mg/day; about 0.1 mg to 50 mg/day; about 0.01 mg to 2.5 mg/day; about 0.01 mg to 10 mg/day; about 0.01 mg to 5 mg/day; about 0.01 mg to 2.5 mg/day; about 0.1 mg to 10 mg/day; about 0.1 mg to 5 mg/day about 0.1 mg to 4 mg/day; about 0.1 mg to 3 mg/day; about 0.1 mg to 2 mg/day; about 0.1 mg to 2 mg/day or about 1 mg to 5 mg/day.

In some embodiments, compound is administered to the subject at a concentration ratio of about 0.001 to 10 fold relative to an FDA approved concentration or about 0.1 to 50 fold relative to an FDA approved concentration, or about 0.1 to 5 fold relative to an FDA approved, or about 1 to 5 fold relative to an FDA approved concentration. In some embodiments, compound administered to the subject at about 0.01×. 0.1×, 2×, 3×, 5× or 1.0×, relative to an FDA approved concentration.

In some embodiments, the epigenetic agent “cell effective concentration” is about 0.01 pM to 100 μM, about 0.1 pM to 10 μM, about 1 pM to 1 μM, about 0.01 μM to 10 μM, about 0.1 μM to 10 μM, about 1 μM to 10 μM, 10 μM to 100 μM, or about 100 μM to 1000 mM.

In some embodiments, the epigenetic agent “formulation effective concentration” is about 0.01 μM to 100 mM, about 0.1 μM to 10 mM, about 1 μM to 1 mM, about 0.01 mM to 10 mM, about 0.1 mM to 10 mM, about 1 μM to 10 μM, 10 μM to 100 μM, or about 100 μM to 1000 mM.

In some embodiment the epigenetic agent is administered systemically to a subject at a daily dose of about 0.01 mg to 1000 mg/day; about 0.01 mg to 500 mg/day; about 0.01 mg to 250 mg/day; about 0.01 mg to 100 mg/day; about 0.01 mg to 50 mg/day; about 0.01 mg to 25 mg/day; about 0.01 mg to 10 mg/day; about 0.01 mg to 5 mg/day; 0.1 mg to 100 mg/day; about 0.1 mg to 50 mg/day; about 0.01 mg to 25 mg/day; about 0.01 mg to 10 mg/day; about 0.01 mg to 5 mg/day; about 0.01 mg to 2.5 mg/day; about 0.1 mg to 10 mg/day; about 0.1 mg to 5 mg/day about 0.1 mg to 4 mg/day; about 0.1 mg to 3 mg/day; about 0.1 mg to 2 mg/day; about 0.1 mg to 2 mg/day or about 1 mg to 5 mg/day.

In some embodiments, the epigenetic agent is administered to the subject at a concentration ratio of about 0.001 to 10 fold relative to an FDA approved concentration or about 0.1 to 50 fold relative to an FDA approved concentration, or about 0.1 to 5 fold relative to an FDA approved, or about 1 to 5 fold relative to an FDA approved concentration. In some embodiments, the epigenetic agent is administered to the subject at about 0.01×. 0.1×, 2×, 3×, 5× or 10×, relative to an FDA approved concentration.

In some embodiments, the LSD1 inhibitor is GSK-2879552 and is administered for example to a cochlear cell in amount sufficient to achieve a concentration of about 0.001 nM to 1 mM, about 0.01 nM to 100 μM, about 0.1 nM to 10 μM, about 1 nM to 1 μM, about 1 nM to 10 nM, about 10 nM to 100 nM, about 100 nM to 1 μM, or about 1 μM to 10 μM in the perilymph fluid in the inner ear.

In some embodiments, the GSK-2879552 is administered, in amount sufficient to achieve a concentration of about 0.1 nM, 0.2 nM, 0.3 nM, 0.4 nM, 0.5 nM, 0.6 nM, 0.7 nM, 0.8 nM, 0.9 nM, 1.0 nM, 2.0 nM, 3.0 nM, 4.0 nM, 5.0 nM, 6.0 nM, 7.0 nM, 8.0 nM, 9.0 nM, 10 nM, 20 nM, 30 nM, 40 nM, 50 nM, 60 nM, 70 nM, 80 nM, 90 nM, 100 nM, 200 nM, 300 nM, 400 nM, 500 nM, 600 nM, 700 nM, 800 nM, 900 nM, 1 μM, 2 μM, 3 μM, 4 μM, 5 μM, 6 μM, 7 μM, 8 μM, 9 μM, 10 μM, 12 μM, 14 μM, 16 μM, 18 μM, 20 μM, 25 μM, or about 30 μM in the perilymph fluid in the inner ear.

In some embodiments, the LSD1 inhibitor is GSK-2879552 is administered to a subject, for example to the middle ear at a concentration of 0.001 μM to 1,000 mM, about 0.01 μM to 100,000 μM, about 0.1 μM to 10,000 μM, about 1 μM to 1,000 μM, about 1 μM to 10 μM, about 10 μM to 100 μM, about 100 μM to 1 mM, or about 1 mM to 10 mM.

In some embodiments, the GSK-2879552 is administered to a subject, for example to the middle ear at a concentration of about 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, 10 mM, 12 mM, 14 mM, 16 mM, 18 mM, 20 mM, 25 mM or about 30 mM.

In some embodiments, the LSD-1 inhibitor is GSK-2879552 and is administered to a subject systemically at a daily dose of about 0.01 mg to 500 mg/day about 0.1 mg to 100 mg/day, about 1 mg to 50 mg/day, about 1 mg to 25 mg/day, about 1 mg to 10 mg/day, about 1 mg to 5 mg/day, about 0.01 mg to 0.1 mg/day, about 0.1 mg to 1 mg/day, about 1 mg to 10 mg/day, about 10 mg to 100 mg/day, about 100 mg to 500 mg/day, about 0.5 mg to 1 mg/day, about 1 mg to 2 mg/day, about 2 mg to 3 mg/day, about 3 mg to 4 mg/day, about 4 mg to 5 mg/day, or about 5-10 mg/day.

In some embodiments, the LSD1 inhibitor is GSK-2879552 and is administered to the subject at a concentration ratio of about 0.001 to 100 fold relative to an FDA approved concentration or about 0.01 to 50 fold relative to an FDA approved concentration or about 0.1 to 10 fold relative to an FDA approved concentration, or about 0.1 to 5 fold relative to an FDA approved, or about 1 to 5 fold relative to an FDA approved concentration.

In some embodiments, LSD1 inhibitor is GSK-2879552 and is administered to the subject at about 0.01×. 0.1×, 1×, 2×, 3×, 4×, 5× or 10×, relative to an FDA approved concentration. A GSK-2879552 FDA approved concentration is for example the concentration listed on Table 1, column titled “Human Dosage”.

In some embodiments, the LSD1 inhibitor is GSK-LSD1 and is administered for example to a cochlear cell in amount sufficient to achieve a concentration of about 0.001 nM to 10 uM, about 0.01 nM to 1 uM, about 0.1 nM to 100 nM, about 0.001 nM to 0.01 nM, about 0.01 nM to 0.1 nM, about 0.1 nM to 1 nM, about 1 nM to 10 nM, about 10 nM to 100 nM, about 100 nM to 1,000 nM, 1 μM to 10 μM or about 10 μM to 100 μM in the perilymph fluid in the inner ear.

In some embodiments, the GSK-LSD1 is administered, in amount sufficient to achieve a concentration of about 0.1 nM, 0.2 nM, 0.3 nM, 0.4 nM, 0.5 nM, 0.6 nM, 0.7 nM, 0.8 nM, 0.9 nM, 1.0 nM, 2.0 nM, 3.0 nM, 4.0 nM, 5.0 nM, 6.0 nM, 7.0 nM, 8.0 nM, 9.0 nM, 10 nM, 2.0 nM, 30 nM, 40 nM, 50 nM, 60 nM, 70 nM, 80 nM, 90 nM, 100 nM, 200 nM, 300 nM, 400 nM, 500 nM, 1 μM, 5 μM, 10 μM, or 50 μM in the perilymph fluid in the inner ear.

In some embodiments, the LSD1 inhibitor is GSK-LSD1 is administered to a subject, for example to the middle ear at a concentration of 0.001 μM to 10 mM, about 0.01 μM to 1 mM, about 0.1 μM to 100 μM, about 0.001 μM to 0.01 μM, about 0.01 μM to 0.1 μM, about 0.1 μM to 1 μM, about 1 μM to 10 μM, about 10 μM to 100 μM, about 100 μM to 1,000 μM or about 1 mM to 50 mM.

In some embodiments, the GSK-LSD1 is administered to a subject, for example to the middle ear at a concentration of about 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 1 mM, 5 mM, 10 mM, or 50 mM.

In some embodiments, the LSD-1 inhibitor is GSK-LSD1 and is administered to a subject systemically at a daily dose of about 0.01 mg to 500 mg/day, about 0.1 mg to 100 mg/day, about 1 mg to 50 mg/day, about 1 mg to 25 mg/day, about 1 mg to 10 mg/day, about 1 mg to 5 mg/day, about 0.01 mg to 0.1 mg/day, about 0.1 mg to 1 mg/day, about 1 mg to 10 mg/day, about 10 mg to 100 mg/day, about 100 mg to 500 mg/day, about 0.5 mg to 1 mg/day, about 1 mg to 2 mg/day, about 2 mg to 3 mg/day, about 3 mg to 4 mg/day, about 4 mg to 5 mg/day, about 5-10 mg/day, about 10-25 mg/day, about 25-50 mg/day, or about 50-100 mg/day.

In some embodiments, the LSD1 inhibitor is GSK-LSD1 and is administered to the subject at a concentration ratio of about 0.001 to 100 fold relative to an FDA approved concentration or about 0.01 to 50 fold relative to an FDA approved concentration, or about 0.1 to 10 fold relative to an FDA approved concentration, or about 0.1 to 5 fold relative to an FDA approved concentration, or about 1 to 5 fold relative to an FDA approved concentration.

In some embodiments, LSD1 inhibitor is GSK-LSD1 and is administered to the subject at about 0.01×. 0.1×, 2×, 3×, 4×, 5× or 10×, relative to an FDA approved concentration. A GSK-LSD1 FDA approved concentration is for example the concentration listed on Table 1, column titled “Human Dosage”.

In some embodiments, the LSD-1 inhibitor is Tranylcypromine, and is administered for example to a cochlear cell in amount sufficient to achieve a concentration of about 0.001 μM to 10 mM, about 0.01 μM to 1 mM, about 0.1 μM to 100 μM, about 0.001 μM to 0.01 μM, about 0.01 μM to 0.1 μM, about 0.1 μM to 1 μM, about 1 μM to 10 μM, about 10 μM to 100 μM, about 100 μM to 1,000 μM or about 1 mM to 10 mM in the perilymph fluid in the inner ear.

In some embodiments, the Tranylcypromine is administered, for example to a cochlear cell in amount sufficient to achieve a concentration of about 0.1 μM, 1 μM, 2 μM, 3 μM, 4 μM, 5 μM, 6 μM, 7 μM, 8 μM, 9 μM, 10 μM, 12 μM, 14 μM, 16 μM, 18 μM or 20 μM in the perilymph fluid in the inner ear.

In some embodiments, the LSD-1 inhibitor is Tranylcypromine, and is administered to a subject, for example to the middle ear at a concentration of about 0.001 mM to 10,000 mM, about 0.01 mM to 1,000 mM, about 0.1 mM to 100 mM, about 0.001 mM to 0.01 mM, about 0.01 mM to 0.1 mM, about 0.1 mM to 1 mM, about 1 mM to 10 mM, about 10 mM to 100 mM, about 100 mM to 1,000 mM, or about 1,000 mM to 10,000 mM.

In some embodiments, the Tranylcypromine to a subject, for example to the middle ear at a concentration of about 0.1 mM, 0.2 mM, 0.3 mM, 0.4 mM, 0.5 mM, 0.6 mM, 0.7 mM, 0.8 mM, 0.9 mM, 1 mM, 2 mM, 3 mM, 4 mM, 5 mM, 6 mM, 7 mM, 8 mM, 9 mM, 10 mM, 12 mM, 14 mM, 16 mM, 18 mM, or 20 mM.

In some embodiments, the LSD-1 inhibitor is Tranylcypromine and is administered to a subject systemically at a daily dose of about 1.5 mg to 750 mg/day, about 5 mg to 500 mg/day, about 10 mg to 250 mg/day, about 15 mg to 150 mg/day, about 1.5 mg to 10 mg/day, about 10 mg to 20 mg/day, about 20 mg to 30 mg/day, about 30 mg to 40 mg/day, about 40 mg to 50 mg/day, about 50 mg to 60 mg/day, about 60 mg to 70 mg/day, about 70 mg to 80 mg/day, about 90 mg to 100 mg/day, about 100 mg to 120 mg/day, or about 120 mg to 150 mg/day.

In some embodiments, the LSD1 inhibitor is Tranylcypromine and is administered to the subject at a concentration ratio of about 0.001 to 100 fold relative to an FDA approved concentration or about 0.01 to 50 fold relative to an FDA approved concentration, or about 0.1 to 5 fold relative to an FDA approved, or about 1 to 5 fold relative to an FDA approved concentration.

In some embodiments, LSD1 inhibitor is Tranylcypromine and is administered to the subject at about 0.01×. 0.1×, 2×, 3×, 4×, 5× or 10×, relative to an FDA approved concentration. A Tranylcypromine FDA approved concentration is for example the concentration listed on Table 1, column titled “Human Dosage”.

In some embodiments, the LSD-1 inhibitor is Phenelzine sulfate, and is administered for example to a cochlear cell in amount sufficient to achieve a concentration of 0.001 μM to 100 mM, about 0.01 μM to 10 mM, about 0.1 μM to 1 mM, about 1 μM to 100 μM, about 0.001 μM to 0.01 μM, about 0.01 μM to 0.1 μM, about 0.1 μM to 1 μM, about 1 μM to 10 μM, about 10 μM to 100 μM, about 100 μM to 1,000 μM, or about 1 mM to 10 mM in the perilymph fluid in the inner ear.

In some embodiments, the Phenelzine sulfate is administered, for example to a cochlear cell in amount sufficient to achieve a concentration of about 0.1 μM, 0.2 μM, 0.3 μM, 0.4 uM, 0.5 μM, 0.6 μM, 0.7 μM, 0.8 μM, 0.9 μM, 1 μM, 2 μM, 3 μM, 4 μM, 5 μM, 6 μM, 7 μM, 8 μM, 9 μM, or 10 μM in the perilymph fluid in the inner ear.

In some embodiments, the LSD-1 inhibitor is Phenelzine sulfate, and is administered to a subject, for example to the middle ear at a concentration of about 0.001 mM to 100,000 mM, 0.01 mM to 10,000 mM, about 0.1 mM to 1,000 mM, about 0.1 mM to 100 mM, about 0.001 mM to 0.01 mM, about 0.01 mM to 0.1 mM, about 0.1 mM to 1 mM, about 1 mM to 10 mM, about 10 mM to 100 mM, about 100 mM to 1,000 mM, or about 1,000 mM to 10,000 mM.

In some embodiments, the Phenelzine sulfate is administered to a subject, for example to the middle ear at a concentration of about 0.1 mM, 0.2 mM, 0.3 mM, 0.4 mM, 0.5 mM, 0.6 mM, 0.7 mM, 0.8 mM, 0.9 mM, 1 mM, 2 mM, 3 mM, 4 mM, 5 mM, 6 mM, 7 mM, 8 mM, 9 mM, or 10 mM.

In some embodiments, the LSD-1 inhibitor is Phenelzine sulfate and is administered to a subject systemically at a daily dose of about 1.5 mg to 750 mg/day, about 5 mg to 500 mg/day, about 10 mg to 250 mg/day, about 15 mg to 150 mg/day, about 1.5 mg to 10 mg/day, about 10 mg to 20 mg/day, about 20 mg to 30 mg/day; about 30 mg to 40 mg/day; about 40 mg to 50 mg/day about 50 mg to 60 mg/day; about 60 mg to 70 mg/day; about 70 mg to 80 mg/day; or about 90 mg to 100 mg/day

In some embodiments, the LSD1 inhibitor is Phenelzine sulfate and is administered to the subject at a concentration ratio of about 0.001 to 100 fold relative to an FDA approved concentration or about 0.01 to 50 fold relative to an FDA approved concentration, or about 0.1 to 5 fold relative to an FDA approved, or about 1 to 5 fold relative to an FDA approved concentration.

In some embodiments, LSD1 inhibitor is Phenelzine sulfate and is administered to the subject at about 0.01×. 0.1×, 2×, 3×, 4×, 5× or 10×, relative to an FDA approved concentration. A Tranylcypromine FDA approved concentration is for example the concentration listed on Table 1, column titled “Human Dosage”.

In some embodiments, the LSD1 inhibitor is ORY-1001 and is administered for example to a cochlear cell in amount sufficient to achieve a concentration of about 0.001 nM to 1 mM, about 0.01 nM to 100 μM, about 0.1 nM to 10 μM, about 1 nM to 1 μM, about 1 nM to 10 nM, about 10 nM to 100 nM, about 100 nM to 1 μM, or about 1 μM to 10 μM in the perilymph fluid in the inner ear.

In some embodiments, the ORY-1001 is administered, in amount sufficient to achieve a concentration of about 0.1 nM, 0.2 nM, 0.3 nM, 0.4 nM, 0.5 nM, 0.6 nM, 0.7 nM, 0.8 nM, 0.9 nM, 1.0 nM, 2.0 nM, 3.0 nM, 4.0 nM, 5.0 nM, 6.0 nM, 7.0 nM, 8.0 nM, 9.0 nM, 10 nM, 20 nM, 30 nM, 40 nM, 50 nM, 60 nM, 70 nM, 80 nM, 90 nM, 100 nM, 200 nM, 300 nM, 400 nM, 500 nM, 600 nM, 700 nM, 800 nM, 900 nM, 1 μM, 2 μM, 3 μM, 4 μM, 5 μM, 6 μM, 7 μM, 8 μM, 9 μM, 10 μM, 12 μM, 14 μM, 16 μM, 18 μM, 20 μM, 25 μM, or about 30 μM in the perilymph fluid in the inner ear.

In some embodiments, the LSD1 inhibitor is ORY-1001 is administered to a subject, for example to the middle ear at a concentration of 0.001 μM to 1,000 mM, about 0.01 μM to 100,000 μM, about 0.1 μM to 10,000 μM, about 1 μM to 1,000 μM, about 1 μM to 10 μM, about 10 μM to 100 μM, about 100 μM to 1 mM, or about 1 mM to 10 mM.

In some embodiments, the ORY-1001 is administered to a subject, for example to the middle ear at a concentration of about 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, 10 mM, 12 mM, 14 mM, 16 mM, 18 mM, 20 mM, 25 mM or about 30 mM.

In some embodiments, the LSD-1 inhibitor is ORY-1001 and is administered to a subject systemically at a daily dose of about 0.01 mg to 500 mg/day about 0.1 mg to 100 mg/day, about 1 mg to 50 mg/day, about 1 mg to 25 mg/day, about 1 mg to 10 mg/day, about 1 mg to 5 mg/day, about 0.01 mg to 0.1 mg/day, about 0.1 mg to 1 mg/day, about 1 mg to 10 mg/day, about 10 mg to 100 mg/day, about 100 mg to 500 mg/day, about 0.5 mg to 1 mg/day, about 1 mg to 2 mg/day, about 2 mg to 3 mg/day, about 3 mg to 4 mg/day, about 4 mg to 5 mg/day, or about 5-10 mg/day.

In some embodiments, the LSD1 inhibitor is ORY-1001 and is administered to the subject at a concentration ratio of about 0.001 to 100 fold relative to an FDA approved concentration or about 0.01 to 50 fold relative to an FDA approved concentration or about 0.1 to 10 fold relative to an FDA approved concentration, or about 0.1 to 5 fold relative to an FDA approved, or about 1 to 5 fold relative to an FDA approved concentration.

In some embodiments, LSD1 inhibitor is ORY-1001 and is administered to the subject at about 0.01×. 0.1×, 1×, 2×, 3×, 4×, 5× or 10×, relative to an FDA approved concentration. A ORY-1001 FDA approved concentration is for example the concentration listed on Table 1, column titled “Human Dosage”.

In some embodiments, the LSD1 inhibitor is RN-1 and is administered for example to a cochlear cell in amount sufficient to achieve a concentration of about 0.001 nM to 1 mM, about 0.01 nM to 100 μM, about 0.1 nM to 10 μM, about 1 nM to 1 μM, about 1 nM to 10 nM, about 10 nM to 100 nM, about 100 nM to 1 μM, or about 1 μM to 10 μM in the perilymph fluid in the inner ear.

In some embodiments, the RN-1 is administered, in amount sufficient to achieve a concentration of about 0.1 nM, 0.2 nM, 0.3 nM, 0.4 nM, 0.5 nM, 0.6 nM, 0.7 nM, 0.8 nM, 0.9 nM, 1.0 nM, 2.0 nM, 3.0 nM, 4.0 nM, 5.0 nM, 6.0 nM, 7.0 nM, 8.0 nM, 9.0 nM, 10 nM, 20 nM, 30 nM, 40 nM, 50 nM, 60 nM, 70 nM, 80 nM, 90 nM, 100 nM, 200 nM, 300 nM, 400 nM, 500 nM, 600 nM, 700 nM, 800 nM, 900 nM, 1 μM, 2 μM, 3 μM, 4 μM, 5 μM, 6 μM, 7 μM, 8 μM, 9 μM, 10 μM, 12 μM, 14 μM, 16 μM, 18 μM, 20 μM, 25 μM, or about 30 μM in the perilymph fluid in the inner ear.

In some embodiments, the LSD1 inhibitor is RN-1 is administered to a subject, for example to the middle ear at a concentration of 0.001 μM to 1,000 mM, about 0.01 μM to 100,000 μM, about 0.1 μM to 10,000 μM, about 1 μM to 1,000 μM, about 1 μM to 10 μM, about 10 μM to 100 μM, about 100 μM to 1 mM, or about 1 mM to 10 mM.

In some embodiments, the RN-1 is administered to a subject, for example to the middle ear at a concentration of about 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, 10 mM, 12 mM, 14 mM, 16 mM, 18 mM, 20 mM, 25 mM or about 30 mM.

In some embodiments, the LSD1 inhibitor is RN-1 and is administered to a subject systemically at a daily dose of about 0.01 mg to 500 mg/day about 0.1 mg to 100 mg/day, about 1 mg to 50 mg/day, about 1 mg to 25 mg/day, about 1 mg to 10 mg/day, about 1 mg to 5 mg/day, about 0.01 mg to 0.1 mg/day, about 0.1 mg to 1 mg/day, about 1 mg to 10 mg/day, about 10 mg to 100 mg/day, about 100 mg to 500 mg/day, about 0.5 mg to 1 mg/day, about 1 mg to 2 mg/day, about 2 mg to 3 mg/day, about 3 mg to 4 mg/day, about 4 mg to 5 mg/day, or about 5-10 mg/day.

In some embodiments, the LSD1 inhibitor is RN-1 and is administered to the subject at a concentration ratio of about 0.001 to 100 fold relative to an FDA approved concentration or about 0.01 to 50 fold relative to an FDA approved concentration or about 0.1 to 10 fold relative to an FDA approved concentration, or about 0.1 to 5 fold relative to an FDA approved, or about 1 to 5 fold relative to an FDA approved concentration.

In some embodiments, LSD1 inhibitor is RN-1 and is administered to the subject at about 0.01×. 0.1×, 1×, 2×, 3×, 4×, 5× or 10×, relative to an FDA approved concentration. A GSK-2879552 FDA approved concentration is for example the concentration listed on Table 1, column titled “Human Dosage”.

In some embodiments, the GSK3 Inhibitor is AZD1080, and is administered for example to a cochlear cell in amount sufficient to achieve a concentration of about 0.001 μM to 10 mM, about 0.01 uM to 1 mM, about 0.1 μM to 100 μM, about 0.001 μM to 0.01 μM, about 0.01 μM to 0.1 μM, about 0.1 μM to 1 μM, about 1 μM to 10 μM, about 10 μM to 100 μM, about 100 μM to 1,000 μM, or about 1 mM to 10 mM in the perilymph fluid in the inner ear.

In some embodiments, the AZD1080 is administered, is administered, in amount sufficient to achieve a concentration of about is about 1 μM, 2 μM, 3 μM, 4 μM, 5 μM, 6 μM, 7 μM, 8 μM, 9 μM, or 10 μM, in the perilymph fluid in the inner ear.

In some embodiments, the GSK3 Inhibitor is AZD1080, and is administered to a subject, for example to the middle ear at a concentration of about 0.001 mM to 10,000 mM, about 0.01 mM to 1,000 mM, about 0.1 mM to 100 mM, about 0.001 mM to 0.01 mM, about 0.01 mM to 0.1 mM, about 0.1 mM to 1 mM, about 1 mM to 10 mM, about 10 mM to 100 mM, about 100 mM to 1,000 mM, or about 1,000 mM to 10,000 mM.

In some embodiments, the AZD1080 is administered to a subject, for example to the middle ear at a concentration of about 1 mM, 2 mM, 3 mM, 4 mM, 5 mM, 6 mM, 7 mM, 8 mM, 9 mM, or 10 mM.

In some embodiments, the GSK3 Inhibitor is AZD1080 and is administered to the subject at a concentration ratio of about 0.001 to 10 fold relative to an FDA approved concentration or about 0.1 to 50 fold relative to an FDA approved concentration, or about 0.1 to 5 fold relative to an FDA approved, or about 1 to 5 fold relative to an FDA approved concentration.

In some embodiments, the GSK3 Inhibitor is AZD1080 and is administered to the subject at about 0.01×. 0.1×, 2×, 3×, 5× or 10×, relative to an FDA approved concentration

In some embodiments, the GSK3 Inhibitor is LY2090314, and is administered for example to a cochlear cell in amount sufficient to achieve a concentration of about 0.001 nM to 10 mM, about 0.01 nM to 1 μM, about 0.1 nM to 100 nM, about 0.001 nM to 0.01 nM, about 0.01 nM to 0.1 nM, about 0.1 nM to 1 nM, about 1 nM to 10 nM, about 10 nM to 100 nM, about 100 nM to 1 μM, or about 1 μM to 10 μM, in the perilymph fluid in the inner ear.

In some embodiments, the LY2090314 is administered, in amount sufficient to achieve a concentration of about 1 nM, 5 nM, 10 nM, 15 nM, 20 nM, or 40 nM, in the perilymph fluid in the inner ear.

In some embodiments, the GSK3 Inhibitor is LY2090314, and is administered to a subject, for example to the middle ear at a concentration of about 0.001 μM to 10 mM, about 0.01 μM to 1 mM, about 0.1 μM to 100 uM, about 0.001 μM to 0.01 μM, about 0.01 μM to 0.1 μM, about 0.1 μM to 1 μM, about 1 μM to 10 μM, about 10 μM to 100 μM, about 100 μM to 1 mM, or about 1 mM to 10 mM.

In some embodiments, LY2090314 the is administered to a subject, for example to the middle ear at a concentration of about 1 μM, 5 μM, 10 μM, 15 μM, 20 μM, or 40 μM.

In some embodiments, the GSK3 Inhibitor is LY2090314 and is administered to the subject at a concentration ratio of about 0.001 to 10 fold relative to an FDA approved concentration or about 0.1 to 50 fold relative to an FDA approved concentration, or about 0.1 to 5 fold relative to an FDA approved, or about 1 to 5 fold relative to an FDA approved concentration.

In some embodiments, the GSK3 Inhibitor is LY2090314 and is administered to the subject at about 0.01×. 0.1×, 2×, 3×, 5× or 10×, relative to an FDA approved concentration.

In some embodiments, the GSK3 Inhibitor is a substituted 3-Imidazo[1,2-a]pyridin-3-yl-4-(1,2,3,4-tetrahydro-[1,4]diazepino-[6,7,1-hi]indol-7-yl)pyrrole-2,5-dione, and is administered for example to a cochlear cell in amount sufficient to achieve a concentration of about 0.001 nM to 10 mM, about 0.01 nM to 1 μM, about 0.1 nM to 100 nM, about 0.001 nM to 0.01 nM, about 0.01 nM to 0.1 nM, about 0.1 nM to 1 nM, about 1 nM to 10 nM, about 10 nM to 100 nM, about 100 nM to 1 μM, or about 1 μM to 10 μM, in the perilymph fluid in the inner ear.

In some embodiments, the substituted 3-Imidazo[1,2-a]pyridin-3-yl-4-(1,2,3,4-tetrahydro-[1,4]diazepino-[6,7,1-hi]indol-7-yl)pyrrole-2,5-dione, is administered, in amount sufficient to achieve a concentration of about 1 nM, 5 nM, 10 nM, 15 nM, 20 nM, 50 nM, 100 nM, 250 nM, or 500 nM, in the perilymph fluid in the inner ear.

In some embodiments, the GSK3 Inhibitor is a substituted 3-Imidazo[1,2-a]pyridin-3-yl-4-(1,2,3,4-tetrahydro-[1,4]diazepino-[6,7,1-hi]indol-7-yl)pyrrole-2,5-dione, and is administered to a subject, for example to the middle ear at a concentration of about 0.001 μM to 10 mM, about 0.01 μM to 1 mM, about 0.1 μM to 100 uM, about 0.001 μM to 0.01 μM, about 0.01 μM to 0.1 μM, about 0.1 μM to 1 μM, about 1 μM to 10 μM, about 10 μM to 100 μM, about 100 μM to 1 mM, or about 1 mM to 10 mM.

In some embodiments, the substituted 3-Imidazo[1,2-a]pyridin-3-yl-4-(1,2,3,4-tetrahydro-[1,4]diazepino-[6,7,1-hi]indol-7-yl)pyrrole-2,5-dione, the is administered to a subject, for example to the middle ear at a concentration of about 1 μM, 5 μM, 10 μM, 15 μM, 20 μM, 50 μM, 100 μM, 250 μM, or 500 μM.

In some embodiments, the GSK3 Inhibitor is a substituted 3-Imidazo[1,2-a]pyridin-3-yl-4-(1,2,3,4-tetrahydro-[1,4]diazepino-[6,7,1-hi]indol-7-yl)pyrrole-2,5-dione, and is administered to the subject at a concentration ratio of about 0.001 to 10 fold relative to an FDA approved concentration or about 0.1 to 50 fold relative to an FDA approved concentration, or about 0.1 to 5 fold relative to an FDA approved, or about 1 to 5 fold relative to an FDA approved concentration.

In some embodiments, the GSK3 Inhibitor is a substituted 3-Imidazo[1,2-a]pyridin-3-yl-4-(1,2,3,4-tetrahydro-[1,4]diazepino-[6,7,1-hi]indol-7-yl)pyrrole-2,5-dione and is administered to the subject at about 0.01×. 0.1×, 2×, 3×, 5× or 10×, relative to an FDA approved concentration

In some embodiments, the GSK3 inhibitor is GSK3-inhibitor XXII, and is administered for example to a cochlear cell in amount sufficient to achieve a concentration of about 0.1 nM to 1 mM, about 1 nM to 100 μM, about 10 nM to 10 μM, about 0.1 nM to 1 nM, about 1 nM to 10 nM, about 10 nM to 100 nM, about 100 nM to 1 μM, about 1 μM to 10 μM, about 10 μM to 100 μM, or about 100 μM to 1000 μM, in the perilymph fluid in the inner ear.

In some embodiments, the GSK3-inhibitor XXII is administered, in amount sufficient to achieve a concentration of about 0.1 μM, 0.2 μM, 0.3 μM, 0.4 μM, 0.5 μM, 0.6 μM, 0.7 μM, 0.8 μM, 0.9 μM, or 1.0 μM, in the perilymph fluid in the inner ear.

In some embodiments, the GSK3 Inhibitor is GSK3-inhibitor XXII, is administered to a subject, for example to the middle ear at a concentration of about of about 0.1 μM to 1,000 mM, about 1 μM to 100 mM, about 10 μM to 10 mM, about 0.1 μM to 1 μM, about 1 μM to 10 μM, about 10 μM to 100 μM, about 100 μM to 1 mM, about 1 mM to 10 mM, about 10 mM to 100 mM, or about 100 mM to 1000 mM. In some embodiments, the GSK3-inhibitor XXII is administered, to a subject, for example to the middle ear at a concentration of about 0.1 mM, 0.2 mM, 0.3 mM, 0.4 mM, 0.5 mM, 0.6 mM, 0.7 mM, 0.8 mM, 0.9 mM, or 1.0 mM

In some embodiments, the GSK3 Inhibitor is GSK3-inhibitor XXII and is administered to the subject at a concentration ratio of about 0.001 to 10 fold relative to an FDA approved concentration or about 0.1 to 50 fold relative to an FDA approved concentration, or about 0.1 to 5 fold relative to an FDA approved, or about 1 to 5 fold relative to an FDA approved concentration.

In some embodiments, the GSK3 Inhibitor is GSK3-inhibitor XXII and is administered to the subject at about 0.01×. 0.1×, 2×, 3×, 5× or 10×, relative to an FDA approved concentration.

In some embodiments, the GSK3 Inhibitor is CHIR99021, and is administered for example to a cochlear cell in amount sufficient to achieve a concentration of about 0.001 mM to 10 mM, about 0.01 mM to 1 mM, about 0.1 μM to 100 μM, about 0.001 μM to 0.01 μM, about 0.01 μM to 0.1 μM, about 0.1 μM to 1 μM, about 1 μM to 10 μM, about 10 μM to 100 μM, about 100 μM to 1,000 μM, or about 1 mM to 10 mM, in the perilymph fluid in the inner ear.

In some embodiments, the CHIR99021 is administered, in amount sufficient to achieve a concentration of about 1 μM, 2 μM, 3 μM, 4 μM, 5 μM, 6 μM, 7 μM, 8 μM, 9 μM, or 10 μM, in the perilymph fluid in the inner ear.

In some embodiments, the GSK3 Inhibitor is CHIR99021, is administered to a subject, for example to the middle ear at a concentration of about 0.001 mM to 10,000 mM, about 0.01 mM to 1,000 mM, about 0.1 mM to 100 mM, about 0.001 mM to 0.01 mM, about 0.01 mM to 0.1 mM, about 0.1 mM to 1 mM, about 1 mM to 10 mM, about 10 mM to 100 mM, about 100 mM to 1,000 mM, or about 1,000 mM to 10,000 mM.

In some embodiments, the CHIR99021 is administered to a subject, for example to the middle ear at a concentration of about 1 mM, 2 mM, 3 mM, 4 mM, 5 mM, 6 mM, 7 mM, 8 mM, 9 mM, or 10 mM.

In some embodiments, the GSK3 Inhibitor is CHIR99021 and is administered to the subject at a concentration ratio of about 0.001 to 10 fold relative to an FDA approved concentration or about 0.1 to 50 fold relative to an FDA approved concentration, or about 0.1 to 5 fold relative to an FDA approved, or about 1 to 5 fold relative to an FDA approved concentration.

In some embodiments, the GSK3 Inhibitor is CHIR99021 and is administered to the subject at about 0.01×. 0.1×, 2×, 3×, 5× or 10×, relative to an FDA approved concentration.

In various embodiments, the methods further comprise administering one more additional epigenetic agents, such as an HDAC inhibitor, an EZH2 inhibitor, a DOT1L inhibitor, or a KDM inhibitor as described herein.

In some embodiments the additional epigenetic agent is an HDAC inhibitor and is administered for example to a cochlear cell in amount sufficient to achieve a concentration of about is about 0.01 uM to 1000 mM, about 1 uM to 100 mM, about 10 uM to 10 mM, about 1 uM to 10 uM, about 10 uM to 100 uM, about 100 uM to 1000 uM, about 1 mM to 10 mM, or about 10 mM to 100 mM in the perilymph fluid in the inner ear.

In some embodiments the HDAC inhibitor is administered, to a subject, for example to the middle ear at a concentration about 10 uM to 1,000,000 mM, about 1000 uM to 100,000 mM, about 10,000 uM to 10,000 mM, about 1000 uM to 10,000 uM, about 10,000 uM to 100,000 uM, about 100,000 uM to 1,000,000 uM, about 1,000 mM to 10,000 mM, or about 10,000 mM to 100,000 mM.

In some embodiments, the HDAC inhibitor is VPA and is administered for example to a cochlear cell in amount sufficient to achieve a concentration of about is about 10 μM to 4 mM in the perilymph fluid in the inner ear.

In some embodiments VPA is administered, to a subject, for example to the middle ear at a concentration about 100 mM to 4,000 mM.

In some embodiments, the HDAC inhibitor is VPA and is administered to a subject systemically at a daily dose of about 50 mg, about 100 mg, about 125 mg, about 250 mg, about 500 mg, 1000 mg, 2000 mg, 3000 mg, 4000 mg, or about 5000 mg In some embodiments, the VPA is administered as an oral dosage form of about 50 mg, about 100 mg, about 125 mg, about 250 mg, about 500 mg, 1000 mg, 2000 mg, 3000 mg, 4000 mg, or about 5000 mg

In some embodiments, the HDAC inhibitor is 2-hexyl-4pentynoic acid and is administered for example to a cochlear cell in amount sufficient to achieve a concentration of about is about 10 μM to 4 mM in the perilymph fluid in the inner ear.

In some embodiments 2-hexyl-4-pentynoic acid is administered, to a subject, for example to the middle ear at a concentration about 100 mM to 4,000 mM.

In some embodiments, the HDAC inhibitor is 2-hexyl-4-pentynoic acid and is administered to a subject systemically at a daily dose of about 50 mg, about 100 mg, about 125 mg, about 250 mg, about 500 mg, 1000 mg, 2000 mg, 3000 mg, 4000 mg, or about 5000 mg In some embodiments, the VPA is administered as an oral dosage form of about 50 mg, about 100 mg, about 125 mg, about 250 mg, about 500 mg, 1000 mg, 2000 mg, 3000 mg, 4000 mg, or about 5000 mg

In some embodiments, the HDAC inhibitor is Na phenylbutyrate and is administered for example to a cochlear cell in amount sufficient to achieve a concentration of about is about 10 μM to 4 mM in the perilymph fluid in the inner ear.

In some embodiments Na phenylbutyrate is administered, to a subject, for example to the middle ear at a concentration about 100 mM to 4,000 mM.

In some embodiments, the HDAC inhibitor is Na phenylbutyrate and is administered to a subject systemically at a daily dose of about 50 mg, about 100 mg, about 125 mg, about 250 mg, about 500 mg, 1000 mg, 2000 mg, 3000 mg, 4000 mg, or about 5000 mg In some embodiments, the VPA is administered as an oral dosage form of about 50 mg, about 100 mg, about 125 mg, about 250 mg, about 500 mg, 1000 mg, 2000 mg, 3000 mg, 4000 mg, or about 5000 mg

In some embodiments the LSD-1 inhibitor is GSK-2879552 and the Wnt agonist is LY2090314. In some embodiments, GSK-2879552 is administered, in amount sufficient to achieve a concentration of about 0.1 nM, 0.2 nM, 0.3 nM, 0.4 nM, 0.5 nM, 0.6 nM, 0.7 nM, 0.8 nM, 0.9 nM, 1.0 nM, 2.0 nM, 3.0 nM, 4.0 nM, 5.0 nM, 6.0 nM, 7.0 nM, 8.0 nM, 9.0 nM, 10 nM, 20 nM, 30 nM, 40 nM, 50 nM, 60 nM, 70 nM, 80 nM, 90 nM, 100 nM, 200 nM, 300 nM, 400 nM, 500 nM, 600 nM, 700 nM, 800 nM, 900 nM, 1 μM, 2 μM, 3 μM, 4 μM, 5 μM, 6 μM, 7 μM, 8 μM, 9 μM, 10 μM, 12 μM, 14 μM, 16 μM, 18 μM, 20 μM, 25 μM, of about 30 μM in the perilymph fluid in the inner ear and LY2090314 is administered, in amount sufficient to achieve a concentration of about 1 nM, 5 nM, 10 nM, 15 nM, 20 nM, or 40 nM in the perilymph fluid in the inner ear. Alternatively, the GSK-2879552 is administered to a subject, for example to the middle ear at a concentration of about 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, 10 mM, 12 mM, 14 mM, 16 mM, 18 mM, 20 mM, 25 mM or about 30 mM and LY2090314, and is administered to a subject, for example to the middle ear at a concentration of about 1 μM, 5 μM, 10 μM, 15 μM, 20 μM, or 40 μM.

In some embodiments the LSD-1 inhibitor is GSK-2879552 and the Wnt agonist is a substituted 3-Imidazo[1,2-a]pyridin-3-yl-4-(1,2,3,4-tetrahydro-[1,4]diazepino-[6,7,1-hi]indol-7-yl)pyrrole-2,5-dione. In some embodiments, GSK-2879552 is administered, in amount sufficient to achieve a concentration of about 0.1 nM, 0.2 nM, 0.3 nM, 0.4 nM, 0.5 nM, 0.6 nM, 0.7 nM, 0.8 nM, 0.9 nM, 1.0 nM, 2.0 nM, 3.0 nM, 4.0 nM, 5.0 nM, 6.0 nM, 7.0 nM, 8.0 nM, 9.0 nM, 10 nM, 20 nM, 30 nM, 40 nM, 50 nM, 60 nM, 70 nM, 80 nM, 90 nM, 100 nM, 200 nM, 300 nM, 400 nM, 500 nM, 600 nM, 700 nM, 800 nM, 900 nM, 1 μM, 2 μM, 3 μM, 4 μM, 5 μM, 6 μM, 7 μM, 8 μM, 9 μM, 10 μM, 12 μM, 14 μM, 16 μM, 18 μM, 20 μM, 25 μM, or about 30 μM in the perilymph fluid in the inner ear and the substituted 3-Imidazo[1,2-a]pyridin-3-yl-4-(1,2,3,4-tetrahydro-[1,4]diazepino-[6,7,1-hi]indol-7-yl)pyrrole-2,5-dione is administered, in amount sufficient to achieve a concentration of about 1 nM, 5 nM, 10 nM, 15 nM, 20 nM, 50 nM, 100 nM, 250 nM, or 500 nM, in the perilymph fluid in the inner ear. Alternatively, the GSK-2879552 is administered to a subject, for example to the middle ear at a concentration of about 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, 10 mM, 12 mM, 14 mM, 16 mM, 18 mM, 20 mM, 25 mM or about 30 mM and the substituted 3-Imidazo[1,2-a]pyridin-3-yl-4-(1,2,3,4-tetrahydro-[1,4]diazepino-[6,7,1-hi]indol-7-yl)pyrrole-2,5-dione and is administered to a subject, for example to the middle ear at a concentration of about 1 μM, 5 μM, 10 μM, 15 μM, 20 μM, 50 μM, 100 μM, 250 μM, or 500 μM.

In some embodiments the LSD-1 inhibitor is GSK-2879552 and the Wnt agonist is GSK3 inhibitor XXII. In some embodiments, GSK-2879552 is administered, in amount sufficient to achieve a concentration of about 0.1 nM, 0.2 nM, 0.3 nM, 0.4 nM, 0.5 nM, 0.6 nM, 0.7 nM, 0.8 nM, 0.9 nM, 1.0 nM, 2.0 nM, 3.0 nM, 4.0 nM, 5.0 nM, 6.0 nM, 7.0 nM, 8.0 nM, 9.0 nM, 10 nM, 20 nM, 30 nM, 40 nM, 50 nM, 60 nM, 70 nM, 80 nM, 90 nM, 100 nM, 200 nM, 300 nM, 400 nM, 500 nM, 600 nM, 700 nM, 800 nM, 900 nM, 1 μM, 2 μM, 3 μM, 4 μM, 5 μM, 6 μM, 7 μM, 8 μM, 9 μM, 10 μM, 12 μM, 14 μM, 16 μM, 18 μM, 20 μM, 25 μM, or about 30 μM in the perilymph fluid in the inner ear and GSK3-inhibitor XXII is administered, in amount sufficient to achieve a concentration of about 0.1 μM, 0.2 μM, 0.3 μM, 0.4 μM, 0.5 μM, 0.6 μM, 0.7 μM, 0.8 μM, 0.9 μM, or 1.0 μM, in the perilymph fluid in the inner ear. Alternatively, the GSK-2879552 is administered to a subject, for example to the middle ear at a concentration of about 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, 10 mM, 12 mM, 14 mM, 16 mM, 18 mM, 20 mM, 25 mM or about 30 mM and the GSK3-inhibitor XXII is administered, in amount sufficient to achieve a concentration of about 0.1 mM, 0.2 mM, 0.3 mM, 0.4 mM, 0.5 mM, 0.6 mM, 0.7 mM, 0.8 mM, 0.9 mM, or 1.0 mM, in the perilymph fluid in the inner ear.

In some embodiments the LSD-1 inhibitor is GSK-2879552 and the Wnt agonist is CHIR99021. In some embodiments, GSK-2879552 is administered, in amount sufficient to achieve a concentration of about 0.1 nM, 0.2 nM, 0.3 nM, 0.4 nM, 0.5 nM, 0.6 nM, 0.7 nM, 0.8 nM, 0.9 nM, 1.0 nM, 2.0 nM, 3.0 nM, 4.0 nM, 5.0 nM, 6.0 nM, 7.0 nM, 8.0 nM, 9.0 nM, 10 nM, 20 nM, 30 nM, 40 nM, 50 nM, 60 nM, 70 nM, 80 nM, 90 nM, 100 nM, 200 nM, 300 nM, 400 nM, 500 nM, 600 nM, 700 nM, 800 nM, 900 nM, 1 μM, 2 μM, 3 μM, 4 μM, 5 μM, 6 μM, 7 μM, 8 μM, 9 μM, 10 μM, 12 μM, 14 μM, 16 μM, 18 μM, 20 μM, 25 μM, or about 30 μM in the perilymph fluid in the inner ear and CHIR99021 is administered, in amount sufficient to achieve a concentration of about 1 μM, 2 μM, 3 μM, 4 μM, 5 μM, 6 μM, 7 μM, 8 μM, 9 μM, or 10 μM, in the perilymph fluid in the inner ear Alternatively, the GSK-2879552 is administered to a subject, for example to the middle ear at a concentration of about 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, 10 mM, 12 mM, 14 mM, 16 mM, 18 mM, 20 mM, 25 mM, or about 30 mM and CHIR99021 is administered to a subject, for example to the middle ear at a concentration of about 1 mM, 2 mM, 3 mM, 4 mM, 5 mM, 6 mM, 7 mM, 8 mM, 9 mM, or 10 mM.

In some embodiments the LSD-1 inhibitor is GSK-LSD1 and the Wnt agonist is AZD1080. In some embodiments, GSK-LSD1 is administered, in amount sufficient to achieve a concentration of about 0.1 nM, 0.2 nM, 0.3 nM, 0.4 nM, 0.5 nM, 0.6 nM, 0.7 nM, 0.8 nM, 0.9 nM, 1.0 nM, 2.0 nM, 3.0 nM, 4.0 nM, 5.0 nM, 6.0 nM, 7.0 nM, 8.0 nM, 9.0 nM, 10 nM, 20 nM, 30 nM, 40 nM, 50 nM, 60 nM, 70 nM, 80 nM, 90 nM, 100 nM, 200 nM, 300 nM, 400 nM, 500 nM, 1 μM, 5 μM, 10 μM, or 50 μM in the perilymph fluid in the inner ear and AZD1080 is administered, in amount sufficient to achieve a concentration of about is about 1 μM, 2 μM, 3 μM, 4 μM, 5 μM, 6 μM, 7 μM, 8 μM, 9 μM, or 10 μM, in the perilymph fluid in the inner ear. Alternatively, the GSK-LSD1 is administered to a subject, for example to the middle ear at a concentration of about 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 1 mM, 5 mM, 10 mM, or 50 μM and AZD1080, and is administered to a subject, for example to the middle ear at a concentration of about 1 mM, 2 mM, 3 mM, 4 mM, 5 mM, 6 mM, 7 mM, 8 mM, 9 mM, or 10 mM.

In some embodiments the LSD-1 inhibitor is GSK-LSD1 and the Wnt agonist is LY2090314 In some embodiments, GSK-LSD1 is administered, in amount sufficient to achieve a concentration of about 0.1 nM, 0.2 nM, 0.3 nM, 0.4 nM, 0.5 nM, 0.6 nM, 0.7 nM, 0.8 nM, 0.9 nM, 1.0 nM, 2.0 nM, 3.0 nM, 4.0 nM, 5.0 nM, 6.0 nM, 7.0 nM, 8.0 nM, 9.0 nM, 10 nM, 20 nM, 30 nM, 40 nM, 50 nM, 60 nM, 70 nM, 80 nM, 90 nM, 100 nM, 200 nM, 300 nM, 400 nM, 500 nM, 1 μM, 5 μM, 10 μM, or 50 μM in the perilymph fluid in the inner ear and LY2090314 is administered, in amount sufficient to achieve a concentration of about 1 nM, 5 nM, 10 nM, 15 nM, 20 nM or 40 nM, in the perilymph fluid in the inner ear. Alternatively, the GSK.-LSD1 is administered to a subject, for example to the middle ear at a concentration of about 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 1 mM, 5 mM, 10 mM, or 50 mM and LY2090314, and is administered to a subject, for example to the middle ear at a concentration of about 1 μM, 5 μM, 10 μM, 15 μM, 20 μM, or 40 μM.

In some embodiments the LSD-1 inhibitor is GSK-LSD1 and the Wnt agonist is a substituted 3-imidazo[1,2-a]pyridin-3-yl-4-(1,2,3,4-tetrahydro-[1,4]diazepino-[6,7,1-hi]indol-7-yl)pyrrole-2,5-dione. In some embodiments, GSK-LSD1 is administered, in amount sufficient to achieve a concentration of about 0.1 nM, 0.2 nM, 0.3 nM, 0.4 nM, 0.5 nM, 0.6 nM, 0.7 nM, 0.8 nM, 0.9 nM, 1.0 nM, 2.0 nM, 3.0 nM, 4.0 nM, 5.0 nM, 6.0 nM, 7.0 nM, 8.0 nM, 9.0 nM, 10 nM, 20 nM, 30 nM, 40 nM, 50 nM, 60 nM, 70 nM, 80 nM, 90 nM, 100 nM, 200 nM, 300 nM, 400 nM, 500 nM, 1 μM, 5 μM, 10 μM, or 50 μM in the perilymph fluid in the inner ear and the substituted 3-Imidazo[1,2-a]pyridin-3-yl-4-(1,2,3,4-tetrahydro-[1,4]diazepino-[6,7,1-hi]indol-7-yl)pyrrole-2,5-dione is administered, in amount sufficient to achieve a concentration of about 1 nM, 5 nM, 10 nM, 15 nM, 20 nM 50 nM, 100 nM, 250 nM, or 500 nM, in the perilymph fluid in the inner ear. Alternatively, the GSK-LSD1 is administered to a subject, for example to the middle ear at a concentration of about 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 1 mM, 5 mM, 10 mM, or 50 mM and the substituted 3-Imidazo[1,2-a]pyridin-3-yl-4-(1,2,3,4-tetrahydro-[1,4]diazepino-[6,7,1-hi]indol-7-yl)pyrrole-2,5-dione and is administered to a subject, for example to the middle ear at a concentration of about 1 μM, 5 μM, 10 μM, 15 μM, 20 μM, 50 μM, 100 μM, 250 μM, or 500 μM.

In some embodiments the LSD-1 inhibitor is GSK-LSD1 and the Wnt agonist is GSK3 inhibitor XXII. In some embodiments, GSK-LSD1 is administered, in amount sufficient to achieve a concentration of about 0.1 nM, 0.2 nM, 0.3 nM, 0.4 nM, 0.5 nM, 0.6 nM, 0.7 nM, 0.8 nM, 0.9 nM, 1.0 nM, 2.0 nM, 3.0 nM, 4.0 nM, 5.0 nM, 6.0 nM, 7.0 nM, 8.0 nM, 9.0 nM, 10 nM, 20 nM, 30 nM, 40 nM, 50 nM, 60 nM, 70 nM, 80 nM, 90 nM, 100 nM, 200 nM, 300 nM, 400 nM, 500 nM, 1 μM, 5 μM, 10 μM, or 50 μM in the perilymph fluid in the inner ear and GSK3-inhibitor XXII is administered, in amount sufficient to achieve a concentration of about 0.1 μM, 0.2 μM, 0.3 μM, 0.4 μM, 0.5 μM, 0.6 μM, 0.7 μM, 0.8 μM, 0.9 μM, or 1.0 μM, in the perilymph fluid in the inner ear. Alternatively, the GSK-LSD1 is administered to a subject, for example to the middle ear at a concentration of about 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 1 mM, 5 mM, 10 mM, or 50 mM, the GSK3-inhibitor XXII is administered, in amount sufficient to achieve a concentration of about 0.1 mM, 0.2 mM, 0.3 mM, 0.4 mM, 0.5 mM, 0.6 mM, 0.7 mM, 0.8 mM, 0.9 mM, or 1.0 mM, in the perilymph fluid in the inner ear.

In some embodiments the LSD-1 inhibitor is GSK-LSD1 and the Wnt agonist is CHIR99021. In some embodiments, GSK-LSD1 is administered, in amount sufficient to achieve a concentration of about 0.1 nM, 0.2 nM, 0.3 nM, 0.4 nM, 0.5 nM, 0.6 nM, 0.7 nM, 0.8 nM, 0.9 nM, 1.0 nM, 2.0 nM, 3.0 nM, 4.0 nM, 5.0 nM, 6.0 nM, 7.0 nM, 8.0 nM, 9.0 nM, 10 nM, 20 nM, 30 nM, 40 nM, 50 nM, 60 nM, 70 nM, 80 nM, 90 nM, 100 nM, 200 nM, 300 nM, 400 nM, 500 nM, 1 μM, 5 μM, 10 μM, or 50 μM in the perilymph fluid in the inner ear and CHIR99021 is administered, in amount sufficient to achieve a concentration of about 1 μM, 2 μM, 3 μM, 4 μM, 5 μM, 6 μM, 7 μM, 8 μM, 9 μM, or 10 μM, in the perilymph fluid in the inner ear Alternatively, the GSK-LSD1 is administered to a subject, for example to the middle ear at a concentration of about 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 1 mM, 5 mM, 10 mM, or 50 mM and CHIR99021 is administered to a subject, for example to the middle ear at a concentration of about 1 mM, 2 mM, 3 mM, 4 mM, 5 mM, 6 mM, 7 mM, 8 mM, 9 mM, or 10 mM.

In some embodiments the LSD-1 inhibitor is Tranylcypromine and the Wnt agonist is AZD1080. In some embodiments, Tranylcypromine is administered, in amount sufficient to achieve a concentration of about 0.1 μM, 1 μM, 2 μM, 3 μM, 4 μM, 5 μM, 6 μM, 7 μM, 8 μM, 9 μM, 10 μM, 12 μM, 14 μM, 16 μM, 18 μM or 20 μM in the perilymph fluid in the inner ear and AZD1080 is administered, in amount sufficient to achieve a concentration of about is about 1 μM, 2 μM, 3 μM, 4 μM, 5 μM, 6 μM, 7 μM, 8 μM, 9 μM, or 10 μM, in the perilymph fluid in the inner ear. Alternatively, the Tranylcypromine to a subject, for example to the middle ear at a concentration of about 0.1 mM, 0.2 mM, 0.3 mM, 0.4 mM, 0.5 mM, 0.6 mM, 0.7 mM, 0.8 mM, 0.9 mM, 1 mM, 2 mM, 3 mM, 4 mM, 5 mM, 6 mM, 7 mM, 8 mM, 9 mM, 10 mM, 12 mM, 14 mM, 16 mM, 18 mM, or 20 mM and AZD1080, and is administered to a subject, for example to the middle ear at a concentration of about 1 mM, 2 mM, 3 mM, 4 mM, 5 mM, 6 mM, 7 mM, 8 mM, 9 mM, or 10 mM.

In some embodiments the LSD-1 inhibitor is Tranylcypromine and the Wnt agonist is LY209031. In some embodiments, Tranylcypromine is administered, in amount sufficient to achieve a concentration of about 0.1 μM, 1 μM, 2 μM, 3 μM, 4 μM, 5 μM, 6 μM, 7 μM, 8 μM, 9 μM, 10 μM, 12 μM, 14 μM, 16 μM, 18 μM or 20 μM in the perilymph fluid in the inner ear and LY2090314 is administered, in amount sufficient to achieve a concentration of about 1 nM, 5 nM, 10 nM, 15 nM, 20 nM or 40 nM, in the perilymph fluid in the inner ear. Alternatively, the Tranylcypromine to a subject, for example to the middle ear at a concentration of about 0.1 mM, 0.2 mM, 0.3 mM, 0.4 mM, 0.5 mM, 0.6 mM, 0.7 mM, 0.8 mM, 0.9 mM, 1 mM, 2 mM, 3 mM, 4 mM, 5 mM, 6 mM, 7 mM, 8 mM, 9 mM, 10 mM, 12 mM, 14 mM, 16 mM, 18 mM, or 20 mM and LY2090314, and is administered to a subject, for example to the middle ear at a concentration of about 1 μM, 5 μM, 10 μM, 15 μM, 20 μM, or 40 nM.

In some embodiments the LSD-1 inhibitor is Tranylcypromine and the Wnt agonist is a substituted 3-Imidazo[1,2-a]pyridin-3-yl-4-(1,2,3,4-tetrahydro-[1,4]diazepino-[6,7,1-hi]indol-7-yl)pyrrole-2,5-dione. In some embodiments, Tranylcypromine is administered, in amount sufficient to achieve a concentration of about 0.1 μM, 1 μM, 2 μM, 3 μM, 4 μM, 5 μM, 6 μM, 7 μM, 8 μM, 9 μM, 10 μM, 12 μM, 14 μM, 16 μM, 18 μM, or 20 μM in the perilymph fluid in the inner ear and the substituted 3-Imidazo[1,2-a]pyridin-3-yl-4-(1,2,3,4-tetrahydro-[1,4]diazepino-[6,7,1-hi]indol-7-yl)pyrrole-2,5-dione is administered, in amount sufficient to achieve a concentration of about 1 nM, 5 nM, 10 nM, 15 nM, 20 nM, 50 nM, 100 nM, 250 nM, or 500 nM, in the perilymph fluid in the inner ear. Alternatively, the Tranylcypromine to a subject, for example to the middle ear at a concentration of about 0.1 mM, 0.2 mM, 0.3 mM, 0.4 mM, 0.5 mM, 0.6 mM, 0.7 mM, 0.8 mM, 0.9 mM, 1 mM, 2 mM, 3 mM, 4 mM, 5 mM, 6 mM, 7 mM, 8 mM, 9 mM, 10 mM, 12 mM, 14 mM, 16 mM, 18 mM or 20 mM and the substituted 3-Imidazo[1,2-a]pyridin-3-yl-4-(1,2,3,4-tetrahydro-[1,4]diazepino-[6,7,1-hi]indol-7-yl)pyrrole-2,5-dione and is administered to a subject, for example to the middle ear at a concentration of about 1 μM, 5 μM, 10 μM, 15 μM, 20 μM, 50 μM, 100 μM, 250 μM, 500 μM.

In some embodiments the LSD-1 inhibitor is Tranylcypromine and the Wnt agonist is GSK3 inhibitor XXII. In some embodiments, Tranylcypromine is administered, in amount sufficient to achieve a concentration of about 0.1 μM, 1 μM, 2 μM, 3 μM, 4 μM, 5 μM, 6 μM, 7 μM, 8 μM, 9 μM, 10 μM, 12 μM, 14 μM, 16 μM, 18 μM, or 20 μM in the perilymph fluid in the inner ear and GSK3-inhibitor XXII is administered, in amount sufficient to achieve a concentration of about 0.1 μM, 0.2 μM, 0.3 μM, 0.4 μM, 0.5 μM, 0.6 μM, 0.7 μM, 0.8 μM, 0.9 μM, or 1.0 μM, in the perilymph fluid in the inner ear. Alternatively, the Tranylcypromine to a subject, for example to the middle ear at a concentration of about 0.1 mM, 0.2 mM, 0.3 mM, 0.4 mM, 0.5 mM, 0.6 mM, 0.7 mM, 0.8 mM, 0.9 mM, 1 mM, 2 mM, 3 mM, 4 mM, 5 mM, 6 mM, 7 mM, 8 mM, 9 mM, 10 mM, 12 mM, 14 mM, 16 mM, 18 mM, or 20 mM and the GSK3-inhibitor XXII is administered, in amount sufficient to achieve a concentration of about 0.1 mM, 0.2 mM, 0.3 mM, 0.4 mM, 0.5 mM, 0.6 mM, 0.7 mM, 0.8 mM, 0.9 mM, or 1.0 mM, in the perilymph fluid in the inner ear.

In some embodiments the LSD-1 inhibitor is Tranylcypromine and the Wnt agonist is CHIR99021. In some embodiments, Tranylcypromine is administered, in amount sufficient to achieve a concentration of about 0.1 μM, 1 μM, 2 μM, 3 μM, 4 μM, 5 μM, 6 μM, 7 μM, 8 μM, 9 μM, 10 μM, 12 μM, 14 μM, 16 μM, 18 μM or 20 μM in the perilymph fluid in the inner ear and CHIR99021 is administered, in amount sufficient to achieve a concentration of about 1 μM, 2 μM, 3 μM, 4 μM, 5 μM, 6 μM, 7 μM, 8 μM, 9 μM, or 10 μM, in the perilymph fluid in the inner ear Alternatively, the Tranylcypromine to a subject, for example to the middle ear at a concentration of about 0.1 mM, 0.2 mM, 0.3 mM, 0.4 mM, 0.5 mM, 0.6 mM, 0.7 mM, 0.8 mM, 0.9 mM, 1 mM, 2 mM, 3 mM, 4 mM, 5 mM, 6 mM, 7 mM, 8 mM, 9 mM, 10 mM, 12 mM, 14 mM, 16 mM, 18 mM, or 20 mM. and CHIR99021 is administered to a subject, for example to the middle ear at a concentration of about 1 mM, 2 mM, 3 mM, 4 mM, 5 mM, 6 mM, 7 mM, 8 mM, 9 mM, or 10 mM.

In some embodiments the LSD-1 inhibitor is Phenelzine sulfate and the Wnt agonist is AZD1080. In some embodiments, Phenelzine sulfate is administered, in amount sufficient to achieve a concentration of about 0.1 μM, 0.2 μM, 0.3 μM, 0.4 μM, 0.5 μM, 0.6 μM, 0.7 μM, 0.8 μM, 0.9 μM, 1 μM, 2 μM, 3 μM, 4 μM, 5 μM, 6 μM, 7 μM, 8 μM, 9 μM, or 10 μM in the perilymph fluid in the inner ear and AZD1080 is administered, in amount sufficient to achieve a concentration of about is about 1 μM, 2 μM, 3 μM, 4 μM, 5 μM, 6 μM, 7 μM, 8 μM, 9 μM, or 10 μM, in the perilymph fluid in the inner ear. Alternatively, Phenelzine sulfate is administered to a subject, for example to the middle ear at a concentration of about 0.1 mM, 0.2 mM, 0.3 mM, 0.4 mM, 0.5 mM, 0.6 mM, 0.7 mM, 0.8 mM, 0.9 mM, 1 mM, 2 mM, 3 mM, 4 mM, 5 mM, 6 mM, 7 mM, 8 mM, 9 mM, or 10 mM and AZD1080, and is administered to a subject, for example to the middle ear at a concentration of about 1 mM, 2 mM, 3 mM, 4 mM, 5 mM, 6 mM, 7 mM, 8 mM, 9 mM, or 10 mM.

In some embodiments the LSD-1 inhibitor is Phenelzine sulfate and the Wnt agonist is LY209031. In some embodiments, Phenelzine sulfate is administered, in amount sufficient to achieve a concentration of about 0.1 μM, 0.2 μM, 0.3 μM, 0.4 μM, 0.5 μM, 0.6 μM, 0.7 μM, 0.8 μM, 0.9 μM, 1 μM, 2 μM, 3 μM, 4 μM, 5 μM, 6 μM, 7 μM, 8 μM, 9 μM, or 10 μM in the perilymph fluid in the inner ear and LY2090314 is administered, in amount sufficient to achieve a concentration of about 1 nM, 5 nM, 10 nM, 15 nM, 20 nM or 40 nM in the perilymph fluid in the inner ear. Alternatively, Phenelzine sulfate is administered to a subject, for example to the middle ear at a concentration of about 0.1 mM, 0.2 mM, 0.3 mM, 0.4 mM, 0.5 mM, 0.6 mM, 0.7 mM, 0.8 mM, 0.9 mM, 1 mM, 2 mM, 3 mM, 4 mM, 5 mM, 6 mM, 7 mM, 8 mM, 9 mM, or 10 mM and LY2090314, and is administered to a subject, for example to the middle ear at a concentration of about 1 μM, 5 μM, 10 μM, 15 μM, 20 μM, or 40 μM.

In some embodiments the LSD-1 inhibitor is Phenelzine sulfate and the Wnt agonist is a substituted 3-Imidazo[1,2-a]pyridin-3-yl-4-(1,2,3,4-tetrahydro-[1,4]diazepino-[6,7,1-hi]indol-7-yl)pyrrole-2,5-dione. In some embodiments, Phenelzine sulfate is administered, in amount sufficient to achieve a concentration of about 0.1 μM, 0.2 μM, 0.3 μM, 0.4 μM, 0.5 μM, 0.6 μM, 0.7 μM, 0.8 μM, 0.9 μM, 1 μM, 2 μM, 3 μM, 4 μM, 5 μM, 6 μM, 7 μM, 8 μM, 9 μM, or 10 μM in the perilymph fluid in the inner ear and the substituted 3-Imidazo[1,2-a]pyridin-3-yl-4-(1,2,3,4-tetrahydro-[1,4]diazepino-[6,7,1-hi]indol-7-yl)pyrrole-2,5-dione is administered, in amount sufficient to achieve a concentration of about 1 nM, 5 nM, 10 nM, 15 nM, 20 nM, 50 nM, 100 nM, 250 nM, or 500 nM, in the perilymph fluid in the inner ear. Alternatively, Phenelzine sulfate is administered to a subject, for example to the middle ear at a concentration of about 0.1 mM, 0.2 mM, 0.3 mM, 0.4 mM, 0.5 mM, 0.6 mM, 0.7 mM, 0.8 mM, 0.9 mM, 1 mM, 2 mM, 3 mM, 4 mM, 5 mM, 6 mM, 7 mM, 8 mM, 9 mM, or 10 mM and the substituted 3-Imidazo[1,2-a]pyridin-3-yl-4-(1,2,3,4-tetrahydro-[1,4]diazepino-[6,7,1-hi]indol-7-yl)pyrrole-2,5-dione and is administered to a subject, for example to the middle ear at a concentration of about 1 μM, 5 μM, 10 μM, 15 μM, 20 μM, 50 μM, 100 μM, 250 μM or 500 μM.

In some embodiments the LSD-1 inhibitor is Phenelzine sulfate and the Wnt agonist is GSK3 inhibitor XXII. In some embodiments, Phenelzine sulfate is administered, in amount sufficient to achieve a concentration of about 0.1 μM, 0.2 μM, 0.3 μM, 0.4 uM, 0.5 μM, 0.6 μM, 0.7 μM, 0.8 μM, 0.9 μM, 1 μM, 2 μM, 3 μM, 4 μM, 5 μM, 6 μM, 7 μM, 8 μM, 9 μM, or 10 μM in the perilymph fluid in the inner ear and GSK3-inhibitor XXII is administered, in amount sufficient to achieve a concentration of about 0.1 μM, 0.2 μM, 0.3 μM, 0.4 μM, 0.5 μM, 0.6 μM, 0.7 μM, 0.8 μM, 0.9 μM, or 1.0 μM, in the perilymph fluid in the inner ear Alternatively, Phenelzine sulfate is administered to a subject, for example to the middle ear at a concentration of about 0.1 mM, 0.2 mM, 0.3 mM, 0.4 mM, 0.5 mM, 0.6 mM, 0.7 mM, 0.8 mM, 0.9 mM, 1 mM, 2 mM, 3 mM, 4 mM, 5 mM, 6 mM, 7 mM, 8 mM, 9 mM, or 10 mM and, the GSK3-inhibitor XXII is administered, in amount sufficient to achieve a concentration of about 0.1 mM, 0.2 mM, 0.3 mM, 0.4 mM, 0.5 mM, 0.6 mM, 0.7 mM, 0.8 mM, 0.9 mM, or 1.0 mM, in the perilymph fluid in the inner ear. In some embodiments the LSD-1 inhibitor is Phenelzine sulfate and the Wnt agonist is CHIR99021. In some embodiments, Phenelzine sulfate is administered, in amount sufficient to achieve a concentration of about 0.1 μM, 0.2 μM, 0.3 μM, 0.4 uM, 0.5 μM, 0.6 μM, 0.7 μM, 0.8 μM, 0.9 μM, 1 μM, 2 μM, 3 μM, 4 μM, 5 μM, 6 μM, 7 μM, 8 μM, 9 μM, or 10 μM in the perilymph fluid in the inner ear and CHIR99021 is administered, in amount sufficient to achieve a concentration of about 1 μM, 2 μM, 3 μM, 4 μM, 5 μM, 6 μM, 7 μM, 8 μM, 9 μM, or 10 μM, in the perilymph fluid in the inner ear. Alternatively, Phenelzine sulfate is administered to a subject, for example to the middle ear at a concentration of about 0.1 mM, 0.2 mM, 0.3 mM, 0.4 mM, 0.5 mM, 0.6 mM, 0.7 mM, 0.8 mM, 0.9 mM, 1 mM, 2 mM, 3 mM, 4 mM, 5 mM, 6 mM, 7 mM, 8 mM, 9 mM, or 10 mM and CHIR99021 is administered to a subject, for example to the middle ear at a concentration of about 1 mM, 2 mM, 3 mM, 4 mM, 5 mM, 6 mM, 7 mM, 8 mM, 9 mM, or 10 mM.

In some embodiments the LSD-1 inhibitor is GSK-2879552, the Wnt agonist is AZD1080 and the second epigenetic agent is VPA. In some embodiments, GSK-2879552 is administered, in amount sufficient to achieve a concentration of about 0.1 nM, 0.2 nM, 0.3 nM, 0.4 nM, 0.5 nM, 0.6 nM, 0.7 nM, 0.8 nM, 0.9 nM, 1.0 nM, 2.0 nM, 3.0 nM, 4.0 nM, 5.0 nM, 6.0 nM, 7.0 nM, 8.0 nM, 9.0 nM, 10 nM, 20 nM, 30 nM, 40 nM, 50 nM, 60 nM, 70 nM, 80 nM, 90 nM, 100 nM, 200 nM, 300 nM, 400 nM, 500 nM, 600 nM, 700 nM, 800 nM, 900 nM, 1 μM, 2 μM, 3 μM, 4 μM, 5 μM, 6 μM, 7 μM, 8 μM, 9 μM, 10 μM, 12 μM, 14 μM, 16 μM, 18 μM, 20 μM, 25 μM or about 30 μM in the perilymph fluid in the inner ear; AZD1080 is administered, in amount sufficient to achieve a concentration of about is about 1 μM, 2 μM, 3 μM, 4 μM, 5 μM, 6 μM, 7 μM, 8 μM, 9 μM, or 10 μM, in the perilymph fluid in the inner ear and VPA is administered in amount sufficient to achieve a concentration of about is about 100 μM to 4 mM in the perilymph fluid in the inner ear. Alternatively, the GSK-2879552 is administered to a subject, for example to the middle ear at a concentration of about 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, 10 mM, 12 mM, 14 mM, 16 mM, 18 mM, 20 mM, 25 mM, or about 30 mM; AZD1080 is administered to a subject, for example to the middle ear at a concentration of about 1 mM, 2 mM, 3 mM, 4 mM, 5 mM, 6 mM, 7 mM, 8 mM, 9 mM, or 10 mM and VPA to a subject, for example to the middle ear at a concentration about 100 mM to 4,000 mM.

In some embodiments the LSD-1 inhibitor is GSK-2879552, the Wnt agonist is LY2090314 and the second epigenetic agent is VPA. In some embodiments, GSK-2879552 is administered, in amount sufficient to achieve a concentration of about 0.1 nM, 0.2 nM, 0.3 nM, 0.4 nM, 0.5 nM, 0.6 nM, 0.7 nM, 0.8 nM, 0.9 nM, 1.0 nM, 2.0 nM, 3.0 nM, 4.0 nM, 5.0 nM, 6.0 nM, 7.0 nM, 8.0 nM, 9.0 nM, 10 nM, 20 nM, 30 nM, 40 nM, 50 nM, 60 nM, 70 nM, 80 nM, 90 nM, 100 nM, 200 nM, 300 nM, 400 nM, 500 nM, 600 nM, 700 nM, 800 nM, 900 nM, 1 μM, 2 μM, 3 μM, 4 μM, 5 μM, 6 μM, 7 μM, 8 μM, 9 μM, 10 μM, 12 μM, 14 μM, 16 μM, 18 μM, 20 μM, 25 μM, or about 30 μM in the perilymph fluid in the inner ear; LY2090314 is administered, in amount sufficient to achieve a concentration of about 1 nM, 5 nM, 10 nM, 15 nM, 20 nM or 40 nM in the perilymph fluid in the inner ear and VPA is administered in amount sufficient to achieve a concentration of about is about 100 μM to 4 mM in the perilymph fluid in the inner ear. Alternatively, the GSK-2879552 is administered to a subject, for example to the middle ear at a concentration of about 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, 10 mM, 12 mM, 14 mM, 16 mM, 18 mM, 20 mM, 25 mM, or about 30 mM and LY2090314, and is administered to a subject, for example to the middle ear at a concentration of about 1 μM, 5 μM, 10 μM, 15 μM, 20 μM, or 40 μM and VPA is administers to a subject, for example to the middle ear at a concentration about 100 mM to 4,000 mM.

In some embodiments the LSD-1 inhibitor is GSK-2879552 and the Wnt agonist is a substituted 3-Imidazo[1,2-a]pyridin-3-yl-4-(1,2,3,4-tetrahydro-[1,4]diazepino-[6,7,1-hi]indol-7-yl)pyrrole-2,5-dione and the second epigenetic agent is VPA. In some embodiments, GSK-2879552 is administered, in amount sufficient to achieve a concentration of about 0.1 nM, 0.2 nM, 0.3 nM, 0.4 nM, 0.5 nM, 0.6 nM, 0.7 nM, 0.8 nM, 0.9 nM, 1.0 nM, 2.0 nM, 3.0 nM, 4.0 nM, 5.0 nM, 6.0 nM, 7.0 nM, 8.0 nM, 9.0 nM, 10 nM, 20 nM, 30 nM, 40 nM, 50 nM, 60 nM, 70 nM, 80 nM, 90 nM, 100 nM, 200 nM, 300 nM, 400 nM, 500 nM, 600 nM, 700 nM, 800 nM, 900 nM, 1 μM, 2 μM, 3 μM, 4 μM, 5 μM, 6 μM, 7 μM, 8 μM, 9 μM, 10 μM, 12 μM, 14 μM, 16 μM, 18 μM, 20 μM, 25 μM, or about 30 μM in the perilymph fluid in the inner ear; the substituted 3-Imidazo[1,2-a]pyridin-3-yl-4-(1,2,3,4-tetrahydro-[1,4]diazepino-[6,7,1-hi]indol-7-yl)pyrrole-2,5-dione is administered, in amount sufficient to achieve a concentration of about 1 nM, 5 nM, 10 nM, 15 nM, 20 nM, 50 nM, 100 nM, 250 nM, or 500 nM, in the perilymph fluid in the inner ear and VPA is administered in amount sufficient to achieve a concentration of about is about 100 μM to 4 mM in the perilymph fluid in the inner ear. Alternatively, the GSK-2879552 is administered to a subject, for example to the middle ear at a concentration of about 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, 10 mM, 12 mM, 14 mM, 16 mM, 18 mM, 20 mM, 25 mM, or about 30 mM and the substituted 3-Imidazo[1,2-a]pyridin-3-yl-4-(1,2,3,4-tetrahydro-[1,4]diazepino-[6,7,1-hi]indol-7-yl)pyrrole-2,5-dione and is administered to a subject, for example to the middle ear at a concentration of about 1 μM, 5 μM, 10 μM, 15 μM, 20 μM, 50 μM, 100 μM, 250 μM, or 500 μM and VPA is administers to a subject, for example to the middle ear at a concentration about 100 mM to 4,000 mM.

In some embodiments the LSD-1 inhibitor is GSK-2879552 and the Wnt agonist is GSK3 inhibitor XXII and the second epigenetic agent is VPA. In some embodiments, GSK-2879552 is administered, in amount sufficient to achieve a concentration of about 0.1 nM, 0.2 nM, 0.3 nM, 0.4 nM, 0.5 nM, 0.6 nM, 0.7 nM, 0.8 nM, 0.9 nM, 1.0 nM, 2.0 nM, 3.0 nM, 4.0 nM, 5.0 nM, 6.0 nM, 7.0 nM, 8.0 nM, 9.0 nM, 10 nM, 20 nM, 30 nM, 40 nM, 50 nM, 60 nM, 70 nM, 80 nM, 90 nM, 100 nM, 200 nM, 300 nM, 400 nM, 500 nM, 600 nM, 700 nM, 800 nM, 900 nM, 1 μM, 2 μM, 3 μM, 4 μM, 5 μM, 6 μM, 7 μM, 8 μM, 9 μM, 10 μM, 12 μM, 14 μM, 16 μM, 18 μM, 20 μM, 25 μM, or about 30 μM in the perilymph fluid in the inner ear; the GSK3-inhibitor XXII is administered, in amount sufficient to achieve a concentration of about 0.1 μM, 0.2 μM, 0.3 μM, 0.4 μM, 0.5 μM, 0.6 μM, 0.7 μM, 0.8 μM, 0.9 μM, or 1.0 μM, in the perilymph fluid in the inner ear and VPA is administered in amount sufficient to achieve a concentration of about is about 10 100 μM to 4 mM in the perilymph fluid in the inner ear. Alternatively, the GSK-2879552 is administered to a subject, for example to the middle ear at a concentration of about 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, 10 mM, 12 mM, 14 mM, 16 mM, 18 mM, 20 mM, 25 mM, or about 30 mM and the GSK3-inhibitor XXII is administered, in amount sufficient to achieve a concentration of about 0.1 mM, 0.2 mM, 0.3 mM, 0.4 mM, 0.5 mM, 0.6 mM, 0.7 mM, 0.8 mM, 0.9 mM, or 1.0 mM, in the perilymph fluid in the inner ear and VPA is administers to a subject, for example to the middle ear at a concentration about 100 mM to 4,000 mM.

In some embodiments the LSD-1 inhibitor is GSK-2879552 and the Wnt agonist is CHIR99021 and the second epigenetic agent is VPA. In some embodiments, GSK-2879552 is administered, in amount sufficient to achieve a concentration of about 0.1 nM, 0.2 nM, 0.3 nM, 0.4 nM, 0.5 nM, 0.6 nM, 0.7 nM, 0.8 nM, 0.9 nM, 1.0 nM, 2.0 nM, 3.0 nM, 4.0 nM, 5.0 nM, 6.0 nM, 7.0 nM, 8.0 nM, 9.0 nM, 10 nM, 20 nM, 30 nM, 40 nM, 50 nM, 60 nM, 70 nM, 80 nM, 90 nM, 100 nM, 200 nM, 300 nM, 400 nM, 500 nM, 600 nM, 700 nM, 800 nM, 900 nM, 1 μM, 2 μM, 3 μM, 4 μM, 5 μM, 6 μM, 7 μM, 8 μM, 9 μM, 10 μM, 12 μM, 14 μM, 16 μM, 18 μM, 20 μM, 25 μM, or about 30 μM in the perilymph fluid in the inner ear; CHIR99021 is administered, in amount sufficient to achieve a concentration of about 1 μM, 2 μM, 3 μM, 4 μM, 5 μM, 6 μM, 7 μM, 8 μM, 9 μM, or 10 μM, in the perilymph fluid in the inner ear and VPA is administered in amount sufficient to achieve a concentration of about is about 100 μM to 4 mM in the perilymph fluid in the inner ear. Alternatively, the GSK-2879552 is administered to a subject, for example to the middle ear at a concentration of about 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, 10 mM, 12 mM, 14 mM, 16 mM, 18 mM, 20 mM, 25 mM, or about 30 mM and CHIR99021 is administered to a subject, for example to the middle ear at a concentration of about 1 mM, 2 mM, 3 mM, 4 mM, 5 mM, 6 mM, 7 mM, 8 mM, 9 mM, or 10 mM and VPA is administers to a subject, for example to the middle ear at a concentration about 100 mM to 4,000 mM.

In some embodiments the LSD-1 inhibitor is GSK-LSD1 and the Wnt agonist is AZD1080 and the second epigenetic agent is VPA. In some embodiments, GSK-LSD1 is administered, in amount sufficient to achieve a concentration of about 0.1 nM, 0.2 nM, 0.3 nM, 0.4 nM, 0.5 nM, 0.6 nM, 0.7 nM, 0.8 nM, 0.9 nM, 1.0 nM, 2.0 nM, 3.0 nM, 4.0 nM, 5.0 nM, 6.0 nM, 7.0 nM, 8.0 nM, 9.0 nM, 10 nM, 20 nM, 30 nM, 40 nM, 50 nM, 60 nM, 70 nM, 80 nM, 90 nM, 100 nM, 200 nM, 300 nM, 400 nM, 500 nM, 1 μM, 5 μM, 10 μM, or 50 μM in the perilymph fluid in the inner ear; AZD1080 is administered, in amount sufficient to achieve a concentration of about is about 1 μM, 2 μM, 3 μM, 4 μM, 5 μM, 6 μM, 7 μM, 8 μM, 9 μM, or 10 μM, in the perilymph fluid in the inner ear and VPA is administered in amount sufficient to achieve a concentration of about is about 100 μM to 4 mM in the perilymph fluid in the inner ear. Alternatively, the GSK-LSD1 is administered to a subject, for example to the middle ear at a concentration of about 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 1 mM, 5 mM, 10 mM, or 50 mM and AZD1080, and is administered to a subject, for example to the middle ear at a concentration of about 1 mM, 2 mM, 3 mM, 4 mM, 5 mM, 6 mM, 7 mM, 8 mM, 9 mM, or 10 mM and VPA is administers to a subject, for example to the middle ear at a concentration about 100 mM to 4,000 mM.

In some embodiments the LSD-1 inhibitor is GSK-LSD1 and the Wnt agonist is LY2090314 and the second epigenetic agent is VPA. In some embodiments, GSK-LSD1 is administered, in amount sufficient to achieve a concentration of about 0.1 nM, 0.2 nM, 0.3 nM, 0.4 nM, 0.5 nM, 0.6 nM, 0.7 nM, 0.8 nM, 0.9 nM, 1.0 nM, 2.0 nM, 3.0 nM, 4.0 nM, 5.0 nM, 6.0 nM, 7.0 nM, 8.0 nM, 9.0 nM, 10 nM, 20 nM, 30 nM, 40 nM, 50 nM, 60 nM, 70 nM, 80 nM, 90 nM, 100 nM, 200 nM, 300 nM, 400 nM, 500 nM, 1 μM, 5 μM, 10 μM, or 50 μM in the perilymph fluid in the inner ear; LY2090314 is administered, in amount sufficient to achieve a concentration of about 1 nM, 5 nM, 10 nM, 15 nM, 20 nM or 40 nM in the perilymph fluid in the inner ear and VPA is administered in amount sufficient to achieve a concentration of about is about 100 μM to 4 mM in the perilymph fluid in the inner ear. Alternatively, the GSK-LSD1 is administered to a subject, for example to the middle ear at a concentration of about 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 1 mM, 5 mM, 10 mM, or 50 mM and LY2090314, and is administered to a subject, for example to the middle ear at a concentration of about 1 μM, 5 μM, 10 μM, 15 μM, 20 μM or 40 μM and VPA is administers to a subject, for example to the middle ear at a concentration about 100 mM to 4,000 mM.

In some embodiments the LSD-1 inhibitor is GSK-LSD1 and the Wnt agonist is a substituted 3-Imidazo[1,2-a]pyridin-3-yl-4-(1,2,3,4-tetrahydro-[1,4]diazepino-[6,7,1-hi]indol-7-yl)pyrrole-2,5-dione and the second epigenetic agent is VPA. In some embodiments, GSK-LSD1 is administered, in amount sufficient to achieve a concentration of about 0.1 nM, 0.2 nM, 0.3 nM, 0.4 nM, 0.5 nM, 0.6 nM, 0.7 nM, 0.8 nM, 0.9 nM, 1.0 nM, 2.0 nM, 3.0 nM, 4.0 nM, 5.0 nM, 6.0 nM, 7.0 nM, 8.0 nM, 9.0 nM, 10 nM, 20 nM, 30 nM, 40 nM, 50 nM, 60 nM, 70 nM, 80 nM, 90 nM, 100 nM, 200 nM, 300 nM, 400 nM, 500 nM, 1 μM, 5 μM, 10 μM, or 50 μM in the perilymph fluid in the inner ear; the substituted 3-Imidazo[1,2-a]pyridin-3-yl-4-(1,2,3,4-tetrahydro-[1,4]diazepino-[6,7,1-hi]indol-7-yl)pyrrole-2,5-dione is administered, in amount sufficient to achieve a concentration of about 1 nM, 5 nM, 10 nM, 15 nM, 20 nM, 50 nM, 100 nM, 250 nM, or 500 nM in the perilymph fluid in the inner ear and VPA is administered in amount sufficient to achieve a concentration of about is about 100 μM to 4 mM in the perilymph fluid in the inner ear. Alternatively, the GSK-LSD1 is administered to a subject, for example to the middle ear at a concentration of about 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 1 mM, 5 mM, 10 mM, or 50 mM and the substituted 3-Imidazo[1,2-a]pyridin-3-yl-4-(1,2,3,4-tetrahydro-[1,4]diazepino-[6,7,1-hi]indol-7-yl)pyrrole-2,5-dione and is administered to a subject, for example to the middle ear at a concentration of about 1 μM, 5 μM, 10 μM, 15 μM, 20 μM, 50 μM, 100 μM, 250 μM, or 500 μM and VPA is administers to a subject, for example to the middle ear at a concentration about 100 mM to 4,000 mM.

In some embodiments the LSD-1 inhibitor is GSK-LSD1 and the Wnt agonist is GSK3 inhibitor XXII and the second epigenetic agent is VPA. In some embodiments, GSK-LSD1 is administered, in amount sufficient to achieve a concentration of about 0.1 nM, 0.2 nM, 0.3 nM, 0.4 nM, 0.5 nM, 0.6 nM, 0.7 nM, 0.8 nM, 0.9 nM, 1.0 nM, 2.0 nM, 3.0 nM, 4.0 nM, 5.0 nM, 6.0 nM, 7.0 nM, 8.0 nM, 9.0 nM, 10 nM, 20 nM, 30 nM, 40 nM, 50 nM, 60 nM, 70 nM, 80 nM, 90 nM, 100 nM, 200 nM, 300 nM, 400 nM, 500 nM, 1 μM, 5 μM, 10 μM, or 50 μM in the perilymph fluid in the inner ear; GSK3-inhibitor XXII is administered, in amount sufficient to achieve a concentration of about 0.1 μM, 0.2 μM, 0.3 μM, 0.4 μM, 0.5 μM, 0.6 μM, 0.7 μM, 0.8 μM, 0.9 μM, or 1.0 μM, in the perilymph fluid in the inner ear and VPA is administered in amount sufficient to achieve a concentration of about is about 100 μM to 4 mM in the perilymph fluid in the inner ear. Alternatively, the GSK-LSD1 is administered to a subject, for example to the middle ear at a concentration of about 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM 1 mM, 5 mM, 10 mM, or 50 mM and the GSK3-inhibitor XXII is administered to a subject, for example to the middle ear at a concentration about 0.1 mM, 0.2 mM, 0.3 mM, 0.4 mM, 0.5 mM, 0.6 mM, 0.7 mM, 0.8 mM, 0.9 mM, or 1.0 mM and VPA is administered to a subject, for example to the middle ear at a concentration about 100 mM to 4,000 mM.

In some embodiments the LSD-1 inhibitor is GSK-LSD1 and the Wnt agonist is CHIR99021 and the second epigenetic agent is VPA. In some embodiments, GSK-LSD1 is administered, in amount sufficient to achieve a concentration of about 0.1 nM, 0.2 nM, 0.3 nM, 0.4 nM, 0.5 nM, 0.6 nM, 0.7 nM, 0.8 nM, 0.9 nM, 1.0 nM, 2.0 nM, 3.0 nM, 4.0 nM, 5.0 nM, 6.0 nM, 7.0 nM, 8.0 nM, 9.0 nM, 10 nM, 2.0 nM, 30 nM, 40 nM, 50 nM, 60 nM, 70 nM, 80 nM, 90 nM, 100 nM, 200 nM, 300 nM, 400 nM, 500 nM, 1 μM, 5 μM, 10 μM, or 50 μM in the perilymph fluid in the inner ear; CHIR99021 is administered, in amount sufficient to achieve a concentration of about 1 μM, 2 μM, 3 μM, 4 μM, 5 μM, 6 μM, 7 μM, 8 μM, 9 μM, or 10 μM, in the perilymph fluid in the inner ear and VPA is administered in amount sufficient to achieve a concentration of about is about 100 μM to 4 mM in the perilymph fluid in the inner ear. Alternatively, the GSK-LSD1 is administered to a subject, for example to the middle ear at a concentration of about 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 1 mM, 5 mM, 10 mM, or 50 mM and CHIR99021 is administered to a subject, for example to the middle ear at a concentration of about 1 mM, 2 mM, 3 mM, 4 mM, 5 mM, 6 mM, 7 mM, 8 mM, 9 mM, or 10 mM and VPA is administered to a subject, for example to the middle ear at a concentration about 100 mM to 4,000 mM.

In some embodiments the LSD-1 inhibitor is Tranylcypromine and the Wnt agonist is AZD1080 and the second epigenetic agent is VPA. In some embodiments, Tranylcypromine is administered, in amount sufficient to achieve a concentration of about 0.1 μM, 1 μM, 2 μM, 3 μM, 4 μM, 5 μM, 6 μM, 7 μM, 8 μM, 9 μM, 10 μM, 12 μM, 14 μM, 16 μM, 18 μM or 20 μM in the perilymph fluid in the inner ear and AZD1080 is administered, in amount sufficient to achieve a concentration of about is about 1 μM, 2 μM, 3 μM, 4 μM, 5 μM, 6 μM, 7 μM, 8 μM, 9 μM, or 10 μM in the perilymph fluid in the inner ear and VPA is administered in amount sufficient to achieve a concentration of about is about 100 μM to 4 mM in the perilymph fluid in the inner ear. Alternatively, the Tranylcypromine to a subject, for example to the middle ear at a concentration of about 0.1 mM, 0.2 mM, 0.3 mM, 0.4 mM, 0.5 mM, 0.6 mM, 0.7 mM, 0.8 mM, 0.9 mM, 1 mM, 2 mM, 3 mM, 4 mM, 5 mM, 6 mM, 7 mM, 8 mM, 9 mM, 10 mM, or 10 mM and AZD1080, and is administered to a subject, for example to the middle ear at a concentration of about 1 mM, 2 mM, 3 mM, 4 mM, 5 mM, 6 mM, 7 mM, 8 mM, 9 mM, or 10 mM and VPA is administered to a subject, for example to the middle ear at a concentration about 100 mM to 4,000 mM.

In some embodiments the LSD-1 inhibitor is Tranylcypromine and the Wnt agonist is LY209031 and the second epigenetic agent is VPA. In some embodiments, Tranylcypromine is administered, in amount sufficient to achieve a concentration of about 0.11 μM, 1 μM, 2 μM, 3 μM, 4 μM, 5 μM, 6 μM, 7 μM, 8 μM, 9 μM, 10 μM, 12 μM, 14 μM, 16 μM, 18 μM or 20 μM in the perilymph fluid in the inner ear and LY2090314 is administered, in amount sufficient to achieve a concentration of about 1 nM, 5 nM, 10 nM, 15 nM, 20 nM or 40 nM in the perilymph fluid in the inner ear and VPA is administered in amount sufficient to achieve a concentration of about is about 100 μM to 4 mM in the perilymph fluid in the inner ear. Alternatively, the Tranylcypromine to a subject, for example to the middle ear at a concentration of about 0.1 mM, 0.2 mM, 0.3 mM, 0.4 mM, 0.5 mM, 0.6 mM, 0.7 mM, 0.8 mM, 0.9 mM, 1 mM, 2 mM, 3 mM, 4 mM, 5 mM, 6 mM, 7 mM, 8 mM, 9 mM, 10 mM, 12 mM, 14 mM, 16 mM, 18 mM, or 20 mM and LY2090314, and is administered to a subject, for example to the middle ear at a concentration of about 1 μM, 5 μM, 10 μM, 15 μM, or 20 μM and VPA is administered to a subject, for example to the middle ear at a concentration about 100 mM to 4,000 mM.

In some embodiments the LSD-1 inhibitor is Tranylcypromine and the Wnt agonist is a substituted 3-Imidazo[1,2-a]pyridin-3-yl-4-(1,2,3,4-tetrahydro-[1,4]diazepino-[6,7,1-hi]indol-7-yl)pyrrole-2,5-dione and the second epigenetic agent is IPA. In some embodiments, Tranylcypromine is administered, in amount sufficient to achieve a concentration of about 0.1 μM, 1 μM, 2 μM, 3 μM, 4 μM, 5 μM, 6 μM, 7 μM, 8 μM, 9 μM, 10 μM, 12 μM, 14 μM, 16 μM, 18 μM or 20 μM in the perilymph fluid in the inner ear and the substituted 3-Imidazo[1,2-a]pyridin-3-yl-4-(1,2,3,4-tetrahydro-[1,4]diazepino-[6,7,1-hi]indol-7-yl)pyrrole-2,5-dione is administered, in amount sufficient to achieve a concentration of about 1 nM, 5 nM, 10 nM, 15 nM, 20 nM, 50 nM, 100 nM, 250 nM, or 500 nM, in the perilymph fluid in the inner ear and VPA is administered in amount sufficient to achieve a concentration of about is about 100 μM to 4 mM in the perilymph fluid in the inner ear. Alternatively, the Tranylcypromine to a subject, for example to the middle ear at a concentration of about 0.1 mM, 0.2 mM, 0.3 mM, 0.4 mM, 0.5 mM, 0.6 mM, 0.7 mM, 0.8 mM, 0.9 mM, 1 mM, 2 mM, 3 mM, 4 mM, 5 mM, 6 mM, 7 mM, 8 mM, 9 mM, 10 mM, 12 mM, 14 mM, 16 mM, 18 mM, or 20 mM and the substituted 3-Imidazo[1,2-a]pyridin-3-yl-4-(1,2,3,4-tetrahydro-[1,4]diazepino-[6,7,1-hi]indol-7-yl)pyrrole-2,5-dione and is administered to a subject, for example to the middle ear at a concentration of about 1 μM, 5 μM, 10 μM, 15 μM, 20 μM, 50 μM, 100 μM, 250 μM, or 500 μM and VPA is administered to a subject, for example to the middle ear at a concentration about 100 mM to 4,000 mM.

In some embodiments the LSD-1 inhibitor is Tranylcypromine and the Wnt agonist is GSK3 inhibitor XXII and the second epigenetic agent is VPA. In some embodiments, Tranylcypromine is administered, in amount sufficient to achieve a concentration of about 0.1 μM, 1 μM, 2 μM, 3 μM, 4 μM, 5 μM, 6 μM, 7 μM, 8 μM, 9 μM, 10 μM, 12 μM, 14 μM, 16 μM, 18 μM or 20 μM in the perilymph fluid in the inner ear and GSK3-inhibitor XXII is administered, in amount sufficient to achieve a concentration of about 0.1 μM, 0.2 μM, 0.3 μM, 0.4 μM, 0.5 μM, 0.6 μM, 0.7 μM, 0.8 μM, 0.9 μM, or 1.0 μM, in the perilymph fluid in the inner ear and VPA is administered in amount sufficient to achieve a concentration of about is about 100 μM to 4 mM in the perilymph fluid in the inner ear. Alternatively, the Tranylcypromine to a subject, for example to the middle ear at a concentration of about 0.1 mM, 0.2 mM, 0.3 mM, 0.4 mM, 0.5 mM, 0.6 mM, 0.7 mM, 0.8 mM, 0.9 mM, 1 mM, 2 mM, 3 mM, 4 mM, 5 mM, 6 mM, 7 mM, 8 mM, 9 mM, 10 mM, 12 mM, 14 mM, 16 mM, 18 mM, or 20 mM and the GSK3-inhibitor XXII is administered, in amount sufficient to achieve a concentration of about 0.1 mM, 0.2 mM, 0.3 mM, 0.4 mM, 0.5 mM, 0.6 mM, 0.7 mM, 0.8 mM, 0.9 mM, or 1.0 mM in the perilymph fluid in the inner ear and VPA is administered to a subject, for example to the middle ear at a concentration about 100 mM to 4,000 mM.

In some embodiments the LSD-1 inhibitor is Tranylcypromine and the Wnt agonist is CHIR99021 and the second epigenetic agent is VPA. In some embodiments, Tranylcypromine is administered, in amount sufficient to achieve a concentration of about 0.1 μM, 1 μM, 2 μM, 3 μM, 4 μM, 5 μM, 6 μM, 7 μM, 8 μM, 9 μM, 10 μM, 12 μM, 14 μM, 16 μM, 18 μM or 20 μM in the perilymph fluid in the inner ear and CHIR99021 is administered, in amount sufficient to achieve a concentration of about 1 μM, 2 μM, 3 μM, 4 μM, 5 μM, 6 μM, 7 μM, 8 μM, 9 μM, or 10 μM, in the perilymph fluid in the inner ear and VPA is administered in amount sufficient to achieve a concentration of about is about 100 μM to 4 mM in the perilymph fluid in the inner ear. Alternatively, the Tranylcypromine to a subject, for example to the middle ear at a concentration of about 0.1 mM, 0.2 mM, 0.3 mM, 0.4 mM, 0.5 mM, 0.6 mM, 0.7 mM, 0.8 mM, 0.9 mM, 1 mM, 2 mM, 3 mM, 4 mM, 5 mM, 6 mM, 7 mM, 8 mM, 9 mM, 10 mM, 12 mM, 14 mM, 16 mM, 18 mM, or 20 mM and CHIR99021 is administered to a subject, for example to the middle ear at a concentration of about 1 mM, 2 mM, 3 mM, 4 mM, 5 mM, 6 mM, 7 mM, 8 mM, 9 mM, or 10 mM and VPA is administered to a subject, for example to the middle ear at a concentration about 100 mM to 4,000 mM.

In some embodiments the LSD-1 inhibitor is Phenelzine sulfate and the Wnt agonist is AZD1080 and the second epigenetic agent is VPA. In some embodiments, Phenelzine sulfate is administered, in amount sufficient to achieve a concentration of about 0.1 μM, 0.2 μM, 0.3 μM, 0.4 μM, 0.5 μM, 0.6 μM, 0.7 μM, 0.8 μM, 0.9 μM, 1 μM, 2 μM, 3 μM, 4 μM, 5 μM, 6 μM, 7 μM, 8 μM, 9 μM, or 10 μM in the perilymph fluid in the inner ear and AZD1080 is administered, in amount sufficient to achieve a concentration of about is about 1 μM, 2 μM, 3 μM, 4 μM, 5 μM, 6 μM, 7 μM, 8 μM, 9 μM, or 10 μM in the perilymph fluid in the inner ear and VPA is administered in amount sufficient to achieve a concentration of about is about 100 μM to 4 mM in the perilymph fluid in the inner ear. Alternatively, Phenelzine sulfate is administered to a subject, for example to the middle ear at a concentration of about 0.1 mM, 0.2 mM, 0.3 mM, 0.4 mM, 0.5 mM, 0.6 mM, 0.7 mM, 0.8 mM, 0.9 mM, 1 mM, 2 mM, 3 mM, 4 mM, 5 mM, 6 mM, 7 mM, 8 mM, 9 mM, or 10 mM and AZD1080, and is administered to a subject, for example to the middle ear at a concentration of about 1 mM, 2 mM, 3 mM, 4 mM, 5 mM, 6 mM, 7 mM, 8 mM, 9 mM, or 10 mM and VPA is administered to a subject, for example to the middle ear at a concentration about 100 mM to 4,000 mM.

In some embodiments the LSD-1 inhibitor is Phenelzine sulfate and the Wnt agonist is LY209031 and the second epigenetic agent is WA. In some embodiments, Phenelzine sulfate is administered, in amount sufficient to achieve a concentration of about 0.1 μM, 0.2 μM, 0.3 μM, 0.4 μM, 0.5 μM, 0.6 μM, 0.7 μM, 0.8 μM, 0.9 μM, 1 μM, 2 μM, 3 μM, 4 μM, 5 μM, 6 μM, 7 μM, 8 μM, 9 μM, or 10 μM in the perilymph fluid in the inner ear and LY2090314 is administered, in amount sufficient to achieve a concentration of about 1 nM, 5 nM, 10 nM, 15 nM, 20 nM or 40 nM, in the perilymph fluid in the inner ear and VPA is administered in amount sufficient to achieve a concentration of about is about 100 μM to 4 mM in the perilymph fluid in the inner ear. Alternatively, Phenelzine sulfate is administered to a subject, for example to the middle ear at a concentration of about 0.1 mM, 0.2 mM, 0.3 mM, 0.4 mM, 0.5 mM, 0.6 mM, 0.7 mM, 0.8 mM, 0.9 mM, 1 mM, 2 mM, 3 mM, 4 mM, 5 mM, 6 mM, 7 mM, 8 mM, 9 mM, or 10 mM and LY2090314, and is administered to a subject, for example to the middle ear at a concentration of about 1 μM, 5 μM, 10 μM, 15 μM, 20 μM, or 40 μM and VPA is administered to a subject, for example to the middle ear at a concentration about 100 mM to 4,000 mM.

In some embodiments the LSD-1 inhibitor is Phenelzine sulfate and the Wnt agonist is a substituted 3-Imidazo[1,2-a]pyridin-3-yl-4-(1,2,3,4-tetrahydro-[1,4]diazepino-[6,7,1-hi]indol-7-yl)pyrrole-2,5-dione and the second epigenetic agent is VPA. In some embodiments, Phenelzine sulfate is administered, in amount sufficient to achieve a concentration of about 0.1 μM, 0.2 μM, 0.3 μM, 0.4 μM, 0.5 μM, 0.6 μM, 0.7 μM, 0.8 μM, 0.9 μM, 1 μM, 2 μM, 3 μM, 4 μM, 5 μM, 6 μM, 7 μM, 8 μM, 9 μM, or 10 μM in the perilymph fluid in the inner ear and the substituted 3-Imidazo[1,2-a]pyridin-3-yl-4-(1,2,3,4-tetrahydro-[1,4]diazepino-[6,7,1-hi]indol-7-yl)pyrrole-2,5-dione is administered, in amount sufficient to achieve a concentration of about 1 nM, 5 nM, 10 nM, 15 nM, 20 nM, 50 nM, 100 nM, 250 nM, or 500 nM, in the perilymph fluid in the inner ear and VPA is administered in amount sufficient to achieve a concentration of about is about 100 μM to 4 mM in the perilymph fluid in the inner ear. Alternatively, Phenelzine sulfate is administered to a subject, for example to the middle ear at a concentration of about 0.1 mM, 0.2 mM, 0.3 mM, 0.4 mM, 0.5 mM, 0.6 mM, 0.7 mM, 0.8 mM, 0.9 mM, 1 mM, 2 mM, 3 mM, 4 mM, 5 mM, 6 mM, 7 mM, 8 mM, 9 mM, or 10 mM and the substituted 3-Imidazo[1,2-a]pyridin-3-yl-4-(1,2,3,4-tetrahydro-[1,4]diazepino-[6,7,1-hi]indol-7-yl)pyrrole-2,5-dione and is administered to a subject, for example to the middle ear at a concentration of about 1 μM, 5 μM, 10 μM, 15 μM, 20 μM, 50 μM, 100 μM, 250 μM or 500 μM and VPA is administered to a subject, for example to the middle ear at a concentration about 100 mM to 4,000 mM

In some embodiments the LSD-1 inhibitor is Phenelzine sulfate and the Wnt agonist is GSK3 inhibitor XXII and the second epigenetic agent is VPA. In some embodiments, Phenelzine sulfate is administered, in amount sufficient to achieve a concentration of about 0.1 μM, 0.2 μM, 0.3 μM, 0.4 μM, 0.5 μM, 0.6 μM, 0.7 μM, 0.8 μM, 0.9 μM, 1 μM, 2 μM, 3 μM, 4 μM, 5 μM, 6 μM, 7 μM, 8 μM, 9 μM, or 10 μM in the perilymph fluid in the inner ear and GSK3-inhibitor XXII is administered, in amount sufficient to achieve a concentration of about 0.1 μM, 0.2 μM, 0.3 μM, 0.4 μM, 0.5 μM, 0.6 μM, 0.7 μM, 0.8 μM, 0.9 μM, or 1.0 μM, in the perilymph fluid in the inner ear and VPA is administered in amount sufficient to achieve a concentration of about is about 100 μM to 4 mM in the perilymph fluid in the inner ear. Alternatively, Phenelzine sulfate is administered to a subject, for example to the middle ear at a concentration of about 0.1 mM, 0.2 mM, 0.3 mM, 0.4 mM, 0.5 mM, 0.6 mM, 0.7 mM, 0.8 mM, 0.9 mM, 1 mM, 2 mM, 3 mM, 4 mM, 5 mM, 6 mM, 7 mM, 8 mM, 9 mM, or 10 mM and the GSK3-inhibitor XXII is administered to a subject, for example to the middle ear at a concentration of about 0.1 mM, 0.2 mM, 0.3 mM, 0.4 mM, 0.5 mM, 0.6 mM, 0.7 mM, 0.8 mM, 0.9 mM, or 1.0 mM and VPA is administered to a subject, for example to the middle ear at a concentration about 100 mM to 4,000 mM.

In some embodiments the LSD-1 inhibitor is Phenelzine sulfate and the Wnt agonist is CHIR99021 and the second epigenetic agent is VPA. In some embodiments, Phenelzine sulfate is administered, in amount sufficient to achieve a concentration of about 0.1 μM, 0.2 μM, 0.3 μM, 0.4 μM, 0.5 μM, 0.6 μM, 0.7 μM, 0.8 μM, 0.9 μM, 1 μM, 2 μM, 3 μM, 4 μM, 5 μM, 6 μM, 7 μM, 8 μM, 9 μM, or 10 μM in the perilymph fluid in the inner ear and CHIR99021 is administered, in amount sufficient to achieve a concentration of about 1 μM, 2 μM, 3 μM, 4 μM, 5 μM, 6 μM, 7 μM, 8 μM, 9 μM, or 10 μM, in the perilymph fluid in the inner ear and VPA is administered in amount sufficient to achieve a concentration of about is about 100 μM to 4 mM in the perilymph fluid in the inner ear. Alternatively, Phenelzine sulfate is administered to a subject, for example to the middle ear at a concentration of about 0.1 mM, 0.2 mM, 0.3 mM, 0.4 mM, 0.5 mM, 0.6 mM, 0.7 mM, 0.8 mM, 0.9 mM, 1 mM, 2 mM, 3 mM, 4 mM, 5 mM, 6 mM, 7 mM, 8 mM, 9 mM, or 10 mM and CHIR99021 is administered to a subject, for example to the middle ear at a concentration of about 1 mM, 2 mM, 3 mM, 4 mM, 5 mM, 6 mM, 7 mM, 8 mM, 9 mM, or 10 mM and VPA is administered to a subject, for example to the middle ear at a concentration about 100 mM to 4,000 mM.

In some embodiments the LSD1 inhibitor is ORY-1001 and the Wnt agonist is AZD1080 and the second epigenetic agent is VPA. In some embodiments, ORY-1001 is administered, in amount sufficient to achieve a concentration of about 10 □M, 20 nM, 30 nM, 40 nM, 50 nM, 60 nM, 70 nM, 80 nM, 90 nM, 0.1 μM, 0.2 μM, 0.3 μM, 0.4 μM, 0.5 μM, 0.6 μM, 0.7 μM, 0.8 μM, 0.9 μM, 1 μM, 2 μM, 3 μM, 4 μM, 5 μM, 6 μM, 7 μM, 8 μM, 9 μM, or 10 μM in the perilymph fluid in the inner ear and AZD1080 is administered, in amount sufficient to achieve a concentration of about is about 1 μM, 2 μM, 3 μM, 4 μM, 5 μM, 6 μM, 7 μM, 8 μM, 9 μM, or 10 μM in the perilymph fluid in the inner ear and VPA is administered in amount sufficient to achieve a concentration of about is about 100 μM to 4 mM in the perilymph fluid in the inner ear. Alternatively, ORY-1001 is administered to a subject, for example to the middle ear at a concentration of about 10 μM, 20 μM, 30 μM, 40 μM, 50 μM, 60 μM, 70 μM, 80 μM, 90 μM, 0.1 mM, 0.2 mM, 0.3 mM, 0.4 mM, 0.5 mM, 0.6 mM, 0.7 mM, 0.8 mM, 0.9 mM, 1 mM, 2 mM, 3 mM, 4 mM, 5 mM, 6 mM, 7 mM, 8 mM, 9 mM, or 10 mM and AZD1080, and is administered to a subject, for example to the middle ear at a concentration of about 1 mM, 2 mM, 3 mM, 4 mM, 5 mM, 6 mM, 7 mM, 8 mM, 9 mM, or 10 mM and VPA is administered to a subject, for example to the middle ear at a concentration about 100 mM to 4,000 mM.

In some embodiments the LSD1 inhibitor is ORY-1001 and the Wnt agonist is LY209031 and the second epigenetic agent is VPA. In some embodiments, ORY-1001 is administered, in amount sufficient to achieve a concentration of about 10 nM, 20 nM, 30 nM, 40 nM, 50 nM, 60 nM, 70 nM, 80 nM, 90 nM, 0.1 μM, 0.2 μM, 0.3 μM, 0.4 μM, 0.5 μM, 0.6 μM, 0.7 μM, 0.8 μM, 0.9 μM, 1 μM, 2 μM, 3 μM, 4 μM, 5 μM, 6 μM, 7 μM, 8 μM, 9 μM, or 10 μM in the perilymph fluid in the inner ear and LY2090314 is administered, in amount sufficient to achieve a concentration of about 1 nM, 5 nM, 10 nM, 15 nM, 20 nM or 40 nM, in the perilymph fluid in the inner ear and VPA is administered in amount sufficient to achieve a concentration of about is about 100 μM to 4 mM in the perilymph fluid in the inner ear. Alternatively, ORY-1001 is administered to a subject, for example to the middle ear at a concentration of about 10 μM, 20 μM, 30 μM, 40 μM, 50 μM, 60 μM, 70 μM, 80 μM, 90 μM, 0.1 mM, 0.2 mM, 0.3 mM, 0.4 mM, 0.5 mM, 0.6 mM, 0.7 mM, 0.8 mM, 0.9 mM, 1 mM, 2 mM, 3 mM, 4 mM, 5 mM, 6 mM, 7 mM, 8 mM, 9 mM, or 10 mM and LY2090314, and is administered to a subject, for example to the middle ear at a concentration of about 1 μM, 5 μM, 10 μM, 15 μM, 20 μM, or 40 μM and VPA is administered to a subject, for example to the middle ear at a concentration about 100 mM to 4,000 mM,

In some embodiments the LSD1 inhibitor is ORY-1001 and the Wnt agonist is a substituted 3-Imidazo[1,2-a]pyridin-3-yl-4-(1,2,3,4-tetrahydro-[1,4]diazepino-[6,7,1-hi]indol-7-yl)pyrrole-2,5-dione and the second epigenetic agent is IPA. In some embodiments, ORY-1001 is administered, in amount sufficient to achieve a concentration of about 10 nM, 20 nM, 30 nM, 40 nM, 50 nM, 60 nM, 70 nM, 80 nM, 90 nM, 0.1 μM, 0.2 μM, 0.3 μM, 0.4 μM, 0.5 μM, 0.6 μM, 0.7 μM, 0.8 μM, 0.9 μM, 1 μM, 2 μM, 3 μM, 4 μM, 5 μM, 6 μM, 7 μM, 8 μM, 9 μM, or 10 μM in the perilymph fluid in the inner ear and the substituted 3-Imidazo[1,2-a]pyridin-3-yl-4-(1,2,3,4-tetrahydro-[1,4]diazepino-[6,7,1-hi]indol-7-yl)pyrrole-2,5-dione is administered, in amount sufficient to achieve a concentration of about 1 nM, 5 nM, 10 nM, 15 nM, 20 nM, 50 nM, 100 nM, 250 nM, or 500 nM, in the perilymph fluid in the inner ear and VPA is administered in amount sufficient to achieve a concentration of about is about 100 μM to 4 mM in the perilymph fluid in the inner ear. Alternatively, ORY-1001 is administered to a subject, for example to the middle ear at a concentration of about 10 μM, 20 μM, 30 μM, 40 μM, 50 μM, 60 μM, 70 μM, 80 μM, 90 μM, 0.1 mM, 0.2 mM, 0.3 mM, 0.4 mM, 0.5 mM, 0.6 mM, 0.7 mM, 0.8 mM, 0.9 mM, 1 mM, 2 mM, 3 mM, 4 mM, 5 mM, 6 mM, 7 mM, 8 mM, 9 mM, or 10 mM and the substituted 3-Imidazo[1,2-a]pyridin-3-yl-4-(1,2,3,4-tetrahydro-[1,4]diazepino-[6,7,1-hi]indol-7-yl)pyrrole-2,5-dione and is administered to a subject, for example to the middle ear at a concentration of about 1 μM, 5 μM, 10 μM, 15 μM, 20 μM, 50 μM, 100 μM, 250 μM or 500 μM and VPA is administered to a subject, for example to the middle ear at a concentration about 100 mM to 4,000 mM.

In some embodiments the LSD1 inhibitor is ORY-1001 and the Wnt agonist is GSK3 inhibitor XXII and the second epigenetic agent is VPA. In some embodiments, ORY-1001 is administered, in amount sufficient to achieve a concentration of about 10 nM, 20 nM, 30 nM, 40 nM, 50 nM, 60 nM, 70 nM, 80 nM, 90 nM, 0.1 μM, 0.2 μM, 0.3 μM, 0.4 μM, 0.5 μM, 0.6 μM, 0.7 μM, 0.8 μM, 0.9 μM, 1 μM, 2 μM, 3 μM, 4 μM, 5 μM, 6 μM, 7 μM, 8 μM, 9 μM, or 10 μM in the perilymph fluid in the inner ear and GSK3-inhibitor XXII is administered, in amount sufficient to achieve a concentration of about 0.1 μM, 0.2 μM, 0.3 μM, 0.4 μM, 0.5 μM, 0.6 μM, 0.7 μM, 0.8 μM, 0.9 μM, or 1.0 μM, in the perilymph fluid in the inner ear and TA is administered in amount sufficient to achieve a concentration of about is about 100 μM to 4 mM in the perilymph fluid in the inner ear. Alternatively, ORY-1001 is administered to a subject, for example to the middle ear at a concentration of about 10 μM, 20 μM, 30 μM, 40 μM, 50 μM, 60 μM, 70 μM, 80 μM, 90 μM, 0.1 mM, 0.2 mM, 0.3 mM, 0.4 mM, 0.5 mM, 0.6 mM, 0.7 mM, 0.8 mM, 0.9 mM, 1 mM, 2 mM, 3 mM, 4 mM, 5 mM, 6 mM, 7 mM, 8 mM, 9 mM, or 10 mM and the GSK3-inhibitor XXII is administered to a subject, for example to the middle ear at a concentration of about 0.1 mM, 0.2 mM, 0.3 mM, 0.4 mM, 0.5 mM, 0.6 mM, 0.7 mM, 0.8 mM, 0.9 mM, or 1.0 mM and VPA is administered to a subject, for example to the middle ear at a concentration about 100 mM to 4,000 mM.

In some embodiments the LSD1 inhibitor is ORY-1001 and the Wnt agonist is CHIR99021 and the second epigenetic agent is VPA. In some embodiments, ORY-1001 is administered, in amount sufficient to achieve a concentration of about 10 nM, 20 nM, 30 nM, 40 nM, 50 nM, 60 nM, 70 nM, 80 nM, 90 nM, 0.1 μM, 0.2 μM, 0.3 μM, 0.4 μM, 0.5 μM, 0.6 μM, 0.7 μM, 0.8 μM, 0.9 μM, 1 μM, 2 μM, 3 μM, 4 μM, 5 μM, 6 μM, 7 μM, 8 μM, 9 μM, or 10 μM in the perilymph fluid in the inner ear and CHIR99021 is administered, in amount sufficient to achieve a concentration of about 1 μM, 2 μM, 3 μM, 4 μM, 5 μM, 6 μM, 7 μM, 8 μM, 9 μM, or 10 μM, in the perilymph fluid in the inner ear and VPA is administered in amount sufficient to achieve a concentration of about is about 100 μM to 4 mM in the perilymph fluid in the inner ear. Alternatively, ORY-1001 is administered to a subject, for example to the middle ear at a concentration of about 10 μM, 20 μM, 30 μM, 40 μM, 50 μM, 60 μM, 70 μM, 80 μM, 90 μM, 0.1 mM, 0.2 mM, 0.3 mM, 0.4 mM, 0.5 mM, 0.6 mM, 0.7 mM, 0.8 mM, 0.9 mM, 1 mM, 2 mM, 3 mM, 4 mM, 5 mM, 6 mM, 7 mM, 8 mM, 9 mM, or 10 mM and CHIR99021 is administered to a subject, for example to the middle ear at a concentration of about 1 mM, 2 mM, 3 mM, 4 mM, 5 mM, 6 mM, 7 mM, 8 mM, 9 mM, or 10 mM and VPA is administered to a subject, for example to the middle ear at a concentration about 100 mM to 4,000 mM.

In some embodiments the ISD1 inhibitor is RN-1 and the Wnt agonist is AZD1080 and the second epigenetic agent is VPA. In some embodiments, RN-1 is administered, in amount sufficient to achieve a concentration of about 10 nM, 20 nM, 30 nM, 40 nM, 50 nM, 60 nM, 70 nM, 80 nM, 90 nM, 0.1 μM, 0.2 μM, 0.3 μM, 0.4 μM, 0.5 μM, 0.6 μM, 0.7 μM, 0.8 μM, 0.9 μM, 1 μM, 2 μM, 3 μM, 4 μM, 5 μM, 6 μM, 7 μM, 8 μM, 9 μM, or 10 μM in the perilymph fluid in the inner ear and AZD1080 is administered, in amount sufficient to achieve a concentration of about is about 1 μM, 2 μM, 3 μM, 4 μM, 5 μM, 6 μM, 7 μM, 8 μM, 9 μM, or 10 μM in the perilymph fluid in the inner ear and VPA is administered in amount sufficient to achieve a concentration of about is about 100 μM to 4 mM in the perilymph fluid in the inner ear. Alternatively, RN-1 is administered to a subject, for example to the middle ear at a concentration of about 10 μM, 20 μM, 30 μM, 40 μM, 50 μM, 60 μM, 70 μM, 80 μM, 90 μM, 0.1 mM, 0.2 mM, 0.3 mM, 0.4 mM, 0.5 mM, 0.6 mM, 0.7 mM, 0.8 mM, 0.9 mM, 1 mM, 2 mM, 3 mM, 4 mM, 5 mM, 6 mM, 7 mM, 8 mM, 9 mM, or 10 mM and AZD1080, and is administered to a subject, for example to the middle ear at a concentration of about 1 mM, 2 mM, 3 mM, mM, 5 mM, 6 mM, 7 mM, 8 mM, 9 mM, or 10 mM and VPA is administered to a subject, for example to the middle ear at a concentration about 100 mM to 4,000 mM.

In some embodiments the LSD1 inhibitor is RN-1 and the Wnt agonist is LY209031 and the second epigenetic agent is VPA. In some embodiments, RN-1 is administered, in amount sufficient to achieve a concentration of about 10 nM, 20 nM, 30 nM, 40 nM, 50 nM, 60 nM, 70 nM, 80 nM, 90 nM, 0.1 μM, 0.2 μM, 0.3 μM, 0.4 μM, 0.5 μM, 0.6 μM, 0.7 μM, 0.8 μM, 0.9 μM, 1 μM, 2 μM, 3 μM, 4 μM, 5 μM, 6 μM, 7 μM, 8 μM, 9 μM, or 10 μM in the perilymph fluid in the inner ear and LY2090314 is administered, in amount sufficient to achieve a concentration of about 1 nM, 5 nM, 10 nM, 15 nM, 20 nM or 40 nM, in the perilymph fluid in the inner ear and VPA is administered in amount sufficient to achieve a concentration of about is about 100 μM to 4 mM in the perilymph fluid in the inner ear. Alternatively, RN-1 is administered to a subject, for example to the middle ear at a concentration of about 10 μM, 20 μM, 30 μM, 40 μM, 50 μM, 60 μM, 70 μM, 80 μM, 90 μM, 0.1 mM, 0.2 mM, 0.3 mM, 0.4 mM, 0.5 mM, 0.6 mM, 0.7 mM, 0.8 mM, 0.9 mM, 1 mM, 2 mM, 3 mM, 4 mM, 5 mM, 6 mM, 7 mM, 8 mM, 9 mM, or 10 mM and LY2090314, and is administered to a subject, for example to the middle ear at a concentration of about 1 μM, 5 μM, 10 μM, 15 μM, 20 μM, or 40 μM and VPA is administered to a subject, for example to the middle ear at a concentration about 100 mM to 4,000 mM.

In some embodiments the LSD1 inhibitor is RN-1 and the Wnt agonist is a substituted 3-Imidazo[1,2-a]pyridin-3-yl-4-(1,2,3,4-tetrahydro-[1,4]diazepino-[6,7,1-hi]indol-7-yl)pyrrole-2,5-dione and the second epigenetic agent is VPA. In some embodiments, RN-1 is administered, in amount sufficient to achieve a concentration of about 10 nM, 20 nM, 30 nM, 40 nM, 50 nM, 60 nM, 70 nM, 80 nM, 90 nM, 0.1 μM, 0.2 μM, 0.3 μM, 0.4 μM, 0.5 μM, 0.6 μM, 0.7 μM, 0.8 μM, 0.9 μM, 1 μM, 2 μM, 3 μM, 4 μM, 5 μM, 6 μM, 7 μM, 8 μM, 9 μM, or 10 μM in the perilymph fluid in the inner ear and the substituted 3-Imidazo[1,2-a]pyridin-3-yl-4-(1,2,3,4-tetrahydro-[1,4]diazepino-[6,7,1-hi]indol-7-yl)pyrrole-2,5-dione is administered, in amount sufficient to achieve a concentration of about 1 nM, 5 nM, 10 nM, 15 nM, 20 nM, 50 nM, 100 nM, 250 nM, or 500 nM, in the perilymph fluid in the inner ear and VPA is administered in amount sufficient to achieve a concentration of about is about 100 μM to 4 mM in the perilymph fluid in the inner ear. Alternatively, RN-1 is administered to a subject, for example to the middle ear at a concentration of about 10 μM, 20 μM, 30 μM, 40 μM, 50 μM, 60 μM, 70 μM, 80 μM, 90 μM, 0.1 mM, 0.2 mM, 0.3 mM, 0.4 mM, 0.5 mM, 0.6 mM, 0.7 mM, 0.8 mM, 0.9 mM, 1 mM, 2 mM, 3 mM, 4 mM, 5 mM, 6 mM, 7 mM, 8 mM, 9 mM, or 10 mM and the substituted 3-Imidazo[1,2-a]pyridin-3-yl-4-(1,2,3,4-tetrahydro-[1,4]diazepino-[6,7,1-hi]indol-7-yl)pyrrole-2,5-dione and is administered to a subject, for example to the middle ear at a concentration of about 1 μM, 5 μM, 10 μM, 15 μM, 20 μM, 50 μM, 100 μM, 250 μM or 500 μM and VPA is administered to a subject, for example to the middle ear at a concentration about 100 mM to 4,000 mM.

In some embodiments the LSD1 inhibitor is RN-1 and the Wnt agonist is GSK3 inhibitor XXII and the second epigenetic agent is VPA. In some embodiments, RN-1 is administered, in amount sufficient to achieve a concentration of about 10 nM, 20 nM, 30 nM, 40 nM, 50 nM, 60 nM, 70 nM, 80 nM, 90 nM, 0.1 μM, 0.2 μM, 0.3 μM, 0.4 μM, 0.5 μM, 0.6 μM, 0.7 μM, 0.8 μM, 0.9 μM, 1 μM, 2 μM, 3 μM, 4 μM, 5 μM, 6 μM, 7 μM, 8 μM, 9 μM, or 10 μM in the perilymph fluid in the inner ear and GSK3-inhibitor XXII is administered, in amount sufficient to achieve a concentration of about 0.1 μM, 0.2 μM, 0.3 μM, 0.4 μM, 0.5 μM, 0.6 μM, 0.7 μM, 0.8 μM, 0.9 μM, or 1.0 μM, in the perilymph fluid in the inner ear and VPA is administered in amount sufficient to achieve a concentration of about is about 100 μM to 4 mM in the perilymph fluid in the inner ear. Alternatively, RN-1 is administered to a subject, for example to the middle ear at a concentration of about 10 μM, 20 μM, 30 μM, 40 μM, 50 μM, 60 μM, 70 μM, 80 μM, 90 μM, 0.1 mM, 0.2 mM, 0.3 mM, 0.4 mM, 0.5 mM, 0.6 mM, 0.7 mM, 0.8 mM, 0.9 mM, 1 mM, 2 mM, 3 mM, 4 mM, 5 mM, 6 mM, 7 mM, 8 mM, 9 mM, or 10 mM and the GSK3-inhibitor XXII is administered to a subject, for example to the middle ear at a concentration of about 0.1 mM, 0.2 mM, 0.3 mM, 0.4 mM, 0.5 mM, 0.6 mM, 0.7 mM, 0.8 mM, 0.9 mM, or 1.0 mM and VPA is administered to a subject, for example to the middle ear at a concentration about 100 mM to 4,000 mM.

In some embodiments the LSD1 inhibitor is RN-1 and the Wnt agonist is CHIR99021 and the second epigenetic agent is VPA. In some embodiments, RN-1 is administered, in amount sufficient to achieve a concentration of about 10 nM, 20 nM, 30 nM, 40 nM, 50 nM, 60 nM, 70 nM, 80 nM, 90 nM, 0.1 μM, 0.2 μM, 0.3 μM, 0.4 μM, 0.5 μM, 0.6 μM, 0.7 μM, 0.8 μM, 0.9 μM, 1 μM, 2 μM, 3 μM, 4 μM, 5 μM, 6 μM, 7 μM, 8 μM, 9 μM, or 10 μM in the perilymph fluid in the inner ear and CHIR99021 is administered, in amount sufficient to achieve a concentration of about 1 μM, 2 μM, 3 μM, 4 μM, 5 μM, 6 μM, 7 μM, 8 μM, 9 μM, or 10 μM, in the perilymph fluid in the inner ear and VPA is administered in amount sufficient to achieve a concentration of about is about 100 μM to 4 mM in the perilymph fluid in the inner ear. Alternatively, RN-1 is administered to a subject, for example to the middle ear at a concentration of about 10 μM, 20 μM, 30 μM, 40 μM, 50 μM, 60 μM, 70 μM, 80 μM, 90 μM, 0.1 mM, 0.2 mM, 0.3 mM, 0.4 mM, 0.5 mM, 0.6 mM, 0.7 mM, 0.8 mM, 0.9 mM, 1 mM, 2 mM, 3 mM, 4 mM, 5 mM, 6 mM, 7 mM, 8 mM, 9 mM, or 10 mM and CHIR99021 is administered to a subject, for example to the middle ear at a concentration of about 1 mM, 2 mM, 3 mM, 4 mM, 5 mM, 6 mM, 7 mM, 8 mM, 9 mM, or 10 mM and VPA is administered to a subject, for example to the middle ear at a concentration about 100 mM to 4,000 mM.

In some embodiments the additional epigenetic agent is an EZH2 inhibitor.

In some embodiments, the EZH2 inhibitor is PF-06821497 and is administered for example to a cochlear cell in amount sufficient to achieve a concentration of about 0.001 nM to 100 μM, about 0.01 nM to 10 μM, about 0.1 nM to 1 μM, about 1 nM to 100 nM, about 1 nM to 10 nM, about 10 nM to 100 nM, or about 100 nM to 1 μM, in the perilymph fluid in the inner ear.

In some embodiments, the PF-06821497 is administered, in amount sufficient to achieve a concentration of about 0.1 nM, 0.2 nM, 0.3 nM, 0.4 nM, 0.5 nM, 0.6 nM, 0.7 nM, 0.8 nM, 0.9 nM, 1.0 nM, 2.0 nM, 3.0 nM, 4.0 nM, 5.0 nM, 6.0 nM, 7.0 nM, 8.0 nM, 9.0 nM, 10 nM, 20 nM, 30 nM, 40 nM, 50 nM, 60 nM, 70 nM, 80 nM, 90 nM, 100 nM, 200 nM, 300 nM, 400 nM, 500 nM, 600 nM, 700 nM, 800 nM, 900 nM, or about 1 μM in the perilymph fluid in the inner ear.

In some embodiments, the EZH2 inhibitor is PF-06821497 is administered to a subject, for example to the middle ear at a concentration of 0.001 μM to 100 mM, about 0.01 μM to 10 mM, about 0.1 μM to 1 mM, about 1 μM to 100 μM, about 1 μM to 10 μM, 10 μM to 100 μM, or about 100 μM to 1 mM.

In some embodiments, the PF-06821497 is administered to a subject, for example to the middle ear at a concentration of about 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, or about 1 mM.

In some embodiments, the EZH2 inhibitor is PF-06821497 and is administered systemically at a daily dose of about 50 mg to 5,000 mg/day, about 50 mg to 4000 mg/day, about 50 mg to 3000 mg/day, about 50 mg to 2000 mg/day, about 50 mg to 1000 mg/day, about 50 mg to 500 mg/day, about 100 mg to 2500 mg/day, about 100 mg to 2000 mg/day, about 100 mg to 1500 mg/day, about 100 mg to 1000 mg/day, about 100 mg to 500 mg/day, about 150 mg to 2500 mg/day, about 150 mg to 2000 mg/day, about 150 mg to 1500 mg/day, about 150 mg to 1250 mg/day, about 75 mg/day, about 100 mg/day, about 150 mg/day, about 200 mg/day, about 300 mg/day, about 400 mg/day, about 500 mg/day, about 600 mg/day, about 700 mg/day, about 800 mg/day, about 900 mg/day, about 1000 mg/day, about 1200 mg/day, about 1400 mg/day, about 1600 mg/day, about 1800 mg/day, or about 2000 mg/day.

In some embodiments, the EZH2 inhibitor is PF-06821497 and is administered to the subject at a concentration ratio of about 0.001 to 100 fold relative to an FDA approved concentration or about 0.01 to 50 fold relative to an FDA approved concentration or about 0.1 to 10 fold relative to an FDA approved concentration, or about 0.1 to 5 fold relative to an FDA approved, or about 1 to 5 fold relative to an FDA approved concentration.

In some embodiments, EZH2 inhibitor is PF-06821497 and is administered to the subject at about 0.01×, 0.1×, 1×, 2×, 3×, 4×, 5× or 10×, relative to an FDA approved dose. A PF-06821497 dose is for example the concentration listed on Table 7, column titled “Human Dosage”.

In some embodiments, the EZH2 inhibitor is CPI-12135 and is administered for example to a cochlear cell in amount sufficient to achieve a concentration of about 0.01 nM to 1 mM, about 0.1 nM to 100 μM, about 1 nM to 10 μM, about 10 nM to 1 μM, about 1 nM to 10 nM, about 10 nM to 100 nM, or about 100 nM to 1 μM, in the perilymph fluid in the inner ear.

In some embodiments, the CPI-1205 is administered, in amount sufficient to achieve a concentration of about 1.0 nM, 2.0 nM, 3.0 nM, 4.0 nM, 5.0 nM, 6.0 nM, 7.0 nM, 8.0 nM, 9.0 nM, 10 nM, 20 nM, 30 nM, 40 nM, 50 nM, 60 nM, 70 nM, 80 nM, 90 nM, 100 nM, 200 nM, 300 nM, 400 nM, 500 nM, 600 nM, 700 nM, 800 nM, 900 nM, or about 1 μM, in the perilymph fluid in the inner ear.

In some embodiments, the EZH2 inhibitor is CPI-1205 is administered to a subject, for example to the middle ear at a concentration of 0.001 μM to 100 mM, about 0.01 μM to 10 mM, about 0.1 μM to 1 mM, about 1 μM to 100 μM, about 1 μM to 10 μM, about 10 μM to 100 μM, or about 100 μM to 1000 μM.

In some embodiments, the CPI-1205 is administered to a subject, for example to the middle ear at a concentration of about 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, or about 1 mM.

In some embodiments, the EZH2 inhibitor is CPI-1205 and is administered systemically at a daily dose of about 100 to 5,000 mg/day, about 100 mg to 4000 mg/day, about 100 mg to 3000 mg/day, about 100 mg to 2000 mg/day, about 500 to 5,000 mg/day, about 500 mg to 4000 mg/day, about 500 mg to 3000 mg/day, about 750 to 5,000 mg/day, about 750 mg to 4000 mg/day, about 750 mg to 3000 mg/day, about 800 mg to 2400 mg/day, about 400 mg/day, about 600 mg/day, about 800 mg/day, about 1000 mg/day, about 1200 mg/day, about 1400 mg/day, about 1600 mg/day, about 1800 mg/day, about 2000 mg/day, about 2200 mg/day, about 2400 mg/day, about 2600 mg/day, about 2800 mg/day, about 3000 mg/day, about 3250 mg/day, about 3500 mg/day, about 4000 mg/day, about 4500 mg/day, or about 5000 mg/day.

In some embodiments, the EZH2 inhibitor is CPI-1205 and is administered to the subject at a concentration ratio of about 0.001 to 100 fold relative to an FDA approved concentration or about 0.01 to 50 fold relative to an FDA approved concentration or about 0.1 to 10 fold relative to an FDA approved concentration, or about 0.1 to 5 fold relative to an FDA approved, or about 1 to 5 fold relative to an FDA approved concentration.

In some embodiments, EZH2 inhibitor is CPI-1205 and is administered to the subject at about 0.01×. 0.1×, 1×, 2×, 3×, 4×, 5× or 10×, relative to an FDA approved dose. A CPI-1205 dose is for example the concentration listed on Table 7, column titled “Human Dosage”.

In some embodiments, the EZH2 inhibitor is valemetostat and is administered for example to a cochlear cell in amount sufficient to achieve a concentration of about 0.01 nM to 1 mM, about 0.1 nM to 100 μM, about 1 nM to 10 μM, about 10 nM to 1 μM, about 1 nM to 10 nM, about 10 nM to 100 nM, or about 100 nM to 1 μM, in the perilymph fluid in the inner ear.

In some embodiments, the valemetostat is administered, in amount sufficient to achieve a concentration of about 1.0 nM, 2.0 nM, 3.0 nM, 4.0 nM, 5.0 nM, 6.0 nM, 7.0 nM, 8.0 nM, 9.0 nM, 10 nM, 20 nM, 30 nM, 40 nM, 50 nM, 60 nM, 70 nM, 80 nM, 90 nM, 100 nM, 200 nM, 300 nM, 400 nM, 500 nM, 600 nM, 700 nM, 800 nM, 900 nM, or 1 μM, in the perilymph fluid in the inner ear.

In some embodiments, the EZH2 inhibitor is valemetostat is administered to a subject, for example to the middle ear at a concentration of about 0.001 μM to 100 mM, about 0.01 μM, to 10 mM, about 0.1 μM to 1 mM, about 1 μM to 100 μM, about 1 μM to 10 μM, 10 μM to 100 μM, or about 100 μM to 1000 μM.

In some embodiments, the valemetostat is administered to a subject, for example to the middle ear at a concentration of about 0.1 μM, 0.2 μM, 0.3 μM, 0.4 μM, 0.5 μM, 0.64M, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, or 1 mM.

In some embodiments, the EZH2 inhibitor is valemetostat and is administered systemically at a daily dose of about 50 mg to 5,000 mg/day, about 50 mg to 4000 mg/day, about 50 mg to 3000 mg/day, about 50 mg to 2000 mg/day, about 50 mg to 1000 mg/day, about 50 mg to 500 mg/day, about 100 mg to 2000 mg/day, about 100 mg to 1500 mg/day, about 100 mg to 1000 mg/day, about 100 mg to 500 mg/day, about 100 mg/day, about 200 mg/day, about 300 mg/day, about 400 mg/day, about 500 mg/day, about 600 mg/day, about 700 mg/day, about 800 mg/day, about 900 mg/day, about 1000 mg/day, about 1200 mg/day, about 1400 mg/day, about 1600 mg/day, about 1800 mg/day, or about 2000 mg/day.

In some embodiments, the EZH2 inhibitor is valemetostat and is administered to the subject at a concentration ratio of about 0.001 to 100 fold relative to an FDA approved concentration or about 0.01 to 50 fold relative to an FDA approved concentration or about 0.1 to 10 fold relative to an FDA approved concentration, or about 0.1 to 5 fold relative to an FDA approved, or about 1 to 5 fold relative to an FDA approved concentration.

In some embodiments, EZH2 inhibitor is valemetostat and is administered to the subject at about 0.01×. 0.1×, 1×, 2×, 3×, 4×, 5× or 10×, relative to an FDA approved dose. A valemetostat dose is for example the concentration listed on Table 7, column titled “Human Dosage”

In some embodiments, the EZH2 inhibitor is tazemetostat and is administered for example to a cochlear cell in amount sufficient to achieve a concentration of about 0.01 nM to 1 mM, about 0.1 nM to 100 μM, about 1 nM to 10 μM, about 10 nM to 1 μM, about 1 nM to 10 nM, about 10 nM to 100 nM, 100 nM to 1 μM, or about 1 μM to 10 μM, in the perilymph fluid in the inner ear.

In some embodiments, the tazemetostat is administered, in amount sufficient to achieve a concentration of about 10 nM, 20 nM, 30 nM, 40 nM, 50 nM, 60 nM, 70 nM, 80 nM, 90 nM, 100 nM, 200 nM, 300 nM, 400 nM, 500 nM, 600 nM, 700 nM, 800 nM, 900 nM, 1 μM, 2 μM, 3 μM, 4 μM, 5 μM, 6 μM, 7 μM, 8 μM, 9 μM, or about 10 μM, in the perilymph fluid in the inner ear.

In some embodiments, the EZH2 inhibitor is tazemetostat is administered to a subject, for example to the middle ear at a concentration of about 0.001 μM to 100 mM, about 0.01 μM to 10 mM, about 0.1 μM to 1 mM, about 1 μM to 100 μM, about 1 μM to 10 μM, 10 μM to 100 μM, about 100 μM to 1000 μM or about 1 mM to 10 mM.

In some embodiments, the tazemetostat is administered to a subject, for example to the middle ear at a concentration of about 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, or 10 mM.

In some embodiments, the EZH2 inhibitor is tazemetostat and is administered systemically at a daily dose of about 50 mg to 5,000 mg/day, about 50 mg to 4000 mg/day, about 50 mg to 3000 mg/day, about 50 mg to 2000 mg/day, about 50 mg to 1000 mg/day, about 50 mg to 500 mg/day, about 100 mg to 2500 mg/day, about 100 mg to 2000 mg/day, about 100 mg to 1500 mg/day, about 100 mg to 1000 mg/day, about 100 mg to 500 mg/day, about 200 mg to 2500 mg/day, about 200 mg to 2000 mg/day, about 200 mg to 1600 mg/day, about 200 mg to 1000 mg/day, about 100 mg/day, about 200 mg/day, about 300 mg/day, about 400 mg/day, about 500 mg/day, about 600 mg/day, about 700 mg/day, about 800 mg/day, about 900 mg/day, about 1000 mg/day, about 1200 mg/day, about 1400 mg/day, about 1600 mg/day, about 1800 mg/day, or about 2000 mg/day.

In some embodiments, the EZH2 inhibitor is tazemetostat and is administered to the subject at a concentration ratio of about 0.001 to 100 fold relative to an FDA approved concentration or about 0.01 to 50 fold relative to an FDA approved concentration or about 0.1 to 10 fold relative to an FDA approved concentration, or about 0.1 to 5 fold relative to an FDA approved, or about 1 to 5 fold relative to an FDA approved concentration.

In some embodiments, EZH2 inhibitor is tazemetostat and is administered to the subject at about 0.01×. 0.1×, 1×, 2×, 3×, 4×, 5× or 10×, relative to an FDA approved dose. A tazemetostat dose is for example the concentration listed on Table 7, column titled “Human Dosage”.

In some embodiments, the EZH2 inhibitor is E11 and is administered for example to a cochlear cell in amount sufficient to achieve a concentration of about 0.1 nM to 1 mM, about 1 nM to 100 μM, about 10 nM to 10 μM, about 100 nM to 10 μM, about 10 nM to 100 nM, about 100 nM to 1 μM, about 1 μM to 10 μM, or about 10 μM to 100 μM, in the perilymph fluid in the inner ear.

In some embodiments, the E11 is administered, in amount sufficient to achieve a concentration of about 10 nM, 20 nM, 30 nM, 40 nM, 50 nM, 60 nM, 70 nM, 80 nM, 90 nM, 100 nM, 200 nM, 300 nM, 400 nM, 500 nM, 600 nM, 700 nM, 800 nM, 900 nM, 1 μM, 2 μM, 3 μM, 4 μM, 5 μM, 6 μM, 7 μM, 8 μM, 9 μM, 10 μM, 15 μM, 20 μM, 25 μM, 30 μM, 35 μM, 40 μM, 45 μM, or about 50 μM, in the perilymph fluid in the inner ear.

In some embodiments, the EZH2 inhibitor is E11 is administered to a subject, for example to the middle ear at a concentration of about 0.1 μM to 1000 mM, about 1 μM to 100 mM, about 10 μM to 10 mM, about 100 μM to 10 mM, about 1 μM to 10 μM, 10 μM to 100 μM, about 100 μM to 1000 μM, 1 mM to 10 mM, or about 10 mM to 100 mM.

In some embodiments, the E11 is administered to a subject, for example to the middle ear at a concentration of about 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, 10 mM, 15 mM, 20 mM, 25 mM, 30 mM, 35 mM, 40 mM, 45 mM, or about 50 mM.

In some embodiments, the EZH2 inhibitor is E11 and is administered systemically at a daily dose of about 50 mg to 5,000 mg/day, about 50 mg to 4000 mg/day, about 50 mg to 3000 mg/day, about 50 mg to 2000 mg/day, about 50 mg to 1000 mg/day, about 50 mg to 500 mg/day, about 100 mg to 2500 mg/day, about 100 mg to 2000 mg/day, about 100 mg to 1500 mg/day, about 100 mg to 1000 mg/day, about 100 mg to 500 mg/day, about 200 mg to 2500 mg/day, about 200 mg to 2000 mg/day, about 200 mg to 1500 mg/day, about 200 mg to 1000 mg/day, about 100 mg/day, about 200 mg/day, about 300 mg/day, about 400 mg/day, about 500 mg/day, about 600 mg/day, about 700 mg/day, about 800 mg/day, about 900 mg/day, about 1000 mg/day, about 1200 mg/day, about 1400 mg/day, about 1600 mg/day, about 1800 mg/day, or about 2000 mg/day.

In some embodiments, the EZH2 inhibitor is E11 and is administered to the subject at a concentration ratio of about 0.001 to 100 fold relative to an FDA approved concentration or about 0.01 to 50 fold relative to an FDA approved concentration or about 0.1 to 10 fold relative to an FDA approved concentration, or about 0.1 to 5 fold relative to an FDA approved, or about 1 to 5 fold relative to an FDA approved concentration.

In some embodiments, EZH2 inhibitor is E11 and is administered to the subject at about 0.01×. 0.1×, 1×, 2×, 3×, 4×, 5× or 10×, relative to an FDA approved dose. An E11 dose is for example the concentration listed on Table 7, column titled “Human Dosage”.

In some embodiments, the EZH2 inhibitor is CPI-169 and is administered for example to a cochlear cell in amount sufficient to achieve a concentration of about 0.1 nM to 1 mM, about 1 nM to 100 μM, about 10 nM to 10 μM, about 100 nM to 10 μM, about 10 nM to 100 nM, about 100 nM to 1 μM, about 1 μM to 10 μM, or about 10 μM to 100 μM, in the perilymph fluid in the inner ear.

In some embodiments, the CPI-169 is administered, in amount sufficient to achieve a concentration of about 10 nM, 20 nM, 30 nM, 40 nM, 50 nM, 60 nM, 70 nM, 80 nM, 90 nM, 100 nM, 200 nM, 300 nM, 400 nM, 500 nM, 600 nM, 700 nM, 800 nM, 900 nM, 1 μM, 2 μM, 3 μM, 4 μM, 5 μM, 6 μM, 7 μM, 8 μM, 9 μM, 10 μM, 15 μM, 20 μM, 25 μM, 30 μM, 35 μM, 40 μM, 45 μM, or about 50 μM, in the perilymph fluid in the inner ear.

In some embodiments, the EZH2 inhibitor is CPI-169 and is administered to the subject at a concentration ratio of about 0.001 to 100 fold relative to an FDA approved concentration or about 0.01 to 50 fold relative to an FDA approved concentration or about 0.1 to 10 fold relative to an FDA approved concentration, or about 0.1 to 5 fold relative to an FDA approved, or about 1 to 5 fold relative to an FDA approved concentration.

In some embodiments, EZH2 inhibitor is CPI-169 and is administered to the subject at about 0.01×. 0.1×, 1×, 2×, 3×, 4×, 5× or 10×, relative to an FDA approved dose. An CPI-169 dose is for example the concentration listed on Table 7, column titled “Human Dosage”.

In some embodiments, the EZH2 inhibitor is CPI-360 and is administered for example to a cochlear cell in amount sufficient to achieve a concentration of about 0.001 nM to 1000 μM, about 0.01 nM to 100 μM, about 0.1 nM to 10 μM, about 1 nM to 1000 nM, about 1 nM to 10 nM, about 10 nM to 100 nM, about 100 nM to 1 μM, about 1000 nM to 10 μM, or about 10 μM to 100 μM, in the perilymph fluid in the inner ear.

In some embodiments, the CPI-360 is administered, in amount sufficient to achieve a concentration of about 0.1 nM, 0.2 nM, 0.3 nM, 0.4 nM, 0.5 nM, 0.6 nM, 0.7 nM, 0.8 nM, 0.9 nM, 1.0 nM, 2.0 nM, 3.0 nM, 4.0 nM, 5.0 nM, 6.0 nM, 7.0 nM, 8.0 nM, 9.0 nM, 10 nM, 20 nM, 30 nM, 40 nM, 50 nM, 60 nM, 70 nM, 80 nM, 90 nM, 100 nM, 200 nM, 300 nM, 400 nM, 500 nM, 600 nM, 700 nM, 800 nM, 900 nM, 1 μM, 2 μM, 3 μM, 4 μM, 5 μM, 6 μM, 7 μM, 8 μM, 9 μM, 10 μM, or about 20 μM in the perilymph fluid in the inner ear.

In some embodiments, the EZH2 inhibitor is CPI-360 is administered to a subject, for example to the middle ear at a concentration of 0.001 μM to 100 mM, about 0.01 μM to 10 mM, about 0.1 μM to 1 mM, about 1 μM to 100 μM, about 1 μM to 10 μM, 10 μM to 100 μM, about 100 μM to 1 mM, 1 mM to 10 mM, or about 10 mM to 100 mM.

In some embodiments, the CPI-360 is administered to a subject, for example to the middle ear at a concentration of about 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, 10 mM, 20 mM, 30 mM, or about 40 mM.

In some embodiments, the EZH2 inhibitor is CPI-360 and is administered systemically at a daily dose of about 50 mg to 5,000 mg/day, about 50 mg to 4000 mg/day, about 50 mg to 3000 mg/day, about 50 mg to 2000 mg/day, about 50 mg to 1000 mg/day, about 50 mg to 500 mg/day, about 100 mg to 2500 mg/day, about 100 mg to 2000 mg/day, about 100 mg to 1500 mg/day, about 100 mg to 1000 mg/day, about 100 mg to 500 mg/day, about 150 mg to 2500 mg/day, about 150 mg to 2000 mg/day, about 150 mg to 1500 mg/day, about 150 mg to 1250 mg/day, about 75 mg/day, about 100 mg/day, about 150 mg/day, about 200 mg/day, about 300 mg/day, about 400 mg/day, about 500 mg/day, about 600 mg/day, about 700 mg/day, about 800 mg/day, about 900 mg/day, about 1000 mg/day, about 1200 mg/day, about 1400 mg/day, about 1600 mg/day, about 1800 mg/day, or about 2000 mg/day.

In some embodiments, the EZH2 inhibitor is CPI-360 and is administered to the subject at a concentration ratio of about 0.001 to 100 fold relative to an FDA approved concentration or about 0.01 to 50 fold relative to an FDA approved concentration or about 0.1 to 10 fold relative to an FDA approved concentration, or about 0.1 to 5 fold relative to an FDA approved, or about 1 to 5 fold relative to an FDA approved concentration.

In some embodiments, the EZH2 inhibitor is EPZ011989 and is administered for example to a cochlear cell in amount sufficient to achieve a concentration of about 0.001 nM to 100 μM, about 0.01 nM to 10 μM, about 0.1 nM to 1 μM, about 1 nM to 100 nM, about 1 nM to 10 nM, about 10 nM to 100 nM, or about 100 nM to 1 μM, in the perilymph fluid in the inner ear.

In some embodiments, the EPZ011989 is administered, in amount sufficient to achieve a concentration of about 0.1 nM, 0.2 nM, 0.3 nM, 0.4 nM, 0.5 nM, 0.6 nM, 0.7 nM, 0.8 nM, 0.9 nM, 1.0 nM, 2.0 nM, 3.0 nM, 4.0 nM, 5.0 nM, 6.0 nM, 7.0 nM, 8.0 nM, 9.0 nM, 10 nM, 20 nM, 30 nM, 40 nM, 50 nM, 60 nM, 70 nM, 80 nM, 90 nM, 100 nM, 200 nM, 300 nM, 400 nM, 500 nM, 600 nM, 700 nM, 800 nM, 900 nM, or about 1 μM in the perilymph fluid in the inner ear.

In some embodiments, the EZH2 inhibitor is EPZ011989 is administered to a subject, for example to the middle ear at a concentration of 0.001 μM to 100 mM, about 0.01 μM to 10 mM, about 0.1 μM to 1 mM, about 1 μM to 100 μM, about 1 μM to 10 μM, 10 μM to 100 μM, or about 100 μM to 1 mM.

In some embodiments, the EZH2 inhibitor is CPI-360 is administered to a subject, for example to the middle ear at a concentration of 0.001 μM to 100 mM, about 0.01 μM to 10 mM, about 0.1 μM to 1 mM, about 1 μM to 100 μM, about 1 μM to 10 μM, 10 μM to 100 μM, or about 100 μM to 1 mM.

In some embodiments, the EPZ011989 is administered to a subject, for example to the middle ear at a concentration of about 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, or about 1 mM.

In some embodiments, the EZH2 inhibitor is EPZ011989 and is administered systemically at a daily dose of about 50 mg to 5,000 mg/day, about 50 mg to 4000 mg/day, about 50 mg to 3000 mg/day, about 50 mg to 2000 mg/day, about 50 mg to 1000 mg/day, about 50 mg to 500 mg/day, about 100 mg to 2500 mg/day, about 100 mg to 2000 mg/day, about 100 mg to 1500 mg/day, about 100 mg to 1000 mg/day, about 100 mg to 500 mg/day, about 150 mg to 2500 mg/day, about 150 mg to 2000 mg/day, about 150 mg to 1500 mg/day, about 150 mg to 1250 mg/day, about 75 mg/day, about 100 mg/day, about 150 mg/day, about 200 mg/day, about 300 mg/day, about 400 mg/day, about 500 mg/day, about 600 mg/day, about 700 mg/day, about 800 mg/day, about 900 mg/day, about 1000 mg/day, about 1200 mg/day, about 1400 mg/day, about 1600 mg/day, about 1800 mg/day, or about 2000 mg/day.

In some embodiments, the EZH2 inhibitor is EPZ011989 and is administered to the subject at a concentration ratio of about 0.001 to 100 fold relative to an FDA approved concentration or about 0.01 to 50 fold relative to an FDA approved concentration or about 0.1 to 10 fold relative to an FDA approved concentration, or about 0.1 to 5 fold relative to an FDA approved, or about 1 to 5 fold relative to an FDA approved concentration.

In some embodiments, the EZH2 inhibitor is UNC 2399 and is administered for example to a cochlear cell in amount sufficient to achieve a concentration of about 0.001 nM to 100 μM, about 0.01 nM to 10 μM, about 0.1 nM to 1 μM, about 1 nM to 100 nM, about 1 nM to 10 nM, about 10 nM to 100 nM, about 100 nM to 1 μM, about 1 μM to 10 μM or about 10 μM to 100 μM, in the perilymph fluid in the inner ear.

In some embodiments, the UNC 2399 is administered, in amount sufficient to achieve a concentration of about 0.1 nM, 0.2 nM, 0.3 nM, 0.4 nM, 0.5 nM, 0.6 nM, 0.7 nM, 0.8 nM, 0.9 nM, 1.0 nM, 2.0 nM, 3.0 nM, 4.0 nM, 5.0 nM, 6.0 nM, 7.0 nM, 8.0 nM, 9.0 nM, 10 nM, 20 nM, 30 nM, 40 nM, 50 nM, 60 nM, 70 nM, 80 nM, 90 nM, 100 nM, 200 nM, 300 nM, 400 nM, 500 nM, 600 nM, 700 nM, 800 nM, 900 nM, 1 μM, 2 μM, 3 μM, 4 μM, 5 μM, 6 μM, 7 μM, 8 μM, 9 μM, 10 μM, 15 μM, 20 μM, 30 μM or about 40 μM in the perilymph fluid in the inner ear.

In some embodiments, the EZH2 inhibitor is UNC 2399 is administered to a subject, for example to the middle ear at a concentration of 0.001 μM to 100 mM, about 0.01 μM to 10 mM, about 0.1 μM to 1 mM, about 1 μM to 100 μM, about 1 μM to 10 μM, 10 μM to 100 μM, about 100 μM to 1 mM, 1 mM to 10 mM, or about 10 mM to 100 mM.

In some embodiments, the UNC 2399 is administered to a subject, for example to the middle ear at a concentration of about 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, 10 mM, 20 mM, 30 mM, or about 40 mM.

In some embodiments, the EZH42 inhibitor is UNC 2399 and is administered systemically at a daily dose of about 50 mg to 5,000 mg/day, about 50 mg to 4000 mg/day, about 50 mg to 3000 mg/day, about 50 mg to 2000 mg/day, about 50 mg to 1000 mg/day, about 50 mg to 500 mg/day, about 100 mg to 2500 mg/day, about 100 mg to 2000 mg/day, about 100 mg to 1500 mg/day, about 100 mg to 1000 mg/day, about 100 mg to 500 mg/day, about 150 mg to 2500 mg/day, about 150 mg to 2000 mg/day, about 150 mg to 1500 mg/day, about 150 mg to 1250 mg/day, about 75 mg/day, about 100 mg/day, about 150 mg/day, about 200 mg/day, about 300 mg/day, about 400 mg/day, about 500 mg/day, about 600 mg/day, about 700 mg/day, about 800 mg/day, about 900 mg/day, about 1000 mg/day, about 1200 mg/day, about 1400 mg/day, about 1600 mg/day, about 1800 mg/day, or about 2000 mg/day.

In some embodiments, the EZH2 inhibitor is UNC 2399 and is administered to the subject at a concentration ratio of about 0.001 to 100 fold relative to an FDA approved concentration or about 0.01 to 50 fold relative to an FDA approved concentration or about 0.1 to 10 fold relative to an FDA approved concentration, or about 0.1 to 5 fold relative to an FDA approved, or about 1 to 5 fold relative to an FDA approved concentration

In some embodiments, the EZH2 inhibitor is PF-06726304 and is administered for example to a cochlear cell in amount sufficient to achieve a concentration of about 0.01 nM to 1 mM, about 0.1 nM to 100 μM, about 1 nM to 10 μM, about 10 nM to 1 μM, about 1 nM to 10 nM, about 10 nM to 100 nM, 100 nM to 1 μM, or about 1 μM to 10 μM, in the perilymph fluid in the inner ear.

In some embodiments, the PF-06726304 is administered, in amount sufficient to achieve a concentration of about 10 nM, 20 nM, 30 nM, 40 nM, 50 nM, 60 nM, 70 nM, 80 nM, 90 nM, 100 nM, 200 nM, 300 nM, 400 nM, 500 nM, 600 nM, 700 nM, 800 mM, 900 nM, 1 μM, 2 μM, 3 μM, 4 μM, 5 μM, 6 μM, 7 μM, 8 μM, 9 μM, or about 10 μM, in the perilymph fluid in the inner ear.

In some embodiments, the EZH2 inhibitor is PF-06726304 is administered to a subject, for example to the middle ear at a concentration of about 0.001 μM to 100 mM, about 0.01 μM to 10 mM, about 0.1 μM to 1 mM, about 1 μM to 100 μM, about 1 μM to 10 μM, 10 μM to 100 μM, about 100 μM to 1000 μM or about 1 mM to 10 mM.

In some embodiments, the PF-06726304 is administered to a subject, for example to the middle ear at a concentration of about 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, or 10 mM.

In some embodiments, the EZH2 inhibitor is PF-06726304 and is administered systemically at a daily dose of about 50 mg to 5,000 mg/day, about 50 mg to 4000 mg/day, about 50 mg to 3000 mg/day, about 50 mg to 2000 mg/day, about 50 mg to 1000 mg/day, about 50 mg to 500 mg/day, about 100 mg to 2500 mg/day, about 100 mg to 2000 mg/day, about 100 mg to 1500 mg/day, about 100 mg to 1000 mg/day, about 100 mg to 500 mg/day, about 200 mg to 2500 mg/day, about 200 mg to 2000 mg/day, about 200 mg to 1600 mg/day, about 200 mg to 1000 mg/day, about 100 mg/day, about 200 mg/day, about 300 mg/day, about 400 mg/day, about 500 mg/day, about 600 mg/day, about 700 mg/day, about 800 mg/day, about 900 mg/day, about 1000 mg/day, about 1200 mg/day, about 1400 mg/day, about 1600 mg/day, about 1800 mg/day, or about 2000 mg/day.

In some embodiments, the EZH2 inhibitor is PF-06726304 and is administered to the subject at a concentration ratio of about 0.001 to 100 fold relative to an FDA approved concentration or about 0.01 to 50 fold relative to an FDA approved concentration or about 0.1 to 10 fold relative to an FDA approved concentration, or about 0.1 to 5 fold relative to an FDA approved, or about 1 to 5 fold relative to an FDA approved concentration.

In some embodiments, EZH2 inhibitor is PF-06726304 and is administered to the subject at about 0.01×. 0.1×, 1×, 2×, 3×, 4×, 5× or 10×, relative to an FDA approved dose. A PF-06726304 dose is for example the concentration liked on Table 7, column titled “Human Dosage”.

In some embodiments the additional epigenetic agent is a DOTL1 inhibitor.

In some embodiments, the DOT1L inhibitor is EPZ004777 and is administered for example to a cochlear cell in amount sufficient to achieve a concentration of about 0.01 nM to 1 mM, about 0.1 nM to 100 μM, about 1 nM to 100 μM, about 10 nM to 100 μM, about 1 nM to 10 nM, about 10 nM to 100 nM, about 100 nM to 1 μM, about 1 μM to 10 μM or about 10 μM to 100 μM, in the perilymph fluid in the inner ear.

In some embodiments, the EPZ004777 is administered, in amount sufficient to achieve a concentration of about 10 nM, 20 nM, 30 nM, 40 nM, 50 nM, 60 nM, 70 nM, 80 nM, 90 nM, 100 nM, 200 nM, 300 nM, 400 nM, 500 nM, 600 nM, 700 nM, 800 nM, 900 nM, 1 μM, 2 μM, 3 μM, 4 μM, 5 μM, 6 μM, 7 μM, 8 μM, 9 μM, 10 μM, 11 μM, 12 μM, 13 μM, 14 μM, 15 μM, 20 μM, 25 μM, 30 μM, 35 μM, 40 μM, 45 μM, or about 50 μM in the perilymph fluid in the inner ear.

In some embodiments, the DOT1L inhibitor is EPZ004777 is administered to a subject, for example to the middle ear at a concentration of about 0.01 μM to 1000 mM, about 0.1 μM to 100 mM, about 1 μM to 10 mM, about 10 μM to 1 mM, 10 μM to 100 μM, about 100 μM to 1000 μM, about 1 mM to 10 mM, or about 10 mM to 100 mM.

In some embodiments, the EPZ004777 is administered to a subject, for example to the middle ear at a concentration of about 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, 10 mM, 11 mM, 12 mM, 13 mM, 14 mM, 15 mM, 20 mM, 25 mM, 30 mM, 35 mM, 40 mM, 45 mM, or about 50 mM.

In some embodiments, the DOT1L inhibitor is EPZ004777 and is administered systemically at a daily dose of about 1-1000 mg/m2 per day IV, about 10-100 mg/m2 per day IV, about 10 mg/m2 per day IV, about 15 mg/m2 per day IV, about 20 mg/m2 per day IV, about 25 mg/m2 per day IV, about 30 mg/m2 per day IV, about 35 mg/m2 per day IV, about 40 mg/m2 per day IV, about 45 mg/m2 per day IV, about 50 mg/m2 per day IV, about 55 mg/m2 per day IV, about 60 mg/m2 per day IV, about 65 mg/m2 per day IV, about 70 mg/m2 per day IV, about 75 mg/m2 per day IV, about 80 mg/m2 per day IV, about 85 mg/m2 per day IV, about 90 mg/m2 per day IV, about 95 mg/m2 per day IV, about 100 mg/m2 per day IV, about 10 mg to 5,000 mg/day, about 10 mg to 3000 mg/day, about 10 mg to 1000 mg/day, about 10 mg to 500 mg/day, 20 mg to 5,000 mg/day, about 20 mg to 1000 mg/day, about 20 mg to 500 mg/day, about 10 mg/day, about 25 mg/day, about 50 mg/day, about 75 mg/day, about 100 mg/day, about 150 mg/day, about 200 mg/day, about 300 mg/day, about 400 mg/day, about 500 mg/day, about 600 mg/day, about 700 mg/day, about 800 mg/day, about 900 mg/day, or about 1000 mg/day.

In some embodiments, the DOT1L inhibitor is EPZ004777 and is administered to the subject at a concentration ratio of about 0.001 to 100 fold relative to an FDA approved concentration or about 0.01 to 50 fold relative to an FDA approved concentration or about 0.1 to 10 fold relative to an FDA approved concentration, or about 0.1 to 5 fold relative to an FDA approved, or about 1 to 5 fold relative to an FDA approved concentration.

In some embodiments, DOT1L inhibitor is EPZ004777 and is administered to the subject at about 0.01×. 0.1×, 1×, 2×, 3×, 4×, 5× or 10×, relative to an FDA approved dose. An EPZ004777 dose is for example the concentration listed on Table 8, column titled “Human Dosage”.

In some embodiments, the DOT1L inhibitor is SGC0946 and is administered for example to a cochlear cell in amount sufficient to achieve a concentration of about 0.01 nM to 1 mM, about 0.1 nM to 100 μM, about 1 nM to 100 μM, about 10 nM to 100 μM, about 1 nM to 10 nM, about 10 nM to 100 nM, about 100 nM to 1 μM, about 1 μM to 10 μM or about 10 μM to 100 μM, in the perilymph fluid in the inner ear.

In some embodiments, the SGC0946 is administered, in amount sufficient to achieve a concentration of about 10 nM, 20 nM, 30 nM, 40 nM, 50 nM, 60 nM, 70 nM, 80 nM, 90 nM, 100 nM, 200 nM, 300 nM, 400 nM, 500 nM, 600 nM, 700 nM, 800 nM, 900 nM, 1 μM, 2 μM, 3 μM, 4 μM, 5 μM, 6 μM, 7 μM, 8 μM, 9 μM, 10 μM, 11 μM, 12 μM, 13 μM, 14 μM, 15 μM, 20 μM, 25 μM, 30 μM, 35 μM, 40 μM, 45 μM, or about 50 μM in the perilymph fluid in the inner ear.

In some embodiments, the DOT1L inhibitor is SGC0946 is administered to a subject, for example to the middle ear at a concentration of about 0.01 μM to 1000 mM, about 0.1 μM to 100 mM, about 1 μM to 10 mM, about 10 μM to 1 mM, 10 μM to 100 μM, about 100 μM to 1000 μM, about 1 mM to 10 mM, or about 10 mM to 100 mM.

In some embodiments, the SGC0946 is administered to a subject, for example to the middle ear at a concentration of about 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, 10 mM, 11 mM, 12 mM, 13 mM, 14 mM, 15 mM, 20 mM, 25 mM, 30 mM, 35 mM, 40 mM, 45 mM, or about 50 mM.

In some embodiments, the DOT1L inhibitor is SGC0946 and is administered systemically at a daily dose of about 1-1000 mg/m2 per day IV, about 10-100 mg/m2 per day IV, about 10 mg/m2 per day IV, about 15 mg/m2 per day IV, about 20 mg/m2 per day IV, about 25 mg/m2 per day IV, about 30 mg/m2 per day IV, about 35 mg/m2 per day IV, about 40 mg/m2 per day IV, about 45 mg/m2 per day IV, about 50 mg/m2 per day IV, about 55 mg/m2 per day IV, about 60 mg/m2 per day IV, about 65 mg/m2 per day IV, about 70 mg/m2 per day IV, about 75 mg/m2 per day IV, about 80 mg/m2 per day IV, about 85 mg/m2 per day IV, about 90 mg/m2 per day IV, about 95 mg/m2 per day IV, about 100 mg/m2 per day IV, about 10 mg to 5,000 mg/day, about 10 mg to 3000 mg/day, about 10 mg to 1000 mg/day, about 10 mg to 500 mg/day, 20 mg to 5,000 mg/day, about 20 mg to 1000 mg/day, about 20 mg to 500 mg/day, about 10 mg/day, about 25 mg/day, about 50 mg/day, about 75 mg/day, about 100 mg/day, about 150 mg/day, about 200 mg/day, about 300 mg/day, about 400 mg/day, about 500 mg/day, about 600 mg/day, about 700 mg/day, about 800 mg/day, about 900 mg/day, or about 1000 mg/day.

In some embodiments, the DOT1L inhibitor is SGC0946 and is administered to the subject at a concentration ratio of about 0.001 to 100 fold relative to an FDA approved concentration or about 0.01 to 50 fold relative to an FDA approved concentration or about 0.1 to 10 fold relative to an FDA approved concentration, or about 0.1 to 5 fold relative to an FDA approved, or about 1 to 5 fold relative to an FDA approved concentration.

In some embodiments, DOT1L inhibitor is SGC0946 and is administered to the subject at about 0.01×. 0.1×, 1×, 2×, 3×, 4×, 5× or 10×, relative to an FDA approved dose. A SGC0946 dose is for example the concentration listed on Table 8, column titled “Human Dosage”.

In some embodiments, the DOT1L inhibitor is pinometostat and is administered for example to a cochlear cell in amount sufficient to achieve a concentration of about 0.01 nM to 1 mM, about 0.1 nM to 100 μM, about 1 nM to 100 μM, about 10 nM to 100 μM, about 1 nM to 10 nM, about 10 nM to 100 nM, about 100 nM to 1 μM, about 1 μM to 10 μM or about 10 μM to 100 μM, in the perilymph fluid in the inner ear.

In some embodiments, the pinometostat is administered, in amount sufficient to achieve a concentration of about 10 nM, 20 nM, 30 nM, 40 nM, 50 nM, 60 nM, 70 nM, 80 nM, 90 nM, 100 nM, 200 nM, 300 nM, 400 nM, 500 nM, 600 nM, 700 nM, 800 nM, 900 nM, 1 μM, 2 μM, 3 μM, 4 μM, 5 μM, 6 μM, 7 μM, 8 μM, 9 μM, 10 μM, 11 μM, 12 μM, 13 μM, 14 μM, 15 μM, 20 μM, 25 μM, 30 μM, 35 μM, 40 μM, 45 μM, or about 50 μM in the perilymph fluid in the inner ear.

In some embodiments, the DOT1L inhibitor is pinometostat is administered to a subject, for example to the middle ear at a concentration of about 0.01 μM to 1000 mM, about 0.1 μM to 100 mM, about 1 μM to 10 mM, about 10 μM to 1 mM, 10 μM to 100 μM, about 100 μM to 1000 μM, about 1 mM to 10 mM, or about 10 mM to 100 mM.

In some embodiments, the pinometostat is administered to a subject, for example to the middle ear at a concentration of about 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, 10 mM, 11 mM, 12 mM, 13 mM, 14 mM, 15 mM, 20 mM, 25 mM, 30 mM, 35 mM, 40 mM, 45 mM, or about 50 mM.

In some embodiments, the DOT1L inhibitor is pinometostat and is administered systemically at a daily dose of about 1-1000 mg/m2 per day IV, about 10-100 mg/m2 per day IV, about 10 mg/m2 per day IV, about 15 mg/m2 per day IV, about 20 mg/m2 per day IV, about 25 mg/m2 per day IV, about 30 mg/m2 per day IV, about 35 mg/m2 per day IV, about 40 mg/m2 per day IV, about 45 mg/m2 per day IV, about 50 mg/m2 per day IV, about 55 mg/m2 per day IV, about 60 mg/m2 per day IV, about 65 mg/m2 per day IV, about 70 mg/m2 per day IV, about 75 mg/m2 per day IV, about 80 mg/m2 per day IV, about 85 mg/m2 per day IV, about 90 mg/m2 per day IV, about 95 mg/m2 per day IV, about 100 mg/m2 per day IV, about 10 mg to 5,000 mg/day, about 10 mg to 3000 mg/day, about 10 mg to 1000 mg/day, about 10 mg to 500 mg/day, 20 mg to 5,000 mg/day, about 20 mg to 1000 mg/day, about 20 mg to 500 mg/day, about 10 mg/day, about 25 mg/day, about 50 mg/day, about 75 mg/day, about 100 mg/day, about 150 mg/day, about 200 mg/day, about 300 mg/day, about 400 mg/day, about 500 mg/day, about 600 mg/day, about 700 mg/day, about 800 mg/day, about 900 mg/day, or about 1000 mg/day.

In some embodiments, the DOT1L inhibitor is pinometostat and is administered to the subject at a concentration ratio of about 0.001 to 100 fold relative to an FDA approved concentration or about 0.01 to 50 fold relative to an FDA approved concentration or about 0.1 to 10 fold relative to an FDA approved concentration, or about 0.1 to 5 fold relative to an FDA approved, or about 1 to 5 fold relative to an FDA approved concentration.

In some embodiments, DOT1L inhibitor is pinometostat and is administered to the subject at about 0.01×. 0.1×, 1×, 2×, 3×, 4×, 5× or 10×, relative to an FDA approved dose. A pinometostat dose is for example the concentration listed on Table 8, column titled “Human Dosage”.

In some embodiments the additional epigenetic agent is a KDM inhibitor.

In some embodiments, the KDM inhibitor is AS 8351 and is administered for example to a cochlear cell in amount sufficient to achieve a concentration of about 0.01 nM to 1 mM, about 0.1 nM to 100 μM, about 1 nM to 10 μM, about 10 nM to 10 μM, about 1 nM to 10 nM, about 10 nM to 100 nM, 100 nM to 1 μM, or about 1 μM to 10 μM, in the perilymph fluid in the inner ear.

In some embodiments, the AS 8351 is administered, in amount sufficient to achieve a concentration of about 10 nM, 20 nM, 30 nM, 40 nM, 50 nM, 60 nM, 70 nM, 80 nM, 90 nM, 100 nM, 200 nM, 300 nM, 400 nM, 500 nM, 600 nM, 700 nM, 800 nM, 900 nM, 1 μM, 2 μM, 3 μM, 4 μM, 5 μM, 6 μM, 7 μM, 8 μM, 9 μM, or about 10 μM, in the perilymph fluid in the inner ear.

In some embodiments, the KDM inhibitor is AS 8351 is administered to a subject, for example to the middle ear at a concentration of about 0.01 μM to 1000 mM, about 0.1 μM to 100 mM, about 1 μM to 10 mM, about 10 μM to 1000 μM, about 1 μM to 10 μM, 10 μM to 100 μM, about 100 μM to 1000 μM or about 1 mM to 10 mM.

In some embodiments, the AS 8351 is administered to a subject, for example to the middle ear at a concentration of about 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, or 10 mM.

In some embodiments, the KDM inhibitor is AS 8351 and is administered systemically at a daily dose of about 50 mg to 5,000 mg/day, about 50 mg to 4000 mg/day, about 50 mg to 3000 mg/day, about 50 mg to 2000 mg/day, about 50 mg to 1000 mg/day, about 50 mg to 500 mg/day, about 100 mg to 2500 mg/day, about 100 mg to 2000 mg/day, about 100 mg to 1500 mg/day, about 100 mg to 1000 mg/day, about 100 mg to 500 mg/day, about 200 mg to 2500 mg/day, about 200 mg to 2000 mg/day, about 200 mg to 1600 mg/day, about 200 mg to 1000 mg/day, about 100 mg/day, about 200 mg/day, about 300 mg/day, about 400 mg/day, about 500 mg/day, about 600 mg/day, about 700 mg/day, about 800 mg/day, about 900 mg/day, about 1000 mg/day, about 1200 mg/day, about 1400 mg/day, about 1600 mg/day, about 1800 mg/day, or about 2000 mg/day.

In some embodiments, the KDM inhibitor is AS 8351 and is administered to the subject at a concentration ratio of about 0.001 to 100 fold relative to an FDA approved concentration or about 0.01 to 50 fold relative to an FDA approved concentration or about 0.1 to 10 fold relative to an FDA approved concentration, or about 0.1 to 5 fold relative to an FDA approved, or about 1 to 5 fold relative to an FDA approved concentration.

In some embodiments, KDM inhibitor is AS 8351 and is administered to the subject at about 0.01×. 0.1×, 1×, 2×, 3×, 4×, 5× or 10×, relative to an FDA approved dose. An AS 8351 dose is for example the concentration listed on Table 9 column titled “Human Dosage”.

In some embodiments, the KDM inhibitor is TC-E 5002 and is administered for example to a cochlear cell in amount sufficient to achieve a concentration of about 0.01 nM to 1 mM, about 0.1 nM to 100 μM, about 1 nM to 10 μM, about 10 nM to 10 μM, about 1 nM to 10 nM, about 10 nM to 100 nM, 100 nM to 1 μM, or about 1 μM to 10 μM, in the perilymph fluid in the inner ear.

In some embodiments, the TC-E 5002 is administered, in amount sufficient to achieve a concentration of about 10 nM, 50 nM, 75 nM, 100 nM, 110 nM, 120 nM, 130 nM, 140 nM, 150 nM, 160 nM, 170 nM, 180 nM, 190 nM, 200 nM, 300 nM, 400 nM, 500 nM, 600 nM, 700 nM, 800 nM, 900 nM, 1 μM, 2 μM, 3 μM, 4 μM, 5 μM, 6 μM, 7 μM, 8 μM, 9 μM, or about 10 μM, in the perilymph fluid in the inner ear.

In some embodiments, the KDM inhibitor is TC-E 5002 is administered to a subject, for example to the middle ear at a concentration of about 0.01 μM to 1000 mM, about 0.1 μM to 100 mM, about 1 μM to 10 mM, about 10 μM to 1000 μM, about 1 μM to 10 μM, 10 μM to 100 μM, about 100 μM to 1000 μM or about 1 mM to 10 mM.

In some embodiments, the AS TC-E 5002 is administered to a subject, for example to the middle ear at a concentration of about 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, or 10 mM.

In some embodiments, the KDM inhibitor is TC-E 5002 and is administered systemically at a daily dose of about 50 mg to 5,000 mg/day, about 50 mg to 4000 mg/day, about 50 mg to 3000 mg/day, about 50 mg to 2000 mg/day, about 50 mg to 1000 mg/day, about 50 mg to 500 mg/day, about 100 mg to 2500 mg/day, about 100 mg to 2000 mg/day, about 100 mg to 1500 mg/day, about 100 mg to 1000 mg/day, about 100 mg to 500 mg/day, about 200 mg to 2500 mg/day, about 200 mg to 2000 mg/day, about 200 mg to 1600 mg/day, about 200 mg to 1000 mg/day, about 100 mg/day, about 200 mg/day, about 300 mg/day, about 400 mg/day, about 500 mg/day, about 600 mg/day, about 700 mg/day, about 800 mg/day, about 900 mg/day, about 1000 mg/day, about 1200 mg/day, about 1400 mg/day, about 1600 mg/day, about 1800 mg/day, or about 2000 mg/day.

In some embodiments, the KDM inhibitor is AS TC-E 5002 and is administered to the subject at a concentration ratio of about 0.001 to 100 fold relative to an FDA approved concentration or about 0.01 to 50 fold relative to an FDA approved concentration or about 0.1 to 10 fold relative to an FDA approved concentration, or about 0.1 to 5 fold relative to an FDA approved, or about 1 to 5 fold relative to an FDA approved concentration.

In some embodiments, KDM inhibitor is TC-E 5002 and is administered to the subject at about 0.01×. 0.1×, 1×, 2×, 3×, 4×, 5× or 10×, relative to an FDA approved dose. An TC-E 5002 dose is for example the concentration listed on Table 9, column titled “Human Dosage”.

In some embodiments, the KDM inhibitor is EPT-10318 and is administered for example to a cochlear cell in amount sufficient to achieve a concentration of about 0.001 nM to 100 μM, about 0.01 nM to 10 μM, about 0.1 nM to 1 μM, about 1 nM to 100 nM, about 1 nM to 10 nM, about 10 nM to 100 nM, or about 100 nM to 1 μM, in the perilymph fluid in the inner ear

In some embodiments, the EPT-40318 is administered, in amount sufficient to achieve a concentration of about 0.1 nM, 0.2 nM, 0.3 nM, 0.4 nM, 0.5 nM, 0.6 nM, 0.7 nM, 0.8 nM, 0.9 nM, 1.0 nM, 2.0 nM, 3.0 nM, 4.0 nM, 5.0 nM, 6.0 nM, 7.0 nM, 8.0 nM, 9.0 nM, 10 nM, 20 nM, 30 nM, 40 nM, 50 nM, 60 nM, 70 nM, 80 nM, 90 nM, 100 nM, 200 nM, 300 nM, 400 nM, 500 nM, 600 nM, 700 nM, 800 nM, 900 nM, or about 1 μM in the perilymph fluid in the inner ear.

In some embodiments, the KDM inhibitor is EPT-10318 is administered to a subject, for example to the middle ear at a concentration of 0.001 μM to 100 mM, about 0.01 μM to 10 mM, about 0.1 μM to 1 mM, about 1 μM to 100 μM, about 1 μM to 10 μM, 10 μM to 100 μM, or about 100 μM to 1 mM.

In some embodiments, the EPT-10318 is administered to a subject, for example to the middle ear at a concentration of about 0.1 μM, 0.2. μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, or about 1 mM.

In some embodiments, the KDM inhibitor is EPT-10318 and is administered systemically at a daily dose of about 50 mg to 5,000 mg/day, about 50 mg to 4000 mg/day, about 50 mg to 3000 mg/day, about 50 mg to 2000 mg/day, about 50 mg to 1000 mg/day, about 50 mg to 500 mg/day, about 100 mg to 2500 mg/day, about 100 mg to 2000 mg/day, about 100 mg to 1500 mg/day, about 100 mg to 1000 mg/day, about 100 mg to 500 mg/day, about 150 mg to 2500 mg/day, about 150 mg to 2000 mg/day, about 150 mg to 1500 mg/day, about 150 mg to 1250 mg/day, about 75 mg/day, about 100 mg/day, about 150 mg/day, about 200 mg/day, about 300 mg/day, about 400 mg/day, about 500 mg/day, about 600 mg/day, about 700 mg/day, about 800 mg/day, about 900 mg/day, about 1000 mg/day, about 1200 mg/day, about 1400 mg/day, about 1600 mg/day, about 1800 mg/day, or about 2000 mg/day.

In some embodiments, the KDM inhibitor is EPT-103182 and is administered to the subject at a concentration ratio of about 0.001 to 100 fold relative to an FDA approved concentration or about 0.01 to 50 fold relative to an FDA approved concentration or about 0.1 to 10 fold relative to an FDA approved concentration, or about 0.1 to 5 fold relative to an FDA approved, or about 1 to 5 fold relative to an FDA approved concentration. In some embodiments, KDM inhibitor is EPT-103182 and is administered to the subject at about 0.01×. 0.1×, 1×, 2×, 3×, 4×, 5× or 10×, relative to an FDA approved dose. An EPT-103182 dose is for example the concentration listed on Table 9, column titled “Human Dosage”.

In some embodiments the EZH2 inhibitor is CPI-1205 and the Wnt agonist is AZD1080. In some embodiments, CPI-1205 is administered, in amount sufficient to achieve a concentration of about 0.1 nM, 0.2 nM, 0.3 nM, 0.4 nM, 0.5 nM, 0.6 nM, 0.7 nM, 0.8 nM, 0.9 nM, 1.0 nM, 2.0 nM, 3.0 nM, 4.0 nM, 5.0 nM, 6.0 nM, 7.0 nM, 8.0 nM, 9.0 nM, 10 nM, 20 nM, 30 nM, 40 nM, 50 nM, 60 nM, 70 nM, 80 nM, 90 nM, 100 nM, 200 nM, 300 nM, 400 nM, 500 nM, 600 nM, 700 nM, 800 nM, 900 nM, 1 μM, 2 μM, 3 μM, 4 μM, 5 μM, 6 μM, 7 μM, 8 μM, 9 μM, 10 μM, 12 μM, 14 μM, 16 μM, 18 μM, 20 μM, 25 μM, about 30 μM in the perilymph fluid in the inner ear and AZD1080 is administered, in amount sufficient to achieve a concentration of about is about 1 μM, 2 μM, 3 μM, 4 μM, 5 μM, 6 μM, 7 μM, 8 μM, 9 μM, or 10 μM, in the perilymph fluid in the inner ear. Alternatively, the CPI-1205 is administered to a subject, for example to the middle ear at a concentration of about 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, 10 mM, 12 mM, 14 mM, 16 mM, 18 mM, 20 mM, 25 mM or about 30 mM and AZD1080, and is administered to a subject, for example to the middle ear at a concentration of about 1 mM, 2 mM, 3 mM, 4 mM, 5 mM, 6 mM, 7 mM, 8 mM, 9 mM, or 10 mM.

In some embodiments the EZH2 inhibitor is CPI-1205 and the Wnt agonist is LY2090314. In some embodiments, CPI-1205 is administered, in amount sufficient to achieve a concentration of about 0.1 nM, 0.2 nM, 0.3 nM, 0.4 nM, 0.5 nM, 0.6 nM, 0.7 nM, 0.8 nM, 0.9 nM, 1.0 nM, 2.0 nM, 3.0 nM, 4.0 nM, 5.0 nM, 6.0 nM, 7.0 nM, 8.0 nM, 9.0 nM, 10 nM, 2.0 nM, 30 nM, 40 nM, 50 nM, 60 nM, 70 nM, 80 nM, 90 nM, 100 nM, 200 nM, 300 nM, 400 nM, 500 nM, 600 nM, 700 nM, 800 nM, 900 nM, 1 μM, 2 μM, 3 μM, 4 μM, 5 μM, 6 μM, 7 μM, 8 μM, 9 μM, 10 μM, 12 μM, 14 μM, 16 μM, 18 μM, 20 μM, 25 μM, of about 30 μM in the perilymph fluid in the inner ear and LY2090314 is administered, in amount sufficient to achieve a concentration of about 1 nM, 5 nM, 10 nM, 15 nM, 20 nM, or 40 nM in the perilymph fluid in the inner ear. Alternatively, the CPI-1205 is administered to a subject, for example to the middle ear at a concentration of about 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, 10 mM, 12 mM, 14 mM, 16 mM, 18 mM, 20 mM, 25 mM or about 30 mM and LY2090314, and is administered to a subject, for example to the middle ear at a concentration of about 1 μM, 5 μM, 10 μM, 15 μM, 20 μM, or 40 μM.

In some embodiments the EZH2 inhibitor is CPI-1205 and the Wnt agonist is a substituted 3-imidazo[1,2-a]pyridin-3-yl-4-(1,2,3,4-tetrahydro-[1,4]diazepino-[6,7,1-hi]indol-7-yl)pyrrole-2,5-dione. In some embodiments, CPI-1205 is administered, in amount sufficient to achieve a concentration of about 0.1 nM, 0.2 nM, 0.3 nM, 0.4 nM, 0.5 nM, 0.6 nM, 0.7 nM, 0.8 nM, 0.9 nM, 1.0 nM, 2.0 nM, 3.0 nM, 4.0 nM, 5.0 nM, 6.0 nM, 7.0 nM, 8.0 nM, 9.0 nM, 10 nM, 20 nM, 30 nM, 40 nM, 50 nM, 60 nM, 70 nM, 80 nM, 90 nM, 100 nM, 200 nM, 300 nM, 400 nM, 500 nM, 600 nM, 700 nM, 800 nM, 900 nM, 1 μM, 2 μM, 3 μM, 4 μM, 5 μM, 6 μM, 7 μM, 8 μM, 9 μM, 10 μM, 12 μM, 14 μM, 16 μM, 18 μM, 20 μM, 25 μM, or about 30 μM in the perilymph fluid in the inner ear and the substituted 3-Imidazo[1,2-a]pyridin-3-yl-4-(1,2,3,4-tetrahydro-[1,4]diazepino-[6,7,1-hi]indol-7-yl)pyrrole-2,5-dione is administered, in amount sufficient to achieve a concentration of about 1 nM, 5 nM, 10 nM, 15 nM, 20 nM, 50 nM, 100 nM, 250 nM, or 500 nM, in the perilymph fluid in the inner ear. Alternatively, the CPI-1205 is administered to a subject, for example to the middle ear at a concentration of about 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, 10 mM, 12 mM, 14 mM, 16 mM, 18 mM, 20 mM, 25 mM or about 30 mM and the substituted 3-Imidazo[1,2-a]pyridin-3-yl-4-(1,2,3,4-tetrahydro-[1,4]diazepino-[6,7,1-hi]indol-7-yl)pyrrole-2,5-dione and is administered to a subject, for example to the middle ear at a concentration of about 1 μM, 5 μM, 10 μM, 15 μM, 20 μM, 50 μM, 100 μM, 250 μM, or 500 μM.

In some embodiments the EZH2 inhibitor is CPI-1205 and the Wnt agonist is GSK3 inhibitor XXII. In some embodiments, CPI-1205 is administered, in amount sufficient to achieve a concentration of about 0.1 nM, 0.2 nM, 0.3 nM, 0.4 nM, 0.5 nM, 0.6 nM, 0.7 nM, 0.8 nM, 0.9 nM, 1.0 nM, 2.0 nM, 3.0 nM, 4.0 nM, 5.0 nM, 6.0 nM, 7.0 nM, 8.0 nM, 9.0 nM, 10 nM, 20 nM, 30 nM, 40 nM, 50 nM, 60 nM, 70 nM, 80 nM, 90 nM, 100 nM, 200 nM, 300 nM, 400 nM, 500 nM, 600 nM, 700 nM, 800 nM, 900 nM, 1 μM, 2 μM, 3 μM, 4 μM, 5 μM, 6 μM, 7 μM, 8 μM, 9 μM, 10 μM, 12 μM, 14 μM, 16 μM, 18 μM, 20 μM, 25 μM, or about 30 μM in the perilymph fluid in the inner ear and GSK3-inhibitor XXII is administered, in amount sufficient to achieve a concentration of about 0.1 μM, 0.2 μM, 0.3 μM, 0.4 μM, 0.5 μM, 0.6 μM, 0.7 μM, 0.8 μM, 0.9 μM, or 1.0 μM, in the perilymph fluid in the inner ear. Alternatively, the CPI-1205 is administered to a subject, for example to the middle ear at a concentration of about 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, 10 mM, 12 mM, 14 mM, 16 mM, 18 mM, 20 mM, 25 mM or about 30 mM and the GSK3-inhibitor XXII is administered, in amount sufficient to achieve a concentration of about 0.1 mM, 0.2 mM, 0.3 mM, 0.4 mM, 0.5 mM, 0.6 mM, 0.7 mM, 0.8 mM, 0.9 mM, or 1.0 mM, in the perilymph fluid in the inner ear.

In some embodiments the EZH2 inhibitor is CPI-1205 and the Wnt agonist is CHIR99021. In some embodiments, CPI-1205 is administered, in amount sufficient to achieve a concentration of about 0.1 nM, 0.2 nM, 0.3 nM, 0.4 nM, 0.5 nM, 0.6 nM, 0.7 nM, 0.8 nM, 0.9 nM, 1.0 nM, 2.0 nM, 3.0 nM, 4.0 nM, 5.0 nM, 6.0 nM, 7.0 nM, 8.0 nM, 9.0 nM, 10 nM, 20 nM, 30 nM, 40 nM, 50 nM, 60 nM, 70 nM, 80 nM, 90 nM, 100 nM, 200 nM, 300 nM, 400 nM, 500 nM, 600 nM, 700 nM, 800 nM, 900 nM, 1 μM, 2 μM, 3 μM, 4 μM, 5 μM, 6 μM, 7 μM, 8 μM, 9 μM, 10 μM, 12 μM, 14 μM, 16 μM, 18 μM, 20 μM, 25 μM, or about 30 μM in the perilymph fluid in the inner ear and CHIR99021 is administered, in amount sufficient to achieve a concentration of about 1 μM, 2 μM, 3 μM, 4 μM, 5 μM, 6 μM, 7 μM, 8 μM, 9 μM, or 10 μM, in the perilymph fluid in the inner ear Alternatively, the CPI-1205 is administered to a subject, for example to the middle ear at a concentration of about 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, 10 mM, 12 mM, 14 mM, 16 mM, 18 mM, 20 mM, 25 mM, or about 30 mM and CHIR99021 is administered to a subject, for example to the middle ear at a concentration of about 1 mM, 2 mM, 3 mM, 4 mM, 5 mM, 6 mM, 7 mM, 8 mM, 9 mM, or 10 mM.

In some embodiments the EZH2 inhibitor is CPI-169 and the Wnt agonist is AZD1080. In some embodiments, CPI-169 is administered, in amount sufficient to achieve a concentration of about 0.1 nM, 0.2 nM, 0.3 nM, 0.4 nM, 0.5 nM, 0.6 nM, 0.7 nM, 0.8 nM, 0.9 nM, 1.0 nM, 2.0 nM, 3.0 nM, 4.0 nM, 5.0 nM, 6.0 nM, 7.0 nM, 8.0 nM, 9.0 nM, 10 nM, 20 nM, 30 nM, 40 nM, 50 nM, 60 nM, 70 nM, 80 nM, 90 nM, 100 nM, 200 nM, 300 nM, 400 nM, 500 nM, 1 μM, 5 μM, 10 μM, or 50 μM in the perilymph fluid in the inner ear and AZD1080 is administered, in amount sufficient to achieve a concentration of about is about 1 μM, 2 μM, 3 μM, 4 μM, 5 μM, 6 μM, 7 μM, 8 μM, 9 μM, or 10 μM, in the perilymph fluid in the inner ear. Alternatively, the CPI-169 is administered to a subject, for example to the middle ear at a concentration of about 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 1 mM, 5 mM, 10 mM, or 50 μM and AZD1080, and is administered to a subject, for example to the middle ear at a concentration of about 1 mM, 2 mM, 3 mM, 4 mM, 5 mM, 6 mM, 7 mM, 8 mM, 9 mM, or 10 mM.

In some embodiments the EZH2 inhibitor is CPI-169 and the Wnt agonist is LY2090314 In some embodiments, CPI-169 is administered, in amount sufficient to achieve a concentration of about 0.1 nM, 0.2 nM, 0.3 nM, 0.4 nM, 0.5 nM, 0.6 nM, 0.7 nM, 0.8 nM, 0.9 nM, 1.0 nM, 2.0 nM, 3.0 nM, 4.0 nM, 5.0 nM, 6.0 nM, 7.0 nM, 8.0 nM, 9.0 nM, 10 nM, 20 nM, 30 nM, 40 nM, 50 nM, 60 nM, 70 nM, 80 nM, 90 nM, 100 nM, 200 nM, 300 nM, 400 nM, 500 nM, 1 μM, 5 μM, 10 μM, or 50 μM in the perilymph fluid in the inner ear and LY2090314 is administered, in amount sufficient to achieve a concentration of about 1 nM, 5 nM, 10 nM, 15 nM, 20 nM or 40 nM, in the perilymph fluid in the inner ear. Alternatively, the CPI-169 is administered to a subject, for example to the middle ear at a concentration of about 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 1 mM, 5 mM, 10 mM, or 50 mM and LY2090314, and is administered to a subject, for example to the middle ear at a concentration of about 1 μM, 5 μM, 10 μM, 15 μM, 20 μM, or 40 μM.

In some embodiments the EZH2 inhibitor is CPI-169 and the Wnt agonist is a substituted 3-Imidazo[1,2-a]pyridin-3-yl-4-(1,2,3,4-tetrahydro-[1,4]diazepino-[6,7,1-hi]indol-7-yl)pyrrole-2,5-dione. In some embodiments, CPI-169 is administered, in amount sufficient to achieve a concentration of about 0.1 nM, 0.2 nM, 0.3 nM, 0.4 nM, 0.5 nM, 0.6 nM, 0.7 nM, 0.8 nM, 0.9 nM, 1.0 nM, 2.0 nM, 3.0 nM, 4.0 nM, 5.0 nM, 6.0 nM, 7.0 nM, 8.0 nM, 9.0 nM, 10 nM, 20 nM, 30 nM, 40 nM, 50 nM, 60 nM, 70 nM, 80 nM, 90 nM, 100 nM, 200 nM, 300 nM, 400 nM, 500 nM, 1 μM, 5 μM, 10 μM, or 50 μM in the perilymph fluid in the inner ear and the substituted 3-Imidazo[1,2-a]pyridin-3-yl-4-(1,2,3,4-tetrahydro-[1,4]diazepino-[6,7,1-hi]indol-7-yl)pyrrole-2,5-dione is administered, in amount sufficient to achieve a concentration of about 1 nM, 5 nM, 10 nM, 15 nM, 20 nM 50 nM, 100 nM, 250 nM, or 500 nM, in the perilymph fluid in the inner ear. Alternatively, the CPI-169 is administered to a subject, for example to the middle ear at a concentration of about 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 1 mM, 5 mM, 10 mM, or 50 mM and the substituted 3-Imidazo[1,2-a]pyridin-3-yl-4-(1,2,3,4-tetrahydro-[1,4]diazepino-[6,7,1-hi]indol-7-yl)pyrrole-2,5-dione and is administered to a subject, for example to the middle ear at a concentration of about 1 μM, 5 μM, 10 μM, 15 μM, 20 μM, 50 μM, 100 μM, 250 μM, or 500 μM.

In some embodiments the EZH2 inhibitor is CPI-169 and the Wnt agonist is GSK3 inhibitor XXII. In some embodiments, CPI-169 is administered, in amount sufficient to achieve a concentration of about 0.1 nM, 0.2 nM, 0.3 nM, 0.4 nM, 0.5 nM, 0.6 nM, 0.7 nM, 0.8 nM, 0.9 nM, 1.0 nM, 2.0 nM, 3.0 nM, 4.0 nM, 5.0 nM, 6.0 nM, 7.0 nM, 8.0 nM, 9.0 nM, 10 nM, 20 mM, 30 nM, 40 nM, 50 nM, 60 nM, 70 nM, 80 nM, 90 nM, 100 nM, 200 nM, 300 nM, 400 nM, 500 nM, 1 μM, 5 μM, 10 μM, or 50 μM in the perilymph fluid in the inner ear and GSK3-inhibitor XXII is administered, in amount sufficient to achieve a concentration of about 0.1 μM, 0.2 μM, 0.3 μM, 0.4 μM, 0.5 μM, 0.6 μM, 0.7 μM, 0.8 μM, 0.9 μM, or 1.0 μM, in the perilymph fluid in the inner ear. Alternatively, the CPI-169 is administered to a subject, for example to the middle ear at a concentration of about 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 1 mM, 5 mM, 10 mM, or 50 mM, the GSK3-inhibitor XXII is administered, in amount sufficient to achieve a concentration of about 0.1 mM, 0.2 mM, 0.3 mM, 0.4 mM, 0.5 mM, 0.6 mM, 0.7 mM, 0.8 mM, 0.9 mM, or 1.0 mM, in the perilymph fluid in the inner ear.

In some embodiments the EZH2 inhibitor is CPI-169 and the Wnt agonist is CHIR99021. In some embodiments, CPI-169 is administered, in amount sufficient to achieve a concentration of about 0.1 nM, 0.2 nM, 0.3 nM, 0.4 nM, 0.5 nM, 0.6 nM, 0.7 nM, 0.8 nM, 0.9 nM, 1.0 nM, 2.0 nM, 3.0 nM, 4.0 nM, 5.0 nM, 6.0 nM, 7.0 nM, 8.0 nM, 9.0 nM, 10 nM, 20 mM, 30 nM, 40 nM, 50 nM, 60 nM, 70 nM, 80 nM, 90 nM, 100 nM, 200 nM, 300 nM, 400 nM, 500 nM, 1 μM, 5 μM, 10 μM, or 50 μM in the perilymph fluid in the inner ear and CHIR99021 is administered, in amount sufficient to achieve a concentration of about 1 μ, 2 μM, 3 μM, 4 μM, 5 μM, 6 μM, 7 μM, 8 μM, 9 μM, or 10 μM, in the perilymph fluid in the inner ear Alternatively, the CPI-169 is administered to a subject, for example to the middle ear at a concentration of about 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 1 mM, 5 mM, 10 mM, or 50 mM and CHIR99021 is administered to a subject, for example to the middle ear at a concentration of about 1 mM, 2 mM, 3 mM, 4 mM, 5 mM, 6 mM, 7 mM, 8 mM, 9 mM, or 10 mM.

In some embodiments the EZH2 inhibitor is tazemetostat and the Wnt agonist is AZD1080. In some embodiments, tazemetostat is administered, in amount sufficient to achieve a concentration of about 0.1 nM, 0.2 nM, 0.3 nM, 0.4 nM, 0.5 nM, 0.6 nM, 0.7 nM, 0.8 nM, 0.9 nM, 1.0 nM, 2.0 nM, 3.0 nM, 4.0 nM, 5.0 nM, 6.0 nM, 7.0 nM, 8.0 nM, 9.0 nM, 10 nM, 20 nM, 30 nM, 40 nM, 50 nM, 60 nM, 70 nM, 80 nM, 90 nM, 100 nM, 200 mM, 300 nM, 400 nM, 500 nM, 1 μM, 5 μM, 10 μM, or 50 μM in the perilymph fluid in the inner ear and AZD1080 is administered, in amount sufficient to achieve a concentration of about is about 1 μM, 2 μM, 3 μM, 4 μM, 5 μM, 6 μM, 7 μM, 8 μM, 9 μM, or 10 μM, in the perilymph fluid in the inner ear. Alternatively, the tazemetostat is administered to a subject, for example to the middle ear at a concentration of about 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 1 mM, 5 mM, 10 mM, or 50 μM and AZD1080, and is administered to a subject, for example to the middle ear at a concentration of about 1 mM, 2 mM, 3 mM, 4 mM, 5 mM, 6 mM, 7 mM, 8 mM, 9 mM, or 10 mM.

In some embodiments the EZH2 inhibitor is tazemetostat and the Wnt agonist is LY2090314 In some embodiments, tazemetostat is administered, in amount sufficient to achieve a concentration of about 0.1 nM, 0.2 nM, 0.3 nM, 0.4 nM, 0.5 nM, 0.6 nM, 0.7 nM, 0.8 nM, 0.9 nM, 1.0 nM, 2.0 nM, 3.0 nM, 4.0 nM, 5.0 nM, 6.0 nM, 7.0 nM, 8.0 nM, 9.0 nM, 10 nM, 20 nM, 30 nM, 40 nM, 50 nM, 60 nM, 70 nM, 80 nM, 90 nM, 100 nM, 200 nM, 300 nM, 400 nM, 500 nM, 1 μM, 5 μM, 10 μM, or 50 μM in the perilymph fluid in the inner ear and LY2090314 is administered, in amount sufficient to achieve a concentration of about 1 nM, 5 nM, 10 nM, 15 nM, 20 nM or 40 nM, in the perilymph fluid in the inner ear. Alternatively, the tazemetostat is administered to a subject, for example to the middle ear at a concentration of about 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 1 mM, 5 mM, 10 mM, or 50 mM and LY2090314, and is administered to a subject, for example to the middle ear at a concentration of about 1 μM, 5 μM, 10 μM, 15 μM, 20 μM, or 40 μM.

In some embodiments the EZH2 inhibitor is tazemetostat and the Wnt agonist is a substituted 3-Imidazo[1,2-a]pyridin-3-yl-4-(1,2,3,4-tetrahydro-[1,4]diazepino-[6,7,1-hi]indol-7-yl)pyrrole-2,5-dione. In some embodiments, tazemetostat is administered, in amount sufficient to achieve a concentration of about 0.1 nM, 0.2 nM, 0.3 nM, 0.4 nM, 0.5 nM, 0.6 nM, 0.7 nM, 0.8 nM, 0.9 nM, 1.0 nM, 2.0 nM, 3.0 nM, 4.0 nM, 5.0 nM, 6.0 nM, 7.0 nM, 8.0 nM, 9.0 nM, 10 nM, 20 nM, 30 nM, 40 nM, 50 nM, 60 nM, 70 nM, 80 nM, 90 nM, 100 nM, 200 nM, 300 nM, 400 nM, 500 nM, 1 μM, 5 μM, 10 μM, or 50 μM in the perilymph fluid in the inner ear and the substituted 3-Imidazo[1,2-a]pyridin-3-yl-4-(1,2,3,4-tetrahydro-[1,4]diazepino-[6,7,1-hi]indol-7-yl)pyrrole-2,5-dione is administered, in amount sufficient to achieve a concentration of about 1 nM, 5 nM, 10 nM, 15 nM, 20 nM 50 nM, 100 nM, 250 nM, or 500 nM, in the perilymph fluid in the inner ear. Alternatively, the tazemetostat is administered to a subject, for example to the middle ear at a concentration of about 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 1 mM, 5 mM, 10 mM, or 50 mM and the substituted 3-Imidazo[1,2-a]pyridin-3-yl-4-(1,2,3,4-tetrahydro-[1,4]diazepino-[6,7,1-hi]indol-7-yl)pyrrole-2,5-dione and is administered to a subject, for example to the middle ear at a concentration of about 1 μM, 5 μM, 10 μM, 15 μM, 20 μM, 50 μM, 100 μM, 250 μM, or 500 μM.

In some embodiments the EZH2 inhibitor is tazemetostat and the Wnt agonist is GSK3 inhibitor XXII. In some embodiments, tazemetostat is administered, in amount sufficient to achieve a concentration of about 0.1 nM, 0.2 nM, 0.3 nM, 0.4 nM, 0.5 nM, 0.6 nM, 0.7 nM, 0.8 nM, 0.9 nM, 1.0 nM, 2.0 nM, 3.0 nM, 4.0 nM, 5.0 nM, 6.0 nM, 7.0 nM, 8.0 nM, 9.0 nM, 10 nM, 20 nM, 30 nM, 40 nM, 50 nM, 60 nM, 70 nM, 80 nM, 90 nM, 100 nM, 200 nM, 300 nM, 400 nM, 500 nM, 1 μM, 5 μM, 10 μM, or 50 μM in the perilymph fluid in the inner ear and GSK3-inhibitor XXII is administered, in amount sufficient to achieve a concentration of about 0.1 μM, 0.2 μM, 0.3 μM, 0.4 μM, 0.5 μM, 0.6 μM, 0.7 μM, 0.8 μM, 0.9 μM, or 1.0 μM, in the perilymph fluid in the inner ear. Alternatively, the tazemetostat is administered to a subject, for example to the middle ear at a concentration of about 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 1 mM, 5 mM, 10 mM, or 50 mM the GSK3-inhibitor XXII is administered, in amount sufficient to achieve a concentration of about 0.1 mM, 0.2 mM, 0.3 mM, 0.4 mM, 0.5 mM, 0.6 mM, 0.7 mM, 0.8 mM, 0.9 mM, or 1.0 mM, in the perilymph fluid in the inner ear.

In some embodiments the EZH2 inhibitor is tazemetostat and the Wnt agonist is CHIR99021. In some embodiments, tazemetostat is administered, in amount sufficient to achieve a concentration of about 0.1 nM, 0.2 nM, 0.3 nM, 0.4 nM, 0.5 nM, 0.6 nM, 0.7 nM, 0.8 nM, 0.9 nM, 1.0 nM, 2.0 nM, 3.0 nM, 4.0 nM, 5.0 nM, 6.0 nM, 7.0 nM, 8.0 nM, 9.0 nM, 10 nM, 20 nM, 30 nM, 40 nM, 50 nM, 60 nM, 70 nM, 80 nM, 90 nM, 100 nM, 200 nM, 300 nM, 400 nM, 500 mM, 1 μM, 5 μM, 10 μM, or 50 μM in the perilymph fluid in the inner ear and CHIR99021 is administered, in amount sufficient to achieve a concentration of about 1 μM, 2 μM, 3 μM, 4 μM, 5 μM, 6 μM, 7 μM, 8 μM, 9 μM, or 10 μM, in the perilymph fluid in the inner ear Alternatively, the tazemetostat is administered to a subject, for example to the middle ear at a concentration of about 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 1 mM, 5 mM, 10 mM, or 50 mM and CHIR99021 is administered to a subject, for example to the middle ear at a concentration of about 1 mM, 2 mM, 3 mM, 4 mM, 5 mM, 6 mM, 7 mM, 8 mM, 9 mM, or 10 mM.

In some embodiments the EZH2 inhibitor is valemetostat and the Wnt agonist is AZD1080. In some embodiments, valemetostat is administered, in amount sufficient to achieve a concentration of about 0.1 nM, 0.2 nM, 0.3 nM, 0.4 nM, 0.5 nM, 0.6 nM, 0.7 nM, 0.8 nM, 0.9 nM, 1.0 nM, 2.0 nM, 3.0 nM, 4.0 nM, 5.0 nM, 6.0 nM, 7.0 nM, 8.0 nM, 9.0 nM, 10 nM, 20 nM, 30 nM, 40 nM, 50 nM, 60 nM, 70 nM, 80 nM, 90 nM, 100 nM, 200 nM, 300 nM, 400 nM, 500 nM, 1 μM, 5 μM, 10 μM, or 50 μM in the perilymph fluid in the inner ear and AZD1080 is administered, in amount sufficient to achieve a concentration of about is about 1 μM, 2 μM, 3 μM, 4 μM, 5 μM, 6 μM, 7 μM, 8 μM, 9 μM, or 10 μM, in the perilymph fluid in the inner ear. Alternatively, the valemetostat is administered to a subject, for example to the middle ear at a concentration of about 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 1 mM, 5 mM, 10 mM, or 50 μM and AZD1080, and is administered to a subject, for example to the middle ear at a concentration of about 1 mM, 2 mM, 3 mM, 4 mM, 5 mM, 6 mM, 7 mM, 8 mM, 9 mM, or 10 mM.

In some embodiments the EZH2 inhibitor is valemetostat and the Wnt agonist is LY2090314 In some embodiments, valemetostat is administered, in amount sufficient to achieve a concentration of about 0.1 nM, 0.2 nM, 0.3 nM, 0.4 nM, 0.5 nM, 0.6 nM, 0.7 nM, 0.8 nM, 0.9 nM, 1.0 nM, 2.0 nM, 3.0 nM, 4.0 nM, 5.0 nM, 6.0 nM, 7.0 nM, 8.0 nM, 9.0 nM, 10 nM, 20 nM, 30 nM, 40 nM, 50 nM, 60 nM, 70 nM, 80 nM, 90 nM, 100 nM, 200 nM, 300 nM, 400 nM, 500 nM, 1 μM, 5 μM, 10 μM, or 50 μM in the perilymph fluid in the inner ear and LY2090314 is administered, in amount sufficient to achieve a concentration of about 1 nM, 5 nM, 10 nM, 15 nM, 20 nM or 40 nM, in the perilymph fluid in the inner ear. Alternatively, the valemetostat is administered to a subject, for example to the middle ear at a concentration of about 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 1 mM, 5 mM, 10 mM, or 50 mM and LY2090314, and is administered to a subject, for example to the middle ear at a concentration of about 1 μM, 5 μM, 10 μM, 15 μM, 20 μM, or 40 μM.

In some embodiments the EZH2 inhibitor is valemetostat and the Wnt agonist is a substituted 3-Imidazo[1,2-a]pyridin-3-yl-4-(1,2,3,4-tetrahydro-[1,4]diazepino-[6,7,1-hi]indol-7-yl)pyrrole-2,5-dione. In some embodiments, valemetostat is administered, in amount sufficient to achieve a concentration of about 0.1 nM, 0.2 nM, 0.3 nM, 0.4 nM, 0.5 nM, 0.6 nM, 0.7 nM, 0.8 nM, 0.9 nM, 1.0 nM, 2.0 nM, 3.0 nM, 4.0 nM, 5.0 nM, 6.0 nM, 7.0 nM, 8.0 nM, 9.0 nM, 10 nM, 20 nM, 30 nM, 40 nM, 50 nM, 60 nM, 70 nM, 80 nM, 90 nM, 100 nM, 200 nM, 300 nM, 400 nM, 500 nM, 1 μM, 5 μM, 10 μM, or 50 μM in the perilymph fluid in the inner ear and the substituted 3-Imidazo[1,2-a]pyridin-3-yl-4-(1,2,3,4-tetrahydro-[1,4]diazepino-[6,7,1-hi]indol-7-yl)pyrrole-2,5-dione is administered, in amount sufficient to achieve a concentration of about 1 nM, 5 nM, 10 nM, 15 nM, 20 nM 50 nM, 100 nM, 250 nM, or 500 nM, in the perilymph fluid in the inner ear. Alternatively, the valemetostat is administered to a subject, for example to the middle ear at a concentration of about 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 1 mM, 5 mM, 10 mM, or 50 mM and the substituted 3-Imidazo[1,2-a]pyridin-3-yl-4-(1,2,3,4-tetrahydro-[1,4]diazepino-[6,7,1-hi]indol-7-yl)pyrrole-2,5-dione and is administered to a subject, for example to the middle ear at a concentration of about 1 μM, 5 μM, 10 μM, 15 μM, 20 μM, 50 μM, 100 μM, 250 μM, or 500 μM.

In some embodiments the EZH2 inhibitor is valemetostat and the Wnt agonist is GSK3 inhibitor XXII. In some embodiments, valemetostat is administered, in amount sufficient to achieve a concentration of about 0.1 nM, 0.2 nM, 0.3 nM, 0.4 nM, 0.5 nM, 0.6 nM, 0.7 nM, 0.8 nM, 0.9 nM, 1.0 nM, 2.0 nM, 3.0 nM, 4.0 nM, 5.0 nM, 6.0 nM, 7.0 nM, 8.0 nM, 9.0 nM, 10 nM, 20 nM, 30 nM, 40 nM, 50 nM, 60 nM, 70 nM, 80 nM, 90 nM, 100 nM, 200 nM, 300 nM, 400 nM, 500 nM, 1 μM, 5 μM, 10 μM, or 50 μM in the perilymph fluid in the inner ear and GSK3-inhibitor XXII is administered, in amount sufficient to achieve a concentration of about 0.1 μM, 0.2 μM, 0.3 μM, 0.4 μM, 0.5 μM, 0.6 μM, 0.7 μM, 0.8 μM, 0.9 μM, or 1.0 μM, in the perilymph fluid in the inner ear. Alternatively, the valemetostat is administered to a subject, for example to the middle ear at a concentration of about 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 1 mM, 5 mM, 10 mM, or 50 mM, the GSK3-inhibitor XXII is administered, in amount sufficient to achieve a concentration of about 0.1 mM, 0.2 mM, 0.3 mM, 0.4 mM, 0.5 mM, 0.6 mM, 0.7 mM, 0.8 mM, 0.9 mM, or 1.0 mM, in the perilymph fluid in the inner ear.

In some embodiments the EZH2 inhibitor is valemetostat and the Wnt agonist is CHIR99021. In some embodiments, valemetostat is administered, in amount sufficient to achieve a concentration of about 0.1 nM, 0.2 nM, 0.3 nM, 0.4 nM, 0.5 nM, 0.6 nM, 0.7 nM, 0.8 nM, 0.9 nM, 1.0 nM, 2.0 nM, 3.0 nM, 4.0 nM, 5.0 nM, 6.0 nM, 7.0 nM, 8.0 nM, 9.0 nM, 10 nM, 20 nM, 30 nM, 40 nM, 50 nM, 60 nM, 70 nM, 80 nM, 90 nM, 100 nM, 200 nM, 300 nM, 400 nM, 500 nM, 1 μM, 5 μM, 10 μM, or 50 μM in the perilymph fluid in the inner ear and CHIR99021 is administered, in amount sufficient to achieve a concentration of about 1 μM, 2 μM, 3 μM, 4 μM, 5 μM, 6 μM, 7 μM, 8 μM, 9 μM, or 10 μM, in the perilymph fluid in the inner ear Alternatively, the valemetostat is administered to a subject, for example to the middle ear at a concentration of about 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 1 mM, 5 mM, 10 mM, or 50 mM and CHIR99021 is administered to a subject, for example to the middle ear at a concentration of about 1 mM, 2 mM, 3 mM, 4 mM, 5 mM, 6 mM, 7 mM, 8 mM, 9 mM, or 10 mM.

In some embodiments the EZH2 inhibitor is E11 and the Wnt agonist is AZD1080. In some embodiments, E11 is administered, in amount sufficient to achieve a concentration of about 0.1 μM, 1 μM, 2 μM, 3 μM, 4 μM, 5 μM, 6 μM, 7 μM, 8 μM, 9 μM, 10 μM, 12 μM, 14 μM, 16 μM, 18 μM or 20 μM in the perilymph fluid in the inner ear and AZD1080 is administered, in amount sufficient to achieve a concentration of about is about 1 μM, 2 μM, 3 μM, 4 μM, 5 μM, 6 μM, 7 μM, 8 μM, 9 μM, or 10 μM, in the perilymph fluid in the inner ear. Alternatively, the E11 to a subject, for example to the middle ear at a concentration of about 0.1 mM, 0.2 mM, 0.3 mM, 0.4 mM, 0.5 mM, 0.6 mM, 0.7 mM, 0.8 mM, 0.9 mM, 1 mM, 2 mM, 3 mM, 4 mM, 5 mM, 6 mM, 7 mM, 8 mM, 9 mM, 10 mM, 12 mM, 14 mM, 16 mM, 18 mM, or 20 mM and AZD1080, and is administered to a subject, for example to the middle ear at a concentration of about 1 mM, 2 mM, 3 mM, 4 mM, 5 mM, 6 mM, 7 mM, 8 mM, 9 mM, or 10 mM.

In some embodiments the EZH2 inhibitor is E11 and the Wnt agonist is LY209031. In some embodiments, E11 is administered, in amount sufficient to achieve a concentration of about 0.1 μM, 1 μM, 2 μM, 3 μM, 4 μM, 5 μM, 6 μM, 7 μM, 8 μM, 9 μM, 10 μM, 12 μM, 14 μM, 16 μM, 18 μM or 20 μM in the perilymph fluid in the inner ear and LY2090314 is administered, in amount sufficient to achieve a concentration of about 1 nM, 5 nM, 10 nM, 15 nM, 20 nM or 40 nM, in the perilymph fluid in the inner ear. Alternatively, the E11 to a subject, for example to the middle ear at a concentration of about 0.1 mM, 0.2 mM, 0.3 mM, 0.4 mM, 0.5 mM, 0.6 mM, 0.7 mM, 0.8 mM, 0.9 mM, 1 mM, 2 mM, 3 mM, 4 mM, 5 mM, 6 mM, 7 mM, 8 mM, 9 mM, 10 mM, 12 mM, 14 mM, 16 mM, 18 mM, or 2.0 mM and LY2090314, and is administered to a subject, for example to the middle ear at a concentration of about 1 μM, 5 μM, 10 μM, 15 μM, 20 μM, or 40 nM.

In some embodiments the EZH2 inhibitor is E11 and the Wnt agonist is a substituted 3-Imidazo[1,2-a]pyridin-3-yl-4-(1,2,3,4-tetrahydro-[1,4]diazepino-[6,7,1-hi]indol-7-yl)pyrrole-2,5-dione. In some embodiments, E11 is administered, in amount sufficient to achieve a concentration of about 0.1 μM, 1 μM, 2 μM, 3 μM, 4 μM, 5 μM, 6 μM, 7 μM, 8 μM, 9 μM, 10 μM, 12 μM, 14 μM, 16 μM, 18 μM, or 20 μM in the perilymph fluid in the inner ear and the substituted 3-Imidazo[1,2-a]pyridin-3-yl-4-(1,2,3,4-tetrahydro-[1,4]diazepino-[6,7,1-hi]indol-7-yl)pyrrole-2,5-dione is administered, in amount sufficient to achieve a concentration of about 1 nM, 5 nM, 10 nM, 15 nM, 20 nM, 50 nM, 100 nM, 250 nM, or 500 nM, in the perilymph fluid in the inner ear. Alternatively, the E11 to a subject, for example to the middle ear at a concentration of about 0.1 mM, 0.2 mM, 0.3 mM, 0.4 mM, 0.5 mM, 0.6 mM, 0.7 mM, 0.8 mM, 0.9 mM, 1 mM, 2 mM, 3 mM, 4 mM, 5 mM, 6 mM, 7 mM, 8 mM, 9 mM, 10 mM, 12 mM, 14 mM, 16 mM, 18 mM or 20 mM and the substituted 3-Imidazo[1,2-a]pyridin-3-yl-4-(1,2,3,4-tetrahydro-[1,4]diazepino-[6,7,1-hi]indol-7-yl)pyrrole-2,5-dione and is administered to a subject, for example to the middle ear at a concentration of about 1 μM, 5 μM, 10 μM, 15 μM, 20 μM, 50 μM, 100 μM, 250 μM, 500 μM.

In some embodiments the EZH2 inhibitor is E11 and the Wnt agonist is GSK3 inhibitor XXII In some embodiments, E11 is administered, in amount sufficient to achieve a concentration of about 0.1 μM, 1 μM, 2 μM, 3 μM, 4 μM, 5 μM, 6 μM, 7 μM, 8 μM, 9 μM, 10 μM, 12 μM, 14 μM, 16 μM, 18 μM, or 20 μM in the perilymph fluid in the inner ear and GSK3-inhibitor XXII is administered, in amount sufficient to achieve a concentration of about 0.1 μM, 0.2 μM, 0.3 μM, 0.4 μM, 0.5 μM, 0.6 μM, 0.7 μM, 0.8 μM, 0.9 μM, or 1.0 μM, in the perilymph fluid in the inner ear. Alternatively, the E11 to a subject, for example to the middle ear at a concentration of about 0.1 mM, 0.2 mM, 0.3 mM, 0.4 mM, 0.5 mM, 0.6 mM, 0.7 mM, 0.8 mM, 0.9 mM, 1 mM, 2 mM, 3 mM, 4 mM, 5 mM, 6 mM, 7 mM, 8 mM, 9 mM, 10 mM, 12 mM, 14 mM, 16 mM, 18 mM, or 20 mM and the GSK3-inhibitor XXII is administered, in amount sufficient to achieve a concentration of about 0.1 mM, 0.2 mM, 0.3 mM, 0.4 mM, 0.5 mM, 0.6 mM, 0.7 mM, 0.8 mM, 0.9 mM, or 1.0 mM, in the perilymph fluid in the inner ear.

In some embodiments the EZH2 inhibitor is E11 and the Wnt agonist is CHIR99021. In some embodiments, E11 is administered, in amount sufficient to achieve a concentration of about 0.1 μM, 1 μM, 2 μM, 3 μM, 4 μM, 5 μM, 6 μM, 7 μM, 8 μM, 9 μM, 10 μM, 12 μM, 14 μM, 16 μM, 18 μM or 20 μM in the perilymph fluid in the inner ear and CHIR99021 is administered, in amount sufficient to achieve a concentration of about 1 μM, 2 μM, 3 μM, 4 μM, 5 μM, 6 μM, 7 μM, 8 μM, 9 μM, or 10 μM, in the perilymph fluid in the inner ear Alternatively, the E11 to a subject, for example to the middle ear at a concentration of about 0.1 mM, 0.2 mM, 0.3 mM, 0.4 mM, 0.5 mM, 0.6 mM, 0.7 mM, 0.8 mM, 0.9 mM, 1 mM, 2 mM, 3 mM, 4 mM, 5 mM, 6 mM, 7 mM, 8 mM, 9 mM, 10 mM, 12 mM, 14 mM, 16 mM, 18 mM, or 20 mM. and CHIR99021 is administered to a subject, for example to the middle ear at a concentration of about 1 mM, 2 mM, 3 mM, 4 mM, 5 mM, 6 mM, 7 mM, 8 mM, 9 mM, or 10 mM.

In some embodiments the EZH2 inhibitor is PF-06821497 and the Wnt agonist is AZD1080. In some embodiments, PF-06821497 is administered, in amount sufficient to achieve a concentration of about 0.1 μM, 0.2 μM, 0.3 μM, 0.4 μM, 0.5 μM, 0.6 μM, 0.7 μM, 0.8 μM, 0.9 μM, 1 μM, 2 μM, 3 μM, 4 μM, 5 μM, 6 μM, 7 μM, 8 μM, 9 μM, or 10 μM in the perilymph fluid in the inner ear and AZD1080 is administered, in amount sufficient to achieve a concentration of about is about 1 μM, 2 μM, 3 μM, 4 μM, 5 μM, 6 μM, 7 μM, 8 μM, 9 μM, or 10 μM, in the perilymph fluid in the inner ear. Alternatively, PF-06821497 is administered to a subject, for example to the middle ear at a concentration of about 0.1 mM, 0.2 mM, 0.3 mM, 0.4 mM, 0.5 mM, 0.6 mM, 0.7 mM, 0.8 mM, 0.9 mM, 1 mM, 2 mM, 3 mM, 4 mM, 5 mM, 6 mM, 7 mM, 8 mM, 9 mM, or 10 mM and AZD1080, and is administered to a subject, for example to the middle ear at a concentration of about 1 mM, 2 mM, 3 mM, 4 mM, 5 mM, 6 mM, 7 mM, 8 mM, 9 mM, or 10 mM.

In some embodiments the EZH2 inhibitor is PF-06821497 and the Wnt agonist is LY209031. In some embodiments, PF-06821497 is administered, in amount sufficient to achieve a concentration of about 0.1 μM, 0.2 μM, 0.3 μM, 0.4 μM, 0.5 μM, 0.6 μM, 0.7 μM, 0.8 μM, 0.9 μM, 1 μM, 2 μM, 3 μM, 4 μM, 5 μM, 6 μM, 7 μM, 8 μM, 9 μM, or 10 μM in the perilymph fluid in the inner ear and LY2090314 is administered, in amount sufficient to achieve a concentration of about 1 nM, 5 nM, 10 nM, 15 nM, 20 nM or 40 nM in the perilymph fluid in the inner ear. Alternatively, PE-06821497 is administered to a subject, for example to the middle ear at a concentration of about 0.1 mM, 0.2 mM, 0.3 mM, 0.4 mM, 0.5 mM, 0.6 mM, 0.7 mM, 0.8 mM, 0.9 mM, 1 mM, 2 mM, 3 mM, 4 mM, 5 mM, 6 mM, 7 mM, 8 mM, 9 mM, or 10 mM and LY2090314, and is administered to a subject, for example to the middle ear at a concentration of about 1 μM, 5 μM, 10 μM, 15 μM, 20 μM, or 40 μM.

In some embodiments the EZH2 inhibitor is PF-06821497 and the Wnt agonist is a substituted 3-Imidazo[1,2-a]pyridin-3-yl-4-(1,2,3,4-tetrahydro-[1,4]diazepino-[6,7,1-hi]indol-7-yl)pyrrole-2,5-dione. In some embodiments, PF-06821497 is administered, in amount sufficient to achieve a concentration of about 0.1 μM, 0.2 μM, 0.3 μM, 0.4 μM, 0.5 μM, 0.6 μM, 0.7 μM, 0.8 μM, 0.9 μM, 1 μM, 2 μM, 3 μM, 4 μM, 5 μM, 6 μM, 7 μM, 8 μM, 9 μM, or 10 μM in the perilymph fluid in the inner ear and the substituted 3-Imidazo[1,2-a]pyridin-3-yl-4-(1,2,3,4-tetrahydro-[1,4]diazepino-[6,7,1-hi]indol-7-yl)pyrrole-2,5-dione is administered, in amount sufficient to achieve a concentration of about 1 nM, 5 nM, 10 nM, 15 nM, 20 nM, 50 nM, 100 nM, 250 nM, or 500 nM, in the perilymph fluid in the inner ear. Alternatively, PF-06821497 is administered to a subject, for example to the middle ear at a concentration of about 0.1 mM, 0.2 mM, 0.3 mM, 0.4 mM, 0.5 mM, 0.6 mM, 0.7 mM, 0.8 mM, 0.9 mM, 1 mM, 2 mM, 3 mM, 4 mM, 5 mM, 6 mM, 7 mM, 8 mM, 9 mM, or 10 mM and the substituted 3-Imidazo[1,2-a]pyridin-3-yl-4-(1,2,3,4-tetrahydro-[1,4]diazepino-[6,7,1-hi]indol-7-yl)pyrrole-2,5-dione and is administered to a subject, for example to the middle ear at a concentration of about 1 μM, 5 μM, 10 μM, 15 μM, 20 μM, 50 μM, 100 μM, 250 μM or 500 μM.

In some embodiments the EZH2 inhibitor is PF-06821497 and the Wnt agonist is GSK3 inhibitor XXII. In some embodiments, PF-06821497 is administered, in amount sufficient to achieve a concentration of about 0.1 μM, 0.2 μM, 0.3 μM, 0.4 μM, 0.5 μM, 0.6 μM, 0.7 μM, 0.8 μM, 0.9 μM, 1 μM, 2 μM, 3 μM, 4 μM, 5 μM, 6 μM, 7 μM, 8 μM, 9 μM, or 10 μM in the perilymph fluid in the inner ear and GSK3-inhibitor XXII is administered, in amount sufficient to achieve a concentration of about 0.1 μM, 0.2 μM, 0.3 μM, 0.4 μM, 0.5 μM, 0.6 μM, 0.7 μM, 0.8 μM, 0.9 μM, or 1.0 μM, in the perilymph fluid in the inner ear Alternatively, PF-06821497 is administered to a subject, for example to the middle ear at a concentration of about 0.1 mM, 0.2 mM, 0.3 mM, 0.4 mM, 0.5 mM, 0.6 mM, 0.7 mM, 0.8 mM, 0.9 mM, 1 mM, 2 mM, 3 mM, 4 mM, 5 mM, 6 mM, 7 mM, 8 mM, 9 mM, or 10 mM and, the GSK3-inhibitor XXII is administered, in amount sufficient to achieve a concentration of about 0.1 mM, 0.2 mM, 0.3 mM, 0.4 mM, 0.5 mM, 0.6 mM, 0.7 mM, 0.8 mM, 0.9 mM, or 1.0 mM, in the perilymph fluid in the inner ear. In some embodiments the EZH2 inhibitor is PF-06821497 and the Wnt agonist is CHIR99021. In some embodiments, PF-06821497 is administered, in amount sufficient to achieve a concentration of about 0.1 μM, 0.2 μM, 0.3 μM, 0.4 μM, 0.5 μM, 0.6 μM, 0.7 μM, 0.8 μM, 0.9 μM, 1 μM, 2 μM, 3 μM, 4 μM, 5 μM, 6 μM, 7 μM, 8 μM, 9 μM, or 10 μM in the perilymph fluid in the inner ear and CHIR99021 is administered, in amount sufficient to achieve a concentration of about 1 μM, 2 μM, 3 μM, 4 μM, 5 μM, 6 μM, 7 μM, 8 μM, 9 μM, or 10 μM, in the perilymph fluid in the inner ear. Alternatively, PF-06821497 is administered to a subject, for example to the middle ear at a concentration of about 0.1 mM, 0.2 mM, 0.3 mM, 0.4 mM, 0.5 mM, 0.6 mM, 0.7 mM, 0.8 mM, 0.9 mM, 1 mM, 2 mM, 3 mM, 4 mM, 5 mM, 6 mM, 7 mM, 8 mM, 9 mM, or 10 mM and CHIR99021 is administered to a subject, for example to the middle ear at a concentration of about 1 mM, 2 mM, 3 mM, 4 mM, 5 mM, 6 mM, 7 mM, 8 mM, 9 mM, or 10 mM.

In some embodiments the EZH2 inhibitor is UNC 2399 and the Wnt agonist is AZD1080. In some embodiments, UNC 2399 is administered, in amount sufficient to achieve a concentration of about 0.1 μM, 0.2 μM, 0.3 μM, 0.4 μM, 0.5 μM, 0.6 μM, 0.7 μM, 0.8 μM, 0.9 μM, 1 μM, 2 μM, 3 μM, 4 μM, 5 μM, 6 μM, 7 μM, 8 μM, 9 μM, or 10 μM in the perilymph fluid in the inner ear and AZD1080 is administered, in amount sufficient to achieve a concentration of about is about 1 μM, 2 μM, 3 μM, 4 μM, 5 μM, 6 μM, 7 μM, 8 μM, 9 μM, or 10 μM, in the perilymph fluid in the inner ear. Alternatively, UNC 2399 is administered to a subject, for example to the middle ear at a concentration of about 0.1 mM, 0.2 mM, 0.3 mM, 0.4 mM, 0.5 mM, 0.6 mM, 0.7 mM, 0.8 mM, 0.9 mM, 1 mM, 2 mM, 3 mM, 4 mM, 5 mM, 6 mM, 7 mM, 8 mM, 9 mM, or 10 mM and AZD1080, and is administered to a subject, for example to the middle ear at a concentration of about 1 mM, 2 mM, 3 mM, 4 mM, 5 mM, 6 mM, 7 mM, 8 mM, 9 mM, or 10 mM.

In some embodiments the EZH2 inhibitor is UNC 2399 and the Wnt agonist is LY209031. In some embodiments, UNC 2399 is administered, in amount sufficient to achieve a concentration of about 0.1 μM, 0.2 μM, 0.3 μM, 0.4 μM, 0.5 μM, 0.6 μM, 0.7 μM, 0.8 μM, 0.9 μM, 1 μM, 2 μM, 3 μM, 4 μM, 5 μM, 6 μM, 7 μM, 8 μM, 9 μM, or 10 μM in the perilymph fluid in the inner ear and LY2090314 is administered, in amount sufficient to achieve a concentration of about 1 nM, 5 nM, 10 nM, 15 nM, 20 nM or 40 nM in the perilymph fluid in the inner ear. Alternatively, UNC 2399 is administered to a subject, for example to the middle ear at a concentration of about 0.1 mM, 0.2 mM, 0.3 mM, 0.4 mM, 0.5 mM, 0.6 mM, 0.7 mM, 0.8 mM, 0.9 mM, 1 mM, 2 mM, 3 mM, 4 mM, 5 mM, 6 mM, 7 mM, 8 mM, 9 mM, or 10 mM and LY2090314, and is administered to a subject, for example to the middle ear at a concentration of about 1 μM, 5 μM, 10 μM, 15 μM, 20 μM, or 40 μM.

In some embodiments the EZH2 inhibitor is UNC 2399 and the Wnt agonist is a substituted 3-Imidazo[1,2-a]pyridin-3-yl-4-(1,2,3,4-tetrahydro-[1,4]diazepino-[6,7,1-hi]indol-7-yl)pyrrole-2,5-dione. In some embodiments, UNC 2399 is administered, in amount sufficient to achieve a concentration of about 0.1 μM, 0.2 μM, 0.3 μM, 0.4 μM, 0.5 μM, 0.6 μM, 0.7 μM, 0.8 μM, 0.9 μM, 1 μM, 2 μM, 3 μM, 4 μM, 5 μM, 6 μM, 7 μM, 8 μM, 9 μM, or 10 μM in the perilymph fluid in the inner ear and the substituted 3-Imidazo[1,2-a]pyridin-3-yl-4-(1,2,3,4-tetrahydro-[1,4]diazepino-[6,7,1-hi]indol-7-yl)pyrrole-2,5-dione is administered, in amount sufficient to achieve a concentration of about 1 nM, 5 nM, 10 nM, 15 nM, 20 nM, 50 nM, 100 nM, 250 nM, or 500 nM, in the perilymph fluid in the inner ear. Alternatively, UNC 2399 is administered to a subject, for example to the middle ear at a concentration of about 0.1 mM, 0.2 mM, 0.3 mM, 0.4 mM, 0.5 mM, 0.6 mM, 0.7 mM, 0.8 mM, 0.9 mM, 1 mM, 2 mM, 3 mM, 4 mM, 5 mM, 6 mM, 7 mM, 8 mM, 9 mM, or 10 mM and the substituted 3-Imidazo[1,2-a]pyridin-3-yl-4-(1,2,3,4-tetrahydro-[1,4]diazepino-[6,7,1-hi]indol-7-yl)pyrrole-2,5-dione and is administered to a subject, for example to the middle ear at a concentration of about 1 μM, 5 μM, 10 μM, 15 μM, 20 μM, 50 μM, 100 μM, 250 μM or 500 μM.

In some embodiments the EZH2 inhibitor is UNC 2399 and the Wnt agonist is GSK3 inhibitor XXII. In some embodiments, UNC 2399 is administered, in amount sufficient to achieve a concentration of about 0.1 μM, 0.2 μM, 0.3 μM, 0.4 μM, 0.5 μM, 0.6 μM, 0.7 μM, 0.8 μM, 0.9 μM, 1 μM, 2 μM, 3 μM, 4 μM, 5 μM, 6 μM, 7 μM, 8 μM, 9 μM, or 10 μM in the perilymph fluid in the inner ear and GSK3-inhibitor XXII is administered, in amount sufficient to achieve a concentration of about 0.1 μM, 0.2 μM, 0.3 μM, 0.4 μM, 0.5 μM, 0.6 μM, 0.7 μM, 0.8 μM, 0.9 μM, or 1.0 μM, in the perilymph fluid in the inner ear. Alternatively, UNC 2399 is administered to a subject, for example to the middle ear at a concentration of about 0.1 mM, 0.2 mM, 0.3 mM, 0.4 mM, 0.5 mM, 0.6 mM, 0.7 mM, 0.8 mM, 0.9 mM, 1 mM, 2 mM, 3 mM, 4 mM, 5 mM, 6 mM, 7 mM, 8 mM, 9 mM, or 10 mM and, the GSK3-inhibitor XXII is administered, in amount sufficient to achieve a concentration of about 0.1 mM, 0.2 mM, 0.3 mM, 0.4 mM, 0.5 mM, 0.6 mM, 0.7 mM, 0.8 mM, 0.9 mM, or 1.0 mM, in the perilymph fluid in the inner ear. In some embodiments the EZH2 inhibitor is UNC 2399 and the Wnt agonist is CHIR99021. In some embodiments, UNC 2399 is administered, in amount sufficient to achieve a concentration of about 0.1 μM, 0.2 μM, 0.3 μM, 0.4 μM, 0.5 μM, 0.6 μM, 0.7 μM, 0.8 μM, 0.9 μM, 1 μM, 2 μM, 3 μM, 4 μM, 5 μM, 6 μM, 7 μM, 8 μM, 9 μM, or 10 μM in the perilymph fluid in the inner ear and CHIR99021 is administered, in amount sufficient to achieve a concentration of about 1 μM, 2 μM, 3 μM, 4 μM, 5 μM, 6 μM, 7 μM, 8 μM, 9 μM, or 10 μM, in the perilymph fluid in the inner ear. Alternatively, UNC 2399 is administered to a subject, for example to the middle ear at a concentration of about 0.1 mM, 0.2 mM, 0.3 mM, 0.4 mM, 0.5 mM, 0.6 mM, 0.7 mM, 0.8 mM, 0.9 mM, 1 mM, 2 mM, 3 mM, 4 mM, 5 mM, 6 mM, 7 mM, 8 mM, 9 mM, or 10 mM and CHIR99021 is administered to a subject, for example to the middle ear at a concentration of about 1 mM, 2 mM, 3 mM, 4 mM, 5 mM, 6 mM, 7 mM, 8 mM, 9 mM, or 10 mM.

In some embodiments the EZH2 inhibitor is CPI-360 and the Wnt agonist is AZD1080. In some embodiments, CPI-360 is administered, in amount sufficient to achieve a concentration of about 0.1 μM, 0.2 μM, 0.3 μM, 0.4 μM, 0.5 μM, 0.6 μM, 0.7 μM, 0.8 μM, 0.9 μM, 1 μM, 2 μM, 3 μM, 4 μM, 5 μM, 6 μM, 7 μM, 8 μM, 9 μM, or 10 μM in the perilymph fluid in the inner ear and AZD1080 is administered, in amount sufficient to achieve a concentration of about is about 1 μM, 2 μM, 3 μM, 4 μM, 5 μM, 6 μM, 7 μM, 8 μM, 9 μM, or 10 μM, in the perilymph fluid in the inner ear. Alternatively, CPI-360 is administered to a subject, for example to the middle ear at a concentration of about 0.1 mM, 0.2 mM, 0.3 mM, 0.4 mM, 0.5 mM, 0.6 mM, 0.7 mM, 0.8 mM, 0.9 mM, 1 mM, 2 mM, 3 mM, 4 mM, 5 mM, 6 mM, 7 mM, 8 mM, 9 mM, or 10 mM and AZD1080, and is administered to a subject, for example to the middle ear at a concentration of about 1 mM, 2 mM, 3 mM, 4 mM, 5 mM, 6 mM, 7 mM, 8 mM, 9 mM, or 10 mM.

In some embodiments the EZH2 inhibitor is CPI-360 and the Wnt agonist is LY209031. In some embodiments, CPI-360 is administered, in amount sufficient to achieve a concentration of about 0.1 μM, 0.2 μM, 0.3 μM, 0.4 μM, 0.5 μM, 0.6 μM, 0.7 μM, 0.8 μM, 0.9 μM, 1 μM, 2 μM, 3 μM, 4 μM, 5 μM, 6 μM, 7 μM, 8 μM, 9 μM, or 10 μM in the perilymph fluid in the inner ear and LY2090314 is administered, in amount sufficient to achieve a concentration of about 1 nM, 5 nM, 10 nM, 15 nM, 20 nM or 40 nM in the perilymph fluid in the inner ear. Alternatively, CPI-360 is administered to a subject, for example to the middle ear at a concentration of about 0.1 mM, 0.2 mM, 0.3 mM, 0.4 mM, 0.5 mM, 0.6 mM, 0.7 mM, 0.8 mM, 0.9 mM, 1 mM, 2 mM, 3 mM, 4 mM, 5 mM, 6 mM, 7 mM, 8 mM, 9 mM, or 10 mM and LY2090314, and is administered to a subject, for example to the middle ear at a concentration of about 1 μM, 5 μM, 10 μM, 15 μM, 20 μM, or 40 μM.

In some embodiments the EZH2 inhibitor is CPI-360 and the Wnt agonist is a substituted 3-Imidazo[1,2-a]pyridin-3-yl-4-(1,2,3,4-tetrahydro-[1,4]diazepino-[6,7,1-hi]indol-7-yl)pyrrole-2,5-dione. In some embodiments, CPI-360 is administered, in amount sufficient to achieve a concentration of about 0.1 μM, 0.2 μM, 0.3 μM, 0.4 μM, 0.5 μM, 0.6 μM, 0.7 μM, 0.8 μM, 0.9 μM, 1 μM, 2 μM, 3 μM, 4 μM, 5 μM, 6 μM, 7 μM, 8 μM, 9 μM, or 10 μM in the perilymph fluid in the inner ear and the substituted 3-Imidazo[1,2-a]pyridin-3-yl-4-(1,2,3,4-tetrahydro-[1,4]diazepino-[6,7,1-hi]indol-7-yl)pyrrole-2,5-dione is administered, in amount sufficient to achieve a concentration of about 1 nM, 5 nM, 10 nM, 15 nM, 20 nM, 50 nM, 100 nM, 250 nM, or 500 nM, in the perilymph fluid in the inner ear. Alternatively, CPI-360 is administered to a subject, for example to the middle ear at a concentration of about 0.1 mM, 0.2 mM, 0.3 mM, 0.4 mM, 0.5 mM, 0.6 mM, 0.7 mM, 0.8 mM, 0.9 mM, 1 mM, 2 mM, 3 mM, 4 mM, 5 mM, 6 mM, 7 mM, 8 mM, 9 mM, or 10 mM and the substituted 3-Imidazo[1,2-a]pyridin-3-yl-4-(1,2,3,4-tetrahydro-[1,4]diazepino-[6,7,1-hi]indol-7-yl)pyrrole-2,5-dione and is administered to a subject, for example to the middle ear at a concentration of about 1 μM, 5 μM, 10 μM, 15 μM, 20 μM, 50 μM, 100 μM, 250 μM or 500 μM.

In some embodiments the EZH2 inhibitor is CPI-360 and the Wnt agonist is GSK3 inhibitor XXII. In some embodiments, CPI-360 is administered, in amount sufficient to achieve a concentration of about 0.1 μM, 0.2 μM, 0.3 μM, 0.4 μM, 0.5 μM, 0.6 μM, 0.7 μM, 0.8 μM, 0.9 μM, 1 μM, 2 μM, 3 μM, 4 μM, 5 μM, 6 μM, 7 μM, 8 μM, 9 μM, or 10 μM in the perilymph fluid in the inner ear and GSK3-inhibitor XXII is administered, in amount sufficient to achieve a concentration of about 0.1 μM, 0.2 μM, 0.3 μM, 0.4 μM, 0.5 μM, 0.6 μM, 0.7 μM, 0.8 μM, 0.9 μM, or 1.0 μM, in the perilymph fluid in the inner ear. Alternatively, CPI-360 is administered to a subject, for example to the middle ear at a concentration of about 0.1 mM, 0.2 mM, 0.3 mM, 0.4 mM, 0.5 mM, 0.6 mM, 0.7 mM, 0.8 mM, 0.9 mM, 1 mM, 2 mM, 3 mM, 4 mM, 5 mM, 6 mM, 7 mM, 8 mM, 9 mM, or 10 mM and, the GSK3-inhibitor XXII is administered, in amount sufficient to achieve a concentration of about 0.1 mM, 0.2 mM, 0.3 mM, 0.4 mM, 0.5 mM, 0.6 mM, 0.7 mM, 0.8 mM, 0.9 mM, or 1.0 mM, in the perilymph fluid in the inner ear. In some embodiments the EZH2 inhibitor is CPI-360 and the Wnt agonist is CHIR99021. In some embodiments, CPI-360 is administered, in amount sufficient to achieve a concentration of about 0.1 μM, 0.2 μM, 0.3 μM, 0.4 μM, 0.5 μM, 0.6 μM, 0.7 μM, 0.8 μM, 0.9 μM, 1 μM, 2 μM, 3 μM, 4 μM, 5 μM, 6 μM, 7 μM, 8 μM, 9 μM, or 10 μM in the perilymph fluid in the inner ear and CHIR99021 is administered, in amount sufficient to achieve a concentration of about 1 μM, 2 μM, 3 μM, 4 μM, 5 μM, 6 μM, 7 μM, 8 μM, 9 μM, or 10 μM, in the perilymph fluid in the inner ear. Alternatively, CPI-360 is administered to a subject, for example to the middle ear at a concentration of about 0.1 mM, 0.2 mM, 0.3 mM, 0.4 mM, 0.5 mM, 0.6 mM, 0.7 mM, 0.8 mM, 0.9 mM, 1 mM, 2 mM, 3 mM, 4 mM, 5 mM, 6 mM, 7 mM, 8 mM, 9 mM, or 10 mM and CHIR99021 is administered to a subject, for example to the middle ear at a concentration of about 1 mM, 2 mM, 3 mM, 4 mM, 5 mM, 6 mM, 7 mM, 8 mM, 9 mM, or 10 mM.

In some embodiments the EZH2 inhibitor is EPZ011989 and the Wnt agonist is AZD1080. In some embodiments, EPZ011989 is administered, in amount sufficient to achieve a concentration of about 0.1 μM, 0.2 μM, 0.3 μM, 0.4 μM, 0.5 μM, 0.6 μM, 0.7 μM, 0.8 μM, 0.9 μM, 1 μM, 2 μM, 3 μM, 4 μM, 5 μM, 6 μM, 7 μM, 8 μM, 9 μM, or 10 μM in the perilymph fluid in the inner ear and AZD1080 is administered, in amount sufficient to achieve a concentration of about is about 1 μM, 2 μM, 3 μM, 4 μM, 5 μM, 6 μM, 7 μM, 8 μM, 9 μM, or 10 μM, in the perilymph fluid in the inner ear. Alternatively, EPZ011989 is administered to a subject, for example to the middle ear at a concentration of about 0.1 mM, 0.2 mM, 0.3 mM, 0.4 mM, 0.5 mM, 0.6 mM, 0.7 mM, 0.8 mM, 0.9 mM, 1 mM, 2 mM, 3 mM, 4 mM, 5 mM, 6 mM, 7 mM, 8 mM, 9 mM, or 10 mM and AZD1080, and is administered to a subject, for example to the middle ear at a concentration of about 1 mM, 2 mM, 3 mM, 4 mM, 5 mM, 6 mM, 7 mM, 8 mM, 9 mM, or 10 mM.

In some embodiments the EZH2 inhibitor is EPZ011989 and the Wnt agonist is LY209031. In some embodiments, EPZ011989 is administered, in amount sufficient to achieve a concentration of about 0.1 μM, 0.2 μM, 0.3 μM, 0.4 μM, 0.5 μM, 0.6 μM, 0.7 μM, 0.8 μM, 0.9 μM, 1 μM, 2 μM, 3 μM, 4 μM, 5 μM, 6 μM, 7 μM, 8 μM, 9 μM, or 10 μM in the perilymph fluid in the inner ear and LY2090314 is administered, in amount sufficient to achieve a concentration of about 1 nM, 5 nM, 10 nM, 15 nM, 20 nM or 40 nM in the perilymph fluid in the inner ear. Alternatively, EPZ011989 is administered to a subject, for example to the middle ear at a concentration of about 0.1 mM, 0.2 mM, 0.3 mM, 0.4 mM, 0.5 mM, 0.6 mM, 0.7 mM, 0.8 mM, 0.9 mM, 1 mM, 2 mM, 3 mM, 4 mM, 5 mM, 6 mM, 7 mM, 8 mM, 9 mM, or 10 mM and LY2090314, and is administered to a subject, for example to the middle ear at a concentration of about 1 μM, 5 μM, 10 μM, 15 μM, 20 μM, or 40 μM.

In some embodiments the EZH2 inhibitor is EPZ011989 and the Wnt agonist is a substituted 3-Imidazo[1,2-a]pyridin-3-yl-4-(1,2,3,4-tetrahydro-[1,4]diazepino-[6,7,1-hi]indol-7-yl)pyrrole-2,5-dione. In some embodiments, EPZ011989 is administered, in amount sufficient to achieve a concentration of about 0.1 μM, 0.2 μM, 0.3 μM, 0.4 μM, 0.5 μM, 0.6 μM, 0.7 μM, 0.8 μM, 0.9 μM, 1 μM, 2 μM, 3 μM, 4 μM, 5 μM, 6 μM, 7 μM, 8 μM, 9 μM, or 10 μM in the perilymph fluid in the inner ear and the substituted 3-Imidazo[1,2-a]pyridin-3-yl-4-(1,2,3,4-tetrahydro-[1,4]diazepino-[6,7,1-hi]indol-7-yl)pyrrole-2,5-dione is administered, in amount sufficient to achieve a concentration of about 1 nM, 5 nM, 10 nM, 15 nM, 20 nM, 50 nM, 100 nM, 250 nM, or 500 nM, in the perilymph fluid in the inner ear. Alternatively, EPZ011989 is administered to a subject, for example to the middle ear at a concentration of about 0.1 mM, 0.2 mM, 0.3 mM, 0.4 mM, 0.5 mM, 0.6 mM, 0.7 mM, 0.8 mM, 0.9 mM, 1 mM, 2 mM, 3 mM, 4 mM, 5 mM, 6 mM, 7 mM, 8 mM, 9 mM, or 10 mM and the substituted 3-Imidazo[1,2-a]pyridin-3-yl-4-(1,2,3,4-tetrahydro-[1,4]diazepino-[6,7,1-hi]indol-7-yl)pyrrole-2,5-dione and is administered to a subject, for example to the middle ear at a concentration of about 1 μM, 5 μM, 10 μM, 15 μM, 20 μM, 50 μM, 100 μM, 250 μM or 500 μM.

In some embodiments the EMU inhibitor is EPZ011989 and the Wnt agonist is GSK3 inhibitor XXII. In some embodiments, EPZ011989 is administered, in amount sufficient to achieve a concentration of about 0.1 μM, 0.2 μM, 0.3 μM, 0.4 μM, 0.5 μM, 0.6 μM, 0.7 μM, 0.8 μM, 0.9 μM, 1 μM, 2 μM, 3 μM, 4 μM, 5 μM, 6 μM, 7 μM, 8 μM, 9 μM, or 10 μM in the perilymph fluid in the inner ear and GSK3-inhibitor XXII is administered, in amount sufficient to achieve a concentration of about 0.1 μM, 0.2 μM, 0.3 μM, 0.4 μM, 0.5 μM, 0.6 μM, 0.7 μM, 0.8 μM, 0.9 μM, or 1.0 μM, in the perilymph fluid in the inner ear. Alternatively, EPZ011989 is administered to a subject, for example to the middle ear at a concentration of about 0.1 mM, 0.2 mM, 0.3 mM, 0.4 mM, 0.5 mM, 0.6 mM, 0.7 mM, 0.8 mM, 0.9 mM, 1 mM, 2 mM, 3 mM, 4 mM, 5 mM, 6 mM, 7 mM, 8 mM, 9 mM, or 10 mM and, the GSK3-inhibitor XXII is administered, in amount sufficient to achieve a concentration of about 0.1 mM, 0.2 mM, 0.3 mM, 0.4 mM, 0.5 mM, 0.6 mM, 0.7 mM, 0.8 mM, 0.9 mM, or 1.0 mM, in the perilymph fluid in the inner ear. In some embodiments the EZH2 inhibitor is EPZ011989 and the Wnt agonist is CHIR99021. In some embodiments, EPZ011989 is administered, in amount sufficient to achieve a concentration of about 0.1 μM, 0.2 μM, 0.3 μM, 0.4 μM, 0.5 μM, 0.6 μM, 0.7 μM, 0.8 μM, 0.9 μM, 1 μM, 2 μM, 3 μM, 4 μM, 5 μM, 6 μM, 7 μM, 8 μM, 9 μM, or 10 μM in the perilymph fluid in the inner ear and CHIR99021 is administered, in amount sufficient to achieve a concentration of about 1 μM, 2 μM, 3 μM, 4 μM, 5 μM, 6 μM, 7 μM, 8 μM, 9 μM, or 10 μM, in the perilymph fluid in the inner ear. Alternatively, EPZ011989 is administered to a subject, for example to the middle ear at a concentration of about 0.1 mM, 0.2 mM, 0.3 mM, 0.4 mM, 0.5 mM, 0.6 mM, 0.7 mM, 0.8 mM, 0.9 mM, 1 mM, 2 mM, 3 mM, 4 mM, 5 mM, 6 mM, 7 mM, 8 mM, 9 mM, or 10 mM and CHIR99021 is administered to a subject, for example to the middle ear at a concentration of about 1 mM, 2 mM, 3 mM, 4 mM, 5 mM, 6 mM, 7 mM, 8 mM, 9 mM, or 10 mM.

In some embodiments the EZH2 inhibitor is PF-06726304 and the Wnt agonist is AZD1080. In some embodiments, PF-06726304 is administered, in amount sufficient to achieve a concentration of about 0.1 nM, 0.2 nM, 0.3 nM, 0.4 nM, 0.5 nM, 0.6 nM, 0.7 nM, 0.8 nM, 0.9 nM, 1.0 nM, 2.0 nM, 3.0 nM, 4.0 nM, 5.0 nM, 6.0 nM, 7.0 nM, 8.0 nM, 9.0 nM, 10 nM, 20 nM, 30 nM, 40 nM, 50 nM, 60 nM, 70 nM, 80 nM, 90 nM, 100 nM, 200 nM, 300 nM, 400 nM, 500 nM, 1 μM, 5 μM, 10 μM, or 50 μM in the perilymph fluid in the inner ear and AZD1080 is administered, in amount sufficient to achieve a concentration of about is about 1 μM, 2 μM, 3 μM, 4 μM, 5 μM, 6 μM, 7 μM, 8 μM, 9 μM, or 10 μM, in the perilymph fluid in the inner ear. Alternatively, the PF-06726304 is administered to a subject, for example to the middle ear at a concentration of about 0.1 μM, 0.2 μM, 0.3 μM, 0.4 μM, 0.5 μM, 0.6 μM, 0.7 μM, 0.8 μM, 0.9 μM,1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 1 mM, 5 mM, 10 mM, or 50 μM and AZD1080, and is administered to a subject, for example to the middle ear at a concentration of about 1 mM, 2 mM, 3 mM, 4 mM, 5 mM, 6 mM, 7 mM, 8 mM, 9 mM, or 10 mM.

In some embodiments the EZH2 inhibitor is PF-06726304 and the Wnt agonist is LY2090314 In some embodiments, PF-06726304 is administered, in amount sufficient to achieve a concentration of about 0.1 nM, 0.2 nM, 0.3 nM, 0.4 nM, 0.5 nM, 0.6 nM, 0.7 nM, 0.8 nM, 0.9 nM, 1.0 nM, 2.0 nM, 3.0 nM, 4.0 nM, 5.0 nM, 6.0 nM, 7.0 nM, 8.0 nM, 9.0 nM, 10 nM, 20 nM, 30 nM, 40 nM, 50 nM, 60 nM, 70 nM, 80 nM, 90 nM, 100 nM, 200 nM, 300 nM, 400 nM, 500 nM, 1 μM, 5 μM, 10 μM, or 50 μM, in the perilymph fluid in the inner ear and LY2090314 is administered, in amount sufficient to achieve a concentration of about 1 nM, 5 nM, 10 nM, 15 nM, 20 nM or 40 nM, in the perilymph fluid in the inner ear. Alternatively, the PF-06726304 is administered to a subject, for example to the middle ear at a concentration of about 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 1 mM, 5 mM, 10 mM, or 50 mM and LY2090314, and is administered to a subject, for example to the middle ear at a concentration of about 1 μM, 5 μM, 10 μM, 15 μM, 20 μM, or 40 μM.

In some embodiments the EZH2 inhibitor is PF-06726304 and the Wnt agonist is a substituted 3-Imidazo[1,2-a]pyridin-3-yl-4-(1,2,3,4-tetrahydro-[1,4]diazepino-[6,7,1-hi]indol-7-yl)pyrrole-2,5-dione. In some embodiments, PF-06726304 is administered, in amount sufficient to achieve a concentration of about 0.1 nM, 0.2 nM, 0.3 nM, 0.4 nM, 0.5 nM, 0.6 nM, 0.7 nM, 0.8 nM, 0.9 nM, 1.0 nM, 2.0 nM, 3.0 nM, 4.0 nM, 5.0 nM, 6.0 nM, 7.0 nM, 8.0 nM, 9.0 nM, 10 nM, 20 nM, 30 nM, 40 nM, 50 nM, 60 nM, 70 nM, 80 nM, 90 nM, 100 nM, 200 nM, 300 nM, 400 nM, 500 nM, 1 μM, 5 μM, 10 μM, or 50 μM in the perilymph fluid in the inner ear and the substituted 3-Imidazo[1,2-a]pyridin-3-yl-4-(1,2,3,4-tetrahydro-[1,4]diazepino-[6,7,1-hi]indol-7-yl)pyrrole-2,5-dione is administered, in amount sufficient to achieve a concentration of about 1 nM, 5 nM, 10 nM, 15 nM, 20 nM, 50 nM, 100 nM, 250 nM, or 500 nM, in the perilymph fluid in the inner ear. Alternatively, the PF-06726304 is administered to a subject, for example to the middle ear at a concentration of about 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 1 mM, 5 mM, 10 mM, or 50 mM and the substituted 3-Imidazo[1,2-a]pyridin-3-yl-4-(1,2,3,4-tetrahydro-[1,4]diazepino-[6,7,1-hi]indol-7-yl)pyrrole-2,5-dione and is administered to a subject, for example to the middle ear at a concentration of about 1 μM, 5 μM, 10 μM, 15 μM, 20 μM, 50 μM, 100 μM, 250 μM, or 500 μM.

In some embodiments the EZH2 inhibitor is PF-06726304 and the Wnt agonist is GSK3 inhibitor XXII. In some embodiments, PF-06726304 is administered, in amount sufficient to achieve a concentration of about 0.1 nM, 0.2 nM, 0.3 nM, 0.4 nM, 0.5 nM, 0.6 nM, 0.7 nM, 0.8 nM, 0.9 nM, 1.0 nM, 2.0 nM, 3.0 nM, 4.0 nM, 5.0 nM, 6.0 nM, 7.0 nM, 8.0 nM, 9.0 nM, 10 nM, 20 nM, 30 nM, 40 nM, 50 nM, 60 nM, 70 nM, 80 nM, 90 nM, 100 nM, 200 nM, 300 nM, 400 nM, 500 nM, 1 μM, 5 μM, 10 μM, or 50 μM in the perilymph fluid in the inner ear and GSK3-inhibitor XXII is administered, in amount sufficient to achieve a concentration of about 0.1 μM, 0.2 μM, 0.3 μM, 0.4 μM, 0.5 μM, 0.6 μM, 0.7 μM, 0.8 μM, 0.9 μM, or 1.0 μM, in the perilymph fluid in the inner ear. Alternatively, the PF-06726304 is administered to a subject, for example to the middle ear at a concentration of about 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 1 mM, 5 mM, 10 mM, or 50 mM, the GSK3-inhibitor XXII is administered, in amount sufficient to achieve a concentration of about 0.1 mM, 0.2 mM, 0.3 mM, 0.4 mM, 0.5 mM, 0.6 mM, 0.7 mM, 0.8 mM, 0.9 mM, or 1.0 mM, in the perilymph fluid in the inner ear.

In some embodiments the EZH2 inhibitor is PF-06726304 and the Wnt agonist is CHIR99021. In some embodiments, PF-06726304 is administered, in amount sufficient to achieve a concentration of about 0.1 nM, 0.2 nM, 0.3 nM, 0.4 nM, 0.5 nM, 0.6 nM, 0.7 nM, 0.8 nM, 0.9 nM, 1.0 nM, 2.0 nM, 3.0 nM, 4.0 nM, 5.0 nM, 6.0 nM, 7.0 nM, 8.0 nM, 9.0 nM, 10 nM, 20 nM, 30 nM, 40 nM, 50 nM, 60 nM, 70 nM, 80 nM, 90 nM, 100 nM, 200 nM, 300 nM, 400 nM, 500 nM, 1 μM, 5 μM, 10 μM, or 50 μM in the perilymph fluid in the inner ear and CHIR99021 is administered, in amount sufficient to achieve a concentration of about 1 μM, 2 μM, 3 μM, 4 μM, 5 μM, 6 μM, 7 μM, 8 μM, 9 μM, or 10 μM, in the perilymph fluid in the inner ear Alternatively, the PF-06726304 is administered to a subject, for example to the middle ear at a concentration of about 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 1 mM, 5 mM, 10 mM, or 50 mM and CHIR99021 is administered to a subject, for example to the middle ear at a concentration of about 1 mM, 2 mM, 3 mM, 4 mM, 5 mM, 6 mM, 7 mM, 8 mM, 9 mM, or 10 mM.

In some embodiments the EZH2 inhibitor is CPI-1205, the Wnt agonist is AZD1080 and the second epigenetic agent is VPA. In some embodiments, CPI-1205 is administered, in amount sufficient to achieve a concentration of about 0.1 nM, 0.2 nM, 0.3 nM, 0.4 nM, 0.5 nM, 0.6 nM, 0.7 nM, 0.8 nM, 0.9 nM, 1.0 nM, 2.0 nM, 3.0 nM, 4.0 nM, 5.0 nM, 6.0 nM, 7.0 nM, 8.0 nM, 9.0 nM, 10 nM, 20 nM, 30 nM, 40 nM, 50 nM, 60 nM, 70 nM, 80 nM, 90 nM, 100 nM, 200 nM, 300 nM, 400 nM, 500 nM, 600 nM, 700 nM, 800 nM, 900 nM, 1 μM, 2 μM, 3 μM, 4 μM, 5 μM, 6 μM, 7 μM, 8 μM, 9 μM, 10 μM, 12 μM, 14 μM, 16 μM, 18 μM, 20 μM, 25 μM or about 30 μM in the perilymph fluid in the inner ear; AZD1080 is administered, in amount sufficient to achieve a concentration of about is about 1 μM, 2 μM, 3 μM, 4 μM, 5 μM, 6 μM, 7 μM, 8 μM, 9 μM, or 10 μM in the perilymph fluid in the inner ear and VPA is administered in amount sufficient to achieve a concentration of about is about 100 μM to 4 mM in the perilymph fluid in the inner ear. Alternatively, the CPI-1205 is administered to a subject, for example to the middle ear at a concentration of about 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, 10 mM, 12 mM, 14 mM, 16 mM, 18 mM, 20 mM, 25 mM, or about 30 mM; AZD1080 is administered to a subject, for example to the middle ear at a concentration of about 1 mM, 2 mM, 3 mM, 4 mM, 5 mM, 6 mM, 7 mM, 8 mM, 9 mM, or 10 mM and VPA to a subject, for example to the middle ear at a concentration about 100 mM to 4,000 mM.

In some embodiments the EZH2 inhibitor is CPI-1205, the Wnt agonist is LY2090314 and the second epigenetic agent is VPA. In some embodiments, CPI-1205 is administered, in amount sufficient to achieve a concentration of about 0.1 nM, 0.2 nM, 0.3 nM, 0.4 nM, 0.5 nM, 0.6 nM, 0.7 nM, 0.8 nM, 0.9 nM, 1.0 nM, 2.0 nM, 3.0 nM, 4.0 nM, 5.0 nM, 6.0 nM, 7.0 nM, 8.0 nM, 9.0 nM, 10 nM, 20 nM, 30 nM, 40 nM, 50 nM, 60 nM, 70 nM, 80 nM, 90 nM, 100 nM, 200 nM, 300 nM, 400 nM, 500 nM, 600 nM, 700 nM, 800 nM, 900 nM, 1 μM, 2 μM, 3 μM, 4 μM, 5 μM, 6 μM, 7 μM, 8 μM, 9 μM, 10 μM, 12 μM, 14 μM, 16 μM, 18 μM, 20 μM, 25 μM, or about 30 μM in the perilymph fluid in the inner ear; LY 2090314 is administered, in amount sufficient to achieve a concentration of about 1 nM, 5 nM, 10 nM, 15 nM, 20 nM or 40 nM in the perilymph fluid in the inner ear and VPA is administered in amount sufficient to achieve a concentration of about is about 100 μM to 4 mM in the perilymph fluid in the inner ear. Alternatively, the CPI-1205 is administered to a subject, for example to the middle ear at a concentration of about 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, 10 mM, 12 mM, 14 mM, 16 mM, 18 mM, 20 mM, 25 mM, or about 30 mM and LY2090314, and is administered to a subject, for example to the middle ear at a concentration of about 1 μM, 5 μM, 10 μM, 15 μM, 20 μM, or 40 μM and VPA is administers to a subject, for example to the middle ear at a concentration about 100 mM to 4,000 mM.

In some embodiments the EZH2 inhibitor is CPI-1205 and the Wnt agonist is a substituted 3-imidazo[1,2-a]pyridin-3-yl-4-(1,2,3,4-tetra hydro-[1,4]diazepino-[6,7,1-hi]indol-7-yl)pyrrole-2,5-dione and the second epigenetic agent is VPA. In some embodiments, CPI-1205 is administered, in amount sufficient to achieve a concentration of about 0.1 nM, 0.2 nM, 0.3 nM, 0.4 nM, 0.5 nM, 0.6 nM, 0.7 nM, 0.8 nM, 0.9 nM, 1.0 nM, 2.0 nM, 3.0 nM, 4.0 nM, 5.0 nM, 6.0 nM, 7.0 nM, 8.0 nM, 9.0 nM, 10 nM, 20 nM, 30 nM, 40 nM, 50 nM, 60 nM, 70 nM, 80 nM, 90 nM, 100 nM, 200 nM, 300 nM, 400 nM, 500 nM, 600 nM, 700 nM, 800 nM, 900 nM, 1 μM, 2 μM, 3 μM, 4 μM, 5 μM, 6 μM, 7 μM, 8 μM, 9 μM, 10 μM, 12 μM, 14 μM, 16 μM, 18 μM, 20 μM, 25 μM, or about 30 μM in the perilymph fluid in the inner ear; the substituted 3-Imidazo[1,2-a]pyridin-3-yl-4-(1,2,3,4-tetrahydro-[1,4]diazepino-[6,7,1-hi]indol-7-yl)pyrrole-2,5-dione is administered, in amount sufficient to achieve a concentration of about 1 nM, 5 nM, 10 nM, 15 nM, 20 nM, 50 nM, 100 nM, 250 nM, or 500 nM, in the perilymph fluid in the inner ear and VPA is administered in amount sufficient to achieve a concentration of about is about 100 μM to 4 mM in the perilymph fluid in the inner ear. Alternatively, the CPI-1205 is administered to a subject, for example to the middle ear at a concentration of about 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, 10 mM, 12 mM, 14 mM, 16 mM, 18 mM, 20 mM, 25 mM, or about 30 mM and the substituted 3-Imidazo[1,2-a]pyridin-3-yl-4-(1,2,3,4-tetrahydro-[1,4]diazepino-[6,7,1-hi]indol-7-yl)pyrrole-2,5-dione and is administered to a subject, for example to the middle ear at a concentration of about 1 μM, 5 μM, 10 μM, 15 μM, 20 μM, 50 μM, 100 μM, 250 μM, or 500 μM and VPA is administers to a subject, for example to the middle ear at a concentration about 100 mM to 4,000 mM.

In some embodiments the EZH2 inhibitor is CPI-1205 and the Wnt agonist is GSK3 inhibitor XXII and the second epigenetic agent is VPA. In some embodiments, CPI-1205 is administered, in amount sufficient to achieve a concentration of about 0.1 nM, 0.2 nM, 0.3 nM, 0.4 nM, 0.5 nM, 0.6 nM, 0.7 nM, 0.8 nM, 0.9 nM, 1.0 nM, 2.0 nM, 3.0 nM, 4.0 nM, 5.0 nM, 6.0 nM, 7.0 nM, 8.0 nM, 9.0 nM, 10 nM, 20 nM, 30 nM, 40 nM, 50 nM, 60 nM, 70 nM, 80 nM, 90 nM, 100 nM, 200 nM, 300 nM, 400 nM, 500 nM, 600 nM, 700 nM, 800 nM, 900 nM, 1 μM, 2 μM, 3 μM, 4 μM, 5 μM, 6 μM, 7 μM, 8 μM, 9 μM, 10 μM, 12 μM, 14 μM, 16 μM, 18 μM, 20 μM, 25 μM, or about 30 μM in the perilymph fluid in the inner ear; the GSK3-inhibitor XXII is administered, in amount sufficient to achieve a concentration of about 0.1 μM, 0.2 μM, 0.3 μM, 0.4 μM, 0.5 μM, 0.6 μM, 0.7 μM, 0.8 μM, 0.9 μM, or 1.0 μM, in the perilymph fluid in the inner ear and VPA is administered in amount sufficient to achieve a concentration of about is about 10 100 μM to 4 mM in the perilymph fluid in the inner ear. Alternatively, the CPI-1205 is administered to a subject, for example to the middle ear at a concentration of about 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, 10 mM, 12 mM, 14 mM, 16 mM, 18 mM, 20 mM, 25 mM, or about 30 mM and the GSK3-inhibitor XXII is administered, in amount sufficient to achieve a concentration of about 0.1 mM, 0.2 mM, 0.3 mM, 0.4 mM, 0.5 mM, 0.6 mM, 0.7 mM, 0.8 mM, 0.9 mM, or 1.0 mM, in the perilymph fluid in the inner ear and VPA is administers to a subject, for example to the middle ear at a concentration about 100 mM to 4,000 mM.

In some embodiments the EZH2 inhibitor is CPI-1205 and the Wnt agonist is CHIR99021 and the second epigenetic agent is VPA. In some embodiments, CPI-1205 is administered, in amount sufficient to achieve a concentration of about 0.1 nM, 0.2 nM, 0.3 nM, 0.4 nM, 0.5 nM, 0.6 nM, 0.7 nM, 0.8 nM, 0.9 nM, 1.0 nM, 2.0 nM, 3.0 nM, 4.0 nM, 5.0 nM, 6.0 nM, 7.0 nM, 8.0 nM, 9.0 nM, 10 nM, 20 nM, 30 nM, 40 nM, 50 nM, 60 nM, 70 nM, 80 nM, 90 nM, 100 nM, 200 nM, 300 nM, 400 nM, 500 nM, 600 nM, 700 nM, 800 nM, 900 nM, 1 μM, 2 μM, 3 μM, 4 μM, 5 μM, 6 μM, 7 μM, 8 μM, 9 μM, 10 μM, 12 μM, 14 μM, 16 μM, 18 μM, 20 μM, 25 μM, or about 30 μM in the perilymph fluid in the inner ear; CRIR99021 is administered, in amount sufficient to achieve a concentration of about 1 μM, 2 μM, 3 μM, 4 μM, 5 μM, 6 μM, 7 μM, 8 μM, 9 μM, or 10 μM, in the perilymph fluid in the inner ear and VPA is administered in amount sufficient to achieve a concentration of about is about 100 μM to 4 mM in the perilymph fluid in the inner ear. Alternatively, the CPI-1205 is administered to a subject, for example to the middle ear at a concentration of about 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, 10 mM, 12 mM, 14 mM, 16 mM, 18 mM, 20 mM, 25 mM, or about 30 mM and CHIR99021 is administered to a subject, for example to the middle ear at a concentration of about 1 mM, 2 mM, 3 mM, 4 mM, 5 mM, 6 mM, 7 mM, 8 mM, 9 mM, or 10 mM and VPA is administers to a subject, for example to the middle ear at a concentration about 100 mM to 4,000 mM.

In some embodiments the EZH2 inhibitor is CPI-169 and the Wnt agonist is AZD1080 and the second epigenetic agent is VPA. In some embodiments, CPI-169 is administered, in amount sufficient to achieve a concentration of about 0.1 nM, 0.2 nM, 0.3 nM, 0.4 nM, 0.5 nM, 0.6 nM, 0.7 nM, 0.8 nM, 0.9 nM, 1.0 nM, 2.0 nM, 3.0 nM, 4.0 nM, 5.0 nM, 6.0 nM, 7.0 nM, 8.0 nM, 9.0 nM, 10 nM, 20 nM, 30 nM, 40 nM, 50 nM, 60 nM, 70 nM, 80 nM, 90 nM, 100 nM, 200 nM, 300 nM, 400 nM, 500 nM, 1 μM, 5 μM, 10 μM, or 50 μM in the perilymph fluid in the inner ear; AZD1.080 is administered, in amount sufficient to achieve a concentration of about is about 1 μM, 2 μM, 3 μM, 4 μM, 5 μM, 6 μM, 7 μM, 8 μM, 9 μM, or 10 μM, in the perilymph fluid in the inner ear and VPA is administered in amount sufficient to achieve a concentration of about is about 100 μM to 4 mM in the perilymph fluid in the inner ear. Alternatively, the CPI-169 is administered to a subject, for example to the middle ear at a concentration of about 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 1 mM, 5 mM, 10 mM, or 50 mM and AZD1080, and is administered to a subject, for example to the middle ear at a concentration of about 1 mM, 2 mM, 3 mM, 4 mM, 5 mM, 6 mM, 7 mM, 8 mM, 9 mM, or 10 mM and VPA is administers to a subject, for example to the middle ear at a concentration about 100 mM to 4,000 mM.

In some embodiments the EZH2 inhibitor is CPI-169 and the Wnt agonist is LY2090314 and the second epigenetic agent is VPA. In some embodiments, CPI-169 is administered, in amount sufficient to achieve a concentration of about 0.1 nM, 0.2 nM, 0.3 nM, 0.4 nM, 0.5 nM, 0.6 nM, 0.7 nM, 0.8 nM, 0.9 nM, 1.0 nM, 2.0 nM, 3.0 nM, 4.0 nM, 5.0 nM, 6.0 nM, 7.0 nM, 8.0 nM, 9.0 nM, 10 nM, 20 nM, 30 nM, 40 nM, 50 nM, 60 nM, 70 nM, 80 nM, 90 nM, 100 nM, 200 nM, 300 nM, 400 nM, 500 nM, 1 μM, 5 μM, 10 μM, or 50 μM in the perilymph fluid in the inner ear; LY2090314 is administered, in amount sufficient to achieve a concentration of about 1 nM, 5 nM, 10 nM, 15 nM, 20 nM or 40 nM in the perilymph fluid in the inner ear and VPA is administered in amount sufficient to achieve a concentration of about is about 100 μM to 4 mM in the perilymph fluid in the inner ear. Alternatively, the CPI-169 is administered to a subject, for example to the middle ear at a concentration of about 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 1 mM, 5 mM, 10 mM, or 50 mM and LY2090314, and is administered to a subject, for example to the middle ear at a concentration of about 1 μM, 5 μM, 10 μM, 15 μM, 20 μM or 40 μM and VPA is administers to a subject, for example to the middle ear at a concentration about 100 mM to 4,000 mM.

In some embodiments the EZH2 inhibitor is CPI-169 and the Wnt agonist is a substituted 3-Imidazo[1,2-a]pyridin-3-yl-4-(1,2,3,4-tetrahydro-[1,4]diazepino-[6,7,1-hi]indol-7-yl)pyrrole-2,5-dione and the second epigenetic agent is VPA. In some embodiments, CPI-169 is administered, in amount sufficient to achieve a concentration of about 0.1 nM, 0.2 nM, 0.3 nM, 0.4 nM, 0.5 nM, 0.6 nM, 0.7 nM, 0.8 nM, 0.9 nM, 1.0 nM, 2.0 nM, 3.0 nM, 4.0 nM, 5.0 nM, 6.0 nM, 7.0 nM, 8.0 nM, 9.0 nM, 10 nM, 20 nM, 30 nM, 40 nM, 50 nM, 60 nM, 70 nM, 80 nM, 90 nM, 100 nM, 200 nM, 300 nM, 400 nM, 500 nM, 1 μM, 5 μM, 10 μM, or 50 μM in the perilymph fluid in the inner ear; the substituted 3-Imidazo[1,2-a]pyridin-3-yl-4-(1,2,3,4-tetrahydro-[1,4]diazepino-[6,7,1-hi]indol-7-yl)pyrrole-2,5-dione is administered, in amount sufficient to achieve a concentration of about 1 nM, 5 nM, 10 nM, 15 nM, 20 nM, 50 nM, 100 nM, 250 nM, or 500 nM, in the perilymph fluid in the inner ear and VPA is administered in amount sufficient to achieve a concentration of about is about 100 μM to 4 mM in the perilymph fluid in the inner ear. Alternatively, the CPI-169 is administered to a subject, for example to the middle ear at a concentration of about 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 1 mM, 5 mM, 10 mM, or 50 mM and the substituted 3-Imidazo[1,2-a]pyridin-3-yl-4-(1,2,3,4-tetrahydro-[1,4]diazepino-[6,7,1-hi]indol-7-yl)pyrrole-2,5-dione and is administered to a subject, for example to the middle ear at a concentration of about 1 μM, 5 μM, 10 μM, 15 μM, 20 μM, 50 μM, 100 μM, 250 μM, or 500 μM and VPA is administers to a subject, for example to the middle ear at a concentration about 100 mM to 4,000 mM.

In some embodiments the EZH2 inhibitor is CPI-169 and the Wnt agonist is GSK3 inhibitor XXII and the second epigenetic agent is VPA. In some embodiments, CPI-169 is administered, in amount sufficient to achieve a concentration of about 0.1 nM, 0.2 nM, 0.3 nM, 0.4 nM, 0.5 nM, 0.6 nM, 0.7 nM, 0.8 nM, 0.9 nM, 1.0 nM, 2.0 nM, 3.0 nM, 4.0 nM, 5.0 nM, 6.0 nM, 7.0 nM, 8.0 nM, 9.0 nM, 10 nM, 20 nM, 30 nM, 40 nM, 50 nM, 60 nM, 70 nM, 80 nM, 90 nM, 100 nM, 200 nM, 300 nM, 400 nM, 500 nM, 1 μM, 5 μM, 10 μM, or 50 μM in the perilymph fluid in the inner ear; GSK3-inhibitor XXII is administered, in amount sufficient to achieve a concentration of about 0.1 μM, 0.2 μM, 0.3 μM, 0.4 μM, 0.5 μM, 0.6 μM, 0.7 μM, 0.8 μM, 0.9 μM, or 1.0 μM, in the perilymph fluid in the inner ear and VPA is administered in amount sufficient to achieve a concentration of about is about 100 μM to 4 mM in the perilymph fluid in the inner ear. Alternatively, the CPI-169 is administered to a subject, for example to the middle ear at a concentration of about 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 nM, 50 μM, 60 μM, 70 μM, 80 μM, 90 μM, 100 μM, 200 μM, 300 μM, 400 μM, 500 μM, 1 mM, 5 mM, 10 mM, or 50 mM and the GSK3-inhibitor XXII is administered to a subject, for example to the middle ear at a concentration about 0.1 mM, 0.2 mM, 0.3 mM, 0.4 mM, 0.5 mM, 0.6 mM, 0.7 mM, 0.8 mM, 0.9 mM, or 1.0 mM and VPA is administered to a subject, for example to the middle ear at a concentration about 100 mM to 4,000 mM.

In some embodiments the EZH2 inhibitor is CPI-169 and the Wnt agonist is CHIR99021 and the second epigenetic agent is VPA. In some embodiments, CPI-169 is administered, in amount sufficient to achieve a concentration of about 0.1 nM, 0.2 nM, 0.3 nM, 0.4 mM, 0.5 nM, 0.6 nM, 0.7 nM, 0.8 nM, 0.9 nM, 1.0 nM, 2.0 nM, 3.0 nM, 4.0 nM, 5.0 nM, 6.0 nM, 7.0 nM, 8.0 nM, 9.0 nM, 10 nM, 20 nM, 30 nM, 40 nM, 50 nM, 60 nM, 70 nM, 80 nM, 90 nM, 100 nM, 200 nM, 300 nM, 400 nM, 500 nM, 1 μM, 5 μM, 10 μM, or 50 μM in the perilymph fluid in the inner ear; CHIR99021 is administered, in amount sufficient to achieve a concentration of about 1 μM, 2 μM, 3 μM, 4 μM, 5 μM, 6 μM, 7 μM, 8 μM, 9 μM, or 10 μM, in the perilymph fluid in the inner ear and VPA is administered in amount sufficient to achieve a concentration of about is about 100 μM to 4 mM in the perilymph fluid in the inner ear. Alternatively, the CPI-169 is administered to a subject, for example to the middle ear at a concentration of about 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 1 mM, 5 mM, 10 mM, or 50 mM and CHIR99021 is administered to a subject, for example to the middle ear at a concentration of about 1 mM, 2 mM, 3 mM, 4 mM, 5 mM, 6 mM, 7 mM, 8 mM, 9 mM, or 10 mM and VPA is administered to a subject, for example to the middle ear at a concentration about 100 mM to 4,000 mM.

In some embodiments the EZH2 inhibitor is E11 and the Wnt agonist is AZD1080 and the second epigenetic agent is VPA. In some embodiments, E11 is administered, in amount sufficient to achieve a concentration of about 0.1 μM, 1 μM, 2 μM, 3 μM, 4 μM, 5 μM, 6 μM, 7 μM, 8 μM, 9 μM, 10 μM, 12 μM, 14 μM, 16 μM, 18 μM or 20 μM in the perilymph fluid in the inner ear and AZD1080 is administered, in amount sufficient to achieve a concentration of about is about 1 μM, 2 μM, 3 μM, 4 μM, 5 μM, 6 μM, 7 μM, 8 μM, 9 μM, or 10 μM in the perilymph fluid in the inner ear and VPA is administered in amount sufficient to achieve a concentration of about is about 100 μM to 4 mM in the perilymph fluid in the inner ear. Alternatively, the E11 to a subject, for example to the middle ear at a concentration of about 0.1 mM, 0.2 mM, 0.3 mM, 0.4 mM, 0.5 mM, 0.6 mM, 0.7 mM, 0.8 mM, 0.9 mM, 1 mM, 2 mM, 3 mM, 4 mM, 5 mM, 6 mM, 7 mM, 8 mM, 9 mM, 10 mM, or 10 mM and AZD1080, and is administered to a subject, for example to the middle ear at a concentration of about 1 mM, 2 mM, 3 mM, 4 mM, 5 mM, 6 mM, 7 mM, 8 mM, 9 mM, or 10 mM and VPA is administered to a subject, for example to the middle ear at a concentration about 100 mM to 4,000 mM.

In some embodiments the EZH2 inhibitor is E11 and the Wnt agonist is LY209031 and the second epigenetic agent is VPA. In some embodiments, E11 is administered, in amount sufficient to achieve a concentration of about 0.11 μM, 1 μM, 2 μM, 3 μM, 4 μM, 5 μM, 6 μM, 7 μM, 8 μM, 9 μM, 10 μM, 12 μM, 14 μM, 16 μM, 18 μM or 20 μM in the perilymph fluid in the inner ear and LY2090314 is administered, in amount sufficient to achieve a concentration of about 1 nM, 5 nM, 10 nM, 15 nM, 20 nM or 40 nM in the perilymph fluid in the inner ear and VPA is administered in amount sufficient to achieve a concentration of about is about 100 μM to 4 mM in the perilymph fluid in the inner ear. Alternatively, the E11 to a subject, for example to the middle ear at a concentration of about 0.1 mM, 0.2 mM, 0.3 mM, 0.4 mM, 0.5 mM, 0.6 mM, 0.7 mM, 0.8 mM, 0.9 mM, 1 mM, 2 mM, 3 mM, 4 mM, 5 mM, 6 mM, 7 mM, 8 mM, 9 mM, 10 mM, 12 mM, 14 mM, 16 mM, 18 mM, or 20 mM and LY2090314, and is administered to a subject, for example to the middle ear at a concentration of about 1 μM, 5 μM, 10 μM, 15 μM, or 20 μM and VPA is administered to a subject, for example to the middle ear at a concentration about 100 mM to 4,000 mM.

In some embodiments the EZH2 inhibitor is E11 and the Wnt agonist is a substituted 3-Imidazo[1,2-a]pyridin-3-yl-4-(1,2,3,4-tetrahydro-[1,4]diazepino-[6,7,1-hi]indol-7-yl)pyrrole-2,5-dime and the second epigenetic agent is VPA. In some embodiments, E11 is administered, in amount sufficient to achieve a concentration of about 0.1 μM, 1 μM, 2 μM, 3 μM, 4 μM, 5 μM, 6 μM, 7 μM, 8 μM, 9 μM, 10 μM, 12 μM, 14 μM, 16 μM, 18 μM or 20 μM in the perilymph fluid in the inner ear and the substituted 3-Imidazo[1,2-a]pyridin-3-yl-4-(1,2,3,4-tetrahydro-[1,4]diazepino-[6,7,1-hi]indol-7-yl)pyrrole-2,5-dione is administered, in amount sufficient to achieve a concentration of about 1 nM, 5 nM, 10 nM, 15 nM, 20 nM, 50 nM, 100 nM, 250 nM, or 500 nM, in the perilymph fluid in the inner ear and VPA is administered in amount sufficient to achieve a concentration of about is about 100 μM to 4 mM in the perilymph fluid in the inner ear. Alternatively, the E11 to a subject, for example to the middle ear at a concentration of about 0.1 mM, 0.2 mM, 0.3 mM, 0.4 mM, 0.5 mM, 0.6 mM, 0.7 mM, 0.8 mM, 0.9 mM, 1 mM, 2 mM, 3 mM, 4 mM, 5 mM, 6 mM, 7 mM, 8 mM, 9 mM, 10 mM, 12 mM, 14 mM, 16 mM, 18 mM, or 20 mM and the substituted 3-Imidazo[1,2-a]pyridin-3-yl-4-(1,2,3,4-tetrahydro-[1,4]diazepino-[6,7,1-hi]indol-7-yl)pyrrole-2,5-dione and is administered to a subject, for example to the middle ear at a concentration of about 1 μM, 5 μM, 10 μM, 15 μM, 20 μM, 50 μM, 100 μM, 250 μM, or 500 μM and NTA is administered to a subject, for example to the middle ear at a concentration about 100 mM to 4,000 mM.

In some embodiments the EZH2 inhibitor is E11 and the Wnt agonist is GSK3 inhibitor XXII and the second epigenetic agent is VPA. In some embodiments, E11 is administered, in amount sufficient to achieve a concentration of about 0.1 μM, 1 μM, 2 μM, 3 μM, 4 μM, 5 μM, 6 μM, 7 μM, 8 μM, 9 μM, 10 μM 12 μM, 14 μM, 16 μM, 18 μM or 20 μM in the perilymph fluid in the inner ear and GSK3-inhibitor XXII is administered, in amount sufficient to achieve a concentration of about 0.1 μM, 0.2 μM, 0.3 μM, 0.4 μM, 0.5 μM, 0.6 μM, 0.7 μM, 0.8 μM, 0.9 μM, or 1.0 μM, in the perilymph fluid in the inner ear and VPA is administered in amount sufficient to achieve a concentration of about is about 100 μM to 4 mM in the perilymph fluid in the inner ear. Alternatively, the E11 to a subject, for example to the middle ear at a concentration of about 0.1 mM, 0.2 mM, 0.3 mM, 0.4 mM, 0.5 mM, 0.6 mM, 0.7 mM, 0.8 mM, 0.9 mM, 1 mM, 2 mM, 3 mM, 4 mM, 5 mM, 6 mM, 7 mM, 8 mM, 9 mM, 10 mM, 12 mM, 14 mM, 16 mM, 18 mM, or 20 mM and the GSK3-inhibitor XXII is administered, in amount sufficient to achieve a concentration of about 0.1 mM, 0.2 mM, 0.3 mM, 0.4 mM, 0.5 mM, 0.6 mM, 0.7 mM, 0.8 mM, 0.9 mM, or 1.0 mM in the perilymph fluid in the inner ear and VPA is administered to a subject, for example to the middle ear at a concentration about 100 mM to 4,000 mM.

In some embodiments the EZH2 inhibitor is E11 and the Wnt agonist is CHIR99021 and the second epigenetic agent is VPA. In some embodiments, E11 is administered, in amount sufficient to achieve a concentration of about 0.1 μM, 1 μM, 2 μM, 3 μM, 4 μM, 5 μM, 6 μM, 7 μM, 8 μM, 9 μM, 10 μM, 12 μM, 14 μM, 16 μM, 18 μM or 20 μM in the perilymph fluid in the inner ear and CHIR99021 is administered, in amount sufficient to achieve a concentration of about 1 μM, 2 μM, 3 μM, 4 μM, 5 μM, 6 μM, 7 μM, 8 μM, 9 μM, or 10 μM, in the perilymph fluid in the inner ear and VPA is administered in amount sufficient to achieve a concentration of about is about 100 μM to 4 mM in the perilymph fluid in the inner ear. Alternatively, the E11 to a subject, for example to the middle ear at a concentration of about 0.1 mM, 0.2 mM, 0.3 mM, 0.4 mM, 0.5 mM, 0.6 mM, 0.7 mM, 0.8 mM, 0.9 mM, 1 mM, 2 mM, 3 mM, 4 mM, 5 mM, 6 mM, 7 mM, 8 mM, 9 mM, 10 mM, 12 mM, 14 mM, 16 mM, 18 mM, or 20 mM and CHIR99021 is administered to a subject, for example to the middle ear at a concentration of about 1 mM, 2 mM, 3 mM, 4 mM, 5 mM, 6 mM, 7 mM, 8 mM, 9 mM, or 10 mM and VPA is administered to a subject, for example to the middle ear at a concentration about 100 mM to 4,000 mM.

In some embodiments the EZH2 inhibitor is PF-06821497 and the Wnt agonist is AZD1080 and the second epigenetic agent is VPA. In some embodiments, PF-06821497 is administered, in amount sufficient to achieve a concentration of about 0.1 μM, 0.2 μM, 0.3 μM, 0.4 uM, 0.5 μM, 0.6 μM, 0.7 μM, 0.8 μM, 0.9 μM, 1 μM, 2 μM, 3 μM, 4 μM, 5 μM, 6 μM, 7 μM, 8 μM, 9 μM, or 10 μM in the perilymph fluid in the inner ear and AZD1080 is administered, in amount sufficient to achieve a concentration of about is about 1 μM, 2 μM, 3 μM, 4 μM, 5 μM, 6 μM, 7 μM, 8 μM, 9 μM, or 10 μM in the perilymph fluid in the inner ear and VPA is administered in amount sufficient to achieve a concentration of about is about 100 μM to 4 mM in the perilymph fluid in the inner ear. Alternatively, PF-06821497 is administered to a subject, for example to the middle ear at a concentration of about 0.1 mM, 0.2 mM, 0.3 mM, 0.4 mM, 0.5 mM, 0.6 mM, 0.7 mM, 0.8 mM, 0.9 mM, 1 mM, 2 mM, 3 mM, 4 mM, 5 mM, 6 mM, 7 mM, 8 mM, 9 mM, or 10 mM and AZD1080, and is administered to a subject, for example to the middle ear at a concentration of about 1 mM, 2 mM, 3 mM, 4 mM, 5 mM, 6 mM, 7 mM, 8 mM, 9 mM, or 10 mM and VPA is administered to a subject, for example to the middle ear at a concentration about 100 mM to 4,000 mM.

In some embodiments the EZH2 inhibitor is PF-06821497 and the Wnt agonist is LY209031 and the second epigenetic agent VPA. In some embodiments, PF-06821497 is administered, in amount sufficient to achieve a concentration of about 0.1 μM, 0.2 μM, 0.3 μM, 0.4 uM, 0.5 μM, 0.6 μM, 0.7 μM, 0.8 μM, 0.9 μM, 1 μM, 2 μM, 3 μM, 4 μM, 5 μM, 6 μM, 7 μM, 8 μM, 9 μM, or 10 μM in the perilymph fluid in the inner ear and LY2090314 is administered, in amount sufficient to achieve a concentration of about 1 nM, 5 nM, 10 nM, 15 nM, 20 nM or 40 nM, in the perilymph fluid in the inner ear and VPA is administered in amount sufficient to achieve a concentration of about is about 100 μM to 4 mM in the perilymph fluid in the inner ear. Alternatively, PF-06821497 is administered to a subject, for example to the middle ear at a concentration of about 0.1 mM, 0.2 mM, 0.3 mM, 0.4 mM, 0.5 mM, 0.6 mM, 0.7 mM, 0.8 mM, 0.9 mM, 1 mM, 2 mM, 3 mM, 4 mM, 5 mM, 6 mM, 7 mM, 8 mM, 9 mM, or 10 mM and LY2090314, and is administered to a subject, for example to the middle ear at a concentration of about 1 μM, 5 μM, 10 μM, 15 μM, 20 μM, or 40 μM and VPA is administered to a subject, for example to the middle ear at a concentration about 100 mM to 4,000 mM.

In some embodiments the EZH2 inhibitor is PF-06821497 and the Wnt agonist is a substituted 3-Imidazo[1,2-a]pyridin-3-yl-4-(1,2,3,4-tetrahydro-[1,4]diazepino-[6,7,1-hi]indol-7-yl)pyrrole-2,5-dione and the second epigenetic agent is VPA. In some embodiments, PF-06821497 is administered, in amount sufficient to achieve a concentration of about 0.1 μM, 0.2 μM, 0.3 μM, 0.4 uM, 0.5 μM, 0.6 μM, 0.7 μM, 0.8 μM, 0.9 μM, 1 μM, 2 μM, 3 μM, 4 μM, 5 μM, 6 μM, 7 μM, 8 μM, 9 μM, or 10 μM in the perilymph fluid in the inner ear and the substituted 3-Imidazo[1,2-a]pyridin-3-yl-4-(1,2,3,4-tetrahydro-[1,4]diazepino-[6,7,1-hi]indol-7-yl)pyrrole-2,5-dione is administered, in amount sufficient to achieve a concentration of about 1 nM, 5 nM, 10 nM, 15 nM, 20 nM, 50 nM, 100 nM, 250 nM, or 500 nM, in the perilymph fluid in the inner ear and VPA is administered in amount sufficient to achieve a concentration of about is about 100 μM to 4 mM in the perilymph fluid in the inner ear. Alternatively, PF-06821497 is administered to a subject, for example to the middle ear at a concentration of about 0.1 mM, 0.2 mM, 0.3 mM, 0.4 mM, 0.5 mM, 0.6 mM, 0.7 mM, 0.8 mM, 0.9 mM, 1 mM, 2 mM, 3 mM, 4 mM, 5 mM, 6 mM, 7 mM, 8 mM, 9 mM, or 10 mM and the substituted 3-Imidazo[1,2-a]pyridin-3-yl-4-(1,2,3,4-tetrahydro-[1,4]diazepino-[6,7,1-hi]indol-7-yl)pyrrole-2,5-dione and is administered to a subject, for example to the middle ear at a concentration of about 1 μM, 5 μM, 10 μM, 15 μM, 20 μM, 50 μM, 100 μM, 250 μM or 500 μM and VPA is administered to a subject, for example to the middle ear at a concentration about 100 mM to 4,000 mM.

In some embodiments the EZH2 inhibitor is PF-06821497 and the Wnt agonist is GSK3 inhibitor XXII and the second epigenetic agent is VPA. In some embodiments, PF-06821497 is administered, in amount sufficient to achieve a concentration of about 0.1 μM, 0.2 μM, 0.3 μM, 0.4 uM, 0.5 μM, 0.6 μM, 0.7 μM, 0.8 μM, 0.9 μM, 1 μM, 2 μM, 3 μM, 4 μM, 5 μM, 6 μM, 7 μM, 8 μM, 9 μM, or 10 μM in the perilymph fluid in the inner ear and GSK3-inhibitor XXII is administered, in amount sufficient to achieve a concentration of about 0.1 μM, 0.2 μM, 0.3 μM, 0.4 μM, 0.5 μM, 0.6 μM, 0.7 μM, 0.8 μM, 0.9 μM, or 1.0 μM, in the perilymph fluid in the inner ear and VPA is administered in amount sufficient to achieve a concentration of about is about 100 μM to 4 mM in the perilymph fluid in the inner ear. Alternatively, PF-06821497 is administered to a subject, for example to the middle ear at a concentration of about 0.1 mM, 0.2 mM, 0.3 mM, 0.4 mM, 0.5 mM, 0.6 mM, 0.7 mM, 0.8 mM, 0.9 mM, 1 mM, 2 mM, 3 mM, 4 mM, 5 mM, 6 mM, 7 mM, 8 mM, 9 mM, or 10 mM and the GSK3-inhibitor XXII is administered to a subject, for example to the middle ear at a concentration of about 0.1 mM, 0.2 mM, 0.3 mM, 0.4 mM, 0.5 mM, 0.6 mM, 0.7 mM, 0.8 mM, 0.9 mM, or 1.0 mM and VPA is administered to a subject, for example to the middle ear at a concentration about 100 mM to 4,000 mM.

In some embodiments the EZH2 inhibitor is PF-06821497 and the Wnt agonist is CHIR99021 and the second epigenetic agent is VPA. In some embodiments, PF-06821497 is administered, in amount sufficient to achieve a concentration of about 0.1 μM, 0.2 μM, 0.3 μM, 0.4 μM, 0.5 μM, 0.6 μM, 0.7 μM, 0.8 μM, 0.9 μM, 1 μM, 2 μM, 3 μM, 4 μM, 5 μM, 6 μM, 7 μM, 9 μM, or 10 μM in the perilymph fluid in the inner ear and CHIR99021 is administered, in amount sufficient to achieve a concentration of about 1 μM, 2 μM, 3 μM, 4 μM, 5 μM, 6 μM, 7 μM, 8 μM, 9 μM, or 10 μM, in the perilymph fluid in the inner ear and VPA is administered in amount sufficient to achieve a concentration of about is about 100 μM to 4 mM in the perilymph fluid in the inner ear. Alternatively, PF-06821497 is administered to a subject, for example to the middle ear at a concentration of about 0.1 mM, 0.2 mM, 0.3 mM, 0.4 mM, 0.5 mM, 0.6 mM, 0.7 mM, 0.8 mM, 0.9 mM, 1 mM, 2 mM, 3 mM, 4 mM, 5 mM, 6 mM, 7 mM, 8 mM, 9 mM, or 10 mM and CHIR99021 is administered to a subject, for example to the middle ear at a concentration of about 1 mM, 2 mM, 3 mM, 4 mM, 5 mM, 6 mM, 7 mM, 8 mM, 9 mM, or 10 mM and VPA is administered to a subject, for example to the middle ear at a concentration about 100 mM to 4,000 mM.

In some embodiments the EZH2 inhibitor is valemetostat and the Wnt agonist is AZD1080 and the second epigenetic agent is VPA. In some embodiments, valemetostat is administered, in amount sufficient to achieve a concentration of about 0.1 μM, 0.2 μM, 0.3 μM, 0.4 uM, 0.5 μM, 0.6 μM, 0.7 μM, 0.8 μM, 0.9 μM, 1 μM, 2 μM, 3 μM, 4 μM, 5 μM, 6 μM, 7 μM, 8 μM, 9 μM, or 10 μM in the perilymph fluid in the inner ear and AZD1080 is administered, in amount sufficient to achieve a concentration of about is about 1 μM, 2 μM, 3 μM, 4 μM, 5 μM, 6 μM, 7 μM, 8 μM, 9 μM, or 10 μM in the perilymph fluid in the inner ear and VPA is administered in amount sufficient to achieve a concentration of about is about 100 μM to 4 mM in the perilymph fluid in the inner ear. Alternatively, valemetostat is administered to a subject, for example to the middle ear at a concentration of about 0.1 mM, 0.2 mM, 0.3 mM, 0.4 mM, 0.5 mM, 0.6 mM, 0.7 mM, 0.8 mM, 0.9 mM, 1 mM, 2 mM, 3 mM, 4 mM, 5 mM, 6 mM, 7 mM, 8 mM, 9 mM, or 10 mM and AZD1080, and is administered to a subject, for example to the middle ear at a concentration of about 1 mM, 2 mM, 3 mM, 4 mM, 5 mM, 6 mM, 7 mM, 8 mM, 9 mM, or 10 mM and VPA is administered to a subject, for example to the middle ear at a concentration about 100 mM to 4,000 mM.

In some embodiments the EZH2 inhibitor is valemetostat and the Wnt agonist is LY209031 and the second epigenetic agent is VPA. In some embodiments, valemetostat is administered, in amount sufficient to achieve a concentration of about 0.1 μM, 0.2 μM, 0.3 μM, 0.4 uM, 0.5 μM, 0.6 μM, 0.7 μM, 0.8 μM, 0.9 μM, 1 μM, 2 μM, 3 μM, 4 μM, 5 μM, 6 μM, 7 μM, 8 μM, 9 μM, or 10 μM in the perilymph fluid in the inner ear and LY2090314 is administered, in amount sufficient to achieve a concentration of about 1 nM, 5 nM, 10 nM, 15 nM, 20 nM or 40 nM, in the perilymph fluid in the inner ear and VPA is administered in amount sufficient to achieve a concentration of about is about 100 μM to 4 mM in the perilymph fluid in the inner ear. Alternatively, valemetostat is administered to a subject, for example to the middle ear at a concentration of about 0.1 mM, 0.2 mM, 0.3 mM, 0.4 mM, 0.5 mM, 0.6 mM, 0.7 mM, 0.8 mM, 0.9 mM, 1 mM, 2 mM, 3 mM, 4 mM, 5 mM, 6 mM, 7 mM, 8 mM, 9 mM, or 10 mM and LY2090314, and is administered to a subject, for example to the middle ear at a concentration of about 1 μM, 5 μM, 10 μM, 15 μM, 20 μM, or 40 μM and VPA is administered to a subject, for example to the middle ear at a concentration about 100 mM to 4,000 mM.

In some embodiments the EZH2 inhibitor is valemetostat and the Wnt agonist is a substituted 3-Imidazo[1,2-a]pyridin-3-yl-4-(1,2,3,4-tetrahydro-[1,4]diazepino-[6,7,1-hi]indol-7-yl)pyrrole-2,5-dione and the second epigenetic agent is VPA. In some embodiments, valemetostat is administered, in amount sufficient to achieve a concentration of about 0.1 μM, 0.2 μM, 0.3 μM, 0.4 uM, 0.5 μM, 0.6 μM, 0.7 μM, 0.8 μM, 0.9 μM, 1 μM, 2 μM, 3 μM, 4 μM, 5 μM, 6 μM, 7 μM, 8 μM, 9 μM, or 10 μM in the perilymph fluid in the inner ear and the substituted 3-Imidazo[1,2-a]pyridin-3-yl-4-(1,2,3,4-tetrahydro-[1,4]diazepino-[6,7,1-hi]indol-7-yl)pyrrole-2,5-dione is administered, in amount sufficient to achieve a concentration of about 1 nM, 5 nM, 10 nM, 15 nM, 20 nM, 50 nM, 100 nM, 250 nM, or 500 nM, in the perilymph fluid in the inner ear and VPA is administered in amount sufficient to achieve a concentration of about is about 100 μM to 4 mM in the perilymph fluid in the inner ear. Alternatively, valemetostat is administered to a subject, for example to the middle ear at a concentration of about 0.1 mM, 0.2 mM, 0.3 mM, 0.4 mM, 0.5 mM, 0.6 mM, 0.7 mM, 0.8 mM, 0.9 mM, 1 mM, 2 mM, 3 mM, 4 mM, 5 mM, 6 mM, 7 mM, 8 mM, 9 mM, or 10 mM and the substituted 3-Imidazo[1,2-a]pyridin-3-yl-4-(1,2,3,4-tetrahydro-[1,4]diazepino-[6,7,1-hi]indol-7-yl)pyrrole-2,5-dione and is administered to a subject, for example to the middle ear at a concentration of about 1 μM, 5 μM, 10 μM, 15 μM, 20 μM, 50 μM, 100 μM, 250 μM or 500 μM and VPA is administered to a subject, for example to the middle ear at a concentration about 100 mM to 4,000 mM.

In some embodiments the EZH2 inhibitor is valemetostat and the Wnt agonist is GSK3 inhibitor XXII and the second epigenetic agent is VPA. In some embodiments, valemetostat is administered, in amount sufficient to achieve a concentration of about 0.1 μM, 0.2 μM, 0.3 μM, 0.4 uM, 0.5 μM, 0.6 μM, 0.7 μM, 0.8 μM, 0.9 μM, 1 μM, 2 μM, 3 μM, 4 μM, 5 μM, 6 μM, 7 μM, 8 μM, 9 μM, or 10 μM in the perilymph fluid in the inner ear and GSK3-inhibitor XXII is administered, in amount sufficient to achieve a concentration of about 0.1 μM, 0.2 μM, 0.3 μM, 0.4 μM, 0.5 μM, 0.6 μM, 0.7 μM, 0.8 μM, 0.9 μM, or 1.0 μM, in the perilymph fluid in the inner ear and VPA is administered in amount sufficient to achieve a concentration of about is about 100 μM to 4 mM in the perilymph fluid in the inner ear. Alternatively, valemetostat is administered to a subject, for example to the middle ear at a concentration of about 0.1 mM, 0.2 mM, 0.3 mM, 0.4 mM, 0.5 mM, 0.6 mM, 0.7 mM, 0.8 mM, 0.9 mM, 1 mM, 2 mM, 3 mM, 4 mM, 5 mM, 6 mM, 7 mM, 8 mM, 9 mM, or 10 mM and the GSK3-inhibitor XVII is administered to a subject, for example to the middle ear at a concentration of about 0.1 mM, 0.2 mM, 0.3 mM, 0.4 mM, 0.5 mM, 0.6 mM, 0.7 mM, 0.8 mM, 0.9 mM, or 1.0 mM and VPA is administered to a subject, for example to the middle ear at a concentration about 100 mM to 4,000 mM.

In some embodiments the EZH2 inhibitor is valemetostat and the Wnt agonist is CHIR99021 and the second epigenetic agent is VPA. In some embodiments, valemetostat is administered, in amount sufficient to achieve a concentration of about 0.1 μM, 0.2 μM, 0.3 μM, 0.4 μM, 0.5 μM, 0.6 μM, 0.7 μM, 0.8 μM, 0.9 μM, 1 μM, 2 μM, 3 μM, 4 μM, 5 μM, 6 μM, 7 μM, 8 μM, 9 μM, or 10 μM in the perilymph fluid in the inner ear and CHIR99021 is administered, in amount sufficient to achieve a concentration of about 1 μM, 2 μM, 3 μM, 4 μM, 5 μM, 6 μM, 7 μM, 8 μM, 9 μM, or 10 μM, in the perilymph fluid in the inner ear and VPA is administered in amount sufficient to achieve a concentration of about is about 100 μM to 4 mM in the perilymph fluid in the inner ear. Alternatively, valemetostat is administered to a subject, for example to the middle ear at a concentration of about 0.1 mM, 0.2 mM, 0.3 mM, 0.4 mM, 0.5 mM, 0.6 mM, 0.7 mM, 0.8 mM, 0.9 mM, 1 mM, 2 mM, 3 mM, 4 mM, 5 mM, 6 mM, 7 mM, 8 mM, 9 mM, or 10 mM and CHIR99021 is administered to a subject, for example to the middle ear at a concentration of about 1 mM, 2 mM, 3 mM, 4 mM, 5 mM, 6 mM, 7 mM, 8 mM, 9 mM, or 10 mM and VPA is administered to a subject, for example to the middle ear at a concentration about 100 mM to 4,000 mM.

In some embodiments the EZH2 inhibitor is tazemetostat and the Wnt agonist is AZD1080 and the second epigenetic agent is VPA. In some embodiments, tazemetostat is administered, in amount sufficient to achieve a concentration of about 0.1 μM, 0.2 μM, 0.3 μM, 0.4 uM, 0.5 μM, 0.6 μM, 0.7 μM, 0.8 μM, 0.9 μM, 1 μM, 2 μM, 3 μM, 4 μM, 5 μM, 6 μM, 7 μM, 8 μM, 9 μM, or 10 μM in the perilymph fluid in the inner ear and AZD1080 is administered, in amount sufficient to achieve a concentration of about is about 1 μM, 2 μM, 3 μM, 4 μM, 5 μM, 6 μM, 7 μM, 8 μM, 9 μM, or 10 μM in the perilymph fluid in the inner ear and VPA is administered in amount sufficient to achieve a concentration of about is about 100 μM to 4 mM in the perilymph fluid in the inner ear. Alternatively, tazemetostat is administered to a subject, for example to the middle ear at a concentration of about 0.1 mM, 0.2 mM, 0.3 mM, 0.4 mM, 0.5 mM, 0.6 mM, 0.7 mM, 0.8 mM, 0.9 mM, 1 mM, 2 mM, 3 mM, 4 mM, 5 mM, 6 mM, 7 mM, 8 mM, 9 mM, or 10 mM and AZD1080, and is administered to a subject, for example to the middle ear at a concentration of about 1 mM, 2 mM, 3 mM, 4 mM, 5 mM, 6 mM, 7 mM, 8 mM, 9 mM, or 10 mM and VPA is administered to a subject, for example to the middle ear at a concentration about 100 mM to 4,000 mM.

In some embodiments the EZH2 inhibitor is tazemetostat and the Wnt agonist is LY209031 and the second epigenetic agent is VPA. In some embodiments, tazemetostat is administered, in amount sufficient to achieve a concentration of about 0.1 μM, 0.2 μM, 0.3 μM, 0.4 uM, 0.5 μM, 0.6 μM, 0.7 μM, 0.8 μM, 0.9 μM, 1 μM, 2 μM, 3 μM, 4 μM, 5 μM, 6 μM, 7 μM, 8 μM, 9 μM, or 10 μM in the perilymph fluid in the inner ear and LY2090314 is administered, in amount sufficient to achieve a concentration of about 1 nM, 5 nM, 10 nM, 15 nM, 20 nM or 40 nM, in the perilymph fluid in the inner ear and VPA is administered in amount sufficient to achieve a concentration of about is about 100 μM to 4 mM in the perilymph fluid in the inner ear. Alternatively, tazemetostat is administered to a subject, for example to the middle ear at a concentration of about 0.1 mM, 0.2 mM, 0.3 mM, 0.4 mM, 0.5 mM, 0.6 mM, 0.7 mM, 0.8 mM, 0.9 mM, 1 mM, 2 mM, 3 mM, 4 mM, 5 mM, 6 mM, 7 mM, 8 mM, 9 mM, or 10 mM and LY2090314, and is administered to a subject, for example to the middle ear at a concentration of about 1 μM, 5 μM, 10 μM, 15 μM, 20 μM, or 40 μM and VPA is administered to a subject, for example to the middle ear at a concentration about 100 mM to 4,000 mM.

In some embodiments the EZH2 inhibitor is tazemetostat and the Wnt agonist is a substituted 3-Imidazo[1,2-a]pyridin-3-yl-4-(1,2,3,4-tetrahydro-[1,4]diazepino-[6,7,1-hi]indol-7-yl)pyrrole-2,5-dione and the second epigenetic agent is VPA. In some embodiments, tazemetostat is administered, in amount sufficient to achieve a concentration of about 0.1 μM, 0.2 μM, 0.3 μM, 0.4 uM, 0.5 μM, 0.6 μM, 0.7 μM, 0.8 μM, 0.9 μM, 1 μM, 2 μM, 3 μM, 4 μM, 5 μM, 6 μM, 7 μM, 8 μM, 9 μM, or 10 μM in the perilymph fluid in the inner ear and the substituted 3-Imidazo[1,2-a]pyridin-3-yl-4-(1,2,3,4-tetrahydro-[1,4]diazepino-[6,7,1-hi]indol-7-yl)pyrrole-2,5-dione is administered, in amount sufficient to achieve a concentration of about 1 nM, 5 nM, 10 nM, 15 nM, 20 nM, 50 nM, 100 nM, 250 nM, or 500 nM, in the perilymph fluid in the inner ear and VPA is administered in amount sufficient to achieve a concentration of about is about 100 μM to 4 mM in the perilymph fluid in the inner ear. Alternatively, tazemetostat is administered to a subject, for example to the middle ear at a concentration of about 0.1 mM, 0.2 mM, 0.3 mM, 0.4 mM, 0.5 mM, 0.6 mM, 0.7 mM, 0.8 mM, 0.9 mM, 1 mM, 2 mM, 3 mM, 4 mM, 5 mM, 6 mM, 7 mM, 8 mM, 9 mM, or 10 mM and the substituted 3-Imidazo[1,2-a]pyridin-3-yl-4-(1,2,3,4-tetrahydro-[1,4]diazepino-[6,7,1-hi]indol-7-yl)pyrrole-2,5-dione and is administered to a subject, for example to the middle ear at a concentration of about 1 μM, 5 μM, 10 μM, 15 μM, 20 μM, 50 μM, 100 μM, 250 μM or 500 μM and VPA is administered to a subject, for example to the middle ear at a concentration about 100 mM to 4,000 mM.

In some embodiments the EZH2 inhibitor is tazemetostat and the Wnt agonist is GSK3 inhibitor XXII and the second epigenetic agent is VPA. In some embodiments, tazemetostat is administered, in amount sufficient to achieve a concentration of about 0.1 μM, 0.2 μM, 0.3 μM, 0.4 uM, 0.5 μM, 0.6 μM, 0.7 μM, 0.8 μM, 0.9 μM, 1 μM, 2 μM, 3 μM, 4 μM, 5 μM, 6 μM, 7 μM, 8 μM, 9 μM, or 10 μM in the perilymph fluid in the inner ear and GSK3-inhibitor XXII is administered, in amount sufficient to achieve a concentration of about 0.1 μM, 0.2 μM, 0.3 μM, 0.4 μM, 0.5 μM, 0.6 μM, 0.7 μM, 0.8 μM, 0.9 μM, or 1.0 μM, in the perilymph fluid in the inner ear and VPA is administered in amount sufficient to achieve a concentration of about is about 100 μM to 4 mM in the perilymph fluid in the inner ear. Alternatively, tazemetostat is administered to a subject, for example to the middle ear at a concentration of about 0.1 mM, 0.2 mM, 0.3 mM, 0.4 mM, 0.5 mM, 0.6 mM, 0.7 mM, 0.8 mM, 0.9 mM, 1 mM, 2 mM, 3 mM, 4 mM, 5 mM, 6 mM, 7 mM, 8 mM, 9 mM, or 10 mM and the GSK3-inhibitor XXII is administered to a subject, for example to the middle ear at a concentration of about 0.1 mM, 0.2 mM, 0.3 mM, 0.4 mM, 0.5 mM, 0.6 mM, 0.7 mM, 0.8 mM, 0.9 mM, or 1.0 mM and VPA is administered to a subject, for example to the middle ear at a concentration about 100 mM to 4,000 mM.

In some embodiments the EZH2 inhibitor is tazemetostat and the Wnt agonist is CHIR99021 and the second epigenetic agent is VPA. In some embodiments, tazemetostat is administered, in amount sufficient to achieve a concentration of about 0.1 μM, 0.2 μM, 0.3 μM, 0.4 μM, 0.5 μM, 0.6 μM, 0.7 μM, 0.8 μM, 0.9 μM, 1 μM, 2 μM, 3 μM, 4 μM, 5 μM, 6 μM, 7 μM, 8 μM, 9 μM, or 10 μM in the perilymph fluid in the inner ear and CHIR99021 is administered, in amount sufficient to achieve a concentration of about 1 μM, 2 μM, 3 μM, 4 μM, 5 μM, 6 μM, 7 μM, 8 μM, 9 μM, or 10 μM, in the perilymph fluid in the inner ear and VPA is administered in amount sufficient to achieve a concentration of about is about 100 μM to 4 mM in the perilymph fluid in the inner ear. Alternatively, tazemetostat is administered to a subject, for example to the middle ear at a concentration of about 0.1 mM, 0.2 mM, 0.3 mM, 0.4 mM, 0.5 mM, 0.6 mM, 0.7 mM, 0.8 mM, 0.9 mM, 1 mM, 2 mM, 3 mM, 4 mM, 5 mM, 6 mM, 7 mM, 8 mM, 9 mM, or 10 mM and CHIR99021 is administered to a subject, for example to the middle ear at a concentration of about 1 mM, 2 mM, 3 mM, 4 mM, 5 mM, 6 mM, 7 mM, 8 mM, 9 mM, or 10 mM and VPA is administered to a subject, for example to the middle ear at a concentration about 100 mM to 4,000 mM.

In some embodiments the EZH2 inhibitor is CPI-360 and the Wnt agonist is AZD1080 and the second epigenetic agent is VPA. In some embodiments, CPI-360 is administered, in amount sufficient to achieve a concentration of about 0.1 nM, 0.2 nM, 0.3 nM, 0.4 nM, 0.5 nM, 0.6 nM, 0.7 nM, 0.8 nM, 0.9 nM, 1.0 nM, 2.0 nM, 3.0 nM, 4.0 nM, 5.0 nM, 6.0 nM, 7.0 nM, 8.0 nM, 9.0 nM, 10 nM, 20 nM, 30 nM, 40 nM, 50 nM, 60 nM, 70 nM, 80 nM, 90 nM, 100 nM, 200 nM, 300 nM, 400 nM, 500 nM, 1 μM, 5 μM, 10 μM, or 50 μM in the perilymph fluid in the inner ear; AZD1080 is administered, in amount sufficient to achieve a concentration of about is about 1 μM, 2 μM, 3 μM, 4 μM, 5 μM, 6 μM, 7 μM, 8 μM, 9 μM, or 10 μM, in the perilymph fluid in the inner ear and VPA is administered in amount sufficient to achieve a concentration of about is about 100 μM to 4 mM in the perilymph fluid in the inner ear. Alternatively, the CPI-360 is administered to a subject, for example to the middle ear at a concentration of about 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 1 mM, 5 mM, 10 mM, or 50 mM and AZD1080, and is administered to a subject, for example to the middle ear at a concentration of about 1 mM, 2 mM, 3 mM, 4 mM, 5 mM, 6 mM, 7 mM, 8 mM, 9 mM, or 10 mM and VPA is administers to a subject, for example to the middle ear at a concentration about 100 mM to 4,000 mM.

In some embodiments the EZH2 inhibitor is CPI-360 and the Wnt agonist is LY2090314 and the second epigenetic agent is VPA. In some embodiments, CPI-360 is administered, in amount sufficient to achieve a concentration of about 0.1 nM, 0.2 nM, 0.3 nM, 0.4 nM, 0.5 nM, 0.6 nM, 0.7 nM, 0.8 nM, 0.9 nM, 1.0 nM, 2.0 nM, 3.0 nM, 4.0 nM, 5.0 nM, 6.0 nM, 7.0 nM, 8.0 nM, 9.0 nM, 10 nM, 20 nM, 30 nM, 40 nM, 50 nM, 60 nM, 70 nM, 80 nM, 90 nM, 100 nM, 200 nM, 300 nM, 400 nM, 500 nM, 1 μM, 5 μM, 10 μM, or 50 μM in the perilymph fluid in the inner ear; LY2090314 is administered, in amount sufficient to achieve a concentration of about 1 nM, 5 nM, 10 nM, 15 nM, 20 nM or 40 nM in the perilymph fluid in the inner ear and VPA is administered in amount sufficient to achieve a concentration of about is about 100 μM to 4 mM in the perilymph fluid in the inner ear. Alternatively, the CPI-360 is administered to a subject, for example to the middle ear at a concentration of about 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 1 mM, 5 mM, 10 mM, or 50 mM and LY2090314, and is administered to a subject, for example to the middle ear at a concentration of about 1 μM, 5 μM, 10 μM, 15 μM, 20 μM or 40 μM and VPA is administers to a subject, for example to the middle ear at a concentration about 100 mM to 4,000 mM.

In some embodiments the EZH2 inhibitor is CPI-360 and the Wnt agonist is a substituted 3-imidazo[1,2-a]pyridin-3-yl-4-(1,2,3,4-tetrahydro-[1,4]diazepino-[6,7,1-hi]indol-7-yl)pyrrole-2,5-dione and the second epigenetic agent is VPA. In some embodiments, CPI-360 is administered, in amount sufficient to achieve a concentration of about 0.1 nM, 0.2 nM, 0.3 nM, 0.4 nM, 0.5 nM, 0.6 nM, 0.7 nM, 0.8 nM, 0.9 nM, 1.0 nM, 2.0 nM, 3.0 nM, 4.0 nM, 5.0 nM, 6.0 nM, 7.0 nM, 8.0 nM, 9.0 nM, 10 nM, 20 nM, 30 nM, 40 nM, 50 nM, 60 nM, 70 nM, 80 nM, 90 nM, 100 nM, 200 nM, 300 nM, 400 nM, 500 nM, 1 μM, 5 μM, 10 μM, or 50 μM in the perilymph fluid in the inner ear; the substituted 3-Imidazo[1,2-a]pyridin-3-yl-4-(1,2,3,4-tetrahydro-[1,4]diazepino-[6,7,1-hi]indol-7-yl)pyrrole-2,5-dione is administered, in amount sufficient to achieve a concentration of about 1 nM, 5 nM, 10 nM, 15 nM, 20 nM, 50 nM, 100 nM, 250 nM, or 500 nM, in the perilymph fluid in the inner ear and VPA is administered in amount sufficient to achieve a concentration of about is about 100 μM to 4 mM in the perilymph fluid in the inner ear. Alternatively, the CPI-360 is administered to a subject, for example to the middle ear at a concentration of about 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 1 mM, 5 mM, 10 mM, or 50 mM and the substituted 3-Imidazo[1,2-a]pyridin-3-yl-4-(1,2,3,4-tetrahydro-[1,4]diazepino-[6,7,1-hi]indol-7-yl)pyrrole-2,5-dione and is administered to a subject, for example to the middle ear at a concentration of about 1 μM, 5 μM, 10 μM, 15 μM, 20 μM, 50 μM, 100 μM, 250 μM, or 500 μM and VPA is administers to a subject, for example to the middle ear at a concentration about 100 mM to 4,000 mM.

In some embodiments the EZH2 inhibitor is CPI-360 and the Wnt agonist is GSK3 inhibitor XXII and the second epigenetic agent is VPA. In some embodiments, CPI-360 is administered, in amount sufficient to achieve a concentration of about 0.1 nM, 0.2 nM, 0.3 nM, 0.4 nM, 0.5 nM, 0.6 nM, 0.7 nM, 0.8 nM, 0.9 nM, 1.0 nM, 2.0 nM, 3.0 nM, 4.0 nM, 5.0 nM, 6.0 nM, 7.0 nM, 8.0 nM, 9.0 nM, 10 nM, 20 nM, 30 nM, 40 nM, 50 nM, 60 nM, 70 nM, 80 nM, 90 nM, 100 nM, 200 nM, 300 nM, 400 nM, 500 nM, 1 μM, 5 μM, 10 μM, or 50 μM in the perilymph fluid in the inner ear; GSK3-inhibitor XXII is administered, in amount sufficient to achieve a concentration of about 0.1 μM, 0.2 μM, 0.3 μM, 0.4 μM, 0.5 μM, 0.6 μM, 0.7 μM, 0.8 μM, 0.9 μM, or 1.0 μM, in the perilymph fluid in the inner ear and VPA is administered in amount sufficient to achieve a concentration of about is about 100 μM to 4 mM in the perilymph fluid in the inner ear. Alternatively, the CPI-360 is administered to a subject, for example to the middle ear at a concentration of about 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM 1 mM, 5 mM, 10 mM, or 50 mM and the GSK3-inhibitor XVII is administered to a subject, for example to the middle ear at a concentration about 0.1 mM, 0.2 mM, 0.3 mM, 0.4 mM, 0.5 mM, 0.6 mM, 0.7 mM, 0.8 mM, 0.9 mM, or 1.0 mM and VPA is administered to a subject, for example to the middle ear at a concentration about 100 mM to 4,000 mM.

In some embodiments the EZH2 inhibitor is CPI-360 and the Wnt agonist is CHIR99021 and the second epigenetic agent is VPA. In some embodiments, CPI-360 is administered, in amount sufficient to achieve a concentration of about 0.1 nM, 0.2 nM, 0.3 nM, 0.4 nM, 0.5 nM, 0.6 nM, 0.7 nM, 0.8 nM, 0.9 nM, 1.0 nM, 2.0 nM, 3.0 nM, 4.0 nM, 5.0 nM, 6.0 nM, 7.0 nM, 8.0 nM, 9.0 nM, 10 nM, 2.0 nM, 30 nM, 40 nM, 50 nM, 60 nM, 70 nM, 80 nM, 90 nM, 100 nM, 200 nM, 300 nM, 400 nM, 500 nM, 1 μM, 5 μM, 10 μM, or 50 μM in the perilymph fluid in the inner ear; CHIR99021 is administered, in amount sufficient to achieve a concentration of about 1 μM, 2 μM, 3 μM, 4 μM, 5 μM, 6 μM, 7 μM, 8 μM, 9 μM, or 10 μM, in the perilymph fluid in the inner ear and VPA is administered in amount sufficient to achieve a concentration of about is about 100 μM to 4 mM in the perilymph fluid in the inner ear. Alternatively, the CPI-360 is administered to a subject, for example to the middle ear at a concentration of about 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 1 mM, 5 mM, 10 mM, or 50 mM and CHIR99021 is administered to a subject, for example to the middle ear at a concentration of about 1 mM, 2 mM, 3 mM, 4 mM, 5 mM, 6 mM, 7 mM, 8 mM, 9 mM, or 10 mM and VPA is administered to a subject, for example to the middle ear at a concentration about 100 mM to 4,000 mM.

In some embodiments the EZH12 inhibitor is EPZ011989 and the Wnt agonist is AZD1080 and the second epigenetic agent is VPA. In some embodiments, EPZ011989 is administered, in amount sufficient to achieve a concentration of about 0.1 nM, 0.2 nM, 0.3 nM, 0.4 nM, 0.5 nM, 0.6 nM, 0.7 nM, 0.8 nM, 0.9 nM, 1.0 nM, 2.0 nM, 3.0 nM, 4.0 nM, 5.0 nM, 6.0 nM, 7.0 nM, 8.0 nM, 9.0 nM, 10 nM, 20 nM, 30 nM, 40 nM, 50 nM, 60 nM, 70 nM, 80 nM, 90 nM, 100 nM, 200 nM, 300 nM, 400 nM, 500 nM, 1 μM, 5 μM, 10 μM, or 50 μM in the perilymph fluid in the inner ear; AZD1080 is administered, in amount sufficient to achieve a concentration of about is about 1 μM, 2 μM, 3 μM, 4 μM, 5 μM, 6 μM, 7 μM, 8 μM, 9 μM, or 10 μM, in the perilymph fluid in the inner ear and VPA is administered in amount sufficient to achieve a concentration of about is about 100 μM to 4 mM in the perilymph fluid in the inner ear. Alternatively, the EPZ011989 is administered to a subject, for example to the middle ear at a concentration of about 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 1 mM, 5 mM, 10 mM, or 50 mM and AZD1080, and is administered to a subject, for example to the middle ear at a concentration of about 1 mM, 2 mM, 3 mM, 4 mM, 5 mM, 6 mM, 7 mM, 8 mM, 9 mM, or 10 mM and VPA is administers to a subject, for example to the middle ear at a concentration about 100 mM to 4,000 mM.

In some embodiments the EZH2 inhibitor is EPZ011989 and the Wnt agonist is LY2090314 and the second epigenetic agent is VPA. In some embodiments, EPZ011989 is administered, in amount sufficient to achieve a concentration of about 0.1 nM, 0.2 nM, 0.3 nM, 0.4 nM, 0.5 nM, 0.6 nM, 0.7 nM, 0.8 nM, 0.9 nM, 1.0 nM, 2.0 nM, 3.0 nM, 4.0 nM, 5.0 nM, 6.0 nM, 7.0 nM, 8.0 nM, 9.0 nM, 10 nM, 20 nM, 30 nM, 40 nM, 50 nM, 60 nM, 70 nM, 80 nM, 90 nM, 100 nM, 200 nM, 300 nM, 400 nM, 500 nM, 1 μM, 5 μM, 10 μM, or 50 μM in the perilymph fluid in the inner ear; LY2090314 is administered, in amount sufficient to achieve a concentration of about 1 nM, 5 nM, 10 nM, 15 nM, 20 nM or 40 nM in the perilymph fluid in the inner ear and VPA is administered in amount sufficient to achieve a concentration of about is about 100 μM to 4 mM in the perilymph fluid in the inner ear. Alternatively, the EPZ011989 is administered to a subject, for example to the middle ear at a concentration of about 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 1 mM, 5 mM, 10 mM, or 50 mM and LY2090314, and is administered to a subject, for example to the middle ear at a concentration of about 1 μM, 5 μM, 10 μM, 15 μM, 20 μM or 40 μM and VPA is administers to a subject, for example to the middle ear at a concentration about 100 mM to 4,000 mM.

In some embodiments the EZH2 inhibitor is EPZ011989 and the Wnt agonist is a substituted 3-Imidazo[1,2-a]pyridin-3-yl-4-(1,2,3,4-tetrahydro-[1,4]diazepino-[6,7,1-hi]indol-7-yl)pyrrole-2,5-dione and the second epigenetic agent is VPA. In some embodiments, EPZ011989 is administered, in amount sufficient to achieve a concentration of about 0.1 nM, 0.2 nM, 0.3 nM, 0.4 nM, 0.5 nM, 0.6 nM, 0.7 nM, 0.8 nM, 0.9 nM, 1.0 nM, 2.0 nM, 3.0 nM, 4.0 nM, 5.0 nM, 6.0 nM, 7.0 nM, 8.0 nM, 9.0 nM, 10 nM, 20 nM, 30 nM, 40 nM, 50 nM, 60 nM, 70 nM, 80 nM, 90 nM, 100 nM, 200 nM, 300 nM, 400 nM, 500 nM, 1 μM, 5 μM, 10 μM, or 50 μM in the perilymph fluid in the inner ear; the substituted 3-Imidazo[1,2-a]pyridin-3-yl-4-(1,2,3,4-tetrahydro-[1,4]diazepino-[6,7,1-hi]indol-7-yl)pyrrole-2,5-dione is administered, in amount sufficient to achieve a concentration of about 1 nM, 5 nM, 10 nM, 15 nM, 20 nM, 50 nM, 100 nM, 250 nM, or 500 nM, in the perilymph fluid in the inner ear and VPA is administered in amount sufficient to achieve a concentration of about is about 100 μM to 4 mM in the perilymph fluid in the inner ear. Alternatively, the EPZ011989 is administered to a subject, for example to the middle ear at a concentration of about 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 1 mM, 5 mM, 10 mM, or 50 mM and the substituted 3-Imidazo[1,2-a]pyridin-3-yl-4-(1,2,3,4-tetrahydro-[1,4]diazepino-[6,7,1-hi]indol-7-yl)pyrrole-2,5-dione and is administered to a subject, for example to the middle ear at a concentration of about 1 μM, 5 μM, 10 μM, 15 μM, 20 μM, 50 μM, 100 μM, 250 μM, or 500 μM and VPA is administers to a subject, for example to the middle ear at a concentration about 100 mM to 4,000 mM.

In some embodiments the EZH2 inhibitor is EPZ011989 and the Wnt agonist is GSK3 inhibitor XXII and the second epigenetic agent is VPA. In some embodiments, EPZ011989 is administered, in amount sufficient to achieve a concentration of about 0.1 nM, 0.2 nM, 0.3 nM, 0.4 nM, 0.5 nM, 0.6 nM, 0.7 nM, 0.8 nM, 0.9 nM, 1.0 nM, 2.0 nM, 3.0 nM, 4.0 nM, 5.0 nM, 6.0 nM, 7.0 nM, 8.0 nM, 9.0 nM, 10 nM, 20 nM, 30 nM, 40 nM, 50 nM, 60 nM, 70 nM, 80 nM, 90 nM, 100 nM, 200 nM, 300 nM, 400 nM, 500 nM, 1 μM, 5 μM, 10 μM, or 50 μM in the perilymph fluid in the inner ear; GSK3-inhibitor XXII is administered, in amount sufficient to achieve a concentration of about 0.1 μM, 0.2 μM, 0.3 μM, 0.4 μM, 0.5 μM, 0.6 μM, 0.7 μM, 0.8 μM, 0.9 μM, or 1.0 μM, in the perilymph fluid in the inner ear and VPA is administered in amount sufficient to achieve a concentration of about is about 100 μM to 4 mM in the perilymph fluid in the inner ear. Alternatively, the EPZ011989 is administered to a subject, for example to the middle ear at a concentration of about 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM 1 mM, 5 mM, 10 mM, or 50 mM and the GSK3-inhibitor XXII is administered to a subject, for example to the middle ear at a concentration about 0.1 mM, 0.2 mM, 0.3 mM, 0.4 mM, 0.5 mM, 0.6 mM, 0.7 mM, 0.8 mM, 0.9 mM, or 1.0 mM and VPA is administered to a subject, for example to the middle ear at a concentration about 100 mM to 4,000 mM.

In some embodiments the EZH2 inhibitor is EPZ011989 and the Wnt agonist is CHIR99021 and the second epigenetic agent is VPA. In some embodiments, EPZ011989 is administered, in amount sufficient to achieve a concentration of about 0.1 nM, 0.2 nM, 0.3 nM, 0.4 nM, 0.5 nM, 0.6 nM, 0.7 nM, 0.8 nM, 0.9 nM, 1.0 nM, 2.0 nM, 3.0 nM, 4.0 nM, 5.0 nM, 6.0 nM, 7.0 nM, 8.0 nM, 9.0 nM, 10 nM, 20 nM, 30 nM, 40 nM, 50 nM, 60 nM, 70 nM, 80 nM, 90 nM, 100 nM, 200 nM, 300 nM, 400 nM, 500 nM, 1 μM, 5 μM, 10 μM, or 50 μM in the perilymph fluid in the inner ear; CHIR99021 is administered, in amount sufficient to achieve a concentration of about 1 μM, 2 μM, 3 μM, 4 μM, 5 μM, 6 μM, 7 μM, 8 μM, 9 μM, or 10 μM, in the perilymph fluid in the inner ear and VPA is administered in amount sufficient to achieve a concentration of about is about 100 μM to 4 mM in the perilymph fluid in the inner ear. Alternatively, the EPZ011989 is administered to a subject, for example to the middle ear at a concentration of about 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 1 mM, 5 mM, 10 mM, or 50 mM and CHIR99021 is administered to a subject, for example to the middle ear at a concentration of about 1 mM, 2 mM, 3 mM, 4 mM, 5 mM, 6 mM, 7 mM, 8 mM, 9 mM, or 10 mM and VPA is administered to a subject, for example to the middle ear at a concentration about 100 mM to 4,000 mM.

In some embodiments the EZH2 inhibitor is UNC 2399 and the Wnt agonist is AZD1080 and the second epigenetic agent is VPA. In some embodiments, UNC 2399 is administered, in amount sufficient to achieve a concentration of about 0.1 nM, 0.2 nM, 0.3 nM, 0.4 nM, 0.5 nM, 0.6 nM, 0.7 nM, 0.8 nM, 0.9 nM, 1.0 nM, 2.0 nM, 3.0 nM, 4.0 nM, 5.0 nM, 6.0 nM, 7.0 nM, 8.0 nM, 9.0 nM, 10 nM, 20 nM, 30 nM, 40 nM, 50 nM, 60 nM, 70 nM, 80 nM, 90 nM, 100 nM, 200 nM, 300 nM, 400 nM, 500 nM, 1 μM, 5 μM, 10 μM, or 50 μM in the perilymph fluid in the inner ear; AZD1080 is administered, in amount sufficient to achieve a concentration of about is about 1 μM, 2 μM, 3 μM, 4 μM, 5 μM, 6 μM, 7 μM, 8 μM, 9 μM, or 10 μM, in the perilymph fluid in the inner ear and VPA is administered in amount sufficient to achieve a concentration of about is about 100 μM to 4 mM in the perilymph fluid in the inner ear. Alternatively, the UNC 2399 is administered to a subject, for example to the middle ear at a concentration of about 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 1 mM, 5 mM, 10 mM, or 50 mM and AZD1080, and is administered to a subject, for example to the middle ear at a concentration of about 1 mM, 2 mM, 3 mM, 4 mM, 5 mM, 6 mM, 7 mM, 8 mM, 9 mM, or 10 mM and VPA is administers to a subject, for example to the middle ear at a concentration about 100 mM to 4,000 mM.

In some embodiments the EZH2 inhibitor is UNC 2399 and the Wnt agonist is LY2090314 and the second epigenetic agent is VPA. In some embodiments, UNC 2399 is administered, in amount sufficient to achieve a concentration of about 0.1 nM, 0.2 nM, 0.3 nM, 0.4 nM, 0.5 nM, 0.6 nM, 0.7 nM, 0.8 nM, 0.9 nM, 1.0 nM, 2.0 nM, 3.0 nM, 4.0 nM, 5.0 nM, 6.0 nM, 7.0 nM, 8.0 nM, 9.0 nM, 10 nM, 20 nM, 30 nM, 40 nM, 50 nM, 60 nM, 70 nM, 80 nM, 90 nM, 100 nM, 200 nM, 300 nM, 400 nM, 500 nM, 1 μM, 5 μM, 10 μM, or 50 μM in the perilymph fluid in the inner ear; LY2090314 is administered, in amount sufficient to achieve a concentration of about 1 nM, 5 nM, 10 nM, 15 nM, 20 nM or 40 nM in the perilymph fluid in the inner ear and VPA is administered in amount sufficient to achieve a concentration of about is about 100 μM to 4 mM in the perilymph fluid in the inner ear. Alternatively, the UNC 2399 is administered to a subject, for example to the middle ear at a concentration of about 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 1 mM, 5 mM, 10 mM, or 50 mM and LY2090314, and is administered to a subject, for example to the middle ear at a concentration of about 1 μM, 5 μM, 10 μM, 15 μM, 20 μM or 40 μM and VPA is administers to a subject, for example to the middle ear at a concentration about 100 mM to 4,000 mM.

In some embodiments the EZH2 inhibitor is UNC 2399 and the Wnt agonist is a substituted 3-Imidazo[1,2-a]pyridin-3-yl-4-(1,2,3,4-tetrahydro-[1,4]diazepino-[6,7,1-hi]indol-7-yl)pyrrole-2,5-dione and the second epigenetic agent is VPA In some embodiments, UNC 2399 is administered, in amount sufficient to achieve a concentration of about 0.1 nM, 0.2 nM, 0.3 nM, 0.4 nM, 0.5 nM, 0.6 nM, 0.7 nM, 0.8 nM, 0.9 nM, 1.0 nM, 2.0 nM, 3.0 nM, 4.0 nM, 5.0 nM, 6.0 nM, 7.0 nM, 8.0 nM, 9.0 nM, 10 nM, 20 nM, 30 nM, 40 nM, 50 nM, 60 nM, 70 nM, 80 nM, 90 nM, 100 nM, 200 nM, 300 nM, 400 nM, 500 nM, 1 μM, 5 μM, 10 μM, or 50 μM in the perilymph fluid in the inner ear; the substituted 3-Imidazo[1,2-a]pyridin-3-yl-4-(1,2,3,4-tetrahydro-[1,4]diazepino-[6,7,1-hi]indol-7-yl)pyrrole-2,5-dione is administered, in amount sufficient to achieve a concentration of about 1 nM, 5 nM, 10 nM, 15 nM, 20 nM, 50 nM, 100 nM, 250 nM, or 500 nM, in the perilymph fluid in the inner ear and VPA is administered in amount sufficient to achieve a concentration of about is about 100 μM to 4 mM in the perilymph fluid in the inner ear. Alternatively, the UNC 2399 is administered to a subject, for example to the middle ear at a concentration of about 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 1 mM, 5 mM, 10 mM, or 50 mM and the substituted 3-Imidazo[1,2-a]pyridin-3-yl-4-(1,2,3,4-tetrahydro-[1,4]diazepino-[6,7,1-hi]indol-7-yl)pyrrole-2,5-dione and is administered to a subject, for example to the middle ear at a concentration of about 1 μM, 5 μM, 10 μM, 15 μM, 20 μM, 50 μM, 100 μM, 250 μM, or 500 μM and VPA is administers to a subject, for example to the middle ear at a concentration about 100 mM to 4,000 mM.

In some embodiments the EZH2 inhibitor is UNC 2399 and the Wnt agonist is GSK3 inhibitor XXII and the second epigenetic agent is VPA. In some embodiments, UNC 2399 is administered, in amount sufficient to achieve a concentration of about 0.1 nM, 0.2 nM, 0.3 nM, 0.4 nM, 0.5 nM, 0.6 nM, 0.7 nM, 0.8 nM, 0.9 nM, 1.0 nM, 2.0 nM, 3.0 nM, 4.0 nM, 5.0 nM, 6.0 nM, 7.0 nM, 8.0 nM, 9.0 nM, 10 nM, 2.0 nM, 30 nM, 40 nM, 50 nM, 60 nM, 70 nM, 80 nM, 90 nM, 100 nM, 200 nM, 300 nM, 400 nM, 500 nM, 1 μM, 5 μM, 10 μM, or 50 μM in the perilymph fluid in the inner ear; GSK3-inhibitor XXII is administered, in amount sufficient to achieve a concentration of about 0.1 μM, 0.2 μM, 0.3 μM, 0.4 μM, 0.5 μM, 0.6 μM, 0.7 μM, 0.8 μM, 0.9 μM, or 1.0 μM, in the perilymph fluid in the inner ear and VPA is administered in amount sufficient to achieve a concentration of about is about 100 μM to 4 mM in the perilymph fluid in the inner ear. Alternatively, the UNC 2399 is administered to a subject, for example to the middle ear at a concentration of about 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM 1 mM, 5 mM, 10 mM, or 50 mM and the GSK3-inhibitor XXII is administered to a subject, for example to the middle ear at a concentration about 0.1 mM, 0.2 mM, 0.3 mM, 0.4 mM, 0.5 mM, 0.6 mM, 0.7 mM, 0.8 mM, 0.9 mM, or 1.0 mM and VPA is administered to a subject, for example to the middle ear at a concentration about 100 mM to 4,000 mM.

In some embodiments the EZH2 inhibitor is UNC 2399 and the Wnt agonist is CHIR990 1 and the second epigenetic agent is VPA. In some embodiments, UNC 2399 is administered, in amount sufficient to achieve a concentration of about 0.1 nM, 0.2 nM, 0.3 nM, 0.4 nM, 0.5 nM, 0.6 nM, 0.7 nM, 0.8 nM, 0.9 nM, 1.0 nM, 2.0 nM, 3.0 nM, 4.0 nM, 5.0 nM, 6.0 nM, 7.0 nM, 8.0 nM, 9.0 nM, 10 nM, 20 nM, 30 nM, 40 nM, 50 nM, 60 nM, 70 nM, 80 nM, 90 nM, 100 nM, 200 nM, 300 nM, 400 nM, 500 nM, 1 μM, 5 μM, 10 μM, or 50 μM in the perilymph fluid in the inner ear; CHIR99021 is administered, in amount sufficient to achieve a concentration of about 1 μM, 2 μM, 3 μM, 4 μM, 5 μM, 6 μM, 7 μM, 8 μM, 9 μM, or 10 μM, in the perilymph fluid in the inner ear and VPA is administered in amount sufficient to achieve a concentration of about is about 100 μM to 4 mM in the perilymph fluid in the inner ear. Alternatively, the UNC 2399 is administered to a subject, for example to the middle ear at a concentration of about 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 1 mM, 5 mM, 10 mM, or 50 mM and CHIR99021 is administered to a subject, for example to the middle ear at a concentration of about 1 mM, 2 mM, 3 mM, 4 mM, 5 mM, 6 mM, 7 mM, 8 mM, 9 mM, or 10 mM and VPA is administered to a subject, for example to the middle ear at a concentration about 100 mM to 4,000 mM.

In some embodiments the EZH2 inhibitor is PF-06726304 and the Wnt agonist is AZD1080 and the second epigenetic agent is VPA. In some embodiments, PF-06726304 is administered, in amount sufficient to achieve a concentration of about 0.1 μM, 0.2 μM, 0.3 μM, 0.4 uM, 0.5 μM, 0.6 μM, 0.7 μM, 0.8 μM, 0.9 μM, 1 μM, 2 μM, 3 μM, 4 μM, 5 μM, 6 μM, 7 μM, 8 μM, 9 μM, or 10 μM in the perilymph fluid in the inner ear and AZD1080 is administered, in amount sufficient to achieve a concentration of about is about 1 μM, 2 μ, 3 μM, 4 μM, 5 μM, 6 μM, 7 μM, 8 μM, 9 μM, or 10 μM in the perilymph fluid in the inner ear and VPA is administered in amount sufficient to achieve a concentration of about is about 100 μM to 4 mM in the perilymph fluid in the inner ear. Alternatively, PF-06726304 is administered to a subject, for example to the middle ear at a concentration of about 0.1 mM, 0.2 mM, 0.3 mM, 0.4 mM, 0.5 mM, 0.6 mM, 0.7 mM, 0.8 mM, 0.9 mM, 1 mM, 2 mM, 3 mM, 4 mM, 5 mM, 6 mM, 7 mM, 8 mM, 9 mM, or 10 mM and AZD1080, and is administered to a subject, for example to the middle ear at a concentration of about 1 mM, 2 mM, 3 mM, 4 mM, 5 mM, 6 mM, 7 mM, 8 mM, 9 mM, or 10 mM and VPA is administered to a subject, for example to the middle ear at a concentration about 100 mM to 4,000 mM.

In some embodiments the EZH2 inhibitor is PF-06726304 and the Wnt agonist is LY209031 and the second epigenetic agent is VPA. In some embodiments, PF-06726304 is administered, in amount sufficient to achieve a concentration of about 0.1 μM, 0.2 μM, 0.3 μM, 0.4 uM, 0.5 μM, 0.6 μM, 0.7 μM, 0.8 μM, 0.9 μM, 1 μM, 2 μM, 3 μM, 4 μM, 5 μM, 6 μM, 7 μM, 8 μM, 9 μM, or 10 μM in the perilymph fluid in the inner ear and LY2090314 is administered, in amount sufficient to achieve a concentration of about 1 nM, 5 nM, 10 nM, 15 nM, 20 nM or 40 nM, in the perilymph fluid in the inner ear and VPA is administered in amount sufficient to achieve a concentration of about is about 100 μM to 4 mM in the perilymph fluid in the inner ear. Alternatively, PF-06726304 is administered to a subject, for example to the middle ear at a concentration of about 0.1 mM, 0.2 mM, 0.3 mM, 0.4 mM, 0.5 mM, 0.6 mM, 0.7 mM, 0.8 mM, 0.9 mM, 1 mM, 2 mM, 3 mM, 4 mM, 5 mM, 6 mM, 7 mM, 8 mM, 9 mM, or 10 mM and LY2090314, and is administered to a subject, for example to the middle ear at a concentration of about 1 μM, 5 μM, 10 μM, 15 μM, 20 μM, or 40 μM and VPA is administered to a subject, for example to the middle ear at a concentration about 100 mM to 4,000 mM.

In some embodiments the EZH2 inhibitor is PF-06726304 and the Wnt agonist is a substituted 3-Imidazo[1,2-a]pyridin-3-yl-4-(1,2,3,4-tetrahydro-[1,4]diazepino-[6,7,1-hi]indol-7-yl)pyrrole-2,5-dione and the second epigenetic agent is VPA. In some embodiments, PF-06726304 is administered, in amount sufficient to achieve a concentration of about 0.1 μM, 0.2 μM, 0.3 μM, 0.4 uM, 0.5 μM, 0.6 μM, 0.7 μM, 0.8 μM, 0.9 μM, 1 μM, 2 μM, 3 μM, 4 μM, 5 μM, 6 μM, 7 μM, 8 μM, 9 μM, or 10 μM in the perilymph fluid in the inner ear and the substituted 3-Imidazo[1,2-a]pyridin-3-yl-4-(1,2,3,4-tetrahydro-[1,4]diazepino-[6,7,1-hi]indol-7-yl)pyrrole-2,5-dione is administered, in amount sufficient to achieve a concentration of about 1 nM, 5 nM, 10 nM, 15 nM, 20 nM, 50 nM, 100 nM, 250 nM, or 500 nM, in the perilymph fluid in the inner ear and VPA is administered in amount sufficient to achieve a concentration of about is about 100 μM to 4 mM in the perilymph fluid in the inner ear. Alternatively, PF-06726304 is administered to a subject, for example to the middle ear at a concentration of about 0.1 mM, 0.2 mM, 0.3 mM, 0.4 mM, 0.5 mM, 0.6 mM, 0.7 mM, 0.8 mM, 0.9 mM, 1 mM, 2 mM, 3 mM, 4 mM, 5 mM, 6 mM, 7 mM, 8 mM, 9 mM, or 10 mM and the substituted 3-Imidazo[1,2-a]pyridin-3-yl-4-(1,2,3,4-tetrahydro-[1,4]diazepino-[6,7,1-hi]indol-7-yl)pyrrole-5-dione and is administered to a subject, for example to the middle ear at a concentration of about 1 μM, 5 μM, 10 μM, 15 μM, 20 μM, 50 μM, 100 μM, 250 μM or 500 μM and VPA is administered to a subject, for example to the middle ear at a concentration about 100 mM to 4,000 mM.

In some embodiments the EZH2 inhibitor is PF-06726304 and the Wnt agonist is GSK3 inhibitor XXII and the second epigenetic agent is VPA. In some embodiments, PF-06726304 is administered, in amount sufficient to achieve a concentration of about 0.1 μM, 0.2 μM, 0.3 μM, 0.4 uM, 0.5 μM, 0.6 μM, 0.7 μM, 0.8 μM, 0.9 μM, 1 μM, 2 μM, 3 μM, 4 μM, 5 μM, 6 μM, 7 μM, 8 μM, 9 μM, or 10 μM in the perilymph fluid in the inner ear and GSK3-inhibitor XXII is administered, in amount sufficient to achieve a concentration of about 0.1 μM, 0.2 μM, 0.3 μM, 0.4 μM, 0.5 μM, 0.6 μM, 0.7 μM, 0.8 μM, 0.9 μM, or 1.0 μM, in the perilymph fluid in the inner ear and VPA is administered in amount sufficient to achieve a concentration of about is about 100 μM to 4 mM in the perilymph fluid in the inner ear. Alternatively, PF-06726304 is administered to a subject, for example to the middle ear at a concentration of about 0.1 mM, 0.2 mM, 0.3 mM, 0.4 mM, 0.5 mM, 0.6 mM, 0.7 mM, 0.8 mM, 0.9 mM, 1 mM, 2 mM, 3 mM, 4 mM, 5 mM, 6 mM, 7 mM, 8 mM, 9 mM, or 10 mM and the GSK3-inhibitor XXII is administered to a subject, for example to the middle ear at a concentration of about 0.1 mM, 0.2 mM, 0.3 mM, 0.4 mM, 0.5 mM, 0.6 mM, 0.7 mM, 0.8 mM, 0.9 mM, or 1.0 mM and VPA is administered to a subject, for example to the middle ear at a concentration about 100 mM to 4,000 mM.

In some embodiments the EZH2 inhibitor is PF-06726304 and the Wnt agonist is CHIR99021 and the second epigenetic agent is VPA. In some embodiments, PF-06726304 is administered, in amount sufficient to achieve a concentration of about 0.1 μM, 0.2 μM, 0.3 μM, 0.4 μM, 0.5 μM, 0.6 μM, 0.7 μM, 0.8 μM, 0.9 μM, 1 μM, 2 μM, 3 μM, 4 μM, 5 μM, 6 μM, 7 μM, 8 μM, 9 μM, or 10 μM in the perilymph fluid in the inner ear and CHIR99021 is administered, in amount sufficient to achieve a concentration of about 1 μM, 2 μM, 3 μM, 4 μM, 5 μM, 6 μM, 7 μM, 8 μM, 9 μM, or 10 μM, in the perilymph fluid in the inner ear and VPA is administered in amount sufficient to achieve a concentration of about is about 100 μM to 4 mM in the perilymph fluid in the inner ear. Alternatively, PF-06726304 is administered to a subject, for example to the middle ear at a concentration of about 0.1 mM, 0.2 mM, 0.3 mM, 0.4 mM, 0.5 mM, 0.6 mM, 0.7 mM, 0.8 mM, 0.9 mM, 1 mM, 2 mM, 3 mM, 4 mM, 5 mM, 6 mM, 7 mM, 8 mM, 9 mM, or 10 mM and CHIR99021 is administered to a subject, for example to the middle ear at a concentration of about 1 mM, 2 mM, 3 mM, 4 mM, 5 mM, 6 mM, 7 mM, 8 mM, 9 mM, or 10 mM and VPA is administered to a subject, for example to the middle ear at a concentration about 100 mM to 4,000 mM.

In some embodiments the DOT1L inhibitor is EPZ004777 and the Wnt agonist is AZD1080. In some embodiments, the EPZ004777 is administered, in an amount sufficient to achieve a concentration of about 10 nM, 20 nM, 30 nM, 40 nM, 50 nM, 60 nM, 70 nM, 80 nM, 90 nM, 100 nM, 200 nM, 300 nM, 400 nM, 500 nM, 600 nM, 700 nM, 800 nM, 900 nM, 1 μM, 2 μM, 3 μM, 4 μM, 5 μM, 6 μM, 7 μM, 8 μM, 9 μM, 10 μM, 11 μM, 12 μM, 13 μM, 14 μM, 15 μM, 20 μM, 25 μM, 30 μM, 35 μM, 40 μM, 45 μM, or about 50 μM in the perilymph fluid in the inner ear and AZD1080 is administered, in an amount sufficient to achieve a concentration of about is about 1 μM, 2 μM, 3 μM, 4 μM, 5 μM, 6 μM, 7 μM, 8 μM, 9 μM, or 10 μM, in the perilymph fluid in the inner ear. Alternatively, the EPZ004777 is administered to a subject, for example to the middle ear at a concentration of about 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, 10 mM, 11 mM, 12 mM, 13 mM, 14 mM, 15 mM, 20 mM, 25 mM, 30 mM, 35 mM, 40 mM, 45 mM, or about 50 mM and AZD1080, and is administered to a subject, for example to the middle ear at a concentration of about 1 mM, 2 mM, 3 mM, 4 mM, 5 mM, 6 mM, 7 mM, 8 mM, 9 mM, or 10 mM,

In some embodiments the DOT1L inhibitor is EPZ004777 and the Wnt agonist is LY2090314. In some embodiments, the EPZ004777 is administered, in an amount sufficient to achieve a concentration of about 10 nM, 20 nM, 30 nM, 40 nM, 50 nM, 60 nM, 70 nM, 80 nM, 90 nM, 100 nM, 200 nM, 300 nM, 400 nM, 500 nM, 600 nM, 700 nM, 800 nM, 900 nM, 1 μM, 2 μM, 3 μM, 4 μM, 5 μM, 6 μM, 7 μM, 8 μM, 9 μM, 10 μM, 11 μM, 12 μM, 13 μM, 14 μM, 15 μM, 20 μM, 25 μM, 30 μM, 35 μM, 40 μM, 45 μM, or about 50 μM in the perilymph fluid in the inner ear and LY2090314 is administered, in an amount sufficient to achieve a concentration of about 1 nM, 5 nM, 10 nM, 15 nM, 20 nM, or 40 nM in the perilymph fluid in the inner ear. Alternatively, the EPZ004777 is administered to a subject, for example to the middle ear at a concentration of about 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, 10 mM, 11 mM, 12 mM, 13 mM, 14 mM, 15 mM, 20 mM, 25 mM, 30 mM, 35 mM, 40 mM, 45 mM, or about 50 mM and LY2090314 is administered to a subject, for example to the middle ear at a concentration of about 1 μM, 5 μM, 10 μM, 15 μM, 20 μM, or 40 μM.

In some embodiments the DOT1L inhibitor is EPZ004777 and the Wnt agonist is a substituted 3-Imidazo [1,2-a]pyridin-3-yl-4-(1,2,3,4-tetrahydro-[1,4]diazepino-[6,7,1-hi]indol-7-yl)pyrrole-2,5-dione. In some embodiments, the EPZ004777 is administered, in an amount sufficient to achieve a concentration of about 10 nM, 20 nM, 30 nM, 40 nM, 50 nM, 60 nM, 70 nM, 80 nM, 90 nM, 100 nM, 200 nM, 300 nM, 400 nM, 500 nM, 600 nM, 700 nM, 800 nM, 900 nM, 1 μM, 2 μM, 3 μM, 4 μM, 5 μM, 6 μM, 7 μM, 8 μM, 9 μM, 10 μM, 11 μM, 12 μM, 13 μM, 14 μM, 15 μM, 20 μM, 25 μM, 30 μM, 35 μM, 40 μM, 45 μM, or about 50 μM in the perilymph fluid in the inner ear and the substituted 3-Imidazo[1,2-a]pyridin-3-yl-4-(1,2,3,4-tetrahydro-[1,4]diazepino-[6,7,1-hi]indol-7-y)pyrrole-2,5-dione is administered, in an amount sufficient to achieve a concentration of about 1 nM, 5 nM, 10 nM, 15 nM, 20 nM, 50 nM, 100 nM, 250 nM, or 500 nM, in the perilymph fluid in the inner ear. Alternatively, the EPZ004777 is administered to a subject, for example to the middle ear at a concentration of about 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, 10 mM, 11 mM, 12 mM, 13 mM, 14 mM, 15 mM, 20 mM, 25 mM, 30 mM, 35 mM, 40 mM, 45 mM, or about 50 mM and the substituted 3-Imidazo[1,2-a]pyridin-3-yl-4-(1,2,3,4-tetrahydro-[1,4]diazepino-[6,7,1-hi]indol-7-yl)pyrrole-2,5-dione and LY2090314 is administered to a subject, for example, to the middle ear at a concentration of about 1 μM, 5 μM, 10 μM, 15 μM, 20 μM, 50 μM, 100 μM, 250 μM, or 500 μM.

In some embodiments the DOT1L inhibitor is EPZ004777 and the Wnt agonist is GSK3 inhibitor XXII. In some embodiments, the EPZ004777 is administered, in an amount sufficient to achieve a concentration of about 10 nM, 20 nM, 30 nM, 40 nM, 50 nM, 60 nM, 70 nM, 80 nM, 90 nM, 100 nM, 200 nM, 300 nM, 400 nM, 500 nM, 600 nM, 700 nM, 800 nM, 900 nM, 1 μM, 2 μM, 3 μM, 4 μM, 5 μM, 6 μM, 7 μM, 8 μM, 9 μM, 10 μM, 11 μM, 12 μM, 13 μM, 14 μM, 15 μM, 20 μM, 25 μM, 30 μM, 35 μM, 40 μM, 45 μM, or about 50 μM in the perilymph fluid in the inner ear and GSK3-inhibitor XXII is administered in an amount sufficient to achieve a concentration of about 0.1 μM, 0.2 μM, 0.3 μM, 0.4 μM, 0.5 μM, 0.6 μM, 0.7 μM, 0.8 μM, 0.9 μM, or 1.0 μM, in the perilymph fluid in the inner ear. Alternatively, the EPZ004777 is administered to a subject, for example to the middle ear at a concentration of about 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, 10 mM, 11 mM, 12 mM, 13 mM, 14 mM, 15 mM, 20 mM, 25 mM, 30 mM, 35 mM, 40 mM, 45 mM, or about 50 mM and the GSK3-inhibitor XXII is administered, in an amount sufficient to achieve a concentration of about 0.1 mM, 0.2 mM, 0.3 mM, 0.4 mM, 0.5 mM, 0.6 mM, 0.7 mM, 0.8 mM, 0.9 mM, or 1.0 mM, in the perilymph fluid in the inner ear.

In some embodiments the DOT1L inhibitor is EPZ004777 and the Wnt agonist is CHIR99021. In some embodiments, the EPZ004777 is administered, in an amount sufficient to achieve a concentration of about 10 nM, 20 nM, 30 nM, 40 nM, 50 nM, 60 nM, 70 nM, 80 nM, 90 nM, 100 nM, 200 nM, 300 nM, 400 nM, 500 nM, 600 nM, 700 nM, 800 nM, 900 nM, 1 μM, 2 μM, 3 μM, 4 μM, 5 μM, 6 μM, 7 μM, 8 μM, 9 μM, 10 μM, 11 μM, 12 μM, 13 μM, 14 μM, 15 μM, 20 μM, 25 μM, 30 μM, 35 μM, 40 μM, 45 μM, or about 50 μM in the perilymph fluid in the inner ear and CHIR99021 is administered in an amount sufficient to achieve a concentration of about 1 μM, 2 μM, 3 μM, 4 μM, 5 μM, 6 μM, 7 μM, 8 μM, 9 μM, or 10 μM, in the perilymph fluid in the inner ear Alternatively, the EPZ004777 is administered to a subject, for example, to the middle ear at a concentration of about 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, 10 mM, 11 mM, 12 mM, 13 mM, 14 mM, 15 mM, 20 mM, 25 mM, 30 mM, 35 mM, 40 mM, 45 mM, or about 50 mM and CHIR99021 is administered to a subject, for example to the middle ear at a concentration of about 1 mM, 2 mM, 3 mM, 4 mM, 5 mM, 6 mM, 7 mM, 8 mM, 9 mM, or 10 mM.

In some embodiments the DOT1L inhibitor is pinometostat and the Wnt agonist is AZD1080. In some embodiments, the pinometostat is administered, in an amount sufficient to achieve a concentration of about 10 nM, 20 nM, 30 nM, 40 nM, 50 nM, 60 nM, 70 nM, 80 nM, 90 nM, 100 nM, 200 nM, 300 nM, 400 nM, 500 nM, 600 nM, 700 nM, 800 nM, 900 nM, 1 μM, 2 μM, 3 μM, 4 μM, 5 μM, 6 μM, 7 μM, 8 μM, 9 μM, 10 μM, 11 μM, 12 μM, 13 μM, 14 μM, 15 μM, 20 μM, 25 μM, 30 μM, 35 μM, 40 μM, 45 μM, or about 50 μM in the perilymph fluid in the inner ear and AZD1080 is administered, in an amount sufficient to achieve a concentration of about is about 1 μM, 2 μM, 3 μM, 4 μM, 5 μM, 6 μM, 7 μM, 8 μM, 9 μM, or 10 μM, in the perilymph fluid in the inner ear. Alternatively, the pinometostat is administered to a subject, for example to the middle ear at a concentration of about 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, 10 mM, 11 mM, 12 mM, 13 mM, 14 mM, 15 mM, 20 mM, 25 mM, 30 mM, 35 mM, 40 mM, 45 mM, or about 50 mM and AZD1080, and is administered to a subject, for example to the middle ear at a concentration of about 1 mM, 2 mM, 3 mM, 4 mM, 5 mM, 6 mM, 7 mM, 8 mM, 9 mM, or 10 mM.

In some embodiments the DOT1L inhibitor is pinometostat and the Wnt agonist is LY2090314. In some embodiments, the pinometostat is administered, in an amount sufficient to achieve a concentration of about 10 nM, 20 nM, 30 nM, 40 nM, 50 nM, 60 nM, 70 nM, 80 nM, 90 nM, 100 nM, 200 nM, 300 nM, 400 nM, 500 nM, 600 nM, 700 nM, 800 nM, 900 nM, 1 μM, 2 μM, 3 μM, 4 μM, 5 μM, 6 μM, 7 μM, 8 μM, 9 μM, 10 μM, 11 μM, 12 μM, 13 μM, 14 μM, 15 μM, 20 μM, 25 μM, 30 μM, 35 μM, 40 μM, 45 μM, or about 50 μM in the perilymph fluid in the inner ear and LY2090314 is administered in an amount sufficient to achieve a concentration of about 1 nM, 5 nM, 10 nM, 15 nM, 20 nM or 40 nM, in the perilymph fluid in the inner ear. Alternatively, the pinometostat is administered to a subject, for example to the middle ear at a concentration of about 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, 10 mM, 11 mM, 12 mM, 13 mM, 14 mM, 15 mM, 20 mM, 25 mM, 30 mM, 35 mM, 40 mM, 45 mM, or about 50 mM and LY20903 is administered to a subject, for example, to the middle ear at a concentration of about 1 μM, 5 μM, 10 μM, 15 μM, 20 μM, or 40 μM.

In some embodiments the DOT1L inhibitor is pinometostat and the Wnt agonist is a substituted 3-Imidazo[1,2-a]pyridin-3-yl-4-(1,2,3,4-tetrahydro-[1,4]diazepino-[6,7,1-hi]indol-7-yl)pyrrole-2,5-dione. In some embodiments, the pinometostat is administered, in an amount sufficient to achieve a concentration of about 10 nM, 20 nM, 30 nM, 40 nM, 50 nM, 60 nM, 70 nM, 80 nM, 90 nM, 100 nM, 200 nM, 300 nM, 400 nM, 500 nM, 600 nM, 700 nM, 800 nM, 900 nM, 1 μM, 2 μM, 3 μM, 4 μM, 5 μM, 6 μM, 7 μM, 8 μM, 9 μM, 10 μM, 11 μM, 12 μM, 13 μM, 14 μM, 15 μM, 20 μM, 25 μM, 30 μM, 35 μM, 40 μM, 45 μM, or about 50 μM in the perilymph fluid in the inner ear and the substituted 3-Imidazo[1,2-a]pyridin-3-yl-4-(1,2,3,4-tetrahydro-[1,4]diazepino-[6,7,1-hi]indol-7-yl)pyrrole-2,5-dione is administered, in an amount sufficient to achieve a concentration of about 1 nM, 5 nM, 10 nM, 15 nM, 20 nM 50 nM, 100 nM, 250 nM, or 500 nM, in the perilymph fluid in the inner ear. Alternatively, the pinometostat is administered to a subject, for example to the middle ear at a concentration of about 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, 10 mM, 11 mM, 12 mM, 13 mM, 14 mM, 15 mM, 20 mM, 25 mM, 30 mM, 35 mM, 40 mM, 45 mM, or about 50 mM and the substituted 3-Imidazo[1,2-a]pyridin-3-yl-4-(1,2,3,4-tetrahydro-[1,4]diazepino-[6,7,1-hi]indol-7-yl)pyrrole-2,5-dione and is administered to a subject, for example, to the middle ear at a concentration of about 1 μM, 5 μM, 10 μM, 15 μM, 20 μM, 50 μM, 100 μM, 250 μM, or 500 μM.

In some embodiments the DOT1L inhibitor is pinometostat and the Wnt agonist is GSK3 inhibitor XXII. In some embodiments, the pinometostat is administered, in an amount sufficient to achieve a concentration of about 10 nM, 20 nM, 30 nM, 40 nM, 50 nM, 60 nM, 70 nM, 80 nM, 90 nM, 100 nM, 200 nM, 300 nM, 400 nM, 500 nM, 600 nM, 700 nM, 800 nM, 900 nM, 1 μM, 2 μM, 3 μM, 4 μM, 5 μM, 6 μM, 7 μM, 8 μM, 9 μM, 10 μM, 11 μM, 12 μM, 13 μM, 14 μM, 15 μM, 20 μM, 25 μM, 30 μM, 35 μM, 40 μM, 45 μM, or about 50 μM in the perilymph fluid in the inner ear and GSK3-inhibitor XXII is administered, in an amount sufficient to achieve a concentration of about 0.1 μM, 0.2 μM, 0.3 μM, 0.4 μM, 0.5 μM, 0.6 μM, 0.7 μM, 0.8 μM, 0.9 μM, or 1.0 μM, in the perilymph fluid in the inner ear. Alternatively, the pinometostat is administered to a subject, for example to the middle ear at a concentration of about 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, 10 mM, 11 mM, 12 mM, 13 mM, 14 mM, 15 mM, 20 mM, 25 mM, 30 mM, 35 mM, 40 mM, 45 mM, or about 50 mM and the GSK3-inhibitor XXII is administered, in an amount sufficient to achieve a concentration of about 0.1 mM, 0.2 mM, 0.3 mM, 0.4 mM, 0.5 mM, 0.6 mM, 0.7 mM, 0.8 mM, 0.9 mM, or 1.0 mM, in the perilymph fluid in the inner ear.

In some embodiments the DOT1L inhibitor is pinometostat and the Wnt agonist is CHIR99021. In some embodiments, the pinometostat is administered, in an amount sufficient to achieve a concentration of about 10 nM, 20 nM, 30 nM, 40 nM, 50 nM, 60 nM, 70 nM, 80 nM, 90 nM, 100 nM, 200 nM, 300 nM, 400 nM, 500 nM, 600 nM, 700 nM, 800 nM, 900 nM, 1 μM, 2 μM, 3 μM, 4 μM, 5 μM, 6 μM, 7 μM, 8 μM, 9 μM, 10 μM, 11 μM, 12 μM, 13 μM, 14 μM, 15 μM, 20 μM, 25 μM, 30 μM, 35 μM, 40 μM, 45 μM, or about 50 μM in the perilymph fluid in the inner ear and CHIR99021 is administered, in an amount sufficient to achieve a concentration of about 1 μM, 2 μM, 3 μM, 4 μM, 5 μM, 6 μM, 7 μM, 8 μM, 9 μM, or 10 μM, in the perilymph fluid in the inner ear Alternatively, the pinometostat is administered to a subject, for example, to the middle ear at a concentration of about 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, 10 mM, 11 mM, 12 mM, 13 mM, 14 mM, 15 mM, 20 mM, 25 mM, 30 mM, 35 mM, 40 mM, 45 mM, or about 50 mM and CHIR99021 is administered to a subject, for example to the middle ear at a concentration of about 1 mM, 2 mM, 3 mM, 4 mM, 5 mM, 6 mM, 7 mM, 8 mM, 9 mM, or 10 mM.

In some embodiments the DOT1L inhibitor is SGC0946 and the Wnt agonist is AZD1080. In some embodiments, the SGC0946 is administered, in an amount sufficient to achieve a concentration of about 10 nM, 20 nM, 30 nM, 40 nM, 50 nM, 60 nM, 70 nM, 80 nM, 90 nM, 100 nM, 200 nM, 300 nM, 400 nM, 500 nM, 600 nM, 700 nM, 800 nM, 900 nM, 1 μM, 2 μM, 3 μM, 4 μM, 5 μM, 6 μM, 7 μM, 8 μM, 9 μM, 10 μM, 11 μM, 12 μM, 13 μM, 14 μM, 15 μM, 20 μM, 25 μM, 30 μM, 35 μM, 40 μM, 45 μM, or about 50 μM in the perilymph fluid in the inner ear. Alternatively, the SGC0946 is administered to a subject, for example, to the middle ear at a concentration of about 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, 10 mM, 11 mM, 12 mM, 13 mM, 14 mM, 15 mM, 20 mM, 25 mM, 30 mM, 35 mM, 40 mM, 45 mM, or about 50 mM and AZD1080 is administered to a subject, for example, to the middle ear at a concentration of about 1 mM, 2 mM, 3 mM, 4 mM, 5 mM, 6 mM, 7 mM, 8 mM, 9 mM, or 10 mM.

In some embodiments the DOT1L inhibitor is SGC0946 and the Wnt monist is LY209031. In some embodiments, the SGC0946 is administered, in an amount sufficient to achieve a concentration of about 10 nM, 20 nM, 30 nM, 40 nM, 50 nM, 60 nM, 70 nM, 80 mM, 90 nM, 100 nM, 200 nM, 300 nM, 400 nM, 500 nM, 600 nM, 700 nM, 800 nM, 900 nM, 1 μM, 2 μM, 3 μM, 4 μM, 5 μM, 6 μM, 7 μM, 8 μM, 9 μM, 10 μM, 11 μM, 12 μM, 13 μM, 14 μM, 15 μM, 20 μM, 25 μM, 30 μM, 35 μM, 40 μM, 45 μM, or about 50 μM in the perilymph fluid in the inner ear. Alternatively, the SGC0946 is administered to a subject, for example, to the middle ear at a concentration of about 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, 10 mM, 11 mM, 12 mM, 13 mM, 14 mM, 15 mM, 20 mM, 25 mM, 30 mM, 35 mM, 40 mM, 45 mM, or about 50 mM and LY2090314 is administered to a subject, for example, to the middle ear at a concentration of about 1 μM, 5 μM, 10 μM, 15 μM, 20 μM, or 40 nM.

In some embodiments the DOT1L inhibitor is SGC0946 and the Wnt agonist is a substituted 3-Imidazo[1,2-a]pyridin-3-yl-4-(1,2,3,4-tetrahydro-[1,4]diazepino-[6,7,1-hi]indol-7-yl)pyrrole-2,5-dione. In some embodiments, the SGC0946 is administered, in an amount sufficient to achieve a concentration of about 10 nM, 20 nM, 30 nM, 40 nM, 50 nM, 60 nM, 70 nM, 80 nM, 90 nM, 100 nM, 200 nM, 300 nM, 400 nM, 500 nM, 600 nM, 700 nM, 800 nM, 900 nM, 1 μM, 2 μM, 3 μM, 4 μM, 5 μM, 6 μM, 7 μM, 8 μM, 9 μM, 10 μM, 11 μM, 12 μM, 13 μM, 14 μM, 15 μM, 20 μM, 25 μM, 30 μM, 35 μM, 40 μM, 45 μM, or about 50 μM in the perilymph fluid in the inner ear and the substituted 3-Imidazo[1,2-a]pyridin-3-yl-4-(1,2,3,4-tetrahydro-[1,4]diazepino-[6,7,1-hi]indol-7-yl)pyrrole-2,5-dione is administered, in an amount sufficient to achieve a concentration of about 1 nM, 5 nM, 10 nM, 15 nM, 20 nM, 50 nM, 100 nM, 250 nM, or 500 nM, in the perilymph fluid in the inner ear. Alternatively, the SGC0946 is administered to a subject, for example, to the middle ear at a concentration of about 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, 10 mM, 11 mM, 12 mM, 13 mM, 14 mM, 15 mM, 20 mM, 25 mM, 30 mM, 35 mM, 40 mM, 45 mM, or about 50 mM and the substituted 3-Imidazo[1,2-a]pyridin-3-yl-4-(1,2,3,4-tetrahydro-[1,4]diazepino-[6,7,1-hi]indol-7-yl)pyrrole-2,5-dione and is administered to a subject, for example, to the middle ear at a concentration of about 1 μM, 5 μM, 10 μM, 15 μM, 20 μM, 50 μM, 100 μM, 250 μM, 500 μM.

In some embodiments the DOT1L inhibitor is SGC0946 and the Wnt agonist is GSK3 inhibitor XXII. In some embodiments, the SGC0946 is administered, in an amount sufficient to achieve a concentration of about 10 nM, 20 nM, 30 nM, 40 nM, 50 nM, 60 nM, 70 nM, 80 nM, 90 nM, 100 nM, 200 nM, 300 nM, 400 nM, 500 nM, 600 nM, 700 nM, 800 nM, 900 nM, 1 μM, 2 μM, 3 μM, 4 μM, 5 μM, 6 μM, 7 μM, 8 μM, 9 μM, 10 μM, 11 μM, 13 μM, 14 μM, 15 μM, 20 μM, 25 μM, 30 μM, 35 μM, 40 μM, 45 μM, or about 50 μM in the perilymph fluid in the inner ear and GSK3-inhibitor XXII is administered in an amount sufficient to achieve a concentration of about 0.1 μM, 0.2 μM, 0.3 μM, 0.4 μM, 0.5 μM, 0.6 μM, 0.7 μM, 0.8 μM, 0.9 μM, or 1.0 μM, in the perilymph fluid in the inner ear. Alternatively, the SGC0946 is administered to a subject, for example, to the middle ear at a concentration of about 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, 10 mM, 11 mM, 12 mM, 13 mM, 14 mM, 15 mM, 20 mM, 25 mM, 30 mM, 35 mM, 40 mM, 45 mM, or about 50 mM and the GSK3-inhibitor XXII is administered, in an amount sufficient to achieve a concentration of about 0.1 mM, 0.2 mM, 0.3 mM, 0.4 mM, 0.5 mM, 0.6 mM, 0.7 mM, 0.8 mM, 0.9 mM, or 1.0 mM, in the perilymph fluid in the inner ear.

In some embodiments the DOT1L inhibitor is SGC0946 and the Wnt agonist is CHIR99021. In some embodiments, the SGC0946 is administered, in an amount sufficient to achieve a concentration of about 10 nM, 20 nM, 30 nM, 40 nM, 50 nM, 60 nM, 70 nM, 80 nM, 90 nM, 100 nM, 200 nM, 300 nM, 400 nM, 500 nM, 600 nM, 700 nM, 800 nM, 900 nM, 1 μM, 2 μM, 3 μM, 4 μM, 5 μM, 6 μM, 7 μM, 8 μM, 9 μM, 10 μM, 11 μM, 12 μM, 13 μM, 14 μM, 15 μM, 20 μM, 25 μM, 30 μM, 35 μM, 40 μM, 45 μM, or about 50 μM in the perilymph fluid in the inner ear. Alternatively, the SGC0946 is administered to a subject, for example, to the middle ear at a concentration of about 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, 10 mM, 11 mM, 12 mM, 13 mM, 14 mM, 15 mM, 20 mM, 25 mM, 30 mM, 35 mM, 40 mM, 45 mM, or about 50 mM and CHIR99021 is administered to a subject, for example to the middle ear at a concentration of about 1 mM, 2 mM, 3 mM, 4 mM, 5 mM, 6 mM, 7 mM, 8 mM, 9 mM, or 10 mM.

In some embodiments the DOT1L inhibitor is EPZ004777, the Wnt agonist is AZD1080 and the second epigenetic agent is VPA. In some embodiments, the EPZ004777 is administered, in an amount sufficient to achieve a concentration of about 10 nM, 20 nM, 30 nM, 40 nM, 50 nM, 60 nM, 70 nM, 80 nM, 90 nM, 100 nM, 200 nM, 300 nM, 400 nM, 500 nM, 600 nM, 700 nM, 800 nM, 900 nM, 1 μM, 2 μM, 3 μM, 4 μM, 5 μM, 6 μM, 7 μM, 8 μM, 9 μM, 10 μM, 11 μM, 12 μM, 13 μM, 14 μM, 15 μM, 20 μM, 25 μM, 30 μM, 35 μM, 40 μM, 45 μM, or about 50 μM in the perilymph fluid in the inner ear; AZD1080 is administered, in an amount sufficient to achieve a concentration of about is about 1 μM, 2 μM, 3 μM, 4 μM, 5 μM, 6 μM, 7 μM, 8 μM, 9 μM, or 10 μM, in the perilymph fluid in the inner ear and VPA is administered in an amount sufficient to achieve a concentration of about is about 100 μM to 4 mM in the perilymph fluid in the inner ear. Alternatively, the EPZ004777 is administered to a subject, for example, to the middle ear at a concentration of about 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, 10 mM, 11 mM, 12 mM, 13 mM, 14 mM, 15 mM, 20 mM, 25 mM, 30 mM, 35 mM, 40 mM, 45 mM, or about 50 mM; AZD1080 is administered to a subject, for example to the middle ear at a concentration of about 1 mM, 2 mM, 3 mM, 4 mM, 5 mM, 6 mM, 7 mM, 8 mM, 9 mM, or 10 mM and VPA to a subject, for example, to the middle ear at a concentration about 100 mM to 4,000 mM.

In some embodiments the DOT1L inhibitor is EPZ004777, the Wnt agonist is LY2090314 and the second epigenetic agent is VPA. In some embodiments, the EPZ004777 is administered, in an amount sufficient to achieve a concentration of about 10 nM, 20 nM, 30 nM, 40 nM, 50 nM, 60 nM, 70 nM, 80 nM, 90 nM, 100 nM, 200 nM, 300 nM, 400 nM, 500 nM, 600 nM, 700 nM, 800 nM, 900 nM, 1 μM, 2 μM, 3 μM, 4 μM, 5 μM, 6 μM, 7 μM, 8 μM, 9 μM, 10 μM, 11 μM, 12 μM, 13 μM, 14 μM, 15 μM, 20 μM, 25 μM, 30 μM, 35 μM, 40 μM, 45 μM, or about 50 μM in the perilymph fluid in the inner ear; LY2090314 is administered, in an amount sufficient to achieve a concentration of about 1 nM, 5 nM, 10 nM, 15 nM, 20 nM or 40 nM in the perilymph fluid in the inner ear and VPA is administered in an amount sufficient to achieve a concentration of about is about 100 μM to 4 mM in the perilymph fluid in the inner ear. Alternatively, the EPZ004777 is administered to a subject, for example to the middle ear at a concentration of about 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, 10 mM, 11 mM, 12 mM, 13 mM, 14 mM, 15 mM, 20 mM, 25 mM, 30 mM, 35 mM, 40 mM, 45 mM, or about 50 mM and the LY2090314 is administered to a subject, for example to the middle ear at a concentration of about 1 μM, 5 μM, 10 μM, 15 μM, 20 μM, or 40 μM and VPA is administers to a subject, for example, to the middle ear at a concentration about 100 mM to 4,000 mM.

In some embodiments the DOT1L inhibitor is EPZ004777 and the Wnt agonist is a substituted 3-Imidazo[1,2-a]pyridin-3-yl-4-(1,2,3,4-tetrahydro-[1,4]diazepino-[6,7,1-hi]indol-7-yl)pyrrole-2,5-dione and the second epigenetic agent is VPA. In some embodiments, the EPZ004777 is administered, in an amount sufficient to achieve a concentration of about 10 nM, 20 nM, 30 nM, 40 nM, 50 nM, 60 nM, 70 nM, 80 nM, 90 nM, 100 nM, 200 nM, 300 nM, 400 nM, 500 nM, 600 nM, 700 nM, 800 nM, 900 nM, 1 μM, 2 μM, 3 μM, 4 μM, 5 μM, 6 μM, 7 μM, 8 μM, 9 μM, 10 μM, 11 μM, 12 μM, 13 μM, 14 μM, 15 μM, 20 μM, 25 μM, 30 μM, 35 μM, 40 μM, 45 μM, or about 50 μM in the perilymph fluid in the inner ear; the substituted 3-Imidazo[1,2-a]pyridin-3-yl-4-(1,2,3,4-tetrahydro-[1,4]diazepino-[6,7,1-hi]indol-7-yl)pyrrole-2,5-dione is administered, in an amount sufficient to achieve a concentration of about 1 nM, 5 nM, 10 nM, 15 nM, 20 nM, 50 nM, 100 nM, 250 nM, or 500 nM, in the perilymph fluid in the inner ear and VPA is administered in an amount sufficient to achieve a concentration of about is about 100 μM to 4 μM in the perilymph fluid in the inner ear. Alternatively, the EPZ004777 is administered to a subject, for example, to the middle ear at a concentration of about 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, 10 mM, 11 mM, 12 mM, 13 mM, 14 mM, 15 mM, 20 mM, 25 mM, 30 mM, 35 mM, 40 mM, 45 mM, or about 50 mM and the substituted 3-Imidazo[1, 2-a]pyridin-3-yl-4-(1,2,3,4-tetrahydro-[1,4]diazepino-[6,7,1-hi]indol-7-yl)pyrrole-2,5-dione and is administered to a subject, for example to the middle ear at a concentration of about 1 μM, 5 μM, 10 μM, 15 μM, 20 μM, 50 μM, 100 μM, 250 μM, or 500 μM and VPA is administers to a subject, for example to the middle ear at a concentration about 100 mM to 4,000 mM.

In some embodiments the DOT1L inhibitor is EPZ004777 and the Wnt agonist is GSK3 inhibitor XXII and the second epigenetic agent is VPA. In some embodiments, the EPZ004777 is administered, in an amount sufficient to achieve a concentration of about 10 nM, 20 nM, 30 nM, 40 nM, 50 nM, 60 nM, 70 nM, 80 nM, 90 nM, 100 nM, 200 nM, 300 nM, 400 nM, 500 nM, 600 nM, 700 nM, 800 nM, 900 nM, 1 μM, 2 μM, 3 μM, 4 μM, 5 μM, 6 μM, 7 μM, 8 μM, 9 μM, 10 μM, 11 μM, 12 μM, 13 μM, 14 μM, 15 μM, 20 μM, 25 μM, 30 μM, 35 μM, 40 μM, 45 μM, or about 50 μM in the perilymph fluid in the inner ear; the GSK3-inhibitor XXII is administered, in an amount sufficient to achieve a concentration of about 0.1 μM, 0.2 μM, 0.3 μM, 0.4 μM, 0.5 μM, 0.6 μM, 0.7 μM, 0.8 μM, 0.9 μM, or 1.0 μM, in the perilymph fluid in the inner ear and VPA is administered in an amount sufficient to achieve a concentration of about is about 10 100 μM to 4 mM in the perilymph fluid in the inner ear. Alternatively, the EPZ004777 is administered to a subject, for example to the middle ear at a concentration of about 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, 10 mM, 11 mM, 12 mM, 13 mM, 14 mM, 15 mM, 20 mM, 25 mM, 30 mM, 35 mM, 40 mM, 45 mM, or about 50 mM and the GSK3-inhibitor XXII is administered, in an amount sufficient to achieve a concentration of about 0.1 mM, 0.2 mM, 0.3 mM, 0.4 mM, 0.5 mM, 0.6 mM, 0.7 mM, 0.8 mM, 0.9 mM, or 1.0 mM, in the perilymph fluid in the inner ear and VPA is administers to a subject, for example, to the middle ear at a concentration about 100 mM to 4,000 mM.

In some embodiments the DOT1L inhibitor is EPZ004777 and the Wnt agonist is CHIR99021 and the second epigenetic agent is VPA. In some embodiments, the EPZ004777 is administered, in an amount sufficient to achieve a concentration of about 10 nM, 20 nM, 30 nM, 40 nM, 50 nM, 60 nM, 70 nM, 80 nM, 90 nM, 100 nM, 200 nM, 300 nM, 400 nM, 500 nM, 600 nM, 700 nM, 800 nM, 900 nM, 1 μM, 2 μM, 3 μM, 4 μM, 5 μM, 6 μM, 7 μM, 8 μM, 9 μM, 10 μM, 11 μM, 12 μM, 13 μM, 14 μM, 15 μM, 20 μM, 25 μM, 30 μM, 35 μM, 40 μM, 45 μM, or about 50 μM in the perilymph fluid in the inner ear; CHIR99021 is administered, in an amount sufficient to achieve a concentration of about 1 μM, 2 μM, 3 μM, 4 μM, 5 μM, 6 μM, 7 μM, 8 μM, 9 μM, or 10 μM, in the perilymph fluid in the inner ear and VPA is administered in an amount sufficient to achieve a concentration of about is about 100 μM to 4 mM in the perilymph fluid in the inner ear. Alternatively, the EPZ004777 is administered to a subject, for example, to the middle ear at a concentration of about 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, 10 mM, 11 mM, 12 mM, 13 mM, 14 mM, 15 mM, 20 mM, 25 mM, 30 mM, 35 mM, 40 mM, 45 mM, or about 50 mM and CHIR99021 is administered to a subject, for example to the middle ear at a concentration of about 1 mM, 2 mM, 3 mM, 4 mM, 5 mM, 6 mM, 7 mM, 8 mM, 9 mM, or 10 mM and VPA is administered to a subject, for example, to the middle ear at a concentration about 100 mM to 4,000 mM.

In some embodiments the DOT1L inhibitor is pinometostat and the Wnt agonist is AZD1080 and the second epigenetic agent is VPA. In some embodiments, the pinometostat is administered, in an amount sufficient to achieve a concentration of about 10 nM, 20 nM, 30 nM, 40 nM, 50 nM, 60 nM, 70 nM, 80 nM, 90 nM, 100 nM, 200 nM, 300 nM, 400 nM, 500 nM, 600 nM, 700 nM, 800 nM, 900 nM, 1 μM, 2 μM, 3 μM, 4 μM, 5 μM, 6 μM, 7 μM, 8 μM, 9 μM, 10 μM, 11 μM, 12 μM, 13 μM, 14 μM, 15 μM, 20 μM, 25 μM, 30 μM, 35 μM, 40 μM, 45 μM, or about 50 μM in the perilymph fluid in the inner ear; AZD1080 is administered, in an amount sufficient to achieve a concentration of about is about 1 μM, 2 μM, 3 μM, 4 μM, 5 μM, 6 μM, 6 μM, 7 μM, 8 μM, 9 μM, or 10 μM, in the perilymph fluid in the inner ear and VPA is administered in an amount sufficient to achieve a concentration of about is about 100 μM to 4 mM in the perilymph fluid in the inner ear. Alternatively, the pinometostat is administered to a subject, for example, to the middle ear at a concentration of about 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, 10 mM, 11 mM, 12 mM, 13 mM, 14 mM, 15 mM, 20 mM, 25 mM, 30 mM, 35 mM, 40 mM, 45 mM, or about 50 mM and AZD1080, and is administered to a subject, for example, to the middle ear at a concentration of about 1 mM, 2 mM, 3 mM, 4 mM, 5 mM, 6 mM, 7 mM, 8 mM, 9 mM, or 10 mM and VPA is administered to a subject, for example, to the middle ear at a concentration about 100 mM to 4,000 mM.

In some embodiments the DOT1L inhibitor is pinometostat and the Wnt agonist is LY2090314 and the second epigenetic agent is VPA. In some embodiments, the pinometostat is administered, in an amount sufficient to achieve a concentration of about 10 nM, 20 nM, 30 nM, 40 nM, 50 nM, 60 nM, 70 nM, 80 nM, 90 nM, 100 nM, 200 nM, 300 nM, 400 nM, 500 nM, 600 nM, 700 nM, 800 nM, 900 nM, 1 μM, 2 μM, 3 μM, 4 μM, 5 μM, 6 μM, 7 μM, 8 μM, 9 μM, 10 μM, 11 μM, 12 μM, 13 μM, 14 μM, 15 μM, 20 μM, 25 μM, 30 μM, 35 μM, 40 μM, 45 μM, or about 50 μM in the perilymph fluid in the inner ear; LY2090314 is administered, in an amount sufficient to achieve a concentration of about 1 nM, 5 nM, 10 nM, 15 nM, 20 nM or 40 nM in the perilymph fluid in the inner ear and VPA is administered in an amount sufficient to achieve a concentration of about is about 100 μM to 4 mM in the perilymph fluid in the inner ear. Alternatively, the pinometostat is administered to a subject, for example, to the middle ear at a concentration of about 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, 10 mM, 11 mM, 12 mM, 13 mM, 14 mM, 15 mM, 20 mM, 25 mM, 30 mM, 35 mM, 40 mM, 45 mM, or about 50 mM and LY2090314 is administered to a subject, for example to the middle ear at a concentration of about 1 μM, 5 μM, 10 μM, 15 μM, 20 μM or 40 μM and VPA is administers to a subject, for example, to the middle ear at a concentration about 100 mM to 4,000 mM.

In some embodiments the DOT1L inhibitor is pinometostat and the Wnt agonist is a substituted 3-Imidazo[1,2-a]pyridin-3-yl-4-(1,2,3,4-tetrahydro-[1,4]diazepino-[6,7,1-hi]indol-7-yl)pyrrole-2,5-dione and the second epigenetic agent is VPA. In some embodiments, the pinometostat is administered, in an amount sufficient to achieve a concentration of about 10 nM, 20 nM, 30 nM, 40 nM, 50 nM, 60 nM, 70 nM, 80 nM, 90 nM, 100 nM, 200 nM, 300 nM, 400 nM, 500 nM, 600 nM, 700 nM, 800 nM, 900 nM, 1 μM, 2 μM, 3 μM, 4 μM, 5 μM, 6 μM, 7 μM, 8 μM, 9 μM, 10 μM, 11 μM, 12 μM, 13 μM, 14 μM, 15 μM, 20 μM, 25 μM, 30 μM, 35 μM, 40 μM, 45 μM, or about 50 μM in the perilymph fluid in the inner ear; the substituted 3-Imidazo[1,2-a]pyridin-3-yl-4-(1,2,3,4-tetrahydro-[1,4]diazepino-[6,7,1-hi]indol-7-yl)pyrrole-2,5-dione is administered, in an amount sufficient to achieve a concentration of about 1 nM, 5 nM, 10 nM, 15 nM, 20 nM, 50 nM, 100 nM, 250 nM, or 500 nM, in the perilymph fluid in the inner ear and VPA is administered in an amount sufficient to achieve a concentration of about is about 100 μM to 4 mM in the perilymph fluid in the inner ear. Alternatively, the pinometostat is administered to a subject, for example to the middle ear at a concentration of about 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, 10 mM, 11 mM, 12 mM, 13 mM, 14 mM, 15 mM, 20 mM, 25 mM, 30 mM, 35 mM, 40 mM, 45 mM, or about 50 mM and the substituted 3-Imidazo[1,2-a]pyridin-3-yl-4-(1,2,3,4-tetrahydro-[1,4]diazepino-[6,7,1-hi]indol-7-yl)pyrrole-2,5-dione and is administered to a subject, for example to the middle ear at a concentration of about 1 μM, 5 μM, 10 μM, 15 μM, 20 μM, 50 μM, 100 μM, 250 μM, or 500 μM and VPA is administers to a subject, for example to the middle ear at a concentration about 100 mM to 4,000 mM.

In some embodiments the DOT1L inhibitor is pinometostat and the Wnt agonist is GSK3 inhibitor XXII and the second epigenetic agent is VPA. In some embodiments, the pinometostat is administered, in an amount sufficient to achieve a concentration of about 10 nM, 20 nM, 30 nM, 40 nM, 50 nM, 60 nM, 70 nM, 80 nM, 90 nM, 100 nM, 200 nM, 300 nM, 400 nM, 500 nM, 600 nM, 700 nM, 800 nM, 900 nM, 1 μM, 2 μM, 3 μM, 4 μM, 5 μM, 6 μM, 7 μM, 8 μM, 9 μM, 10 μM, 11 μM, 12 μM, 13 μM, 14 μM, 15 μM, 20 μM, 25 μM, 30 μM, 35 μM, 40 μM, 45 μM, or about 50 μM in the perilymph fluid in the inner ear; GSK3-inhibitor XXII is administered, in an amount sufficient to achieve a concentration of about 0.1 μM, 0.2 μM, 0.3 μM, 0.4 μM, 0.5 μM, 0.6 μM, 0.7 μM, 0.8 μM, 0.9 μM, or 1.0 μM, in the perilymph fluid in the inner ear and VPA is administered in an amount sufficient to achieve a concentration of about is about 100 μM to 4 mM in the perilymph fluid in the inner ear. Alternatively, the pinometostat is administered to a subject, for example to the middle ear at a concentration of about 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, 10 mM, 11 mM, 12 mM, 13 mM, 14 mM, 15 mM, 20 mM, 25 mM, 30 mM, 35 mM, 40 mM, 45 mM, or about 50 mM and the GSK3-inhibitor XXII is administered to a subject, for example to the middle ear at a concentration about 0.1 mM, 0.2. mM, 0.3 mM, 0.4 mM, 0.5 mM, 0.6 mM, 0.7 mM, 0.8 mM, 0.9 mM, or 1.0 mM and VPA is administered to a subject, for example, to the middle ear at a concentration about 100 mM to 4,000 mM.

In some embodiments the DOT1L inhibitor is pinometostat and the Wnt agonist is CHIR99021 and the second epigenetic agent is VPA. In some embodiments, the pinometostat is administered, in an amount sufficient to achieve a concentration of about 10 nM, 20 nM, 30 nM, 40 nM, 50 nM, 60 nM, 70 nM, 80 nM, 90 nM, 100 nM, 200 nM, 300 nM, 400 nM, 500 nM, 600 nM, 700 nM, 800 nM, 900 nM, 1 μM, 2 μM, 3 μM, 4 μM, 5 μM, 6 μM, 7 μM, 8 μM, 9 μM, 10 μM, 11 μM, 12 μM, 13 μM, 14 μM, 15 μM, 20 μM, 25 μM, 30 μM, 35 μM, 40 μM, 45 μM, or about 50 μM in the perilymph fluid in the inner ear; CHIR99021 is administered, in an amount sufficient to achieve a concentration of about 1 μM, 2 μM, 3 μM, 4 μM, 5 μM, 6 μM, 7 μM, 8 μM, 9 μM, or 10 μM, in the perilymph fluid in the inner ear and VPA is administered in an amount sufficient to achieve a concentration of about is about 100 μM to 4 mM in the perilymph fluid in the inner ear. Alternatively, the pinometostat is administered to a subject, for example, to the middle ear at a concentration of about 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, 10 mM, 11 mM, 12 mM, 13 mM, 14 mM, 15 mM, 20 mM, 25 mM, 30 mM, 35 mM, 40 mM, 45 mM, or about 50 mM and CHIR99021 is administered to a subject, for example to the middle ear at a concentration of about 1 mM, 2 mM, 3 mM, 4 mM, 5 mM, 6 mM, 7 mM, 8 mM, 9 mM, or 10 mM and VPA is administered to a subject, for example, to the middle ear at a concentration about 100 mM to 4,000 mM.

In some embodiments the DOT1L inhibitor is SGC0946 and the Wnt agonist is AZD1080 and the second epigenetic agent is VPA. In some embodiments, the SGC0946 is administered, in an amount sufficient to achieve a concentration of about 10 nM, 20 nM, 30 nM, 40 nM, 50 nM, 60 nM, 70 nM, 80 nM, 90 nM, 100 nM, 200 nM, 300 nM, 400 nM, 500 nM, 600 nM, 700 nM, 800 nM, 900 nM, 1 μM, 2 μM, 3 μM, 4 μM, 5 μM, 6 μM, 7 μM, 8 μM, 9 μM, 10 μM, 11 μM, 12 μM, 13 μM, 14 μM, 15 μM, 20 μM, 25 μM, 30 μM, 35 μM, 40 μM, 45 μM, or about 50 μM in the perilymph fluid in the inner ear and AZD1080 is administered, in an amount sufficient to achieve a concentration of about is about 1 μM, 2 μM, 3 μM, 4 μM, 5 μM, 6 μM, 7 μM, 8 μM, 9 μM, or 10 μM in the perilymph fluid in the inner ear and VPA is administered in an amount sufficient to achieve a concentration of about is about 100 μM to 4 mM in the perilymph fluid in the inner ear. Alternatively, the SGC0946 is administered to a subject, for example, to the middle ear at a concentration of about 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, 10 mM, 11 mM, 12 mM, 13 mM, 14 mM, 15 mM, 20 mM, 25 mM, 30 mM, 35 mM, 40 mM, 45 mM, or about 50 mM and AZD1080 is administered to a subject, for example to the middle ear at a concentration of about 1 mM, 2 mM, 3 mM, 4 mM, 5 mM, 6 mM, 7 mM, 8 mM, 9 mM, or 10 mM and VPA is administered to a subject, for example, to the middle ear at a concentration about 100 mM to 4,000 mM.

In some embodiments the DOT1L inhibitor is SGC0946 and the Wnt agonist is LY209031 and the second epigenetic agent is VPA. In some embodiments, the SGC0946 is administered, in an amount sufficient to achieve a concentration of about 10 nM, 20 nM, 30 nM, 40 nM, 50 nM, 60 nM, 70 nM, 80 nM, 90 nM, 100 nM, 200 nM, 300 nM, 400 nM, 500 nM, 600 nM, 700 nM, 800 nM, 900 nM, 1 μM, 2 μM, 3 μM, 4 μM, 5 μM, 6 μM, 7 μM, 8 μM, 9 μM, 10 μM, 11 μM, 12 μM, 13 μM, 14 μM, 15 μM, 20 μM, 25 μM, 30 μM, 35 μM, 40 μM, 45 μM, or about 50 μM in the perilymph fluid in the inner ear and LY2090314 is administered in an amount sufficient to achieve a concentration of about 1 nM, 5 nM, 10 nM, 15 nM, 20 nM or 40 nM in the perilymph fluid in the inner ear and VPA is administered in an amount sufficient to achieve a concentration of about is about 100 μM to 4 mM in the perilymph fluid in the inner ear. Alternatively, the SGC0946 is administered to a subject, for example, to the middle ear at a concentration of about 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, 10 mM, 11 mM, 12 mM, 13 mM, 14 mM, 15 mM, 20 mM, 25 mM, 30 mM, 35 mM, 40 mM, 45 mM, or about 50 mM and LY2090314 is administered to a subject, for example, to the middle ear at a concentration of about 1 μM, 5 μM, 10 μM, 15 μM, or 20 μM and VPA is administered to a subject, for example to the middle ear at a concentration about 100 mM to 4,000 mM.

In some embodiments the DOT1L inhibitor is SGC0946 and the Wnt agonist is a substituted 3-imidazo[1,2-a]pyridin-3-yl-4-(1,2,3,4-tetrahydro-[1,4]diazepino-[6,7,1-hi]indol-7-yl)pyrrole-2,5-dione and the second epigenetic agent is VPA. In some embodiments, the SGC0946 is administered, in an amount sufficient to achieve a concentration of about 10 nM, 20 nM, 30 nM, 40 nM, 50 nM, 60 nM, 70 nM, 80 nM, 90 nM, 100 nM, 200 nM, 300 nM, 400 nM, 500 nM, 600 nM, 700 nM, 800 nM, 900 nM, 1 μM, 2 μM, 3 μM, 4 μM, 5 μM, 6 μM, 7 μM, 8 μM, 9 μM, 10 μM, 11 μM, 12 μM, 13 μM, 14 μM, 15 μM, 20 μM, 25 μM, 30 μM, 35 μM, 40 μM, 45 μM, or about 50 μM in the perilymph fluid in the inner ear and the substituted 3-Imidazo[1,2-a]pyridin-3-yl-4-(1,2,3,4-tetrahydro-[1,4]diazepino-[6,7,1-hi]indol-7-yl)pyrrole-2,5-dione is administered, in an amount sufficient to achieve a concentration of about 1 nM, 5 nM, 10 nM, 15 nM, 20 nM, 50 nM, 100 nM, 250 nM, or 500 nM, in the perilymph fluid in the inner ear and VPA is administered in an amount sufficient to achieve a concentration of about is about 100 μM to 4 mM in the perilymph fluid in the inner ear. Alternatively, the SGC0946 is administered to a subject, for example to the middle ear at a concentration of about 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, 10 mM, 11 mM, 12 mM, 13 mM, 14 mM, 15 mM, 20 mM, 25 mM, 30 mM, 35 mM, 40 mM, 45 mM, or about 50 mM and the substituted 3-imidazo[1,2-a]pyridin-3-yl-4-(1,2,3,4-tetrahydro-[1,4]diazepino-[6,7,1-hi]indol-7-yl)pyrrole-2,5-dione and is administered to a subject, for example to the middle ear at a concentration of about 1 μM, 5 μM, 10 μM, 15 μM, 20 μM, 50 μM, 100 μM, 250 μM, or 500 μM and VPA is administered to a subject, for example, to the middle ear at a concentration about 100 mM to 4,000 mM.

In some embodiments the DOT1L inhibitor is SGC0946 and the Wnt agonist is GSK3 inhibitor XXII and the second epigenetic agent is VPA. In some embodiments, the SGC0946 is administered, in an amount sufficient to achieve a concentration of about 10 nM, 20 nM, 30 nM, 40 nM, 50 nM, 60 nM, 70 nM, 80 nM, 90 nM, 100 nM, 200 nM, 300 nM, 400 nM, 500 nM, 600 nM, 700 nM, 800 nM, 900 nM, 1 μM, 2 μM, 3 μM, 4 μM, 5 μM, 6 μM, 7 μM, 8 μM, 9 μM, 10 μM, 11 μM, 12 μM, 13 μM, 14 μM, 15 μM, 20 μM, 25 μM, 30 μM, 35 μM, 40 μM, 45 μM, or about 50 μM in the perilymph fluid in the inner ear and GSK3-inhibitor XXII is administered, in an amount sufficient to achieve a concentration of about 0.1 μM, 0.2 μM, 0.3 μM, 0.4 μM, 0.5 μM, 0.6 μM, 0.7 μM, 0.8 μM, 0.9 μM, or 1.0 μM, in the perilymph fluid in the inner ear and VPA is administered in an amount sufficient to achieve a concentration of about is about 100 μM to 4 mM in the perilymph fluid in the inner ear. Alternatively, the SGC0946 is administered to a subject, for example, to the middle ear at a concentration of about 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, 10 mM, 11 mM, 12 mM, 13 mM, 14 mM, 15 mM, 20 mM, 25 mM, 30 mM, 35 mM, 40 mM, 45 mM, or about 50 mM and the GSK3-inhibitor XXII is administered, in an amount sufficient to achieve a concentration of about 0.1 mM, 0.2 mM, 0.3 mM, 0.4 mM, 0.5 mM, 0.6 mM, 0.7 mM, 0.8 mM, 0.9 mM, or 1.0 mM in the perilymph fluid in the inner ear and VPA is administered to a subject, for example, to the middle ear at a concentration about 100 mM to 4,000 mM.

In some embodiments the DOT1L inhibitor is SGC0946 and the Wnt agonist is CHIR99021 and the second epigenetic agent is VPA. In some embodiments, the SGC0946 is administered, in an amount sufficient to achieve a concentration of about 10 nM, 20 nM, 30 nM, 40 nM, 50 nM, 60 nM, 70 nM, 80 nM, 90 nM, 100 nM, 200 nM, 300 nM, 400 nM, 500 nM, 600 nM, 700 nM, 800 nM, 900 nM, 1 μM, 2 μM, 3 μM, 4 μM, 5 μM, 6 μM, 7 μM, 8 μM, 9 μM, 10 μM, 11 μM, 12 μM, 13 μM, 14 μM, 15 μM, 20 μM, 25 μM, 30 μM, 35 μM, 40 μM, 45 μM, or about 50 μM in the perilymph fluid in the inner ear and CHIR99021 is administered, in an amount sufficient to achieve a concentration of about 1 μM, 2 μM, 3 μM, 4 μM, 5 μM, 6 μM, 7 μM, 8 μM, 9 μM, or 10 μM, in the perilymph fluid in the inner ear and VPA is administered in an amount sufficient to achieve a concentration of about is about 100 μM to 4 mM in the perilymph fluid in the inner ear. Alternatively, the SGC0946 is administered to a subject, for example to the middle ear at a concentration of about 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, 10 mM, 11 mM, 12 mM, 13 mM, 14 mM, 15 mM, 20 mM, 25 mM, 30 mM, 35 mM, 40 mM, 45 mM, or about 50 mM and CHIR99021 is administered to a subject, for example, to the middle ear at a concentration of about 1 mM, 2 mM, 3 mM, 4 mM, 5 mM, 6 mM, 7 mM, 8 mM, 9 mM, or 10 mM and VPA is administered to a subject, for example, to the middle ear at a concentration about 100 mM to 4,000 mM.

In some embodiments the KDM inhibitor is TC-E 5002 and the Wnt agonist is AZD1080. In some embodiments, TC-E 5002 is administered, in amount sufficient to achieve a concentration of about 10 nM, 50 nM, 75 nM, 100 nM, 110 nM, 120 nM, 130 nM, 140 nM, 150 nM, 160 nM, 170 nM, 180 nM, 190 nM, 200 nM, 300 nM, 400 nM, 500 nM, 600 nM, 700 nM, 800 nM, 900 nM, 1 μM, 2 μM, 3 μM, 4 μM, 5 μM, 6 μM, 7 μM, 8 μM, 9 μM, or about 10 μM in the perilymph fluid in the inner ear and AZD1080 is administered, in amount sufficient to achieve a concentration of about is about 1 μM, 2 μM, 3 μM, 4 μM, 5 μM, 6 μM, 7 μM, 8 μM, 9 μM, or 10 μM, in the perilymph fluid in the inner ear. Alternatively, the TC-E 5002 is administered to a subject, for example to the middle ear at a concentration of about 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, or 10 mM and AZD1080, and is administered to a subject, for example to the middle ear at a concentration of about 1 mM, 2 mM, 3 mM, 4 mM, 5 mM, 6 mM, 7 mM, 8 mM, 9 mM, or 10 mM.

In some embodiments the KDM inhibitor is TC-E 5002 and the Wnt agonist is LY2090314. In some embodiments, the TC-E 5002 is administered, in amount sufficient to achieve a concentration of about 10 nM, 50 nM, 75 nM, 100 nM, 110 nM, 120 nM, 130 nM, 140 nM, 150 nM, 160 nM, 170 nM, 180 nM, 190 nM, 200 nM, 300 nM, 400 nM, 500 nM, 600 nM, 700 nM, 800 nM, 900 nM, 1 μM, 2 μM, 3 μM, 4 μM, 5 μM, 6 μM, 7 μM, 8 μM, 9 μM, or about 10 μM, in the perilymph fluid in the inner ear and LY2090314 is administered, in amount sufficient to achieve a concentration of about 1 nM, 5 nM, 10 nM, 15 nM, 20 nM, or 40 nM in the perilymph fluid in the inner ear. Alternatively, the TC-E 5002 is administered to a subject, for example to the middle ear at a concentration of about 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, or 10 mM and LY2090314, and is administered to a subject, for example to the middle ear at a concentration of about 1 μM, 5 μM, 10 μM, 15 μM, 20 μM, or 40 μM.

In some embodiments the KDM inhibitor is TC-E 5002 and the Wnt agonist is a substituted 3-Imidazo[1,2-a]pyridin-3-yl-4-(1,2,3,4-tetrahydro-[1,4]diazepino-[6,7,1-hi]indol-7-yl)pyrrole-2,5-dione. In some embodiments, the TC-E 5002 is administered, in amount sufficient to achieve a concentration of about 10 nM, 50 nM, 75 nM, 100 nM, 110 nM, 120 nM, 130 nM, 140 nM, 150 nM, 160 nM, 170 nM, 180 nM, 190 nM, 200 nM, 300 nM, 400 nM, 500 nM, 600 nM, 700 nM, 800 nM, 900 nM, 1 μM, 2 μM, 3 μM, 4 μM, 5 μM, 6 μM, 7 μM, 8 μM, 9 μM, or about 10 μM, in the perilymph fluid in the inner ear and the substituted 3-Imidazo[1,2-a]pyridin-3-yl-4-(1,2,3,4-tetrahydro-[1,4]diazepino-[6,7,1-hi]indol-7-yl)pyrrole-2,5-dione is administered, in amount sufficient to achieve a concentration of about 1 nM, 5 nM, 10 nM, 15 nM, 20 nM, 50 nM, 100 nM, 250 nM, or 500 nM, in the perilymph fluid in the inner ear. Alternatively, the AS TC-E 5002 is administered to a subject, for example to the middle ear at a concentration of about 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, or 10 mM and the substituted 3-Imidazo[1,2-a]pyridin-3-yl-4-(1,2,3,4-tetrahydro-[1,4]diazepino-[6,7,1-hi]indol-7-yl)pyrrole-2,5-dione and is administered to a subject, for example to the middle ear at a concentration of about 1 μM, 5 μM, 10 μM, 15 μM, 20 μM, 50 μM, 100 μM, 250 μM, or 500 μM.

In some embodiments the KDM inhibitor is TC-E 5002 and the Wnt agonist is GSK3 inhibitor XXII. In some embodiments, the TC-E 5002 is administered, in amount sufficient to achieve a concentration of about 10 nM, 50 nM, 75 nM, 100 nM, 110 nM, 120 nM, 130 nM, 140 nM, 150 nM, 160 nM, 170 nM, 180 nM, 190 nM, 200 nM, 300 nM, 400 nM, 500 nM, 600 nM, 700 nM, 800 nM, 900 nM, 1 μM, 2 μM, 3 μM, 4 μM, 5 μM, 6 μM, 7 μM, 8 μM, 9 μM, or about 10 μM, in the perilymph fluid in the inner ear and GSK3-inhibitor XXII is administered, in amount sufficient to achieve a concentration of about 0.1 μM, 0.2 μM, 0.3 μM, 0.4 μM, 0.5 μM, 0.6 μM, 0.7 μM, 0.8 μM, 0.9 μM, or 1.0 μM, in the perilymph fluid in the inner ear. Alternatively, the AS TC-E 5002 is administered to a subject, for example to the middle ear at a concentration of about 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, or 10 mM and the GSK3-inhibitor XXII is administered, in amount sufficient to achieve a concentration of about 0.1 mM, 0.2 mM, 0.3 mM, 0.4 mM, 0.5 mM, 0.6 mM, 0.7 mM, 0.8 mM, 0.9 mM, or 1.0 mM, in the perilymph fluid in the inner ear.

In some embodiments the KDM inhibitor is TC-E 5002 and the Wnt agonist is CHIR99021. In some embodiments, the TC-E 5002 is administered, in amount sufficient to achieve a concentration of about 10 nM, 50 nM, 75 nM, 100 nM, 110 nM, 120 nM, 130 nM, 140 nM, 150 nM, 160 nM, 170 nM, 180 nM, 190 nM, 200 nM, 300 nM, 400 nM, 500 nM, 600 nM, 700 nM, 800 nM, 900 nM, 1 μM, 2 μM, 3 μM, 4 μM, 5 μM, 6 μM, 7 μM, 8 μM, 9 μM, or about 10 μM, in the perilymph fluid in the inner ear and CHIR99021 is administered, in amount sufficient to achieve a concentration of about 1 μM, 2 μM, 3 μM, 4 μM, 5 μM, 6 μM, 7 μM, 8 μM, 9 μM, or 10 μM, in the perilymph fluid in the inner ear. Alternatively, the AS TC-E 5002 is administered to a subject, for example to the middle ear at a concentration of about 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, or 10 mM and CHIR99021 is administered to a subject, for example to the middle ear at a concentration of about 1 mM, 2 mM, 3 mM, 4 mM, 5 mM, 6 mM, 7 mM, 8 mM, 9 mM, or 10 mM.

In some embodiments the KDM inhibitor is AS 8351 and the Wnt agonist is AZD1080. In some embodiments, the AS 8351 is administered, in amount sufficient to achieve a concentration of about 10 nM, 20 nM, 30 nM, 40 nM, 50 nM, 60 nM, 70 nM, 80 nM, 90 nM, 100 nM, 200 nM, 300 nM, 400 nM, 500 nM, 600 nM, 700 nM, 800 nM, 900 nM, 1 μM, 2 μM, 3 μM, 4 μM, 5 μM, 6 μM, 7 μM, 8 μM, 9 μM, or about 10 μM, in the perilymph fluid in the inner ear in the perilymph fluid in the inner ear and AZD1080 is administered, in amount sufficient to achieve a concentration of about is about 1 μM, 2 μM, 3 μM, 4 μM, 5 μM, 6 μM, 7 μM, 8 μM, 9 μM, or 10 μM, in the perilymph fluid in the inner ear. Alternatively, the AS 8351 is administered to a subject, for example to the middle ear at a concentration of about 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, or 10 mM and AZD1080, and is administered to a subject, for example to the middle ear at a concentration of about 1 mM, 2 mM, 3 mM, 4 mM, 5 mM, 6 mM, 7 mM, 8 mM, 9 mM, or 10 mM.

In some embodiments the KDM inhibitor is AS 8351 and the Wnt agonist is LY2090314. In some embodiments, the AS 8351 is administered, in amount sufficient to achieve a concentration of about 10 nM, 20 nM, 30 nM, 40 nM, 50 nM, 60 nM, 70 nM, 80 nM, 90 nM, 100 nM, 200 nM, 300 nM, 400 nM, 500 nM, 600 nM, 700 nM, 800 nM, 900 nM, 1 μM, 2 μM, 3 μM, 4 μM, 5 μM, 6 μM, 7 μM, 8 μM, 9 μM, or about 10 μM, in the perilymph fluid in the inner ear and LY2090314 is administered, in amount sufficient to achieve a concentration of about 1 nM, 5 nM, 10 nM, 15 nM, 20 nM, or 40 nM in the perilymph fluid in the inner ear. Alternatively, the AS 8351 is administered to a subject, for example to the middle ear at a concentration of about 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, or 10 mM and LY2090314, and is administered to a subject, for example to the middle ear at a concentration of about 1 μM, 5 μM, 10 μM, 15 μM, 20 μM, or 40 μM.

In some embodiments the KDM inhibitor is AS 8351 and the Wnt agonist is a substituted 3-Imidazo[1,2-a]pyridin-3-yl-4-(1,2,3,4-tetrahydro-[1,4]diazepino-[6,7,1-hi]indol-7-yl)pyrrole-2,5-dione. In some embodiments, the AS 8351 is administered, in amount sufficient to achieve a concentration of about 10 nM, 20 nM, 30 nM, 40 nM, 50 nM, 60 nM, 70 nM, 80 nM, 90 nM, 100 nM, 200 nM, 300 nM, 400 nM, 500 nM, 600 nM, 700 nM, 800 nM, 900 nM, 1 μM, 2 μM, 3 μM, 4 μM, 5 μM, 6 μM, 7 μM, 8 μM, 9 μM, or about 10 μM, in the perilymph fluid in the inner ear and the substituted 3-Imidazo[1,2-a]pyridin-3-yl-4-(1,2,3,4-tetrahydro-[1,4]diazepino-[6,7,1-hi]indol-7-yl)pyrrole-2,5-dione is administered, in amount sufficient to achieve a concentration of about 1 nM, 5 nM, 10 nM, 15 nM, 20 nM, 50 nM, 100 nM, 250 nM, or 500 nM, in the perilymph fluid in the inner ear. Alternatively, the AS 8351 is administered to a subject, for example to the middle ear at a concentration of about 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, or 10 mM and the substituted 3-Imidazo[1,2-a]pyridin-3-yl-4-(1,2,3,4-tetrahydro-[1,4]diazepino-[6,7,1-hi]indol-7-yl)pyrrole-2,5-dione and is administered to a subject, for example to the middle ear at a concentration of about 1 μM, 5 μM, 10 μM, 15 μM, 20 μM, 50 μM, 100 μM, 250 μM, or 500 μM.

In some embodiments the KDM inhibitor is AS 8351 and the Wnt agonist is GSK3 inhibitor XXII. In some embodiments, the AS 8351 is administered, in amount sufficient to achieve a concentration of about 10 nM, 20 nM, 30 nM, 40 nM, 50 nM, 60 nM, 70 nM, 80 nM, 90 nM, 100 nM, 200 nM, 300 nM, 400 nM, 500 nM, 600 nM, 700 nM, 800 nM, 900 nM, 1 μM, 2 μM, 3 μM, 4 μM, 5 μM, 6 μM, 7 μM, 8 μM, 9 μM, or about 10 μM, in the perilymph fluid in the inner ear and GSK3-inhibitor XXII is administered, in amount sufficient to achieve a concentration of about 0.1 μM, 0.2 μM, 0.3 μM, 0.4 μM, 0.5 μM, 0.6 μM, 0.7 μM, 0.8 μM, 0.9 μM, or 1.0 μM, in the perilymph fluid in the inner ear. Alternatively, the AS 8351 is administered to a subject, for example to the middle ear at a concentration of about 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, or 10 mM and the GSK3-inhibitor XXII is administered, in amount sufficient to achieve a concentration of about 0.1 mM, 0.2 mM, 0.3 mM, 0.4 mM, 0.5 mM, 0.6 mM, 0.7 mM, 0.8 mM, 0.9 mM, or 1.0 mM, in the perilymph fluid in the inner ear.

In some embodiments the KDM inhibitor is AS 8351 and the Wnt agonist is CHIR99021. In some embodiments, the AS 8351 is administered, in amount sufficient to achieve a concentration of about 10 nM, 20 nM, 30 nM, 40 nM, 50 nM, 60 nM, 70 nM, 80 nM, 90 nM, 100 nM, 200 nM, 300 nM, 400 nM, 500 nM, 600 nM, 700 nM, 800 nM, 900 nM, 1 μM, 2 μM, 3 μM, 4 μM, 5 μM, 6 μM, 7 μM, 8 μM, 9 μM, or about 10 μM, in the perilymph fluid in the inner ear and CHIR99021 is administered, in amount sufficient to achieve a concentration of about 1 μM, 2 μM, 3 μM, 4 μM, 5 μM, 6 μM, 7 μM, 8 μM, 9 μM, or 10 μM, in the perilymph fluid in the inner ear. Alternatively, the AS 8351 is administered to a subject, for example to the middle ear at a concentration of about 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, or 10 mM and CHIR99021 is administered to a subject, for example to the middle ear at a concentration of about 1 mM, 2 mM, 3 mM, 4 mM, 5 mM, 6 mM, 7 mM, 8 mM, 9 mM, or 10 mM.

In some embodiments the KDM inhibitor is EPT-103182 and the Wnt agonist is AZD1080. In some embodiments, the EPT-103182 is administered, in amount sufficient to achieve a concentration of about 0.1 nM, 0.2 nM, 0.3 nM, 0.4 nM, 0.5 nM, 0.6 nM, 0.7 nM, 0.8 nM, 0.9 nM, 1.0 nM, 2.0 nM, 3.0 nM, 4.0 nM, 5.0 nM, 6.0 nM, 7.0 nM, 8.0 nM, 9.0 nM, 10 nM, 20 nM, 30 nM, 40 nM, 50 nM, 60 nM, 70 nM, 80 nM, 90 nM, 100 nM, 200 nM, 300 nM, 400 nM, 500 nM, 600 nM, 700 nM, 800 nM, 900 nM, or about 1 μM in the perilymph fluid in the inner ear and AZD1080 is administered, in amount sufficient to achieve a concentration of about is about 1 μM, 2 μM, 3 μM, 4 μM, 5 μM, 6 μM, 7 μM, 8 μM, 9 μM, or 10 μM, in the perilymph fluid in the inner ear. Alternatively, the EPT-103182 is administered to a subject, for example to the middle ear at a concentration of about 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, or about 1 mM and AZD1080, and is administered to a subject, for example to the middle ear at a concentration of about 1 mM, 2 mM, 3 mM, 4 mM, 5 mM, 6 mM, 7 mM, 8 mM, 9 mM, or 10 mM.

In some embodiments the KDM inhibitor is EPT-103182 and the Wnt agonist is LY2090314. In some embodiments, the EPT-103182 is administered, in amount sufficient to achieve a concentration of about 0.1 nM, 0.2 nM, 0.3 nM, 0.4 nM, 0.5 nM, 0.6 nM, 0.7 nM, 0.8 nM, 0.9 nM, 1.0 nM, 2.0 nM, 3.0 nM, 4.0 nM, 5.0 nM, 6.0 nM, 7.0 nM, 8.0 nM, 9.0 nM, 10 nM, 20 nM, 30 nM, 40 nM, 50 nM, 60 nM, 70 nM, 80 nM, 90 nM, 100 nM, 200 nM, 300 nM, 400 nM, 500 nM, 600 nM, 700 nM, 800 nM, 900 nM, or about 1 μM in the perilymph fluid in the inner ear and LY2090314 is administered, in amount sufficient to achieve a concentration of about 1 nM, 5 nM, 10 nM, 15 nM, 20 nM, or 40 nM in the perilymph fluid in the inner ear. Alternatively, the EPT-103182 is administered to a subject, for example to the middle ear at a concentration of about 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, or about 1 mM and LY2090314, and is administered to a subject, for example to the middle ear at a concentration of about 1 μM, 5 μM, 10 μM, 15 μM, 20 μM, or 40 μM.

In some embodiments the KDM inhibitor is EPT-103182 and the Wnt agonist is a substituted 3-Imidazo[1,2-a]pyridin-3-yl-4-(1,2,3,4-tetrahydro-[1,4]diazepino-[6,7,1-hi]indol-7-yl)pyrrole-2,5-dione. In some embodiments, the EPT-103182 is administered, in amount sufficient to achieve a concentration of about 0.1 nM, 0.2 nM, 0.3 nM, 0.4 nM, 0.5 nM, 0.6 nM, 0.7 nM, 0.8 nM, 0.9 nM, 1.0 nM, 2.0 nM, 3.0 nM, 4.0 nM, 5.0 nM, 6.0 nM, 7.0 nM, 8.0 nM, 9.0 nM, 10 nM, 20 nM, 30 nM, 40 nM, 50 nM, 60 nM, 70 nM, 80 nM, 90 nM, 100 nM, 200 nM, 300 nM, 400 nM, 500 nM, 600 nM, 700 nM, 800 nM, 900 nM, or about 1 μM in the perilymph fluid in the inner ear and the substituted 3-Imidazo[1,2-a]pyridin-3-yl-4-(1,2,3,4-tetrahydro-[1,4 ]diazepino-[6,7,1-hi]indol-7-yl)pyrrole-2,5-dione is administered, in amount sufficient to achieve a concentration of about 1 nM, 5 nM, 10 nM, 15 nM, 20 nM, 50 nM, 100 nM, 250 nM, or 500 nM, in the perilymph fluid in the inner ear. Alternatively, the EPT-103182 is administered to a subject, for example to the middle ear at a concentration of about 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, or about 1 mM and the substituted 3-Imidazo[1,2-a]pyridin-3-yl-4-(1,2,3,4-tetrahydro-[1,4]diazepino-[6,7,1-hi]indol-7-yl)pyrrole-2,5-dione and is administered to a subject, for example to the middle ear at a concentration of about 1 μM, 5 μM, 10 μM, 15 μM, 20 μM, 50 μM, 100 μM, 250 μM, or 500 μM.

In some embodiments the KDM inhibitor is EPT-103182 and the Wnt agonist is GSK3 inhibitor XXII. In some embodiments, the EFT-103182 is administered, in amount sufficient to achieve a concentration of about 0.1 nM, 0.2 nM, 0.3 nM, 0.4 nM, 0.5 nM, 0.6 nM, 0.7 nM, 0.8 nM, 0.9 nM, 1.0 nM, 2.0 nM, 3.0 nM, 4.0 nM, 5.0 nM, 6.0 nM, 7.0 nM, 8.0 nM, 9.0 nM, 10 nM, 20 nM, 30 nM, 40 nM, 50 nM, 60 nM, 70 nM, 80 nM, 90 nM, 100 nM, 200 nM, 300 nM, 400 nM, 500 nM, 600 nM, 700 nM, 800 nM, 900 nM, or about 1 μM in the perilymph fluid in the inner ear and GSK3-inhibitor XXII is administered, in amount sufficient to achieve a concentration of about 0.1 μM, 0.2 μM, 0.3 μM, 0.4 μM, 0.5 μM, 0.6 μM, 0.7 μM, 0.8 μM, 0.9 μM, or 1.0 μM, in the perilymph fluid in the inner ear. Alternatively, the EPT-103182 is administered to a subject, for example to the middle ear at a concentration of about 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, or about 1 mM and the GSK3-inhibitor XXII is administered, in amount sufficient to achieve a concentration of about 0.1 mM, 0.2 mM, 0.3 mM, 0.4 mM, 0.5 mM, 0.6 mM, 0.7 mM, 0.8 mM, 0.9 mM, or 1.0 mM, in the perilymph fluid in the inner ear.

In some embodiments the KDM inhibitor is EPT-103182 and the Wnt agonist is CHIR99021. In some embodiments, the EPT-103182 is administered, in amount sufficient to achieve a concentration of about 0.1 nM, 0.2 nM, 0.3 nM, 0.4 nM, 0.5 nM, 0.6 nM, 0.7 nM, 0.8 nM, 0.9 nM, 1.0 nM, 2.0 nM, 3.0 nM, 4.0 nM, 5.0 nM, 6.0 nM, 7.0 nM, 8.0 nM, 9.0 nM, 10 nM, 20 nM, 30 nM, 40 nM, 50 nM, 60 nM, 70 nM, 80 nM, 90 nM, 100 nM, 200 nM, 300 nM, 400 nM, 500 nM, 600 nM, 700 nM, 800 nM, 900 nM, or about 1 μM in the perilymph fluid in the inner ear and CHIR99021 is administered, in amount sufficient to achieve a concentration of about 1 μM, 2 μM, 3 μM, 4 μM, 5 μM, 6 μM, 7 μM, 8 μM, 9 μM, or 10 μM, in the perilymph fluid in the inner ear. Alternatively, the EPT-103182 is administered to a subject, for example to the middle ear at a concentration of about 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, or about 1 mM and CHIR99021 is administered to a subject, for example to the middle ear at a concentration of about 1 mM, 2 mM, 3 mM, 4 mM, 5 mM, 6 mM, 7 mM, 8 mM, 9 mM, or 10 mM.

In some embodiments the KDM inhibitor is TC-E 5002; the Wnt agonist is AZD1080 and the second epigenetic agent is VPA. In some embodiments, TC-E 5002 is administered, in amount sufficient to achieve a concentration of about 10 nM, 50 nM, 75 nM, 100 nM, 110 nM, 120 nM, 130 nM, 140 nM, 150 nM, 160 nM, 170 nM, 180 nM, 190 nM, 200 nM, 300 nM, 400 nM, 500 nM, 600 nM, 700 nM, 800 nM, 900 nM, 1 μM, 2 μM, 3 μM, 4 μM, 5 μM, 6 μM, 7 μM, 8 μM, 9 μM, or about 10 μM in the perilymph fluid in the inner ear; AZD1080 is administered, in amount sufficient to achieve a concentration of about is about 1 μM, 2 μM, 3 μM, 4 μM, 5 μM, 6 μM, 7 μM, 8 μM, 9 μM, or 10 μM, in the perilymph fluid in the inner ear and VPA is administered in amount sufficient to achieve a concentration of about is about 100 μM to 4 mM in the perilymph fluid in the inner ear. Alternatively, the TC-E 5002 is administered to a subject, for example to the middle ear at a concentration of about 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, or 10 mM; AZD1080, and is administered to a subject, for example to the middle ear at a concentration of about 1 mM, 2 mM, 3 mM, 4 mM, 5 mM, 6 mM, 7 mM, 8 mM, 9 mM, or 10 mM and VPA to a subject, for example to the middle ear at a concentration about 100 mM to 4,000 mM.

In some embodiments the KDM inhibitor is TC-E 5002; the Wnt agonist is LY2090314 and the second epigenetic agent is VPA. In some embodiments, the TC-E 5002 is administered, in amount sufficient to achieve a concentration of about 10 nM, 50 nM, 75 nM, 100 nM, 110 nM, 120 nM, 130 nM, 140 nM, 150 nM, 160 nM, 170 nM, 180 nM, 190 nM, 200 nM, 300 nM, 400 nM, 500 nM, 600 nM, 700 nM, 800 nM, 900 nM, 1 μM, 2 μM, 3 μM, 4 μM, 5 μM, 6 μM, 7 μM, 8 μM, 9 μM, or about 10 μM, in the perilymph fluid in the inner ear; LY2090314 is administered, in amount sufficient to achieve a concentration of about 1 nM, 5 nM, 10 nM, 15 nM, 20 nM, or 40 nM in the perilymph fluid in the inner ear and VPA is administered in amount sufficient to achieve a concentration of about is about 100 μM to 4 mM in the perilymph fluid in the inner ear. Alternatively, the TC-E 5002 is administered to a subject, for example to the middle ear at a concentration of about 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, or 10 mM; LY2090314, and is administered to a subject, for example to the middle ear at a concentration of about 1 μM, 5 μM, 10 μM, 15 μM, 20 μM, or 40 μM and VPA to a subject, for example to the middle ear at a concentration about 100 mM to 4,000 mM.

In some embodiments the KDM inhibitor is TC-E 5002; the Wnt agonist is a substituted 3-Imidazo[1,2-a]pyridin-3-yl-4-(1,2,3,4-tetrahydro-[1,4]diazepino-[6,7,1-hi]indol-7-yl)pyrrole-2,5-dione and the second epigenetic agent is VPA. In some embodiments, the TC-E 5002 is administered, in amount sufficient to achieve a concentration of about 10 nM, 50 nM, 75 nM, 100 nM, 110 nM, 120 nM, 130 nM, 140 nM, 150 nM, 160 nM, 170 nM, 180 nM, 190 nM, 200 nM, 300 nM, 400 nM, 500 nM, 600 nM, 700 nM, 800 nM, 900 nM, 1 μM, 2 μM, 3 μM, 4 μM, 5 μM, 6 μM, 7 μM, 8 μM, 9 μM, or about 10 μM, in the perilymph fluid in the inner ear; the substituted 3-Imidazo[1,2-a]pyridin-3-yl-4-(1,2,3,4-tetrahydro-[1,4]diazepino-[6,7,1-hi]indol-7-yl)pyrrole-2,5-dione is administered, in amount sufficient to achieve a concentration of about 1 nM, 5 nM, 10 nM, 15 nM, 20 nM, 50 nM, 100 nM, 250 nM, or 500 nM, in the perilymph fluid in the inner ear and VPA is administered in amount sufficient to achieve a concentration of about is about 100 μM to 4 mM in the perilymph fluid in the inner ear. Alternatively, the AS TC-E 5002 is administered to a subject, for example to the middle ear at a concentration of about 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, or 10 mM; the substituted 3-Imidazo[1,2-a]pyridin-3-yl-4-(1,2,3,4-tetrahydro-[1,4]diazepino-[6,7,1-hi]indol-7-yl)pyrrole-2,5-dione and is administered to a subject, for example to the middle ear at a concentration of about 1 μM, 5 μM, 10 μM, 15 μM, 20 μM, 50 μM, 100 μM, 250 μM, or 500 μM and VPA to a subject, for example to the middle ear at a concentration about 100 mM to 4,000 mM.

In some embodiments the KDM inhibitor is TC-E 5002; the Wnt agonist is GSK3 inhibitor XXII and the second epigenetic agent is VPA. In some embodiments, the TC-E 5002 is administered, in amount sufficient to achieve a concentration of about 10 nM, 50 nM, 75 nM, 100 nM, 110 nM, 120 nM, 130 nM, 140 nM, 150 nM, 160 nM, 170 nM, 180 nM, 190 nM, 200 nM, 300 nM, 400 nM, 500 nM, 600 nM, 700 nM, 800 nM, 900 nM, 1 μM, 2 μM, 3 μM, 4 μM, 5 μM, 6 μM, 7 μM, 8 μM, 9 μM, or about 10 μM, in the perilymph fluid in the inner ear; GSK3-inhibitor XXII is administered, in amount sufficient to achieve a concentration of about 0.1 μM, 0.2 μM, 0.3 μM, 0.4 μM, 0.5 μM, 0.6 μM, 0.7 μM, 0.8 μM, 0.9 μM, or 1.0 μM, in the perilymph fluid in the inner ear and VPA is administered in amount sufficient to achieve a concentration of about is about 100 μM to 4 mM in the perilymph fluid in the inner ear. Alternatively, the AS TC-E 5002 is administered to a subject, for example to the middle ear at a concentration of about 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, or 10 mM; the GSK3-inhibitor XXII is administered, in amount sufficient to achieve a concentration of about 0.1 mM, 0.2 mM, 0.3 mM, 0.4 mM, 0.5 mM, 0.6 mM, 0.7 mM, 0.8 mM, 0.9 mM, or 1.0 mM, in the perilymph fluid in the inner ear and VPA to a subject, for example to the middle ear at a concentration about 100 mM to 4,000 mM.

In some embodiments the KDM inhibitor is TC-E 5002; the Wnt agonist is CHIR99021 and the second epigenetic agent is VPA. In some embodiments, the TC-E 5002 is administered, in amount sufficient to achieve a concentration of about 10 nM, 50 nM, 75 nM, 100 nM, 110 nM, 120 nM, 130 nM, 140 nM, 150 nM, 160 nM, 170 nM, 180 nM, 190 nM, 200 nM, 300 nM, 400 nM, 500 nM, 600 nM, 700 nM, 800 nM, 900 nM, 1 μM, 2 μM, 3 μM, 4 μM, 5 μM, 6 μM, 7 μM, 8 μM, 9 μM, or about 10 μM, in the perilymph fluid in the inner ear; CHIR99021 is administered, in amount sufficient to achieve a concentration of about 1 μM, 2 μM, 3 μM, 4 μM, 5 μM, 6 μM, 7 μM, 8 μM, 9 μM, or 10 μM, in the perilymph fluid in the inner ear and VPA is administered in amount sufficient to achieve a concentration of about is about 100 μM to 4 mM in the perilymph fluid in the inner ear. Alternatively, the AS TC-E 5002 is administered to a subject, for example to the middle ear at a concentration of about 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, or 10 mM; CHIR99021 is administered to a subject, for example to the middle ear at a concentration of about 1 mM, 2 mM, 3 mM, 4 mM, 5 mM, 6 mM, 7 mM, 8 mM, 9 mM, or 10 mM and VPA to a subject, for example to the middle ear at a concentration about 100 mM to 4,000 mM.

In some embodiments the KDM inhibitor is AS 8351; the Wnt agonist is AZD1080 and the second epigenetic agent is VPA. In some embodiments, the AS 8351 is administered, in amount sufficient to achieve a concentration of about 10 nM, 20 nM, 30 nM, 40 nM, 50 nM, 60 nM, 70 nM, 80 nM, 90 nM, 100 nM, 200 nM, 300 nM, 400 nM, 500 nM, 600 nM, 700 nM, 800 nM, 900 nM, 1 μM, 2 μM, 3 μM, 4 μM, 5 μM, 6 μM, 7 μM, 8 μM, 9 μM, or about 10 μM, in the perilymph fluid in the inner ear; AZD1080 is administered, in amount sufficient to achieve a concentration of about is about 1 μM, 2 μM, 3 μM, 4 μM, 5 μM, 6 μM, 7 μM, 8 μM, 9 μM, or 10 μM, in the perilymph fluid in the inner ear and VPA is administered in amount sufficient to achieve a concentration of about is about 100 μM to 4 mM in the perilymph fluid in the inner ear. Alternatively, the AS 8351 is administered to a subject, for example to the middle ear at a concentration of about 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, or 10 mM; AZD1080, and is administered to a subject, for example to the middle ear at a concentration of about 1 mM, 2 mM, 3 mM, 4 mM, 5 mM, 6 mM, 7 mM, 8 mM, 9 mM, or 10 mM and VPA to a subject, for example to the middle ear at a concentration about 100 mM to 4,000 mM.

In some embodiments the KDM inhibitor is AS 8351, the Wnt agonist is LY2090314 and the second epigenetic agent is VPA. In some embodiments, the AS 8351 is administered, in amount sufficient to achieve a concentration of about 10 nM, 20 nM, 30 nM, 40 nM, 50 nM, 60 nM, 70 nM, 80 nM, 90 nM, 100 nM, 200 nM, 300 nM, 400 nM, 500 nM, 600 nM, 700 nM, 800 nM, 900 nM, 1 μM, 2 μM, 3 μM, 4 μM, 5 μM, 6 μM, 7 μM, 8 μM, 9 μM, or about 10 μM, in the perilymph fluid in the inner ear; LY2090314 is administered, in amount sufficient to achieve a concentration of about 1 nM, 5 nM, 10 nM, 15 nM, 20 nM, or 40 nM in the perilymph fluid in the inner ear and VPA is administered in amount sufficient to achieve a concentration of about is about 100 μM to 4 mM in the perilymph fluid in the inner ear. Alternatively, the AS 8351 is administered to a subject, for example to the middle ear at a concentration of about 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, or 10 mM; LY2090314, and is administered to a subject, for example to the middle ear at a concentration of about 1 μM, 5 μM, 10 μM, 15 μM, 20 μM, or 40 μM and VPA to a subject, for example to the middle ear at a concentration about 100 mM to 4,000 mM.

In some embodiments the KDM inhibitor is AS 8351, the Wnt agonist is a substituted 3-Imidazo[1,2-a]pyridin-3-yl-4-(1,2,3,4-tetrahydro-[1,4]diazepino-[6,7,1-hi]indol-7-yl)pyrrole-2,5-dione and the second epigenetic agent is VPA. In some embodiments, the AS 8351 is administered, in amount sufficient to achieve a concentration of about 10 nM, 20 nM, 30 nM, 40 nM, 50 nM, 60 nM, 70 nM, 80 nM, 90 nM, 100 nM, 200 nM, 300 nM, 400 nM, 500 nM, 600 nM, 700 nM, 800 nM, 900 nM, 1 μM, 2 μM, 3 μM, 4 μM, 5 μM, 6 μM, 7 μM, 8 μM, 9 μM, or about 10 μM, in the perilymph fluid in the inner ear; the substituted 3-Imidazo[1,2-a]pyridin-3-yl-4-(1,2,3,4-tetrahydro-[1,4]diazepino-[6,7,1-hi]indol-7-yl)pyrrole-2,5-dione is administered, in amount sufficient to achieve a concentration of about 1 nM, 5 nM, 10 nM, 15 nM, 20 nM, 50 nM, 100 nM, 250 nM, or 500 nM, in the perilymph fluid in the inner ear and VPA is administered in amount sufficient to achieve a concentration of about is about 100 μM to 4 mM in the perilymph fluid in the inner ear. Alternatively, the AS 8351 is administered to a subject, for example to the middle ear at a concentration of about 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, or 10 mM; the substituted 3-Imidazo[1,2-a]pyridin-3-yl-4-(1,2,3,4-tetrahydro-[1,4]diazepino-[6,7,1-hi]indol-7-yl)pyrrole-2,5-dione and is administered to a subject, for example to the middle ear at a concentration of about 1 μM, 5 μM, 10 μM, 15 μM, 20 μM, 50 μM, 100 μM, 250 μM, or 500 μM and VPA to a subject, for example to the middle ear at a concentration about 100 mM to 4,000 mM.

In some embodiments the KDM inhibitor is AS 8351, the Wnt agonist is GSK3 inhibitor XXII and the second epigenetic agent is VPA. In some embodiments, the AS 8351 is administered, in amount sufficient to achieve a concentration of about 10 nM, 20 nM, 30 nM, 40 nM, 50 nM, 60 nM, 70 nM, 80 nM, 90 nM, 100 nM, 200 nM, 300 nM, 400 nM, 500 nM, 600 nM, 700 nM, 800 nM, 900 nM, 1 μM, 2 μM, 3 μM, 4 μM, 5 μM, 6 μM, 7 μM, 8 μM, 9 μM, or about 10 μM, in the perilymph fluid in the inner ear; GSK3-inhibitor XXII is administered, in amount sufficient to achieve a concentration of about 0.1 μM, 0.2 μM, 0.3 μM, 0.4 μM, 0.5 μM, 0.6 μM, 0.7 μM, 0.8 μM, 0.9 μM, or 1.0 μM, in the perilymph fluid in the inner ear and VPA is administered in amount sufficient to achieve a concentration of about is about 100 μM to 4 mM in the perilymph fluid in the inner ear. Alternatively, the AS 8351 is administered to a subject, for example to the middle ear at a concentration of about 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, or 10 mM; the GSK3-inhibitor XXII is administered, in amount sufficient to achieve a concentration of about 0.1 mM, 0.2 mM, 0.3 mM, 0.4 mM, 0.5 mM, 0.6 mM, 0.7 mM, 0.8 mM, 0.9 mM, or 1.0 mM, in the perilymph fluid in the inner ear and VPA to a subject, for example to the middle ear at a concentration about 100 mM to 4,000 mM.

In some embodiments the KDM inhibitor is AS 8351; the Wnt agonist is CHIR99021 and the second epigenetic agent is VPA. In some embodiments, the AS 8351 is administered, in amount sufficient to achieve a concentration of about 10 nM, 20 nM, 30 nM, 40 nM, 50 nM, 60 nM, 70 nM, 80 nM, 90 nM, 100 nM, 200 nM, 300 nM, 400 nM, 500 nM, 600 nM, 700 nM, 800 nM, 900 nM, 1 μM, 2 μM, 3 μM, 4 μM, 5 μM, 6 μM, 7 μM, 8 μM, 9 μM, or about 10 μM, in the perilymph fluid in the inner ear; CHIR99021 is administered, in amount sufficient to achieve a concentration of about 1 μM, 2 μM, 3 μM, 4 μM, 5 μM, 6 μM, 7 μM, 8 μM, 9 μM, or 10 μM, in the perilymph fluid in the inner ear and VPA is administered in amount sufficient to achieve a concentration of about is about 100 μM to 4 mM in the perilymph fluid in the inner ear. Alternatively, the AS 8351 is administered to a subject, for example to the middle ear at a concentration of about 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, or 10 mM; CHIR99021 is administered to a subject, for example to the middle ear at a concentration of about 1 mM, 2 mM, 3 mM, 4 mM, 5 mM, 6 mM, 7 mM, 8 mM, 9 mM, or 10 mM and VPA to a subject, for example to the middle ear at a concentration about 100 mM to 4,000 mM.

In some embodiments the KDM inhibitor is EPT-103182, the Wnt agonist is AZD1080 and the second epigenetic agent is VPA. In some embodiments, the EPT-103182 is administered, in amount sufficient to achieve a concentration of about 0.1 nM, 0.2 nM, 0.3 nM, 0.4 nM, 0.5 nM, 0.6 nM, 0.7 nM, 0.8 nM, 0.9 nM, 1.0 nM, 2.0 nM, 3.0 nM, 4.0 nM, 5.0 nM, 6.0 nM, 7.0 nM, 8.0 nM, 9.0 nM, 10 nM, 20 nM, 30 nM, 40 nM, 50 nM, 60 nM, 70 nM, 80 nM, 90 nM, 100 nM, 200 nM, 300 nM, 400 nM, 500 nM, 600 nM, 700 nM, 800 nM, 900 nM, or about 1 μM in the perilymph fluid in the inner ear; AZD1080 is administered, in amount sufficient to achieve a concentration of about is about 1 μM, 2 μM, 3 μM, 4 μM, 5 μM, 6 μM, 7 μM, 8 μM, 9 μM, or 10 μM, in the perilymph fluid in the inner ear and VPA is administered in amount sufficient to achieve a concentration of about is about 100 μM to 4 mM in the perilymph fluid in the inner ear. Alternatively, the EPT-103182 is administered to a subject, for example to the middle ear at a concentration of about 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, or about 1 mM; AZD1080, and is administered to a subject, for example to the middle ear at a concentration of about 1 mM, 2 mM, 3 mM, 4 mM, 5 mM, 6 mM, 7 mM, 8 mM, 9 mM, or 10 mM and VPA to a subject, for example to the middle ear at a concentration about 100 mM to 4,000 mM.

In some embodiments the KDM inhibitor is EPT-103182; the Wnt agonist is LY2090314 and the second epigenetic agent is VPA. In some embodiments, the EPT-103182 is administered, in amount sufficient to achieve a concentration of about 0.1 nM, 0.2 nM, 0.3 nM, 0.4 nM, 0.5 nM, 0.6 nM, 0.7 nM, 0.8 nM, 0.9 nM, 1.0 nM, 2.0 nM, 3.0 nM, 4.0 nM, 5.0 nM, 6.0 nM, 7.0 nM, 8.0 nM, 9.0 nM, 10 nM, 20 nM, 30 nM, 40 nM, 50 nM, 60 nM, 70 nM, 80 nM, 90 nM, 100 nM, 200 nM, 300 nM, 400 nM, 500 nM, 600 nM, 700 nM, 800 nM, 900 nM, or about 1 μM in the perilymph fluid in the inner ear; LY2090314 is administered, in amount sufficient to achieve a concentration of about 1 nM, 5 nM, 10 nM, 15 nM, 20 nM, or 40 nM in the perilymph fluid in the inner ear and VPA is administered in amount sufficient to achieve a concentration of about is about 100 μM to 4 mM in the perilymph fluid in the inner ear. Alternatively, the EPT-103182 is administered to a subject, for example to the middle ear at a concentration of about 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, or about 1 mM; LY2090314, and is administered to a subject, for example to the middle ear at a concentration of about 1 μM, 5 μM, 10 μM, 15 μM, 20 μM, or 40 μM and VPA to a subject, for example to the middle ear at a concentration about 100 mM to 4,000 mM.

In some embodiments the KDM inhibitor is EPT-103182; the Wnt agonist is a substituted 3-Imidazo[1,2-a]pyridin-3-yl-4-(1,2,3,4-tetrahydro-[1,4]diazepino-[6,7,1-hi]indol-7-yl)pyrrole-2,5-dione and the second epigenetic agent is VPA. In some embodiments, the EPT-103182 is administered, in amount sufficient to achieve a concentration of about 0.1 nM, 0.2 nM, 0.3 nM, 0.4 nM, 0.5 nM, 0.6 nM, 0.7 nM, 0.8 nM, 0.9 nM, 1.0 nM, 2.0 nM, 3.0 nM, 4.0 nM, 5.0 nM, 6.0 nM, 7.0 nM, 8.0 nM, 9.0 nM, 10 nM, 20 nM, 30 nM, 40 nM, 50 nM, 60 nM, 70 nM, 80 nM, 90 nM, 100 nM, 200 nM, 300 nM, 400 nM, 500 nM, 600 nM, 700 nM, 800 nM, 900 nM, or about 1 μM in the perilymph fluid in the inner ear; the substituted 3-Imidazo[1,2-a]pyridin-3-yl-4-(1,2,3,4-tetrahydro-[1,4]diazepino-[6,7,1-hi]indol-7-yl)pyrrole-2,5-dione is administered, in amount sufficient to achieve a concentration of about 1 nM, 5 nM, 10 nM, 15 nM, 20 nM, 50 nM, 100 nM, 250 nM, or 500 nM, in the perilymph fluid in the inner ear and VPA is administered in amount sufficient to achieve a concentration of about is about 100 μM to 4 mM in the perilymph fluid in the inner ear. Alternatively, the EPT-103182 is administered to a subject, for example to the middle ear at a concentration of about 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, or about 1 mM; the substituted 3-Imidazo[1,2-a]pyridin-3-yl-4-(1,2,3,4-tetrahydro-[1,4]diazepino-[6,7,1-hr]indol-7-yl)pyrrole-2,5-dione and is administered to a subject, for example to the middle ear at a concentration of about 1 μM, 5 μM, 10 μM, 15 μM, 20 μM, 50 μM, 100 μM, 250 μM, or 500 μM and VPA to a subject, for example to the middle ear at a concentration about 100 mM to 4,000 mM.

In some embodiments the KDM inhibitor is EPT-103182, the Wnt agonist is GSK3 inhibitor XXII and the second epigenetic agent is VPA. In some embodiments, the EPT-103182 is administered, in amount sufficient to achieve a concentration of about 0.1 nM, 0.2 nM, 0.3 nM, 0.4 mM, 0.5 nM, 0.6 nM, 0.7 nM, 0.8 nM, 0.9 nM, 1.0 nM, 2.0 nM, 3.0 nM, 4.0 nM, 5.0 nM, 6.0 nM, 7.0 nM, 8.0 nM, 9.0 nM, 10 nM, 20 nM, 30 nM, 40 nM, 50 nM, 60 nM, 70 nM, 80 nM, 90 nM, 100 nM, 200 nM, 300 nM, 400 nM, 500 nM, 600 nM, 700 nM, 800 nM, 900 nM, or about 1 μM in the perilymph fluid in the inner ear; GSK3-inhibitor XXII is administered, in amount sufficient to achieve a concentration of about 0.1 μM, 0.2 μM, 0.3 μM, 0.4 μM, 0.5 μM, 0.6 μM, 0.7 μM, 0.8 μM, 0.9 μM, or 1.0 μM, in the perilymph fluid in the inner ear and VPA is administered in amount sufficient to achieve a concentration of about is about 100 μM to 4 mM in the perilymph fluid in the inner ear. Alternatively, the EPT-103182 is administered to a subject, for example to the middle ear at a concentration of about 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, or about 1 mM; the GSK3-inhibitor XXII is administered, in amount sufficient to achieve a concentration of about 0.1 mM, 0.2 mM, 0.3 mM, 0.4 mM, 0.5 mM, 0.6 mM, 0.7 mM, 0.8 mM, 0.9 mM, or 1.0 mM, in the perilymph fluid in the inner ear and VPA to a subject, for example to the middle ear at a concentration about 100 mM to 4,000 mM.

In some embodiments the KDM inhibitor is EPT-103182; the Wnt agonist is CHIR99021 and the second epigenetic agent is VPA. In some embodiments, the EPT-103182 is administered, in amount sufficient to achieve a concentration of about 0.1 nM, 0.2 nM, 0.3 nM, 0.4 nM, 0.5 nM, 0.6 nM, 0.7 nM, 0.8 nM, 0.9 nM, 1.0 nM, 2.0 nM, 3.0 nM, 4.0 nM, 5.0 nM, 6.0 nM, 7.0 nM, 8.0 nM, 9.0 nM, 10 nM, 20 nM, 30 nM, 40 nM, 50 nM, 60 nM, 70 nM, 80 nM, 90 nM, 100 nM, 200 nM, 300 nM, 400 nM, 500 nM, 600 nM, 700 nM, 800 nM, 900 nM, or about 1 μM in the perilymph fluid in the inner ear; CHIR99021 is administered, in amount sufficient to achieve a concentration of about 1 μM, 2 μM, 3 μM, 4 μM, 5 μM, 6 μM, 7 μM, 8 μM, 9 μM, or 10 μM, in the perilymph fluid in the inner ear and VPA is administered in amount sufficient to achieve a concentration of about is about 100 μM to 4 mM in the perilymph fluid in the inner ear. Alternatively, the EPT-103182 is administered to a subject, for example to the middle ear at a concentration of about 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, or about 1 mM; CHIR99021 is administered to a subject, for example to the middle ear at a concentration of about 1 mM, 2 mM, 3 mM, 4 mM, 5 mM, 6 mM, 7 mM, 8 mM, 9 mM, or 10 mM and VPA to a subject, for example to the middle ear at a concentration about 100 mM to 4,000 mM.

Some embodiments comprise administering the (i) epigenetic agent and (ii) Wnt agonist together in the same pharmaceutical composition, as described herein. Some embodiments comprise administering the (i) epigenetic agent and (ii) Wnt agonist separately in separate pharmaceutical compositions.

Some embodiments comprise administering the (i) epigenetic agent, (ii) Wnt agonist, and (iii) the additional epigenetic agent(s) together in the same pharmaceutical composition, as described herein. Some embodiments comprise administering the (i) epigenetic agent (ii) Wnt agonist and (iii) the additional epigenetic agent(s) Wnt agonist separately in separate pharmaceutical compositions.

Some embodiments comprise administering the (i) epigenetic agent, (ii) Wnt agonist, and (iii) the additional epigenetic agent(s) together in the same pharmaceutical composition, as described herein and the (iii) epigenetic agent in a pharmaceutical composition.

### Pharmaceutical Compositions and Administration

Certain embodiments relate to pharmaceutical, prophylactic, and/or therapeutic compositions, comprising a pharmaceutically-acceptable carrier and an epigenetic agent and a Wnt agonist (and optionally a second epigenetic agent) a pharmaceutically-acceptable salt thereof or combinations thereof as described herein (collectively referred to herein as the “compound(s)”).

Certain embodiments relate to pharmaceutical, prophylactic, and/or therapeutic compositions, comprising a pharmaceutically-acceptable carrier and an epigenetic agent and a Wnt agonist (and optionally a second epigenetic agent) a pharmaceutically-acceptable salt thereof or combinations thereof as described herein (collectively referred to herein as the “compound(s)”).

In some embodiments, the concentration of the compound(s) in the pharmaceutical compositions of the invention are at the “formulation effective concentration” as described supra.

In some embodiments, the pharmaceutical composition comprises a epigenetic agent at a concentration of about 0.01 nM to 1000 μM, about 1 nM to 100 μM, about 10 nM to 10 μM, about 1 nM to 10 μM, about 10 μM to 100 μM, about 100 μM to 1000 μM, about 1 μM to 10 μM, 0.01 mM to 1000 mM, about 1 mM to 100 mM, or about 10 mM to 100 mM.

In some embodiments, the pharmaceutical composition comprises a LSD1 inhibitor that is GSK-2879552 at a concentration of about 0.001 μM to 1,000 mM, about 0.01 μM to 100,000 μM, about 0.1 μM to 10,000 μM, about 1 μM to 1,000 μM, about 1 μM to 10 μM, about 10 μM to 100 μM, about 100 μM to 1 mM, or about 1 mM to 10 mM.

In some embodiments, the pharmaceutical composition comprises a LSD1 inhibitor that is GSK-2879552 at a concentration of about 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, 10 mM, 12 mM, 14 mM, 16 mM, 18 mM, 20 mM, 25 mM, or about 30 mM.

In some embodiments, the pharmaceutical composition comprises GSK-2879552 at a unit dose of about 0.01 mg to 500 mg about 0.1 mg to 100 mg, about 1 mg to 50 mg, about 1 mg to 25 mg, about 1 mg to 10 mg, about 1 mg to 5 mg, about 0.01 mg to 0.1 mg, about 0.1 mg to 1 mg, about 1 mg to 10 mg, about 10 mg to 100 mg, about 100 mg to 500 mg, about 0.5 mg to 1 mg, about 1 mg to 2 mg, about 2 mg to 3 mg, about 3 mg to 4 mg, about 4 mg to 5 mg, or about 5-10 mg.

In some embodiments, the pharmaceutical composition comprises a LSD1 inhibitor that is GSK-LSD1 at a concentration of about 0.001 μM to 10 mM, about 0.01 μM to 1 mM, about 0.1 μM to 100 μM, about 0.001 μM to 0.01 μM, about 0.01 μM to 0.1 μM, about 0.1 μM to 1 μM, about 1 μM to 10 μM, about 10 μM to 100 μM, or about 100 μM to 1,000 μM.

In some embodiments, the pharmaceutical composition comprises a LSD1 inhibitor that is GSK-LSD1 at a concentration of about 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 1 mM, 5 mM, 10 mM, or 50 mM.

In some embodiments, the pharmaceutical composition comprises GSK-LSD1 at a unit dose of about of about 0.01 mg to 500 mg, about 0.1 mg to 100 mg, about 1 mg to 50 mg, about 1 mg to 25 mg, about 1 mg to 10 mg, about 1 mg to 5 mg, about 0.01 mg to 0.1 mg, about 0.1 mg to 1 mg, about 1 mg to 10 mg, about 10 mg to 100 mg, about 100 mg to 500 mg, about 0.5 mg to 1 mg, about 1 mg to 2 mg, about 2 mg to 3 mg, about 3 mg to 4 mg, about 4 mg to 5 mg, about 5-10 mg, about 10-25 mg, about 25-50 mg, or about 50-100 mg.

In some embodiments, the pharmaceutical composition comprises a LSD1 inhibitor that is Tranylcypromine at a concentration of about 0.001 mM to 10,000 mM, about 0.01 mM to 1,000 mM, about 0.1 mM to 100 mM, about 0.001 mM to 0.01 mM, about 0.01 mM to 0.1 mM, about 0.1 mM to 1 mM, about 1 mM to 10 mM, about 10 mM to 100 mM, about 100 mM to 1,000 mM, or about 1,000 mM to 10,000 mM.

In some embodiments, the pharmaceutical composition comprises a LSD1 inhibitor that is Tranylcypromine at a concentration of about 0.1 mM, 0.2 mM, 0.3 mM, 0.4 mM, 0.5 mM, 0.6 mM, 0.7 mM, 0.8 mM, 0.9 mM, 1 mM, 2 mM, 3 mM, 4 mM, 5 mM, 6 mM, 7 mM, 8 mM, 9 mM, 10 mM, 12 mM, 14 mM, 16 mM, 18 mM, or 20 mM.

In some embodiments, the pharmaceutical composition comprises Tranylcypromine at a unit dose of about 1.5 mg to 750 mg, about 5 mg to 500 mg, about 10 mg to 250 mg, about 15 mg to 150 mg, about 1.5 mg to 10 mg, about 10 mg to 20 mg, about 20 mg to 30 mg, about 30 mg to 40 mg, about 40 mg to 50 mg, about 50 mg to 60 mg, about 60 mg to 70 mg, about 70 mg to 80 mg, about 90 mg to 100 mg, about 100 mg to 120 mg, or about 120 mg to 150 mg.

In some embodiments, the pharmaceutical composition comprises a LSD1 inhibitor that is Phenelzine sulfate at a concentration of about 0.001 mM to 100,000 mM, 0.01 mM to 10,000 mM, about 0.1 mM to 1,000 mM, about 0.1 mM to 100 mM, about 0.001 mM to 0.01 mM, about 0.01 mM to 0.1 mM, about 0.1 mM to 1 mM, about 1 mM to 10 mM, about 10 mM to 100 mM, about 100 mM to 1,000 mM, or about 1,000 mM to 10,000 mM.

In some embodiments, the pharmaceutical composition comprises a LSD1 inhibitor that is Phenelzine sulfate at a concentration of about 0.1 mM, 0.2 mM, 0.3 mM, 0.4 mM, 0.5 mM, 0.6 mM, 0.7 mM, 0.8 mM, 0.9 mM, 1 mM, 2 mM, 3 mM, 4 mM, 5 mM, 6 mM, 7 mM, 8 mM, 9 mM, or 10 mM.

In some embodiments, the pharmaceutical composition comprises Phenelzine sulfate at a unit dose of about 1.5 mg to 750 mg, about 5 mg to 500 mg, about 10 mg to 250 mg, about 15 mg to 150 mg, about 1.5 mg to 10 mg, about 10 mg to 20 mg, about 20 mg to 30 mg; about 30 mg to 40 mg; about 40 mg to 50 mg about 50 mg to 60 mg; about 60 mg to 70 mg; about 70 mg to 80 mg; or about 90 mg to 100 mg.

In some embodiments, the pharmaceutical composition comprises a LSD1 inhibitor that is ORY-1001 at a concentration of about 0.001 mM to 100,000 mM, 0.01 mM to 10,000 mM, about 0.1 mM to 1,000 mM, about 0.1 mM to 100 mM, about 0.001 mM to 0.01 mM, about 0.01 mM to 0.1 mM, about 0.1 mM to 1 mM, about 1 mM to 10 mM, about 10 mM to 100 mM, about 100 mM to 1,000 mM, or about 1,000 mM to 10,000 mM.

In some embodiments, the pharmaceutical composition comprises a LSD1 inhibitor that is ORY-1001 at a concentration of about 10 nM, 20 nM, 30 nM, 40 nM, 50 nM, 60 nM, 70 nM, 80 nM, 90 nM, 100 nM, 0.2 μM, 0.3 μM, 0.4 μM, 0.5 μM, 0.6 μM, 0.7 μM, 0.8 μM, 0.9 μM, 1 μM, 2 μM, 3 μM, 4 μM, 5 μM, 6 μM, 7 μM, 8 μM, 9 μM, 10 μM, 10 μM, 20 μM, 30 μM, 40 μM, 50 μM, 60 μM, 70 μM, 80 μM, 90 μM, 0.1 mM, 0.2 mM, 0.3 mM, 0.4 mM, 0.5 mM, 0.6 mM, 0.7 mM, 0.8 mM, 0.9 mM, 1 mM, 2 mM, 3 mM, 4 mM, 5 mM, 6 mM, 7 mM, 8 mM, 9 mM, or 10 mM.

In some embodiments, the pharmaceutical composition comprises ORY-1001 at a unit dose of about 1.5 mg to 750 mg, about 5 mg to 500 mg, about 10 mg to 250 mg, about 15 mg to 150 mg, about 1.5 mg to 10 mg, about 10 mg to 20 mg, about 20 mg to 30 mg; about 30 mg to 40 mg; about 40 mg to 50 mg about 50 mg to 60 mg; about 60 mg to 70 mg; about 70 mg to 80 mg; or about 90 mg to 100 mg.

In some embodiments, the pharmaceutical composition comprises a LSD1 inhibitor that is RN-1 at a concentration of about 0.001 mM to 100,000 mM, 0.01 mM to 10,000 mM, about 0.1 mM to 1,000 mM, about 0.1 mM to 100 mM, about 0.001 mM to 0.01 mM, about 0.01 mM to 0.1 mM, about 0.1 mM to 1 mM, about 1 mM to 10 mM, about 10 mM to 100 mM, about 100 mM to 1,000 mM, or about 1,000 mM to 10,000 mM.

In some embodiments, the pharmaceutical composition comprises a LSD1 inhibitor that is RN-1 at a concentration of about 1 μM, 2 μM, 3 μM, 4 μM, 5 μM, 6 μM, 7 μM, 8 μM, 9 μM, 10 μM, 10 μM, 20 μM, 30 μM, 40 μM, 50 μM, 60 μM, 70 μM, 80 μM, 90 μM, 0.1 mM, 0.2 mM, 0.3 mM, 0.4 mM, 0.5 mM, 0.6 mM, 0.7 mM, 0.8 mM, 0.9 mM, 1 mM, 2 mM, 3 mM, 4 mM, 5 mM, 6 mM, 7 mM, 8 mM, 9 mM, or 10 mM.

In some embodiments, the pharmaceutical composition comprises RN-1 at a unit dose of about 1.5 mg to 750 mg, about 5 mg to 500 mg, about 10 mg to 250 mg, about 15 mg to 150 mg, about 1.5 mg to 10 mg, about 10 mg to 20 mg, about 20 mg to 30 mg; about 30 mg to 40 mg; about 40 mg to 50 mg about 50 mg to 60 mg; about 60 mg to 70 mg; about 70 mg to 80 mg; or about 90 mg to 100 mg.

In some embodiments, the pharmaceutical composition comprises a GSK3 Inhibitor that is AZD1080, at a concentration of about 0.001 mM to 10,000 mM, about 0.01 mM to 1,000 mM, about 0.1 mM to 100 mM, about 0.001 mM to 0.01 mM, about 0.01 mM to 0.1 mM, about 0.1 mM to 1 mM, about 1 mM to 10 mM, about 10 mM to 100 mM, about 100 mM to 1,000 mM, or about 1,000 mM to 10,000 nM. In some embodiments, the AZD1080 is at a concentration of about 1 mM, 2 mM, 3 mM, 4 mM, 5 mM, 6 mM, 7 mM, 8 mM, 9 mM, or 10 mM.

In some embodiments, the pharmaceutical composition comprises a GSK3 Inhibitor that is LY2090314 at a concentration of about 0.001 μM to 10 mM, about 0.01 μM to 1 mM, about 0.1 μM to 100 uM, about 0.001 μM to 0.01 μM, about 0.01 μM to 0.1 μM, about 0.1 μM to 1 μM, about 1 μM to 10 μM, about 10 μM to 100 μM, about 100 μM to 1 mM, or about 1 mM to 10 mM. In some embodiments, LY2090314 is at a concentration of about 1 μM, 5 μM, 10 μM, 15 μM, 20 μM, or 40 μM.

In some embodiments, the pharmaceutical composition comprises a GSK3 Inhibitor that is a substituted 3-Imidazo[1,2-a]pyridin-3-yl-4-(1,2,3,4-tetrahydro-[1,4]diazepino-[6,7,1-hi]indol-7-yl)pyrrole-2,5-dione at a concentration of about 0.001 μM to 10 mM, about 0.01 μM to 1 mM, about 0.1 μM to 100 uM, about 0.001 μM to 0.01 μM, about 0.01 μM to 0.1 μM, about 0.1 μM to 1 μM, about 1 μM to 10 μM, about 10 μM to 100 μM, about 100 μM to 1 mM, or about 1 mM to 10 mM. In some embodiments, the substituted 3-Imidazo[1,2-a]pyridin-3-yl-4-(1,2,3,4-tetrahydro-[1,4]diazepino-[6,7,1-hi]indol-7-yl)pyrrole-2,5-dione, is at a concentration of about 1 μM, 5 μM, 10 μM, 15 μM, 20 μM, 50 μM, 100 μM, 250 μM, or 500 μM.

In some embodiments, the pharmaceutical composition comprises a GSK3 Inhibitor that is GSK3-inhibitor XXII, at a concentration of about 0.1 μM to 1,000 mM, about 1 μM to 100 mM, about 10 μM to 10 mM, about 0.1 μM to 1 μM, about 1 μM to 10 μM, about 10 μM to 100 μM, about 100 μM to 1 mM, about 1 mM to 10 mM, about 10 mM to 100 mM, or about 100 mM to 1000 mM. In some embodiments, the GSK3-inhibitor XXII is at a concentration of about 0.1 mM, 0.2 mM, 0.3 mM, 0.4 mM, 0.5 mM, 0.6 mM, 0.7 mM, 0.8 mM, 0.9 mM, or 1.0 mM.

In some embodiments, the pharmaceutical composition comprises a GSK3 Inhibitor that is CHIR99021 at a concentration of about 0.001 mM to 10,000 mM, about 0.01 mM to 1,000 mM, about 0.1 mM to 100 mM, about 0.001 mM to 0.01 mM, about 0.01 mM to 0.1 mM, about 0.1 mM to 1 mM, about 1 mM to 10 mM, about 10 mM to 100 mM, about 100 mM to 1,000 mM, or about 1,000 mM to 10,000 mM. In some embodiments, the CHIR99021 is at a concentration of about 1 mM, 2 mM, 3 mM, 4 mM, 5 mM, 6 mM, 7 mM, 8 mM, 9 mM, or 10 mM.

In some embodiments, the pharmaceutical composition comprises an epigenetic agent that is an HDAC inhibitor at a concentration about 10 uM to 1,000,000 mM, about 1000 uM to 100,000 mM, about 10,000 uM to 10,000 mM, about 1000 uM to 10,000 uM, about 10,000 uM to 100,000 uM, about 100,000 uM to 1,000,000 uM, about 1,000 mM to 10,000 mM, or about 10,000 mM to 100,000 mM.

In some embodiments, the pharmaceutical composition comprises a HDAC inhibitor that is VPA at a concentration about 100 mM to 4,000 mM.

In some embodiments, the pharmaceutical composition comprises VPA at a unit dose of about 50 mg, about 100 mg, about 125 mg, about 250 mg, about 500 mg, 1000 mg, 2000 mg, 3000 mg, 4000 mg, or about 5000 mg

In some embodiments, the pharmaceutical composition comprises an oral dosage form of VPA at a unit dose of about 50 mg, about 100 mg, about 125 mg, about 250 mg, about 500 mg, 1000 mg, 2000 mg, 3000 mg, 4000 mg, or about 5000 mg

In some embodiments, the pharmaceutical composition comprises a HDAC inhibitor that is is 2-hexyl-4-pentynoic acid at concentration about 100 mM to 4,000 mM.

In some embodiments, the pharmaceutical composition comprises 2-hexyl-4-pentynoic acid at a unit dose of 50 mg, about 100 mg, about 125 mg, about 250 mg, about 500 mg, 1000 mg, 2000 mg, 3000 mg, 4000 mg, or about 5000 mg

In some embodiments, the pharmaceutical composition comprises an oral dosage form of 2-hexyl-4-pentynoic acid at a unit dose of about 50 mg, about 100 mg, about 125 mg, about 250 mg, about 500 mg, 1000 mg, 2000 mg, 3000 mg, 4000 mg, or about 5000 mg

In some embodiments, the pharmaceutical composition comprises, Na phenylbutyrate that is at a concentration about 100 mM to 4,000 mM.

In some embodiments, the pharmaceutical composition comprises Na phenylbutyrate at a unit dose of about 50 mg, about 100 mg, about 125 mg, about 250 mg, about 500 mg, 1000 mg, 2000 mg, 3000 mg, 4000 mg, or about 5000 mg

In some embodiments, the pharmaceutical composition comprises an oral dosage form of the Na phenylbutyrate at a unit dose of about 50 mg, about 100 mg, about 125 mg, about 250 mg, about 500 mg, 1000 mg, 2000 mg, 3000 mg, 4000 mg, or about 5000 mg

In some embodiments, the pharmaceutical composition comprises an epigenetic agent that is an EZH2 inhibitor

In some embodiments, the pharmaceutical composition comprises an EZH2 inhibitor that is PF-06821497 at a concentration of 0.001 μM to 100 mM, about 0.01 μM to 10 mM, about 0.1 μM to 1 mM, about 1 μM to 100 μM, about 1 μM to 10 μM, 10 μM to 100 μM, or about 100 μM to 1 mM.

In some embodiments, the pharmaceutical composition comprises an EZH2 inhibitor that is PF-06821497 at at a concentration of about 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, or about 1 mM.

In some embodiments, the pharmaceutical composition comprises PF-06821497 at a daily dose of about 50 mg to 5,000 mg, about 50 mg to 4000 mg, about 50 mg to 3000 mg, about 50 mg to 2000 mg, about 50 mg to 1000 mg, about 50 mg to 500 mg, about 100 mg to 2500 mg, about 100 mg to 2000 mg, about 100 mg to 1500 mg, about 100 mg to 1000 mg, about 100 mg to 500 mg, about 150 mg to 2500 mg, about 150 mg to 2000 mg, about 150 mg to 1500 mg, about 150 mg to 1250 mg, about 75 mg, about 100 mg, about 150 mg, about 200 mg, about 300 mg, about 400 mg, about 500 mg, about 600 mg, about 700 mg, about 800 mg, about 900 mg, about 1000 mg, about 1200 mg, about 1400 mg, about 1600 mg, about 1800 mg, or about 2000 mg.

In some embodiments, the pharmaceutical composition comprises an EZH2 inhibitor that is CPI-1205 at a concentration of 0.001 μM to 100 mM, about 0.01 μM to 10 mM, about 0.1 μM to 1 mM, about 1 μM to 100 μM, about 1 μM to 10 μM, about 10 μM to 100 μM, or about 100 μM to 1000 μM.

In some embodiments, the pharmaceutical composition comprises CPI-1205 is that is at a concentration of about 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, or about 1 mM.

In some embodiments, the pharmaceutical composition comprises an EZH2 inhibitor that is CPI-1205 ay a unit dose about 100 to 5,000 mg, about 100 mg to 4000 mg, about 100 mg to 3000 mg, about 100 mg to 2000 mg, about 500 to 5,000 mg, about 500 mg to 4000 mg, about 500 mg to 3000 mg, about 750 to 5,000 mg, about 750 mg to 4000 mg, about 750 mg to 3000 mg, about 800 mg to 2400 mg, about 400 mg, about 600 mg, about 800 mg, about 1000 mg, about 1200 mg, about 1400 mg, about 1600 mg, about 1800 mg, about 2000 mg, about 2200 mg, about 2400 mg, about 2600 mg, about 2800 mg, about 3000 mg, about 3250 mg, about 3500 mg, about 4000 mg, about 4500 mg, or about 5000 mg.

In some embodiments, the pharmaceutical composition comprises an EZH2 inhibitor that is valemetostat at a concentration of about 0.001 μM to 100 mM, about 0.01 μM to 10 mM, about 0.1 μM to 1 mM, about 1 μM to 100 μM, about 1 μM to 10 μM, 10 μM to 100 μM, or about 100 μM to 1000 μM.

In some embodiments, the pharmaceutical composition comprises Valemetostat that is at a concentration of about 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, or 1 mM.

In some embodiments, the pharmaceutical composition comprises an EZH2 inhibitor is valemetostat at a unit dose of about 50 mg to 5,000 mg, about 50 mg to 4000 mg, about 50 mg to 3000 mg, about 50 mg to 2000 mg, about 50 mg to 1000 mg, about 50 mg to 500 mg, about 100 mg to 2000 mg, about 100 mg to 1500 mg, about 100 mg to 1000 mg, about 100 mg to 500 mg, about 100 mg, about 200 mg, about 300 mg, about 400 mg, about 500 mg, about 600 mg, about 700 mg, about 800 mg, about 900 mg, about 1000 mg, about 1200 mg, about 1400 mg, about 1600 mg, about 1800 mg, or about 2000 mg.

In some embodiments, the pharmaceutical composition comprises an EZH2 inhibitor that is tazemetostat at a concentration of about 0.001 μM to 100 mM, about 0.01 μM to 10 mM, about 0.1 μM to 1 mM, about 1 μM to 100 μM, about 1 μM to 10 μM, 10 μM to 100 μM, about 100 μM to 1000 μM or about 1 mM to 10 mM.

In some embodiments, the pharmaceutical composition comprises tazemetostat at a concentration of about 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, or 10 mM.

In some embodiments, the pharmaceutical composition comprises an EZH2 inhibitor that is tazemetostat at a unit dose of about 50 mg to 5,000 mg, about 50 mg to 4000 mg, about 50 mg to 3000 mg, about 50 mg to 2000 mg, about 50 mg to 1000 mg, about 50 mg to 500 mg, about 100 mg to 2500 mg, about 100 mg to 2000 mg, about 100 mg to 1500 mg, about 100 mg to 1000 mg, about 100 mg to 500 mg, about 200 mg to 2500 mg, about 200 mg to 2000 mg, about 200 mg to 1600 mg, about 200 mg to 1000 mg, about 100 mg, about 200 mg, about 300 mg, about 400 mg, about 500 mg, about 600 mg, about 700 mg, about 800 mg, about 900 mg, about 1000 mg, about 1200 mg, about 1400 mg, about 1600 mg, about 1800 mg, or about 2000 mg.

In some embodiments, the pharmaceutical composition comprises an EZH2 inhibitor that is E11 at a concentration of about 0.1 μM to 1000 mM, about 1 μM to 100 mM, about 10 μM to 10 mM, about 100 μM to 10 mM, about 1 μM to 10 μM, 10 μM to 100 μM, about 100 μM to 1000 μM, 1 mM to 10 mM, or about 10 mM to 100 mM.

In some embodiments, the pharmaceutical composition comprises E11 at a concentration of about 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, 10 mM, 15 mM, 20 mM, 25 mM, 30 mM, 35 mM, 40 mM, 45 mM, or about 50 mM.

In some embodiments, the pharmaceutical composition comprises an EZH2 inhibitor is E11 at a unit dose of about 50 mg to 5,000 mg, about 50 mg to 4000 mg, about 50 mg to 3000 mg, about 50 mg to 2000 mg, about 50 mg to 1000 mg, about 50 mg to 500 mg, about 100 mg to 2500 mg, about 100 mg to 2000 mg, about 100 mg to 1500 mg, about 100 mg to 1000 mg, about 100 mg to 500 mg, about 200 mg to 2500 mg, about 200 mg to 2000 mg, about 200 mg to 1500 mg, about 200 mg to 1000 mg, about 100 mg, about 200 mg, about 300 mg, about 400 mg, about 500 mg, about 600 mg, about 700 mg, about 800 mg, about 900 mg, about 1000 mg, about 1200 mg, about 1400 mg, about 1600 mg, about 1800 mg, or about 2000 mg.

In some embodiments, the pharmaceutical composition comprises an EZH2 inhibitor that is CPI-169 at a concentration of about 0.1 μM to 1000 mM, about 1 μM to 100 mM, about 10 μM to 10 mM, about 100 μM to 10 mM, about 1 μM to 10 μM, 10 μM to 100 μM, about 100 μM to 1000 μM, 1 mM to 10 mM, or about 10 mM to 100 mM.

In some embodiments, the pharmaceutical composition comprises CPI-169 at a concentration of about 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, 10 mM, 15 mM, 20 mM, 25 mM, 30 mM, 35 mM, 40 mM, 45 mM, or about 50 mM.

In some embodiments, the pharmaceutical composition comprises an EZH2 inhibitor that is CPI-169 at a unit dose of about 50 mg to 5,000 mg/day, about 50 mg to 4000 mg/day, about 50 mg to 3000 mg/day, about 50 mg to 2000 mg/day, about 50 mg to 1000 mg/day, about 50 mg to 500 mg/day, about 100 mg to 2500 mg/day, about 100 mg to 2000 mg/day, about 100 mg to 1500 mg/day, about 100 mg to 1000 mg/day, about 100 mg to 500 mg/day, about 200 mg to 2500 mg/day, about 200 mg to 2000 mg/day, about 200 mg to 1500 mg/day, about 200 mg to 1000 mg/day, about 100 mg/day, about 200 mg/day, about 300 mg/day, about 400 mg/day, about 500 mg/day, about 600 mg/day, about 700 mg/day, about 800 mg/day, about 900 mg/day, about 1000 mg/day, about 1200 mg/day, about 1400 mg/day, about 1600 mg/day, about 1800 mg/day, or about 2000 mg/day.

In some embodiments, the pharmaceutical composition comprises an EZH2 inhibitor that is CPI-360 at a concentration of about 0.1 μM to 1000 mM, about 1 μM to 100 mM, about 10 μM to 10 mM, about 100 μM to 10 mM, about 1 μM to 10 μM, 10 μM to 100 μM, about 100 μM to 1000 μM, 1 mM to 10 mM, or about 10 mM to 100 mM.

In some embodiments, the pharmaceutical composition comprises CPI-360 at a concentration of about 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, 10 mM, 15 mM, 20 mM, 25 mM, 30 mM, 35 mM, 40 mM, 45 mM, or about 50 mM.

In some embodiments, the pharmaceutical composition comprises an EZH2 inhibitor that is CPI-360 at a unit dose of about 50 mg to 5,000 mg/day, about 50 mg to 4000 mg/day, about 50 mg to 3000 mg/day, about 50 mg to 2000 mg/day, about 50 mg to 1000 mg/day, about 50 mg to 500 mg/day, about 100 mg to 2500 mg/day, about 100 mg to 2000 mg/day, about 100 mg to 1500 mg/day, about 100 mg to 1000 mg/day, about 100 mg to 500 mg/day, about 200 mg to 2500 mg/day, about 200 mg to 2000 mg/day, about 200 mg to 1500 mg/day, about 200 mg to 1000 mg/day, about 100 mg/day, about 200 mg/day, about 300 mg/day, about 400 mg/day, about 500 mg/day, about 600 mg/day, about 700 mg/day, about 800 mg/day, about 900 mg/day, about 1000 mg/day, about 1200 mg/day, about 1400 mg/day, about 1600 mg/day, about 1800 mg/day, or about 2000 mg/day.

In some embodiments, the pharmaceutical composition comprises an EZH2 inhibitor that is EPZ011989 at a concentration of about 0.1 μM to 1000 mM, about 1 μM to 100 mM, about 10 μM to 10 mM, about 100 μM to 10 mM, about 1 μM to 10 μM, 10 μM to 100 μM, about 100 μM to 1000 μM, 1 mM to 10 mM, or about 10 mM to 100 mM.

In some embodiments, the pharmaceutical composition comprises EPZ011989 at a concentration of about 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, 10 mM, 15 mM, 20 mM, 25 mM, 30 mM, 35 mM, 40 mM, 45 mM, or about 50 mM.

In some embodiments, the pharmaceutical composition comprises an EZH2 inhibitor that is EPZ011989 at a unit dose of about 50 mg to 5,000 mg/day, about 50 mg to 4000 mg/day, about 50 mg to 3000 mg/day, about 50 mg to 2000 mg/day, about 50 mg to 1000 mg/day, about 50 mg to 500 mg/day, about 100 mg to 2500 mg/day, about 100 mg to 2000 mg/day, about 100 mg to 1500 mg/day, about 100 mg to 1000 mg/day, about 100 mg to 500 mg/day, about 200 mg to 2500 mg/day, about 200 mg to 2000 mg/day, about 200 mg to 1500 mg/day, about 200 mg to 1000 mg/day, about 100 mg/day, about 200 mg/day, about 300 mg/day, about 400 mg/day, about 500 mg/day, about 600 mg/day, about 700 mg/day, about 800 mg/day, about 900 mg/day, about 1000 mg/day, about 1200 mg/day, about 1400 mg/day, about 1600 mg/day, about 1800 mg/day, or about 2000 mg/day.

In some embodiments, the pharmaceutical composition comprises an EZH2 inhibitor that is UNC 2399 at a concentration of about 0.1 μM to 1000 mM, about 1 μM to 100 mM, about 10 μM to 10 mM, about 100 μM to 10 mM, about 1 μM to 10 μM, 10 μM to 100 μM, about 100 μM to 1000 μM, 1 mM to 10 mM, or about 10 mM to 100 mM.

In some embodiments, the pharmaceutical composition comprises UNC 2399 at a concentration of about 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, 10 mM, 15 mM, 20 mM, 25 mM, 30 mM, 35 mM, 40 mM, 45 mM, or about 50 mM.

In some embodiments, the pharmaceutical composition comprises an EZH2 inhibitor that is UNC 2399 at a unit dose of about 50 mg to 5,000 mg/day, about 50 mg to 4000 mg/day, about 50 mg to 3000 mg/day, about 50 mg to 2000 mg/day, about 50 mg to 1000 mg/day, about 50 mg to 500 mg/day, about 100 mg to 2500 mg/day, about 100 mg to 2000 mg/day, about 100 mg to 1500 mg/day, about 100 mg to 1000 mg/day, about 100 mg to 500 mg/day, about 200 mg to 2500 mg/day, about 200 mg to 2000 mg/day, about 200 mg to 1500 mg/day, about 200 mg to 1000 mg/day, about 100 mg/day, about 200 mg/day, about 300 mg/day, about 400 mg/day, about 500 mg/day, about 600 mg/day, about 700 mg/day, about 800 mg/day, about 900 mg/day, about 1000 mg/day, about 1200 mg/day, about 1400 mg/day, about 1600 mg/day, about 1800 mg/day, or about 2000 mg/day.

In some embodiments, the pharmaceutical composition comprises an EZH2 inhibitor that is PF-06726304 at a concentration of about 0.001 μM to 100 mM, about 0.01 μM to 10 mM, about 0.1 μM to 1 mM, about 1 μM to 100 μM, about 1 μM to 10 μM, 10 μM to 100 μM, about 100 μM to 1000 μM or about 1 mM to 10 mM.

In some embodiments, the pharmaceutical composition comprises PF-06726304 at a concentration of about 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, or 10 mM.

In some embodiments, the pharmaceutical composition comprises an EZH2 inhibitor that is PF-06726304 at a unit dose of about 50 mg to 5,000 mg, about 50 mg to 4000 mg, about 50 mg to 3000 mg, about 50 mg to 2000 mg, about 50 mg to 1000 mg, about 50 mg to 500 mg, about 100 mg to 2500 mg, about 100 mg to 2000 mg, about 100 mg to 1500 mg, about 100 mg to 1000 mg, about 100 mg to 500 mg, about 200 mg to 2500 mg, about 200 mg to 2000 mg, about 200 mg to 1600 mg, about 200 mg to 1000 mg, about 100 mg, about 200 mg, about 300 mg, about 400 mg, about 500 mg, about 600 mg, about 700 mg, about 800 mg, about 900 mg, about 1000 mg, about 1200 mg, about 1400 mg, about 1600 mg, about 1800 mg, or about 2000 mg.

In some embodiments the additional epigenetic agent is a DOTL1 inhibitor.

In some embodiments, the pharmaceutical composition comprises a DOT1L inhibitor that is EPZ004777 at a unit dose of about 1-1000 mg , about 10-100 mg, about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 10 mg to 5,000 mg, about 10 mg to 3000 mg, about 10 mg to 1000 mg, about 10 mg to 500 mg, 20 mg to 5,000 mg, about 20 mg to 1000 mg, about 20 mg to 500 mg, about 10 mg, about 25 mg, about 50 mg, about 75 mg, about 100 mg, about 150 mg, about 200 mg, about 300 mg, about 400 mg, about 500 mg, about 600 mg, about 700 mg, about 800 mg, about 900 mg, or about 1000 mg.

In some embodiments, the pharmaceutical composition comprises EPZ004777 at a concentration of about 0.01 μM to 1000 mM, about 0.1 μM to 100 mM, about 1 μM to 10 mM, about 10 μM to 1 mM, 10 μM to 100 μM, about 100 μM to 1000 μM, about 1 mM to 10 mM, or about 10 mM to 100 mM.

In some embodiments, the pharmaceutical composition comprises EPZ004777 at a concentration of about 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, 10 mM, 11 mM, 12 mM, 13 mM, 14 mM, 15 mM, 20 mM, 25 mM, 30 mM, 35 mM, 40 mM, 45 mM, or about 50 mM.

In some embodiments, the pharmaceutical composition comprises a DOT1L inhibitor is EPZ004777 at a unit dose of about 1-1000 mg, about 10-100 mg, about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 10 mg to 5,000 mg, about 10 mg to 3000 mg, about 10 mg to 1000 mg, about 10 mg to 500 mg, 20 mg to 5,000 mg, about 20 mg to 1000 mg, about 20 mg to 500 mg, about 10 mg, about 25 mg, about 50 mg, about 75 mg, about 100 mg, about 150 mg, about 200 mg, about 300 mg, about 400 mg, about 500 mg, about 600 mg, about 700 mg, about 800 mg, about 900 mg, or about 1000 mg.

In some embodiments, the pharmaceutical composition comprises a DOT1L inhibitor is EPZ004777 formulated for IV administration at a unit dose of 1-1000 mg, about 10-100 mg, about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 10 mg to 5,000 mg, about 10 mg to 3000 mg, about 10 mg to 1000 mg, about 10 mg to 500 mg, 20 mg to 5,000 mg, about 20 mg to 1000 mg, about 20 mg to 500 mg, about 10 mg, about 25 mg, about 50 mg, about 75 mg, about 100 mg, about 150 mg, about 200 mg, about 300 mg, about 400 mg, about 500 mg, about 600 mg, about 700 mg, about 800 mg, about 900 mg, or about 1000 mg.

In some embodiments, the pharmaceutical composition comprises a DOT1L inhibitor that is SGC0946 at a concentration of about 0.01 μM to 1000 mM, about 0.1 μM to 100 mM, about 1 μM to 10 mM, about 10 μM to 1 mM, 10 μM to 100 μM, about 100 μM to 1000 μM, about 1 mM to 10 mM, or about 10 mM to 100 mM.

In some embodiments, the pharmaceutical composition comprises SGC0946 that is at a concentration of about 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, 10 mM, 11 mM, 12 mM, 13 mM, 14 mM, 15 mM, 20 mM, 25 mM, 30 mM, 35 mM, 40 mM, 45 mM, or about 50 mM.

In some embodiments, the pharmaceutical composition comprises a DOT1L inhibitor is SGC0946 at a unit dose of 1-1000 mg, about 10-100 mg, about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 10 mg to 5,000 mg, about 10 mg to 3000 mg, about 10 mg to 1000 mg, about 10 mg to 500 mg, 20 mg to 5,000 mg, about 20 mg to 1000 mg, about 20 mg to 500 mg, about 10 mg, about 25 mg, about 50 mg, about 75 mg, about 100 mg, about 150 mg, about 200 mg, about 300 mg, about 400 mg, about 500 mg, about 600 mg, about 700 mg, about 800 mg, about 900 mg, or about 1000 mg.

In some embodiments, the pharmaceutical composition comprises a DOT1 L inhibitor is SGC0946 formulated for IV administration at a unit dose of 1-1000 mg, about 10-100 mg, about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 10 mg to 5,000 mg, about 10 mg to 3000 mg, about 10 mg to 1000 mg, about 10 mg to 500 mg, 20 mg to 5,000 mg, about 20 mg to 1000 mg, about 20 mg to 500 mg, about 10 mg, about 25 mg, about 50 mg, about 75 mg, about 100 mg, about 150 mg, about 200 mg, about 300 mg, about 400 mg, about 500 mg, about 600 mg, about 700 mg, about 800 mg, about 900 mg, or about 1000 mg.

In some embodiments, the pharmaceutical composition comprises a DOT1L inhibitor that is pinometostat at a concentration of about 0.01 μM to 1000 mM, about 0.1 μM to 100 mM, about 1 μM to 10 mM, about 10 μM to 1 mM, 10 μM to 100 μM, about 100 μM to 1000 μM, about 1 mM to 10 mM, or about 10 mM to 100 mM.

In some embodiments, the pharmaceutical composition comprises a pinometostat a concentration of about 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, 10 mM, 11 mM, 12 mM, 13 mM, 14 mM, 15 mM, 20 mM, 25 mM, 30 mM, 35 mM, 40 mM, 45 mM, or about 50 mM.

In some embodiments, the pharmaceutical composition comprises a DOT1L inhibitor that is pinometostat at a unit dose of about 1-1000 mg, about 10-100 mg, about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 10 mg to 5,000 mg, about 10 mg to 3000 mg, about 10 mg to 1000 mg, about 10 mg to 500 mg, 20 mg to 5,000 mg, about 20 mg to 1000 mg, about 20 mg to 500 mg, about 10 mg, about 25 mg, about 50 mg, about 75 mg, about 100 mg, about 150 mg, about 200 mg, about 300 mg, about 400 mg, about 500 mg, about 600 mg, about 700 mg, about 800 mg, about 900 mg, or about 1000 mg.

In some embodiments, the pharmaceutical composition comprises a DOT1L inhibitor that is pinometostat formulated for IV administration at a unit dose of 1-1000 mg, about 10-100 mg, about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 10 mg to 5,000 mg, about 10 mg to 3000 mg, about 10 mg to 1000 mg, about 10 mg to 500 mg, 20 mg to 5,000 mg, about 20 mg to 1000 mg, about 20 mg to 500 mg, about 10 mg, about 25 mg, about 50 mg, about 75 mg, about 100 mg, about 150 mg, about 200 mg, about 300 mg, about 400 mg, about 500 mg, about 600 mg, about 700 mg, about 800 mg, about 900 mg, or about 1000 mg.

In some embodiments the additional epigenetic agent is a KDM inhibitor.

In some embodiments, the pharmaceutical composition comprises a KDM inhibitor that is AS 8351 at a concentration of about 0.01 μM to 1000 mM, about 0.1 μM to 100 mM, about 1 μM to 10 mM, about 10 μM to 1000 μM, about 1 μM to 10 μM, 10 μM to 100 μM, about 100 μM to 1000 μM or about 1 mM to 10 mM.

In some embodiments, the pharmaceutical composition comprises a AS 8351 at a concentration of about 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, or 10 mM.

In some embodiments, the pharmaceutical composition comprises a KDM inhibitor that is AS 8351 at a unit dose of about 50 mg to 5,000 mg, about 50 mg to 4000 mg, about 50 mg to 3000 mg, about 50 mg to 2000 mg, about 50 mg to 1000 mg, about 50 mg to 500 mg, about 100 mg to 2500 mg, about 100 mg to 2000 mg, about 100 mg to 1500 mg, about 100 mg to 1000 mg, about 100 mg to 500 mg, about 200 mg to 2500 mg, about 200 mg to 2000 mg, about 200 mg to 1600 mg, about 200 mg to 1000 mg, about 100 mg, about 200 mg, about 300 mg, about 400 mg, about 500 mg, about 600 mg, about 700 mg, about 800 mg, about 900 mg, about 1000 mg, about 1200 mg, about 1400 mg, about 1600 mg, about 1800 mg, or about 2000 mg.

In some embodiments, the pharmaceutical composition comprises a KDM inhibitor that is TC-E 5002 at a concentration of about 0.01 μM to 1000 mM, about 0.1 μM to 100 mM, about 1 μM to 10 mM, about 10 μM to 1000 μM, about 1 μM to 10 μM, 10 μM to 100 μM, about 100 μM to 1000 μM or about 1 mM to 10 mM.

In some embodiments, the pharmaceutical composition comprises a AS TC-E 5002 at a concentration of about 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, or 10 mM.

In some embodiments, the pharmaceutical composition comprises a KIDM inhibitor is TC-E 5002 at a unit dose of about 50 mg to 5,000 mg, about 50 mg to 4000 mg, about 50 mg to 3000 mg, about 50 mg to 2000 mg, about 50 mg to 1000 mg, about 50 mg to 500 mg, about 100 mg to 2500 mg, about 100 mg to 2000 mg, about 100 mg to 1500 mg, about 100 mg to 1000 mg, about 100 mg to 500 mg, about 200 mg to 2500 mg, about 200 mg to 2000 mg, about 200 mg to 1600 mg, about 200 mg to 1000 mg, about 100 mg, about 200 mg, about 300 mg, about 400 mg, about 500 mg, about 600 mg, about 700 mg, about 800 mg, about 900 mg, about 1000 mg, about 1200 mg, about 1400 mg, about 1600 mg, about 1800 mg, or about 2000 mg

In some embodiments, the pharmaceutical composition comprises a KDM inhibitor that is EPT-103182 at a concentration of 0.001 μM to 100 mM, about 0.01 μM to 10 mM, about 0.1 μM to 1 mM, about 1 μM to 100 μM, about 1 μM to 10 μM, 10 μM to 100 μM, or about 100 μM to 1 mM.

In some embodiments, the pharmaceutical composition comprises EPT-103182 at a concentration of about 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, or about 1 mM.

In some embodiments, the pharmaceutical composition comprises a KDM inhibitor is EPT-103182 at a unit dose of about 50 mg to 5,000 mg, about 50 mg to 4000 mg, about 50 mg to 3000 mg, about 50 mg to 2000 mg, about 50 mg to 1000 mg, about 50 mg to 500 mg, about 100 mg to 2500 mg, about 100 mg to 2000 mg, about 100 mg to 1500 mg, about 100 mg to 1000 mg, about 100 mg to 500 mg, about 150 mg to 2500 mg, about 150 mg to 2000 mg, about 150 mg to 1500 mg, about 150 mg to 1250 mg, about 75 mg, about 100 mg, about 150 mg, about 200 mg, about 300 mg, about 400 mg, about 500 mg, about 600 mg, about 700 mg, about 800 mg, about 900 mg, about 1000 mg, about 1200 mg, about 1400 mg, about 1600 mg, about 1800 mg, or about 2000 mg.

In some embodiments, the pharmaceutical composition comprises a LSD1 inhibitor that is GSK-2879552 and a GSK3 Inhibitor that is AZD1080. The GSK-2879552 is at a concentration of about 0.001 μM to 1,000 mM, about 0.01 μM to 100,000 μM, about 0.1 μM to 10,000 μM, about 1 μM to 1,000 μM, about 1 μM to 10 μM, about 10 μM to 100 μM, about 100 μM to 1 mM, or about 1 mM to 10 mM. and the AZ1090 is at a concentration of about 0.001 mM to 10,000 mM, about 0.01 mM to 1,000 mM, about 0.1 mM to 100 mM, about 0.001 mM to 0.01 mM, about 0.01 mM to 0.1 mM, about 0.1 mM to 1 mM, about 1 mM to 10 mM, about 10 mM to 100 mM, about 100 mM to 1,000 mM, or about 1,000 mM to 10,000 mM.

In some embodiments, the GSK-2879552 at a concentration of about 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, 10 mM, 12 mM, 14 mM, 16 mM, 18 mM, 20 mM, 25 mM, or about 30 mM and the AZ1090 is at a a concentration of about 1 mM, 2 mM, 3 mM, 4 mM, 5 mM, 6 mM, 7 mM, 8 mM, 9 mM, or 10 mM.

In some embodiments, the pharmaceutical composition comprises an LSD1 inhibitor that is GSK-2879552 and a GSK3 Inhibitor that is LY2090314. The GSK-2879552 is at a concentration of about 0.001 μM to 1,000 mM, about 001 μM to 100,000 μM, about 0.1 μM to 10,000 μM, about 1 μM to 1,000 μM, about 1 μM to 10 μM, about 10 μM to 100 μM, about 100 μM to 1 mM, or about 1 mM to 10 mM. and the LY2090314 at a concentration of about 0.001 μM to 10 mM, about 0.01 μM to 1 mM, about 0.1 μM to 100 uM, about 0.001 μM to 0.01 μM, about 0.01 μM to 0.1 μM, about 0.1 μM to 1 μM, about 1 μM to 10 μM, about 10 μM to 100 μM, about 100 μM to 1 mM, or about 1 mM to 10 mM.

In some embodiments, the GSK-2879552 at a concentration of about 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, 10 mM, 12 mM, 14 mM, 16 mM, 18 mM, 20 mM, 25 mM, or about 30 mM and the LY2090314 the is at a concentration of about 1 μM, 5 μM, 10 μM, 15 μM, 20 μM, or 40 μM.

In some embodiments, the pharmaceutical composition comprises an LSD1 inhibitor that is GSK-2879552 and a GSK3 Inhibitor that is that is a substituted 3-Imidazo[1,2-a]pyridin-3-yl-4-(1,2,3,4-tetrahydro-[1,4]diazepino-[6,7,1-hi]indol-7-yl)pyrrole-2,5-dione. The GSK-2879552 is at a concentration of about 0.001 μM to 1,000 mM, about 0.01 μM to 100,000 μM, about 0.1 μM to 10,000 μM, about 1 μM to 1,000 μM, about 1 μM to 10 μM, about 10 μM to 100 μM, about 100 μM to 1 mM, or about 1 mM to 10 mM. and the substituted 3-Imidazo[1,2-a]pyridin-3-yl-4-(1,2,3,4-tetrahydro-[1,4]diazepino-[6,7,1-hi]indol-7-yl)pyrrole-2,5-dione at a concentration of about 0.001 μM to 10 mM, about 0.01 μM to 1 mM, about 0.1 μM to 100 uM, about 0.001 μM to 0.01 μM, about 0.01 μM to 0.1 μM, about 0.1 μM to 1 μM, about 1 μM to 10 μM, about 10 μM to 100 μM, about 100 μM to 1 mM, or about 1 mM to 10 mM.

In some embodiments, the GSK-2879552 at a concentration of about 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM or about 10 mM and the substituted 3-Imidazo[1,2-a]pyridin-3-yl-4-(1,2,3,4-tetrahydro-[1,4]diazepino-[6,7,1-hi]indol-7-yl)pyrrole-2,5-dione, is at a concentration of about 1 μM, 5 μM, 10 μM, 15 μM, 20 μM, 50 μM, 100 μM, 250 μM or 500 μM.

In some embodiments, the pharmaceutical composition comprises a LSD1 inhibitor that is GSK-2879552 and a GSK3 Inhibitor that is GSK3-inhibitor XXII. The GSK-2879552 is at a concentration of about 0.001 μM to 1,000 mM, about 0.01 μM to 100,000 μM, about 0.1 μM to 10,000 μM, about 1 μM to 1,000 μM, about 1 μM to 10 μM, about 10 μM to 100 μM, about 100 μM to 1 mM, or about 1 mM to 10 mM. and the GSK3-inhibitor XXII, at a concentration of about of about 0.1 μM to 1,000 mM, about 1 μM to 100 mM, about 10 μM to 10 mM, about 0.1 μM to 1 μM, about 1 μM to 10 μM, about 10 μM to 100 μM, about 100 μM to 1 mM, about 1 mM to 10 mM, about 10 mM to 100 mM, or about 100 mM to 1000 mM.

In some embodiments, the GSK-2879552 at a concentration of about 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, 10 mM, 12 mM, 14 mM, 16 mM, 18 mM, 20 mM, 25 mM, or about 30 mM and the GSK3-inhibitor XXII is at a concentration of about 0.1 mM, 0.2 mM, 0.3 mM, 0.4 mM, 0.5 mM, 0.6 mM, 0.7 mM, 0.8 mM, 0.9 mM, or 1.0 mM.

In some embodiments, the pharmaceutical composition comprises an LSD1 inhibitor that is GSK-2879552 and a GSK3 Inhibitor that is CHIR99021. The GSK-2879552 is at a concentration of about 0.001 μM to 1,000 mM, about 0.01 μM to 100,000 μM, about 0.1 μM to 10,000 μM, about 1 μM to 1,000 μM, about 1 μM to 10 μM, about 10 μM to 100 μM, about 100 μM to 1 mM, or about 1 mM to 10 mM. and the CHIR99021 at a concentration of about 0.001 mM to 10,000 mM, about 0.01 mM to 1,000 mM, about 0.1 mM to 100 mM, about 0.001 mM to 0.01 mM, about 0.01 mM to 0.1 mM, about 0.1 mM to 1 mM, about 1 mM to 10 mM, about 10 mM to 100 mM, about 100 mM to 1,000 mM, or about 1,000 mM to 10,000 mM.

In some embodiments, the GSK-2879552 at a concentration of about 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, 10 mM, 12 mM, 14 mM, 16 mM, 18 mM, 20 mM, 25 mM, or about 30 mM and the CHIR99021 is at a concentration of about 1 mM, 2 mM, 3 mM, 4 mM, 5 mM, 6 mM, 7 mM, 8 mM, 9 mM, or 10 mM.

In some embodiments, the pharmaceutical composition comprises an LSD1 inhibitor that is GSK-LSD1 and a GSK3 Inhibitor that is AZD1080. The GSK-LSD1 at a concentration of about 0.001 μM to 10 mM, about 0.01 μM to 1 mM, about 0.1 μM to 100 μM, about 0.001 μM to 0.01 μM, about 0.01 μM to 0.1 μM, about 0.1 μM to 1 μM, about 1 μM to 10 μM, about 10 μM to 100 μM, or about 100 μM to 1,000 μM and the AZ1090 is at a concentration of about 0.001 mM to 10,000 mM, about 0.01 mM to 1,000 mM, about 0.1 mM to 100 mM, about 0.001 mM to 0.01 mM, about 0.01 mM to 0.1 mM, about 0.1 mM to 1 mM, about 1 mM to 10 mM, about 10 mM to 100 mM, about 100 mM to 1,000 mM, or about 1,000 mM to 10,000 mM.

In some embodiments, the is GSK-LSD1 at a concentration of about 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 1 mM, 5 mM, 10 mM, or 50 mM and the AZ1090 is at a a concentration of about 1 mM, 2 mM, 3 mM, 4 mM, 5 mM, 6 mM, 7 mM, 8 mM, 9 mM, or 10 mM.

In some embodiments, the pharmaceutical composition comprises an LSD1 inhibitor that is GSK-LSD1 and a GSK3 Inhibitor that is LY2090314. The GSK-LSD1 at a concentration of about 0.001 μM to 10 mM, about 0.01 μM to 1 mM, about 0.1 μM to 100 μM, about 0.001 μM to 0.01 μM, about 0.01 μM to 0.1 μM, about 0.1 μM to 1 μM, about 1 μM to 10 μM, about 10 μM to 100 μM, or about 100 μM to 1,000 μM and the LY2090314 at a concentration of about 0.001 μM to 10 mM, about 0.01 μM to 1 mM, about 0.1 μM to 100 uM, about 0.001 μM to 0.01 μM, about 0.01 μM to 0.1 μM, about 0.1 μM to 1 μM, about 1 μM to 10 μM, about 10 μM to 100 μM, about 100 μM to 1 mM, or about 1 mM to 10 mM.

In some embodiments, the GSK-LSD1 at a concentration of about 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 1 mM, 5 mM, 10 mM, or 50 mM and the LY2090314 the is at a concentration of about 1 μM, 5 μM, 10 μM, 15 μM, 20 μM, or 30 μM.

In some embodiments, the pharmaceutical composition comprises an LSD1 inhibitor that is GSK-LSD1 and a GSK3 Inhibitor that is that is a substituted 3-Imidazo[1,2-a]pyridin-3-yl-4-(1,2,3,4-tetrahydro-[1,4]diazepino-[6,7,1-hi]indol-7-yl)pyrrole-2,5-dione. The GSK-LSD1 at a concentration of about 0.001 μM to 10 mM, about 0.01 μM to 1 mM, about 0.1 μM to 100 μM, about 0.001 μM to 0.01 μM, about 0.01 μM to 0.1 μM, about 0.1 μM to 1 μM, about 1 μM to 10 μM, about 10 μM to 100 μM, or about 100 μM to 1,000 μM and the substituted 3-Imidazo[1,2-a]pyridin-3-yl-4-(1,2,3,4-tetrahydro-[1,4]diazepino-[6,7,1-hi]indol-7-yl)pyrrole-2,5-dione at a concentration of about 0.001 μM to 10 mM, about 0.01 μM to 1 mM, about 0.1 μM to 100 uM, about 0.001 μM to 0.01 μM, about 0.01 μM to 0.1 μM, about 0.1 μM to 1 μM, about 1 μM to 10 μM, about 10 μM to 100 μM, about 100 μM to 1 mM, or about 1 mM to 10 mM.

In some embodiments, the GSK-LSD1 at a concentration of about 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 1 mM, 5 mM, 10 mM, or 50 mM and the substituted 3-Imidazo[1,2-a]pyridin-3-yl-4-(1,2,3,4-tetrahydro-[1,4]diazepino-[6,7,1-hi]indol-7-yl)pyrrole-2,5-dione, is at a concentration of about 1 μM, 5 μM, 10 μM, 15 μM, 20 μM, 50 μM, 100 μM, 250 μM or 500 μM.

In some embodiments, the pharmaceutical composition comprises an LSD1 inhibitor that is GSK-LSD1 and a GSK3 Inhibitor that is GSK3-inhibitor XXII. The GSK-LSD1 at a concentration of about 0.001 μM to 10 mM, about 0.01 μM to 1 mM, about 0.1 μM to 100 μM, about 0.001 μM to 0.01 μM, about 0.01 μM to 0.1 μM, about 0.1 μM to 1 μM, about 1 μM to 10 μM, about 10 μM to 100 μM, or about 100 μM to 1,000 μM. and the GSK3-inhibitor XXII, at a concentration of about of about 0.1 μM to 1,000 mM, about 1 μM to 100 mM, about 10 μM to 10 mM, about 0.1 μM to 1 μM, about 1 μM to 10 μM, about 10 μM to 100 μM, about 100 μM to 1 mM, about 1 mM to 10 mM, about 10 mM to 100 mM, or about 100 mM to 1000 mM.

In some embodiments, the GSK-LSD1 at a concentration of about 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 1 mM, 5 mM, 10 mM, or 50 mM and the GSK3-inhibitor XXII is at a concentration of about 0.1 mM, 0.2 mM, 0.3 mM, 0.4 mM, 0.5 mM, 0.6 mM, 0.7 mM, 0.8 mM, 0.9 mM, or 1.0 mM.

In some embodiments, the pharmaceutical composition comprises an LSD1 inhibitor that is GSK-LSD1 and a GSK3 Inhibitor that is CHIR99021. The GSK-LSD1 at a concentration of about 0.001 μM to 10 mM, about 0.01 μM to 1 mM, about 0.1 μM to 100 μM, about 0.001 μM to 0.01 μM, about 0.01 μM to 0.1 μM, about 0.1 μM to 1 μM, about 1 μM to 10 μM, about 10 μM to 100 μM, or about 100 μM to 1,000 μM and the CHIR99021 at a concentration of about 0.001 mM to 10,000 mM, about 0.01 mM to 1,000 mM, about 0.1 mM to 100 mM, about 0.001 mM to 0.01 mM, about 0.01 mM to 0.1 mM, about 0.1 mM to 1 mM, about 1 mM to 10 mM, about 10 mM to 100 mM, about 100 mM to 1,000 mM, or about 1,000 mM to 10,000 mM.

In some embodiments, the GSK-LSD1 at a concentration of about 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 1 mM, 5 mM, 10 mM, or 50 mM and the CHIR99021 is at a concentration of about 1 mM, 2 mM, 3 mM, 4 mM, 5 mM, 6 mM, 7 mM, 8 mM, 9 mM, or 10 mM.

In some embodiments, the pharmaceutical composition comprises an LSD1 inhibitor that is Tranylcypromine and a GSK3 Inhibitor that is AZD1080. The Tranylcypromine at a concentration of about 0.001 mM to 10,000 mM, about 0.01 mM to 1,000 mM, about 0.1 mM to 100 mM, about 0.001 mM to 0.01 mM, about 0.01 mM to 0.1 mM, about 0.1 mM to 1 mM, about 1 mM to 10 mM, about 10 mM to 100 mM, about 100 mM to 1,000 mM, or about 1,000 mM to 10,000 mM. and the AZ1090 is at a concentration of about 0.001 mM to 10,000 mM, about 0.01 mM to 1,000 mM, about 0.1 mM to 100 mM, about 0.001 mM to 0.01 mM, about 0.01 mM to 0.1 mM about 0.1 mM to 1 mM, about 1 mM to 10 mM, about 10 mM to 100 mM, about 100 mM to 1,000 mM, or about 1,000 mM to 10,000 mM.

In some embodiments, Tranylcypromine at a concentration of about 0.1 mM, 0.2 mM, 0.3 mM, 0.4 mM, 0.5 mM, 0.6 mM, 0.7 mM, 0.8 mM, 0.9 mM, 1 mM, 2 mM, 3 mM, 4 mM, 5 mM, 6 mM, 7 mM, 8 mM, 9 mM, 10 mM, 12 mM, 14 mM, 16 mM, 18 mM, or 20 mM and the AZ1090 is at a a concentration of about 1 mM, 2 mM, 3 mM, 4 mM, 5 mM, 6 mM, 7 mM, 8 mM, 9 mM, or 10 mM.

In some embodiments, the pharmaceutical composition comprises an LSD1 inhibitor that is Tranylcypromine and a GSK3 Inhibitor that is LY2090314. The Tranylcypromine at a concentration of about 0.001 mM to 10,000 mM, about 0.01 mM to 1,000 mM, about 0.1 mM to 100 mM, about 0.001 mM to 0.01 mM, about 0.01 mM to 0.1 mM, about 0.1 mM to 1 mM, about 1 mM to 10 mM, about 10 mM to 100 mM, about 100 mM to 1,000 mM, or about 1,000 mM to 10,000 mM and the LY2090314 at a concentration of about 0.001 μM to 10 mM, about 0.01 μM to 1 mM, about 0.1 μM to 100 uM, about 0.001 μM to 001 μM, about 0.01 μM to 0.1 μM, about 0.1 μM to 1 μM, about 1 μM to 10 μM, about 10 μM to 100 μM, about 100 μM to 1 mM, or about 1 mM to 10 mM.

In some embodiments, Tranylcypromine at a concentration of about 0.1 mM, 0.2 mM, 0.3 mM, 0.4 mM, 0.5 mM, 0.6 mM, 0.7 mM, 0.8 mM, 0.9 mM, 1 mM, 2 mM, 3 mM, 4 mM, 5 mM, 6 mM, 7 mM, 8 mM, 9 mM, 10 mM, 12 mM, 14 mM, 16 mM, 18 mM, or 20 mM and the LY2090314 the is at a concentration of about 1 μM, 5 μM, 10 μM, 15 μM, 20 μM, or 40 μM.

In some embodiments, the pharmaceutical composition comprises an LSD1 inhibitor that is Tranylcypromine and a GSK3 Inhibitor that is that is a substituted 3-Imidazo[1,2-a]pyridin-3-yl-4-(1,2,3,4-tetrahydro-[1,4]diazepino-[6,7,1-hi]indol-7-yl)pyrrole-2,5-dione. The Tranylcypromine at a concentration of about 0.001 mM to 10,000 mM, about 0.01 mM to 1,000 mM, about 0.1 mM to 100 mM, about 0.001 mM to 0.01 mM, about 0.01 mM to 0.1 mM, about 0.1 mM to 1 mM, about 1 mM to 10 mM, about 10 mM, to 100 mM, about 100 mM to 1,000 mM, or about 1,000 mM to 10,000 mM, and the substituted 3-Imidazo[1,2-a]pyridin-3-yl-4-(1,2,3,4-tetrahydro-[1,4]diazepino-[6,7,1-hi]indol-7-yl)pyrrole-2,5-dione at a concentration of about 0.001 μM to 10 mM, about 0.01 μM to 1 mM, about 0.1 μM to 100 uM, about 0.001 μM to 0.01 μM, about 0.01 μM to 0.1 μM, about 0.1 μM to 1 μM, about 1 μM to 10 μM, about 10 μM to 100 μM, about 100 μM to 1 mM, or about 1 mM to 10 mM.

In some embodiments, Tranylcypromine at a concentration of about 0.1 mM, 0.2 mM, 0.3 mM, 0.4 mM, 0.5 mM, 0.6 mM, 0.7 mM, 0.8 mM, 0.9 mM, 1 mM, 2 mM, 3 mM, 4 mM, 5 mM, 6 mM, 7 mM, 8 mM, 9 mM, 10 mM, 12 mM, 14 mM, 16 mM, 18 mM, or 20 mM and the substituted 3-Imidazo[1,2-a]pyridin-3-yl-4-(1,2,3,4-tetrahydro-[1,4]diazepino-[6,7,1-hi]indol-7-yl)pyrrole-2,5-dione, is at a concentration of about 1 μM, 5 μM, 10 μM, 15 μM, 20 μM, 50 μM, 100 μM, 250 μM or 500 μM.

In some embodiments, the pharmaceutical composition comprises an LSD1 inhibitor that is Tranylcypromine and a GSK3 Inhibitor that is GSK3-inhibitor XXII. Tranylcypromine at a concentration of about 0.001 mM to 10,000 mM, about 0.01 mM to 1,000 mM, about 0.1 mM to 100 mM, about 0.001 mM to 0.01 mM, about 0.01 mM to 0.1 mM, about 0.1 mM to 1 mM, about 1 mM to 10 mM, about 10 mM to 100 mM, about 100 mM to 1,000 mM, or about 1,000 mM to 10,000 mM and the GSK3-inhibitor XXII, at a concentration of about of about 0.1 μM to 1,000 mM, about 1 μM to 100 mM, about 10 μM to 10 mM, about 0.1 μM to 1 μM, about 1 μM to 10 μM, about 10 μM to 100 μM, about 100 μM to 1 mM, about 1 mM to 10 mM, about 10 mM to 100 mM, or about 100 mM to 1000 mM.

In some embodiments, Tranylcypromine at a concentration of about 0.1 mM, 0.2 mM, 0.3 mM, 0.4 mM, 0.5 mM, 0.6 mM, 0.7 mM, 0.8 mM, 0.9 mM, 1 mM, 2 mM, 3 mM, 4 mM, 5 mM, 6 mM, 7 mM, 8 mM, 9 mM, 10 mM, 12 mM, 14 mM, 16 mM, 18 mM, or 20 mM and the GSK3-inhibitor XXII is at a concentration of about 0.1 mM, 0.2 mM, 0.3 mM, 0.4 mM, 0.5 mM, 0.6 mM, 0.7 mM, 0.8 mM, 0.9 mM, or 1.0 mM.

In some embodiments, the pharmaceutical composition comprises an LSD1 inhibitor that is Tranylcypromine and a GSK3 Inhibitor that is CHIR99021. The Tranylcypromine at a concentration of about 0.001 mM to 10,000 mM, about 0.01 mM to 1,000 mM, about 0.1 mM to 100 mM, about 0.001 mM to 0.01 mM, about 0.01 mM to 0.1 mM, about 0.1 mM to 1 mM, about 1 mM to 10 mM, about 10 mM to 100 mM, about 100 mM to 1,000 mM, or about 1,000 mM to 10,000 mM. and the CHIR99021 at a concentration of about 0.001 mM to 10,000 mM, about 0.01 mM to 1,000 mM, about 0.1 mM to 100 mM, about 0.001 mM to 0.01 mM, about 0.01 mM to 0.1 mM, about 0.1 mM to 1 mM, about 1 mM to 10 mM, about 10 mM to 100 mM, about 100 mM to 1,000 mM, or about 1,000 mM to 10,000 mM.

Tranylcypromine at a concentration of about 0.1 mM, 0.2 mM, 0.3 mM, 0.4 mM, 0.5 mM, 0.6 mM, 0.7 mM, 0.8 mM, 0.9 mM, 1 mM, 2 mM, 3 mM, 4 mM, 5 mM, 6 mM, 7 mM, 8 mM, 9 mM, 10 mM, 12 mM, 14 mM, 16 mM, 18 mM, or 20 mM and the CHIR99021 is at a concentration of about 1 mM, 2 mM, 3 mM, 4 mM, 5 mM, 6 mM, 7 mM, 8 mM, 9 mM, or 10 mM.

In some embodiments, the pharmaceutical composition comprises an LSD1 inhibitor that is Phenelzine sulfate and a GSK3 Inhibitor that is AZD1080. The Phenelzine sulfate at a concentration of about 0.001 mM to 100,000 mM, 0.01 mM to 10,000 mM, about 0.1 mM to 1,000 mM, about 0.1 mM to 100 mM, about 0.001 mM to 0.01 mM, about 0.01 mM to 0.1 mM, about 0.1 mM to 1 mM, about 1 mM to 10 mM, about 10 mM to 100 mM, about 100 mM to 1,000 mM, or about 1,000 mM to 10,000 mM, and the AZ1090 is at a concentration of about 0.001 mM to 10,000 mM, about 0.01 mM to 1,000 mM, about 0.1 mM to 100 mM, about 0.001 mM to 0.01 mM, about 0.01 mM to 0.1 mM, about 0.1 mM to 1 mM, about 1 mM to 10 mM, about 10 mM to 100 mM, about 100 mM to 1,000 mM, or about 1,000 mM to 10,000 mM.

In some embodiments, at a concentration of about 0.1 mM, 0.2 mM, 0.3 mM, 0.4 mM, 0.5 mM, 0.6 mM, 0.7 mM, 0.8 mM, 0.9 mM, 1 mM, 2 mM, 3 mM, 4 mM, 5 mM, 6 mM, 7 mM, 8 mM, 9 mM, or 10 mM. and the AZ1090 is at a a concentration of about 1 mM, 2 mM, 3 mM, 4 mM, 5 mM, 6 mM, 7 mM, 8 mM, 9 mM, or 10 mM.

In some embodiments, the pharmaceutical composition comprises a LSD1 inhibitor that is Phenelzine sulfate and a GSK3 Inhibitor that is LY2090314. The Phenelzine sulfate at a concentration of about 0.001 mM to 100,000 mM, 0.01 mM to 10,000 mM, about 0.1 mM to 1,000 mM, about 0.1 mM to 100 mM, about 0.001 mM to 0.01 mM, about 0.01 mM to 0.1 mM, about 0.1 mM to 1 mM, about 1 mM to 10 mM, about 10 mM to 100 mM, about 100 mM to 1,000 mM, or about 1,000 mM to 10,000 mM. and the LY2090314 at a concentration of about 0.001 μM to 10 mM, about 0.01 μM to 1 mM, about 0.1 μM to 100 uM, about 0.001 μM to 0.01 μM, about 0.01 μM to 0.1 μM, about 0.1 μM to 1 μM, about 1 μM to 10 μM, about 10 μM to 100 μM, about 100 μM to 1 mM, or about 1 mM to 10 mM at a concentration of about 0.1 mM, 0.2 mM, 0.3 mM, 0.4 mM, 0.5 mM, 0.6 mM, 0.7 mM, 0.8 mM, 0.9 mM, 1 mM, 2 mM, 3 mM, 4 mM, 5 mM, 6 mM, 7 mM, 8 mM, 9 mM, or 10 mM and the LY2090314 the is at a concentration of about 1 μM, 5 μM, 10 μM, 15 μM, 20 μM, or 40 μM.

In some embodiments, the pharmaceutical composition comprises an LSD1 inhibitor that is Phenelzine sulfate and a GSK3 Inhibitor that is that is a substituted 3-imidazo[1,2-a]pyridin-3-yl-4-(1,2,3,4-tetrahydro-[1,4]diazepino-[6,7,1-hi]indol-7-yl)pyrrole-2,5-dione. The Phenelzine sulfate at a concentration of about 0.001 mM to 100,000 mM, 0.01 mM to 10,000 mM, about 0.1 mM to 1,000 mM, about 0.1 mM to 100 mM, about 0.001 mM to 0.01 mM, about 0.01 mM to 0.1 mM, about 0.1 mM to 1 mM, about 1 mM to 10 mM, about 10 mM to 100 mM, about 100 mM to 1,000 mM, or about 1,000 mM to 10,000 mM. and the substituted 3-Imidazo[1,2-a]pyridin-3-yl-4-(1,2,3,4-tetrahydro-[1,4]diazepino-[6,7,1-hi]indol-7-yl)pyrrole-2,5-dione at a concentration of about 0.001 μM to 10 mM, about 0.01 μM to 1 mM, about 0.1 μM to 100 uM, about 0.001 μM to 0.01 μM, about 0.01 μM to 0.1 μM, about 0.1 μM to 1 μM, about 1 μM to 10 μM, about 10 μM to 100 μM, about 100 μM to 1 mM, or about 1 mM to 10 mM.

In some embodiments, at a concentration of about 0.1 mM, 0.2 mM, 0.3 mM, 0.4 mM, 0.5 mM, 0.6 mM, 0.7 mM, 0.8 mM, 0.9 mM, 1 mM, 2 mM, 3 mM, 4 mM, 5 mM, 6 mM, 7 mM, 8 mM, 9 mM, or 10 mM and the substituted 3-Imidazo[1,2-a]pyridin-3-yl-4-(1,2,3,4-tetrahydro-[1,4]diazepino-[6,7,1-hi]indol-7-yl)pyrrole-2,5-dione, is at a concentration of about 1 μM, 5 μM, 10 μM, 15 μM, 20 μM, 50 μM, 100 μM, 250 μM or 500 μM.

In some embodiments, the pharmaceutical composition comprises an LSD1 inhibitor that is Phenelzine sulfate and a GSK3 Inhibitor that is GSK3-inhibitor XXII. The Phenelzine sulfate at a concentration of about 0.001 mM to 100,000 mM, 0.01 mM to 10,000 mM, about 0.1 mM to 1,000 mM, about 0.1 mM to 100 mM, about 0.001 mM to 0.01 mM, about 0.01 mM to 0.1 mM, about 0.1 mM to 1 mM, about 1 mM to 10 mM, about 10 mM to 100 mM, about 100 mM to 1,000 mM, or about 1,000 mM to 10,000 mM. and the GSK3-inhibitor XXII, at a concentration of about of about 0.1 μM to 1,000 mM, about 1 μM to 100 mM, about 10 μM to 10 mM, about 0.1 μM to 1 μM, about 1 μM to 10 μM, about 10 μM to 100 μM, about 100 μM to 1 mM, about 1 mM to 10 mM, about 10 mM to 100 mM, or about 100 mM to 1000 mM.

In some embodiments, at a concentration of about 0.1 mM, 0.2 mM, 0.3 mM, 0.4 mM, 0.5 mM, 0.6 mM, 0.7 mM, 0.8 mM, 0.9 mM, 1 mM, 2 mM, 3 mM, 4 mM, 5 mM, 6 mM, 7 mM, 8 mM, 9 mM, or 10 mM. and the GSK3-inhibitor XXII is at a concentration of about 0.1 mM, 0.2 mM, 0.3 mM, 0.4 mM, 0.5 mM, 0.6 mM, 0.7 mM, 0.8 mM, 0.9 mM, or 1.0 mM.

In some embodiments, the pharmaceutical composition comprises an LSD1 inhibitor that is Phenelzine sulfate and a GSK3 Inhibitor that is CHIR99021. The Phenelzine sulfate at a concentration of about 0.001 mM to 100,000 mM, 0.01 mM to 10,000 mM, about 0.1 mM to 1,000 mM, about 0.1 mM to 100 mM, about 0.001 mM to 0.01 mM, about 0.01 mM to 0.1 mM, about 0.1 mM to 1 mM, about 1 mM to 10 mM, about 10 mM to 100 mM, about 100 mM to 1,000 mM, or about 1,000 mM to 10,000 mM. and the CHIR99021 at a concentration of about 0.001 mM to 10,000 mM, about 0.01 mM to 1,000 mM, about 0.1 mM to 100 mM, about 0.001 mM to 0.01 mM, about 0.01 mM to 0.1 mM, about 0.1 mM to 1 mM, about 1 mM to 10 mM, about 10 mM to 100 mM, about 100 mM to 1,000 mM, or about 1,000 mM to 10.000 mM.

In some embodiments, at a concentration of about 0.1 mM, 0.2 mM, 0.3 mM, 0.4 mM, 0.5 mM, 0.6 mM, 0.7 mM, 0.8 mM, 0.9 mM, 1 mM, 2 mM, 3 mM, 4 mM, 5 mM, 6 mM, 7 mM, 8 mM, 9 mM, or 10 mM and the CHIR99021 is at a concentration of about 1 mM, 2 mM, 3 mM, 4 mM, 5 mM, 6 mM, 7 mM, 8 mM, 9 mM, or 10 mM.

In some embodiments, the pharmaceutical composition comprises an LSD1 inhibitor that is ORY-1001 and a GSK3 Inhibitor that is AZD1080. The ORY-1001 at a concentration of about 0.001 mM to 100,000 mM, 0.01 mM to 10,000 mM, about 0.1 mM to 1,000 mM, about 0.1 mM to 100 mM, about 0.001 mM to 0.01 mM, about 0.01 mM to 0.1 mM, about 0.1 mM to 1 mM, about 1 mM to 10 mM, about 10 mM to 100 mM, about 100 mM to 1,000 mM, or about 1,000 mM to 10,000 mM. and the AZ1090 is at a concentration of about 0.001 mM to 10.000 mM, about 0.01 mM to 1,000 mM, about 0.1 mM to 100 mM, about 0.001 mM to 0.01 mM, about 0.01 mM to 0.1 mM, about 0.1 mM to 1 mM, about 1 mM to 10 mM, about 10 mM to 100 mM, about 100 mM to 1,000 mM, or about 1,000 mM to 10,000 mM.

In some embodiments, at a concentration of about 0.1 mM, 0.2 mM, 0.3 mM, 0.4 mM, 0.5 mM, 0.6 mM, 0.7 mM, 0.8 mM, 0.9 mM, 1 mM, 2 mM, 3 mM, 4 mM, 5 mM, 6 mM, 7 mM, 8 mM, 9 mM, or 10 mM. and the AZ1090 is at a a concentration of about 1 mM, 2 mM, 3 mM, 4 mM, 5 mM, 6 mM, 7 mM, 8 mM, 9 mM, or 10 mM.

In some embodiments, the pharmaceutical composition comprises a LSD1 inhibitor that is ORY-1001 and a GSK3 Inhibitor that is LY2090314. The ORY-1001 at a concentration of about 0.001 mM to 100,000 mM, 0.01 mM to 10,000 mM, about 0.1 mM to 1,000 mM, about 0.1 mM to 100 mM, about 0.001 mM to 0.01 mM, about 0.01 mM to 0.1 mM, about 0.1 mM to 1 mM, about 1 mM to 10 mM, about 10 mM to 100 mM, about 100 mM to 1,000 mM, or about 1,000 mM to 10,000 mM. and the LY2090314 at a concentration of about 0.001 μM to 10 mM, about 0.01 μM to 1 mM, about 0.1 μM to 100 uM, about 0.001 μM to 0.01 μM, about 0.01 μM to 0.1 μM, about 0.1 μM to 1 μM, about 1 μM to 10 μM, about 10 μM to 100 μM, about 100 μM to 1 mM, or about 1 mM to 10 mM at a concentration of about 0.1 mM, 0.2 mM, 0.3 mM, 0.4 mM, 0.5 mM, 0.6 mM, 0.7 mM, 0.8 mM, 0.9 mM, 1 mM, 2 mM, 3 mM, 4 mM, 5 mM, 6 mM, 7 mM, 8 mM, 9 mM, or 10 mM and the LY2090314 the is at a concentration of about 1 μM, 5 μM, 10 μM, 15 μM, 20 μM, or 40 μM.

In some embodiments, the pharmaceutical composition comprises an LSD1 inhibitor that is ORY-1001 and a GSK3 Inhibitor that is that is a substituted 3-Imidazo[1,2-a]pyridin-3-yl-4-(1,2,3,4-tetrahydro-[1,4]diazepino-[6,7,1-hi]indol-7-yl)pyrrole-2,5-dione. The ORY-1001 at a concentration of about 0.001 mM to 100,000 mM, 0.01 mM to 10,000 mM, about 0.1 mM to 1,000 mM, about 0.1 mM to 100 mM, about 0.001 mM to 0.01 mM, about 0.01 mM to 0.1 mM, about 0.1 mM to 1 mM, about 1 mM to 10 mM, about 10 mM to 100 mM, about 100 mM to 1,000 mM, or about 1,000 mM to 10,000 mM. and the substituted 3-Imidazo[1,2-a]pyridin-3-yl-4-(1,2,3,4-tetrahydro-[1,4]diazepino-[6,7,1-hi]indol-7-yl)pyrrole-2,5-dione at a concentration of about 0.001 μM to 10 mM, about 0.01 μM to 1 mM, about 0.1 μM to 100 uM, about 0.001 μM to 0.01 μM, about 0.01 μM to 0.1 μM, about 0.1 μM to 1 μM, about 1 μM to 10 μM, about 10 μM to 100 μM, about 100 μM to 1 mM, or about 1 mM to 10 mM.

In some embodiments, at a concentration of about 0.1 mM, 0.2 mM, 0.3 mM, 0.4 mM, 0.5 mM, 0.6 mM, 0.7 mM, 0.8 mM, 0.9 mM, 1 mM, 2 mM, 3 mM, 4 mM, 5 mM, 6 mM, 7 mM, 8 mM, 9 mM, or 10 mM and the substituted 3-Imidazo[1,2-a]pyridin-3-yl-4-(1,2,3,4-tetrahydro-[1,4]diazepino-[6,7,1-hi]indol-7-yl)pyrrole-2,5-dione, is at a concentration of about 1 μM, 5 μM, 10 μM, 15 μM, 20 μM, 50 μM, 100 μM, 250 μM or 500 μM.

In some embodiments, the pharmaceutical composition comprises an LSD1 inhibitor that is ORY-1001 and a GSK3 Inhibitor that is GSK3-inhibitor XXII. The ORY-1001 at a concentration of about 0.1 mM to 100,000 mM, 0.01 mM to 10,000 mM, about 0.1 mM to 1,000 mM, about 0.001 mM to 100 mM, about 0.001 mM to 0.01 mM, about 0.01 mM to 0.1 mM, about 0.1 mM to 1 mM, about 1 mM to 10 mM, about 10 mM to 100 mM, about 100 mM to 1,000 mM, or about 1,000 mM to 10,000 mM. and the GSK3-inhibitor XXII, at a concentration of about of about 0.1 μM to 1,000 mM, about 1 μM to 100 mM, about 10 μM to 10 mM, about 0.1 μM to 1 μM, about 1 μM to 10 μM, about 10 μM to 100 μM, about 100 μM to 1 mM, about 1 mM to 10 mM, about 10 mM to 100 mM, or about 100 mM to 1000 mM.

In some embodiments, at a concentration of about 0.1 mM, 0.2 mM, 0.3 mM, 0.4 mM, 0.5 mM, 0.6 mM, 0.7 mM, 0.8 mM, 0.9 mM, 1 mM, 2 mM, 3 mM, 4 mM, 5 mM, 6 mM, 7 mM, 8 mM, 9 mM, or 10 mM. and the GSK3-inhibitor XXII is at a concentration of about 0.1 mM, 0.2 mM, 0.3 mM, 0.4 mM, 0.5 mM, 0.6 mM, 0.7 mM, 0.8 mM, 0.9 mM, or 1.0 mM.

In some embodiments, the pharmaceutical composition comprises an LSD1 inhibitor that is ORY-1001 and a GSK3 Inhibitor that is CHIR99021. The ORY-1001 at a concentration of about 0.001 mM to 100,000 mM, 0.01 mM to 10,000 mM, about 0.1 mM to 1,000 mM, about 0.1 mM to 100 mM, about 0.001 mM to 0.01 mM, about 0.01 mM to 0.1 mM, about 0.1 mM to 1 mM, about 1 mM to 10 mM, about 10 mM to 100 mM, about 100 mM to 1,000 mM, or about 1,000 mM to 10,000 mM, and the CHIR99021 at a concentration of about 0.001 mM to 10,000 mM, about 0.01 mM to 1,000 mM, about 0.1 mM to 100 mM, about 0.001 mM to 0.01 mM, about 0.01 mM to 0.1 mM, about 0.1 mM to 1 mM, about 1 mM to 10 mM, about 10 mM to 100 mM, about 100 mM to 1,000 mM, or about 1,000 mM to 10,000 mM.

In some embodiments, at a concentration of about 0.1 mM, 0.2 mM, 0.3 mM, 0.4 mM, 0.5 mM, 0.6 mM, 0.7 mM, 0.8 mM, 0.9 mM, 1 mM, 2 mM, 3 mM, 4 mM, 5 mM, 6 mM, 7 mM, 8 mM, 9 mM, or 10 mM and the CHIR99021 is at a concentration of about 1 mM, 2 mM, 3 mM, 4 mM, 5 mM, 6 mM, 7 mM, 8 mM, 9 mM, or 10 mM.

In some embodiments, the pharmaceutical composition comprises an LSD1 inhibitor that is GSK-2879552 and a GSK3 Inhibitor that is AZD1080 and a HDAC inhibitor that is VPA. The GSK-2879552 is at a concentration of about 0.001 μM to 1,000 mM, about 0.01 μM to 100,000 μM, about 0.1 μM to 10,000 μM, about 1 μM to 1,000 μM, about 1 μM to 10 μM, about 10 μM to 100 μM, about 100 μM to 1 mM, or about 1 mM to 10 mM. and the AZ1090 is at a concentration of about 0.001 mM to 10,000 mM, about 0.01 mM to 1,000 mM, about 0.1 mM to 100 mM, about 0.001 mM to 0.01 mM, about 0.01 mM to 0.1 mM, about 0.1 mM to 1 mM, about 1 mM to 10 mM, about 10 mM to 100 mM, about 100 mM to 1,000 mM, or about 1,000 mM to 10,000 mM and the VPA at a concentration about 100 mM to 4,000 mM.

In some embodiments, the GSK-2879552 at a concentration of about 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, 10 mM, 12 mM, 14 mM, 16 mM, 18 mM, 20 mM, 25 mM, or about 30 mM and the AZ1090 is at a a concentration of about 1 mM, 2 mM, 3 mM, 4 mM, 5 mM, 6 mM, 7 mM, 8 mM, 9 mM, or 10 mM and the VPA at a concentration about 100 mM to 4,000 mM.

In some embodiments, the pharmaceutical composition comprises an LSD1 inhibitor that is RN-1 and a GSK3 Inhibitor that is AZD1080. The RN-1 at a concentration of about 0.1 mM to 100,000 mM, 0.01 mM to 10,000 mM, about 0.1 mM to 1,000 mM, about 0.1 mM to 100 mM, about 0.001 mM to 0.01 mM, about 0.01 mM to 0.1 mM, about 0.1 mM to 1 mM, about 1 mM to 10 mM, about 10 mM to 100 mM, about 100 mM to 1,000 mM, or about 1,000 mM to 10,000 mM. and the AZ1090 is at a concentration of about 0.001 mM to 10,000 mM, about 0.01 mM to 1,000 mM, about 0.1 mM to 100 mM, about 0.001 mM to 0.01 mM, about 0.01 mM to 0.1 mM, about 0.1 mM to 1 mM, about 1 mM to 10 mM, about 10 mM to 100 mM, about 100 mM to 1,000 mM, or about 1,000 mM to 10,000 mM.

In some embodiments, at a concentration of about 0.1 mM, 0.2 mM, 0.3 mM, 0.4 mM, 0.5 mM, 0.6 mM, 0.7 mM, 0.8 mM, 0.9 mM, 1 mM, 2 mM, 3 mM, 4 mM, 5 mM, 6 mM, 7 mM, 8 mM, 9 mM, or 10 mM. and the AZ1090 is at a a concentration of about 1 mM, 2 mM, 3 mM, 4 mM, 5 mM, 6 mM, 7 mM, 8 mM, 9 mM, or 10 mM.

In some embodiments, the pharmaceutical composition comprises a LSD1 inhibitor that is KN-1 and a GSK3 Inhibitor that is LY2090314. The RN-1 at a concentration of about 0.001 mM to 100,000 mM, 0.01 mM to 10,000 mM, about 0.1 mM to 1,000 mM, about 0.1 mM to 100 mM, about 0.001 mM to 0.01 mM, about 0.01 mM to 0.1 mM, about 0.1 mM to 1 mM, about 1 mM to 10 mM, about 10 mM to 100 mM, about 100 mM to 1,000 mM, or about 1,000 mM to 10,000 mM. and the LY2090314 at a concentration of about 0.001 μM to 10 mM, about 0.01 μM to 1 mM, about 0.1 μM to 100 uM, about 0.001 μM to 0.01 μM, about 0.01 μM to 0.1 μM, about 0.1 μM to 1 μM, about 1 μM to 10 μM, about 10 μM to 100 μM, about 100 μM to 1 mM, or about 1 mM to 10 mM at a concentration of about 0.1 mM, 0.2 mM, 0.3 mM, 0.4 mM, 0.5 mM, 0.6 mM, 0.7 mM, 0.8 mM, 0.9 mM, 1 mM, 2 mM, 3 mM, 4 mM, 5 mM, 6 mM, 7 mM, 8 mM, 9 mM, or 10 mM and the LY2090314 the is at a concentration of about 1 μM, 5 μM, 10 μM, 15 μM, 20 μM, or 40 μM.

In some embodiments, the pharmaceutical composition comprises an LSD1 inhibitor that is RN-1 and a GSK3 Inhibitor that is that is a substituted 3-Imidazo[1,2-a]pyridin-3-yl-4-(1,2,3,4-tetrahydro-[1,4]diazepino-[6,7,1-hi]indol-7-yl)pyrrole-2,5-dione. The RN-1 at a concentration of about 0.001 mM to 100,000 mM, 0.01 mM to 10,000 mM, about 0.1 mM to 1,000 mM, about 0.1 mM to 100 mM, about 0.001 mM to 0.01 mM, about 0.01 mM to 0.1 mM, about 0.1 mM to 1 mM, about 1 mM to 10 mM, about 10 mM to 100 mM, about 100 mM to 1,000 mM, or about 1,000 mM to 10,000 mM. and the substituted 3-Imidazo[1,2-a]pyridin-3-yl-4-(1,2,3,4-tetrahydro-[1,4]diazepino-[6,7,1-hi]indol-7-yl)pyrrole-2,5-dione at a concentration of about 0.001 μM to 10 mM, about 0.01 μM to 1 mM, about 0.1 μM to 100 uM, about 0.001 μM to 0.01 μM, about 0.01 μM to 0.1 μM, about 0.1 μM to 1 μM, about 1 μM to 10 μM, about 10 μM to 100 μM, about 100 μM to 1 mM, or about 1 mM to 10 mM.

In some embodiments, at a concentration of about 0.1 mM, 0.2 mM, 0.3 mM, 0.4 mM, 0.5 mM, 0.6 mM, 0.7 mM, 0.8 mM, 0.9 mM, 1 mM, 2 mM, 3 mM, 4 mM, 5 mM, 6 mM, 7 mM, 8 mM, 9 mM, or 10 mM and the substituted 3-Imidazo[1,2-a]pyridin-3-yl-4-(1,2,3,4-tetrahydro-[1,4]diazepino-[6,7,1-hi]indol-7-yl)pyrrole-2,5-dione, is at a concentration of about 1 μM, 5 μM, 10 μM, 15 μM, 20 μM, 50 μM, 100 μM, 250 μM or 500 μM.

In some embodiments, the pharmaceutical composition comprises an LSD1 inhibitor that is RN-1 and a GSK3 Inhibitor that is GSK3-inhibitor XXII. The RN-1 at a concentration of about 0.001 mM to 100,000 mM, 0.01 mM to 10,000 mM, about 0.1 mM to 1,000 mM, about 0.1 mM to 100 mM, about 0.001 mM to 0.01 mM, about 0.01 mM to 0.1 mM, about 0.1 mM to 1 mM, about 1 mM to 10 mM, about 10 mM to 100 mM, about 100 mM to 1,000 mM, or about 1,000 mM to 10,000 mM. and the GSK3-inhibitor XXII, at a concentration of about of about 0.1 μM to 1,000 mM, about 1 μM to 100 mM, about 10 μM to 10 mM, about 0.1 μM to 1 μM, about 1 μM to 10 μM, about 10 μM to 100 μM, about 100 μM to 1 mM, about 1 mM to 10 mM, about 10 mM to 100 mM, or about 100 mM to 1000 mM.

In some embodiments, at a concentration of about 0.1 mM, 0.2 mM, 0.3 mM, 0.4 mM, 0.5 mM, 0.6 mM, 0.7 mM, 0.8 mM, 0.9 mM, 1 mM, 2 mM, 3 mM, 4 mM, 5 mM, 6 mM, 7 mM, 8 mM, 9 mM, or 10 mM. and the GSK3-inhibitor XXII is at a concentration of about 0.1 mM, 0.2 mM, 0.3 mM, 0.4 mM, 0.5 mM, 0.6 mM, 0.7 mM, 0.8 mM, 0.9 mM, or 1.0 mM.

In some embodiments, the pharmaceutical composition comprises an LSD1 inhibitor that is RN-1 and a GSK3 Inhibitor that is CHIR99021. The RN-1 at a concentration of about 0.001 mM to 100,000 mM, 0.01 mM to 10,000 mM, about 0.1 mM to 1,000 mM, about 0.1 mM to 100 mM, about 0.001 mM to 0.01 mM, about 0.01 mM to 0.1 mM, about 0.1 mM to 1 mM about 1 mM to 10 mM, about 10 mM to 100 mM, about 100 mM to 1,000 mM, or about 1,000 mM to 10,000 mM. and the CHIR99021 at a concentration of about 0.001 mM to 10,000 mM, about 0.01 mM to 1,000 mM, about 0.1 mM to 100 mM, about 0.001 mM to 0.01 mM, about 0.01 mM to 0.1 mM, about 0.1 mM to 1 mM, about 1 mM to 10 mM, about 10 mM to 100 mM, about 100 mM to 1,000 mM, or about 1,000 mM to 10,000 mM.

In some embodiments, at a concentration of about 0.1 mM, 0.2 mM, 0.3 mM, 0.4 mM, 0.5 mM, 0.6 mM, 0.7 mM, 0.8 mM, 0.9 mM, 1 mM, 2 mM, 3 mM, 4 mM, 5 mM, 6 mM, 7 mM, 8 mM, 9 mM, or 10 mM and the CHIR99021 is at a concentration of about 1 mM, 2 mM, 3 mM, 4 mM, 5 mM, 6 mM, 7 mM, 8 mM, 9 mM, or 10 mM.

In some embodiments, the pharmaceutical composition comprises an LSD1 inhibitor that is GSK-2879552 and a GSK3 Inhibitor that is LY2090314 and a HDAC inhibitor that is VPA. The GSK-2879552 is at a concentration of about 0.001 μM to 1,000 mM, about 0.01 μM to 100,000 μM, about 0.1 μM to 10,000 μM, about 1 μM to 1,000 μM, about 1 μM to 10 μM, about 10 μM to 100 μM, about 100 μM to 1 mM, or about 1 mM to 10 mM. and the LY2090314 at a concentration of about 0.001 μM to 10 mM, about 0.01 μM to 1 mM, about 0.1 μM to 100 uM, about 0.001 μM to 0.01 μM, about 0.01 μM to 0.1 μM, about 0.1 μM to 1 μM, about 1 μM to 10 μM, about 10 μM to 100 μM, about 100 μM to 1 mM, or about 1 mM to 10 mM and the VPA at a concentration about 100 mM to 4,000 mM.

In some embodiments, the GSK-2879552 at a concentration of about 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, 10 mM, 12 mM, 14 mM, 16 mM, 18 mM, 20 mM, 25 mM, or about 30 mM and the LY2090314 the is at a concentration of about 1 μM, 5 μM, 10 μM, 15 μM, 20 μM or 40 μM and the VPA at a concentration about 100 mM to 4,000 mM.

In some embodiments, the pharmaceutical composition comprises an LSD1 inhibitor that is GSK-2879552 and a GSK3 Inhibitor that is that is a substituted 3-Imidazo[1,2-a]pyridin-3-yl-4-(1,2,3,4-tetrahydro-[1,4]diazepino-[6,7,1-hi]indol-7-yl)pyrrole-2,5-dione and a HDAC inhibitor that is VPA. The GSK-2879552 is at a concentration of about 0.001 μM to 1,000 mM, about 0.01 μM to 100,000 μM, about 0.1 μM to 10,000 μM, about 1 μM to 1,000 μM, about 1 μM to 10 μM, about 10 μM to 100 μM, about 100 μM to 1 mM, or about 1 mM to 10 mM. and the substituted 3-Imidazo[1,2-a]pyridin-3-yl-4-(1,2,3,4-tetrahydro-[1,4]diazepino-[6,7,1-hi]indol-7-yl)pyrrole-2,5-dione at a concentration of about 0.001 μM to 10 mM, about 0.01 μM to 1 mM, about 0.1 μM to 100 uM, about 0.001 μM to 0.01 μM, about 0.01 μM to 0.1 μM, about 0.1 μM to 1 μM, about 1 μM to 10 μM, about 10 μM to 100 μM, about 100 μM to 1 mM, or about 1 mM to 10 mM and the VPA at a concentration about 100 mM to 4,000 mM.

In some embodiments, the GSK-2879552 at a concentration of about 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, 10 mM, 12 mM, 14 mM, 16 mM, 18 mM, 20 mM, 25 mM, or about 30 mM and the substituted 3-Imidazo[1,2-a]pyridin-3-yl-4-(1,2,3,4-tetrahydro-[1,4]diazepino-[6,7,1-hi]indol-7-yl)pyrrole-2,5-dione, is at a concentration of about 1 μM, 5 μM, 10 μM, 15 μM, 20 μM, 50 μM, 100 μM, 250 μM or 500 μM and the VPA at a concentration about 100 mM to 4,000 mM.

In some embodiments, the pharmaceutical composition comprises an LSD1 inhibitor that is GSK-2879552 and a GSK3 Inhibitor that is GSK3-inhibitor XXII and a HDAC inhibitor that is VPA. The GSK-2879552 is at a concentration of about 0.001 μM to 1,000 mM, about 0.01 μM to 100,000 μM, about 0.1 μM to 10,000 μM, about 1 μM to 1,000 μM, about 1 μM to 10 μM, about 10 μM to 100 μM, about 100 μM to 1 mM, or about 1 mM to 10 mM. and the GSK3-inhibitor XXII, at a concentration of about of about 0.1 μM to 1,000 mM, about 1 μM to 100 mM, about 10 μM to 10 mM, about 0.1 μM to 1 μM, about 1 μM to 10 μM, about 10 μM to 100 μM, about 100 μM to 1 mM, about 1 mM to 10 mM, about 10 mM to 100 mM, or about 100 mM to 1000 mM and the VPA at a concentration about 100 mM to 4,000 mM.

In some embodiments, the GSK-2879552 at a concentration of about 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, 10 mM, 12 mM, 14 mM, 16 mM, 18 mM, 20 mM, 25 mM, or about 30 mM and the GSK3-inhibitor XXII is at a concentration of about 0.1 mM, 0.2 mM, 0.3 mM, 0.4 mM, 0.5 mM, 0.6 mM, 0.7 mM, 0.8 mM, 0.9 mM, or 1.0 mM. and the VPA at a concentration about 100 mM to 4,000 mM.

In some embodiments, the pharmaceutical composition comprises an LSD1 inhibitor that is GSK-2879552 and a GSK3 Inhibitor that is CHIR99021 and a HDAC inhibitor that is VPA. The GSK-2879552 is at a concentration of about 0.001 μM to 1,000 mM, about 0.01 μM to 100,000 μM, about 0.1 μM to 10,000 μM, about 1 μM to 1,000 μM, about 1 μM to 10 μM, about 10 μM to 100 μM, about 100 μM to 1 mM, or about 1 mM to 10 mM. and the CHIR99021 at a concentration of about 0.001 mM to 10,000 mM, about 0.01 mM to 1,000 mM, about 0.1 mM to 100 mM, about 0.001 mM to 0.01 mM, about 0.01 mM to 0.1 mM, about 0.1 mM to 1 mM, about 1 mM to 10 mM, about 10 mM to 100 mM, about 100 mM to 1,000 mM, or about 1,000 mM to 10,000 mM and the VPA at a concentration about 100 mM to 4,000 mM.

In some embodiments, the GSK-2879552 at a concentration of about 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, 10 mM, 12 mM, 14 mM, 16 mM, 18 mM, 20 mM, 25 mM, or about 30 mM and the CHIR99021 is at a concentration of about 1 mM, 2 mM, 3 mM, 4 mM, 5 mM, 6 mM, 7 mM, 8 mM, 9 mM, or 10 mM and the VPA at a concentration about 100 mM to 4,000 mM.

In some embodiments, the pharmaceutical composition comprises a LSD1 inhibitor that is ORY-1001 and a GSK3 Inhibitor that is LY2090314. The ORY-1001 at a concentration of about 0.001 mM to 100,000 mM, 0.01 mM to 10,000 mM, about 0.1 mM to 1,000 mM, about 0.1 mM to 100 mM, about 0.001 mM to 0.01 mM, about 0.01 mM to 0.1 mM, about 0.1 mM to 1 mM, about 1 mM to 10 mM, about 10 mM to 100 mM, about 100 mM to 1,000 mM, or about 1,000 mM to 10,000 mM. and the LY2090314 at a concentration of about 0.001 μM to 10 mM, about 0.01 μM to 1 mM, about 0.1 μM to 100 uM, about 0.001 μM to 0.01 μM, about 0.01 μM to 0.1 μM, about 0.1 μM to 1 μM, about 1 μM to 10 μM, about 10 μM to 100 μM, about 100 μM to 1 mM, or about 1 mM to 10 mM at a concentration of about 0.1 mM, 0.2 mM, 0.3 mM, 0.4 mM, 0.5 mM, 0.6 mM, 0.7 mM, 0.8 mM, 0.9 mM, 1 mM, 2 mM, 3 mM, 4 mM, 5 mM, 6 mM, 7 mM, 8 mM, 9 mM, or 10 mM and the LY2090314 the is at a concentration of about 1 μM, 5 μM, 10 μM, 15 μM, 20 μM, or 40 μM.

In some embodiments, the pharmaceutical composition comprises an LSD1 inhibitor that is ORY-1001 and a GSK3 Inhibitor that is that is a substituted 3-Imidazo[1,2-a]pyridin-3-yl-4-(1,2,3,4-tetrahydro-[1,4]diazepino-[6,7,1-hi]indol-7-yl)pyrrole-2,5-dione. The ORY-1001 at a concentration of about 0.001 mM to 100,000 mM, 0.01 mM to 10,000 mM, about 0.1 mM to 1,000 mM, about 0.1 mM to 100 mM, about 0.001 mM to 0.01 mM, about 0.01 mM to 0.1 mM, about 0.1 mM to 1 mM, about 1 mM to 10 mM, about 10 mM to 100 mM, about 100 mM to 1,000 mM, or about 1,000 mM to 10,000 mM. and the substituted 3-Imidazo[1,2-a]pyridin-3-yl-4-(1,2,3,4-tetrahydro-[1,4]diazepino-[6,7,1-hi]indol-7-yl)pyrrole-2,5-dione at a concentration of about 0.001 μM to 10 mM, about 0.01 μM to 1 mM, about 0.1 μM to 100 uM, about 0.001 μM to 0.01 μM, about 0.01 μM to 0.1 μM, about 0.1 μM to 1 μM, about 1 μM to 10 μM, about 10 μM to 100 μM, about 100 μM to 1 mM, or about 1 mM to 10 mM.

In some embodiments, at a concentration of about 0.1 mM, 0.2 mM, 0.3 mM, 0.4 mM, 0.5 mM, 0.6 mM, 0.7 mM, 0.8 mM, 0.9 mM, 1 mM, 2 mM, 3 mM, 4 mM, 5 mM, 6 mM, 7 mM, 8 mM, 9 mM, or 10 mM and the substituted 3-Imidazo[1,2-a]pyridin-3-yl-4-(1,2,3,4-tetrahydro-[1,4]diazepino-[6,7,1-hi]indol-7-yl)pyrrole-2,5-dione, is at a concentration of about 1 μM, 5 μM, 10 μM, 15 μM, 20 μM, 50 μM, 100 μM, 250 μM or 500 μM.

In some embodiments, the pharmaceutical composition comprises an LSD1 inhibitor that is ORY-1001 and a GSK3 Inhibitor that is GSK3-inhibitor XXII. The ORY-1001 at a concentration of about 0.001 mM to 100,000 mM, 0.01 mM to 10,000 mM, about 0.1 mM to 1,000 mM, about 0.1 mM to 100 mM, about 0.001 mM to 0.01 mM, about 0.01 mM to 0.1 mM, about 0.1 mM to 1 mM, about 1 mM to 10 mM, about 10 mM to 100 mM, about 100 mM to 1,000 mM, or about 1,000 mM to 10,000 mM. and the GSK3-inhibitor XXII, at a concentration of about of about 0.1 μM to 1,000 mM, about 1 μM to 100 mM, about 10 μM to 10 mM, about 0.1 μM to 1 μM, about 1 μM to 10 μM, about 10 μM to 100 μM, about 100 μM to 1 mM, about 1 mM to 10 mM, about 10 mM to 100 mM, or about 100 mM to 1000 mM.

In some embodiments, at a concentration of about 0.1 mM, 0.2 mM, 0.3 mM, 0.4 mM, 0.5 mM, 0.6 mM, 0.7 mM, 0.8 mM, 0.9 mM, 1 mM, 2 mM, 3 mM, 4 mM, 5 mM, 6 mM, 7 mM, 8 mM, 9 mM, or 10 mM. and the GSK3-inhibitor XXII is at a concentration of about 0.1 mM, 0.2 mM, 0.3 mM, 0.4 mM, 0.5 mM, 0.6 mM, 0.7 mM, 0.8 mM, 0.9 mM, or 1.0 mM.

In some embodiments, the pharmaceutical composition comprises an LSD1 inhibitor that is ORY-1001 and a GSK3 Inhibitor that is CHIR99021. The ORY-1001 at a concentration of about 0.001 mM to 100,000 mM, 0.01 mM to 10,000 mM, about 0.1 mM to 1,000 mM, about 0.1 mM to 100 mM, about 0.001 mM to 0.01 mM, about 0.01 mM to 0.1 mM, about 0.1 mM to 1 mM, about 1 mM to 10 mM, about 10 mM to 100 mM, about 100 mM to 1,000 mM, or about 1,000 mM to 10,000 mM, and the CHIR99021 at a concentration of about 0.001 mM to 10,000 mM, about 0.01 mM to 1,000 mM, about 0.1 mM to 100 mM, about 0.001 mM to 0.01 mM, about 0.01 mM to 0.1 mM, about 0.1 mM to 1 mM, about 1 mM to 10 mM, about 10 mM to 100 mM, about 100 mM to 1,000 mM, or about 1,000 mM to 10,000 mM.

In some embodiments, at a concentration of about 0.1 mM, 0.2 mM, 0.3 mM, 0.4 mM, 0.5 mM, 0.6 mM, 0.7 mM, 0.8 mM, 0.9 mM, 1 mM, 2 mM, 3 mM, 4 mM, 5 mM, 6 mM, 7 mM, 8 mM, 9 mM, or 10 mM and the CHIR99021 is at a concentration of about 1 mM, 2 mM, 3 mM, 4 mM, 5 mM, 6 mM, 7 mM, 8 mM, 9 mM, or 10 mM.

In some embodiments, the pharmaceutical composition comprises an LSD1 inhibitor that is ORY-1001 and a GSK3 Inhibitor that is AZD1080 and a HDAC inhibitor that is VPA. The ORY-1001 is at a concentration of about 0.001 μM to 1,000 mM, about 0.01 μM to 100,000 μM, about 0.1 μM to 10,000 μM, about 1 μM to 1,000 μM, about 1 μM to 10 μM, about 10 μM to 100 μM, about 100 μM to 1 mM, or about 1 mM to 10 mM. and the AZ1090 is at a concentration of about 0.001 mM to 10,000 mM, about 0.01 mM to 1,000 mM, about 0.1 mM to 100 mM, about 0.001 mM to 0.01 mM, about 0.01 mM to 0.1 mM, about 0.1 mM to 1 mM, about 1 mM to 10 mM, about 10 mM to 100 mM, about 100 mM to 1,000 mM, or about 1,000 mM to 10,000 mM and the VPA at a concentration about 100 mM to 4,000 mM.

In some embodiments, the ORY-1001 at a concentration of about 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, 10 mM, 12 mM, 14 mM, 16 mM, 18 mM, 20 mM, 25 mM, or about 30 mM and the AZ1090 is at a a concentration of about 1 mM, 2 mM, 3 mM, 4 mM, 5 mM, 6 mM, 7 mM, 8 mM, 9 mM, or 10 mM and the VPA at a concentration about 100 mM to 4,000 mM.

In some embodiments, the pharmaceutical composition comprises an LSD1 inhibitor that is ORY-1001 and a GSK3 Inhibitor that is LY2090314 and a HDAC inhibitor that is VPA. The ORY-1001 is at a concentration of about 0.001 μM to 1,000 mM, about 0.01 μM to 100,000 μM, about 0.1 μM to 10,000 μM, about 1 μM to 1,000 μM, about 1 μM to 10 μM, about 10 μM to 100 μM, about 100 μM to 1 mM, or about 1 mM to 10 mM. and the LY 2090314 at a concentration of about 0.001 μM to 10 mM, about 0.01 μM to 1 mM, about a 1 μM to 100 uM, about 0.001 μM to 0.01 μM, about 0.01 μM to 0.1 μM, about 0.1 μM to 1 μM, about 1 μM to 10 μM, about 10 μM to 100 μM, about 100 μM to 1 mM, or about 1 mM to 10 mM and the VPA at a concentration about 100 mM to 4,000 mM.

In some embodiments, the ORY-1001 at a concentration of about 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, 10 mM, 12 mM, 14 mM, 16 mM, 18 mM, 20 mM, 25 mM, or about 30 mM and the LY2090314 the is at a concentration of about 1 μM, 5 μM, 10 μM, 15 μM, 20 μM or 40 μM and the VPA at a concentration about 100 mM to 4,000 mM.

In some embodiments, the pharmaceutical composition comprises an LSD1 inhibitor that is ORY-1001 and a GSK3 Inhibitor that is that is a substituted 3-Imidazo[1,2-a]pyridin-3-yl-4-(1,2,3,4-tetrahydro-[1,4]diazepino-[6,7,1-hi]indol-7-yl)pyrrole-2,5-dione and a HDAC inhibitor that is VPA. The GSK-2879552 is at a concentration of about 0.001 μM to 1,000 mM, about 0.01 μM to 100,000 μM, about 0.1 μM to 10,000 μM, about 1 μM to 1,000 μM, about 1 μM to 10 μM, about 10 μM to 100 μM, about 100 μM to 1 mM, or about 1 mM to 10 mM. and the substituted 3-Imidazo[1,2-a]pyridin-3-yl-4-(1,2,3,4-tetrahydro-[1,4]diazepino-[6,7,1-hi]indol-7-yl)pyrrole-2,5-dione at a concentration of about 0.001 μM to 10 mM, about 0.01 μM to 1 mM, about 0.1 μM to 100 uM, about 0.001 μM to 0.01 μM, about 0.01 μM to 0.1 μM, about 0.1 μM to 1 μM, about 1 μM to 10 μM, about 10 μM to 100 μM, about 100 μM to 1 mM, or about 1 mM to 10 mM. and the VPA at a concentration about 100 mM to 4,000 mM.

In some embodiments, the ORY-1001 at a concentration of about 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, 10 mM, 12 mM, 14 mM, 16 mM, 18 mM, 20 mM, 25 mM, or about 30 mM and the substituted 3-Imidazo[1,2-a]pyridin-3-yl-4-(1,2,3,4-tetrahydro-[1,4]diazepino-[6,7,1-hi]indol-7-yl)pyrrole-2,5-dione, is at a concentration of about 1 μM, 5 μM, 10 μM, 15 μM, 20 μM, 50 μM, 100 μM, 250 μM or 500 μM and the VPA at a concentration about 100 mM to 4,000 mM.

In some embodiments, the pharmaceutical composition comprises an LSD1 inhibitor that is ORY-1001 and a GSK3 Inhibitor that is GSK3-inhibitor XXII and a HDAC inhibitor that is VPA. The ORY-1001 is at a concentration of about 0.001 μM to 1,000 mM, about 0.01 μM to 100,000 μM, about 0.1 μM to 10,000 μM, about 1 μM to 1,000 μM, about 1 μM to 10 μM, about 10 μM to 100 μM, about 100 μM to 1 mM, or about 1 mM to 10 mM. and the GSK3-inhibitor XXII, at a concentration of about of about 0.1 μM to 1,000 mM, about 1 μM to 100 mM, about 10 μM to 10 mM, about 0.1 μM to 1 μM, about 1 μM to 10 μM, about 10 μM to 100 μM, about 100 μM to 1 mM, about 1 mM to 10 mM, about 10 mM to 100 mM, or about 100 mM to 1000 mM and the VPA at a concentration about 100 mM to 4,000 mM.

In some embodiments, the ORY-1001 at a concentration of about 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, 10 mM, 12 mM, 14 mM, 16 mM, 18 mM, 20 mM, 25 mM, or about 30 mM and the GSK3-inhibitor XXII is at a concentration of about 0.1 mM, 0.2 mM, 0.3 mM, 0.4 mM, 0.5 mM, 0.6 mM, 0.7 mM, 0.8 mM, 0.9 mM, or 1.0 mM. and the VPA at a concentration about 100 mM to 4,000 mM.

In some embodiments, the pharmaceutical composition comprises an LSD1 inhibitor that is ORY-1001 and a GSK3 Inhibitor that is CHIR99021 and a HDAC inhibitor that is VPA. The GSK-2879552 is at a concentration of about 0.001 μM to 1,000 mM, about 0.01 μM to 100,000 μM, about 0.1 μM to 10,000 μM, about 1 μM to 1,000 μM, about 1 μM to 10 μM, about 10 μM to 100 μM, about 100 μM to 1 mM, or about 1 mM to 10 mM. and the CHIR99021 at a concentration of about 0.001 mM to 10,000 mM, about 0.01 mM to 1,000 mM, about 0.1 mM to 100 mM, about 0.001 mM to 0.01 mM, about 0.01 mM to 0.1 mM, about 0.1 mM to 1 mM, about 1 mM to 10 mM, about 10 mM to 100 mM, about 100 mM to 1,000 mM, or about 1,000 mM to 10,000 mM and the VPA at a concentration about 100 mM to 4,000 mM.

In some embodiments, the ORY-1001 at a concentration of about 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, 10 mM, 12 mM, 14 mM, 16 mM, 18 mM, 20 mM, 25 mM, or about 30 mM and the CHIR99021 is at a concentration of about 1 mM, 2 mM, 3 mM, 4 mM, 5 mM, 6 mM, 7 mM, 8 mM, 9 mM, or 10 mM and the VPA at a concentration about 100 mM to 4,000 mM.

In some embodiments, the pharmaceutical composition comprises a LSD1 inhibitor that is RN-1 and a GSK3 Inhibitor that is LY2090314. The RN-1 at a concentration of about 0.001 mM to 100,000 mM, 0.01 mM to 10,000 mM, about 0.1 mM to 1,000 mM, about 0.1 mM to 100 mM, about 0.001 mM to 0.01 mM, about 0.01 mM to 0.1 mM, about 0.1 mM to 1 mM about 1 mM to 10 mM, about 10 mM to 100 mM, about 100 mM to 1,000 mM, or about 1,000 mM to 10,000 mM. and the LY2090314 at a concentration of about 0.001 μM to 10 mM, about 0.01 μM to 1 mM, about 0.1 μM to 100 uM, about 0.001 μM to 0.01 μM, about 0.01 μM to 0.1 μM, about 0.1 μM to 1 μM, about 1 μM to 10 μM, about 10 μM to 100 μM, about 100 μM to 1 mM, or about 1 mM to 10 mM at a concentration of about 0.1 mM, 0.2 mM, 0.3 mM, 0.4 mM, 0.5 mM, 0.6 mM, 0.7 mM, 0.8 mM, 0.9 mM, 1 mM, 2 mM, 3 mM, 4 mM, 5 mM, 6 mM, 7 mM, 8 mM, 9 mM, or 10 mM and the LY2090314 the is at a concentration of about 1 μM, 5 μM, 10 μM, 15 μM, 20 μM, or 40 μM.

In some embodiments, the pharmaceutical composition comprises an LSD1 inhibitor that is RN-1 and a GSK3 Inhibitor that is that is a substituted 3-Imidazo[1,2-a]pyridin-3-yl-4-(1,2,3,4-tetrahydro-[1,4]diazepino-[6,7,1-hi]indol-7-yl)pyrrole-2,5-dione. The RN-1 at a concentration of about 0.001 mM to 100,000 mM, 0.01 mM to 10,000 mM, about 0.1 mM to 1,000 mM, about 0.1 mM to 100 mM, about 0.001 mM to 0.01 mM, about 0.01 mM to 0.1 mM, about 0.1 mM to 1 mM, about 1 mM to 10 mM, about 10 mM to 100 mM, about 100 mM to 1,000 mM, or about 1,000 mM to 10,000 mM. and the substituted 3-Imidazo[1,2-a]pyridin-3-yl-4-(1,2,3,4-tetrahydro-[1,4]diazepino-[6,7,1-hi]indol-7-yl)pyrrole-2,5-dione at a concentration of about 0.001 μM to 10 mM, about 0.01 μM to 1 mM, about 0.1 μM to 100 uM, about 0.001 μM to 0.01 μM, about 0.01 μM to 0.1 μM, about 0.1 μM to 1 μM, about 1 μM to 10 μM, about 10 μM to 100 μM, about 100 μM to 1 mM, or about 1 mM to 10 mM.

In some embodiments, at a concentration of about 0.1 mM, 0.2 mM, 0.3 mM, 0.4 mM, 0.5 mM, 0.6 mM, 0.7 mM, 0.8 mM, 0.9 mM, 1 mM, 2 mM, 3 mM, 4 mM, 5 mM, 6 mM, 7 mM, 8 mM, 9 mM, or 10 mM and the substituted 3-Imidazo[1,2-a]pyridin-3-yl-4-(1,2,3,4-tetrahydro-[1,4]diazepino-[6,7,1-hi]indol-7-yl)pyrrole-2,5-dione, is at a concentration of about 1 μM, 5 μM, 10 μM, 15 μM, 20 μM, 50 μM, 100 μM, 250 μM or 500 μM.

In some embodiments, the pharmaceutical composition comprises an LSD1 inhibitor that is RN-1 and a GSK3 Inhibitor that is GSK3-inhibitor XXII. The RN-1 at a concentration of about 0.001 mM to 100,000 mM, 0.01 mM to 10,000 mM, about 0.1 mM to 1,000 mM, about 0.1 mM to 100 mM, about 0.001 mM to 0.01 mM, about 0.01 mM to 0.1 mM, about 0.1 mM to 1 mM, about 1 mM to 10 mM, about 10 mM to 100 mM, about 100 mM to 1,000 mM, or about 1,000 mM to 10,000 mM. and the GSK3-inhibitor XXII, at a concentration of about of about 0.1 μM to 1,000 mM, about 1 μM to 100 mM, about 10 μM to 10 mM, about 0.1 μM to 1 μM, about 1 μM to 10 μM, about 10 μM to 100 μM, about 100 μM to 1 mM, about 1 mM to 10 mM, about 10 mM to 100 mM, or about 100 mM to 1000 mM.

In some embodiments, at a concentration of about 0.1 mM, 0.2 mM, 0.3 mM, 0.4 mM, 0.5 mM, 0.6 mM, 0.7 mM, 0.8 mM, 0.9 mM, 1 mM, 2 mM, 3 mM, 4 mM, 5 mM, 6 mM, mM, 8 mM, 9 mM, or 10 mM. and the GSK3-inhibitor XXII is at a concentration of about 0.1 mM, 0.2 mM, 0.3 mM, 0.4 mM, 0.5 mM, 0.6 mM, 0.7 mM, 0.8 mM, 0.9 mM, or 1.0 mM.

In some embodiments, the pharmaceutical composition comprises an LSD1 inhibitor that is RN-1 and a GSK3 Inhibitor that is CHIR99021. The RN-1 at a concentration of about 0.001 mM to 100,000 mM, 0.01 mM to 10,000 mM, about 0.1 mM to 1,000 mM, about 0.1 mM to 100 mM, about 0.001 mM to 0.01 mM, about 0.01 mM to 0.1 mM, about 0.1 mM to 1 mM, about 1 mM to 10 mM, about 10 mM to 100 mM, about 100 mM to 1,000 mM, or about 1,000 mM to 10,000 mM. and the CHIR99021 at a concentration of about 0.001 mM to 10,000 mM, about 0.01 mM to 1,000 mM, about 0.1 mM to 100 mM, about 0.001 mM to 0.01 mM, about 0.01 mM to 0.1 mM, about 0.1 mM to 1 mM, about 1 mM to 10 mM, about 10 mM to 100 mM, about 100 mM to 1,000 mM, or about 1,000 mM to 10,000 mM.

In some embodiments, at a concentration of about 0.1 mM, 0.2 mM, 0.3 mM, 0.4 mM, 0.5 mM, 0.6 mM, 0.7 mM, 0.8 mM, 0.9 mM, 1 mM, 2 mM, 3 mM, 4 mM, 5 mM, 6 mM, 7 mM, 8 mM, 9 mM, or 10 mM and the CHIR99021 is at a concentration of about 1 mM, 2 mM, 3 mM, 4 mM, 5 mM, 6 mM, 7 mM, 8 mM, 9 mM, or 10 mM.

In some embodiments, the pharmaceutical composition comprises an LSD1 inhibitor that is RN-1 and a GSK3 Inhibitor that is AZD1080 and a HDAC inhibitor that is VPA. The RN-1 is at a concentration of about 0.001 μM to 1,000 mM, about 0.01 μM to 100,000 μM, about 0.1 μM to 10,000 μM, about 1 μM to 1,000 μM, about 1 μM to 10 μM, about 10 μM to 100 μM, about 100 μM to 1 mM, or about 1 mM to 10 mM. and the AZ1090 is at a concentration of about 0.001 mM to 10,000 mM, about 0.01 mM to 1,000 mM, about 0.1 mM to 100 mM, about 0.001 mM to 0.01 mM, about 0.01 mM to 0.1 mM, about 0.1 mM to 1 mM, about 1 mM to 10 mM, about 10 mM to 100 mM, about 100 mM to 1,000 mM, or about 1,000 mM to 10,000 mM and the VPA at a concentration about 100 mM to 4,000 mM.

In some embodiments, the RN-1 at a concentration of about 0.1 μM, 0.2 nM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, 10 mM, 12 mM, 14 mM, 16 mM, 18 mM, 20 mM, 25 mM, or about 30 mM and the AZ1090 is at a a concentration of about 1 mM, 2 mM, 3 mM, 4 mM, 5 mM, 6 mM, 7 mM, 8 mM, 9 mM, or 10 mM and the VPA at a concentration about 100 mM to 4,000 mM.

In some embodiments, the pharmaceutical composition comprises an LSD1 inhibitor that is RN-1 and a GSK3 Inhibitor that is LY2090314 and a HDAC inhibitor that is VPA. The RN-1 is at a concentration of about 0.001 μM to 1,000 mM, about 0.01 μM to 100,000 μM, about 0.1 μM to 10,000 μM, about 1 μM to 1,000 μM, about 1 μM to 10 μM, about 10 μM to 100 μM, about 100 μM to 1 mM, or about 1 mM to 10 mM. and the LY2090314 at a concentration of about 0.001 μM to 10 mM, about 0.01 μM to 1 mM, about 0.1 μM to 100 uM, about 0.001 μM to 0.01 μM, about 0.01 μM to 0.1 μM, about 0.1 μM to 1 μM, about 1 μM to 10 μM, about 10 μM to 100 μM, about 100 μM to 1 mM, or about 1 mM to 10 mM and the VPA at a concentration about 100 mM to 4,000 mM.

In some embodiments, the RN-1 at a concentration of about 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, 10 mM, 12 mM, 14 mM, 16 mM, 18 mM, 20 mM, 25 mM, or about 30 mM and the LY2090314 the is at a concentration of about 1 μM, 5 μM, 10 μM, 15 μM, 20 μM or 40 μM and the VPA at a concentration about 100 mM to 4,000 mM.

In some embodiments, the pharmaceutical composition comprises an LSD1 inhibitor that is RN-1 and a GSK3 Inhibitor that is that is a substituted 3-Imidazo[1,2-a]pyridin-3-yl-4-(1,2,3,4-tetrahydro-[1,4]diazepino-[6,7,1-hi]indol-7-yl)pyrrole-2,5-dione and a HDAC inhibitor that is VPA. The GSK-2879552 is at a concentration of about 0.001 μM to 1,000 mM, about 0.01 μM to 100,000 μM, about 0.1 μM to 10,000 μM, about 1 μM to 1,000 μM, about 1 μM to 10 μM, about 10 μM to 100 μM, about 100 μM to 1 mM, or about 1 mM to 10 mM. and the substituted 3-Imidazo[1,2-a]pyridin-3-yl-4-(1,2,3,4-tetrahydro-[1,4]diazepino-[6,7,1-hi]indol-7-yl)pyrrole-2,5-dione at a concentration of about 0.001 μM to 10 mM, about 0.01 μM to 1 mM, about 0.1 μM to 100 uM, about 0.001 μM to 0.01 μM, about 0.01 μM to 0.1 μM, about 0.1 μM to 1 μM, about 1 μM to 10 μM, about 10 μM to 100 μM, about 100 μM to 1 mM, or about 1 mM to 10 mM and the VPA at a concentration about 100 mM to 4,000 mM.

In some embodiments, the RN-1 at a concentration of about 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, 10 mM, 12 mM, 14 mM, 16 mM, 18 mM, 20 mM, 25 mM, or about 30 mM and the substituted 3-Imidazo[1,2-a]pyridin-3-yl-4-(1,2,3,4-tetrahydro-[1,4]diazepino-[6,7,1-hi]indol-7-yl)pyrrole-2,5-dione, is at a concentration of about 1 μM, 5 μM, 10 μM, 15 μM, 20 μM, 50 μM, 100 μM, 250 μM or 500 μM and the VPA at a concentration about 100 mM to 4,000 mM.

In some embodiments, the pharmaceutical composition comprises an LSD1 inhibitor that is RN-1 and a GSK3 Inhibitor that is GSK3-inhibitor XXII and a HDAC inhibitor that is VPA. The RN-1 is at a concentration of about 0.001 μM to 1,000 mM, about 0.01 μM to 100,000 μM, about 0.1 μM to 10,000 μM, about 1 μM to 1,000 μM, about 1 μM to 10 μM, about 10 μM to 100 μM, about 100 μM to 1 mM, or about 1 mM to 10 mM. and the GSK3-inhibitor XXII, at a concentration of about of about 0.1 μM to 1,000 mM, about 1 μM to 100 mM, about 10 μM to 10 mM, about 0.1 μM to 1 μM, about 1 μM to 10 μM, about 10 μM to 100 μM, about 100 μM to 1 mM, about 1 mM to 10 mM, about 10 mM to 100 mM, or about 100 mM to 1000 mM and the VPA at a concentration about 100 mM to 4,000 mM.

In some embodiments, the RN-1 at a concentration of about 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, 10 mM, 12 mM, 14 mM, 16 mM, 18 mM, 20 mM, 25 mM, or about 30 mM and the GSK3-inhibitor XXII is at a concentration of about 0.1 mM, 0.2 mM, 0.3 mM, 0.4 mM, 0.5 mM, 0.6 mM, 0.7 mM, 0.8 mM, 0.9 mM, or 1.0 mM. and the VPA at a concentration about 100 mM to 4,000 mM.

In some embodiments, the pharmaceutical composition comprises an LSD1 inhibitor that is RN-1 and a GSK3 Inhibitor that is CHIR99021 and a HDAC inhibitor that is VPA. The GSK-2879552 is at a concentration of about 0.001 μM to 1,000 mM, about 0.01 μM to 100,000 μM, about 0.1 μM to 10,000 μM, about 1 μM to 1,000 μM, about 1 μM to 10 μM, about 10 μM to 100 μM, about 100 μM to 1 mM, or about 1 mM to 10 mM. and the CHIR99021 at a concentration of about 0.001 mM to 10,000 mM, about 0.01 mM to 1,000 mM, about 0.1 mM to 100 mM, about 0.001 mM to 0.01 mM, about 0.01 mM to 0.1 mM, about 0.1 mM to 1 mM, about 1 mM to 10 mM, about 10 mM to 100 mM, about 100 mM to 1,000 mM, or about 1,000 mM to 10,000 mM and the VPA at a concentration about 100 mM to 4,000 mM.

In some embodiments, the RN-1 at a concentration of about 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, 10 mM, 12 mM, 14 mM, 16 mM, 18 mM, 20 mM, 25 mM, or about 30 mM and the CHIR99021 is at a concentration of about 1 mM, 2 mM, 3 mM, 4 mM, 5 mM, 6 mM, 7 mM, 8 mM, 9 mM, or 10 mM and the VPA at a concentration about 100 mM to 4,000 mM.

In some embodiments, the pharmaceutical composition comprises an EZH2 inhibitor that is CPI-1205 and a GSK3 Inhibitor that is AZD1080. The CPI-1205 is at a concentration of about 0.001 μM to 1,000 mM, about 0.01 μM to 100,000 μM, about 0.1 μM to 10,000 μM, about 1 μM to 1,000 μM, about 1 μM to 10 μM, about 10 μM to 100 μM, about 100 μM to 1 mM, or about 1 mM to 10 mM. and the AZ1090 is at a concentration of about 0.001 mM to 10,000 mM, about 0.01 mM to 1,000 mM, about 0.1 mM to 100 mM, about 0.001 mM to 0.01 mM, about 0.01 mM to 0.1 mM, about 0.1 mM to 1 mM, about 1 mM to 10 mM, about 10 mM to 100 mM, about 100 mM to 1,000 mM, or about 1,000 mM to 10,000 mM.

In some embodiments, the CPI-1205 at a concentration of about 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, 10 mM, 12 mM, 14 mM, 16 mM, 18 mM, 20 mM, 25 mM, or about 30 mM and the AZ1090 is at a a concentration of about 1 mM, 2 mM, 3 mM, 4 mM, 5 mM, 6 mM, 7 mM, 8 mM, 9 mM, or 10 mM.

In some embodiments, the pharmaceutical composition comprises an EZH2 inhibitor that is CPI-1205 and a GSK3 Inhibitor that is LY2090314. The CPI-1205 is at a concentration of about 0.001 μM to 1,000 mM, about 0.01 μM to 100,000 μM, about 0.1 μM to 10,000 μM, about 1 μM to 1,000 μM, about 1 μM to 10 μM, about 10 μM to 100 μM, about 100 μM to 1 mM, or about 1 mM to 10 mM. and the LY2090314 at a concentration of about 0.001 μM to 10 mM, about 0.01 μM to 1 mM, about 0.1 μM to 100 uM, about 0.001 μM, to 0.01 μM, about 0.01 μM to 0.1 μM, about 0.1 μM to 1 μM, about 1 μM to 10 μM, about 10 μM to 100 μM, about 100 μM to 1 mM, or about 1 mM to 10 mM.

In some embodiments, the CPI-1205 at a concentration of about 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, 10 mM, 12 mM, 14 mM, 16 mM, 18 mM, 20 mM, 25 mM, or about 30 mM and the LY2090314 the is at a concentration of about 1 μM, 5 μM, 10 μM, 15 μM, 20 μM, or 40 μM.

In some embodiments, the pharmaceutical composition comprises an EZH2 inhibitor that is CTI-1205 and a GSK3 Inhibitor that is that is a substituted 3-Imidazo[1,2-a]pyridin-3-yl-4-(1,2,3,4-tetrahydro-[1,4]diazepino-[6,7,1-hi]indol-7-yl)pyrrole-2,5-dione. The GPI-1205 is at a concentration of about 0.001 μM to 1,000 mM, about 0.01 μM to 100,000 μM, about 0.1 μM to 10,000 μM, about 1 μM to 1,000 μM, about 1 μM to 10 μM, about 10 μM to 100 μM, about 100 μM to 1 mM, or about 1 mM to 10 mM. and the substituted 3-Imidazo[1,2-a]pyridin-3-yl-4-(1,2,3,4-tetrahydro-[1,4]diazepino-[6,7,1-hi]indol-7-yl)pyrrole-2,5-dione at a concentration of about 0.001 μM to 10 mM, about 0.01 μM to 1 mM, about 0.1 μM to 100 uM, about 0.001 μM to 0.01 μM, about 0.01 μM to 0.1 μM, about 0.1 μM to 1 μM, about 1 μM to 10 μM, about 10 μM to 100 μM, about 100 μM to 1 mM, or about 1 mM to 10 mM.

In some embodiments, the CPI-1205 at a concentration of about 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM or about 10 mM and the substituted 3-Imidazo[1,2-a]pyridin-3-yl-4-(1,2,3,4-tetrahydro-[1,4]diazepino-[6,7,1-hi]indol-7-yl)pyrrole-2,5-dione, is at a concentration of about 1 μM, 5 μM, 10 μM, 15 μM, 20 μM, 50 μM, 100 μM, 250 μM or 500 μM.

In some embodiments, the pharmaceutical composition comprises an EZH2 inhibitor that is CPI-1205 and a GSK3 Inhibitor that is GSK3-inhibitor XXII. The CPI-1205 is at a concentration of about 0.001 μM to 1,000 mM, about 0.01 μM to 100,000 μM, about 0.1 μM to 10,000 μM, about 1 μM to 1,000 μM, about 1 μM to 10 μM, about 10 μM to 100 μM, about 100 μM to 1 mM, or about 1 mM to 10 mM. and the GSK3-inhibitor XXII, at a concentration of about of about 0.1 μM to 1,000 mM, about 1 μM to 100 mM, about 10 μM to 10 mM, about 0.1 μM to 1 μM, about 1 μM to 10 μM, about 10 μM to 100 μM, about 100 μM to 1 mM, about 1 mM to 10 mM, about 10 mM to 100 mM, or about 100 mM to 1000 mM.

In some embodiments, the CPI-1205 at a concentration of about 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, 10 mM, 12 mM, 14 mM, 16 mM, 18 mM, 20 mM, 25 mM, or about 30 mM and the GSK3-inhibitor XXII is at a concentration of about 0.1 mM, 0.2 mM, 0.3 mM, 0.4 mM, 0.5 mM, 0.6 mM, 0.7 mM, 0.8 mM, 0.9 mM, or 1.0 mM.

In some embodiments, the pharmaceutical composition comprises an EZH2 inhibitor that is CPI-1205 and a GSK3 Inhibitor that is CHIR99021. The CPI-1205 is at a concentration of about 0.001 μM to 1,000 mM, about 0.01 μM to 100,000 μM, about 0.1 μM to 10,000 μM, about 1 μM to 1,000 μM, about 1 μM to 10 μM, about 10 μM to 100 μM, about 100 μM to 1 mM, or about 1 mM to 10 mM. and the CHIR99021 at a concentration of about 0.001 mM to 10,000 mM, about 0.01 mM to 1,000 mM, about 0.1 mM to 100 mM, about 0.001 mM to 0.01 mM, about 0.01 mM to 0.1 mM, about 0.1 mM to 1 mM, about 1 mM to 10 mM, about 10 mM to 100 mM, about 100 mM to 1,000 mM, or about 1,000 mM to 10,000 mM.

In some embodiments, the CPI-1205 at a concentration of about 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, 10 mM, 12 mM, 14 mM, 16 mM, 18 mM, 20 mM, 25 mM, or about 30 mM and the CHIR99021 is at a concentration of about 1 mM, 2 mM, 3 mM, 4 mM, 5 mM, 6 mM, 7 mM, 8 mM, 9 mM, or 10 mM.

In some embodiments, the pharmaceutical composition comprises an EZH2 inhibitor that is CPI-169 and a GSK3 Inhibitor that is AZD1080. The CPI-169 is at a concentration of about 0.001 μM to 1,000 mM, about 0.01 μM to 100,000 μM, about 0.1 μM to 10,000 μM, about 1 μM to 1,000 μM, about 1 μM to 10 μM, about 10 μM to 100 μM, about 100 μM to 1 mM, or about 1 mM to 10 mM, and the AZ1090 is at a concentration of about 0.001 mM to 10,000 mM, about 0.01 mM to 1,000 mM, about 0.1 mM to 100 mM, about 0.001 mM to 0.01 mM, about 0.01 mM to 0.1 mM, about 0.1 mM to 1 mM, about 1 mM to 10 mM, about 10 mM to 100 mM, about 100 mM to 1,000 mM, or about 1,000 mM, to 10,000 mM.

In some embodiments, the CPI-169 at a concentration of about 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, 10 mM, 12 mM, 14 mM, 16 mM, 18 mM, 20 mM, 25 mM, or about 30 mM and the AZ1090 is at a a concentration of about 1 mM, 2 mM, 3 mM, 4 mM, 5 mM, 6 mM, 7 mM, 8 mM, 9 mM, or 10 mM.

In some embodiments, the pharmaceutical composition comprises an EZH2 inhibitor that is CPI-169 and a GSK3 Inhibitor that is LY2090314. The CPI-169 is at a concentration of about 0.001 μM to 1,000 mM, about 0.01 μM to 100,000 μM, about 0.1 μM to 10,000 μM, about 1 μM to 1,000 μM, about 1 μM to 10 μM, about 10 μM to 100 μM, about 100 μM to 1 mM, or about 1 mM to 10 mM. and the LY2090314 at a concentration of about 0.001 μM to 10 mM, about 0.01 μM to 1 mM, about 0.1 μM to 100 uM, about 0.001 μM to 0.01 μM, about 0.01 μM to 0.1 μM, about 0.1 μM to 1 μM, about 1 μM to 10 μM, about 10 μM to 100 μM, about 100 μM to 1 mM, or about 1 mM to 10 mM.

In some embodiments, the CPI-169 at a concentration of about 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, 10 mM, 12 mM, 14 mM, 16 mM, 18 mM, 20 mM, 25 mM, or about 30 mM and the LY2090314 the is at a concentration of about 1 μM, 5 μM, 10 μM, 15 μM, 20 μM, or 40 μM.

In some embodiments, the pharmaceutical composition comprises an EZH2 inhibitor that is CPI-169 and a GSK3 Inhibitor that is that is a substituted 3-Imidazo[1,2-a]pyridin-3-yl-4-(1,2,3,4-tetrahydro-[1,4]diazepino-[6,7,1-hi]indol-7-yl)pyrrole-2,5-dione. The CPI-169 is at a concentration of about 0.001 μM to 1,000 mM, about 0.01 μM to 100,000 μM, about 0.1 μM to 10,000 μM, about 1 μM to 1,000 μM, about 1 μM to 10 μM, about 10 μM to 100 μM, about 100 μM to 1 mM, or about 1 mM to 10 mM. and the substituted 3-Imidazo[1,2-a]pyridin-3-yl-4-(1,2,3,4-tetrahydro-[1,4]diazepino-[6,7,1-hi]indol-7-yl)pyrrole-2,5-dione at a concentration of about 0.001 μM to 10 mM, about 0.01 μM to 1 mM, about 0.1 μM to 100 uM, about 0.001 μM to 0.01 μM, about 0.01 μM to 0.1 μM, about 0.1 μM to 1 μM, about 1 μM to 10 μM, about 10 μM to 100 μM, about 100 μM to 1 mM, or about 1 mM to 10 mM.

In some embodiments, the CPI-169 at a concentration of about 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM or about 10 mM and the substituted 3-Imidazo[1,2-a]pyridin-3-yl-4-(1,2,3,4-tetrahydro-[1,4]diazepino-[6,7,1-hi]indol-7-yl)pyrrole-2,5-dione, is at a concentration of about 1 μM, 5 μM, 10 μM, 15 μM, 20 μM, 50 μM, 100 μM, 250 μM or 500 μM.

In some embodiments, the pharmaceutical composition comprises an EZH2 inhibitor that is CPI-169 and a GSK3 Inhibitor that is GSK3-inhibitor XXII. The CPI-169 is at a concentration of about 0.001 μM to 1,000 mM, about 0.01 μM to 100,000 μM, about 0.1 μM to 10,000 μM, about 1 μM to 1,000 μM, about 1 μM to 10 μM, about 10 μM to 100 μM, about 100 μM to 1 mM, or about 1 mM to 10 mM, and the GSK3-inhibitor XXII, at a concentration of about 0.1 μM to 1,000 mM, about 1 μM to 100 mM, about 10 μM to 10 mM about 0.1 μM to 1 μM, about 1 μM to 10 μM, about 10 μM to 100 μM, about 100 μM to 1 mM, about 1 mM to 10 mM, about 10 mM to 100 mM, or about 100 mM to 1000 mM.

In some embodiments, the CPI-169 at a concentration of about 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, 10 mM, 12 mM, 14 mM, 16 mM, 18 mM, 20 mM, 25 mM, or about 30 mM, and the GSK3-inhibitor XXII is at a concentration of about 0.1 mM, 0.2 mM, 0.3 mM, 0.4 mM, 0.5 mM, 0.6 mM, 0.7 mM, 0.8 mM, 0.9 mM, or 1.0 mM.

In some embodiments, the pharmaceutical composition comprises an EZH2 inhibitor that is CPI-169 and a GSK3 Inhibitor that is CHIR99021. The CPI-169 is at a concentration of about 0.001 μM to 1,000 mM, about 0.01 μM to 100,000 μM, about 0.1 μM to 10,000 μM, about 1 μM to 1,000 μM, about 1 μM to 10 μM, about 10 μM to 100 μM, about 100 μM to 1 mM, or about 1 mM to 10 mM. and the CHIR99021 at a concentration of about 0.001 mM to 10,000 mM, about 0.01 mM to 1,000 mM, about 0.1 mM to 100 mM, about 0.001 mM to 0.01 mM, about 0.01 mM to 0.1 mM, about 0.1 mM to 1 mM, about 1 mM to 10 mM, about 10 mM to 100 mM, about 100 mM to 1,000 mM, or about 1,000 mM to 10,000 mM.

In some embodiments, the CPI-169 at a concentration of about 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, 10 mM, 12 mM, 14 mM, 16 mM, 18 mM, 20 mM, 25 mM, or about 30 mM and the CHIR99021 is at a concentration of about 1 mM, 2 mM, 3 mM, 4 mM, 5 mM, 6 mM, 7 mM, 8 mM, 9 mM, or 10 mM.

In some embodiments, the pharmaceutical composition comprises an EZH2 inhibitor that is EL1 and a GSK3 Inhibitor that is AZD1080. The EL1 is at a concentration of about 0.001 μM to 1,000 mM, about 0.01 μM to 100,000 μM, about 0.1 μM to 10,000 μM, about 1 μM to 1,000 μM, about 1 μM to 10 μM, about 10 μM to 100 μM, about 100 μM to 1 mM, or about 1 mM to 10 mM, and the AZ1090 is at a concentration of about 0.001 mM to 10,000 mM, about 0.01 mM to 1,000 mM, about 0.1 mM to 100 mM, about 0.001 mM to 0.01 mM, about 0.01 mM to 0.1 mM, about 0.1 mM to 1 mM, about 1 mM to 10 mM, about 10 mM to 100 mM, about 100 mM to 1,000 mM, or about 1,000 mM to 10,000 mM.

In some embodiments, the EL1 at a concentration of about 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, 10 mM, 12 mM, 14 mM, 16 mM, 18 mM, 20 mM, 25 mM, or about 30 mM and the AZ1090 is at a a concentration of about 1 mM, 2 mM, 3 mM, 4 mM, 5 mM, 6 mM, 7 mM, 8 mM, 9 mM, or 10 mM.

In some embodiments, the pharmaceutical composition comprises an EZH2 inhibitor that is EL1 and a GSK3 Inhibitor that is LY2090314. The EL1 is at a concentration of about 0.001 μM to 1,000 mM, about 0.01 μM to 100,000 μM, about 0.1 μM to 10,000 μM, about 1 μM to 1,000 μM, about 1 μM to 10 μM, about 10 μM to 100 μM, about 100 μM to 1 mM, or about 1 mM to 10 mM. and the LY2090314 at a concentration of about 0.001 μM to 10 mM, about 0.01 μM to 1 mM, about 0.1 μM to 100 uM, about 0.001 μM to 0.01 μM, about 0.01 μM to 0.1 μM, about 0.1 μM to 1 μM, about 1 μM to 10 μM, about 10 μM to 100 μM, about 100 μM to 1 mM, or about 1 mM to 10 mM.

In some embodiments, the EL1 at a concentration of about 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, 10 mM, 12 mM, 14 mM, 16 mM, 18 nM, 20 mM, 25 mM, or about 30 mM and the LY2090314 the is at a concentration of about 1 μM, 5 μM, 10 μM, 15 μM, 20 μM, or 40 μM.

In some embodiments, the pharmaceutical composition comprises an EZH2 inhibitor that is EL1 and a GSK3 Inhibitor that is that is a substituted 3-Imidazo[1,2-a]pyridin-3-yl-4-(1,2,3,4-tetrahydro-[1,4]diazepino-[6,7,1-hi]indol-7-yl)pyrrole-2,5-dione. The EL1 is at a concentration of about 0.001 μM to 1,000 mM, about 0.01 μM to 100,000 μM, about 0.1 μM to 10,000 μM, about 1 μM to 1,000 μM, about 1 μM to 10 μM, about 10 μM to 100 μM, about 100 μM to 1 mM, or about 1 mM to 10 mM. and the substituted 3-Imidazo[1,2-a]pyridin-3-yl-4-(1,2,3,4-tetrahydro-[1,4]diazepino-[6,7,1-hi]indol-7-yl)pyrrole-2,5-dione at a concentration of about 0.001 μM to 10 mM, about 001 μM to 1 mM, about 0.1 μM to 100 uM, about 0.001 μM to 0.01 μM, about 0.01 μM to 0.1 μM, about 0.1 μM to 1 μM, about 1 μM to 10 μM, about 10 μM to 100 μM, about 100 μM to 1 mM, or about 1 mM to 10 mM.

In some embodiments, the EL1 at a concentration of about 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM or about 10 mM and the substituted 3-Imidazo[1,2-a]pyridin-3-yl-4-(1,2,3,4-tetrahydro-[1,4]diazepino-[6,7,1-hi]indol-7-yl)pyrrole-2,5-dione, is at a concentration of about 1 μM, 5 μM, 10 μM, 15 μM, 20 μM, 50 μM, 100 μM, 250 μM or 500 μM.

In some embodiments, the pharmaceutical composition comprises an EZH2 inhibitor that is EL1 and a GSK3 Inhibitor that is GSK3-inhibitor XXII. The EL1 is at a concentration of about 0.001 μM to 1,000 mM, about 0.01 μM to 100,000 μM, about 0.1 μM to 10,000 μM, about 1 μM to 1,000 μM, about 1 μM to 10 μM, about 10 μM to 100 μM, about 100 μM to 1 mM, or about 1 mM to 10 mM. and the GSK3-inhibitor XXII, at a concentration of about of about 0.1 μM to 1,000 mM, about 1 μM to 100 mM, about 10 μM to 10 mM, about 0.1 μM to 1 μM, about 1 μM to 10 μM, about 10 μM to 100 μM, about 100 μM to 1 mM, about 1 mM to 10 mM about 10 mM to 100 mM, or about 100 mM to 1000 mM.

In some embodiments, the EL1 at a concentration of about 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, 10 mM, 12 mM, 14 mM, 16 mM, 18 mM, 20 mM, 25 mM, or about 30 mM and the GSK3-inhibitor XXII is at a concentration of about 0.1 mM, 0.2 mM, 0.3 mM, 0.4 mM, 0.5 mM, 0.6 mM, 0.7 mM, 0.8 mM, 0.9 mM, or 1.0 mM.

In some embodiments, the pharmaceutical composition comprises an EZH2 inhibitor that is EL1 and a GSK3 Inhibitor that is CHIR99021. The EL1 is at a concentration of about 0.001 μM to 1,000 mM, about 0.01 μM to 100,000 μM, about 0.1 μM to 10,000 μM, about 1 μM to 1,000 μM, about 1 μM to 10 μM, about 10 μM to 100 μM, about 100 μM to 1 mM, or about 1 mM to 10 mM. and the CHIR99021 at a concentration of about 0.001 mM to 10,000 mM, about 0.01 mM to 1,000 mM, about 0.1 mM to 100 mM, about 0.001 mM to 0.01 mM, about 0.01 mM to 0.1 mM, about 0.1 mM to 1 mM, about 1 mM to 10 mM, about 10 mM to 100 mM, about 100 mM to 1,000 mM, or about 1,000 mM to 10,000 mM.

In some embodiments, the EL1 at a concentration of about 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, 10 mM, 12 mM, 14 mM, 16 mM, 18 mM, 20 mM, 25 mM, or about 30 mM and the CHIR99021 is at a concentration of about 1 mM, 2 mM, 3 mM, 4 mM, 5 mM, 6 mM, 7 mM, 8 mM, 9 mM, or 10 mM.

In some embodiments, the pharmaceutical composition comprises an EZH2 inhibitor that is PF-06821497 and a GSK3 Inhibitor that is AZD1080. The PF-06821497 is at a concentration of about 0.001 μM to 1,000 mM, about 0.01 μM to 100,000 μM, about 0.1 μM to 10,000 μM, about 1 μM to 1,000 μM, about 1 μM to 10 μM, about 10 μM to 100 μM, about 100 μM to 1 mM, or about 1 mM to 10 mM. and the AZ1090 is at a concentration of about 0.001 mM to 10,000 mM, about 0.01 mM to 1,000 mM, about 0.1 mM to 100 mM, about 0.001 mM to 0.01 mM, about 0.01 mM to 0.1 mM, about 0.1 mM to 1 mM, about 1 mM to 10 mM, about 10 mM to 100 mM, about 100 mM to 1,000 mM, or about 1,000 mM to 10,000 mM.

In some embodiments, the PF-06821497 at a concentration of about 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, 10 mM, 12 mM, 14 mM, 16 mM, 18 mM, 20 mM, 25 mM, or about 30 mM and the AZ1090 is at a a concentration of about 1 mM, 2 mM, 3 mM, 4 mM, 5 mM, 6 mM, 7 mM, 8 mM, 9 mM, or 10 mM.

In some embodiments, the pharmaceutical composition comprises an EZH2 inhibitor that is PF-06821497 and a GSK3 Inhibitor that is LY2090314. The PF-06821497 is at a concentration of about 0.001 μM to 1,000 mM, about 0.01 μM to 100,000 μM, about 0.1 μM to 10,000 μM, about 1 μM to 1,000 μM, about 1 μM to 10 μM, about 10 μM to 100 μM, about 100 μM to 1 mM, or about 1 mM to 10 mM. and the LY2090314 at a concentration of about 0.001 μM to 10 mM, about 0.01 μM to 1 mM, about 0.1 μM to 100 uM, about 0.001 μM to 0.01 μM, about 0.01 μM to 0.1 μM, about 0.1 μM to 1 μM, about 1 μM to 10 μM, about 10 μM to 100 μM, about 100 μM to 1 mM, or about 1 mM to 10 mM.

In some embodiments, the PF-06821497 at a concentration of about 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, 10 mM, 12 mM, 14 mM, 16 mM, 18 mM, 20 mM, 25 mM, or about 30 mM and the LY2090314 the is at a concentration of about 1 μM, 5 μM, 10 μM, 15 μM, 20 μM, or 40 μM.

In some embodiments, the pharmaceutical composition comprises a EZH2 inhibitor that is PF-06821497 and a GSK3 Inhibitor that is that is a substituted 3-Imidazo[1,2-a]pyridin-3-yl-4-(1,2,3,4-tetrahydro-[1,4]diazepino-[6,7,1-hi]indol-7-yl)pyrrole-2,5-dione. The PF-06821497 is at a concentration of about 0.001 μM to 1,000 mM, about 0.01 μM to 100,000 μM, about 0.1 μM to 10,000 μM, about 1 μM to 1,000 μM, about 1 μM to 10 μM, about 10 μM to 100 μM, about 100 μM to 1 mM, or about 1 mM to 10 mM. and the substituted 3-Imidazo[1,2-a]pyridin-3-yl-4-(1,2,3,4-tetrahydro-[1,4]diazepino-[6,7,1-hi]indol-7-yl)pyrrole-2,5-dione at a concentration of about 0.001 μM to 10 mM, about 0.01 μM to 1 mM, about 0.1μM to 100 uM, about 0.001 μM to 0.01 μM, about 0.01 μM to 0.1 μM, about 0.1 μM to 1 μM, about 1 μM to 10 μM, about 10 μM to 100 μM, about 100 μM to 1 mM, or about 1 mM to 10 mM.

In some embodiments, the PF-06821497 at a concentration of about 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM or about 10 mM and the substituted 3-Imidazo[1,2-a]pyridin-3-yl-4-(1,2,3,4-tetrahydro-[1,4]diazepino-[6,7,1-hi]indol-7-yl)pyrrole-2,5-dione, is at a concentration of about 1 μM, 5 μM, 10 μM, 15 μM, 20 μM, 50 μM, 100 μM, 250 μM or 500 μM.

In some embodiments, the pharmaceutical composition comprises an EZH2 inhibitor that is PF-06821497 and a GSK3 Inhibitor that is GSK3-inhibitor XXII. The PF-06821497 is at a concentration of about 0.001 μM to 1,000 mM, about 0.01 μM to 100,000 μM, about 0.1 μM to 10,000 μM, about 1 μM to 1,000 μM, about 1 μM to 10 μM, about 10 μM to 100 μM, about 100 μM to 1 mM, or about 1 mM to 10 mM. and the GSK3-inhibitor XXII, at a concentration of about of about 0.1 μM to 1,000 mM, about 1 μM to 100 mM, about 10 μM to 10 mM, about 0.1 μM to 1 μM, about 1 μM to 10 μM, about 10 μM to 100 μM, about 100 μM to 1 mM, about 1 mM to 10 mM, about 10 mM to 100 mM, or about 100 mM to 1000 mM.

In some embodiments, the PF-06821497 at a concentration of about 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, 10 mM, 12 mM, 14 mM, 16 mM, 18 mM, 20 mM, 25 mM, or about 30 mM and the GSK3-inhibitor XXII is at a concentration of about 0.1 mM, 0.2 mM, 0.3 mM, 0.4 mM, 0.5 mM, 0.6 mM, 0.7 mM, 0.8 mM, 0.9 mM, or 1.0 mM.

In some embodiments, the pharmaceutical composition comprises an EZH2 inhibitor that is PF-06821497 and a GSK3 Inhibitor that is CHIR99021. The PF-06821497 is at a concentration of about 0.001 μM to 1,000 mM, about 0.01 μM to 100,000 μM, about 0.1 μM to 10,000 μM, about 1 μM to 1,000 μM, about 1 μM to 10 μM, about 10 μM to 100 μM, about 100 μM to 1 mM, or about 1 mM to 10 mM. and the CHIR99021 at a concentration of about 0.001 mM to 10,000 mM, about 0.01 mM to 1,000 mM, about 0.1 mM to 100 mM, about 0.001 mM to 0.01 mM, about 0.01 mM to 0.1 mM, about 0.1 mM to 1 mM, about 1 mM to 10 mM, about 10 mM to 100 mM, about 100 mM to 1,000 mM, or about 1,000 mM to 10,000 mM.

In some embodiments, the PF-06821497 at a concentration of about 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, 10 mM, 12 mM, 14 mM, 16 mM, 18 mM, 20 mM, 25 mM, or about 30 mM and the CHIR99021 is at a concentration of about 1 mM, 2 mM, 3 mM, 4 mM, 5 mM, 6 mM, 7 mM, 8 mM, 9 mM, or 10 mM.

In some embodiments, the pharmaceutical composition comprises a EZH2 inhibitor that is tazemetostat and a GSK3 Inhibitor that is AZD1080. The tazemetostat is at a concentration of about 0.001 μM to 1,000 mM, about 0.01 μM to 100,000 μM, about 0.1 μM to 10,000 μM, about 1 μM to 1,000 μM, about 1 μM to 10 μM, about 10 μM to 100 μM, about 100 μM to 1 mM, or about 1 mM to 10 mM. and the AZ1090 is at a concentration of about 0.001 mM to 10,000 mM, about 0.01 mM to 1,000 mM, about 0.1 mM to 100 mM, about 0.001 mM to 0.01 mM, about 0.01 mM to 0.1 mM, about 0.1 mM to 1 mM, about 1 mM to 10 mM, about 10 mM to 100 mM, about 100 mM to 1,000 mM, or about 1,000 mM to 10,000 mM.

In some embodiments, the tazemetostat at a concentration of about 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, 10 mM, 12 mM, 14 mM, 16 mM, 18 mM, 20 mM, 25 mM, or about 30 mM and the AZ1090 is at a a concentration of about 1 mM, 2 mM, 3 mM, 4 mM, 5 mM, 6 mM, 7 mM, 8 mM, 9 mM, or 10 mM.

In some embodiments, the pharmaceutical composition comprises an EZH2 inhibitor that is tazemetostat and a GSK3 Inhibitor that is LY2090314. The tazemetostat is at a concentration of about 0.001 μM to 1,000 mM, about 0.01 μM to 100,000 μM, about 0.1 μM to 10,000 μM, about 1 μM to 1,000 μM, about 1 μM to 10 μM, about 10 μM to 100 μM, about 100 μM to 1 mM, or about 1 mM to 10 mM. and the LY2090314 at a concentration of about 0.001 μM to 10 mM, about 0.01 μM to 1 mM, about 0.1 μM to 100 uM, about 0.001 μM to 0.01 μM, about 0.01 μM to 0.1 μM, about 0.1 μM to 1 μM, about 1 μM to 10 μM, about 10 μM to 100 μM, about 100 μM to 1 mM, or about 1 mM to 10 mM.

In some embodiments, the tazemetostat at a concentration of about 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, 10 mM, 12 mM, 14 mM, 16 mM, 18 mM, 20 mM, 25 mM, or about 30 mM and the LY2090314 the is at a concentration of about 1 μM, 5 μM, 10 μM, 15 μM, 20 μM, or 40 μM.

In some embodiments, the pharmaceutical composition comprises a EZH2 inhibitor that is tazemetostat and a GSK3 Inhibitor that is that is a substituted 3-Imidazo[1,2-a]pyridin-3-yl-4-(1,2,3,4-tetrahydro-[1,4]diazepino-[6,7,1-hi]indol-7-yl)pyrrole-2,5-dione. The tazemetostat is at a concentration of about 0.001 μM to 1,000 mM, about 0.01 μM to 100,000 μM, about 0.1 μM to 10,000 μM, about 1 μM to 1,000 μM, about 1 μM to 10 μM, about 10 μM to 100 μM, about 100 μM to 1 mM, or about 1 mM to 10 mM. and the substituted 3-Imidazo[1,2-a]pyridin-3-yl-4-(1,2,3,4-tetrahydro-[1,4]diazepino-[6,7,1-hi]indol-7-yl)pyrrole-2,5-dione at a concentration of about 0.001 μM to 10 mM, about 0.01 μM to 1 mM, about 0.1 μM to 100 uM, about 0.001 μM to 0.01 μM, about 0.01 μM to 0.1 μM, about 0.1 μM to 1 μM, about 1 μM to 10 μM, about 10 μM to 100 μM, about 100 μM to 1 mM, or about 1 mM to 10 mM.

In some embodiments, the tazemetostat at a concentration of about 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM or about 10 mM and the substituted 3-Imidazo[1,2-a]pyridin-3-yl-4-(1,2,3,4-tetrahydro-[1,4]diazepino-[6,7,1-hi]indol-7-yl)pyrrole-2,5-dione, is at a concentration of about 1 μM, 5 μM, 10 μM, 15 μM, 20 μM, 50 μM, 100 μM, 250 μM or 500 μM.

In some embodiments, the pharmaceutical composition comprises an EZH2 inhibitor that is tazemetostat and a GSK3 Inhibitor that is GSK3-inhibitor XXII. The tazemetostat is at a concentration of about 0.001 μM to 1,000 mM, about 0.01 μM to 100,000 μM, about 0.1 μM to 10,000 μM, about 1 μM to 1,000 μM, about 1 μM to 10 μM, about 10 μM to 100 μM, about 100 μM to 1 mM, or about 1 mM to 10 mM. and the GSK3-inhibitor XXII, at a concentration of about of about 0.1 μM to 1,000 mM, about 1 μM to 100 mM, about 10 μM to 10 mM, about 0.1 μM to 1 μM, about 1 μM to 10 μM, about 10 μM to 100 μM, about 100 μM to 1 mM, about 1 mM to 10 mM, about 10 mM to 100 mM, or about 100 mM to 1000 mM.

In some embodiments, the tazemetostat at a concentration of about 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, 10 mM, 12 mM, 14 mM, 16 mM, 18 mM, 20 mM, 25 mM, or about 30 mM and the GSK3-inhibitor XXII is at a concentration of about 0.1 mM, 0.2 mM, 0.3 mM, 0.4 mM, 0.5 mM, 0.6 mM, 0.7 mM, 0.8 mM, 0.9 mM, or 1.0 mM.

In some embodiments, the pharmaceutical composition comprises an EZH2 inhibitor that is tazemetostat and a GSK3 Inhibitor that is CHTR99021. The tazemetostat is at a concentration of about 0.001 μM to 1,000 mM, about 0.01 μM to 100,000 μM, about 0.1 μM to 10,000 μM, about 1 μM to 1,000 μM, about 1 μM to 10 μM, about 10 μM to 100 μM, about 100 μM to 1 mM, or about 1 mM to 10 mM. and the CHIR99021 at a concentration of about 0.001 mM to 10,000 mM, about 0.01 mM to 1,000 mM, about 0.1 mM to 100 mM, about 0.001 mM to 0.01 mM, about 0.01 mM to 0.1 mM, about 0.1 mM to 1 mM, about 1 mM to 10 mM, about 10 mM to 100 mM, about 100 mM to 1,000 mM, or about 1,000 mM to 10,000 mM.

In some embodiments, the tazemetostat at a concentration of about 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, 10 mM, 12 mM, 14 mM, 16 mM, 18 mM, 20 mM, 25 mM, or about 30 mM and the CHIR99021 is at a concentration of about 1 mM, 2 mM, 3 mM, 4 mM, 5 mM, 6 mM, 7 mM, 8 mM, 9 mM, or 10 mM.

In some embodiments, the pharmaceutical composition comprises an EZH2 inhibitor that is valemetostat and a GSK3 Inhibitor that is AZD1080. The valemetostat is at a concentration of about 0.001 μM to 1,000 mM, about 0.01 μM to 100,000 μM, about 0.1 μM to 10,000 μM, about 1 μM to 1,000 μM, about 1 μM to 10 μM, about 10 μM to 100 μM, about 100 μM to 1 mM, or about 1 mM to 10 mM. and the AZ1090 is at a concentration of about 0.001 mM to 10,000 mM, about 0.01 mM to 1,000 mM, about 0.1 mM to 100 mM, about 0.001 mM to 0.01 mM, about 0.01 mM to 0.1 mM, about 0.1 mM to 1 mM, about 1 mM to 10 mM, about 10 mM to 100 mM, about 100 mM to 1,000 mM, or about 1,000 mM to 10,000 mM.

In some embodiments, the valemetostat at a concentration of about 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, 10 mM, 12 mM, 14 mM, 16 mM, 18 mM, 20 mM, 25 mM, or about 30 mM and the AZ1090 is at a a concentration of about 1 mM, 2 mM, 3 mM, 4 mM, 5 mM, 6 mM, 7 mM, 8 mM, 9 mM, or 10 mM.

In some embodiments, the pharmaceutical composition comprises an EZH2 inhibitor that is valemetostat and a GSK3 Inhibitor that is LY2090314. The valemetostat is at a concentration of about 0.001 μM to 1,000 mM, about 0.01 μM to 100,000 μM, about 0.1 μM to 10,000 μM, about 1 μM to 1,000 μM, about 1 μM to 10 μM, about 10 μM to 100 μM, about 100 μM to 1 mM, or about 1 mM to 10 mM, and the LY2090314 at a concentration of about 0.001 μM to 10 mM, about 0.01 μM to 1 mM, about 0.1 μM to 100 uM, about 0.001 μM to 0.01 μM, about 0.01 μM to 0.1 μM, about 0.1 μM to 1 μM, about 1 μM to 10 μM, about 10 μM to 100 μM, about 100 μM to 1 mM, or about 1 mM to 10 mM.

In some embodiments, the valemetostat at a concentration of about 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, 10 mM, 12 mM, 14 mM, 16 mM, 18 mM, 20 mM, 25 mM, or about 30 mM and the LY2090314 the is at a concentration of about 1 μM, 5 μM, 10 μM, 15 μM, 20 μM, or 40 μM.

In some embodiments, the pharmaceutical composition comprises an EZH2 inhibitor that is valemetostat and a GSK3 Inhibitor that is that is a substituted 3-Imidazo[1,2-a]pyridin-3-yl-4-(1,2,3,4-tetrahydro-[1,4]diazepino-[6,7,1-hi]indol-7-yl)pyrrole-2,5-dione. The valemetostat is at a concentration of about 0.001 μM to 1,000 mM, about 0.01 μM to 100,000 μM, about 0.1 μM to 10,000 μM, about 1 μM to 1,000 μM, about 1 μM to 10 μM, about 10 μM to 100 μM, about 100 μM to 1 mM, or about 1 mM to 10 mM. and the substituted 3-Imidazo[1,2-a]pyridin-3-yl-4-(1,2,3,4-tetrahydro-[1,4]diazepino-[6,7,1-hi]indol-7-yl)pyrrole-2,5-dione at a concentration of about 0.001 μM to 10 mM, about 0.01 μM to 1 mM, about 0.1 μM to 100 uM, about 0.001 μM to 0.01 μM, about 0.01 μM to 0.1 μM, about 0.1 μM to 1 μM, about 1 μM to 10 μM, about 10 μM to 100 μM, about 100 μM to 1 mM, or about 1 mM to 10 mM.

In some embodiments, the valemetostat at a concentration of about 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM or about 10 mM and the substituted 3-Imidazo[1,2-a]pyridin-3-yl-4-(1,2,3,4-tetrahydro-[1,4]diazepino-[6,7,1-hi]indol-7-yl)pyrrole-2,5-dione, is at a concentration of about 1 μM, 5 μM, 10 μM, 15 μM, 20 μM, 50 μM, 100 μM, 250 μM or 500 μM.

In some embodiments, the pharmaceutical composition comprises an EZH2 inhibitor that is valemetostat and a GSK3 Inhibitor that is GSK3-inhibitor XXII. The valemetostat is at a concentration of about 0.001 μM to 1,000 mM, about 0.01 μM to 100,000 μM, about 0.1 μM to 10,000 μM, about 1 μM to 1,000 μM, about 1 μM to 10 μM, about 10 μM to 100 μM, about 100 μM to 1 mM, or about 1 mM to 10 mM. and the GSK3-inhibitor XXII, at a concentration of about of about 0.1 μM to 1,000 mM, about 1 μM to 100 mM, about 10 μM to 10 mM, about 0.1 μM to 1 μM, about 1 μM to 10 μM, about 10 μM to 100 μM, about 100 μM to 1 mM, about 1 mM to 10 mM, about 10 mM to 100 mM, or about 100 mM to 1000 mM.

In some embodiments, the valemetostat at a concentration of about 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, 10 mM, 12 mM, 14 mM, 16 mM, 18 mM, 20 mM, 25 mM, or about 30 mM, and the GSK3-inhibitor XXII is at a concentration of about 0.1 mM, 0.2 mM, 0.3 mM, 0.4 mM, 0.5 mM, 0.6 mM, 0.7 mM, 0.8 mM, 0.9 mM, or 1.0 mM.

In some embodiments, the pharmaceutical composition comprises an EZH2 inhibitor that is valemetostat and a GSK3 Inhibitor that is CHIR99021. The valemetostat is at a concentration of about 0.001 μM to 1,000 mM, about 0.01 μM to 100,000 μM, about 0.1 μM to 10,000 μM, about 1 μM to 1,000 μM, about 1 μM to 10 μM, about 10 μM to 100 μM, about 100 μM to 1 mM, or about 1 mM to 10 mM. and the CHIR99021 at a concentration of about 0.001 mM to 10,000 mM, about 0.01 mM to 1,000 mM, about 0.1 mM to 100 mM, about 0.001 mM to 0.01 mM, about 0.01 mM to 0.1 mM, about 0.1 mM to 1 mM, about 1 mM to 10 mM, about 10 mM to 100 mM, about 100 mM to 1,000 mM, or about 1,000 mM to 10,000 mM.

In some embodiments, the valemetostat at a concentration of about 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, 10 mM, 12 mM, 14 mM, 16 mM, 18 mM, 20 mM, 25 mM, or about 30 mM and the CHIR99021 is at a concentration of about 1 mM, 2 mM, 3 mM, 4 mM, 5 mM, 6 mM, 7 mM, 8 mM, 9 mM, or 10 mM.

In some embodiments, at a concentration of about 0.1 mM, 0.2 mM, 0.3 mM, 0.4 mM, 0.5 mM, 0.6 mM, 0.7 mM, 0.8 mM, 0.9 mM, 1 mM, 2 mM, 3 mM, 4 mM, 5 mM, 6 mM, 7 mM, 8 mM, 9 mM, or 10 mM and the CHIR99021 is at a concentration of about 1 mM, 2 mM, 3 mM, 4 mM, 5 mM, 6 mM, 7 mM, 8 mM, 9 mM, or 10 mM.

In some embodiments, the pharmaceutical composition comprises an EZH2 inhibitor that is CPI-360 and a GSK3 inhibitor that is AZD1080. The CPI-360 is at a concentration of about 0.001 μM to 1,000 mM, about 0.01 μM to 100,000 μM, about 0.1 μM to 10,000 μM, about 1 μM to 1,000 μM, about 1 μM to 10 μM, about 10 μM to 100 μM, about 100 μM to 1 mM, or about 1 mM to 10 mM, and the AZ1090 is at a concentration of about 0.001 mM to 10,000 mM, about 0.01 mM to 1,000 mM, about 0.1 mM to 100 mM, about 0.001 mM to 0.01 mM, about 0.01 mM to 0.1 mM, about 0.1 mM to 1 mM, about 1 mM to 10 mM, about 10 mM to 100 mM, about 100 mM to 1,000 mM, or about 1,000 mM to 10,000 mM.

In some embodiments, the CPI-360 at a concentration of about 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, 10 mM, 12 mM, 14 mM, 16 mM, 18 mM, 20 mM, 25 mM, or about 30 mM and the AZ1090 is at a a concentration of about 1 mM, 2 mM, 3 mM, 4 mM, 5 mM, 6 mM, 7 mM, 8 mM, 9 mM, or 10 mM.

In some embodiments, the pharmaceutical composition comprises an EZH2 inhibitor that is CPI-360 and a GSK3 Inhibitor that is LY2090314. The CPI-360 is at a concentration of about 0.001 μM to 1,000 mM, about 0.01 μM to 100,000 μM, about 0.1 μM to 10,000 μM, about 1 μM to 1,000 μM, about 1 μM to 10 μM, about 10 μM to 100 μM, about 100 μM to 1 mM, or about 1 mM to 10 mM. and the LY2090314 at a concentration of about 0.001 μM to 10 mM, about 0.01 μM to 1 mM, about 0.1 μM to 100 uM about 0.001 μM to 0.01 μM, about 0.01 μM to 0.1 μM, about 0.1 μM to 1 μM, about 1 μM to 10 μM, about 10 μM to 100 μM, about 100 μM to 1 mM, or about 1 mM to 10 mM.

In some embodiments, the CPI-360 at a concentration of about 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, 10 mM, 12 mM, 14 mM, 16 mM, 18 mM, 20 mM, 25 mM, or about 30 mM and the LY2090314 the is at a concentration of about 1 μM, 5 μM, 10 μM, 15 μM, 20 μM, or 40 μM.

In some embodiments, the pharmaceutical composition comprises an EZH2 inhibitor that is CPI-360 and a GSK3 Inhibitor that is that is a substituted 3-Imidazo[1,2-a]pyridin-3-yl-4-(1,2,3,4-tetrahydro-[1,4]diazepino-[6,7,1-hi]indol-7-yl)pyrrole-2,5-dione. The CPI-360 is at a concentration of about 0.001 μM to 1,000 mM, about 0.01 μM to 100,000 μM, about 0.1 μM to 10,000 μM, about 1 μM to 1,000 μM, about 1 μM to 10 μM, about 10 μM to 100 μM, about 100 μM to 1 mM, or about 1 mM to 10 mM, and the substituted 3-Imidazo[1,2-a]pyridin-3-yl-4-(1,2,3,4-tetrahydro-[1,4]diazepino-[6,7,1-hi]indol-7-yl)pyrrole-2,5-dione at a concentration of about 0.001 μM to 10 mM, about 0.01 μM to 1 mM, about 0.1 μM to 100 uM, about 0.001 μM to 0.01 μM, about 0.01 μM to 0.1 μM, about 0.1 μM to 1 μM, about 1 μM to 10 μM, about 10 μM to 100 μM, about 100 μM to 1 mM, or about 1 mM to 10 mM.

In some embodiments, the CPI-360 at a concentration of about 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM or about 10 mM and the substituted 3-Imidazo[1,2-a]pyridin-3-yl-4-(1,2,3,4-tetrahydro-[1,4]diazepino-[6,7,1-hi]indol-7-yl)pyrrole-2,5-dione, is at a concentration of about 1 μM, 5 μM, 10 μM, 15 μM, 20 μM, 50 μM, 100 μM, 250 μM or 500 μM.

In some embodiments, the pharmaceutical composition comprises an EZH2 inhibitor that is CPI-360 and a GSK3 Inhibitor that is GSK3-inhibitor XXII. The CPI-360 is at a concentration of about 0.001 μM to 1,000 mM, about 0.01 μM to 100,000 μM, about 0.1 μM to 10,000 μM, about 1 μM to 1,000 μM, about 1 μM to 10 μM, about 10 μM to 100 μM, about 100 μM to 1 mM, or about 1 mM to 10 mM. and the GSK3-inhibitor XXII, at a concentration of about of about 0.1 μM to 1,000 mM, about 1 μM to 100 mM, about 10 μM to 10 mM, about 0.1 μM to 1 μM, about 1 μM to 10 μM, about 10 μM to 100 μM, about 100 μM to 1 mM, about 1 mM to 10 mM, about 10 mM to 100 mM, or about 100 mM to 1000 mM.

In some embodiments, the CPI-360 at a concentration of about 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, 10 mM, 12 mM, 14 mM, 16 mM, 18 mM, 20 mM, 25 mM, or about 30 mM and the GSK3-inhibitor XVII is at a concentration of about 0.1 mM, 0.2 mM, 0.3 mM, 0.4 mM, 0.5 mM, 0.6 mM, 0.7 mM, 0.8 mM, 0.9 mM, or 1.0 mM.

In some embodiments, the pharmaceutical composition comprises an EZH2 inhibitor that is CPI-360 and a GSK3 Inhibitor that is CHIR99021. The CPI-360 is at a concentration of about 0.001 μM to 1,000 mM, about 0.01 μM to 100,000 μM, about 0.1 μM to 10,000 μM, about 1 μM to 1,000 μM, about 1 μM to 10 μM, about 10 μM to 100 μM, about 100 μM to 1 mM, or about 1 mM to 10 mM. and the CHIR99021 at a concentration of about 0.001 mM to 10,000 mM, about 0.01 mM to 1,000 mM, about 0.1 mM to 100 mM, about 0.001 mM to 0.01 mM, about 0.01 mM to 0.1 mM, about 0.1 mM to 1 mM, about 1 mM to 10 mM, about 10 mM to 100 mM, about 100 mM to 1,000 mM, or about 1,000 mM to 10,000 mM.

In some embodiments, the CPI-360 at a concentration of about 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, 10 mM, 12 mM, 14 mM, 16 mM, 18 mM, 20 mM, 25 mM or about 30 mM and the CHIR99021 is at a concentration of about 1 mM, 2 mM, 3 mM, 4 mM, 5 mM, 6 mM, 7 mM, 8 mM, 9 mM, or 10 mM.

In some embodiments, the pharmaceutical composition comprises an EZH2 inhibitor that is EPZ011989 and a GSK3 Inhibitor that is AZD1080. The EPZ011989 is at a concentration of about 0.001 μM to 1,000 mM, about 0.01 μM to 100,000 μM, about 0.1 μM to 10,000 μM, about 1 μM to 1,000 μM, about 1 μM to 10 μM, about 10 μM to 100 μM, about 100 μM to 1 mM, or about 1 mM to 10 mM. and the AZ1090 is at a concentration of about 0.001 mM to 10,000 mM, about 0.01 mM to 1,000 mM, about 0.1 mM to 100 mM, about 0.001 mM to 0.01 mM, about 0.01 mM to 0.1 mM, about 0.1 mM to 1 mM, about 1 mM to 10 mM, about 10 mM to 100 mM, about 100 mM to 1,000 mM, or about 1,000 mM to 10,000 mM.

In some embodiments, the EPZ011989 at a concentration of about 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, 10 mM, 12 mM, 14 mM, 16 mM, 18 mM, 20 mM, 25 mM, or about 30 mM and the AZ1090 is at a a concentration of about 1 mM, 2 mM, 3 mM, 4 mM, 5 mM, 6 mM, 7 mM, 8 mM, 9 mM, or 10 mM.

In some embodiments, the pharmaceutical composition comprises an EZH2 inhibitor that is EPZ011989 and a GSK3 Inhibitor that is LY2090314. The EPZ011989 is at a concentration of about 0.001 μM, to 1,000 mM, about 0.01 μM, to 100,000 μM, about 0.1 μM to 10,000 μM, about 1 μM to 1,000 μM, about 1 μM to 10 μM, about 10 μM to 100 μM, about 100 μM to 1 mM, or about 1 mM to 10 mM and the LY2090314 at a concentration of about 0.001 μM to 10 mM, about 0.01 μM to 1 mM, about 0.1 μM to 100 uM, about 0.001 μM to 0.01 μM, about 0.01 μM to 0.1 μM, about 0.1 μM to 1 μM, about 1 μM to 10 μM, about 10 μM to 100 μM, about 100 μM to 1 mM, or about 1 mM to 10 mM.

In some embodiments, the EPZ011989 at a concentration of about 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, 10 mM, 12 mM, 14 mM, 16 mM, 18 mM, 20 mM, 25 mM, or about 30 mM and the LY2090314 the is at a concentration of about 1 μM, 5 μM, 10 μM, 15 μM, 20 μM, or 40 μM.

In some embodiments, the pharmaceutical composition comprises an EZH2 inhibitor that is EPZ011989 and a GSK3 Inhibitor that is that is a substituted 3-Imidazo[1,2-a]pyridin-3-yl-4-(1,2,3,4-tetrahydro-[1,4]diazepino-[6,7,1-hi]indol-7-yl)pyrrole-2,5-dione. The EPZ011989 is at a concentration of about 0.001 μM to 1,000 mM, about 0.01 μM to 100,000 μM, about 0.1 μM to 10,000 μM, about 1 μM to 1,000 μM, about 1 μM to 10 μM, about 10 μM to 100 μM, about 100 μM to 1 mM, or about 1 mM to 10 mM, and the substituted 3-Imidazo[1,2-a]pyridin-3-yl-4-(1,2,3,4-tetrahydro-[1,4]diazepino-[6,7,1-hi]indol-7-yl)pyrrole-2,5-dione at a concentration of about 0.001 μM to 10 mM, about 0.01 μM to 1 mM, about 0.1 μM to 100 uM, about 0.001 μM to 0.01 μM, about 0.01 μM to 0.1 μM, about 0.1 μM to 1 μM, about 1 μM to 10 μM, about 10 μM to 100 μM, about 100 μM to 1 mM, or about 1 mM to 10 mM.

In some embodiments, the EPZ011989 at a concentration of about 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM or about 10 mM and the substituted 3-Imidazo[1,2-a]pyridin-3-yl-4-(1,2,3,4-tetrahydro-[1,4]diazepino-[6,7,1-hi]indol-7-yl)pyrrole-2,5-dione, is at a concentration of about 1 μM, 5 μM, 10 μM, 15 μM, 20 μM, 50 μM, 100 μM, 250 μM or 500 μM.

In some embodiments, the pharmaceutical composition comprises an EZH2 inhibitor that is EPZ011989 and a GSK3 Inhibitor that is GSK3-inhibitor XXII. The EPZ011989 is at a concentration of about 0.001 μM to 1,000 mM, about 0.01 μM to 100,000 μM, about 0.1 μM to 10,000 μM, about 1 μM to 1,000 μM, about 1 μM to 10 μM, about 10 μM to 100 μM, about 100 μM to 1 mM, or about 1 mM to 10 mM. and the GSK3-inhibitor XXII, at a concentration of about of about 0.1 μM to 1,000 mM, about 1 μM to 100 mM, about 10 μM to 10 mM, about 0.1 μM to 1 μM, about 1 μM to 10 μM, about 10 μM to 100 μM, about 100 μM to 1 mM, about 1 mM to 10 mM, about 10 mM to 100 mM, or about 100 mM to 1000 mM.

In some embodiments, the EPZ011989 at a concentration of about 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, 10 mM, 12 mM, 14 mM, 16 mM, 18 mM, 20 mM, 25 mM, or about 30 mM and the GSK3-inhibitor XXII is at a concentration of about 0.1 mM, 0.2 mM, 0.3 mM, 0.4 mM, 0.5 mM, 0.6 mM, 0.7 mM, 0.8 mM, 0.9 mM, or 1.0 mM.

In some embodiments, the pharmaceutical composition comprises an EZH2 inhibitor that is EPZ011989 and a GSK3 Inhibitor that is CHIR99021. The EPZ011989 is at a concentration of about 0.001 μM to 1,000 mM, about 0.01 μM to 100,000 μM, about 0.1 μM to 10,000 μM, about 1 μM to 1,000 μM, about 1 μM to 10 μM, about 10 μM to 100 μM, about 100 μM to 1 mM, or about 1 mM to 10 mM. and the CHIR99021 at a concentration of about 0.001 mM to 10,000 mM, about 0.01 mM to 1,000 mM, about 0.1 mM to 100 mM, about 0.001 mM to 0.01 mM, about 0.01 mM to 0.1 mM about 0.1 mM to 1 mM, about 1 mM to 10 mM, about 10 mM to 100 mM, about 100 mM to 1,000 mM, or about 1,000 mM to 10,000 mM.

In some embodiments, the EPZ011989 at a concentration of about 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, 10 mM, 12 mM, 14 mM, 16 mM, 18 mM, 20 mM, 25 mM, or about 30 mM and the CHIR99021 is at a concentration of about 1 mM, 2 mM, 3 mM, 4 mM, 5 mM, 6 mM, 7 mM, 8 mM, 9 mM, or 10 mM.

In some embodiments, the pharmaceutical composition comprises an EZH2 inhibitor that is UNC 2399 and a GSK3 Inhibitor that is AZD1080. The UNC 2399 is at a concentration of about 0.001 μM to 1,000 mM, about 0.01 μM to 100,000 μM, about 0.1 μM to 10,000 μM, about 1 μM to 1,000 μM, about 1 μM to 10 μM, about 10 μM to 100 μM, about 100 μM to 1 mM, or about 1 mM to 10 mM. and the AZ1090 is at a concentration of about 0.001 mM to 10,000 mM, about 0.01 mM to 1,000 mM, about 0.1 mM to 100 mM, about 0.001 mM to 0.01 mM, about 0.01 mM to 0.1 mM, about 0.1 mM to 1 mM, about 1 mM to 10 mM, about 10 mM to 100 mM, about 100 mM to 1,000 mM, or about 1,000 mM to 10,000 mM.

In some embodiments, the UNC 2399 at a concentration of about 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, 10 mM, 12 mM, 14 mM, 16 mM, 18 mM, 20 mM, 25 mM, or about 30 mM and the AZ1090 is at a a concentration of about 1 mM, 2 mM, 3 mM, 4 mM, 5 mM, 6 mM, 7 mM, 8 mM, 9 mM, or 10 mM.

In some embodiments, the pharmaceutical composition comprises an EZH2 inhibitor that is UNC 2399 and a GSK3 Inhibitor that is LY2090314. The UNC 2399 is at a concentration of about 0.001 μM to 1,000 mM, about 0.01 μM to 100,000 μM, about 0.1 μM to 10,000 μM, about 1 μM to 1,000 μM, about 1 μM to 10 μM about 10 μM to 100 μM, about 100 μM to 1 mM, or about 1 mM to 10 mM. and the LY2090314 at a concentration of about 0.001 μM to 10 mM, about 0.01 μM to 1 mM, about 0.1 μM to 100 uM, about 0.001 μM to 0.01 μM, about 0.01 μM to 0.1 μM, about 0.1 μM to 1 μM, about 1 μM to 10 μM, about 10 μM to 100 μM, about 100 μM to 1 mM, or about 1 mM to 10 mM.

In some embodiments, the UNC, 2399 at a concentration of about 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, 10 mM, 12 mM, 14 mM, 16 mM, 18 mM, 20 mM, 25 mM, or about 30 mM and the LY2090314 the is at a concentration of about 1 μM, 5 μM, 10 μM, 15 μM, 20 μM, or 40 μM.

In some embodiments, the pharmaceutical composition comprises an EZH2 inhibitor that is UNC 2399 and a GSK3 Inhibitor that is that is a substituted 3-Imidazo[1,2-a]pyridin-3-yl-4-(1,2,3,4-tetrahydro-[1,4]diazepino-[6,7,1-hi]indol-7-yl)pyrrole-2,5-dione. The UNC 2399 is at a concentration of about 0.001 μM to 1,000 mM, about 0.01 μM to 100,000 μM, about 0.1 μM to 10,000 μM, about 1 μM to 1,000 μM, about 1 μM to 10 μM, about 10 μM to 100 μM, about 100 μM to 1 mM, or about 1 mM to 10 mM. and the substituted 3-Imidazo[1,2-a]pyridin-3-yl-4-(1,2,3,4-tetrahydro-[1,4]diazepino-[6,7,1-hi]indol-7-yl)pyrrole-2,5-dione at a concentration of about 0.001 μM to 10 mM, about 0.01 μM to 1 mM, about 0.1 μM to 100 uM, about 0.001 μM to 0.01 μM, about 0.01 μM to 0.1 μM, about 0.1 μM to 1 μM, about 1 μM to 10 μM, about 10 μM to 100 μM, about 100 μM to 1 mM, or about 1 mM to 10 mM.

In some embodiments, the UNC 2399 at a concentration of about 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM or about 10 mM and the substituted 3-Imidazo[1,2-a]pyridin-3-yl-4-(1,2,3,4-tetrahydro-[1,4]diazepino-[6,7,1-hi]indol-7-yl)pyrrole-2,5-dione, is at a concentration of about 1 μM, 5 μM, 10 μM, 15 μM, 20 μM, 50 μM, 100 μM, 250 μM or 500 μM.

In some embodiments, the pharmaceutical composition comprises an EZH2 inhibitor that is UNC 2399 and a GSK3 Inhibitor that is GSK3-inhibitor XXII. The UNC 2399 is at a concentration of about 0.001μM to 1,000 mM, about 0.01 μM to 100,000 μM, about 0.1 μM to 10,000 μM, about 1 μM to 1,000 μM, about 1 μM to 10 μM, about 10 μM to 100 μM, about 100 μM to 1 mM, or about 1 mM to 10 mM. and the GSK3-inhibitor XXII, at a concentration of about of about 0.1 μM to 1,000 mM, about 1 μM to 100 mM, about 10 μM to 10 mM, about 0.1 μM to 1 μM, about 1 μM to 10 μM, about 10 μM to 100 μM, about 100 μM to 1 mM, about 1 mM to 10 mM, about 10 mM to 100 mM, or about 100 mM to 1000 mM.

In some embodiments, the UNC 2399 at a concentration of about 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, 10 mM, 12 mM, 14 mM, 16 mM, 18 mM, 20 mM, 25 mM, or about 30 mM and the GSK3-inhibitor XXII is at a concentration of about 0.1 mM, 0.2 mM, 0.3 mM, 0.4 mM, 0.5 mM, 0.6 mM, 0.7 mM, 0.8 mM, 0.9 mM, or 1.0 mM.

In some embodiments, the pharmaceutical composition comprises an EZH2 inhibitor that is UNC 2399 and a GSK3 Inhibitor that is CHIR99021. The UNC 2399 is at a concentration of about 0.001 μM to 1,000 mM, about 0.01 μM to 100,000 μM, about 0.1 μM to 10,000 μM, about 1 μM to 1,000 μM, about 1 μM to 10 μM, about 10 μM to 100 μM, about 100 μM to 1 mM, or about 1 mM to 10 mM. and the CHIR99021 at a concentration of about 0.001 mM to 10,000 mM, about 0.01 mM to 1,000 mM, about 0.1 mM to 100 mM, about 0.001 mM to 0.01 mM, about 0.01 mM to 0.1 mM, about 0.1 mM to 1 mM, about 1 mM to 10 mM, about 10 mM to 100 mM, about 100 mM to 1,000 mM, or about 1,000 mM to 10,000 mM.

In some embodiments, the UNC 2399 at a concentration of about 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, 10 mM, 12 mM, 14 mM, 16 mM, 18 mM, 20 mM, 25 mM, or about 30 mM and the CHIR99021 is at a concentration of about 1 mM, 2 mM, 3 mM, 4 mM, 5 mM, 6 mM, 7 mM, 8 mM, 9 mM, or 10 mM.

In some embodiments, the pharmaceutical composition comprises a EZH2 inhibitor that is PF-06726304 and a GSK3 Inhibitor that is AZD1080. The PF-06726304 is at a concentration of about 0.001 μM to 1,000 mM, about 0.01 μM to 100,000 μM, about 0.1 μM to 10,000 μM, about 1 μM to 1,000 μM, about 1 μM to 10 μM, about 10 μM to 100 μM, about 100 μM to 1 mM, or about 1 mM to 10 mM. and the AZ1090 is at a concentration of about 0.001 mM to 10,000 mM, about 0.01 mM to 1,000 mM, about 0.1 mM to 100 mM, about 0.001 mM to 0.01 mM, about 0.01 mM to 0.1 mM, about 0.1 mM to 1 mM, about 1 mM to 10 mM, about 10 mM to 100 mM, about 100 mM to 1,000 mM, or about 1,000 mM to 10,000 mM.

In some embodiments, the PF-06726304 at a concentration of about 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, 10 mM, 12 mM, 14 mM, 16 mM, 18 mM, 20 mM, 25 mM, or about 30 mM and the AZ1090 is at a a concentration of about 1 mM, 2 mM, 3 mM, 4 mM, 5 mM, 6 mM, 7 mM, 8 mM, 9 mM, or 10 mM.

In some embodiments, the pharmaceutical composition comprises an EZH2 inhibitor that is PF-06726304 and a GSK3 Inhibitor that is LY2090314. The PF-06726304 is at a concentration of about 0.001 μM to 1,000 mM, about 0.01 μM to 100,000 μM, about 0.1 μM to 10,000 μM, about 1 μM to 1,000 μM, about 1 μM to 10 μM, about 10 μM to 100 μM, about 100 μM to 1 mM, or about 1 mM to 10 mM. and the LY2090314 at a concentration of about 0.001 μM to 10 mM, about 0.01 μM to 1 mM, about 0.1 μM to 100 uM, about 0.001 μM to 0.01 μM, about 0.01 μM to 0.1 μM, about 0.1 μM to 1 μM, about 1 μM to 10 μM, about 10 μM to 100 μM, about 100 μM to 1 mM, or about 1 mM to 10 mM.

In some embodiments, the PF-06726304 at a concentration of about 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, 10 mM, 12 mM, 14 mM, 16 mM, 18 mM, 20 mM, 25 mM, or about 30 mM and the LY2090314 the is at a concentration of about 1 μM, 5 μM, 10 μM, 15 μM, 20 μM, or 40 μM.

In some embodiments, the pharmaceutical composition comprises a EZH2 inhibitor that is PF-06726304 and a GSK3 Inhibitor that is that is a substituted 3-Imidazo[1,2-a]pyridin-3-yl-4-(1,2,3,4-tetrahydro-[1,4]diazepino-[6,7,1-hi]indol-7-yl)pyrrole-2,5-dione. The PE-06726304 is at a concentration of about 0.001 μM to 1,000 mM, about 0.01 μM to 100,000 μM, about 0.1 μM to 10,000 μM, about 1 μM to 1,000 μM, about 1 μM to 10 μM, about 10 μM to 100 μM, about 100 μM to 1 mM, or about 1 mM to 10 mM. and the substituted 3-Imidazo[1,2-a]pyridin-3-yl-4-(1,2,3,4-tetrahydro-[1,4]diazepino-[6,7,1-hi]indol-7-yl)pyrrole-2,5-dione at a concentration of about 0.001 μM to 10 mM, about 0.01 μM to 1 mM, about 0.1 μM to 100 uM, about 0.001 μM to 0.01 μM, about 0.01 μM to 0.1 μM, about 0.1 μM to 1 μM, about 1 μM to 10 μM, about 10 μM to 100 μM, about 100 μM to 1 mM, or about 1 mM to 10 mM.

In some embodiments, the PF-06726304 at a concentration of about 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM or about 10 mM and the substituted 3-Imidazo[1,2-a]pyridin-3-yl-4-(1,2,3,4-tetrahydro-[1,4]diazepino-[6,7,1-hi]indol-7-yl)pyrrole-2,5-dione, is at a concentration of about 1 μM, 5 μM, 10 μM, 15 μM, 20 μM, 50 μM, 100 μM, 250 μM or 500 μM.

In some embodiments, the pharmaceutical composition comprises an EZH2 inhibitor that is PF-06726304 and a GSK3 Inhibitor that is GSK3-inhibitor XXII. The PE-06726304 is at a concentration of about 0.001 μM to 1,000 mM, about 0.01 μM to 100,000 μM, about 0.1 μM to 10,000 μM, about 1 μM to 1,000 μM, about 1 μM to 10 μM, about 10 μM to 100 μM, about 100 μM to 1 mM, or about 1 mM to 10 mM, and the GSK3-inhibitor XXII, at a concentration of about of about 0.1 μM to 1,000 mM, about 1 μM to 100 mM, about 10 μM to 10 mM, about 0.1 μM to 1 μM, about 1 μM to 1.0 μM, about 10 μM to 100 μM, about 100 μM to 1 mM, about 1 mM to 10 mM, about 10 mM to 100 mM, or about 100 mM to 1000 mM.

In some embodiments, the PF-06726304 at a concentration of about 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, 10 mM, 12 mM, 14 mM, 16 mM, 18 mM, 20 mM, 25 mM, or about 30 mM, and the GSK3-inhibitor XXII is at a concentration of about 0.1 mM, 0.2 mM, 0.3 mM, 0.4 mM, 0.5 mM, 0.6 mM, 0.7 mM, 0.8 mM, 0.9 mM, or 1.0 mM.

In some embodiments, the pharmaceutical composition comprises an EZH2 inhibitor that is PF-06726304 and a GSK3 Inhibitor that is CHIR99021. The PF-06726304 is at a concentration of about 0.001 μM to 1,000 mM, about 0.01 μM to 100,000 μM, about 0.1 μM to 10,000 μM, about 1 μM to 1,000 μM, about 1 μM to 10 μM, about 10 μM to 100 μM, about 100 μM to 1 mM, or about 1 mM to 10 mM. and the CHIR99021 at a concentration of about 0.001 mM to 10,000 mM, about 0.01 mM to 1,000 mM, about 0.1 mM to 100 mM, about 0.001 mM to 0.01 mM, about 0.01 mM to 0.1 mM, about 0.1 mM to 1 mM, about 1 mM to 10 mM, about 10 mM to 100 mM, about 100 mM to 1,000 mM, or about 1,000 mM to 10,000 mM.

In some embodiments, the PF-06726304 at a concentration of about 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, 10 mM, 12 mM, 14 mM, 16 mM, 18 mM, 20 mM, 25 mM, or about 30 mM and the CHIR99021 is at a concentration of about 1 mM, 2 mM, 3 mM, 4 mM, 5 mM, 6 mM, 7 mM, 8 mM, 9 mM, or 10 mM.

In some embodiments, the pharmaceutical composition comprises an EZH2 inhibitor that is CPI-1205 and a GSK3 Inhibitor that is AZD1080 and a HDAC inhibitor that is VPA. The CPI-1205 is at a concentration of about 0.001 μM to 1,000 mM, about 0.01 μM to 100,000 μM, about 0.1 μM to 10,000 μM, about 1 μM to 1,000 μM, about 1 μM to 10 μM, about 10 μM to 100 μM, about 100 μM to 1 mM, or about 1 mM to 10 mM. and the AZ1090 is at a concentration of about 0.001 mM to 10,000 mM, about 0.01 mM to 1,000 mM, about 0.1 mM to 100 mM, about 0.001 mM to 0.01 mM, about 0.01 mM to 0.1 mM, about 0.1 mM to 1 mM, about 1 mM to 10 mM, about 10 mM to 100 mM, about 100 mM to 1,000 mM, or about 1,000 mM to 10,000 mM and the VPA at a concentration about 100 mM to 4,000 mM.

In some embodiments, the CPI-1205 at a concentration of about 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, 10 mM, 12 mM, 14 mM, 16 mM, 18 mM, 20 mM, 25 mM, or about 30 mM and the AZ1090 is at a a concentration of about 1 mM, 2 mM, 3 mM, 4 mM, 5 mM, 6 mM, 7 mM, 8 mM, 9 mM, or 10 mM and the VPA at a concentration about 100 mM to 4,000 mM.

In some embodiments, the pharmaceutical composition comprises an EZH2 inhibitor that is CPI-1205 and a GSK3 Inhibitor that is LY2090314 and a HDAC inhibitor that is VPA. The CPI-1205 is at a concentration of about 0.001 μM to 1,000 mM, about 0.01 μM to 100,000 μM, about 0.1 μM to 10,000 μM, about 1 μM to 1,000 μM, about 1 μM to 10 μM, about 10 μM to 100 μM, about 100 μM to 1 mM, or about 1 mM to 10 mM. and the LY2090314 at a concentration of about 0.001 μM to 10 mM, about 0.01 μM to 1 mM, about 0.1 μM to 100 uM, about 0.001 μM to 0.01 μM, about 0.01 μM to 0.1 μM, about 0.1 μM to 1 μM, about 1 μM to 10 μM, about 10 μM to 100 μM, about 100 μM to 1 mM, or about 1 mM to 10 mM and the VPA at a concentration about 100 mM to 4,000 mM.

In some embodiments, the CPI-1205 at a concentration of about 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, 10 mM, 12 mM, 14 mM, 16 mM, 18 mM, 20 mM, 25 mM, or about 30 mM and the LY2090314 the is at a concentration of about 1 μM, 5 μM, 10 μM, 15 μM, 20 μM or 40 μM and the VPA at a concentration about 100 mM to 4,000 mM.

In some embodiments, the pharmaceutical composition comprises an EZH2 inhibitor that is CPI-1205 and a GSK3 Inhibitor that is that is a substituted 3-Imidazo[1,2-a]pyridin-3-yl-4-(1,2,3,4-tetrahydro-[1,4]diazepino-[6,7,1-hi]indol-7-yl)pyrrole-2,5-dione and a HDAC inhibitor that is VPA. The CPI-1205 is at a concentration of about 0.001 μM to 1,000 mM, about 0.01 μM to 100,000 μM, about 0.1 μM to 10,000 μM, about 1 μM to 1,000 μM, about 1 μM to 10 μM, about 10 μM to 100 μM, about 100 μM to 1 mM, or about 1 mM to 10 mM. and the substituted 3-Imidazo[1,2-a]pyridin-3-yl-4-(1,2,3,4-tetrahydro-[1,4]diazepino-[6,7,1-hi]indol-7-yl)pyrrole-2,5-dione at a concentration of about 0.001 μM to 10 mM, about 0.01 μM to 1 mM, about 0.1 μM to 100 uM, about 0.001 μM to 0.01 μM, about 0.01 μM to 0.1 μM, about 0.1 μM to 1 μM, about 1 μM to 10 μM, about 10 μM to 100 μM, about 100 μM to 1 mM, or about 1 mM to 10 mM and the VPA at a concentration about 100 mM to 4,000 mM.

In some embodiments, the CPI-1205 at a concentration of about 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, 10 mM, 12 mM, 14 mM, 16 mM, 18 mM, 20 mM, 25 mM, or about 30 mM and the substituted 3-Imidazo[1,2-a]pyridin-3-yl-4-(1,2,3,4-tetrahydro-[1,4]diazepino-[6,7,1-hi]indol-7-yl)pyrrole-2,5-dione, is at a concentration of about 1 μM, 5 μM, 10 μM, 15 μM, 20 μM, 50 μM, 100 μM, 250 μM or 500 μM and the VPA at a concentration about 100 mM to 4,000 mM.

In some embodiments, the pharmaceutical composition comprises an EZH2 inhibitor that is CPI-1205 and a GSK3 Inhibitor that is GSK3-inhibitor XXII and a HDAC inhibitor that is VPA. The CPI-1205 is at a concentration of about 0.001 μM to 1,000 mM, about 0.01 μM to 100,000 μM, about 0.1 μM to 10,000 μM, about 1 μM to 1,000 μM, about 1 μM to 10 μM, about 10 μM to 100 μM about 100 μM to 1 mM, or about 1 mM to 10 mM, and the GSK3-inhibitor XXII, at a concentration of about of about 0.1 μM to 1,000 mM, about 1 μM to 100 mM, about 10 μM to 10 mM, about 0.1 μM to 1 μM, about 1 μM to 10 μM, about 10 μM to 100 μM, about 100 μM to 1 mM, about 1 mM to 10 mM, about 10 mM to 100 mM, or about 100 mM to 1000 mM and the VPA at a concentration about 100 mM to 4,000 mM.

In some embodiments, the CPI-1205 at a concentration of about 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, 10 mM, 12 mM, mM, 16 mM, 18 mM, 20 mM, 25 mM, or about 30 mM and the GSK3-inhibitor XXII is at a concentration of about 0.1 mM, 0.2 mM, 0.3 mM, 0.4 mM, 0.5 mM, 0.6 mM, 0.7 mM, 0.8 mM, 0.9 mM, or 1.0 mM. and the VPA at a concentration about 100 mM to 4,000 mM.

In some embodiments, the pharmaceutical composition comprises an EZH2 inhibitor that is CPI-1205 and a GSK3 Inhibitor that is CHIR99021 and a HDAC inhibitor that is VPA. The CPI-1205 is at a concentration of about 0.001 μM to 1,000 mM, about 0.01 μM to 100,000 μM, about 0.1 μM to 10,000 μM, about 1 μM to 1,000 μM, about 1 μM to 10 μM, about 10 μM to 100 μM, about 100 μM to 1 mM, or about 1 mM to 10 mM. and the CHIR99021 at a concentration of about 0.001 mM to 10,000 mM, about 0.01 mM to 1,000 mM, about 0.1 mM to 100 mM, about 0.001 mM to 0.01 mM, about 0.01 mM to 0.1 mM, about 0.1 mM to 1 mM, about 1 mM to 10 mM, about 10 mM to 100 mM, about 100 mM to 1,000 mM, or about 1,000 mM to 10,000 mM and the VPA at a concentration about 100 mM to 4,000 mM.

In some embodiments, the CPI-1205 at a concentration of about 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, 10 mM, 12 mM, 14 mM, 16 mM, 18 mM, 20 mM, 25 mM, or about 30 mM and the CHIR99021 is at a concentration of about 1 mM, 2 mM, 3 mM, 4 mM, 5 mM, 6 mM, 7 mM, 8 mM, 9 mM, or 10 mM and the VPA at a concentration about 100 mM to 4,000 mM.

In some embodiments, the pharmaceutical composition comprises an EZH2 inhibitor that is CPI-169 and a GSK3 Inhibitor that is AZD1080 and a HDAC inhibitor that is VPA. The CPI-169 is at a concentration of about 0.001 μM to 1,000 mM, about 0.01 μM to 100,000 μM, about 0.1 μM to 10,000 μM, about 1 μM to 1,000 μM, about 1 μM to 10 μM, about 10 μM to 100 μM, about 100 μM to 1 mM, or about 1 mM to 10 mM. and the AZ1090 is at a concentration of about 0.001 mM to 10,000 mM, about 0.01 mM to 1,000 mM, about 0.1 mM to 100 mM, about 0.001 mM to 001 mM, about 0.01 mM to 0.1 mM, about 0.1 mM to 1 mM, about 1 mM to 10 mM, about 10 mM to 100 mM, about 100 mM to 1,000 mM, or about 1,000 mM to 10,000 mM and the VPA at a concentration about 100 mM to 4,000 mM.

In some embodiments, the CPI-169 at a concentration of about 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, 10 mM, 12 mM, 14 mM, 16 mM, 18 mM, 20 mM, 25 mM, or about 30 mM and the AZ1090 is at a a concentration of about 1 mM, 2 mM, 3 mM, 4 mM, 5 mM, 6 mM, 7 mM, 8 mM, 9 mM, or 10 mM and the VPA at a concentration about 100 mM to 4,000 mM.

In some embodiments, the pharmaceutical composition comprises an EZH2 inhibitor that is CPI-169 and a GSK3 Inhibitor that is LY2090314 and a HDAC inhibitor that is VPA. The CPI-169 is at a concentration of about 0.001 μM to 1,000 mM, about 0.01 μM to 100,000 μM, about 0.1 μM to 10,000 μM, about 1 μM to 1,000 μM, about 1 μM to 10 μM, about 10 μM to 100 μM, about 100 μM to 1 mM, or about 1 mM to 10 mM. and the LY2090314 at a concentration of about 0.001 μM to 10 mM, about 0.01 μM to 1 mM, about 0.1 μM to 100 uM, about 0.001 μM to 0.01 μM, about 0.01 μM to 0.1 μM, about 0.1 μM to 1 μM, about 1 μM to 10 μM, about 10 μM to 100 μM, about 100 μM to 1 mM, or about 1 mM to 10 mM and the VPA at a concentration about 100 mM to 4,000 mM.

In some embodiments, the CPI-169 at a concentration of about 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, 10 mM, 12 mM, 14 mM, 16 mM, 18 mM, 20 mM, 25 mM, or about 30 mM and the LY2090314 the is at a concentration of about 1 μM, 5 μM, 10 μM, 15 μM, 20 μM or 40 μM and the VPA at a concentration about 100 mM to 4,000 mM.

In some embodiments, the pharmaceutical composition comprises an EZH2 inhibitor that is CPI-169 and a GSK3 Inhibitor that is that is a substituted 3-Imidazo[1,2-a]pyridin-3-yl-4-(1,2,3,4-tetrahydro-[1,4]diazepino-[6,7,1-hi]indol-7-yl)pyrrole-2,5-dione and a HDAC inhibitor that is VPA. The CPI-169 is at a concentration of about 0.001 μM to 1,000 mM, about 0.01 μM to 100,000 μM, about 0.1 μM to 10,000 μM, about 1 μM to 1,000 μM, about 1 μM to 10 μM, about 10 μM to 100 μM, about 100 μM to 1 mM, or about 1 mM to 10 mM. and the substituted 3-Imidazo[1,2-a]pyridin-3-yl-4-(1,2,3,4-tetrahydro-[1,4]diazepino-[6,7,1-hi]indol-7-yl)pyrrole-2,5-dione at a concentration of about 0.001 μM to 10 mM, about 0.01 μM to 1 mM, about 0.1 μM to 100 uM, about 0.001 μM to 0.01 μM, about 0.01 μM to 0.1 μM, about 0.1 μM to 1 μM, about 1 μM to 10 μM, about 10 μM to 100 μM, about 100 μM to 1 mM, or about 1 mM to 10 mM and the VPA at a concentration about 100 mM to 4,000 mM.

In some embodiments, the CPI-169 at a concentration of about 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, 10 mM, 12 mM, 14 mM, 16 mM, 18 mM, 20 mM, 25 mM, or about 30 mM and the substituted 3-Imidazo[1,2-a]pyridin-3-yl-4-(1,2,3,4-tetrahydro-[1,4]diazepino-[6,7,1-hi]indol-7-yl)pyrrole-2,5-dione, is at a concentration of about 1 μM, 5 μM, 10 μM, 15 μM, 20 μM, 50 μM, 100 μM, 250 μM or 500 μM and the VPA at a concentration about 100 mM to 4,000 mM.

In some embodiments, the pharmaceutical composition comprises an EZH2 inhibitor that is CPI-169 and a GSK3 Inhibitor that is GSK3-inhibitor XXII and a HDAC inhibitor that is VPA. The CPI-169 is at a concentration of about 0.001 μM to 1,000 mM, about 0.01 μM to 100,000 μM, about 0.1 μM to 10,000 μM, about 1 μM to 1,000 μM, about 1 μM to 10 μM, about 10 μM to 100 μM, about 100 μM to 1 mM, or about 1 mM to 10 mM. and the GSK3-inhibitor XXII, at a concentration of about of about 0.1 μM to 1,000 mM, about 1 μM to 100 mM, about 10 μM to 10 mM, about 0.1 μM to 1 μM, about 1 μM to 10 μM, about 10 μM to 100 μM, about 100 μM to 1 mM, about 1 mM to 10 mM, about 10 mM to 100 mM, or about 100 mM to 1000 mM and the VPA at a concentration about 100 mM to 4,000 mM.

In some embodiments, the CPI-169 at a concentration of about 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, 10 mM, 12 mM, 14 mM, 16 mM, 18 mM, 20 mM, 25 mM, or about 30 mM and the GSK3-inhibitor XXII is at a concentration of about 0.1 mM, 0.2 mM, 0.3 mM, 0.4 mM, 0.5 mM, 0.6 mM, 0.7 mM, 0.8 mM, 0.9 mM, or 1.0 mM. and the VPA at a concentration about 100 mM to 4,000 mM.

In some embodiments, the pharmaceutical composition comprises an EZH2 inhibitor that is CPI-169 and a GSK3 Inhibitor that is CHIR99021 and a HDAC inhibitor that is VPA. The CPI-169 is at a concentration of about 0.001 μM to 1,000 mM, about 0.01 μM to 100,000 μM, about 0.1 μM to 10,000 μM, about 1 μM to 1,000 μM, about 1 μM to 10 μM, about 10 μM to 100 μM, about 100 μM to 1 mM, or about 1 mM to 10 mM. and the CHIR99021 at a concentration of about 0.001 mM to 10,000 mM, about 0.01 mM to 1,000 mM, about 0.1 mM to 100 mM, about 0.001 mM to 0.01 mM, about 0.01 mM to 0.1 mM, about 0.1 mM to 1 mM, about 1 mM to 10 mM, about 10 mM to 100 mM, about 100 mM to 1,000 mM, or about 1,000 mM to 10,000 mM and the VPA at a concentration about 100 mM to 4,000 mM.

In some embodiments, the CPI-169 at a concentration of about 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, 10 mM, 12 mM, 14 mM, 16 mM, 18 mM, 20 mM, 25 mM, or about 30 mM and the CHIR99021 is at a concentration of about 1 mM, 2 mM, 3 mM, 4 mM, 5 mM, 6 mM, 7 mM, 8 mM, 9 mM, or 10 mM and the VPA at a concentration about 100 mM to 4,000 mM.

In some embodiments, the pharmaceutical composition comprises an EZH2 inhibitor that is EL1 and a GSK3 Inhibitor that is AZD1080 and a HDAC inhibitor that is VPA. The EL1 is at a concentration of about 0.001 μM to 1,000 mM, about 0.01 μM to 100,000 μM, about 0.1 μM to 10,000 μM, about 1 μM to 1,000 μM, about 1 μM to 10 μM, about 10 μM to 100 μM, about 100 μM to 1 mM, or about 1 mM to 10 mM. and the AZ1090 is at a concentration of about 0.001 mM to 10,000 mM, about 0.01 mM to 1,000 mM, about 0.1 mM to 100 mM, about 0.001 mM to 0.01 mM, about 0.01 mM to 0.1 mM, about 0.1 mM to 1 mM, about 1 mM to 10 mM, about 10 mM to 100 mM, about 100 mM to 1,000 mM, or about 1,000 mM to 10,000 mM and the VPA at a concentration about 100 mM to 4,000 mM.

In some embodiments, the EL1 at a concentration of about 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, 10 mM, 12 mM, 14 mM, 16 mM, 18 mM, 20 mM, 25 mM, or about 30 mM and the AZ1090 is at a a concentration of about 1 mM, 2 mM, 3 mM, 4 mM, 5 mM, 6 mM, 7 mM, 8 mM, 9 mM, or 10 mM and the VPA at a concentration about 100 mM to 4,000 mM.

In some embodiments, the pharmaceutical composition comprises an EZH2 inhibitor that is EL1 and a GSK3 Inhibitor that is LY2090314 and a HDAC inhibitor that is VPA. The EL1 is at a concentration of about 0.001 μM to 1,000 mM, about 0.01 μM to 100,000 μM, about 0.1 μM to 10,000 μM, about 1 μM to 1,000 μM, about 1 μM to 10 μM, about 10 μM to 100 μM, about 100 μM to 1 mM, or about 1 mM to 10 mM. and the LY2090314 at a concentration of about 0.001 μM to 10 mM, about 0.01 μM to 1 mM, about 0.1 μM to 100 uM, about 0.001 μM to 001 μM, about 0.01 μM to 0.1 μM, about 0.1 μM to 1 μM about 1 μM to 10 μM, about 10 μM to 100 μM, about 100 μM to 1 mM, or about 1 mM to 10 mM and the VPA at a concentration about 100 mM to 4,000 mM.

In some embodiments, the EL1 at a concentration of about 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, 10 mM, 12 mM, 14 mM, 16 mM, 18 mM 20 mM, 25 mM, or about 30 mM and the LY2090314 the is at a concentration of about 1 μM, 5 μM, 10 μM, 15 μM, 20 μM or 40 μM and the VPA at a concentration about 100 mM to 4,000 mM.

In some embodiments, the pharmaceutical composition comprises an EZH2 inhibitor that is EL1 and a GSK3 Inhibitor that is that is a substituted 3-Imidazo[1,2-a]pyridin-3-yl-4-(1,2,3,4-tetrahydro-[1,4]diazepino-[6,7,1-hi]indol-7-yl)pyrrole-2,5-dione and a HDAC inhibitor that is VPA. The EL1 is at a concentration of about 0.001 μM to 1,000 mM, about 0.01 μM to 100,000 μM, about 0.1 μM to 10,000 μM, about 1 μM to 1,000 μM, about 1 μM to 10 μM, about 10 μM to 100 μM, about 100 μM to 1 mM, or about 1 mM to 10 mM. and the substituted 3-Imidazo[1,2-a]pyridin-3-yl-4-(1,2,3,4-tetrahydro-[1,4]diazepino-[6,7,1-hi]indol-7-yl)pyrrole-2,5-dione at a concentration of about 0.001 μM to 10 mM, about 0.01 μM to 1 mM, about 0.1 μM to 100 uM, about 0.001 μM to 0.01 μM, about 0.01 μM to 0.1 μM, about 0.1 μM to 1 μM, about 1 μM to 10 μM, about 10 μM to 100 μM, about 100 μM to 1 mM, or about 1 mM to 10 mM and the VPA at a concentration about 100 mM to 4,000 mM.

In some embodiments, the EL1 at a concentration of about 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, 10 mM, 12 mM, 14 mM, 16 mM, 18 mM, 20 mM, 25 mM, or about 30 mM and the substituted 3-Imidazo[1,2-a]pyridin-3-yl-4-(1,2,3,4-tetrahydro-[1,4]diazepino-[6,7,1-hi]indol-7-yl)pyrrole-2,5-dione, is at a concentration of about 1 μM, 10 μM, 15 μM, 20 μM, 50 μM, 100 μM, 250 μM or 500 μM and the VPA at a concentration about 100 mM to 4,000 mM.

In some embodiments, the pharmaceutical composition comprises an EZH2 inhibitor that is EL1 and a GSK3 Inhibitor that is GSK3-inhibitor XXII and a IIDAC inhibitor that is VPA. The EL1 is at a concentration of about 0.001 μM to 1,000 mM, about 0.01 μM to 100,000 μM, about 0.1 μM to 10,000 μM, about 1 μM to 1,000 μM, about 1 μM to 10 μM, about 10 μM to 100 μM, about 100 μM to 1 mM, or about 1 mM to 10 mM. and the GSK3-inhibitor XXII, at a concentration of about of about 0.1 μM to 1,000 mM, about 1 μM to 100 mM, about 10 μM to 10 mM, about 0.1 μM to 1 μM, about 1 μM to 10 μM, about 10 μM to 100 uM, about 100 μM to 1 mM, about 1 mM to 10 mM, about 10 mM to 100 mM, or about 100 mM to 1000 mM and the VPA at a concentration about 100 mM to 4,000 mM.

In some embodiments, the EL1 at a concentration of about 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, 10 mM, 12 mM, 14 mM, 16 mM, 18 mM, 20 mM, 25 mM, or about 30 mM and the GSK3-inhibitor XXII is at a concentration of about 0.1 mM, 0.2 mM, 0.3 mM, 0.4 mM, 0.5 mM, 0.6 mM, 0.7 mM, 0.8 mM, 0.9 mM, or 1.0 mM, and the VPA at a concentration about 100 mM to 4,000 mM.

In some embodiments, the pharmaceutical composition comprises an EZH2 inhibitor that is EL1 and a GSK3 Inhibitor that is CHIR99021 and a HDAC inhibitor that is VPA. The EL1 is at a concentration of about 0.001 μM to 1,000 mM, about 0.01 μM to 100,000 μM, about 0.1 μM to 10,000 μM, about 1 μM to 1,000 μM, about 1 μM to 10 μM, about 10 μM to 100 μM, about 100 μM to 1 mM, or about 1 mM to 10 mM. and the CHIR99021 at a concentration of about 0.001 mM to 10,000 mM, about 0.01 mM to 1,000 mM, about 0.1 mM to 100 mM, about 0.001 mM to 0.01 mM, about 0.01 mM to 0.1 mM, about 0.1 mM to 1 mM, about 1 mM to 10 mM, about 10 mM to 100 mM, about 100 mM to 1,000 mM, or about 1,000 mM to 10,000 mM and the VPA at a concentration about 100 mM to 4,000 mM.

In some embodiments, the EL1 at a concentration of about 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, 10 mM, 12 mM, 14 mM, 16 mM, 18 mM, 20 mM, 25 mM, or about 30 mM and the CHIR99021 is at a concentration of about 1 mM, 2 mM, 3 mM, 4 mM, 5 mM, 6 mM, 7 mM, 8 mM, 9 mM, or 10 mM and the VPA at a concentration about 100 mM to 4,000 mM.

In some embodiments, the pharmaceutical composition comprises an EZH2 inhibitor that is tazemetostat and a GSK3 Inhibitor that is AZD1080 and a HDAC inhibitor that is VPA. The tazemetostat is at a concentration of about 0.001 μM to 1,000 mM, about 0.01 μM to 100,000 μM, about 0.1 μM to 10,000 μM, about 1 μM to 1,000 μM, about 1 μM to 10 μM, about 10 μM to 100 μM, about 100 μM to 1 mM, or about 1 mM to 10 mM. and the AZ1090 is at a concentration of about 0.001 mM to 10,000 mM, about 0.01 mM to 1,000 mM, about 0.1 mM to 100 mM, about 0.001 mM to 0.01 mM, about 0.01 mM to 0.1 mM, about 0.1 mM to 1 mM, about 1 mM to 10 mM, about 10 mM to 100 mM, about 100 mM to 1,000 mM, or about 1,000 mM to 10,000 mM and the VPA at a concentration about 100 mM to 4,000 mM.

In some embodiments, the tazemetostat at a concentration of about 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, 10 mM, 12 mM, 14 mM, 16 mM, 18 mM, 20 mM, 25 mM, or about 30 mM and the AZ1090 is at a a concentration of about 1 mM, 2 mM, 3 mM, 4 mM, 5 mM, 6 mM, 7 mM, 8 mM, 9 mM, or 10 mM and the VPA at a concentration about 100 mM to 4,000 mM.

In some embodiments, the pharmaceutical composition comprises an EZH2 inhibitor that is tazemetostat and a GSK3 Inhibitor that is LY2090314 and a HDAC inhibitor that is VPA. The tazemetostat is at a concentration of about 0.001 μM to 1,000 mM, about 0.01 μM to 100,000 μM, about 0.1 μM to 10,000 μM, about 1 μM to 1,000 μM, about 1 μM to 10 μM, about 10 μM to 100 μM, about 100 μM to 1 mM, or about 1 mM to 10 mM. and the LY2090314 at a concentration of about 0.001 μM to 10 mM, about 0.01 μM to 1 mM, about 0.1 μM to 100 uM, about 0.001 μM to 0.01 μM, about 0.01 μM to 0.1 μM, about 0.1 μM to 1 μM, about 1 μM to 10 μM, about 10 μM to 100 μM about 100 μM to 1 mM, or about 1 mM to 10 mM and the VPA at a concentration about 100 mM to 4,000 mM.

In some embodiments, the tazemetostat at a concentration of about 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, 10 mM, 12 mM, 14 mM, 16 mM, 18 mM, 20 mM, 25 mM, or about 30 mM and the LY2090314 the is at a concentration of about 1 μM, 5 μM, 10 μM, 15 μM, 20 μM or 40 μM and the VPA at a concentration about 100 mM to 4,000 mM.

In some embodiments, the pharmaceutical composition comprises an EZH2 inhibitor that is tazemetostat and a GSK3 Inhibitor that is that is a substituted 3-Imidazo[1,2-a]pyridin-3-yl-4-(1,2,3,4-tetrahydro-[1,4]diazepino-[6,7,1-hi]indol-7-yl)pyrrole-2,5-dione and a HDAC inhibitor that is VPA. The tazemetostat is at a concentration of about 0.001 μM to 1,000 mM, about 0.01 μM to 100,000 μM, about 0.1 μM to 10,000 μM, about 1 μM to 1,000 μM, about 1 μM to 10 μM, about 10 μM to 100 μM, about 100 μM to 1 mM, or about 1 mM to 10 mM. and the substituted 3-Imidazo[1,2-a]pyridin-3-yl-4-(1,2,3,4-tetrahydro-[1,4]diazepino-[6,7,1-hi]indol-7-yl)pyrrole-2,5-dione at a concentration of about 0.001 μM to 10 mM, about 0.01 μM to 1 mM, about 0.1 μM to 100 uM, about 0.001 μM to 0.01 μM, about 0.01 μM to 0.1 μM, about 0.1 μM to 1 μM, about 1 μM to 10 μM, about 10 μM to 100 μM, about 100 μM to 1 mM, or about 1 mM to 10 mM. and the VPA at a concentration about 100 mM to 4,000 mM.

In some embodiments, the tazemetostat at a concentration of about 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, 10 mM, 12 mM, 14 mM, 16 mM, 18 mM, 20 mM, 25 mM, or about 30 mM and the substituted 3-Imidazo[1,2-a]pyridin-3-yl-4-(1,2,3,4-tetrahydro-[1,4]diazepino-[6,7,1-hi]indol-7-yl)pyrrole-2,5-dione, is at a concentration of about 1 μM, 5 μM, 10 μM, 15 μM, 20 μM, 50 μM, 100 μM, 250 μM or 500 μM and the VPA at a concentration about 100 mM to 4,000 mM.

In some embodiments, the pharmaceutical composition comprises an EZH2 inhibitor that is tazemetostat and a GSK3 Inhibitor that is GSK3-inhibitor XXII and a HDAC inhibitor that is VPA. The tazemetostat is at a concentration of about 0.001 μM to 1,000 mM, about 0.01 μM to 100,000 μM, about 0.1 μM to 10,000 μM, about 1 μM to 1,000 μM, about 1 μM to 10 μM, about 10 μM to 100 μM, about 100 μM to 1 mM, or about 1 mM to 10 mM. and the GSK3-inhibitor XXII, at a concentration of about of about 0.1 μM to 1,000 mM, about 1 μM to 100 mM, about 10 μM to 10 mM, about 0.1 μM to 1 μM, about 1 μM to 10 μM, about 10 μM to 100 μM, about 100 μM to 1 mM, about 1 mM to 10 mM, about 10 mM to 100 mM, or about 100 mM to 1000 mM and the VPA at a concentration about 100 mM to 4,000 mM.

In some embodiments, the tazemetostat at a concentration of about 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, 10 mM, 12 mM, 14 mM, 16 mM, 18 mM, 20 mM, 25 mM, or about 30 mM and the GSK3-inhibitor XXII is at a concentration of about 0.1 mM, 0.2 mM, 0.3 mM, 0.4 mM, 0.5 mM, 0.6 mM, 0.7 mM, 0.8 mM, 0.9 mM, or 1.0 mM. and the VPA at a concentration about 100 mM to 4,000 mM.

In some embodiments, the pharmaceutical composition comprises an EZH2 inhibitor that is tazemetostat and a GSK3 Inhibitor that is CHIR99021 and a HDAC inhibitor that is VPA. The tazemetostat is at a concentration of about 0.001 μM to 1,000 mM, about 0.01 μM to 100,000 μM, about 0.1 μM to 10,000 μM, about 1 μM to 1,000 μM, about 1 μM to 10 μM, about 10 μM to 100 μM, about 100 μM to 1 mM, or about 1 mM to 10 mM. and the CHIR99021 at a concentration of about 0.001 mM to 10,000 mM, about 0.01 mM to 1,000 mM, about 0.1 mM to 100 mM, about 0.001 mM to 0.01 mM, about 0.01 mM to 0.1 mM, about 0.1 mM to 1 mM, about 1 mM to 10 mM, about 10 mM to 100 mM, about 100 mM to 1,000 mM, or about 1,000 mM to 10,000 mM and the VPA at a concentration about 100 mM to 4,000 mM.

In some embodiments, the tazemetostat at a concentration of about 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, 10 mM, 12 mM, 14 mM, 16 mM, 18 mM, 20 mM, 25 mM, or about 30 mM and the CHIR99021 is at a concentration of about 1 mM, 2 mM, 3 mM, 4 mM, 5 mM, 6 mM, 7 mM, 8 mM, 9 mM, or 10 mM and the VPA at a concentration about 100 mM to 4,000 mM.

In some embodiments, the pharmaceutical composition comprises an EZH2 inhibitor that is valemetostat and a GSK3 Inhibitor that is AZD1080 and a HDAC inhibitor that is VPA. The valemetostat is at a concentration of about 0.001 μM to 1,000 mM, about 0.01 μM to 100,000 μM, about 0.1 μM to 10,000 μM, about 1 μM to 1,000 μM, about 1 μM to 10 μM, about 10 μM to 100 μM, about 100 μM to 1 mM, or about 1 mM to 10 mM. and the AZ1090 is at a concentration of about 0.001 mM to 10,000 mM, about 0.01 mM to 1,000 mM, about 0.1 mM to 100 mM, about 0.001 mM to 0.01 mM, about 0.01 mM to 0.1 mM, about 0.1 mM to 1 mM, about 1 mM to 10 mM, about 10 mM to 100 mM, about 100 mM to 1,000 mM, or about 1,000 mM to 10,000 mM and the VPA at a concentration about 100 mM to 4,000 mM.

In some embodiments, the valemetostat at a concentration of about 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, 10 mM, 12 mM, 14 mM, 16 mM, 18 mM, 20 mM, 25 mM, or about 30 mM and the AZ1090 is at a a concentration of about 1 mM, 2 mM, 3 mM, 4 mM, 5 mM, 6 mM, 7 mM, 8 mM, 9 mM, or 10 mM and the VPA at a concentration about 100 mM to 4,000 mM.

In some embodiments, the pharmaceutical composition comprises an EZH2 inhibitor that is valemetostat and a GSK3 Inhibitor that is LY2090314 and a HDAC inhibitor that is VPA. The valemetostat is at a concentration of about 0.001 μM to 1,000 mM, about 0.01 μM to 100,000 μM, about 0.1 μM to 10,000 μM, about 1 μM to 1,000 μM, about 1 μM to 10 μM, about 10 μM to 100 μM, about 100 μM to 1 mM, or about 1 mM to 10 mM. and the LY2090314 at a concentration of about 0.001 μM to 10 mM, about 0.01 μM to 1 mM, about 0.1 μM to 100 uM, about 0.001 μM to 0.01 μM, about 0.01 μM to 0.1 μM, about 0.1 μM to 1 μM, about 1 μM to 10 μM, about 10 μM to 100 μM, about 100 μM to 1 mM, or about 1 mM to 10 mM and the VPA at a concentration about 100 mM to 4,000 mM.

In some embodiments, the valemetostat at a concentration of about 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, 10 mM, 12 mM, 14 mM, 16 mM, 18 mM, 20 mM, 25 mM, or about 30 mM and the LY2090314 the is at a concentration of about 1 μM, 5 μM, 10 μM, 15 μM, 20 μM or 40 μM and the VPA at a concentration about 100 mM to 4,000 mM.

In some embodiments, the pharmaceutical composition comprises an EZH2 inhibitor that is valemetostat and a GSK3 Inhibitor that is that is a substituted 3-Imidazo[1,2-a]pyridin-3-yl-4-(1,2,3,4-tetrahydro-[1,4]diazepino-[6,7,1-hi]indol-7-yl)pyrrole-2,5-dione and a HDAC inhibitor that is VPA. The valemetostat is at a concentration of about 0.001 μM to 1,000 mM, about 0.01 μM to 100,000 μM, about 0.1 μM to 10,000 μM, about 1 μM to 1,000 μM, about 1 μM to 10 μM, about 10 μM to 100 μM, about 100 μM to 1 mM, or about 1 mM to 10 mM. and the substituted 3-Imidazo[1,2-a]pyridin-3-yl-4-(1,2,3,4-tetrahydro-[1,4]diazepino-[6,7,1-hi]indol-7-yl)pyrrole-2,5-dione at a concentration of about 0.001 μM to 10 mM, about 0.01 μM to 1 mM, about 0.1 μM to 100 uM, about 0.001 μM to 0.01 μM, about 0.01 μM to 0.1 μM, about 0.1 μM to 1 μM, about 1 μM to 10 μM, about 10 μM to 100 μM, about 100 μM to 1 mM, or about 1 mM to 10 mM and the VPA at a concentration about 100 mM to 4,000 mM.

In some embodiments, the valemetostat at a concentration of about 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, 10 mM, 12 mM, 14 mM, 16 mM, 18 mM, 20 mM, 25 mM, or about 30 mM and the substituted 3-Imidazo[1,2-a]pyridin-3-yl-4-(1,2,3,4-tetrahydro-[1,4]diazepino-[6,7,1-hi]indol-7-yl)pyrrole-2,5-dione, is at a concentration of about 1 μM, 5 μM, 10 μM, 15 μM, 20 μM, 50 μM, 100 μM, 250 μM or 500 μM and the VPA at a concentration about 100 mM to 4,000 mM.

In some embodiments, the pharmaceutical composition comprises an EZH2 inhibitor that is valemetostat and a GSK3 Inhibitor that is GSK3-inhibitor XXII and a HDAC inhibitor that is VPA. The valemetostat is at a concentration of about 0.001 μM to 1,000 mM, about 0.01 μM to 100,000 μM, about 0.1 μM to 10,000 μM, about 1 μM to 1,000 μM, about 1 μM to 10 μM, about 10 μM to 100 μM, about 100 μM to 1 mM, or about 1 mM to 10 mM. and the GSK3-inhibitor XXII, at a concentration of about of about 0.1 μM to 1,000 mM, about 1 μM to 100 mM, about 10 μM to 10 mM, about 0.1 μM to 1 μM, about 1 μM to 10 μM, about 10 μM to 100 μM, about 100 μM to 1 mM, about 1 mM to 10 mM, about 10 mM to 100 mM, or about 100 mM to 1000 mM and the VPA at a concentration about 100 mM to 4,000 mM.

In some embodiments, the valemetostat at a concentration of about 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, 10 mM, 12 mM, 14 mM, 16 mM, 18 mM, 20 mM, 25 mM, or about 30 mM and the GSK3-inhibitor XXII is at a concentration of about 0.1 mM, 0.2 mM, 0.3 mM, 0.4 mM, 0.5 mM, 0.6 mM, 0.7 mM, 0.8 mM, 0.9 mM, or 1.0 mM. and the VPA at a concentration about 100 mM to 4,000 mM.

In some embodiments, the pharmaceutical composition comprises an EZH2 inhibitor that is valemetostat and a GSK3 Inhibitor that is CHIR99021 and a HDAC inhibitor that is VPA. The valemetostat is at a concentration of about 0.001 μM to 1,000 mM, about 0.01 μM to 100,000 μM, about 0.1 μM to 10,000 μM, about 1 μM to 1,000 μM, about 1 μM to 10 μM, about 10 μM to 100 μM, about 100 μM to 1 mM, or about 1 mM to 10 mM. and the CHIR99021 at a concentration of about 0.001 mM to 10,000 mM, about 0.01 mM to 1,000 mM, about 0.1 mM to 100 mM, about 0.001 mM to 0.01 mM, about 0.01 mM to 0.1 mM, about 0.1 mM to 1 mM, about 1 mM to 10 mM, about 10 mM to 100 mM, about 100 mM to 1,000 mM, or about 1,000 mM to 10,000 mM and the VPA at a concentration about 100 mM to 4,000 mM.

In some embodiments, the valemetostat at a concentration of about 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, 10 mM, 12 mM, 14 mM, 16 mM, 18 mM, 20 mM, 25 mM, or about 30 mM and the CHIR99021 is at a concentration of about 1 mM, 2 mM, 3 mM, 4 mM, 5 mM, 6 mM, 7 mM, 8 mM, 9 mM, or 10 mM and the VPA at a concentration about 100 mM to 4,000 mM.

In some embodiments, the pharmaceutical composition comprises an EZH2 inhibitor that is PF-06821497 and a GSK3 Inhibitor that is AZD1080 and a HDAC inhibitor that is VPA. The PF-06821497 is at a concentration of about 0.001 μM to 1,000 mM, about 0.01 μM to 100,000 μM, about 0.1 μM to 10,000 μM, about 1 μM to 1,000 μM, about 1 μM to 10 μM, about 10 μM to 100 μM, about 100 μM to 1 mM, or about 1 mM to 10 mM. and the AZ1090 is at a concentration of about 0.001 mM to 10,000 mM, about 0.01 mM to 1,000 mM, about 0.1 mM to 100 mM, about 0.001 mM to 0.01 mM, about 0.01 mM to 0.1 mM, about 0.1 mM to 1 mM, about 1 mM to 10 mM, about 10 mM to 100 mM, about 100 mM to 1,000 mM, or about 1,000 mM to 10,000 mM and the VPA at a concentration about 100 mM to 4,000 mM.

In some embodiments, the PF-06821497 at a concentration of about 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, 10 mM, 12 mM, 14 mM, 16 mM, 18 mM, 20 mM, 25 mM, or about 30 mM and the AZ1090 is at a a concentration of about 1 mM, 2 mM, 3 mM, 4 mM, 5 mM, 6 mM, 7 mM, 8 mM, 9 mM, or 10 mM and the VPA at a concentration about 100 mM to 4,000 mM.

In some embodiments, the pharmaceutical composition comprises an EZH2 inhibitor that is PF-06821497 and a GSK3 Inhibitor that is LY2090314 and a HDAC inhibitor that is VPA. The PF-06821497 is at a concentration of about 0.001 μM to 1,000 mM, about 0.01 μM to 100,000 μM, about 0.1 μM to 10,000 μM, about 1 μM to 1,000 μM, about 1 μM to 10 μM, about 10 μM to 100 μM, about 100 μM to 1 mM, or about 1 mM to 10 mM. and the LY2090314 at a concentration of about 0.001 μM to 10 mM, about 0.01 μM to 1 mM, about 0.1 μM to 100 uM, about 0.001 μM to 0.01 μM, about 0.01 μM to 0.1 μM, about 0.1 μM to 1 μM, about 1 μM to 10 μM, about 10 μM to 100 μM, about 100 μM to 1 mM, or about 1 mM to 10 mM and the VPA at a concentration about 100 mM to 4,000 mM.

In some embodiments, the PF-06821497 at a concentration of about 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, 10 mM, 12 mM, 14 mM, 16 mM, 18 mM, 20 mM, 25 mM, or about 30 mM and the LY2090314 the is at a concentration of about 1 μM, 5 μM, 10 μM, 15 μM, 20 μM or 40 μM and the VPA at a concentration about 100 mM to 4,000 mM.

In some embodiments, the pharmaceutical composition comprises an EZH2 inhibitor that is PF-06821497 and a GSK3 Inhibitor that is that is a substituted 3-Imidazo[1,2-a]pyridin-3-yl-4-(1,2,3,4-tetrahydro-[1,4]diazepino-[6,7,1-hi]indol-7-yl)pyrrole-2,5-dione and a HDAC inhibitor that is VPA. The PF-06821497 is at a concentration of about 0.001 μM to 1,000 mM, about 0.01 μM to 100,000 μM, about 0.1 μM to 10,000 μM, about 1 μM to 1,000 μM, about 1 μM to 10 μM, about 10 μM to 100 μM, about 100 μM to 1 mM, or about 1 mM to 10 mM. and the substituted 3-Imidazo[1,2-a]pyridin-3-yl-4-(1,2,3,4-tetrahydro-[1,4]diazepino-[6,7,1-hi]indol-7-yl)pyrrole-2,5-dione at a concentration of about 0.001 μM to 10 mM, about 0.01 μM to 1 mM, about 0.1 μM to 100 uM, about 0.001 μM to 0.01 μM, about 0.01 μM to 0.1 μM, about 0.1 μM to 1 μM, about 1 μM to 10 μM, about 10 μM to 100 μM, about 100 μM to 1 mM, or about 1 mM to 10 mM. and the VPA at a concentration about 100 mM to 4,000 mM.

In some embodiments, the PF-06821497 at a concentration of about 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, 10 mM, 12 mM, 14 mM, 16 mM, 18 mM, 20 mM, 25 mM, or about 30 mM and the substituted 3-Imidazo[1,2-a]pyridin-3-yl-4-(1,2,3,4-tetrahydro-[1,4]diazepino-[6,7,1-hi]indol-7-yl)pyrrole-2,5-dione, is at a concentration of about 1 μM, 5 μM, 10 μM, 15 μM, 20 μM, 50 μM, 100 μM, 250 μM or 500 μM and the VPA at a concentration about 100 mM to 4,000 mM.

In some embodiments, the pharmaceutical composition comprises an EZH2 inhibitor that is PF-06821497 and a GSK3 Inhibitor that is GSK3-inhibitor XXII and a HDAC inhibitor that is VPA. The PF-06821497 is at a concentration of about 0.001 μM to 1,000 mM, about 0.01 μM to 100,000 μM, about 0.1 μM to 10,000 μM, about 1 μM to 1,000 μM, about 1 μM to 10 μM, about 10 μM to 100 μM, about 100 μM to 1 mM, or about 1 mM to 10 mM. and the GSK3-inhibitor XXII, at a concentration of about of about 0.1 μM to 1,000 mM, about 1 μM to 100 mM, about 10 μM to 10 mM, about 0.1 μM to 1 μM, about 1 μM to 10 μM, about 10 μM to 100 μM, about 100 μM to 1 μM, about 1 mM to 10 mM, about 10 mM to 100 mM, or about 100 mM to 1000 mM and the VPA at a concentration about 100 mM to 4,000 mM.

In some embodiments, the PF-06821497 at a concentration of about 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, 10 mM, 12 mM, 14 mM, 16 mM, 18 mM, 20 mM, 25 mM, or about 30 mM and the GSK3-inhibitor XXII is at a concentration of about 0.1 mM, 0.2 mM, 0.3 mM, 0.4 mM, 0.5 mM, 0.6 mM, 0.7 mM, 0.8 mM, 0.9 mM, or 1.0 mM. and the VPA at a concentration about 100 mM to 4,000 mM.

In some embodiments, the pharmaceutical composition comprises an EZH2 inhibitor that is PF-06821497 and a GSK3 Inhibitor that is CHIR99021 and a HDAC inhibitor that is VPA. The PF-06821497 is at a concentration of about 0.001 μM to 1,000 mM, about 0.01 μM to 100,000 μM, about 0.1 μM to 10,000 μM, about 1 μM to 1,000 μM, about 1 μM to 10 μM, about 10 μM to 100 μM, about 100 μM to 1 mM, or about 1 mM to 10 mM. and the CHIR99021 at a concentration of about 0.001 mM to 10,000 mM, about 0.01 mM to 1,000 mM, about 0.1 mM to 100 mM, about 0.001 mM to 0.01 mM, about 0.01 mM to 0.1 mM, about 0.1 mM to 1 mM, about 1 mM to 10 mM, about 10 mM to 100 mM, about 100 mM to 1,000 mM, or about 1,000 mM to 10,000 mM and the VPA at a concentration about 100 mM to 4,000 mM.

In some embodiments, the PF-06821497 at a concentration of about 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, 10 mM, 12 mM, 14 mM, 16 mM, 18 mM, 20 mM, 25 mM, or about 30 mM and the CHIR99021 is at a concentration of about 1 mM, 2 mM, 3 mM, 4 mM, 5 mM, 6 mM, 7 mM, 8 mM, 9 mM, or 10 mM and the VPA at a concentration about 100 mM to 4,000 mM.

In some embodiments, the pharmaceutical composition comprises an EZH2 inhibitor that is CPI-360 and a GSK3 Inhibitor that is AZD1080 and a HDAC inhibitor that is VPA. The CPI-360 is at a concentration of about 0.001 μM to 1,000 mM about 0.01 μM to 100,000 μM, about 0.1 μM to 10,000 μM, about 1 μM to 1,000 μM, about 1 μM to 10 μM, about 10 μM to 100 μM, about 100 μM to 1 mM, or about 1 mM to 10 mM. and the AZ1090 is at a concentration of about 0.001 mM to 10,000 mM, about 0.01 mM to 1,000 mM, about 0.1 mM to 100 mM, about 0.001 mM to 0.01 mM, about 0.01 mM to 0.1 mM, about 0.1 mM to 1 mM, about 1 mM to 10 mM, about 10 mM to 100 mM, about 100 mM to 1,000 mM, or about 1,000 mM to 10,000 mM and the VPA at a concentration about 100 mM to 4,000 mM.

In some embodiments, the CPI-360 at a concentration of about 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, 10 mM, 12 mM, 14 mM, 16 mM, 18 mM, 20 mM, 25 mM, or about 30 mM and the AZ1090 is at a a concentration of about 1 mM, 2 mM, 3 mM, 4 mM, 5 mM, 6 mM, 7 mM, 8 mM, 9 mM, or 10 mM and the VPA at a concentration about 100 mM to 4,000 mM.

In some embodiments, the pharmaceutical composition comprises an EZH2 inhibitor that is CPI-360 and a GSK3 Inhibitor that is LY2090314 and a HDAC inhibitor that is VPA. The CPI-360 is at a concentration of about 0.001 μM to 1,000 mM, about 0.01 μM to 100,000 μM, about 0.1 μM to 10,000 μM, about 1 μM to 1,000 μM, about 1 μM to 10 μM, about 10 μM to 100 μM, about 100 μM to 1 mM, or about 1 mM to 10 mM. and the LY2090314 at a concentration of about 0.001 μM to 10 mM, about 0.01 μM to 1 mM, about 0.1 μM to 100 uM, about 0.001 μM to 0.01 μM, about 0.01 μM to 0.1 μM, about 0.1 μM to 1 μM about 1 μM to 10 μM, about 10 μM to 100 μM, about 100 μM to 1 mM, or about 1 mM to 10 mM and the VPA at a concentration about 100 mM to 4,000 mM.

In some embodiments, the CPI-360 at a concentration of about 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, 10 mM, 12 mM, 14 mM, 16 mM, 18 mM, 20 mM, 25 mM, or about 30 mM and the LY2090314 the is at a concentration of about 1 μM, 5 μM, 10 μM, 15 μM, 20 μM or 40 μM and the VPA at a concentration about 100 mM to 4,000 mM.

In some embodiments, the pharmaceutical composition comprises an EZH2 inhibitor that is CPI-360 and a GSK3 Inhibitor that is that is a substituted 3-Imidazo[1,2-a]pyridin-3-yl-4-(1,2,3,4-tetrahydro-[1,4]diazepino-[6,7,1-hi]indol-7-yl)pyrrole-2,5-dione and a HDAC inhibitor that is VPA. The CPI-360 is at a concentration of about 0.001 μM to 1,000 mM, about 0.01 μM to 100,000 μM about 0.1 μM to 10,000 μM, about 1 μM to 1,000 μM, about 1 μM to 10 μM, about 10 μM to 100 μM, about 100 μM to 1 mM, or about 1 mM to 10 mM, and the substituted 3-Imidazo[1,2-a]pyridin-3-yl-4-(1,2,3,4-tetrahydro-[1,4]diazepino-[6,7,1-hi]indol-7-yl)pyrrole-2,5-dime at a concentration of about 0.001 μM to 10 mM, about 0.01 μM to 1 mM, about 0.1 μM to 100 uM, about 0.001 μM to 0.01 μM, about 0.01 μM to 0.1 μM, about 0.1 μM to 1 μM, about 1 μM to 10 μM, about 10 μM to 100 μM, about 100 μM to 1 mM, or about 1 mM to 10 mM. and the VPA at a concentration about 100 mM to 4,000 mM.

In some embodiments, the CPI-360 at a concentration of about 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, 10 mM, 12 mM, 14 mM, 16 mM, 18 mM, 20 mM, 25 mM, or about 30 mM and the substituted 3-Imidazo[1,2-a]pyridin-3-yl-4-(1,2,3,4-tetrahydro-[1,4]diazepino-[6,7,1-hi]indol-7-yl)pyrrole-2,5-dione, is at a concentration of about 1 μM, 5 μM, 10 μM, 15 μM, 20 μM, 50 μM, 100 μM, 250 μM or 500 μM and the VPA at a concentration about 100 mM to 4,000 mM.

In some embodiments, the pharmaceutical composition comprises an EZH2 inhibitor that is CPI-360 and a GSK3 Inhibitor that is GSK3-inhibitor XXII and a HDAC inhibitor that is VPA. The CPI-360 is at a concentration of about 0.001 μM to 1,000 mM, about 0.01 μM to 100,000 μM, about 0.1 μM to 10,000 μM, about 1 μM to 1,000 μM, about 1 μM to 10 μM, about 10 μM to 100 μM, about 100 μM to 1 mM, or about 1 mM to 10 mM. and the GSK3-inhibitor XXII, at a concentration of about of about 0.1 μM to 1,000 mM, about 1 μM to 100 mM, about 10 μM to 10 mM, about 0.1 μM to 1 μM, about 1 μM to 10 μM, about 10 μM to 100 μM, about 100 μM to 1 mM, about 1 mM to 10 mM, about 10 mM to 100 mM, or about 100 mM to 1000 mM and the VPA at a concentration about 100 mM to 4,000 mM.

In some embodiments, the CPI-360 at a concentration of about 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, 10 mM, 12 mM, 14 mM, 16 mM, 18 mM, 20 mM, 25 mM, or about 30 mM and the GSK3-inhibitor XXII is at a concentration of about 0.1 mM, 0.2 mM, 0.3 mM, 0.4 mM, 0.5 mM, 0.6 mM, 0.7 mM, 0.8 mM, 0.9 mM, or 1.0 mM. and the VPA at a concentration about 100 mM to 4,000 mM.

In some embodiments, the pharmaceutical composition comprises an EZH2 inhibitor that is CPI-360 and a GSK3 Inhibitor that is CHIR99021 and a HDAC inhibitor that is VPA. The CPI-360 is at a concentration of about 0.001 μM to 1,000 mM, about 0.01 μM to 100,000 μM, about 0.1 μM to 10,000 μM, about 1 μM to 1,000 μM, about 1 μM to 10 μM, about 10 μM to 100 μM, about 100 μM to 1 mM, or about 1 mM to 10 mM, and the CHIR99021 at a concentration of about 0.001 mM to 10,000 mM, about 0.01 mM to 1,000 mM, about 0.1 mM to 100 mM, about 0.001 mM to 0.01 mM, about 0.01 mM to 0.1 mM, about 0.1 mM to 1 mM, about 1 mM to 10 mM, about 10 mM to 100 mM, about 100 mM to 1,000 mM, or about 1,000 mM to 10,000 mM and the VPA at a concentration about 100 mM to 4,000 mM.

In some embodiments, the CPI-360 at a concentration of about 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, 10 mM, 12 mM, 14 mM, 16 mM, 18 mM, 20 mM, 25 mM, or about 30 mM and the CHIR99021 is at a concentration of about 1 mM, 2 mM, 3 mM, 4 mM, 5 mM, 6 mM, 7 mM, 8 mM, 9 mM, or 10 mM and the VPA at a concentration about 100 mM to 4,000 mM.

In some embodiments, the pharmaceutical composition comprises an EZH2 inhibitor that is EPZ011989 and a GSK3 Inhibitor that is AZD1080 and a HDAC inhibitor that is VPA. The EPZ011989 is at a concentration of about 0.001 μM to 1,000 mM, about 0.01 μM to 100,000 μM, about 0.1 μM to 10,000 μM, about 1 μM to 1,000 μM, about 1 μM to 10 μM, about 10 μM to 100 μM, about 100 μM to 1 mM, or about 1 mM to 10 mM. and the AZ1090 is at a concentration of about 0.001 mM to 10,000 mM, about 0.01 mM to 1,000 mM, about 0.1 mM to 100 mM, about 0.001 mM to 0.01 mM, about 0.01 mM to 0.1 mM, about 0.1 mM to 1 mM, about 1 mM to 10 mM, about 10 mM to 100 mM, about 100 mM to 1,000 mM, or about 1,000 mM to 10,000 mM and the VPA at a concentration about 100 mM to 4,000 mM.

In some embodiments, the EPZ011989 at a concentration of about 0.1 μM, 0.2 μM, 0.3 μM, 0.4 μM, 0.5 μM, 0.6 μM,0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, 10 mM, 12 mM, 14 mM, 16 mM, 18 mM, 20 mM, 25 mM, or about 30 mM and the AZ1090 is at a a concentration of about 1 mM, 2 mM, 3 mM, 4 mM, 5 mM, 6 mM, 7 mM, 8 mM, 9 mM, or 10 mM and the VPA at a concentration about 100 mM to 4,000 mM.

In some embodiments, the pharmaceutical composition comprises an EZH2 inhibitor that is EPZ011989 and a GSK3 Inhibitor that is LY2090314 and a HDAC inhibitor that is VPA. The EPZ011989 is at a concentration of about 0.001 μM to 1,000 mM, about 0.01 μM to 100,000 μM, about 0.1 μM to 10,000 μM, about 1 μM to 1,000 μM, about 1 μM to 10 μM, about 10 μM to 100 μM, about 100 μM to 1 mM, or about 1 mM to 10 mM. and the LY2090314 at a concentration of about 0.001 μM to 10 mM, about 0.01 μM to 1 mM, about 0.1 μM to 100 uM, about 0.001 μM to 0.01 μM, about 0.01 μM to 0.1 μM, about 0.1 μM to 1 μM, about 1 μM to 10 μM, about 10 μM to 100 μM, about 100 μM to 1 mM, or about 1 mM to 10 mM and the VPA at a concentration about 100 mM to 4,000 mM.

In some embodiments, the EPZ011989 at a concentration of about 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, 10 mM, 12 mM, 14 mM, 16 mM, 18 mM, 20 mM, 25 mM, or about 30 mM and the LY2090314 the is at a concentration of about 1 μM, 5 μM, 10 μM, 15 μM, 20 μM or 40 μM and the VPA at a concentration about 100 mM to 4,000 mM.

In some embodiments, the pharmaceutical composition comprises an EZH2 inhibitor that is EPZ011989 and a GSK3 Inhibitor that is that is a substituted 3-Imidazo[1,2-a]pyridin-3-yl-4-(1,2,3,4-tetrahydro-[1,4]diazepino-[6,7,1-hi]indol-7-yl)pyrrole-2,5-dione and a HDAC inhibitor that is VPA. The EPZ011989 is at a concentration of about 0.001 μM to 1,000 mM, about 0.01 μM to 100,000 μM, about 0.1 μM to 10,000 μM, about 1 μM to 1,000 μM, about 1 μM to 10 μM, about 10 μM to 100 μM, about 100 μM to 1 mM, or about 1 mM to 10 mM. and the substituted 3-Imidazo[1,2-a]pyridin-3-yl-4-(1,2,3,4-tetrahydro-[1,4]diazepino-[6,7,1-hi]indol-7-yl)pyrrole-2,5-dione at a concentration of about 0.001 μM to 10 mM, about 0.01 μM to 1 mM, about 0.1 μM to 100 uM, about 0.001 μM to 001 μM, about 0.01 μM to 0.1 μM, about 0.1 μM to 1 μM, about 1 μM to 10 μM, about 10 μM to 100 μM, about 100 μM to 1 mM, or about 1 mM to 10 mM. and the VPA at a concentration about 100 mM to 4,000 mM.

In some embodiments, the EPZ011989 at a concentration of about 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, 10 mM, 12 mM, 14 mM, 16 mM, 18 mM, 20 mM, 25 mM, or about 30 mM and the substituted 3-Imidazo[1,2-a]pyridin-3-yl-4-(1,2,3,4-tetrahydro-[1,4]diazepino-[6,7,1-hi]indol-7-yl)pyrrole-2,5-dione, is at a concentration of about 1 μM, 5 μM, 10 μM, 15 μM, 20 μM, 50 μM, 100 μM, 250 μM or 500 μM and the VPA at a concentration about 100 mM to 4,000 mM.

In some embodiments, the pharmaceutical composition comprises an EZH2 inhibitor that is EPZ011989 and a GSK3 Inhibitor that is GSK3-inhibitor XXII and a HDAC inhibitor that is VPA. The EPZ011989 is at a concentration of about 0.001 μM to 1,000 mM, about 0.01 μM to 100,000 μM, about 0.1 μM to 10,000 μM, about 1 μM to 1,000 μM, about 1 μM to 10 μM, about 10 μM to 100 μM, about 100 μM to 1 mM, or about 1 mM to 10 mM. and the GSK3-inhibitor XXII, at a concentration of about of about 0.1 μM to 1,000 mM, about 1 μM to 100 mM, about 10 μM to 10 mM, about 0.1 μM to 1 μM about 1 μM to 10 μM, about 10 μM to 100 μM, about 100 μM to 1 mM, about 1 mM to 10 mM, about 10 mM to 100 mM, or about 100 mM to 1000 mM and the VPA at a concentration about 100 mM to 4,000 mM.

In some embodiments, the EPZ011989 at a concentration of about 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, 10 mM, 12 mM, 14 mM, 16 mM, 18 mM, 20 mM, 25 mM, or about 30 mM and the GSK3-inhibitor XXII is at a concentration of about 0.1 mM, 0.2 mM, 0.3 mM, 0.4 mM, 0.5 mM, 0.6 mM, 0.7 mM, 0.8 mM, 0.9 mM, or 1.0 mM. and the VPA at a concentration about 100 mM to 4,000 mM.

In some embodiments, the pharmaceutical composition comprises an EZH2 inhibitor that is EPZ011989 and a GSK3 Inhibitor that is CHIR99021 and a HDAC inhibitor that is VPA. The EPZ011989 is at a concentration of about 0.001 μM to 1,000 mM, about 0.01 μM to 100,000 μM, about 0.1 μM to 10,000 μM, about 1 μM to 1,000 μM, about 1 μM to 10 μM, about 10 μM to 100 μM, about 100 μM to 1 mM, or about 1 mM to 10 mM, and the CHIR99021 at a concentration of about 0.001 mM to 10,000 mM, about 0.01 mM to 1,000 mM, about 0.1 mM to 100 mM, about 0.001 mM to 0.01 mM, about 0.01 mM to 0.1 mM, about 0.1 mM to 1 mM, about 1 mM to 10 mM, about 10 mM to 100 mM, about 100 mM to 1,000 mM, or about 1,000 mM to 10,000 mM and the VPA at a concentration about 100 mM to 4,000 mM.

In some embodiments, the EPZ011989 at a concentration of about 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, 10 mM, 12 mM, 14 mM, 16 mM, 18 mM, 20 mM, 25 mM, or about 30 mM and the CHIR99021 is at a concentration of about 1 mM, 2 mM, 3 mM, 4 mM, 5 mM, 6 mM, 7 mM, 8 mM, 9 mM, or 10 mM and the VPA at a concentration about 100 mM to 4,000 mM.

In some embodiments, the pharmaceutical composition comprises an EZH2 inhibitor that is UNC 2399 and a GSK3 Inhibitor that is AZD1080 and a HDAC inhibitor that is VPA. The UNC 2399 is at a concentration of about 0.001 μM to 1,000 mM, about 0.01 μM to 100,000 μM, about 0.1 μM to 10,000 μM, about 1 μM to 1,000 μM, about 1 μM to 10 μM, about 10 μM to 100 μM, about 100 μM to 1 mM, or about 1 mM to 10 mM. and the AZ1090 is at a concentration of about 0.001 mM to 10,000 mM, about 0.01 mM to 1,000 mM, about 0.1 mM to 100 mM, about 0.001 mM to 0.01 mM, about 0.01 mM to 0.1 mM, about 0.1 mM to 1 mM, about 1 mM to 10 mM, about 10 mM to 100 mM, about 100 mM to 1,000 mM, or about 1,000 mM to 10,000 mM and the VPA at a concentration about 100 mM to 4,000 mM.

In some embodiments, the UNC 2399 at a concentration of about 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, 10 mM, 12 mM, 14 mM, 16 mM, 18 mM, 20 mM, 25 mM, or about 30 mM and the AZ1090 is at a a concentration of about 1 mM, 2 mM, 3 mM, 4 mM, 5 mM, 6 mM, 7 mM, 8 mM, 9 mM, or 10 mM and the VPA at a concentration about 100 mM to 4,000 mM.

In some embodiments, the pharmaceutical composition comprises an EZH2 inhibitor that is UNC 2399 and a GSK3 Inhibitor that is LY2090314 and a HDAC inhibitor that is VPA. The UNC 2399 is at a concentration of about 0.001 μM to 1,000 mM, about 0.01 μM to 100,000 μM, about 0.1 μM to 10,000 μM, about 1 μM to 1,000 μM, about 1 μM to 10 μM, about 10 μM to 100 μM, about 100 μM to 1 mM, or about 1 mM to 10 mM. and the LY2090314 at a concentration of about 0.001 μM to 10 mM, about 0.01 μM to 1 mM, about 0.1 μM to 100 uM, about 0.001 μM to 0.01 μM, about 0.01 μM to 0.1 μM, about 0.1 μM to 1 μM, about 1 μM to 10 μM, about 10 μM to 100 μM, about 100 μM to 1 mM, or about 1 mM to 10 mM and the VPA at a concentration about 100 mM to 4,000 mM.

In some embodiments, the UNC 2399 at a concentration of about 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, 10 mM, 12 mM, 14 mM, 16 mM, 18 mM, 20 mM, 25 mM, or about 30 mM and the LY2090314 the is at a concentration of about 1 μM, 5 μM, 10 μM, 15 μM, 20 μM or 40 μM and the VPA at a concentration about 100 mM to 4,000 mM.

In some embodiments, the pharmaceutical composition comprises an EZH2 inhibitor that is UNC 2399 and a GSK3 Inhibitor that is that is a substituted 3-Imidazo[1,2-a]pyridin-3-yl-4-(1,2,3,4-tetrahydro-[1,4]diazepino-[6,7,1-hi]indol-7-yl)pyrrole-2,5-dione and a HDAC inhibitor that is VPA. The UNC 2399 is at a concentration of about 0.001 μM to 1,000 mM, about 0.01 μM to 100,000 μM, about 0.1 μM to 10,000 μM, about 1 μM to 1,000 μM, about 1 μM to 10 μM, about 10 μM to 100 μM, about 100 μM to 1 mM, or about 1 mM to 10 mM. and the substituted 3-Imidazo[1,2-a]pyridin-3-yl-4-(1,2,3,4-tetrahydro-[1,4]diazepino-[6,7,1-hi]indol-7-yl)pyrrole-2,5-dione at a concentration of about 0.001 μM to 10 mM, about 0.01 μM to 1 mM, about 0.1 μM to 100 uM, about 0.001 μM to 0.01 μM, about 0.01 μM to 0.1 μM, about 0.1 μM to 1 μM, about 1 μM to 10 μM, about 10 μM to 100 μM, about 100 μM to 1 mM, or about 1 mM to 10 mM and the VPA at a concentration about 100 mM to 4,000 mM.

In some embodiments, the UNC 2399 at a concentration of about 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, 10 mM, 12 mM, 14 mM, 16 mM, 18 mM, 20 mM, 25 mM, or about 30 mM and the substituted 3-Imidazo[1,2-a]pyridin-3-yl-4-(1,2,3,4-tetrahydro-[1,4]diazepino-[6,7,1-hi]indol-7-yl)pyrrole-2,5-dione, is at a concentration of about 1 μM, 5 μM, 10 μM, 15 μM, 20 μM, 50 μM, 100 μM, 250 μM or 500 μM and the VPA at a concentration about 100 mM to 4,000 mM.

In some embodiments, the pharmaceutical composition comprises an EZH2 inhibitor that is UNC 2399 and a GSK3 Inhibitor that is GSK3-inhibitor XXII and a HDAC inhibitor that is VPA. The UNC 2399 is at a concentration of about 0.001 μM to 1,000 mM, about 0.01 μM to 100,000 μM, about 0.1 μM to 10,000 μM, about 1 μM to 1,000 μM, about 1 μM to 10 μM, about 10 μM to 100 μM, about 100 μM to 1 mM, or about 1 mM to 10 mM. and the GSK3-inhibitor XXII, at a concentration of about of about 0.1 μM to 1,000 mM, about 1 μM to 100 mM, about 10 μM to 10 mM, about 0.1 μM to 1 μM, about 1 μM to 10 μM, about 10 μM to 100 μM, about 100 μM to 1 mM, about 1 mM to 10 mM, about 10 mM to 100 mM, or about 100 mM to 1000 mM and the VPA at a concentration about 100 mM to 4,000 mM.

In some embodiments, the UNC 2399 at a concentration of about 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, 10 mM, 12 mM, 14 mM, 16 mM, 18 mM, 20 mM, 25 mM, or about 30 mM and the GSK3-inhibitor XXII is at a concentration of about 0.1 mM, 0.2 mM, 0.3 mM, 0.4 mM, 0.5 mM, 0.6 mM, 0.7 mM, 0.8 mM, 0.9 mM, or 1.0 mM and the VPA at a concentration about 100 mM to 4,000 mM.

In some embodiments, the pharmaceutical composition comprises an EZH2 inhibitor that is UNC 2399 and a GSK3 Inhibitor that is CHIR99021 and a HDAC inhibitor that is VPA. The UNC 2399 is at a concentration of about 0.001 μM to 1,000 mM, about 0.01 μM to 100,000 μM, about 0.1 μM to 10,000 μM, about 1 μM to 1,000 μM, about 1 μM to 10 μM, about 10 μM to 100 μM, about 100 μM to 1 mM, or about 1 mM to 10 mM, and the CHIR99021 at a concentration of about 0.001 mM to 10,000 mM, about 0.01 mM to 1,000 mM, about 0.1 mM to 100 mM, about 0.001 mM to 0.01 mM, about 0.01 mM to 0.1 mM, about 0.1 mM to 1 mM, about 1 mM to 10 mM, about 10 mM to 100 mM, about 100 mM to 1,000 mM, or about 1,000 mM to 10,000 mM and the VPA at a concentration about 100 mM to 4,000 mM.

In some embodiments, the UNC 2399 at a concentration of about 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, 10 mM, 12 mM, 14 mM, 16 mM, 18 mM, 20 mM, 25 mM, or about 30 mM and the CHIR99021 is at a concentration of about 1 mM, 2 mM, 3 mM, 4 mM, 5 mM, 6 mM, 7 mM, 8 mM, 9 mM, or 10 mM and the VPA at a concentration about 100 mM to 4,000 mM.

In some embodiments, the pharmaceutical composition comprises an EZH2 inhibitor that is PF-06726304 and a GSK3 Inhibitor that is AZD1080 and a HDAC inhibitor that is VPA. The PF-06726304 is at a concentration of about 0.001 μM to 1,000 mM, about 0.01 μM to 100,000 μM, about 0.1 μM to 10,000 μM, about 1 μM to 1,000 μM, about 1 μM to 10 μM, about 10 μM to 100 μM, about 100 μM to 1 mM, or about 1 mM to 10 mM. and the AZ1090 is at a concentration of about 0.001 mM to 10,000 mM, about 0.01 mM to 1,000 mM, about 0.1 mM to 100 mM, about 0.001 mM to 0.01 mM, about 0.01 mM to 0.1 mM, about 0.1 mM to 1 mM, about 1 mM to 10 mM, about 10 mM to 100 mM, about 100 mM to 1,000 mM, or about 1,000 mM to 10,000 mM and the VPA at a concentration about 100 mM to 4,000 mM.

In some embodiments, the PF-06726304 at a concentration of about 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, 10 mM, 12 mM, 14 mM, 16 mM, 18 mM, 20 mM, 25 mM, or about 30 mM and the AZ1090 is at a a concentration of about 1 mM, 2 mM, 3 mM, 4 mM, 5 mM, 6 mM, 7 mM, 8 mM, 9 mM, or 10 mM and the VPA at a concentration about 100 mM to 4,000 mM.

In some embodiments, the pharmaceutical composition comprises an EZH2 inhibitor that is PF-06726304 and a GSK3 Inhibitor that is LY2090314 and a HDAC inhibitor that is VPA. The PF-06726304 is at a concentration of about 0.001 μM to 1,000 mM, about 0.01 μM to 100,000 μM, about 0.1 μM to 10,000 μM, about 1 μM to 1,000 μM, about 1 μM to 10 μM, about 10 μM to 100 μM, about 100 μM to 1 mM, or about 1 mM to 10 mM. and the LY2090314 at a concentration of about 0.001 μM to 10 mM, about 0.01 μM to 1 mM, about 0.1 μM to 100 uM, about 0.001 μM to 0.01 μM, about 0.01 μM to 0.1 μM, about 0.1 μM to 1 μM, about 1 μM to 10 μM, about 10 μM to 100 μM, about 100 μM to 1 mM, or about 1 mM to 10 mM and the VPA at a concentration about 100 mM to 4,000 mM.

In some embodiments, the PF-06726304 at a concentration of about 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, 10 mM, 12 mM, 14 mM, 16 mM, 18 mM, 20 mM, 25 mM, or about 30 mM and the LY2090314 the is at a concentration of about 1 μM, 5 μM, 10 μM, 15 μM, 20 μM or 40 μM and the VPA at a concentration about 100 mM to 4,000 mM.

In some embodiments, the pharmaceutical composition comprises an EZH2 inhibitor that is PF-06726304 and a GSK3 Inhibitor that is that is a substituted 3-Imidazo[1,2-a]pyridin-3-yl-4-(1,2,3,4-tetrahydro-[1,4]diazepino-[6,7,1-hi]indol-7-yl)pyrrole-2,5-dione and a HDAC inhibitor that is VPA. The PF-06726304 is at a concentration of about 0.001 μM to 1,000 mM, about 0.01 μM to 100,000 μM, about 0.1 μM to 10,000 μM, about 1 μM to 1,000 μM, about 1 μM to 10 μM, about 10 μM to 100 μM, about 100 μM to 1 mM, or about 1 mM to 10 mM. and the substituted 3-Imidazo[1,2-a]pyridin-3-yl-4-(1,2,3,4-tetrahydro-[1,4]diazepino-[6,7,1-hi]indol-7-yl)pyrrole-2,5-dione at a concentration of about 0.001 μM to 10 mM, about 0.01 μM to 1 mM, about 0.1 μM to 100 uM, about 0.001 μM to 0.01 μM, about 0.01 μM to 0.1 μM, about 0.1 μM to 1 μM, about 1 μM to 10 μM, about 10 μM to 100 μM, about 100 μM to 1 mM, or about 1 mM to 10 mM and the VPA at a concentration about 100 mM to 4,000 mM.

In some embodiments, the PF-06726304 at a concentration of about 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, 10 mM, 12 mM, 14 mM, 16 mM, 18 mM, 20 mM, 25 mM, or about 30 mM and the substituted 3-Imidazo[1,2-a]pyridin-3-yl-4-(1,2,3,4-tetrahydro-[1,4]diazepino-[6,7,1-hi]indol-7-yl)pyrrole-2,5-dione, is at a concentration of about 1 μM, 5 μM, 10 μM, 15 μM, 20 μM, 50 μM, 100 μM, 250 μM or 500 μM and the VPA at a concentration about 100 mM to 4,000 mM.

In some embodiments, the pharmaceutical composition comprises an EZH2 inhibitor that is PF-06726304 and a GSK3 Inhibitor that is GSK3-inhibitor XXII and a HDAC inhibitor that is VPA. The PF-06726304 is at a concentration of about 0.001 μM to 1,000 mM, about 0.01 μM to 100,000 μM, about 0.1 μM to 10,000 μM, about 1 μM to 1,000 μM, about 1 μM to 10 μM, about 10 μM to 100 μM, about 100 μM to 1 mM, or about 1 mM to 10 mM. and the GSK3-inhibitor XXII, at a concentration of about of about 0.1 μM to 1,000 mM, about 1 μM to 100 mM, about 10 μM to 10 mM, about 0.1 μM to 1 μM, about 1 μM to 10 μM, about 10 μM to 100 μM, about 100 μM to 1 mM, about 1 mM to 10 mM, about 10 mM to 100 mM, or about 100 mM to 1000 mM and the VPA at a concentration about 100 mM to 4,000 mM.

In some embodiments, the PF-06726304 at a concentration of about 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, 10 mM, 12 mM, 14 mM, 16 mM, 18 mM, 20 mM, 25 mM, or about 30 mM and the GSK3-inhibitor XXII is at a concentration of about 0.1 mM, 0.2 mM, 0.3 mM, 0.4 mM, 0.5 mM, 0.6 mM, 0.7 mM, 0.8 mM, 0.9 mM, or 1.0 mM and the VPA at a concentration about 100 mM to 4,000 mM.

In some embodiments, the pharmaceutical composition comprises an EZH2 inhibitor that is PF-06726304 and a GSK3 Inhibitor that is CHIR99021 and a HDAC inhibitor that is VPA. The PF-06726304 is at a concentration of about 0.001 μM to 1,000 mM, about 0.01 μM to 100,000 μM, about 0.1 μM to 10,000 μM, about 1 μM to 1,000 μM, about 1 μM to 10 μM, about 10 μM to 100 μM, about 100 μM to 1 mM, or about 1 mM to 10 mM. and the CRIR99021 at a concentration of about 0.001 mM to 10,000 mM, about 0.01 mM to 1,000 mM, about 0.1 mM to 100 mM, about 0.001 mM to 0.01 mM, about 0.01 mM to 0.1 mM, about 0.1 mM to 1 mM, about 1 mM to 10 mM, about 10 mM to 100 mM, about 100 mM to 1,000 mM, or about 1,000 mM to 10,000 mM and the VPA at a concentration about 100 mM to 4,000 mM.

In some embodiments, the PF-06726304 at a concentration of about 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, 10 mM, 12 mM, 14 mM, 16 mM, 18 mM, 20 mM, 25 mM, or about 30 mM and the CHIR99021 is at a concentration of about 1 mM, 2 mM, 3 mM, 4 mM, 5 mM, 6 mM, 7 mM, 8 mM, 9 mM, or 10 mM and the VPA at a concentration about 100 mM to 4,000 mM.

In some embodiments, the pharmaceutical composition comprises a DOT1L inhibitor that is EPZ004777and a GSK3 Inhibitor that is AZD1080. The EPZ004777 is in a concentration of about 10 nM, 20 nM, 30 nM, 40 nM, 50 nM, 60 nM, 70 nM, 80 nM, 90 nM, 100 nM, 200 nM, 300 nM, 400 nM, 500 nM, 600 nM, 700 nM, 800 nM, 900 nM, 1 μM, 2 μM, 3 μM, 4 μM, 5 μM, 6 μM, 7 μM, 8 μM, 9 μM, 10 μM, 11 μM, 12 μM, 13 μM, 14 μM, 15 μM, 20 μM, 25 μM, 30 μM, 35 μM, 40 μM, 45 μM, or about 50 μM and the AZ1090 is at a concentration of about 0.001 mM to 10,000 mM, about 0.01 mM to 1,000 mM, about 0.1 mM to 100 mM, about 0.001 mM to 0.01 mM, about 0.01 mM to 0.1 mM, about 0.1 mM to 1 mM, about 1 mM to 10 mM, about 10 mM to 100 mM, about 100 mM to 1,000 mM, or about 1,000 mM to 10,000 mM.

In some embodiments, the EPZ004777 is in a concentration of about 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, 10 mM, 11 mM, 12 mM, 13 mM, 14 mM, 15 mM, 20 mM, 25 mM, 30 mM, 35 mM, 40 mM, 45 mM, or about 50 mM and the AZ1090 is at a a concentration of about 1 mM, 2 mM, 3 mM, 4 mM, 5 mM, 6 mM, 7 mM, 8 mM, 9 mM, or 10 mM.

In some embodiments, the pharmaceutical composition comprises a DOT1L inhibitor that is EPZ004777 and a GSK3 Inhibitor that is LY2090314. The EPZ004777 is in a concentration of about 10 nM, 20 nM, 30 nM, 40 nM, 50 nM, 60 nM, 70 nM, 80 nM, 90 nM, 100 nM, 200 nM, 300 nM, 400 nM, 500 nM, 600 nM, 700 nM, 800 nM, 900 nM, 1 μM, 2 μM, 3 μM, 4 μM, 5 μM, 6 μM, 7 μM, 8 μM, 9 μM, 10 μM, 11 μM, 12 μM, 13 μM, 14 μM, 15 μM, 20 μM, 25 μM, 30 μM, 35 μM, 40 μM, 45 μM, or about 50 μM and the LY2090314 at a concentration of about 0.001 μM to 10 mM, about 0.01 μM to 1 mM, about 0.1 μM to 100 uM, about 0.001 μM to 0.01 μM, about 0.01 μM to 0.1 μM, about 0.1 μM to 1 μM, about 1 μM to 10 μM, about 10 μM to 100 μM, about 100 μM to 1 mM, or about 1 mM to 10 mM.

In some embodiments, the EPZ004777 is in a concentration of about 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, 10 mM, 12 mM, 14 mM, 16 mM, 18 mM, 20 mM, 25 mM, or about 30 mM and the LY2090314 is at a concentration of about 1 μM, 5 μM, 10 μM, 15 μM, 20 μM, or 40 μM.

In some embodiments, the pharmaceutical composition comprises a DOT1L inhibitor that is EPZ004777 and a GSK3 Inhibitor that is that is a substituted 3-Imidazo[1,2-a]pyridin-3-yl-4-(1,2,3,4-tetrahydro-[1,4]diazepino-[6,7,1-hi]indol-7-yl)pyrrole-2,5-dione. The EPZ004777 is in a concentration of about 10 nM, 20 nM, 30 nM, 40 nM, 50 nM, 60 nM, 70 nM, 80 nM, 90 nM, 100 nM, 200 nM, 300 nM, 400 nM, 500 nM, 600 nM, 700 nM, 800 nM, 900 nM, 1 μM, 2 μM, 3 μM, 4 μM, 5 μM, 6 μM, 7 μM, 8 μM, 9 μM, 10 μM, 11 μM, 12 μM, 13 μM, 14 μM, 15 μM, 20 μM, 25 μM, 30 μM, 35 μM, 40 μM, 45 μM, or about 50 μM and the substituted 3-Imidazo[1,2-a]pyridin-3-yl-4-(1,2,3,4-tetrahydro-[1,4]diazepino-[6,7,1-hi]indol-7-yl)pyrrole-2,5-dione at a concentration of about 0.001 μM to 10 mM, about 0.01 μM to 1 mM, about 0.1 μM to 100 uM, about 0.001 μM to 0.01 μM, about 0.01 μM to 0.1 μM, about 0.1 μM to 1 μM, about 1 μM to 10 μM, about 10 μM to 100 μM, about 100 μM to 1 mM, or about 1 mM to 10 mM.

In some embodiments, the EPZ004777 is in a concentration of about 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, 10 mM, 12 mM, 14 mM, 16 mM, 18 mM, 20 mM, 25 mM, or about 30 mM and the substituted 3-Imidazo[1,2-a]pyridin-3-yl-4-(1,2,3,4-tetrahydro-[1,4]diazepino-[6,7,1-hi]indol-7-yl)pyrrole-2,5-dione, is at a concentration of about 1 μM, 5 μM, 10 μM, 15 μM, 20 μM, 50 μM, 100 μM, 250 μM or 500 μM.

In some embodiments, the pharmaceutical composition comprises a DOT1L inhibitor that is EPZ004777 and a GSK3 Inhibitor that is GSK3-inhibitor XXII. The EPZ004777 is in a concentration of about 10 nM, 20 nM, 30 nM, 40 nM, 50 nM, 60 nM, 70 nM, 80 nM, 90 nM, 100 nM, 200 nM, 300 nM, 400 nM, 500 nM, 600 nM, 700 nM, 800 nM, 900 nM, 1 μM, 2 μM, 3 μM, 4 μM, 5 μM, 6 μM, 7 μM, 8 μM, 9 μM, 10 μM, 11 μM, 12 μM, 13 μM, 14 μM, 15 μM, 20 μM, 25 μM, 30 μM, 35 μM, 40 μM, 45 μM, or about 50 μM and the GSK3-inhibitor XXII, at a concentration of about0.1 μM to 1,000 mM, about 1 μM to 100 mM, about 10 μM to 10 mM, about 0.1 μM to 1 μM, about 1 μM to 10 μM, about 10 μM to 100 μM, about 100 μM to 1 mM, about 1 mM to 10 mM, about 10 mM to 100 mM, or about 100 mM to 1000 mM.

In some embodiments, the EPZ004777 is in a concentration of about 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, 10 mM, 12 mM, 14 mM, 16 mM, 18 mM, 20 mM, 25 mM, or about 30 mM and the GSK3-inhibitor XXII is at a concentration of about 0.1 mM, 0.2 mM, 0.3 mM, 0.4 mM, 0.5 mM, 0.6 mM, 0.7 mM, 0.8 mM, 0.9 mM, or 1.0 mM.

In some embodiments, the pharmaceutical composition comprises a DOT1L inhibitor that is EPZ004777 and a GSK3 Inhibitor that is CHIR99021. The EPZ004777 is in a concentration of about 10 nM, 20 nM, 30 nM, 40 nM, 50 nM, 60 nM, 70 nM, 80 nM, 90 nM, 100 nM, 200 nM, 300 nM, 400 nM, 500 nM, 600 nM, 700 nM, 800 nM, 900 nM, 1 μM, 2 μM, 3 μM, 4 μM, 5 μM, 6 μM, 7 μM, 8 μM, 9 μM, 10 μM, 11 μM, 12 μM, 13 μM, 14 μM, 15 μM, 20 μM, 25 μM, 30 μM, 35 μM, 40 μM, 45 μM, or about 50 μM and the CHIR99021 at a concentration of about 0.001 mM to 10,000 mM, about 0.01 mM to 1,000 mM, about 0.1 mM to 100 mM, about 0.001 mM to 0.01 mM, about 0.01 mM to 0.1 mM, about 0.1 mM to 1 mM, about 1 mM to 10 mM, about 10 mM to 100 mM, about 100 mM to 1,000 mM, or about 1,000 mM to 10,000 mM.

In some embodiments, the EPZ004777 is in a concentration of about 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, 10 mM, 12 mM, 14 mM, 16 mM, 18 mM, 20 mM, 25 mM, or about 30 mM and the CHIR99021 is at a concentration of about 1 mM, 2 mM, 3 mM, 4 mM, 5 mM, 6 mM, 7 mM, 8 mM, 9 mM, or 10 mM.

In some embodiments, the pharmaceutical composition comprises a DOT1L inhibitor that is pinometostat and a GSK3 Inhibitor that is AZD1080. The pinometostat is in a concentration of about 10 nM, 20 nM, 30 nM, 40 nM, 50 nM, 60 nM, 70 nM, 80 nM, 90 nM, 100 nM, 200 nM, 300 nM, 400 nM, 500 nM, 600 nM, 700 nM, 800 nM, 900 nM, 1 μM, 2 μM, 3 μM, 4 μM, 5 μM, 6 μM, 7 μM, 8 μM, 9 μM, 10 μM, 11 μM, 12 μM, 13 μM, 14 μM, 15 μM, 20 μM, 25 μM, 30 μM, 35 μM, 40 μM, 45 μM, or about 50 μM and the AZ1090 is at a concentration of about 0.001 mM to 10,000 mM, about 0.01 mM to 1,000 mM, about 0.1 mM to 100 mM, about 0.001 mM to 0.01 mM, about 0.01 mM to 0.1 mM, about 0.1 mM to 1 mM, about 1 mM to 10 mM, about 10 mM to 100 mM, about 100 mM to 1,000 mM, or about 1,000 mM to 10,000 mM.

In some embodiments, the pinometostat is in a concentration of about 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, 10 mM, 11 mM, 12 mM, 13 mM, 14 mM, 15 mM, 20 mM, 25 mM, 30 mM, 35 mM, 40 mM, 45 mM, or about 50 mM and the AZ1090 is at a a concentration of about 1 mM, 2 mM, 3 mM, 4 mM, 5 mM, 6 mM, 7 mM, 8 mM, 9 mM, or 10 mM.

In some embodiments, the pharmaceutical composition comprises a DOT1L inhibitor that is pinometostat and a GSK3 Inhibitor that is LY2090314. The pinometostat is in a concentration of about 10 nM, 20 nM, 30 nM, 40 nM, 50 nM, 60 nM, 70 nM, 80 nM, 90 nM, 100 nM, 200 nM, 300 nM, 400 nM, 500 nM, 600 nM, 700 nM, 800 nM, 900 nM, 1 μM, 2 μM, 3 μM, 4 μM, 5 μM, 6 μM, 7 μM, 8 μM, 9 μM, 10 μM, 11 μM, 12 μM, 13 μM, 14 μM, 15 μM, 20 μM, 25 μM, 30 μM, 35 μM, 40 μM, 45 μM, or about 50 μM and the LY2090314 at a concentration of about 0.001 μM to 10 mM, about 0.01 μM to 1 mM, about 0.1 μM to 100 uM, about 0.001 μM to 0.01 μM, about 0.01 μM to 0.1 μM, about 0.1 μM to 1 μM, about 1 μM to 10 μM, about 10 μM to 100 μM, about 100 μM to 1 mM, or about 1 mM to 10 mM.

In some embodiments, the pinometostat is in a concentration of about 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, 10 mM, 11 mM, 12 mM, 13 mM, 14 mM, 15 mM, 20 mM, 25 mM, 30 mM, 35 mM, 40 mM, 45 mM, or about 50 mM. and the LY2090314 is at a concentration of about 1 μM, 5 μM, 10 μM, 15 μM, 20 μM, or 30 μM.

In some embodiments, the pharmaceutical composition comprises a DOT1L inhibitor that is pinometostat and a GSK3 Inhibitor that is that is a substituted 3-Imidazo[1,2-a]pyridin-3-yl-4-(1,2,3,4-tetrahydro-[1,4]diazepino-[6,7,1-hi]indol-7-yl)pyrrole-2,5-dione. The pinometostat is in a concentration of about 10 nM, 20 nM, 30 nM, 40 nM, 50 nM, 60 nM, 70 nM, 80 nM, 90 nM, 100 nM, 200 nM, 300 nM, 400 nM, 500 nM, 600 nM, 700 nM, 800 nM, 900 nM, 1 μM, 2 μM, 3 μM, 4 μM, 5 μM, 6 μM, 7 μM, 8 μM, 9 μM, 10 μM, 11 μM, 12 μM, 13 μM, 14 μM, 15 μM, 20 μM, 25 μM, 30 μM, 35 μM, 40 μM, 45 μM, or about 50 μM. and the substituted 3-Imidazo[1,2-a]pyridin-3-yl-4-(1,2,3,4-tetrahydro-[1,4]diazepino-[6,7,1-hi]indol-7-yl)pyrrole-2,5-dione at a concentration of about 0.001 μM to 10 mM, about 0.01 μM to 1 mM, about 0.1 μM to 100 uM, about 0.001 μM to 0.01 μM, about 0.01 μM to 0.1 μM, about 0.1 μM to 1 μM, about 1 μM to 10 μM, about 10 μM to 100 μM, about 100 μM to 1 mM, or about 1 mM to 10 mM.

In some embodiments, the pinometostat is in a concentration of about 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, 10 mM, 11 mM, 12 mM, 13 mM, 14 mM, 15 mM, 20 mM, 25 mM, 30 mM, 35 mM, 40 mM, 45 mM, or about 50 mM and the substituted 3-Imidazo[1,2-a]pyridin-3-yl-4-(1,2,3,4-tetrahydro-[1,4]diazepino-[6,7,1-hi]indol-7-yl)pyrrole-2,5-dione, is at a concentration of about 1 μM, 5 μM, 10 μM, 15 μM, 20 μM, 50 μM, 100 μM, 250 μM or 500 μM.

In some embodiments, the pharmaceutical composition comprises a DOT1L inhibitor that is pinometostat and a GSK3 Inhibitor that is GSK3-inhibitor XXII. The pinometostat is in a concentration of about 10 nM, 20 nM, 30 nM, 40 nM, 50 nM, 60 nM, 70 nM, 80 nM, 90 nM, 100 nM, 200 nM, 300 nM, 400 nM, 500 nM, 600 nM, 700 nM, 800 nM, 900 nM, 1 μM, 2 μM, 3 μM, 4 μM, 5 μM, 6 μM, 7 μM, 8 μM, 9 μM, 10 μM, 11 μM, 12 μM, 13 μM, 14 μM, 15 μM, 20 μM, 25 μM, 30 μM, 35 μM, 40 μM, 45 μM, or about 50 μM and the GSK3-inhibitor XXII, at a concentration of about 0.1 μM to 1,000 mM, about 1 μM to 100 mM, about 10 μM to 10 mM, about 0.1 μM to 1 μM, about 1 μM to 10 μM, about 10 μM to 100 μM, about 100 μM to 1 mM, about 1 mM to 10 mM, about 10 mM to 100 mM, or about 100 mM to 1000 mM.

In some embodiments, the pinometostat is in a concentration of about 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, 10 mM, 11 mM, 12 mM, 13 mM, 14 mM, 15 mM, 20 mM, 25 mM, 30 mM, 35 mM, 40 mM, 45 mM, or about 50 mM and the GSK3-inhibitor XXII is at a concentration of about 0.1 mM, 0.2 mM, 0.3 mM, 0.4 mM, 0.5 mM, 0.6 mM, 0.7 mM, 0.8 mM, 0.9 mM or 1.0 mM.

In some embodiments, the pharmaceutical composition comprises a DOT1L inhibitor that is pinometostat and a GSK3 Inhibitor that is CHIR99021. The pinometostat is in a concentration of about 10 nM, 20 nM, 30 nM, 40 nM, 50 nM, 60 nM, 70 nM, 80 nM, 90 nM, 100 nM, 200 nM, 300 nM, 400 nM, 500 nM, 600 nM, 700 nM, 800 nM, 900 nM, 1 μM, 2 μM, 3 μM, 4 μM, 5 μM, 6 μM, 7 μM, 8 μM, 9 μM, 10 μM, 11 μM, 12 μM, 13 μM, 14 μM, 15 μM, 20 μM, 25 μM, 30 μM, 35 μM, 40 μM, 45 μM, or about 50 μM and the CHIR99021 at a concentration of about 0.001 mM to 10,000 mM, about 0.01 mM to 1,000 mM, about 0.1 mM to 100 mM, about 0.001 mM to 0.01 mM, about 0.01 mM to 0.1 mM, about 0.1 mM to 1 mM, about 1 mM to 10 mM, about 10 mM to 100 mM, about 100 mM to 1,000 mM, or about 1,000 mM to 10,000 mM.

In some embodiments, the pinometostat is in a concentration of about 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, 10 mM, 11 mM, 12 mM, 13 mM, 14 mM, 15 mM, 20 mM, 25 mM, 30 mM, 35 mM, 40 mM, 45 mM, or about 50 mM and the CHIR99021 is at a concentration of about 1 mM, 2 mM, 3 mM, 4 mM, 5 mM, 6 mM, 7 mM, 8 mM, 9 mM, or 10 mM.

In some embodiments, the pharmaceutical composition comprises a DOT1L inhibitor that is SGC0946 and a GSK3 Inhibitor that is AZD1080. The SGC0946 is in a concentration of about 10 nM, 20 nM, 30 nM, 40 nM, 50 nM, 60 nM, 70 nM, 80 nM, 90 nM, 100 nM, 200 nM, 300 nM, 400 nM, 500 nM, 600 nM, 700 nM, 800 nM, 900 nM, 1 μM, 2 μM, 3 μM, 4 μM, 5 μM, 6 μM, 7 μM, 8 μM, 9 μM, 10 μM, 11 μM, 12 μM, 13 μM, 14 μM, 15 μM, 20 μM, 25 μM, 30 μM, 35 μM, 40 μM, 45 μM, or about 50 μM and the AZ1090 is at a concentration of about 0.001 mM to 10,000 mM, about 0.01 mM to 1,000 mM, about 0.1 mM to 100 mM, about 0.001 mM to 0.01 mM, about 0.01 mM to 0.1 mM, about 0.1 mM to 1 mM, about 1 mM to 10 mM, about 10 mM to 100 mM, about 100 mM to 1,000 mM, or about 1,000 mM to 10,000 mM.

In some embodiments, the SGC0946 is in a concentration of about 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, 10 mM, 11 mM, 12 mM, 13 mM, 14 mM, 15 mM, 20 mM, 25 mM, 30 mM, 35 mM, 40 mM, 45 mM, or about 50 mM and the AZ1090 is at a a concentration of about 1 mM, 2 mM, 3 mM, 4 mM, 5 mM, 6 mM, 7 mM, 8 mM, 9 mM, or 10 mM.

In some embodiments, the pharmaceutical composition comprises a DOT1L inhibitor that is SGC0946 and a GSK3 Inhibitor that is LY2090314. The SGC0946 is in a concentration of about 10 nM, 20 nM, 30 nM, 40 nM, 50 nM, 60 nM, 70 nM, 80 nM, 90 nM, 100 nM, 200 nM, 300 nM, 400 nM, 500 nM, 600 nM, 700 nM, 800 nM, 900 nM, 1 μM, 2 μM, 3 μM, 4 μM, 5 μM, 6 μM, 7 μM, 8 μM, 9 μM, 10 μM, 11 μM, 12 μM, 13 μM, 14 μM, 15 μM, 20 μM, 25 μM, 30 μM, 35 μM, 40 μM, 45 μM, or about 50 μM and the LY2090314 at a concentration of about 0.001 μM to 10 mM, about 0.01 μM to 1 mM, about 0.1 μM to 100 uM, about 0.001 μM to 0.01 μM, about 0.01 μM to 0.1 μM, about 0.1 μM to 1 μM, about 1 μM to 10 μM, about 10 μM to 100 μM, about 100 μM to 1 mM, or about 1 mM to 10 mM.

In some embodiments, the SGC0946 is in a concentration of about 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, 10 mM, 11 mM, 12 mM, 13 mM, 14 mM, 15 mM, 20 mM, 25 mM, 30 mM, 35 mM, 40 mM, 45 mM, or about 50 mM and the LY2090314 is at a concentration of about 1 μM, 5 μM, 10 μM, 15 μM, 20 μM, or 40 μM.

In some embodiments, the pharmaceutical composition comprises a DOT1L inhibitor that is SGC0946 and a GSK3 Inhibitor that is that is a substituted 3-Imidazo[1,2-a]pyridin-3-yl-4-(1,2,3,4-tetrahydro-[1,4]diazepino-[6,7,1-hi]indol-7-yl)pyrrole-2,5-dione. The SGC0946 is in a concentration of about 10 nM, 20 nM, 30 nM, 40 nM, 50 nM, 60 nM, 70 nM, 80 nM, 90 nM, 100 nM, 200 nM, 300 nM, 400 nM, 500 nM, 600 nM, 700 nM, 800 nM, 900 nM, 1 μM, 2 μM, 3 μM, 4 μM, 5 μM, 6 μM, 7 μM, 8 μM, 9 μM, 10 μM, 11 μM, 12 μM, 13 μM, 14 μM, 15 μM, 20 μM, 25 μM, 30 μM, 35 μM, 40 μM, 45 μM, or about 50 μM and the substituted 3-Imidazo[1,2-a]pyridin-3-yl-4-(1,2,3,4-tetrahydro-[1,4]diazepino-[6,7,1-hi]indol-7-yl)pyrrole-2,5-dione at a concentration of about 0.001 μM to 10 mM, about 0.01 μM to 1 mM, about 0.1 μM to 100 uM, about 0.001 μM to 0.01 μM, about 0.01 μM to 0.1 μM, about 0.1 μM to 1 μM, about 1 μM to 10 μM, about 10 μM to 100 μM, about 100 μM to 1 mM, or about 1 mM to 10 mM.

In some embodiments, the SGC0946 is in a concentration of about 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, 10 mM, 11 mM, 12 mM, 13 mM, 14 mM, 15 mM, 20 mM, 25 mM, 30 mM, 35 mM, 40 mM, 45 mM, or about 50 mM and the substituted 3-Imidazo[1,2-a]pyridin-3-yl-4-(1,2,3,4-tetrahydro-[1,4]diazepino-[6,7,1-hi]indol-7-yl)pyrrole-2,5-dione, is at a concentration of about 1 μM, 5 μM, 10 μM, 15 μM, 20 μM, 50 μM, 100 μM, 250 μM or 500 μM.

In some embodiments, the pharmaceutical composition comprises a DOT1L inhibitor that is SGC0946 and a GSK3 Inhibitor that is GSK3-inhibitor XXII. The SGC0946 is in a concentration of about 10 nM, 20 nM, 30 nM, 40 nM, 50 nM, 60 nM, 70 nM, 80 nM, 90 nM, 100 nM, 200 nM, 300 nM, 400 nM, 500 nM, 600 nM, 700 nM, 800 nM, 900 nM, 1 μM, 2 μM, 3 μM, 4 μM, 5 μM, 6 μM, 7 μM, 8 μM, 9 μM, 10 μM, 11 μM, 12 μM, 13 μM, 14 μM, 15 μM, 20 μM, 25 μM, 30 μM, 35 μM, 40 μM, 45 μM, or about 50 μM and the GSK3-inhibitor XXII, at a concentration of about 0.1 μM to 1,000 mM, about 1 μM to 100 mM, about 10 μM to 10 mM, about 0.1 μM to 1 μM, about 1 μM to 10 μM, about 10 μM to 100 μM, about 100 μM to 1 mM, about 1 mM to 10 mM, about 10 mM to 100 mM, or about 100 mM to 1000 mM.

In some embodiments, the SGC0946 is in a concentration of about 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, 10 mM, 11 mM, 12 mM, 13 mM, 14 mM, 15 mM, 20 mM, 25 mM, 30 mM, 35 mM, 40 mM, 45 mM, or about 50 mM and the GSK3-inhibitor XXII is at a concentration of about 0.1 mM, 0.2 mM, 0.3 mM, 0.4 mM, 0.5 mM, 0.6 mM, 0.7 mM, 0.8 mM, 0.9 mM, or 1.0 mM.

In some embodiments, the pharmaceutical composition comprises a DOT1L inhibitor that is SGC0946 and a GSK3 Inhibitor that is CHIR99021. The SGC0946 is in a concentration of about 10 nM, 20 nM, 30 nM, 40 nM, 50 nM, 60 nM, 70 nM, 80 nM, 90 nM, 100 nM, 200 nM, 300 nM, 400 nM, 500 nM, 600 nM, 700 nM, 800 nM, 900 nM, 1 μM, 2 μM, 3 μM, 4 μM, 5 μM, 6 μM, 7 μM, 8 μM, 9 μM, 10 μM, 11 μM, 12 μM, 13 μM, 14 μM, 15 μM, 20 μM, 25 μM, 30 μM, 35 μM, 40 μM, 45 μM, or about 50 μM and the CHIR99021 at a concentration of about 0.001 mM to 10,000 mM, about 0.01 mM to 1,000 mM, about 0.1 mM to 100 mM, about 0.001 mM to 0.01 mM, about 0.01 mM to 0.1 mM, about 0.1 mM to 1 mM, about 1 mM to 10 mM, about 10 mM to 100 mM, about 100 mM to 1,000 mM, or about 1,000 mM to 10,000 mM.

In some embodiments, the SGC0946 is in a concentration of about 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, 10 mM, 11 mM, 12 mM, 13 mM, 14 mM, 15 mM, 20 mM, 25 mM, 30 mM, 35 mM, 40 mM, 45 mM, or about 50 mM and the CHIR99021 is at a concentration of about 1 mM, 2 mM, 3 mM, 4 mM, 5 mM, 6 mM, 7 mM, 8 mM, 9 mM, or 10 mM.

In some embodiments, the pharmaceutical composition comprises a DOT1L inhibitor that is EPZ004777 and a GSK3 Inhibitor that is AZD1080 and a HDAC inhibitor that is VPA. The EPZ004777 is at a concentration of about 10 nM, 20 nM, 30 nM, 40 nM, 50 nM, 60 nM, 70 nM, 80 nM, 90 nM, 100 nM, 200 nM, 300 nM, 400 nM, 500 nM, 600 nM, 700 nM, 800 nM, 900 nM, 1 μM, 2 μM, 3 μM, 4 μM, 5 μM, 6 μM, 7 μM, 8 μM, 9 μM, 10 μM, 11 μM, 12 μM, 13 μM, 14 μM, 15 μM, 20 μM, 25 μM, 30 μM, 35 μM, 40 μM, 45 μM, or about 50 μM and the AZ1090 is at a concentration of about 0.001 mM to 10,000 mM, about 0.01 mM to 1,000 mM, about 0.1 mM to 100 mM, about 0.001 mM to 0.01 mM, about 0.01 mM to 0.1 mM, about 0.1 mM to 1 mM, about 1 mM to 10 mM, about 10 mM to 100 mM, about 100 mM to 1,000 mM, or about 1,000 mM to 10,000 mM and the VPA at a concentration about 100 mM to 4,000 mM.

In some embodiments, the EPZ004777 at a concentration of about 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, 10 mM, 11 mM, 12 mM, 13 mM, 14 mM, 15 mM, 20 mM, 25 mM, 30 mM, 35 mM, 40 mM, 45 mM, or about 50 mM and the AZ1090 is at a a concentration of about 1 mM, 2 mM, 3 mM, 4 mM, 5 mM, 6 mM, 7 mM, 8 mM, 9 mM, or 10 mM and the VPA at a concentration about 100 mM to 4,000 mM.

In some embodiments, the pharmaceutical composition comprises a DOT1L inhibitor that is EPZ004777 and a GSK3 Inhibitor that is LY2090314 and a HDAC inhibitor that is VPA. The EPZ004777 is at a concentration of about 10 nM, 20 nM, 30 nM, 40 nM, 50 nM, 60 nM, 70 nM, 80 nM, 90 nM, 100 nM, 200 nM, 300 nM, 400 nM, 500 nM, 600 nM, 700 nM, 800 nM, 900 nM, 1 μM, 2 μM, 3 μM, 4 μM, 5 μM, 6 μM, 7 μM, 8 μM, 9 μM, 10 μM, 11 μM, 12 μM, 13 μM, 14 μM, 15 μM, 20 μM, 25 μM, 30 μM, 35 μM, 40 μM, 45 μM, or about 50 μM and the LY2090314 at a concentration of about 0.001 μM to 10 mM, about 0.01 μM to 1 mM, about 0.1 μM to 100 uM, about 0.001 μM to 0.01 μM, about 0.01 μM to 0.1 μM, about 0.1 μM to 1 μM, about 1 μM to 10 μM, about 10 μM to 100 μM, about 100 μM to 1 mM, or about 1 mM to 10 mM and the VPA at a concentration about 100 mM to 4,000 mM.

In some embodiments, the EPZ004777 at a concentration of about 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, 10 mM, 11 mM, 12 mM, 13 mM, 14 mM, 15 mM, 20 mM, 25 mM, 30 mM, 35 mM, 40 mM, 45 mM, or about 50 mM and the LY2090314 is at a concentration of about 1 μM, 5 μM, 10 μM, 15 μM, 20 μM or 40 μM and the VPA at a concentration about 100 mM to 4,000 mM.

In some embodiments, the pharmaceutical composition comprises a DOT1L inhibitor that is EPZ004777 and a GSK3 Inhibitor that is that is a substituted 3-Imidazo[1,2-a]pyridin-3-yl-4-(1,2,3,4-tetrahydro-[1,4]diazepino-[6,7,1-hi]indol-7-yl)pyrrole-2,5-dione and a HDAC inhibitor that is VPA. The EPZ004777 is at a concentration of about 10 nM, 20 nM, 30 nM, 40 nM, 50 nM, 60 nM, 70 nM, 80 nM, 90 nM, 100 nM, 200 nM, 300 nM, 400 nM, 500 nM, 600 nM, 700 nM, 800 nM, 900 nM, 1 μM, 2 μM, 3 μM, 4 μM, 5 μM, 6 μM, 7 μM, 8 μM, 9 μM, 10 μM, 11 μM, 12 μM, 13 μM, 14 μM, 15 μM, 20 μM, 25 μM, 30 μM, 35 μM, 40 μM, 45 μM, or about 50 μM and the substituted 3-Imidazo[1,2-a]pyridin-3-yl-4-(1,2,3,4-tetrahydro-[1,4]diazepino-[6,7,1-hi]indol-7-yl)pyrrole-2,5-dione at a concentration of about 0.001 μM to 10 mM, about 0.01 μM to 1 mM, about 0.1 μM to 100 uM, about 0.001 μM to 0.01 μM, about 0.01 μM to 0.1 μM, about 0.1 μM to 1 μM, about 1 μM to 10 μM, about 10 μM to 100 μM, about 100 μM to 1 mM, or about 1 mM to 10 mM and the VPA at a concentration about 100 mM to 4,000 mM.

In some embodiments, the EPZ004777 at a concentration of about 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, 10 mM, 11 mM, 12 mM, 13 mM, 14 mM, 15 mM, 20 mM, 25 mM, 30 mM, 35 mM, 40 mM, 45 mM, or about 50 mM and the substituted 3-Imidazo[1,2-a]pyridin-3-yl-4-(1,2,3,4-tetrahydro-[1,4]diazepino-[6,7,1-hi]indol-7-yl)pyrrole-2,5-dione, is at a concentration of about 1 μM, 5 μM, 10 μM, 15 μM, 20 μM, 50 μM, 100 μM, 250 μM or 500 μM and the VPA at a concentration about 100 mM to 4,000 mM.

In some embodiments, the pharmaceutical composition comprises a DOT1L inhibitor that is EPZ004777 and a GSK3 Inhibitor that is GSK3-inhibitor XXII and a HDAC inhibitor that is VPA. The EPZ004777 is at a concentration of about 10 nM, 20 nM, 30 nM, 40 nM, 50 nM, 60 nM, 70 nM, 80 nM, 90 nM, 100 nM, 200 nM, 300 nM, 400 nM, 500 nM, 600 nM, 700 nM, 800 nM, 900 nM, 1 μM, 2 μM, 3 μM, 4 μM, 5 μM, 6 μM, 7 μM, 8 μM, 9 μM, 10 μM, 11 μM, 12 μM, 13 μM, 14 μM, 15 μM, 20 μM, 25 μM, 30 μM, 35 μM, 40 μM, 45 μM, or about 50 μM and the GSK3-inhibitor XXII, at a concentration of about 0.1 μM to 1,000 mM, about 1 μM to 100 mM, about 10 μM to 10 mM, about 0.1 μM to 1 μM, about 1 μM to 10 μM, about 10 μM to 100 μM, about 100 μM to 1 mM, about 1 mM to 10 mM, about 10 mM to 100 mM, or about 100 mM to 1000 mM and the VPA at a concentration about 100 mM to 4,000 mM.

In some embodiments, the EPZ004777 at a concentration of about 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, 10 mM, 11 mM, 12 mM, 13 mM, 14 mM, 15 mM, 20 mM, 25 mM, 30 mM, 35 mM, 40 mM, 45 mM, or about 50 mM and the GSK3-inhibitor XXII is at a concentration of about 0.1 mM, 0.2 mM, 0.3 mM, 0.4 mM, 0.5 mM, 0.6 mM, 0.7 mM, 0.8 mM, 0.9 mM, or 1.0 mM and the VPA at a concentration about 100 mM to 4,000 mM.

In some embodiments, the pharmaceutical composition comprises a DOT1L inhibitor that is EPZ004777 and a GSK3 Inhibitor that is CHIR99021 and a HDAC inhibitor that is VPA. The EPZ004777 is at a concentration of about 10 nM, 20 nM, 30 nM, 40 nM, 50 nM, 60 nM, 70 nM, 80 nM, 90 nM, 100 nM, 200 nM, 300 nM, 400 nM, 500 nM, 600 nM, 700 nM, 800 nM, 900 nM, 1 μM, 2 μM, 3 μM, 4 μM, 5 μM, 6 μM, 7 μM, 8 μM, 9 μM, 10 μM, 11 μM, 12 μM, 13 μM, 14 μM, 15 μM, 20 μM, 25 μM, 30 μM, 35 μM, 40 μM, 45 μM, or about 50 μM and the CHIR99021 at a concentration of about 0.001 mM to 10,000 mM, about 0.01 mM to 1,000 mM, about 0.1 mM to 100 mM, about 0.001 mM to 0.01 mM, about 0.01 mM to 0.1 mM, about 0.1 mM to 1 mM, about 1 mM to 10 mM, about 10 mM to 100 mM, about 100 mM to 1,000 mM, or about 1,000 mM to 10,000 mM and the VPA at a concentration about 100 mM to 4,000 mM.

In some embodiments, the EPZ004777 at a concentration of about 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, 10 mM, 11 mM, 12 mM, 13 mM, 14 mM, 15 mM, 20 mM, 25 mM, 30 mM, 35 mM, 40 mM, 45 mM, or about 50 mM and the CHIR99021 is at a concentration of about 1 mM, 2 mM, 3 mM, 4 mM, 5 mM, 6 mM, 7 mM, 8 mM, 9 mM, or 10 mM and the VPA at a concentration about 100 mM to 4,000 mM.

In some embodiments, the pharmaceutical composition comprises a KDM inhibitor that is TC-E 5002 and a GSK3 Inhibitor that is AZD1080. The TC-E 5002 is at a concentration of about 0.01 μM to 1000 mM, about 0.1 μM to 100 mM, about 1 μM to 10 mM, about 10 μM to 1000 μM, about 1 μM to 10 μM, 10 μM to 100 μM, about 100 μM to 1000 μM or about 1 mM to 10 mM and the AZ1090 is at a concentration of about 0.001 mM to 10,000 mM, about 0.01 mM to 1,000 mM, about 0.1 mM to 100 mM, about 0.001 mM to 0.01 mM, about 0.01 mM to 0.1 mM, about 0.1 mM to 1 mM, about 1 mM to 10 mM, about 10 mM to 100 mM, about 100 mM to 1,000 mM, or about 1,000 mM to 10,000 mM.

In some embodiments, the TC-E 5002 at a concentration of about 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, or 10 mM and the AZ1090 is at a a concentration of about 1 mM, 2 mM, 3 mM, 4 mM, 5 mM, 6 mM, 7 mM, 8 mM, 9 mM, or 10 mM.

In some embodiments, the pharmaceutical composition comprises a KDM inhibitor that is TC-E 5002 and a GSK3 Inhibitor that is LY2090314. The TC-E 5002 is at a concentration of about 0.01 μM to 1000 mM, about 0.1 μM to 100 mM, about 1 μM to 10 mM, about 10 μM to 1000 μM, about 1 μM to 10 μM, 10 μM to 100 μM, about 100 μM to 1000 μM or about 1 mM to 10 mM and the LY2090314 at a concentration of about 0.001 μM to 10 mM, about 0.01 μM to 1 mM, about 0.1 μM to 100 uM, about 0.001 μM to 0.01 μM, about 0.01 μM to 0.1 μM, about 0.1 μM to 1 μM, about 1 μM to 10 μM, about 10 μM to 100 μM, about 100 μM to 1 mM, or about 1 mM to 10 mM.

In some embodiments, the TC-E 5002 at a concentration of about 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, or 10 mM and the LY2090314 the is at a concentration of about 1 μM, 5 μM, 10 μM, 15 μM, 20 μM or 40 μM.

In some embodiments, the pharmaceutical composition comprises a KDM inhibitor that is TC-E 5002 and a GSK3 inhibitor that is that is a substituted 3-Imidazo[1,2-a]pyridin-3-yl-4-(1,2,3,4-tetrahydro-[1,4]diazepino-[6,7,1-hi]indol-7-yl)pyrrole-2,5-dione. The TC-E 5002 is at a concentration of about 0.01 μM to 1000 mM, about 0.1 μM to 100 mM, about 1 μM to 10 mM, about 10μM to 1000 μM, about 1 μM to 10 μM, 10 μM to 100 μM, about 100 μM to 1000 μM or about 1 mM to 10 mM and the substituted 3-Imidazo[1,2-a]pyridin-3-yl-4-(1,2,3,4-tetrahydro-[1,4]diazepino-[6,7,1-hi]indol-7-yl)pyrrole-2,5-dione at a concentration of about 0.001 μM to 10 mM, about 0.01 μM to 1 mM, about 0.1 μM to 100 uM, about 0.001 μM to 0.01 μM, about 0.01 μM to 0.1 μM, about 0.1 μM to 1 μM, about 1 μM to 10 μM, about 10 μM to 100 μM, about 100 μM to 1 mM, or about 1 mM to 10 mM.

In some embodiments, the TC-E 5002 at a concentration of about 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, or 10 mM and the substituted 3-Imidazo[1,2-a]pyridin-3-yl-4-(1,2,3,4-tetrahydro-[1,4]diazepino-[6,7,1-hi]indol-7-yl)pyrrole-2,5-dione, is at a concentration of about 1 μM, 5 μM, 10 μM, 15 μM, 20 μM, 50 μM, 100 μM, 250 μM or 500 μM.

In some embodiments, the pharmaceutical composition comprises a KDM inhibitor that is TC-E 5002 and a GSK3 Inhibitor that is GSK3-inhibitor XXII. The TC-E 5002 is at a concentration of about 0.01 μM to 1000 mM, about 0.1 μM to 100 mM, about 1 μM to 10 mM, about 10 μM to 1000 μM, about 1 μM to 10 μM, 10 μM to 100 μM, about 100 μM to 1000 μM or about 1 mM to 10 mM and the GSK3-inhibitor XXII, at a concentration of about of about 0.1 μM to 1,000 mM, about 1 μM to 100 mM, about 10 μM to 10 mM, about 0.1 μM to 1 μM, about 1 μM to 10 μM, about 10 μM to 100 μM, about 100 μM to 1 mM, about 1 mM to 10 mM, about 10 mM to 100 mM, or about 100 mM to 1000 mM.

In some embodiments, the TC-E 5002 at a concentration of about 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, or 10 mM and the GSK3-inhibitor XXII is at a concentration of about 0.1 mM, 0.2 mM, 0.3 mM, 0.4 mM, 0.5 mM, 0.6 mM, 0.7 mM, 0.8 mM, 0.9 mM, or 1.0 mM.

In some embodiments, the pharmaceutical composition comprises a KDM inhibitor that is TC-E 5002 and a GSK3 Inhibitor that is CHIR99021. The TC-E 5002 is at a concentration of about 0.01 μM to 1000 mM, about 0.1 μM to 100 mM, about 1 μM to 10 mM, about 10 μM to 1000 μM, about 1 μM to 10 μM, 10 μM to 100 μM, about 100 μM to 1000 μM or about 1 mM to 10 mM and the CHIR99021 at a concentration of about 0.001 mM to 10,000 mM, about 0.01 mM to 1,000 mM, about 0.1 mM to 100 mM, about 0.001 mM to 0.01 mM, about 0.01 mM to 0.1 mM, about 0.1 mM to 1 mM, about 1 mM to 10 mM, about 10 mM to 100 mM, about 100 mM to 1,000 mM, or about 1,000 mM to 10,000 mM.

In some embodiments, the TC-E 5002 at a concentration of about 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, or 10 mM and the CHIR99021 is at a concentration of about 1 mM, 2 mM, 3 mM, 4 mM, 5 mM, 6 mM, 7 mM, 8 mM, 9 mM, or 10 mM.

In some embodiments, the pharmaceutical composition comprises a KDM inhibitor that is AS 8351 and a GSK3 Inhibitor that is AZD1080. The AS 8351 is at a concentration of about 0.01 μM to 1000 mM, about 0.1 μM to 100 mM, about 1 μM to 10 mM, about 10 μM to 1000 μM, about 1 μM to 10 μM, 10 μM to 100 μM, about 100 μM to 1000 μM or about 1 mM to 10 mM and the AZ1090 is at a concentration of about 0.001 mM to 10,000 mM, about 0.01 mM to 1,000 mM, about 0.1 mM to 100 mM, about 0.001 mM to 0.01 mM, about 0.01 mM to 0.1 mM, about 0.1 mM to 1 mM, about 1 mM to 10 mM, about 10 mM to 100 mM, about 100 mM to 1,000 mM, or about 1,000 mM to 10,000 mM.

In some embodiments, the AS 8351 at a concentration of about 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, or 10 mM and the AZ1090 is at a a concentration of about 1 mM, 2 mM, 3 mM, 4 mM, 5 mM, 6 mM, 7 mM, 8 mM, 9 mM, or 10 mM.

In some embodiments, the pharmaceutical composition comprises a KDM inhibitor that is AS 8351 and a GSK3 Inhibitor that is LY2090314. The AS 8351 is at a concentration of about 0.01 μM to 1000 mM, about 0.1 μM to 100 mM, about 1 μM to 10 mM, about 10 μM to 1000 μM, about 1 μM to 10 μM, 10 μM to 100 μM, about 100 μM to 1000 μM or about 1 mM to 10 mM and the LY2090314 at a concentration of about 0.001 μM to 10 mM, about 0.01 μM to 1 mM, about 0.1 μM to 100 uM, about 0.001 μM to 0.01 μM, about 0.01 μM to 0.1 μM, about 0.1 μM to 1 μM, about 1 μM, to 10 μM, about 10 μM, to 100 μM, about 100 μM to 1 mM, or about 1 mM to 10 mM.

In some embodiments, the AS 8351 at a concentration of about 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, or 10 mM and the LY2090314 the is at a concentration of about 1 μM, 5 μM, 10 μM, 15 μM, 20 μM or 40 μM.

In some embodiments, the pharmaceutical composition comprises a KDM inhibitor that is AS 8351 and a GSK3 Inhibitor that is that is a substituted 3-Imidazo[1,2-a]pyridin-3-yl-4-(1,2,3,4-tetrahydro-[1,4]diazepino-[6,7,1-hi]indol-7-yl)pyrrole-2,5-dione. The AS 8351 is at a concentration of about 0.01 μM to 1000 mM, about 0.1 μM to 100 mM about 1 μM to 10 mM, about 10 μM to 1000 μM, about 1 μM to 10 μM, 10 μM to 100 μM, about 100 μM to 1000 μM or about 1 mM to 10 mM and the substituted 3-Imidazo[1,2-a]pyridin-3-yl-4-(1,2,3,4-tetrahydro-[1,4]diazepino-[6,7,1-hi]indol-7-yl)pyrrole-2,5-dione at a concentration of about 0.001 μM to 10 mM, about 0.01 μM to 1 mM, about 0.1 μM to 100 uM, about 0.001 μM to 0.01 μM, about 0.01 μM to 0.1 μM, about 0.1 μM to 1 μM, about 1 μM to 10 μM, about 10 μM to 100 μM, about 100 μM to 1 mM, or about 1 mM to 10 mM.

In some embodiments, the AS 8351 at a concentration of about 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, or 10 mM and the substituted 3-Imidazo[1,2-a]pyridin-3-yl-4-(1,2,3,4-tetrahydro-[1,4]diazepino-[6,7,1-hi]indol-7-yl)pyrrole-2,5-dione, is at a concentration of about 1 μM, 5 μM, 10 μM, 15 μM, 20 μM, 50 μM, 100 μM, 250 μM or 500 μM.

In some embodiments, the pharmaceutical composition comprises a KDM inhibitor that is AS 8351 and a GSK3 Inhibitor that is GSK3-inhibitor XXII. The AS 8351 is at a concentration of about 0.01 μM to 1000 mM, about 0.1 μM to 100 mM, about 1 μM to 10 mM, about 10 μM to 1000 μM, about 1 μM to 10 μM, 10 μM to 100 μM, about 100 μM to 1000 μM or about 1 mM to 10 mM and the GSK3-inhibitor XXII, at a concentration of about of about 0.1 μM to 1,000 mM, about 1 μM to 100 mM, about 10 μM to 10 mM, about 0.1 μM to 1 μM, about 1 μM to 10 μM, about 10 μM to 100 μM, about 100 μM to 1 mM, about 1 mM to 10 mM, about 10 mM to 100 mM, or about 100 mM to 1000 mM.

In some embodiments, the AS 8351 at a concentration of about 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, or 10 mM and the GSK3-inhibitor XXII is at a concentration of about 0.1 mM, 0.2 mM, 0.3 mM, 0.4 mM, 0.5 mM, 0.6 mM, 0.7 mM, 0.8 mM, 0.9 mM, or 1.0 mM.

In some embodiments, the pharmaceutical composition comprises a KDM inhibitor that is AS 8351 and a GSK3 Inhibitor that is CHIR99021. The AS 8351 is at a concentration of about 0.01 μM to 1000 mM, about 0.1 μM to 100 mM, about 1 μM to 10 mM, about 10 μM to 1000 μM, about 1 μM to 10 μM, 10 μM to 100 μM, about 100 μM to 1000 μM or about 1 mM to 10 mM and the CHIR99021 at a concentration of about 0.001 mM to 10,000 mM, about 0.01 mM to 1,000 mM, about 0.1 mM to 100 mM, about 0.001 mM to 0.01 mM, about 0.01 mM to 0.1 mM, about 0.1 mM to 1 mM, about 1 mM to 10 mM, about 10 mM to 100 mM, about 100 mM to 1,000 mM, or about 1,000 mM to 10,000 mM.

In some embodiments, the AS 8351 at a concentration of about 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, or 10 mM and the CHIR99021 is at a concentration of about 1 mM, 2 mM, 3 mM, 4 mM, 5 mM, 6 mM, 7 mM, 8 mM, 9 mM, or 10 mM.

In some embodiments, the pharmaceutical composition comprises a KDM inhibitor that is EPT-103182 and a GSK3 Inhibitor that is AZD1080. The EPT-103182 is at a concentration of 0.001 μM to 100 mM, about 0.01 μM to 10 mM, about 0.1 μM to 1 mM, about 1 μM to 100 μM, about 1 μM to 10 μM, 10 μM to 100 μM, or about 100 μM to 1 mM and the AZ1090 is at a concentration of about 0.001 mM to 10,000 mM, about 0.01 mM to 1,000 mM, about 0.1 mM to 100 mM, about 0.001 mM to 0.01 mM about 0.01 mM to 0.1 mM, about 0.1 mM to 1 mM, about 1 mM to 10 mM, about 10 mM to 100 mM, about 100 mM to 1,000 mM, or about 1,000 mM to 10,000 mM.

In some embodiments, the EPT-103182 at a concentration of about 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, or about 1 mM and the AZ1090 is at a a concentration of about 1 mM, 2 mM, 3 mM, 4 mM, 5 mM, 6 mM, 7 mM, 8 mM, 9 mM, or 10 mM.

In some embodiments, the pharmaceutical composition comprises a KDM inhibitor that is EPT-103182 and a GSK3 Inhibitor that is LY2090314. The EPT-103182 is at a concentration of 0.001 μM to 100 mM, about 0.01 μM to 10 mM, about 0.1 μM to 1 mM, about 1 μM to 100 μM, about 1 μM to 10 μM, 10 μM to 100 μM, or about 100 μM to 1 mM and the LY2090314 at a concentration of about 0.001 μM to 10 mM, about 0.01 μM to 1 mM, about 0.1 μM to 100 uM, about 0.001 μM to 0.01 μM, about 0.01 μM to 0.1 μM, about 0.1 μM to 1 μM, about 1 μM to 10 μM, about 10 μM to 100 μM, about 100 μM to 1 mM, or about 1 mM to 10 mM.

In some embodiments, the EPT-103182 at a concentration of about 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, or about 1 mM and the LY2090314 the is at a concentration of about 1 μM, 5 μM, 10 μM, 15 μM, 20 μM or 40 μM.

In some embodiments, the pharmaceutical composition comprises a KDM inhibitor that is EPT-103182 and a GSK3 Inhibitor that is that is a substituted 3-Imidazo[1,2-a]pyridin-3-yl-4-(1,2,3,4-tetrahydro-[1,4]diazepino-[6,7,1-hi]indol-7-yl)pyrrole-2,5-dione. The EPT-103182 is at a concentration of 0.001 μM to 100 mM, about 0.01 μM to 10 mM, about 0.1 μM to 1 mM, about 1 μM to 100 μM, about 1 μM to 10 μM, 10 μM to 100 μM, or about 100 μM to 1 mM and the substituted 3-Imidazo[1,2-a]pyridin-3-yl-4-(1,2,3,4-tetrahydro-[1,4]diazepino-[6,7,1-hi]indol-7-yl)pyrrole-2,5-dione at a concentration of about 0.001 μM to 10 mM, about 0.01 μM to 1 mM, about 0.1 μM to 100 uM, about 0.001 μM to 0.01 μM, about 0.01 μM to 0.1 μM, about 0.1 μM to 1 μM, about 1 μM to 10 μM, about 10 μM to 100 μM, about 100 μM to 1 mM, or about 1 mM to 10 mM.

In some embodiments, the EPT-103182 at a concentration of about 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, or about 1 mM and the substituted 3-Imidazo[1,2-a]pyridin-3-yl-4-(1,2,3,4-tetrahydro-[1,4]diazepino-[6,7,1-hi]indol-7-yl)pyrrole-2,5-dione, is at a concentration of about 1 μM, 5 μM, 10 μM, 15 μM, 20 μM, 50 μM, 100 μM, 250 μM, or 500 μM.

In some embodiments, the pharmaceutical composition comprises a KDM inhibitor that is EPT-103182 and a GSK3 Inhibitor that is GSK3-inhibitor XXII. The EPT-103182 is at a concentration of 0.001 μM to 100 mM, about 0.01 μM to 10 mM, about 0.1 μM to 1 mM, about 1 μM to 100 μM, about 1 μM to 10 μM, 10 μM to 100 μM, or about 100 μM to 1 mM and the GSK3-inhibitor XXII, at a concentration of about of about 0.1 μM to 1,000 mM, about 1 μM to 100 mM, about 10 μM to 10 mM, about 0.1 μM to 1 μM, about 1 μM to 10 μM, about 10 μM to 100 μM, about 100 μM to 1 mM about 1 mM to 10 mM, about 10 mM to 100 mM, or about 100 mM to 1000 mM.

In some embodiments, the EPT-103182 at a concentration of about 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, or about 1 mM and the GSK3-inhibitor XXII is at a concentration of about 0.1 mM, 0.2 mM, 0.3 mM, 0.4 mM, 0.5 mM, 0.6 mM, 0.7 mM, 0.8 mM, 0.9 mM, or 1.0 mM

In some embodiments, the pharmaceutical composition comprises a KDM inhibitor that is EPT-103182 and a GSK3 Inhibitor that is CHIR99021. The EPT-103182 is at a concentration of 0.001 μM to 100 mM, about 0.01 μM to 10 mM, about 0.1. μM to 1 mM, about 1 μM to 100 μM, about 1 μM to 10 μM, 10 μM to 100 μM, or about 100 μM to 1 mM and the CHIR99021 at a concentration of about 0.001 mM to 10,000 mM, about 0.01 mM to 1,000 mM, about 0.1 mM to 100 mM, about 0.001 mM to 0.01 mM, about 0.01 mM to 0.1 mM, about 0.1 mM to 1 mM, about 1 mM to 10 mM, about 10 mM to 100 mM, about 100 mM to 1,000 mM, or about 1,000 mM to 10,000 mM.

In some embodiments, the EPT-103182 at a concentration of about 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, or about 1 mM and the CHIR99021 is at a concentration of about 1 mM, 2 mM, 3 mM, 4 mM, 5 mM, 6 mM, 7 mM, 8 mM, 9 mM, or 10 mM.

In some embodiments, the pharmaceutical composition comprises a KDM inhibitor that is TC-E 5002 and a GSK3 Inhibitor that is AZD1080 and a HDAC inhibitor that is VPA. The TC-E 5002 is at a concentration of about 0.01 μM to 1000 mM, about 0.1 μM to 100 mM, about 1 μM to 10 mM, about 10 μM to 1000 μM, about 1 μM to 10 μM, 10 μM to 100 μM, about 100 μM to 1000 μM or about 1 mM to 10 mM and the AZ1090 is at a concentration of about 0.001 mM to 10,000 mM, about 0.01 mM to 1,000 mM, about 0.1 mM to 100 mM, about 0.001 mM to 0.01 mM, about 0.01 mM to 0.1 mM, about 0.1 mM to 1 mM, about 1 mM to 10 mM, about 10 mM to 100 mM, about 100 mM to 1,000 mM, or about 1,000 mM to 10,000 mM and the VPA at a concentration about 100 mM to 4,000 mM.

In some embodiments, the TC-E 5002 at a concentration of about 1.0 μM, 2.0 μM, 3.0 μM, 4.0 μM, 5.0 M, 6.0 μM, 7.0 M, 8.0 M, 9.0 μM, 10 μM, 20 μM, 30 μM, 40 μM, 50 μM, 60 μM, 70 μM, 80 μM, 90 μM, 100 μM, 200 μM, 300 μM, 400 M, 500 μM, 600 μM, 700 μM, 800 μM, 900 μM, 1 mM, 2 mM, 3 mM, 4 mM, 5 mM, 6 mM, 7 mM, 8 mM, 9 mM, or 10 mM and the AZ1090 is at a a concentration of about 1 mM, 2 mM, 3 mM, 4 mM, 5 mM, 6 mM, 7 mM, 8 mM, 9 mM, or 10 mM and the VPA at a concentration about 100 mM to 4,000 mM.

In some embodiments, the pharmaceutical composition comprises a KDM inhibitor that is TC-E 5002 and a GSK3 Inhibitor that is LY2090314 and a HDAC inhibitor that is VPA. The TC-E 5002 is at a concentration of about 0.01 μM to 1000 mM, about 0.1 μM to 100 mM, about 1 μM to 10 mM about 10 μM to 1000 μM, about 1 μM to 10 μM, 10 μM to 100 μM, about 100 μM to 1000 μM or about 1 mM to 10 mM and the LY2090314 at a concentration of about 0.001 μM to 10 mM, about 0.01 μM to 1 mM, about 0.1 μM to 100 uM, about 0.001 μM to 0.01 μM, about 0.01 μM to 0.1 μM, about 0.1 μM to 1 μM, about 1 μM to 10 μM, about 10 μM to 100 μM, about 100 μM to 1 mM, or about 1 mM to 10 mM and the VPA at a concentration about 100 mM to 4,000 mM.

In some embodiments, the TC-E 5002 at a concentration of about 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, or 10 mM and the LY2090314 the is at a concentration of about 1 μM, 5 μM, 10 μM, 15 μM, 20 μM or 40 μM and the VPA at a concentration about 100 mM to 4,000 mM.

In some embodiments, the pharmaceutical composition comprises a KDM inhibitor that is TC-E 5002 and a GSK3 Inhibitor that is that is a substituted 3-Imidazo[1,2-a]pyridin-3-yl-4-(1,2,3,4-tetrahydro-[1,4]diazepino-[6,7,1-hi]indol-7-yl)pyrrole-2,5-dione and a HDAC inhibitor that is VPA. The TC-E 5002 is at a concentration of about 0.01 μM to 1000 mM, about 0.1 μM to 100 mM, about 1 μM to 10 mM, about 10 μM to 1000 μM, about 1 μM to 10 μM, 10 μM to 100 μM, about 100 μM to 1000 μM or about 1 mM to 10 mM and the substituted 3-Imidazo[1,2-a]pyridin-3-yl-4-(1,2,3,4-tetrahydro-[1,4]diazepino-[6,7,1-hi]indol-7-yl)pyrrole-2,5-dione at a concentration of about 0.001 μM to 10 mM, about 0.01 μM to 1 mM, about 0.1 μM to 100 uM, about 0.001 μM to 0.01 μM, about 0.01 μM to 0.1 μM, about 0.1 μM to 1 μM, about 1 μM to 10 μM, about 10 μM to 100 μM, about 100 μM to 1 mM, or about 1 mM to 10 mM. and the VPA at a concentration about 100 mM to 4,000 mM.

In some embodiments, the TC-E 5002 at a concentration of about 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, or 10 mM and the substituted 3-Imidazo[1,2-a]pyridin-3-yl-4-(1,2,3,4-tetrahydro-[1,4]diazepino-[6,7,1-hi]indol-7-yl)pyrrole-2,5-dione, is at a concentration of about 1 μM, 5 μM, 10 μM, 15 μM, 20 μM, 50 μM, 100 μM, 250 μM or 500 μM and the VPA at a concentration about 100 mM to 4,000 mM.

In some embodiments, the pharmaceutical composition comprises a KDM inhibitor that is TC-E 5002 and a GSK3 Inhibitor that is GSK3-inhibitor XXII and a HDAC inhibitor that is VPA. The TC-E 5002 is at a concentration of about 0.01 μM to 1000 mM, about 0.1 μM to 100 mM, about 1 μM to 10 mM, about 10 μM to 1000 μM, about 1 μM to 10 μM, 10 μM to 100 μM, about 100 μM to 1000 μM or about 1 mM to 10 mM and the GSK3-inhibitor XXII, at a concentration of about of about 0.1 μM to 1,000 mM, about 1 μM to 100 mM, about 10 μM to 10 mM, about 0.1 μM to 1 μM, about 1 μM to 10 μM, about 10 μM to 100 μM, about 100 μM to 1 mM, about 1 mM to 10 mM, about 10 mM to 100 mM, or about 100 mM to 1000 mM and the VPA at a concentration about 100 mM to 4,000 mM.

In some embodiments, the TC-E 5002 at a concentration of about 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, or 10 mM and the GSK3-inhibitor XXII is at a concentration of about 0.1 mM, 0.2 mM, 0.3 mM, 0.4 mM, 0.5 mM, 0.6 mM, 0.7 mM, 0.8 mM, 0.9 mM, or 1.0 mM. and the VPA at a concentration about 100 mM to 4,000 mM.

In some embodiments, the pharmaceutical composition comprises a KDM inhibitor that is TC-E 5002 and a GSK3 Inhibitor that is CHIR99021 and a HDAC inhibitor that is VPA. The TC-E 5002 is at a concentration of about 0.01 μM to 1000 mM, about 0.1 μM to 100 mM, about 1 μM to 10 mM, about 10 μM to 1000 μM, about 1 μM to 10 μM, 10 μM to 100 μM, about 100 μM to 1000 μM or about 1 mM to 10 mM and the CHIR99021 at a concentration of about 0.001 mM to 10,000 mM, about 0.01 mM to 1,000 mM, about 0.1 mM to 100 mM, about 0.001 mM to 0.01 mM, about 0.01 mM to 0.1 mM, about 0.1 mM to 1 mM, about 1 mM to 10 mM, about 10 mM to 100 mM, about 100 mM to 1,000 mM, or about 1,000 mM to 10,000 mM and the VPA at a concentration about 100 mM to 4,000 mM.

In some embodiments, the TC-E 5002 at a concentration of about 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, or 10 mM and the CHIR99021 is at a concentration of about 1 mM, 2 mM, 3 mM, 4 mM, 5 mM, 6 mM, 7 mM, 8 mM, 9 mM, or 10 mM and the VPA at a concentration about 100 mM to 4,000 mM.

In some embodiments, the pharmaceutical composition comprises a KDM inhibitor that is AS 8351 and a GSK3 Inhibitor that is AZD1080 and a HDAC inhibitor that is VPA. The AS 8351 is at a concentration of about 0.01 μM to 1000 mM, about 0.1 μM to 100 mM, about 1 μM to 10 mM, about 10 μM to 1000 μM, about 1 μM to 10 μM, 10 μM to 100 μM, about 100 μM to 1000 μM or about 1 mM to 10 mM and the AZ1090 is at a concentration of about 0.001 mM to 10,000 mM, about 0.01 mM to 1,000 mM, about 0.1 mM to 100 mM, about 0.001 mM to 0.01 mM, about 0.01 mM to 0.1 mM, about 0.1 mM to 1 mM, about 1 mM to 10 mM, about 10 mM to 100 mM, about 100 mM to 1,000 mM, or about 1,000 mM to 10,000 mM and the VPA at a concentration about 100 mM to 4,000 mM.

In some embodiments, the AS 8351 at a concentration of about 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, or 10 mM and the AZ1090 is at a a concentration of about 1 mM, 2 mM, 3 mM, 4 mM, 5 mM, 6 mM, 7 mM, 8 mM, 9 mM, or 10 mM and the VPA at a concentration about 100 mM to 4,000 mM.

In some embodiments, the pharmaceutical composition comprises a KDM inhibitor that is AS 8351 and a GSK3 Inhibitor that is LY2090314 and a HDAC inhibitor that is VPA. The AS 8351 is at a concentration of about 0.01 μM to 1000 mM, about 0.1 μM to 100 mM, about 1 μM to 10 mM, about 10 μM to 1000 μM, about 1 μM to 10 μM, 10 μM to 100 μM, about 100 μM to 1000 μM or about 1 mM to 10 mM and the LY2090314 at a concentration of about 0.001 μM to 10 mM, about 0.01 μM to 1 mM, about 0.1 μM to 100 uM, about 0.001 μM to 0.01 μM, about 0.01 μM to 0.1 μM, about 0.1 μM to 1 μM, about 1 μM to 10 μM, about 10 μM to 100 μM, about 100 μM to 1 mM, or about 1 mM to 10 mM and the VPA at a concentration about 100 mM to 4,000 mM.

In some embodiments, the AS 8351 at a concentration of about 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, or 10 mM and the LY2090314 the is at a concentration of about 1 μM, 5 μM, 10 μM, 15 μM, 20 μM or 40 μM and the VPA at a concentration about 100 mM to 4,000 mM.

In some embodiments, the pharmaceutical composition comprises a KDM inhibitor that is AS 8351 and a GSK3 Inhibitor that is that is a substituted 3-Imidazo[1,2-a]pyridin-3-yl-4-(1,2,3,4-tetrahydro-[1,4]diazepino-[6,7,1-hi]indol-7-yl)pyrrole-2,5-dione and a HDAC inhibitor that is VPA. The AS 8351 is at a concentration of about 0.01 μM to 1000 mM, about 0.1 μM to 100 mM, about 1 μM to 10 mM, about 10 μM to 1000 μM, about 1 μM to 10 μM, 10 μM to 100 μM, about 100 μM to 1000 μM or about 1 mM to 10 mM and the substituted 3-Imidazo[1,2-a]pyridin-3-yl-4-(1,2,3,4-tetrahydro-[1,4]diazepino-[6,7,1-hi]indol-7-yl)pyrrole-2,5-dione at a concentration of about 0.001 μM to 10 mM, about 0.01 μM to 1 mM, about 0.1 μM to 100 uM, about 0.001 μM to 0.01 μM, about 0.01 μM to 0.1 μM, about 0.1 μM to 1 μM, about 1 μM to 10 μM, about 10 μM to 100 μM, about 100 μM to 1 mM, or about 1 mM to 10 mM and the TPA at a concentration about 100 mM to 4,000 mM.

In some embodiments, the AS 8351 at a concentration of about 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, or 10 mM and the substituted 3-Imidazo[1,2-a]pyridin-3-yl-4-(1,2,3,4-tetrahydro-[1,4]diazepino-[6,7,1-hi]indol-7-yl)pyrrole-2,5-dione, is at a concentration of about 1 μM, 5 μM, 10 μM, 15 μM, 20 μM, 50 μM, 100 μM, 250 μM or 500 μM and the VPA at a concentration about 100 mM to 4,000 mM.

In some embodiments, the pharmaceutical composition comprises a KDM inhibitor that is AS 8351 and a GSK3 Inhibitor that is GSK3-inhibitor XXII and a HDAC inhibitor that is VPA. The AS 8351 is at a concentration of about 0.01 μM to 1000 mM, about 0.1 μM to 100 mM, about 1 μM to 10 mM, about 10 μM to 1000 μM, about 1 μM to 10 μM, 10 μM to 100 μM, about 100 μM to 1000 μM or about 1 mM to 10 mM and the GSK3-inhibitor XXII, at a concentration of about of about 0.1 μM to 1,000 mM, about 1 μM to 100 mM, about 10 μM to 10 mM, about 0.1 μM to 1 μM, about 1 μM to 10 μM, about 10 μM to 100 μM, about 100 μM to 1 mM, about 1 mM to 10 mM, about 10 mM to 100 mM, or about 100 mM to 1000 mM and the VPA at a concentration about 100 mM to 4,000 mM.

In some embodiments, the AS 8351 at a concentration of about 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, or 10 mM and the GSK3-inhibitor XXII is at a concentration of about 0.1 mM, 0.2 mM, 0.3 mM, 0.4 mM, 0.5 mM, 0.6 mM, 0.7 mM, 0.8 mM, 0.9 mM, or 1.0 mM and the VPA at a concentration about 100 mM to 4,000 mM.

In some embodiments, the pharmaceutical composition comprises a KDM inhibitor that is AS 8351 and a GSK3 Inhibitor that is CHIR99021 and a HDAC inhibitor that is VPA. The AS 8351 is at a concentration of about 0.01 μM to 1000 mM, about 0.1 μM to 100 mM, about 1 μM to 10 mM, about 10 μM to 1000 μM, about 1 μM to 10 μM, 10 μM to 100 μM, about 100 μM to 1000 μM or about 1 mM to 10 mM and the CHIR99021 at a concentration of about 0.001 mM to 10,000 mM, about 0.01 mM to 1,000 mM, about 0.1 mM to 100 mM, about 0.001 mM to 0.01 mM, about 0.01 mM to 0.1 mM, about 0.1 mM to 1 mM, about 1 mM to 10 mM, about 10 mM to 100 mM, about 100 mM to 1,000 mM, or about 1,000 mM to 10,000 mM and the VPA at a concentration about 100 mM to 4,000 mM.

In some embodiments, the AS 8351 at a concentration of about 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, or 10 mM and the CHIR99021 is at a concentration of about 1 mM, 2 mM, 3 mM, 4 mM, 5 mM, 6 mM, 7 mM, 8 mM, 9 mM, or 10 mM and the VPA at a concentration about 100 mM to 4,000 mM.

In some embodiments, the pharmaceutical composition comprises a KDM inhibitor that is EPT-103182 and a GSK3 Inhibitor that is AZD1080 and a HDAC inhibitor that is VPA. The EPT-103182 is at a concentration of 0.001 μM to 100 mM, about 0.01 μM to 10 mM, about 0.1 μM to 1 mM, about 1 μM to 100 μM, about 1 μM to 10 μM, 10 μM to 100 μM, or about 100 μM to 1 mM and the AZ1090 is at a concentration of about 0.001 mM to 10,000 mM, about 0.01 mM to 1,000 mM, about 0.1 mM to 100 mM, about 0.001 mM to 0.01 mM, about 0.01 mM to 0.1 mM, about 0.1 mM to 1 mM, about 1 mM to 10 mM, about 10 mM to 100 mM, about 100 mM to 1,000 mM, or about 1,000 mM to 10,000 mM and the VPA at a concentration about 100 mM to 4,000 mM.

In some embodiments, the EPT-103182 at a concentration of about 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, or about 1 mM and the AZ1090 is at a a concentration of about 1 mM, 2 mM, 3 mM, 4 mM, 5 mM, 6 mM, 7 mM, 8 mM, 9 mM, or 10 mM and the VPA at a concentration about 100 mM to 4,000 mM.

In some embodiments, the pharmaceutical composition comprises a KDM inhibitor that is EPT-103182 and a GSK3 Inhibitor that is LY2090314 and a HDAC inhibitor that is VPA. The EPT-103182 is at a concentration of 0.001 μM to 100 mM, about 0.01 μM to 10 mM, about 0.1 μM to 1 mM, about 1 μM to 100 μM, about 1 μM to 10 μM, 10 μM to 100 μM, or about 100 μM to 1 mM and the LY2090314 at a concentration of about 0.001 μM to 10 mM, about 0.01 μM to 1 mM, about 0.1 μM to 100 uM, about 0.001 μM to 0.01 μM, about 0.01 μM to 0.1 μM, about 0.1 μM to 1 μM, about 1 μM to 10 μM, about 10 μM to 100 μM, about 100 μM to 1 mM, or about 1 mM to 10 mM and the VPA at a concentration about 100 mM to 4,000 mM.

In some embodiments, the EPT-103182 at a concentration of about 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, or about 1 mM and the LY2090314 the is at a concentration of about 1 μM, 5 μM, 10 μM, 15 μM, 20 μM or 40 μM and the VPA at a concentration about 100 mM to 4,000 mM.

In some embodiments, the pharmaceutical composition comprises a KDM inhibitor that is EPT-103182 and a GSK3 Inhibitor that is that is a substituted 3-Imidazo[1,2-a]pyridin-3-yl-4-(1,2,3,4-tetrahydro-[1,4]diazepino-[6,7,1-hi]indol-7-yl)pyrrole-2,5-dione and a HDAC inhibitor that is VPA. The EPT-103182 is at a concentration of 0.001 μM to 100 mM, about 0.01 μM to 10 mM, about 0.1 μM to 1 mM, about 1 μM to 100 μM, about 1 μM to 10 μM, 10 μM to 100 μM, or about 100 μM to 1 mM and the substituted 3-Imidazo[1,2-a]pyridin-3-yl-4-(1,2,3,4-tetrahydro-[1,4]diazepino-[6,7,1-]indol-7-yl)pyrrole-2,5-dione at a concentration of about 0.001 μM to 10 mM, about 0.01 μM to 1 mM, about 0.1 μM to 100 uM, about 0.001 μM to 0.01 μM, about 0.01 μM to 0.1 μM, about 0.1 μM to 1 μM, about 1 μM to 10 μM, about 10 μM to 100 μM, about 100 μM to 1 mM, or about 1 mM to 10 mM and the VPA at a concentration about 100 mM to 4,000 mM.

In some embodiments, the EPT-103182 at a concentration of about 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, or about 1 mM and the substituted 3-Imidazo[1,2-a]pyridin-3-yl-4-(1,2,3,4-tetrahydro-[1,4]diazepino-[6,7,1-hi]indol-7-yl)pyrrole-2,5-dione, is at a concentration of about 1 μM, 5 μM, 10 μM, 15 μM, 20 μM, 50 μM, 100 μM, 250 μM or 500 μM and the VPA at a concentration about 100 mM to 4,000 mM.

In some embodiments, the pharmaceutical composition comprises a KDM inhibitor that is EPT-103182 and a GSK3 Inhibitor that is GSK3-inhibitor XXII and a HDAC inhibitor that is VPA. The EPT-103182 is at a concentration of 0.001 μM to 100 mM, about 0.01 μM, to 10 mM, about 0.1 μM to 1 mM, about 1 μM to 100 μM, about 1 μM to 10 μM, 10 μM to 100 μM, or about 100 μM to 1 mM and the GSK3-inhibitor XXII, at a concentration of about of about 0.1 μM to 1,000 mM, about 1 μM to 100 mM, about 10 μM to 10 mM, about 0.1 μM to 1 μM, about 1 μM to 10 μM, about 10 μM to 100 μM, about 100 μM to 1 mM, about 1 mM to 10 mM, about 10 mM to 100 mM, or about 100 mM to 1000 mM and the VPA at a concentration about 100 mM to 4,000 mM.

In some embodiments, the EPT-103182 at a concentration of about 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, or about 1 mM and the GSK3-inhibitor XXII is at a concentration of about 0.1 mM, 0.2 mM, 0.3 mM, 0.4 mM, 0.5 mM, 0.6 mM, 0.7 mM, 0.8 mM, 0.9 mM, or 1.0 mM and the VPA at a concentration about 100 mM to 4,000 mM.

In some embodiments, the pharmaceutical composition comprises a KDM inhibitor that is EPT-103182 and a GSK3 Inhibitor that is CHIR99021 and a HDAC inhibitor that is VPA. The EPT-103182 is at a concentration of 0.001 μM to 100 mM, about 0.01 μM to 10 mM, about 0.1 μM to 1 mM, about 1 μM to 100 μM, about 1 μM to 10 μM, 10 μM to 100 μM, or about 100 μM to 1 mM and the CHIR99021 at a concentration of about 0.001 mM to 10,000 mM, about 0.01 mM to 1,000 mM, about 0.1 mM to 100 mM, about 0.001 mM to 0.01 mM, about 0.01 mM to 0.1 mM, about 0.1 mM to 1 mM, about 1 mM to 10 mM, about 10 mM to 100 mM, about 100 mM to 1,000 mM, or about 1,000 mM to 10,000 mM and the VPA at a concentration about 100 mM to 4,000 mM.

In some embodiments, the EPT-103182 at a concentration of about 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, or about 1 mM and the CHIR99021 is at a concentration of about 1 mM, 2 mM, 3 mM, 4 mM, 5 mM, 6 mM, 7 mM, 8 mM, 9 mM, or 10 mM and the VPA at a concentration about 100 mM to 4,000 mM.

In some embodiments, as noted above, a composition is adapted for administration to the inner ear and/or middle ear, for example, local administration to the round window membrane or intratympanic or transtympanic administration, for example, to cochlear tissue. Alternatively, as noted above, a composition is adapted for administration systemically for example, orally or parentally.

When administered locally, for example to the inner and/or middle ear, the compounds(s) are administered at a unit dose of about 25 μl to 500 μl, or about 50 μl to 200 μl.

The phrase “pharmaceutically-acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used herein “pharmaceutically-acceptable carrier, diluent or excipient” includes without limitation any adjuvant, carrier, excipient, glidant, sweetening agent, diluent, preservative, dye/colorant, flavor enhancer, surfactant, wetting agent, dispersing agent, suspending agent, stabilizer, isotonic agent, solvent, surfactant, or emulsifier which has been approved by the United States Food and Drug Administration as being acceptable for use in humans or domestic animals. Exemplary pharmaceutically-acceptable carriers include, but are not limited to, to sugars, such as lactose, glucose and sucrose; starches, such as corn starch and potato starch; cellulose, and its derivatives, such as sodium carboxymethyl cellulose, ethyl cellulose and cellulose acetate; tragacanth; malt; gelatin; talc; cocoa butter, waxes, animal and vegetable fats, paraffins, silicones, bentonites, silicic acid, zinc oxide; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; and any other compatible substances employed in pharmaceutical formulations.

Certain compositions comprise at least one biocompatible matrix. The term “biocompatible matrix” as used herein is a polymeric carrier that is acceptable for administration to humans for the release of therapeutic agents. In some instances, a biocompatible matrix is a biocompatible gel, foam, fiber, film, or mats. In some embodiments the biocompatible matrix is derived from silk.

In some embodiments the biocompatible matrix comprises hyaluronic acid, hyaluronates, lecithin gels, pluronics, poly(ethyleneglycol), polymers, poloxamers, chitosans, xyloglucans, collagens, fibrins, polyesters, poly(lactides), poly(glycolide), polylactic-co-glycolic acid (PLGA), sucrose acetate isobutyrate, glycerol monooleate, poly anhydrides, poly caprolactone sucrose, glycerol monooleate or a combination thereof.

Exemplary polymers suitable for formulating the biologically active compositions of the present disclosure include, but are not limited to polyamides, polycarbonates, polyalkylenes (polyethylene glycol (PEG)), polymers of acrylic and methacrylic esters, polyvinyl polymers, polyglycolides, polysiloxanes, polyurethanes and co-polymers thereof, celluloses, polypropylene, polyethylenes, polystyrene, polymers of lactic acid and glycolic acid, polyanhydrides, poly(ortho)esters, poly(butic acid), poly(valeric acid), poly(lactide-co-caprolactone), polysaccharides, proteins, polyhyaluronic acids, polycyanoacrylates, and blends, mixtures, or copolymers thereof.

In some embodiments, the polymer is in a concentration between about 5 wt % and about 25 wt % relative to the composition, or about 5 wt %, 6 wt %, 7 wt %, 8 wt %, 9 wt %, 10 wt %, 11 wt %, 12 wt %, 13 wt %, 14 wt %, 15 wt %, 16 wt %, 17 wt %, 18 wt %, 19 wt %, 20 wt %, 21 wt %, 22 wt %, 23 wt %, 24 wt %, or 25 wt % relative to the composition. In certain embodiments, the polymer is in a concentration between about 10 wt % and about 23 wt % relative to the composition. In some embodiments the polymer is in a concentration between about 15 wt % and about 20 wt % relative to the composition. In particular embodiments, the polymer is in a concentration is approximately 17 wt % relative to the composition.

In one embodiment, a biologically active composition of the present disclosure is formulated in a ABA-type or BAB-type triblock copolymer or a mixture thereof, wherein the A-blocks are relatively hydrophobic and comprise biodegradable polyesters or poly(orthoester), and the B-blocks are relatively hydrophilic and comprise polyethylene glycol (PEG). The biodegradable, hydrophobic A polymer block comprises a polyester or poly(ortho ester), in which the polyester is synthesized from monomers selected from the group consisting of D,L-lactide, D-lactide, L-lactide, D,L-lactic acid, D-lactic acid, L-lactic acid, glycolide, glycolic acid, ε-caprolactone, ε-hydroxyhexanoic acid, γ-butyrolactone, γ-hydroxybutyric acid, δ-valerolactone, δ-hydroxyvaleric acid, hydroxybutyric acids, malic acid, and copolymers thereof.

In some embodiments, the copolymer is in a concentration between about 5 wt % and about 25 wt % relative to the composition, or about 5 wt %, 6 wt %, 7 wt %, 8 wt %, 9 wt %, 10 wt %, 11 wt %, 12 wt %, 13 wt %, 14 wt %, 15 wt %, 16 wt %, 17 wt %, 18 wt %, 19 wt %, 20 wt %, 21 wt %, 22 wt %, 23 wt %, 24 wt %, or 25 wt % relative to the composition. In certain embodiments, the copolymer is in a concentration between about 10 wt % and about 23 wt % relative to the composition. In some embodiments the copolymer is in a concentration between about 15 wt % and about 20 wt % relative to the composition. In particular embodiments, the copolymer is in a concentration is approximately 17 wt % relative to the composition.

Certain compositions comprise at least one poloxamer. Poloxamers are triblock copolymers formed of (i.e. hydrophilic poly(oxyethylene) blocks and hydrophobic poly(oxypropylene) blocks) configured as a triblock of poly(oxyethylene)-poly(oxypropylene)-poly(oxyethylene). Poloxamers are one class of block copolymer surfactants having a propylene oxide block hydrophobe and an ethylene oxide hydrophile. Poloxamers are commercially available (e.g. Pluronic® polyols are available from BASF Corporation). Alternatively, poloxamers can be synthesized by known techniques.

Exemplary poloxamers include Poloxamer 124, Poloxamer 188, Poloxamer 237, Poloxamer 338, and Poloxamer 407. In some embodiments, the poloxamer comprises mixtures of two or more of Poloxamer 124, Poloxamer 188, Poloxamer 237, Poloxamer 338 or Poloxamer 407. In some embodiments, the mixture of two or more poloxamers comprise Poloxamer 407 and Poloxamer 124. In certain embodiments the poloxamer comprises at least one of Poloxamer 188 and Poloxamer 407 or mixtures thereof. In some embodiments, the poloxamer is Poloxamer 407.

In some embodiments, the poloxamer is in a concentration between about 5 wt % and about 25 wt % relative to the composition, or about 5 wt %, 6 wt %, 7 wt %, 8 wt %, 9 wt %, 10 wt %, 11 wt %, 12 wt %, 13 wt %, 14 wt %, 15 wt %, 16 wt %, 17 wt %, 18 wt %, 19 wt %, 20 wt %, 21 wt %, 22 wt %, 23 wt %, 24 wt %, or 25 wt % relative to the composition. In certain embodiments, the poloxamer is in a concentration between about 10 wt % and about 23 wt % relative to the composition. In some embodiments the poloxamer is in a concentration between about 15 wt % and about 20 wt % relative to the composition. In particular embodiments, the poloxamer is in a concentration is approximately 17 wt % relative to the composition.

In some embodiments, wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Certain compositions comprise at least one antioxidant. Examples of pharmaceutically-acceptable antioxidants include: (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

In specific embodiments, the viscosity of the composition at about body temperature is substantially different (e.g. lesser, greater) than the viscosity of the composition at room temperature.

In some embodiments, the composition comprises a buffer. For example, in certain instances, the buffer is physiological saline or phosphate-buffered saline (PBS).

In some embodiments, the composition is at or near physiological pH. For instance, in some embodiments, the composition has a pH of between about 6 and about 8, including all integers, decimals, and ranges in between, for example, about 6 to about 6.5 to about 7 to about 7.5 to about 8. In specific embodiments, the composition has a pH of about 7.4 (±0.2).

In some aspects, the present disclosure the pharmaceutical compositions are lyophilized comprising one or more agents described herein and a gelling agent.

In some embodiments, the lyophilized pharmaceutical composition is in the form of a lyophilized cake.

In some embodiments, the lyophilized pharmaceutical composition has a higher stability to oxygen and/or light as compared to a comparable pharmaceutical composition comprising one or more solvents.

In some embodiments, the present disclosure provides a reconstituted solution of the lyophilized pharmaceutical compositions.

As used herein, the term “gelling agent” refers to an agent capable of imparting a gel-like or thickening quality to the pharmaceutical composition or reconstituted solution of the present disclosure upon being subjected to a gelling condition (e.g. a particular temperature or temperature range, the presence of an ion, a pH value or range, or a concentration of gelling agent that causes the gelling agent to undergoing a change or transition from low viscosity to high viscosity, or the reverse). In some embodiments, the gelling condition is a particular temperature (e.g. about 26° C., about 27° C., about 28° C., about 29° C., about 30° C., about 31° C., about 32° C., about 33° C., about 34° C., about 35° C. about 36° C., about 37° C., about 38° C., about 39° C., or about 40° C.). In some embodiments, the gelling condition is a particular temperature range (e.g. about 26° C. or higher, about 27° C. or higher, about 28° C. or higher, about 29° C. or higher, about 30° C. or higher, about 31° C. or higher, about 32° C. or higher, about 33° C. or higher, about 34° C. or higher, about 35° C. or higher, about 36° C. or higher, about 37° C. or higher, about 38° C. or higher, about 39° C. or higher, or about 40° C. or higher). In some embodiments, the gelling agent provides a viscosity of between about 1,000 and 10,000,000 centipoise, between about 5,000 and 5,000,000 centipoise, or between about 100,000 and 4,000,000 centipoise, to the pharmaceutical composition or reconstituted solution of the present disclosure. In some embodiments, the gelling agent provides a viscosity of between about 50,000 and 2,000,000 centipoise to the pharmaceutical composition or reconstituted solution of the present disclosure.

In some embodiments, prior to gelling (e.g. at ambient temperature (e.g. between about 20° C. and about 26° C.)), the gelling agent provides a viscosity of less than about 100,000 centipoise, less than about 50,000 centipoise, 20,000 centipoise, less than about 10,000 centipoise, less than about 8,000 centipoise, less than about 7,000 centipoise, less than about 6,000 centipoise, less than about 5,000 centipoise, less than about 4,000 centipoise, less than about 3,000 centipoise, less than about 2,000 centipoise, or less than about 1,000 centipoise to the the pharmaceutical composition or reconstituted solution of the present disclosure.

In some embodiments, upon gelling (e.g. at the temperature of a human body (e.g. between about 35° C. to about 39° C., between about 36° C. to about 38° C., or at about 37° C.)), the gelling agent provides a viscosity of greater than about 1,000 centipoise, greater than about 5,000 centipoise, greater than about 10,000 centipoise, greater than about 20,000 centipoise, greater than about 50,000 centipoise, greater than about 60,000 centipoise, greater than about 70,000 centipoise, greater than about 80,000 centipoise, greater than about 90,000 centipoise, or greater than about 100,000 centipoise.

In some embodiments, upon gelling (e.g. at the temperature of a human body (e.g. between about 36° C. to about 39° C., or at about 37° C.)), the viscosity of the pharmaceutical composition or reconstituted solution of the present disclosure, as measured in units of centipoise, being about 2 fold or greater, about 5 fold or greater, about 10 fold or greater, about 20 fold or greater, about 50 fold or greater, about 60 fold or greater, about 7 fold or greater, about 80 fold or greater, about 90 fold or greater, about 100 fold or greater as compared to the viscosity of the pharmaceutical composition or reconstituted solution prior to gelling (e.g. at ambient temperature (e.g. at about 25° C.)).

It is understood that the gelling condition (e.g. gelling temperature) of the pharmaceutical composition or reconstituted solution of the present disclosure is measured with a variety of techniques in the art. In some embodiment, the gelling temperature is determined using a commercially available rheometer having a parallel plate geometry (e.g. with plate distance ranging from 0.5 mm to 1.0 mm). In some embodiments, the analysis is performed over a continuous temperature range (e.g. 15° C. to 40° C.) at a constant rate (e.g. 2 to 3° C./min) and a deformation frequency of 0.74 Hz to 1 Hz. The geleation temperature is determined at the temperature whereby the shear storage modulus (G′) and the shear loss modulus (G″) are equal.

In some embodiments, the gelling agent comprises acacia, alginic acid, bentonite, poly(acrylic acid) (Carbomer), carboxymethyl cellulose, ethylcellulose, gelatin, hydroxyethyl cellulose, hydroxypropyl cellulose, magnesium aluminum silicate (Veegum), methylcellulose, poloxamer, hyaluronic acid sodium, polylacticglycolic acid sodium, chitosan, polyvinyl alcohol, sodium alginate, tragacanth, xanthan gum, or any combination thereof. In some embodiment, the gelling agent comprises poloxamer.

In some embodiments, the gelling agent is a thermoreversible gelling agent.

As used herein, the term “thermoreversible” refers to a capability of being reversible by the application of heat. The “thermoreversible gelling agent” refers to an agent capable of reversibly imparting a gel-like or thickening quality to the pharmaceutical composition or reconstituted solution of the present disclosure upon application of heat.

In some embodiments, the thermoreversible gelling agent comprises a poloxamer.

It is understood that the gelling agent (e.g. the thermoreversible gelling agent) may also be a bulking agent of the pharmaceutical composition or reconstituted solution of the present disclosure. In some embodiments, a poloxamer (e.g. poloxamer 407) is the gelling agent and/or the bulking agent of the pharmaceutical composition or reconstituted solution of the present disclosure. Poloxomers are a general class of commercially available and pharmaceutically acceptable triblock copolymers of polyethylene oxide-polypropylene oxide-polyethylene oxide which exhibit relatively low viscosity at low temperatures (e.g. room temperature or below) but much high viscosities at elevated temperatures (e.g. body temperatures of approximately 37° C.) whereby compositions containing such thermoreversible gelling agents effectively solidify in place. Other thermoreversible gelling agents such as polyethylene oxide-polylactic acid-polyethylene oxide polymers are also suitable in various embodiments of the present invention.

In some embodiments, the poloxamer (e.g. poloxamer 407) is the gelling agent and the bulking agent of the pharmaceutical composition or reconstituted solution of the present disclosure. In some embodiments, the presence of the poloxamer (e.g. poloxamer 407) in the pharmaceutical composition (e.g. the lyophilized pharmaceutical composition) alleviates the need for any other excipient (e.g. additional bulking agent). Such alleviation may provide one or more advantages to the pharmaceutical composition (e.g. enhanced stability and/or reduced reconstitution time).

In some embodiments, the poloxamer is selected from the group consisting of Poloxamer 101, Poloxamer 105, Poloxamer 108, Poloxamer 122, Poloxamer 123, Poloxamer 124, Poloxamer 181, Poloxamer 182,Poloxamer 183, Poloxamer 184, Poloxamer 185, Poloxamer 188, Poloxamer 212, Poloxamer 215, Poloxamer 217, Poloxamer 231, Poloxamer 234, Poloxamer 235, Poloxamer 237, Poloxamer 238, Poloxamer 282, Poloxamer 284, Poloxamer 288, Poloxamer 331, Poloxamer 333, Poloxamer 334, Poloxamer 335, Poloxamer 338, Poloxamer 401, Poloxamer 402, Poloxamer 403, and Poloxamer 407.

In some embodiments, the poloxamer is Poloxamer 188 or Poloxamer 407.

In some embodiments, the the poloxamer is Poloxamer 407.

In some embodiments, the poloxamer is a purified poloxamer (e.g. purified. Poloxamer 407).

In some embodiments, the purified poloxamer (e.g. purified Poloxamer 407) has an average molecular weight of about 9 kDa or greater, about 9.2 kDa or greater, about 9.4 kDa or greater, about 9.6 kDa or greater, about 9.8 kDa or greater, about 10 kDa or greater, about 10.2 kDa or greater, about 10.4 kDa or greater, about 10.6 kDa or greater, about 10.8 kDa or greater, about 11 kDa or greater, about 11.2 kDa or greater, about 11.4 kDa or greater, about 11.6 kDa or greater, about 11.8 kDa or greater, about 12 kDa or greater, or about 12.1 kDa or greater.

In some embodiments, the purified poloxamer (e.g. purified Poloxamer 407) has a reduced level of polymer chains with molecular weight below 9 kDa as compared to the unpurified poloxamer (e.g. unpurified Poloxamer 407).

In some embodiments, the purified poloxamer (e.g. purified Poloxamer 407) has about 99% or less, about 98% or less, about 95% or less, about 90% or less, about 80% or less, about 70% or less, about 60% or less, about 50% or less, about 40% or less, about 30% or less, about 20% or less, or about 10% or less of polymer chains with molecular weight below 9 kDa as compared to the unpurified poloxamer (e.g. unpurified. Poloxamer 407).

In some embodiments, the purified poloxamer (e.g. purified Poloxamer 407) is prepared by liquid-liquid extraction or size exclusion chromatography.

In some embodiments, about 10% or more, about 20% or more, about 30% or more, about 40% or more, about 50% or more, about 60% or more, about 70% or more, about 80% or more, about 90% or more, about 95% or more, about 98% or more, or about 99% or more of the one or more impurities having molecular weights below 9 kDa are removed from the poloxamer (e.g. Poloxamer 407) during the purification.

In some embodiments, about 10% or more, about 20% or more, about 30% or more, about 40% or more, about 50% or more, about 60% or more, about 70% or more, about 80% or more, about 90% or more, about 95% or more, about 98% or more, or about 99% or more of the one or more diblock copolymers (e.g. PEO-PPO), single block polymers (e.g. PEO), and/or aldehydes are removed from the poloxamer (e.g. Poloxamer 407) during the purification.

In some embodiments, the pharmaceutical composition, pharmaceutical composition, the lyophilized pharmaceutical composition or reconstituted solution of the present disclosure comprises a buffering agent. The buffer controls the pH of the reconstituted solution to a range of from about 4 to about 13, from about 5 to about 12, from about 6 to about 11, from about 6.5 to about 10.5, or from about 7 to about 10.

Examples of the buffering agent include, but are not limited to, citrate buffering agents, acetate buffering agents, phosphate buffering agents, ammonium chloride, calcium carbonate, calcium chloride, calcium citrate, calcium glubionate, calcium gluceptate, calcium gluconate, d-gluconic acid, calcium glycerophosphate, calcium lactate, calcium lactobionate, propanoic acid, calcium levulinate, pentanoic acid, dibasic calcium phosphate, phosphoric acid, tribasic calcium phosphate, calcium hydroxide phosphate, potassium acetate, potassium chloride, potassium gluconate, potassium mixtures, dibasic potassium phosphate, monobasic potassium phosphate, potassium phosphate mixtures, sodium acetate, sodium bicarbonate, sodium chloride, sodium citrate, sodium lactate, dibasic sodium phosphate, monobasic sodium phosphate, sodium phosphate mixtures, tromethamine, amino-sulfonate buffers (e.g. HEPES), magnesium hydroxide, aluminum hydroxide, alginic acid, pyrogen-free water, isotonic saline, Ringer's solution, ethyl alcohol, and/or combinations thereof. Lubricating agents are selected from the non-limiting group consisting of magnesium stearate, calcium stearate, stearic acid, silica, talc, malt, glyceryl behenate, hydrogenated vegetable oils, polyethylene glycol, sodium benzoate, sodium acetate, sodium chloride, leucine, magnesium lauryl sulfate, sodium lauryl sulfate, and combinations thereof.

In some embodiments, the buffering agent comprises phosphate buffered saline, tris acetate, tris HCl-65, sodium citrate, histidine, arginine, sodium phosphate, tris base-65, hydroxyethyl starch, or any combination thereof.

In some embodiments, the pharmaceutical composition, pharmaceutical composition, the lyophilized pharmaceutical composition or reconstituted solution of the present disclosure comprises a bulking agent.

In some embodiments, the bulking agent comprises poloxamer (e.g. poloxamer 407), mannitol, sucrose, maltose, trehalose, dextrose, sorbitol, glucose, raffinose, glycine, histidine, polyvinylpyrrolidone (e.g. polyvinylpyrrolidone K12 or polyvinylpyrrolidone K17), lactose, or any combination thereof.

In some embodiments, the pharmaceutical composition, pharmaceutical composition, the lyophilized pharmaceutical composition or reconstituted solution of the present disclosure comprises a stabilizing agent.

In some embodiments, the stabilizing agent comprises a cryoprotectant. In some embodiments, the cryoprotectant is a polyol (e.g. a diol or a triol such as propylene glycol (i.e. 1,2-propanediol), 1,3-propanediol, glycerol, (+/−)-2-methyl-2,4-pentanediol, 1,6-hexanediol, 1,2-butanediol, 2,3-butanediol, ethylene glycol, or diethylene glycol), a nondetergent sulfobetaine (e.g. NDSB-201 (3-(1-pyridino)-1-propane sulfonate), an osmolyte (e.g. L-proline or trimethylamine N-oxide dihydrate), a polymer (e.g. polyethylene glycol 200 (PEG 200), PEG 400, PEG 600, PEG 1000, PEG 3350, PEG 4000, PEG 8000, PEG 10000, PEG 20000, polyethylene glycol monomethyl ether 550 (mPEG 550), mPEG 600, mPEG 2000, mPEG 3350, mPEG 4000, mPEG 5000, polyvinylpyrrolidone (e.g. polyvinylpyrrolidone K 15), pentaerythritol propoxylate, or polypropylene glycol P 400), an organic solvent (e.g. dimethyl sulfoxide (DMSO) or ethanol), a sugar (e.g. D-(+)-sucrose, D-sorbitol, trehalose, D-(+)-maltose monohydrate, meso-erythritol, xylitol, myo-inositol, D-(+)-raffinose pentahydrate, D-(+)-trehalose dihydrate, or D-(+)-glucose monohydrate), or a salt (e.g. lithium acetate, lithium chloride, lithium formate, lithium nitrate, lithium sulfate, magnesium acetate, sodium chloride, sodium formate, sodium malonate, sodium nitrate, sodium sulfate, or any hydrate thereof) or any combination thereof.

In some embodiments, the stabilizing agent comprises a salt. In some embodiment, the salt is selected from the group consisting of lithium salts (e.g. lithium acetate, lithium chloride, lithium formate, lithium nitrate, lithium sulfate, or any hydrate thereof), magnesium salts (e.g. magnesium acetate or a hydrate thereof), and sodium salts (e.g. sodium chloride, sodium formate, sodium malonate, sodium nitrate, sodium sulfate, or any hydrate thereof). For another example, the formulation comprises one or more sodium salts. For yet another example, the formulation comprises sodium chloride.

In some embodiment, the stabilizing agent comprises a surfactant. In some embodiments, the surfactant comprises one or more anionic surfactants (e.g. 2-acrylamido-2-methylpropane sulfonic acid, ammonium lauryl sulfate, ammonium perfluorononanoate, docusate, disodium cocoamphodiacetate, magnesium laureth sulfate, perfluorobutanesulfonic acid, perfluorononanoic acid, perfluorooctanesulfonic acid, perfluorooctanoic acid, potassium lauryl sulfate, sodium alkyl sulfate, sodium dodecyl sulfate, sodium dodecylbenzenesulfonate, sodium laurate, sodium laureth sulfate, sodium lauroyl sarcosinate, sodium myreth sulfate, sodium nonanoyloxybenzenesulfonate, sodium pareth sulfate, sodium stearate, or sulfolipid), one or more cationic surfactants (e.g. behentrimonium chloride, benzalkonium chloride, benzethonium chloride, benzododecinium bromide, bronidox, carbethopendecinium bromide, cetalkonium chloride, cetrimonium bromide, cetrimonium chloride, cetylpyridinium chloride, didecyldimethylammonium chloride, dimethyldioctadecylammonium bromide, dimethyldioctadecylammonium chloride, domiphen bromide, lauryl methyl gluceth-10 hydroxypropyl dimonium chloride, octenidine dihydrochloride, olaflur, n-oleyl-1,3-propanediamine, pahutoxin, stearalkonium chloride, tetramethylammonium hydroxide, or thonzonium bromide), one or more zwitterionic surfactants (e.g. cocamidopropyl betaine, cocamidopropyl hydroxysultaine, dipalmitoylphosphatidylcholine, egg lecithin, hydroxysultaine, lecithin myristamine oxide, peptitergents, or sodium lauroamphoacetate), and/or one or more non-ionic surfactants (e.g. alkyl polyglycoside, cetomacrogol 1000, cetostearyl alcohol, cetyl alcohol, cocamide dea, cocamide mea, decyl glucoside, decyl polyglucose, glycerol monostearate, igepal ca-630, isoceteth-20, lauryl glucoside, maltosides, monolaurin, mycosubtilin, narrow-range ethoxylate, nonidet p-40, nonoxynol-9, nonoxynols, np-40, octaethylene glycol monododecyl ether, n-octyl beta-d-thioglucopyranoside, octyl glucoside, oleyl alcohol, peg-10 sunflower glycerides, pentaethylene glycol monododecyl ether, polidocanol, α-tocopheryl polyethylene glycol succinate (TPGS), poloxamer (e.g. poloxamer 407), polyethoxylated tallow amine, polyglycerol polyricinoleate, polysorbate (e.g. polysorbate 20, polysorbate 40, polysorbate 60, or polysorbate 80), sorbitan, sorbitan monolaurate, sorbitan monostearate, sorbitan tristearate, stearyl alcohol, surfactin, triton x-100).

In some embodiments, the pharmaceutical composition, pharmaceutical composition, the lyophilized pharmaceutical composition or reconstituted solution of the present disclosure comprises a tonicity-adjusting agent.

In some embodiments, the tonicity-adjusting agent comprises NaCl, dextrose, dextran, ficoll, gelatin, mannitol, sucrose, glycine, glycerol, or any combination thereof.

In some embodiments, the the pharmaceutical composition or reconstituted solution of the present disclosure comprises a soothing agent. In some embodiments, the soothing agent comprises lidocaine

In addition to these components, the pharmaceutical composition, pharmaceutical composition, the lyophilized pharmaceutical composition or reconstituted solution of the present disclosure includes any substance useful in pharmaceutical compositions.

In some embodiments, the pharmaceutical composition, pharmaceutical composition, the lyophilized pharmaceutical composition or reconstituted solution of the present disclosure includes one or more pharmaceutically acceptable excipients or accessory ingredients such as, but not limited to, one or more solvents, dispersion media, diluents, dispersion aids, suspension aids, granulating aids, disintegrants, fillers, glidants, liquid vehicles, binders, surface active agents, isotonic agents, thickening or emulsifying agents, buffering agents, lubricating agents, oils, preservatives, and other species. Excipients such as waxes, butters, coloring agents, coating agents, flavorings, and perfuming agents may also be included. Pharmaceutically acceptable excipients are well known in the art (see for example Remington's The Science and Practice of Pharmacy, 21^{st} Edition, A. R. Gennaro; Lippincott, Williams & Wilkins, Baltimore, Md., 2006).

Examples of diluents may include, but are not limited to, calcium carbonate, sodium carbonate, calcium phosphate, dicalcium phosphate, calcium sulfate, calcium hydrogen phosphate, sodium phosphate lactose, sucrose, cellulose, microcrystalline cellulose, kaolin, mannitol, sorbitol, inositol, sodium chloride, dry starch, cornstarch, powdered sugar, and/or combinations thereof. Granulating and dispersing agents are selected from the non-limiting list consisting of potato starch, corn starch, tapioca starch, sodium starch glycolate, clays, alginic acid, guar gum, citrus pulp, agar, bentonite, cellulose and wood products, natural sponge, cation-exchange resins, calcium carbonate, silicates, sodium carbonate, cross-linked poly(vinyl-pyrrolidone) (crospovidone), sodium carboxymethyl starch (sodium starch glycolate), carboxymethyl cellulose, cross-linked sodium carboxymethyl cellulose (croscarmellose), methylcellulose, pregelatinized starch (starch 1500), microcrystalline starch, water insoluble starch, calcium carboxymethyl cellulose, magnesium aluminum silicate (VEEGUM®), sodium lauryl sulfate, quaternary ammonium compounds, and/or combinations thereof.

Surface active agents and/or emulsifiers may include, but are not limited to, natural emulsifiers (e.g. acacia, agar, alginic acid, sodium alginate, tragacanth, chondrux, cholesterol, xanthan, pectin, gelatin, egg yolk, casein, wool fat, cholesterol, wax, and lecithin), colloidal clays (e.g. bentonite [aluminum silicate] and VEEGUM® [magnesium aluminum silicate]), long chain amino acid derivatives, high molecular weight alcohols (e.g. stearyl alcohol, cetyl alcohol, oleyl alcohol, triacetin monostearate, ethylene glycol distearate, glyceryl monostearate, and propylene glycol monostearate, polyvinyl alcohol), carbomers (e.g. carboxy polymethylene, polyacrylic acid, acrylic acid polymer, and carboxyvinyl polymer), carrageenan, cellulosic derivatives (e.g. carboxymethylcellulose sodium, powdered cellulose, hydroxymethyl cellulose, hydroxypropyl cellulose, hydroxypropyl methylcellulose, methylcellulose), sorbitan fatty acid esters (e.g. polyoxyethylene sorbitan monolaurate [TWEEN®20], polyoxyethylene sorbitan [TWEEN® 60], polyoxyethylene sorbitan monooleate [TWEEN®80], sorbitan monopalmitate [SPAN®40], sorbitan monostearate [SPAN®60], sorbitan tristearate [STAN®65], glyceryl monooleate, sorbitan monooleate [STAN®80]), polyoxyethylene esters (e.g. polyoxyethylene monostearate [MYRJ® 45], polyoxyethylene hydrogenated castor oil, polyethoxylated castor oil, polyoxymethylene stearate, and SOLUTOL®), sucrose fatty acid esters, polyethylene glycol fatty acid esters (e.g. CREMOPHOR®), polyoxyethylene ethers, (e.g. polyoxyethylene lauryl ether [BRIJ® 30]), poly(vinyl-pyrrolidone), diethylene glycol monolaurate, triethanolamine oleate, sodium oleate, potassium oleate, ethyl oleate, oleic acid, ethyl laurate, sodium lauryl sulfate, PLURONIC®F 68, POLOXAMER® 188, cetrimonium bromide, cetylpyridinium chloride, benzalkonium chloride, docusate sodium, and/or combinations thereof.

A binding agent is starch (e.g. cornstarch and starch paste); gelatin; sugars (e.g. sucrose, glucose, dextrose, dextrin, molasses, lactose, lactitol, mannitol); natural and synthetic gums (e.g. acacia, sodium alginate, extract of Irish moss, panwar gum, ghatti gum, mucilage of isapol husks, carboxymethylcellulose, methylcellulose, ethylcellulose, hydroxyethylcellulose, hydroxypropyl cellulose, hydroxypropyl methylcellulose, microcrystalline cellulose, cellulose acetate, poly(vinyl-pyrrolidone), magnesium aluminum silicate (VEEGUM®), and larch arabogalactan); alginates; polyethylene oxide; polyethylene glycol; inorganic calcium salts; silicic acid; polymethacrylates; waxes; water; alcohol; and combinations thereof, or any other suitable binding agent.

Examples of preservatives may include, but are not limited to, antioxidants, chelating agents, antimicrobial preservatives, antifungal preservatives, alcohol preservatives, acidic preservatives, and/or other preservatives. Examples of antioxidants include, but are not limited to, alpha tocopherol, ascorbic acid, ascorbyl palmitate, butylated hydroxyanisole, butylated hydroxytoluene, monothioglycerol, potassium metabisulfite, propionic acid, propyl gallate, sodium ascorbate, sodium bisulfite, sodium metabisulfite, and/or sodium sulfite. Examples of chelating agents include ethylenediaminetetraacetic acid (EDTA), citric acid monohydrate, disodium edetate, dipotassium edetate, edetic acid, fumaric acid, malic acid, phosphoric acid, sodium edetate, tartaric acid, and/or trisodium edetate. Examples of antimicrobial preservatives include, but are not limited to, benzalkonium chloride, benzethonium chloride, benzyl alcohol, bronopol, cetrimide, cetylpyridinium chloride, chlorhexidine, chlorobutanol, chlorocresol, chloroxylenol, cresol, ethyl alcohol, glycerin, hexetidine, imidurea, phenol, phenoxyethanol, phenylethyl alcohol, phenylmercuric nitrate, propylene glycol, and/or thimerosal. Examples of antifungal preservatives include, but are not limited to, butyl paraben, methyl paraben, ethyl paraben, propyl paraben, benzoic acid, hydroxybenzoic acid, potassium benzoate, potassium sorbate, sodium benzoate, sodium propionate, and/or sorbic acid. Examples of alcohol preservatives include, but are not limited to, ethanol, polyethylene glycol, benzyl alcohol, phenol, phenolic compounds, bisphenol, chlorobutanol, hydroxybenzoate, and/or phenylethyl alcohol. Examples of acidic preservatives include, but are not limited to, vitamin A, vitamin C, vitamin E, beta-carotene, citric acid, acetic acid, dehydroascorbic acid, ascorbic acid, sorbic acid, and/or phytic acid. Other preservatives include, but are not limited to, tocopherol, tocopherol acetate, deteroxime mesylate, cetrimide, butylated hydroxyanisole (BHA), butylated hydroxytoluene (BHT), ethylenediamine, sodium lauryl sulfate (SLS), sodium lauryl ether sulfate (SLES), sodium bisulfite, sodium metabisulfite, potassium sulfite, potassium metabisulfite, GLYDANT PLUS®, PHENONIP®, methylparaben, GERMALL® 115, GERMABEN®II, NEOLONE™, KATHON™, and/or EUXYL®.

Examples of oils include, but are not limited to, almond, apricot kernel, avocado, babassu, bergamot, black current seed, borage, cade, camomile, canola, caraway, carnauba, castor, cinnamon, cocoa butter, coconut, cod liver, coffee, corn, cotton seed, emu, eucalyptus, evening primrose, fish, flaxseed, geraniol, gourd, grape seed, hazel nut, hyssop, isopropyl myristate, jojoba, kukui nut, lavandin, lavender, lemon, litsea cubeba, macademia nut, mallow, mango seed, meadow foam seed, mink, nutmeg, olive, orange, orange roughy, palm, palm kernel, peach kernel, peanut, poppy seed, pumpkin seed, rapeseed, rice bran, rosemary, safflower, sandalwood, sasquana, savoury, sea buckthorn, sesame, rhea butter, silicone, soybean, sunflower, tea tree, thistle, tsubaki, vetiver, walnut, and wheat germ oils as well as butyl stearate, caprylic triglyceride, capric triglyceride, cyclomethicone, diethyl sebacate, dimethicone 360, simethicone, isopropyl myristate, mineral oil, octyldodecanol, oleyl alcohol, an/or silicone oil.

Compounds or compositions described herein can be formulated in any manner suitable for a desired delivery route, e.g. transtympanic injection, transtympanic wicks and catheters, cochlear implants, and injectable depots. In some instances, compositions or formulations include one or more physiologically-acceptable components, including derivatives or prodrugs, solvates, stereoisomers, racemates, or tautomers thereof with any physiologically acceptable carriers, diluents, and/or excipients.

As noted above, certain compositions are adapted for, and certain methods employ, administration to the middle ear or inner ear, for example, by local administration to the round window membrane. The membrane of the round window is the biological barrier to the inner ear space and represents the major obstacle for the local treatment of hearing impairment. The administered drug must overcome this membrane to reach the inner ear space. The drug can operatively (e.g. injection through the tympanic membrane) be placed locally to the round window membrane and can then penetrate through the round window membrane. Substances that penetrate the round window typically distribute in the perilymph and thus reach the hair cells and supporting cells.

The pharmaceutical compositions or formulations may also contain a membrane penetration enhancer, which supports the passage of the agents mentioned herein through the round window membrane. Accordingly, liquid, gel or foam formulations is used. It is also possible to apply the active ingredient orally or to employ a combination of delivery approaches.

Certain compositions are adapted for, and certain methods employ, administration to the middle ear or inner ear, for example, by intratympanic or transtympanic administration. Intratympanic (IT) delivery of drugs to the ear is increasingly used for both clinical and research purposes. Some groups have applied drugs in a sustained manner using microcatheters and microwicks, while the majority have applied them as single or as repeated IT injections (up to 8 injections over periods of up to 2 weeks).

Intratympanically applied drugs are thought to enter the fluids of the inner ear primarily by crossing the round window (RW) membrane. Calculations show that a major factor controlling both the amount of drug entering the ear and the distribution of drug along the length of the ear is the duration the drug remains in the middle ear space. Single, ‘one-shot’ applications or applications of aqueous solutions for few hours' duration result in steep drug gradients for the applied substance along the length of the cochlea and rapidly declining concentration in the basal turn of the cochlea as the drug subsequently becomes distributed throughout the ear.

Other injection approaches include by osmotic pump, or, by combination with implanted biomaterial, by injection or infusion. Biomaterials that can aid in controlling release kinetics and distribution of drug include hydrogel materials, degradable materials. One class of materials that is used includes in situ gelling materials. All potential materials and methodologies mentioned in references (Almeida H, Amaral M H, Lobao P, Lobo J M, Drug Discov Today 2014;19:400-12; Wise A K, Gillespie L N, J Neural Eng 2012; 9:065002; Surovtseva E V, Johnston A H, Zhang W, et al. Int J Pharmaceut 2012; 424:121-7; Roy S. Glueckert R, Johnston A H, et al., Nanomedicine 2012, 7:55-63; Rivera T, Sanz L, Camarero G, Varela-Nieto I., Curr Drug Deliv 2012; 9:231-42; Pararas E E, Borkholder D A, Borenstein J T, Adv Drug Deliv Rev 2012; 64:1650-60; Li M L, Lee L C, Cheng Y R, et al., IEEE T Bio-Med Eng 2013; 60:2450-60; Lajud S A, Han Z, Chi F L, et al., J Control Release 2013; 166:268-76; Kim D K, Park S N, Park K H, et al., Drug Deliv 2014; Engleder E, Honeder C, Klobasa J, Wirth M, Arnoldner C, Gabor F, Int J Pharmaceut 2014; 471:297-302; Bohl A, Rohm H W, Ceschi P, et al., J Mater Sci Mater Med 2012; 23:2151-62; Hoskison E, Daniel M, Al-Zahid S, Shakesheff K M, Bayston R, Birchall J P, Ther Deliv 2013; 4:115-24; Staecker H, Rodgers B, Expert Opin Drug Deliv 2013; 10:639-50; Pritz C O, Dudas J, Rask-Andersen H, Schrott-Fischer A, Glueckert R, Nanomedicine 2013; 8:1155-72), which are included herein by reference in their entirety. Other materials include collagen or other natural materials including fibrin, gelatin, and decellularized tissues. Gelfoam may also be suitable.

Delivery may also be enhanced via alternate means including but not limited to agents added to the delivered composition such as penetration enhancers, or could be through devices via ultrasound, electroporation, or high-speed jet.

Methods described herein can also be used for inner ear cell types that are produced using a variety of methods know to those skilled in the art including those cell types described in PCT Application No. WO2012103012 A1.

With regard to human and veterinary treatment, the amount of a particular agent(s) that is administered is dependent on a variety of factors, including the disorder being treated and the severity of the disorder; activity of the specific agent(s) employed; the age, body weight, general health, sex and diet of the patient; the time of administration, route of administration, and rate of excretion of the specific agent(s) employed; the duration of the treatment; drugs used in combination or coincidental with the specific agent(s) employed; the judgment of the prescribing physician or veterinarian; and like factors known in the medical and veterinary arts.

The agents described herein are administered in a therapeutically effective amount to a subject in need of treatment. Administration of compositions described herein can be via any of suitable route of administration, for example, by intratympanic administration. Other routes include ingestion, or alternatively parenterally, for example intravenously, intra-arterially, intraperitoneally, intrathecally, intraventricularly, intraurethrally, intrasternally, intracranially, intramuscularly, intranasally, subcutaneously, sublingually, transdermally, or by inhalation or insufflations, or topical by ear instillation for absorption through the skin of the ear canal and membranes of the eardrum. Such administration is as a single or multiple oral dose, defined number of ear drops, or a bolus injection, multiple injections, or as a short- or long-duration infusion. Implantable devices (e.g. implantable infusion pumps) may also be employed for the periodic parenteral delivery over time of equivalent or varying dosages of the particular formulation. For such parenteral administration, the compounds are formulated as a sterile solution in water or another suitable solvent or mixture of solvents. The solution may contain other substances such as salts, sugars (particularly glucose or mannitol), to make the solution isotonic with blood, buffering agents such as acetic, citric, and/or phosphoric acids and their sodium salts, and preservatives.

Compositions described herein can be administered by several methods sufficient to deliver the composition to the inner ear. Delivering a composition to the inner ear includes administering the composition to the middle ear, such that the composition may diffuse across the round window to the inner ear. It also includes administering a composition to the inner ear by direct injection through the round window membrane. Such methods include, but are not limited to auricular administration, by transtympanic wicks or catheters, or parenteral administration, for example, by intraauricular, transtympanic, or intracochlear injection.

In particular embodiments, the compounds, compositions and formulations of the disclosure are locally administered, meaning that they are not administered systemically.

In one embodiment, a syringe and needle apparatus is used to administer compounds or compositions to a subject using auricular administration. A suitably sized needle is used to pierce the tympanic membrane and a wick or catheter comprising the composition is inserted through the pierced tympanic membrane and into the middle ear of the subject. The device is inserted such that it is in contact with the round window or immediately adjacent to the round window. Exemplary devices used for auricular administration include, but are not limited to, transtympanic wicks, transtympanic catheters, round window microcatheters (small catheters that deliver medicine to the round window), and Silverstein Microwicks™ (small tube with a “wick” through the tube to the round window, allowing regulation by subject or medical professional).

In some embodiments, a syringe and needle apparatus is used to administer compounds or compositions to a subject using transtympanic injection, injection behind the tympanic membrane into the middle and/or inner ear. The formulation is administered directly onto the round window membrane via transtympanic injection or is administered directly to the cochlea via intracochlear injection or directly to the vestibular organs via intravestibular injection.

In some embodiments, the delivery device is an apparatus designed for administration of compounds or compositions to the middle and/or inner ear. By way of example only: GYRUS Medical GmbH offers micro-otoscopes for visualization of and drug delivery to the round window niche; Arenberg has described a medical treatment device to deliver fluids to inner ear structures in U.S. Pat. Nos. 5,421,818; 5,474,529; and 5,476,446, each of which is incorporated by reference herein for such disclosure. U.S. patent application Ser. No. 08/874,208, which is incorporated herein by reference for such disclosure, describes a surgical method for implanting a fluid transfer conduit to deliver compositions to the inner ear. U.S. Patent Application Publication 2007/0167918, which is incorporated herein by reference for such disclosure, further describes a combined otic aspirator and medication dispenser for transtympanic fluid sampling and medicament application.

In some embodiments, a compound or composition disclosed herein is administered to a subject in need thereof once. In some embodiments, a compound or composition disclosed herein is administered to a subject in need thereof more than once. In some embodiments, a first administration of a compound or composition disclosed herein is followed by a second, third, fourth, or fifth administration of a compound or composition disclosed herein.

The number of times a compound or composition is administered to an subject in need thereof depends on the discretion of a medical professional, the disorder, the severity of the disorder, and the subject's response to the formulation. In some embodiments, the compound or composition disclosed herein is administered once to a subject in need thereof with a mild acute condition. In some embodiments, a compound or composition disclosed herein is administered more than once to a subject in need thereof with a moderate or severe acute condition. In the case wherein the subject's condition does not improve, upon the doctor's discretion the compound or composition is administered chronically, that is, for an extended period of time, including throughout the duration of the subject's life in order to ameliorate or otherwise control or limit the symptoms of the subject's disease or condition.

In the case wherein the subject's status does improve, upon the doctor's discretion the compound or composition may administered continuously; alternatively, the dose of drug being administered is temporarily reduced or temporarily suspended for a certain length of time (i.e. a “drug holiday”). The length of the drug holiday varies between 2 days and 1 year, including by way of example only, 2 days, 3 days, 4 days, 5 days, 6 days, 7 days, 10 days, 12 days, 15 days, 20 days, 28 days, 35 days, 50 days, 70 days, 100 days, 120 days, 150 days, 180 days, 200 days, 250 days, 280 days, 300 days, 320 days, 350 days, and 365 days. The dose reduction during a drug holiday is from 10%-100%, including by way of example only 10%, 15%, 20%, 25%, 30%, 35%, 40%, 45%, 50%, 55%, 60%, 65%, 70%, 75%, 80%, 85%, 90%, 95%, and 100%.

Once the subject's hearing and/or balance has improved, a maintenance dose can be administered, if necessary. Subsequently, the dosage or the frequency of administration, or both, is optionally reduced, as a function of the symptoms, to a level at which the improved disease, disorder or condition is retained. In certain embodiments, subjects require intermittent treatment on a long-term basis upon any recurrence of symptoms.

Certain embodiments include a pharmaceutical product comprising a sealed packaging and the compound(s) according to the invention in the container. The container size can be optimized to reduce head space in the container after packaging and any head space is filled with an inert gas such as nitrogen. Furthermore, container material of construction can be chosen to minimize the moisture and oxygen ingress inside the container after packaging.

Definitions

In this application, the use of “or” includes “and/or” unless stated otherwise. As used in this application, the term “comprise” and variations of the term, such as “comprising” and “comprises,” are not intended to exclude other additives, components, integers or steps. By “consisting of” is meant including, and limited to, whatever follows the phrase “consisting of.” Thus, the phrase “consisting of” indicates that the listed elements are required or mandatory, and that no other elements are present. By “consisting essentially of” is meant including any elements listed after the phrase and limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether they materially affect the activity or action of the listed elements.

The terms “about” and “approximately” are used as equivalents. Any numerals used in this disclosure with or without about/approximately are meant to cover any normal fluctuations appreciated by one of ordinary skill in the relevant art. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

“Any reference to a compound is also a reference to a pharmaceutically acceptable salt of that compound (regardless of whether or not pharmaceutically acceptable salts are explicitly mentioned). Any compound can be provided for use in the invention in any pharmaceutically acceptable solid form, e.g. salt, solvate, hydrate, polymorph, amorphous material form etc. Any references to a compound also include references to artificially deuterated forms of that compound.

“Activity” refers to biological function mediated by proteins of a cell measured by methods known in the art such as immunostaining and western blotting in conjunction with cellular effects such as proliferation, cellular growth, or cellular gene expression.

“Administration” refers to introducing a substance into a subject. In some embodiments, administration is auricular, intraauricular, intracochlear, intravestibular, or transtympanically, e.g. by injection. In some embodiments, administration is directly to the inner ear, e.g. injection through the round window, otic capsule, or vestibular canals. In some embodiments, administration is directly into the inner ear via a cochlear implant delivery system. In some embodiments, the substance is injected transtympanically to the middle ear. In certain embodiments the substance is administered systemically (e.g. orally or parenterally). In certain embodiments “causing to be administered” refers to administration of a second component after a first component has already been administered (e.g, at a different time and/or by a different actor).

“Agonist” refers to an agent that causes an increase in the expression, levels, and/or activity of a target gene, protein, and/or pathway. In some instances, an agonist directly binds to and activates a target protein. In some instances, an agonist increases the activity of a pathway by binding to and modulating the activity of one or more pathway components, for example, by inhibiting the activity of negative regulator(s) of the pathway, or by activating upstream or downstream regulator(s) of the pathway.

“Auricular administration” refers to a method of using a catheter or wick device to administer a composition across the tympanic membrane to the inner ear of the subject. To facilitate insertion of the wick or catheter, the tympanic membrane are pierced using a suitably sized syringe or pipette. The devices could also be inserted using any other methods known to those of skill in the art, e.g. surgical implantation of the device. In particular embodiments, the wick or catheter device is a stand-alone device, meaning that it is inserted into the ear of the subject and then the composition is controllably released to the inner ear. In other particular embodiments, the wick or catheter device is attached or coupled to a pump or other device that allows for the administration of additional compositions. The pump is automatically programmed to deliver dosage units or are controlled by the subject or medical professional.

“Cell Aggregate” as used herein refers to a body cells in the organ of Corti that have proliferated to form a cluster of a given cell type that is greater than 40 microns in diameter and/or produced a morphology in which greater than 3 cell layers reside perpendicular to the basilar membrane.

“Cell Aggregate” can also refer a process in which cell division creates a body of cells that cause one or more cell types to breach the reticular lamina, or the boundary between endolymph and perilymph.

“Cell Density” as used herein in connection with a specific cell type is the mean number of that cell type per area in a Representative Microscopy Sample. The cell types may include but are not limited to Lgr5+ cells, hair cells, or supporting cells. The Cell Density is assessed with a given cell type in a given organ or tissue, including but not limited to the cochlea or organ of Corti. For instance, the Lgr5+ Cell Density in the organ of Corti is the Cell Density of Lgr5+ cells as measured across the organ of Corti. Typically, supporting cells and Lgr5+ cells will be enumerated by taking cross sections of the organ of Corti. Typically, hair cells will be enumerated by looking down at the surface of the organ of Corti, though cross sections are used in some instances, as described in a Representative Microscopy Sample. Typically, Cell Density of Lgr5+ cells will be measured by analyzing whole mount preparations of the Organ of Corti and counting the number of Lgr5 cells across a given distance along the surface of the epithelia, as described in a Representative Microscopy Sample. Hair cells are identified by their morphological features such as bundles or hair cell specific stains (e.g. Myosin VIIa, Prestin, vGlut3, Pou4f3, Espin, conjugated-Phalloidin, PMCA2, Ribeye, Atoh1, etc.). Lgr5+ cells are identified by specific stains or antibodies (e.g. Lgr5-GFP transgenic reporter, anti-Lgr5 antibody, etc.)

“Cochlear Concentration” as used herein will be the concentration of a given agent as measured through sampling cochlear fluid or tissue. Unless otherwise noted, the sample should contain a substantial enough portion of the cochlear fluid or tissue so that it is approximately representative of the average concentration of the agent in the cochlea. For example, samples are drawn from a vestibular canal, and a series of fluid samples drawn in series such that individual samples are comprised of cochlear fluid in specified portions of the cochlea

“Complementary nucleic acid sequence” refers to a nucleic acid sequence capable of hybridizing with another nucleic acid sequence comprised of complementary nucleotide base pairs.

“Cross-Sectional Cell Density” as used herein in connection with a specific cell type is the mean number of that cell type per area of cross section through a tissue in a Representative Microscopy Sample. Cross sections of the organ of Corti can also be used to determine the number of cells in a given plane. Typically, hair cells Cross-sectional Cell Density will be measured by analyzing whole mount preparations of the organ of Corti and counting the number of hair cells across a given distance in cross sections taken along a portion of the epithelia, as described in a Representative Microscopy Sample. Typically, Cross-sectional Cell Density of Lgr5+ cells will be measured by analyzing whole mount preparations of the organ of Corti and counting the number of Lgr5+ cells across a given distance in cross sections taken along a portion of the epithelia, as described in a Representative Microscopy Sample. Hair cells are identified by their morphological features such as bundles or hair cell specific stains (suitable stains include e.g. Myosin VIIa, Prestin, vGlut3, Pou4f3, conjugated-Phalloidin, PMCA2Atoh1, etc.). Lgr5+ cells are identified by specific stains or antibodies (suitable stains and antibodies include fluorescence in situ hybridization of Lgr5 mRNA, Lgr5-GFP transgenic reporter system, anti-Lgr5 antibodies, etc.).

“Decreasing” or “decreases” refers to decreasing by at least 5%, for example, 5, 6, 7, 8, 9, 10, 15, 20, 25, 30, 35, 40, 45, 50, 55, 60, 65, 70, 75, 80, 85, 90, 95, 99 or 100%, for example, as compared to the level of reference or control.

“Decreasing” or “decreases” also includes decreasing by at least about 1.1-fold, for example, at least about 1.1, 1.2, 1.3, 1.4, 1.5, 1.6, 1.7, 1.8, 1.9, 2, 3, 4, 5, 6, 7, 8, 9, 10, 15, 20, 30, 40, 50, 60, 70, 80, 90, 100, 200, 500, 1000-fold or more, for example, as compared to the level of a reference or control.

“Effective Concentration” is the minimum concentration of a compound that induces at least an 1.1, 1.2, 1.3, 1.4, 1.5, 1.6, 1.7, 1.8, 1.9, 2, 3, 4, 5, 6, 7, 8, 9, 10, 15, 20, 30, 40, 50, 60, 70, 80, 90, 100, 200, 500, 1000-fold or more in gene expression and/or about a 1.5-fold increase in number of Lgr5+ cells in a Stem Cell Proliferation Assay compared to the number of Lgr5+ cells in a Stem Cell Proliferation Assay performed without the compound.

“Effective Release Rate” (mass/time) as used herein is the Effective Concentration (mass/volume)*30 uL/1 hour.

“Eliminate” means to decrease to a level that is undetectable.

“Engraft” or “engraftment” refers to the process of stem or progenitor cell incorporation into a tissue of interest in vivo through contact with existing cells of the tissue. “Epithelial progenitor cell” refers to a multipotent cell which has the potential to become restricted to cell lineages resulting in epithelial cells.

“Epithelial stem cell” refers to a multipotent cell which has the potential to become committed to multiple cell lineages, including cell lineages resulting in epithelial cells.

“Expression” refers to gene levels as measured by the amount of RNA

“HDAC inhibitor” refers to any compound that inhibits the cellular activity of Histone Deacetylase classes I-IV

“Hybridize” refers to pairing to form a double-stranded molecule between complementary nucleotide bases (e.g. adenine (A) forms a base pair with thymine (T), as does guanine (G) with cytosine (C) in DNA) under suitable conditions of stringency. (See, e.g. Wahl, G. M. and S. L. Berger (1987) Methods Enzymol. 152:399; Kimmel, A. R. (1987) Methods Enzymol. 152:507).

An “inhibitor” refers to an agent that causes a decrease in the expression, levels, and/or activity of a target gene, protein, and/or pathway. An “antagonist” is one example of an “inhibitor”.

As used herein, an “inhibitory nucleic acid” is a double-stranded RNA, RNA interference, miRNA, siRNA, shRNA, or antisense molecule, or a portion thereof, or a mimetic thereof, that when administered to a mammalian cell results in a decrease in the expression of a target gene. Typically, a nucleic acid inhibitor comprises at least a portion of a target nucleic acid molecule, or an ortholog thereof, or comprises at least a portion of the complementary strand of a target nucleic acid molecule. In some instances, expression of a target gene is reduced by 10%, 25%, 50%, 75%, or even 90-100%.

“In Vitro Lgr5 activity” refers to the level of expression or activity of Lgr5 in an in vitro population of cells. It is measured, for example, in cells derived from a Lgr5-GFP expressing mouse such as a B6.129P2-Lgr5tml(cre/ERT2)Cle/J mouse (also known as Lgr5-EGFP-IRES-creERT2 or Lgr5-GFP mouse, Jackson Lab Stock No: 008875) by dissociating cells to single cells, staining with propidium iodide (PI), and analyzing the cells using a flow cytometer for Lgr5-GFP expression. Inner ear epithelial cells from wild-type (non-Lgr5-GFP) mice that passing the same culturing and analyzing procedures can be used as a negative control. Typically, two population of cells are shown in the bivariate plot with GFP/FITC as one variable, which include both GFP positive and GFP negative populations. Lgr5+ cells can be identified by gating GFP positive cell population. The percentage of Lgr5+ cells can be measured by gating GFP positive cell population against both GFP negative population and the negative control. The number of Lgr5+ cells can be calculated by multiplying the total number of cells by the percentage of Lgr5-positive cells. For cells derived from non-Lgr5-GFP mice, Lgr5 activity can be measured using an anti-Lgr5 antibody or quantitative-PCR on the Lgr5 gene.

“In Vivo Lgr5 activity” as used herein is the level of expression or activity of Lgr5 in a subject. It is measured, for example, by removing an animal's inner ear and measuring Lgr5 protein or Lgr5 mRNA. Lgr5 protein production can be measured using an anti-Lgr5 antibody to measure fluorescence intensity as determined by imaging cochlear samples, where fluorescence intensity is used as a measure of Lgr5 presence. Western blots can be used with an anti-Lgr5 antibody, where cells can be harvested from the treated organ to determine increases in Lgr5 protein. Quantitative-PCR or RNA in situ hybridization can be used to measure relative changes in Lgr5 mRNA production, where cells can be harvested from the inner ear to determine changes in Lgr5 mRNA. Alternatively, Lgr5 expression can be measured using an Lgr5 promoter driven GFP reporter transgenic system, where the presence or intensity GFP fluoresce can be directly detected using flow cytometry, imaging, or indirectly using an anti-GFP antibody.

“Increasing” or “increases” refers to increasing by at least 5%, for example, 5, 6, 7, 8, 9, 10, 15, 20, 25, 30, 35, 40, 45, 50, 55, 60, 65, 70, 75, 80, 85, 90, 95, 99, 100, 150, 200, 250, 300, 350, 400, 450, or 500% or more, for example, as compared to the level of a reference.

“Increasing” or “increases” also means increases by at least about 1.1-fold, for example, at least about 1.1, 1.2, 1.3, 1.4, 1.5, 1.6, 1.7, 1.8, 1.9, 2, 3, 4, 5, 6, 7, 8, 9, 10, 15, 20, 30, 40, 50, 60, 70, 80, 90, 100, 200, 500, 1000-fold or more, for example, as compared to the level of a reference standard.

“Intraauricular administration” refers to administration of a composition to the middle or inner ear of a subject by directly injecting the composition.

“Intracochlear” administration refers to direct injection of a composition across the tympanic membrane and across the round window membrane into the cochlea.

“Intravestibular” administration refers to direct injection of a composition across the tympanic membrane and across the round window or oval window membrane into the vestibular organs.

“Isolated” refers to a material that is free to varying degrees from components which normally accompany it as found in its native state. “Isolate” denotes a degree of separation from original source or surroundings.

“Lgr5” is an acronym for the Leucine-rich repeat-containing G-protein coupled receptor 5, also known as G-protein coupled receptor 49 (GPR49) or G-protein coupled receptor 67 (GPR67). It is a protein that in humans is encoded by the Lgr5 gene.

“Lgr5 Activity” is defined as the level of activity of Lgr5 in a population of cells. In an in vitro cell population, Lgr5 activity is measured in an in vitro Lgr5 Activity assay. In an in vivo cell population, Lgr5 activity is measured in an in vivo Lgr5 Activity assay.

“Lgr5+ cell” or “Lgr5-positive cell” as used herein is a cell that expresses Lgr5. “Lgr5-cell” or “Lgr5-negative” as used herein is a cell that is not Lgr5+.

“Lineage Tracing” as used herein is using a mouse line that enables fate tracing of any cell that expresses a target gene at the time of reporter induction. This can include hair cell or supporting cells genes (Sox2, Lgr5, MyosinVIIa, Pou4f3, etc.). For example, lineage tracing may use an Lgr5-EGFP-IRES-creERT2 mouse crossed with a reporter mouse, which upon induction, allows one to trace the fate of cells that expressed Lgr5 at the time of induction. By further example, Lgr5 cells can be isolated into single cells and cultured in a Stem Cell Proliferation Assay to generate colonies, then subsequently differentiated in a Differentiation Assay and analyzed for cell fate by staining for hair cell and/or supporting cell proteins and determining the reporter co-localization with either hair cell or supporting cell staining to determine the Lgr5 cells' fate. In addition, lineage tracing can be performed in cochlear explants to track supporting cell or hair cell fate within the intact organ after treatment. For example, Lgr5 cell fate can be determined by isolating the cochlea from a Lgr5-EGFP-IRES-creERT2 mouse crossed with a reporter mouse and inducing the reporter in Lgr5 cells before or during treatment. The organ can then be analyzed for cell fate by staining for hair cell and/or supporting cell proteins and determining the reporter co-localization with either hair cell or supporting cell staining to determine the Lgr5 cells' fate. In addition, lineage tracing can be performed in vivo track supporting cell or hair cell fate within the intact organ after treatment. For example, Lgr5 cell fate can be determined inducing a reporter in an Lgr5-EGFP-IRES-creERT2 mouse crossed with a reporter mouse, treating the animal, then isolating the cochlea. The organ can then be analyzed for cell fate by staining for hair cell and/or supporting cell proteins and determining the reporter co-localization with either hair cell or supporting cell staining to determine the Lgr5 cells' fate. Lineage tracing is performed using alternative reporters of interest as is standard in the art.

“Mammal” refers to any mammal including but not limited to human, mouse, rat, sheep, monkey, goat, rabbit, hamster, horse, cow or pig.

“Mean Release Time” as used herein is the time in which one-half of an agent is released into phosphate buffered saline from a carrier in a Release Assay.

“Native Morphology” as used herein is means that tissue organization largely reflects the organization in a healthy tissue.

“Non-human mammal”, as used herein, refers to any mammal that is not a human.

As used in relevant context herein, the term “number” of cells can be 0, 1, or more cells.

“Organ of Corti” as used herein refers to the sensory epithelia of the cochlea where the sensory cells (inner and outer hair cells) and supporting cells reside.

“Organoid” or “epithelial organoid” refers to a cell cluster or aggregate that resembles an organ, or part of an organ, and possesses cell types relevant to that particular organ.

“Pharmaceutically-acceptable salt” includes both acid and base addition salts.

“Pharmaceutically-acceptable base addition salt” refers to those salts which retain the biological effectiveness and properties of the free acids, which are not biologically or otherwise undesirable. These salts are prepared from addition of an inorganic base or an organic base to the free acid. Salts derived from inorganic bases include, but are not limited to, the sodium, potassium, lithium, ammonium, calcium, magnesium, iron, zinc, copper, manganese, aluminum salts and the like. For example, inorganic salts include, but are not limited to, ammonium, sodium, potassium, calcium, and magnesium salts. Salts derived from organic bases include, but are not limited to, salts of primary, secondary, and tertiary amines, substituted amines including naturally occurring substituted amines, cyclic amines and basic ion exchange resins, such as ammonia, isopropylamine, trimethylamine, diethylamine, triethylamine, tripropylamine, diethanolamine, ethanolamine, deanol, 2-dimethylaminoethanol, 2-diethylaminoethanol, dicyclohexylamine, lysine, arginine, histidine, caffeine, procaine, hydrabamine, choline, betaine, benethamine, benzathine, ethylenediamine, glucosamine, methylglucamine, theobromine, triethanolamine, tromethamine, purines, piperazine, piperidine, N-ethylpiperidine, polyamine resins and the like. Example organic bases used in certain embodiments include isopropylamine, diethylamine, ethanolamine, trimethylamine, dicyclohexylamine, choline, and caffeine.

“Population” of cells refers to any number of cells greater than 1, but in some embodiments is at least 1×103 cells, at least 1×104 cells, at least at least 1×105 cells, at least 1×106 cells, at least 1×107 cells, at least 1×108 cells, at least 1×109 cells, or at least 1×1010 cells.

“Progenitor cell” as used herein refers to a cell that, like a stem cell, has the tendency to differentiate into a specific type of cell, but is already more specific than a stem cell and is pushed to differentiate into its “target” cell.

“Proliferation Period” as used herein is the duration of time in which tissue or cells are exposed to an epigenetic agent alone or in combination with a Wnt agonist.

In certain embodiments, the “purity” of any given agent or compound in a composition is specifically defined. For instance, certain compositions may comprise an agent that is at least 80, 85, 90, 91, 92, 93, 94, 95, 96, 97, 98, 99, or 100% pure, including all decimals in between, as measured, for example and by no means limiting, by high performance liquid chromatography (HPLC), a well-known form of column chromatography used frequently in biochemistry and analytical chemistry to separate, identify, and quantify compounds.

“Reference” means a standard or control condition (e.g. untreated with a test agent or combination of test agents).

“Release Assay” as used herein is a test in which the rate of release of an agent from a Biocompatible Matrix through dialysis membrane to a saline environment. An exemplary Release Assay is performed by placing 30 microliters of a composition in 1 ml Phosphate Buffered Saline inside saline dialysis bag with a suitable cutoff, and placing the dialysis bag within 10 mL of Phosphate Buffered Saline at 37° C. The dialysis membrane size is chosen based on agent size in order to allow the agent being assessed to exit the membrane. For small molecule release, a 3.5-5 kDa cutoff is used. The Release Rate for a composition may change over time and is measured in 1 hour increments.

“Representative Microscopy Sample” as used herein describes a sufficient number of fields of view within a cell culture system, a portion of extracted tissue, or an entire extracted organ that the average feature size or number being measured can reasonably be said to represent the average feature size or number if all relevant fields were measured. For example, in order to assess the hair cell counts at a frequency range on the Organ of Corti, ImageJ software (NIH) can used to measure the total length of cochlear whole mounts and the length of individual counted segments. The total number of inner hair cells, outer hair cells, and supporting cells can be counted in the entire or fraction of any of the four cochlear segments of 1200-1400 μm (apical, mid-apical, mid-basal, and basal) at least 3 fields of view at 100 μm field size would be reasonably considered a Representative Microscopy Sample. A Representative Microscopy sample can include measurements within a field of view, which can be measured as cells per a given distance. A Representative Microscopy sample can be used to assess morphology, such as cell-cell contacts, cochlear architecture, and cellular components (e.g. bundles, synapses).

“Rosette Patterning” is a characteristic cell arrangement in the cochlea in which <5% hair cells are adjacent to other hair cells.

The term “sample” refers to a volume or mass obtained, provided, and/or subjected to analysis. In some embodiments, a sample is or comprises a tissue sample, cell sample, a fluid sample, and the like. In some embodiments, a sample is taken from (or is) a subject (e.g. a human or animal subject). In some embodiments, a tissue sample is or comprises brain, hair (including roots), buccal swabs, blood, saliva, semen, muscle, or from any internal organs, or cancer, precancerous, or tumor cells associated with any one of these. A fluid is, but is not limited to, urine, blood, ascites, pleural fluid, spinal fluid, and the like. A body tissue can include, but is not limited to, brain, skin, muscle, endometrial, uterine, and cervical tissue or cancer, precancerous, or tumor cells associated with any one of these. In an embodiment, a body tissue is brain tissue or a brain tumor or cancer. Those of ordinary skill in the art will appreciate that, in some embodiments, a “sample” is a “primary sample” in that it is obtained from a source (e.g. a subject); in some embodiments, a “sample” is the result of processing of a primary sample, for example to remove certain potentially contaminating components and/or to isolate or purify certain components of interest.

“Self-renewal” refers to the process by which a stem cell divides to generate one (asymmetric division) or two (symmetric division) daughter cells with development potentials that are indistinguishable from those of the mother cell. Self-renewal involves both proliferation and the maintenance of an undifferentiated state.

“siRNA” refers to a double stranded RNA. Optimally, an siRNA is 18, 19, 20, 21, 22, 23 or 24 nucleotides in length and has a 2 base overhang at its 3′ end. These dsRNAs can be introduced to an individual cell or culture system. Such siRNAs are used to downregulate mRNA levels or promoter activity.

“Stem cell” refers to a multipotent cell having the capacity to self-renew and to differentiate into multiple cell lineages.

“Stem Cell Differentiation Assay” as used herein is an assay to determine the differentiation capacity of stem cells. In an exemplary Stem Cell Differentiation Assay, the number of cells for an initial cell population is harvested from a Atoh1-GFP mouse between the age of 3 to 7 days, by isolating the Organ of Corti sensory epithelium, dissociating the epithelium into single cells, and passing the cells through a 40 um cell strainer. Approximately 5000 cells are entrapped in 40 μl of culture substrate (for example: Matrigel (Corning, Growth Factor Reduced)) and placed at the center of wells in a 24-well plate with 500 μl of an appropriate culture media, growth factors and agent being tested. Appropriate culture media and growth factors include Advanced DMEM/F12 with media Supplements (1× N2, 1× B27, 2 mM Glutamax, 10 mM HEPES, 1 mM N-acetylcysteine, and 100 U/ml Penicillin/100 μg/ml Streptomycin) and growth factors (50 ng/ml EGF, 50 ng/ml bFGF, and 50 ng/ml IGF-1) as well as the agent(s) being assessed are added into each well. Cells are cultured for 10 days in a standard cell culture incubator at 37° C., and 5% CO2, with media change every 2 days. These cells are then cultured by removing the Stem Cell Proliferation Assay agents and replacing with Basal culture media and molecules to drive differentiation. An appropriate Basal culture media is Advanced DMEM/F12 supplemented with 1× N2, 1× B27, 2 mM Glutamax, 10 mM HEPES, 1 mM N-acetylcysteine, and 100 U/ml Penicillin/100 μg/ml Streptomycin and appropriate molecules to drive differentiation are 3 μM CHIR99021 and 5 μM DAPT for 10 days, with media change every 2 days. The number of hair cells in a population is measured by using flow cytometry for GFP. Hair cell differentiation level can further be assessed using qPCR to measure hair cell marker (e.g. Myo7a) expression level normalized using suitable and unregulated references or housekeeping genes (e.g. Hprt). Hair cell differentiation level can also be assessed by immunostaining for hair cell markers (e.g. Myosin7a, vGlut3, Espin, PMCAs, Ribeye, conjugated-phalloidin, Atoh1, Pou4f3, etc.). Hair cell differentiation level can also be assessed by Western Blot for Myosin7a, vGlut3, Espin, PMCAs, Prestin, Ribeye, Atoh1, Pou4f3.

“Stem Cell Assay” as used herein is an assay in which a cell or a cell population are tested for a series of criteria to determine whether the cell or cell population are stem cells or enriched in stem cells or stem cell markers. In a stem cell assay, the cell/cell population are tested for stem cell characteristics such as expression of Stem Cell Markers, and further optionally are tested for stem cell function, including the capacity of self-renewal and differentiation. Gene expression is measured using methods known in the art such as by PCR, Nanostring, immunostaining, RNAseq, RNA hybridization, or Western blot analysis.

“Stem Cell Proliferation Assay” as used herein is an assay to determine the capacity for agent(s) to induce the creation of stem cells from a starting cell population. In an exemplary Stem Cell Proliferation Assay, the number of cells for an initial cell population is harvested from a Lgr5-GFP mouse such as a B6.129P2-Lgr5tml(cre/ERT2)Cle/J mouse (also known as Lgr5-EGFP-IRES-creERT2 or Lgr5-GFP mouse, Jackson Lab Stock No: 008875) between the age of 0 to 5 days, by isolating the organ of Corti sensory epithelium and dissociating the epithelium into single cells. Approximately 5000 cells are entrapped in 40 μl of culture substrate (for example: Matrigel (Corning, Growth Factor Reduced)) and placed at the center of wells in a 24-well plate with 500 μl of an appropriate culture media, growth factors and agent being tested. Appropriate culture media and growth factors include Advanced DMEN/F 12 with media Supplements (1× N2, 1× B27, 2 mM Glutamax, 10 mM HEPES, 1 mM N-acetylcysteine, and 100 U/ml Penicillin/100 μg/ml Streptomycin) and growth factors (50 ng/ml EGF, 50 ng/ml bFGF, and 50 ng/ml IGF-1) as well as the agent(s) being assessed are added into each well. Cells are cultured for 10 days in a standard cell culture incubator at 37° C. and 5% CO2, with media change every 2 days. The number of Lgr5+ cells is quantified by counting the number of cells identified as Lgr5+ in an In Vitro Lgr5 activity assay. The fraction of cells that are Lgr5+ is quantified by dividing the number of cells identified as Lgr5+ in a cell population by the total number of cells present in the cell population. The number of hair cells in a population is measured by staining with hair cell marker (e.g. MyosinVIIa), or using an endogenous reporter of hair cell genes (e.g. Pou4f3-GFP, Atoh1-nGFP) and analyzing using flow cytometry. The fraction of cells that are hair cells is quantified by dividing the number of cells identified as hair cells in a cell population by the total number of cells present in the cell population. Gene and/or protein expression and/or activity is measured in this assay using methods known in the art such as by PCR, Nanostring, immunostaining, RNAseq, RNA hybridization, or Western blot analysis.

“Stern Cell Markers” as used herein can be defined as gene products (e.g. protein, RNA, etc.) that specifically expressed in stem cells. One type of stem cell marker is gene products that are directly and specifically support the maintenance of stem cell identity. Examples include Lgr5 and Sox2. Additional stem cell markers can be identified using assays that were described in the literatures. To determine whether a gene is required for maintenance of stem cell identity, gain-of-function and loss-of-function studies can be used. In gain-of-function studies, over expression of specific gene product (the stem cell marker) would help maintain the stem cell identity. While in loss-of-function studies, removal of the stem cell marker would cause loss of the stem cell identity or induced the differentiation of stem cells. Another type of stem cell marker is gene that only expressed in stem cells but does not necessary to have specific function to maintain the identity of stem cells. This type of markers can be identified by comparing the gene expression signature of sorted stem cells and non-stem cells by assays such as micro-array and qPCR. This type of stem cell marker can be found in the literature. (e.g. Liu Q. et al., Int J Biochem Cell Biol. 2015 March; 60:99-111 http://www.ncbi.nlm.nih.gov/pubmed/25582750). Potential stem cell markers include Ccdc121, Gdf10, Opcm1, Phex, etc. The expression of stem cell markers such as Lgr5 or Sox2 in a given cell or cell population can be measure using assays such as qPCR, immunohistochemistry, western blot, and RNA hybridization. The expression of stem cell markers can also be measured using transgenic cells express reporters which can indicate the expression of the given stem cell markers, e.g. Lgr5-GFP or Sox2-GFP. Flow cytometry analysis can then be used to measure the activity of reporter expression. Fluorescence microscopy can also be used to directly visualize the expression of reporters. The expression of stem cell markers may further be determined using microarray analysis for global gene expression profile analysis. The gene expression profile of a given cell population or purified cell population can be compared with the gene expression profile of the stem cell to determine similarity between the 2 cell populations. Stern cell function can be measured by colony forming assay or sphere forming assay, self-renewal assay and differentiation assay. In colony (or sphere) forming assay, when cultured in appropriate culture media, the stem cell should be able to form colonies, on cell culture surface (e.g. cell culture dish) or embedded in cell culture substrate (e.g. Matrigel) or he able to form spheres when cultured in suspension. In colony/sphere forming assay, single stem cells are seeded at low cell density in appropriate culture media and allowed to proliferate for a given period of time (7-10 days). Colony formed are then counted and scored for stem cell marker expression as an indicator of sternness of the original cell. Optionally, the colonies that formed are then picked and passaged to test its self-renewal and differentiation potential. In self-renewal assay, when cultured in appropriate culture media, the cells should maintain stem cell marker (e.g. Lgr5) expression over at least one (e.g. 1, 2, 3, 4, 5, 10, 20, etc.) cell divisions. In a Stem Cell Differentiation Assay, when cultured in appropriate differentiation media, the cells should be able to generate hair cell which can be identified by hair cell marker expression measured by qPCR, immunostaining, western blot, RNA hybridization or flow cytometry.

“Subject” includes humans and mammals (e.g. mice, rats, pigs, cats, dogs, and horses). In some embodiments, subjects are be mammals, particularly primates, especially humans. In some embodiments, subjects are livestock such as cattle, sheep, goats, cows, swine, and the like; poultry such as chickens, ducks, geese, turkeys, and the like; and domesticated animals particularly pets such as dogs and cats. In some embodiments (e.g. particularly in research contexts) subject mammals will be, for example, rodents (e.g. mice, rats, hamsters), rabbits, primates, or swine such as inbred pigs and the like.

“Supporting Cell” as used herein in connection with a cochlear epithelium comprises epithelial cells within the organ of Corti that are not hair cells. This includes inner pillar cells, outer pillar cells, inner phalangeal cells, Deiter cells, Hensen cells, Boettcher cells, and/or Claudius cells.

By “statistically significant”, it is meant that the result was unlikely to have occurred by chance. Statistical significance can be determined by any method known in the art. Commonly used measures of significance include the p-value, which is the frequency or probability with which the observed event would occur, if the null hypothesis were true. If the obtained p-value is smaller than the significance level, then the null hypothesis is rejected. In simple cases, the significance level is defined at a p-value of 0.05 or less.

“Substantially” or “essentially” means nearly totally or completely, for instance, 95% or greater of some given quantity.

“Synergist” refers to a compound that causes a more than additive increase in target gene expression or protein levels by 1.1, 1.2, 1.3, 1.4, 1.5, 1.6, 1.7, 1.8, 1.9, 2, 3, 4, 5, 6, 7, 8, 9, 10, 15, 20, 30, 40, 50, 60, 70, 80, 90, 100, 200, 500, 1000-fold more than the additive value of each compound used individually.

“Tissue” is an ensemble of similar cells from the same origin that together carry out a specific function including, for example, tissue of cochlear, such as the organ of Corti.

“Transtympanic” administration refers to direct injection of a composition across the tympanic membrane into the middle ear.

“Treating” as used herein in connection with a cell population means delivering a substance to the population to affect an outcome. In the case of in vitro populations, the substance is directly (or even indirectly) delivered to the population. In the case of in vivo populations, the substance is delivered by administration to the host subject.

“Vehicle Control” or “Control” refers to treatment with the carrier that is absent of drug, such as DMSO for in vitro assays, poloxamer for middle ear delivery, and/or carrier or solution used to deliver drug compounds to cochlear cells describe here.

It is to be appreciated that references to “treating” or “treatment” include the alleviation of established symptoms of a condition. “Treating” or “treatment” of a state, disorder or condition therefore includes: (1) preventing or delaying the appearance of clinical symptoms of the state, disorder or condition developing in a human that is afflicted with or predisposed to the state, disorder or condition but does not yet experience or display clinical or subclinical symptoms of the state, disorder or condition, (2) inhibiting the state, disorder or condition, i.e. arresting, reducing or delaying the development of the disease or a relapse thereof (in case of maintenance treatment) or at least one clinical or subclinical symptom thereof, or (3) relieving or attenuating the disease, i.e, causing regression of the state, disorder or condition or at least one of its clinical or subclinical symptoms.

A “therapeutically effective amount” means the amount of a compound that, when administered to a mammal for treating a disease, is sufficient to effect such treatment for the disease. The “therapeutically effective amount” will vary depending on the compound, the disease and its severity and the age, weight, etc., of the mammal to be treated.

“Wnt activation” as used herein is an activation of the Wnt signaling pathway.

“Wnt alone” as used herein means when the activity as described herein of another agent or combination of agents is compared the activity of “Wnt alone” it is meant comparison is made using the same the Wnt agent at the same concentration.

The term “alkyl” as used herein refers to a straight or branched saturated hydrocarbon. For example, an alkyl group can have 1 to 8 carbon atoms (i.e. (C₁-C₈)alkyl) or 1 to 6 carbon atoms (i.e. (C₁-C₆ alkyl) or 1 to 4 carbon atoms.

The term “alkenyl” as used herein refers to a linear or branched hydrocarbon radical which includes one or more double bonds and can include divalent radicals, having from 2 to about 15 carbon atoms. Examples of alkenyl groups include but are not limited to, ethenyl, propenyl, butenyl, and higher homologs and isomers.

The term “alkynyl” as used herein refers to a linear or branched hydrocarbon radical which includes one or more triple bonds and can include divalent radicals, having from 2 to about 15 carbon atoms. Examples of alkynyl groups include but are not limited to, ethynyl, propynyl, butynyl, and higher homologs and isomers.

The term “halo” or “halogen” as used herein refers to fluoro, chloro, bromo and iodo.

The term “aryl” as used herein refers to a single all carbon aromatic ring or a multiple condensed all carbon ring system wherein at least one of the rings is aromatic. For example, an aryl group can have 6 to 20 carbon atoms, 6 to 14 carbon atoms, or 6 to 12 carbon atoms. Aryl includes a phenyl radical. Aryl also includes multiple condensed ring systems (e.g. ring systems comprising 2, 3 or 4 rings) having about 9 to 20 carbon atoms in which at least one ring is aromatic and wherein the other rings is aromatic or not aromatic (i.e. carbocycle). Such multiple condensed ring systems is optionally substituted with one or more (e.g. 1, 2 or 3) oxo groups on any carbocycle portion of the multiple condensed ring system. The rings of the multiple condensed ring system can be connected to each other via fused, Spiro and bridged bonds when allowed by valency requirements. It is to be understood that the point of attachment of a multiple condensed ring system, as defined above, can be at any position of the ring system including an aromatic or a carbocycle portion of the ring.

The term “heteroaryl” as used herein refers to a single aromatic ring that has at least one atom other than carbon in the ring, wherein the atom is selected from the group consisting of oxygen, nitrogen and sulfur; the term also includes multiple condensed ring systems that have at least one such aromatic ring, which multiple condensed ring systems are further described below. Thus, the term includes single aromatic rings of from about 1 to 6 carbon atoms and about 1-4 heteroatoms selected from the group consisting of oxygen, nitrogen and sulfur in the rings. The sulfur and nitrogen atoms may also be present in an oxidized form provided the ring is aromatic. The term also includes multiple condensed ring systems (e.g. ring systems comprising 2, 3 or 4 rings) wherein a heteroaryl group, as defined above, can be condensed with one or more rings selected from heteroaryls (to form for example a naphthyridinyl such as 1,8-naphthyridinyl), heterocycles, (to form for example a 1,2,3,4-tetrahydronaphthyridinyl such as 1,2,3,4-tetrahydro-1,8-naphthyridinyl), carbocycles (to form for example 5,6,7,8-tetrahydroquinolyl) and aryls (to form for example indazolyl) to form the multiple condensed ring system. Thus, a heteroaryl (a single aromatic ring or multiple condensed ring system) has about 1-20 carbon atoms and about 1-6 heteroatoms within the heteroaryl ring. Such multiple condensed ring systems is optionally substituted with one or more (e.g. 1, 2, 3 or 4) oxo groups on the carbocycle or heterocycle portions of the condensed ring. The rings of the multiple condensed ring system can be connected to each other via fused, Spiro and bridged bonds when allowed by valency requirements. It is to be understood that the individual rings of the multiple condensed ring system is connected in any order relative to one another. It is also to be understood that the point of attachment of a multiple condensed ring system (as defined above for a heteroaryl) can be at any position of the multiple condensed ring system including a heteroaryl, heterocycle, aryl or carbocycle portion of the multiple condensed ring system and at any suitable atom of the multiple condensed ring system including a carbon atom and heteroatom (e.g, a nitrogen).

The term “cycloalkyl” as used herein refers to a saturated or partially saturated ring structure having about 3 to about 8 ring members that has only carbon atoms as ring atoms and can include divalent radicals. Examples of cycloalkyl groups include but are not limited to, cyclopropyl, cyclobutyl, cyclopentyl, cyclohexyl, cyclohexene, cyclopentenyl, cyclohexenyl.

The terms “heterocyclyl” or “heterocyclic” refer to monocyclic or polycyclic 3 to 24-membered rings containing carbon and heteroatoms selected from oxygen, phosphorous, nitrogen, or sulfur and wherein there are no delocalized π electrons (aromaticity) shared among the ring carbon or heteroatoms. Examples of heterocyclyl rings include, but are not limited to, oxetanyl, azetadinyl, tetrahydrofuranyl, pyrrolidinyl, oxazolinyl, oxazolidinyl, thiazolinyl, thiazolidinyl, pyranyl, thiopyranyl, tetrahydropyranyl, dioxalinyl, piperidinyl, morpholinyl, thiomorpholinyl, thiomorpholinyl S-oxide, thiomorpholinyl S-dioxide, piperazinyl, azepinyl, oxepinyl, diazepinyl, tropanyl, and homotropanyl. A heterocyclyl or heterocycloalkyl ring can also be fused or bridged, e.g. can be a bicyclic ring. Examples of heterocyclyl also include, but are not limited to, fused rings, bridged rings (e.g. 2,5-diazabicyclo[1,2,1]heptane), and spirocyclic rings, (e.g. 2,8-diazaspiro[4,5]decane).

As used herein, “alkyl”, “C₁, C₂, C₃, C₄, C₅ or C₆ alkyl” or “C₁-C₆ alkyl” is intended to include C₁, C₂, C₃, C₄, C₅ or C₆ straight chain (linear) saturated aliphatic hydrocarbon groups and C₃, C₄, C₅ or C₆ branched saturated aliphatic hydrocarbon groups. For example, C₁-C₆ alkyl is intends to include C₁, C₂, C₃, C₄, C₅ and C₆ alkyl groups. Examples of alkyl include, moieties having from one to six carbon atoms, such as, but not limited to, methyl, ethyl, n-propyl, i-propyl, n-butyl, s-butyl, t-butyl, n-pentyl, i-pentyl or n-hexyl. In some embodiments, a straight chain or branched alkyl has six or fewer carbon atoms (e.g. C₁-C₆ for straight chain, C₃-C₆ for branched chain), and in another embodiment, a straight chain or branched alkyl has four or fewer carbon atoms.

As used herein, the term “optionally substituted alkyl” refers to unsubstituted alkyl or alkyl having designated substituents replacing one or more hydrogen atoms on one or more carbons of the hydrocarbon backbone. Such substituents can include, for example, alkyl, alkenyl, alkynyl, halogen, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, amino (including alkylamino, dialkylamino, arylamino, diarylamino and alkylarylamino), acylamino (including alkylcarbonylamino, arylcarbonylamino, carbamoyl and ureido), amidino, imino, sulphhydryl, alkylthio, arylthio, thiocarboxylate, sulphates, alkylsulphinyl, sulphonato, sulphamoyl, sulphonamido, nitro, trifluoromethyl, cyano, azido, heterocyclyl, alkylaryl, or an aromatic or heteroaromatic moiety.

As used herein, the term “alkenyl” includes unsaturated aliphatic groups analogous in length and possible substitution to the alkyls described above, but that contain at least one double bond. For example, the term “alkenyl” includes straight chain alkenyl groups (e.g. ethenyl, propenyl, butenyl, pentenyl, hexenyl, heptenyl, octenyl, nonenyl, decenyl), and branched alkenyl groups. In certain embodiments, a straight chain or branched alkenyl group has six or fewer carbon atoms in its backbone (e.g. C₂-C₆ for straight chain, C₃-C₆ for branched chain). The term “C₂-C₆” includes alkenyl groups containing two to six carbon atoms. The term “C₃-C₆” includes alkenyl groups containing three to six carbon atoms.

As used herein, the term “optionally substituted alkenyl” refers to unsubstituted alkenyl or alkenyl having designated substituents replacing one or more hydrogen atoms on one or more hydrocarbon backbone carbon atoms. Such substituents can include, for example, alkyl, alkenyl, alkynyl, halogen, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, amino (including alkylamino, dialkylamino, arylamino, diarylamino and alkylarylamino), acylamino (including alkylcarbonylamino, arylcarbonylamino, carbamoyl and ureido), amidino, imino, sulphhydryl, alkylthio, arylthio, thiocarboxylate, sulphates, alkylsulphinyl, sulphonato, sulphamoyl, sulphonamido, nitro, trifluoromethyl, cyano, heterocyclyl, alkylaryl, or an aromatic or heteroaromatic moiety.

As used herein, the term “alkynyl” includes unsaturated aliphatic groups analogous in length and possible substitution to the alkyls described above, but which contain at least one triple bond. For example, “alkynyl” includes straight chain alkynyl groups (e.g. ethynyl, propynyl, butynyl, pentynyl, hexynyl, heptynyl, octynyl, nonynyl, decynyl), and branched alkynyl groups. In certain embodiments, a straight chain or branched alkynyl group has six or fewer carbon atoms in its backbone (e.g. C₂-C₆ for straight chain, C₃-C₆ for branched chain). The term “C₂-C₆” includes alkynyl groups containing two to six carbon atoms. The term “C₃-C₆” includes alkynyl groups containing three to six carbon atoms. As used herein, “C₂-C₆ alkenylene linker” or “C₂-C₆ alkynylene linker” is intended to include C₂, C₃, C₄, C₅ or C₆ chain (linear or branched) divalent unsaturated aliphatic hydrocarbon groups. For example, C₂-C₆ alkenylene linker is intended to include C₂, C₃, C₄, C₅ and C₆ alkenylene linker groups.

As used herein, the term “optionally substituted alkynyl” refers to unsubstituted alkynyl or alkynyl having designated substituents replacing one or more hydrogen atoms on one or more hydrocarbon backbone carbon atoms. Such substituents can include, for example, alkyl, alkenyl, alkynyl, halogen, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, amino (including alkylamino, dialkylamino, arylamino, diarylamino and alkylarylamino), acylamino (including alkylcarbonylamino, arylcarbonylamino, carbamoyl and ureido), amidino, imino, sulphhydryl, alkylthio, arylthio, thiocarboxylate, sulphates, alkylsulphinyl, sulphonato, sulphamoyl, sulphonamido, nitro, trifluoromethyl, cyano, azido, heterocyclyl, alkylaryl, or an aromatic or heteroaromatic moiety.

Other optionally substituted moieties (such as optionally substituted cycloalkyl, heterocycloalkyl, aryl, or heteroaryl) include both the unsubstituted moieties and the moieties having one or more of the designated substituents. For example, substituted heterocycloalkyl includes those substituted with one or more alkyl groups, such as 2,2,6,6-tetramethyl-piperidinyl and 2,2,6,6-tetramethyl-1,2,3,6-tetrahydropyridinyl.

As used herein, the term “cycloalkyl” refers to a saturated or partially unsaturated hydrocarbon monocyclic or polycyclic (e.g. fused, bridged, or spiro rings) system having 3 to 30 carbon atoms (e.g. C₃-C₁₂, C₃-C₁₀, or C₃-C₈). Examples of cycloalkyl include, but are not limited to, cyclopropyl, cyclobutyl, cyclopentyl, cyclohexyl, cycloheptyl, cyclooctyl, cyclopentenyl, cyclohexenyl, cycloheptenyl, 1,2,3,4-tetrahydronaphthalenyl, and adamantyl. In the case of polycyclic cycloalkyl, only one of the rings in the cycloalkyl needs to be non-aromatic. In some embodiments, the cycloalkyl is hexahydroindacenyl. In some embodiments, the cycloalkyl is

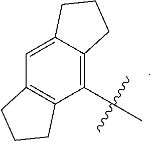 [SMILES: *c1c2c(cc3c1CCC3)CCC2]

As used herein, the term “heterocycloalkyl” refers to a saturated or partially unsaturated 3-8 membered monocyclic, 7-12 membered bicyclic (fused, bridged, or Spiro rings), or 11-14 membered tricyclic ring system (fused, bridged, or spiro rings) having one or more heteroatoms (such as O, N, S, P, or Se), e.g. 1 or 1-2 or 1-3 or 1-4 or 1-5 or 1-6 heteroatoms, or e.g. , 1, 2, 3, 4, 5, or 6 heteroatoms, independently selected from the group consisting of nitrogen, oxygen and sulphur, unless specified otherwise. Examples of heterocycloalkyl groups include, but are not limited to, piperidinyl, piperazinyl, pyrrolidinyl, dioxanyl, tetrahydrofuranyl, isoindolinyl, indolinyl, imidazolidinyl, pyrazolidinyl, oxazolidinyl, isoxazolidinyl, triazolidinyl, oxiranyl, azetidinyl, oxetanyl, thietanyl, 1,2,3,6-tetrahydropyridinyl, tetrahydropyranyl, dihydropyranyl, pyranyl, morpholinyl, tetrahydrothiopyranyl, 1,4-diazepanyl, 1,4-oxazepanyl, 2-oxa-5-azabicyclo[2.2.1]heptanyl, 2,5-diazabicyclo[2.2.1]heptanyl, 2-oxa-6-azaspiro[3.3]heptanyl, 2,6-diazaspiro[3.3]heptanyl, 1,4-dioxa-8-azaspiro[4.5]decanyl, 1,4-dioxaspiro[4.5]decanyl, 1-oxaspiro[4.5]decanyl, 1-azaspiro[4.5]decanyl, 3′H-spiro[cyclohexane-1,1′-isobenzofuran]-yl, 7′H-spiro[cyclohexane-1,5′-furo[3,4-b]pyridin]-yl 3′H-spiro[cyclohexane-1,1′-furo[3,4-c]pyridin]-yl, 3-azabicyclo[3.1.0]hexanyl, 3-azabicyclo[3.1.0]hexan-3-yl, 1,4,5,6-tetrahydropyrrolo[3,4-c]pyrazolyl, 3,4,5,6,7,8-hexahydropyrido[4,3-d]pyrimidinyl, 4,5,6,7-tetrahydro-1H-pyrazolo[3,4-c]pyridinyl, 5,6,7,8-tetrahydropyrido[4,3-d]pyrimidinyl, 2-azaspiro[3.3]heptanyl, 2-methyl-2-azaspiro[3.3]heptanyl, 2-azaspiro[3.5]nonanyl, 2-methyl-2-azaspiro[3.5]nonanyl, 2-azaspiro[4.5]decanyl, 2-methyl-2-azaspiro[4.5]decanyl, 2-oxa-azaspiro[3.4]octanyl, 2-oxa-azaspiro[3.4]octan-6-yl, and the like. In the case of multicyclic heterocycloalkyl, only one of the rings in the heterocycloalkyl needs to be non-aromatic (e.g. 4,5,6,7-tetrahydrobenzo[c]isoxazolyl).

As used herein, the term “aryl” includes groups with aromaticity, including “conjugated,” or multicyclic systems with one or more aromatic rings and do not contain any heteroatom in the ring structure. The term aryl includes both monovalent species and divalent species. Examples of aryl groups include, but are not limited to, phenyl, biphenyl, naphthyl and the like. Conveniently, an aryl is phenyl.

As used herein, the term “heteroaryl” is intended to include a stable 5-, 6-, or 7-membered monocyclic or 7-, 8-, 9-, 10-, 11- or 12-membered bicyclic aromatic heterocyclic ring which consists of carbon atoms and one or more heteroatoms. e.g. 1 or 1-2 or 1-3 or 1-4 or 1-5 or 1-6 heteroatoms, or e.g., 1, 2, 3, 4, 5, or 6 heteroatoms, independently selected from the group consisting of nitrogen, oxygen and sulphur. The nitrogen atom is substituted or unsubstituted (i.e. N or NR wherein R is H or other substituents, as defined). The nitrogen and sulphur heteroatoms may optionally be oxidised (i.e. N→O and S(O)ₚ, where p=1 or 2). It is to be noted that total number of S and O atoms in the aromatic heterocycle is not more than 1. Examples of heteroaryl groups include pyrrole, furan, thiophene, thiazole, isothiazole, imidazole, triazole, tetrazole, pyrazole, oxazole, isoxazole, pyridine, pyrazine, pyridazine, pyrimidine, and the like. Heteroaryl groups can also be fused or bridged with alicyclic or heterocyclic rings, which are not aromatic so as to form a multicyclic system 4,5,6,7-tetrahydrobenzo[c]isoxazolyl).

Furthermore, the terms “aryl” and “heteroaryl” include multicyclic aryl and heteroaryl groups, e.g, tricyclic, bicyclic, e.g. naphthalene, benzoxazole, benzodioxazole, benzothiazole, benzoimidazole, benzothiophene, quinoline, isoquinoline, naphthrydine, indole, benzofuran, purine, benzofuran, deazapurine, indolizine.

The cycloalkyl, heterocycloalkyl, aryl, or heteroaryl ring can be substituted at one or more ring positions (e.g. the ring-forming carbon or heteroatom such as N) with such substituents as described above, for example, alkyl, alkenyl, alkynyl, halogen, hydroxyl, alkoxy, alkylcarbonyloxy, arylcarbonyloxy, alkoxycarbonyloxy, aryloxycarbonyloxy, carboxylate, alkylcarbonyl, alkylaminocarbonyl, aralkylaminocarbonyl, alkenylaminocarbonyl, alkylcarbonyl, arylcarbonyl, aralkylcarbonyl, alkenylcarbonyl, alkoxycarbonyl, aminocarbonyl, alkylthiocarbonyl, phosphate, phosphonato, phosphinato, amino (including alkylamino, dialkylamino, arylamino, diarylamino and alkylarylamino), acylamino (including alkylcarbonylamino, arylcarbonylamino, carbamoyl and ureido), amidino, imino, sulphhydryl, alkylthio, arylthio, thiocarboxylate, sulphates, alkylsulphinyl, sulphonato, sulphamoyl, sulphonamido, nitro, trifluoromethyl, cyano, azido, heterocyclyl, alkylaryl, or an aromatic or heteroaromatic moiety. Aryl and heteroaryl groups can also be fused or bridged with alicyclic or heterocyclic rings, which are not aromatic so as to form a multicyclic system (e.g. tetralin, methylenedioxyphenyl such as benzo[d][1,3]dioxole-5-yl).

As used herein, the term “substituted,” means that any one or more hydrogen atoms on the designated atom is replaced with a selection from the indicated groups, provided that the designated atom's normal valency is not exceeded, and that the substitution results in a stable compound. When a substituent is oxo or keto (i.e. ═O), then 2 hydrogen atoms on the atom are replaced. Keto substituents are not present on aromatic moieties. Ring double bonds, as used herein, are double bonds that are formed between two adjacent ring atoms (e.g. C═C, C═N or N═N). “Stable compound” and “stable structure” are meant to indicate a compound that is sufficiently robust to survive isolation to a useful degree of purity from a reaction mixture, and formulation into an efficacious therapeutic agent.

When a bond to a substituent is shown to cross a bond connecting two atoms in a ring, then such substituent is bonded to any atom in the ring. When a substituent is listed without indicating the atom via which such substituent is bonded to the rest of the compound of a given formula, then such substituent is bonded via any atom in such formula. Combinations of substituents and/or variables are permissible, but only if such combinations result in stable compounds.

When any variable (e.g. R) occurs more than one time in any constituent or formula for a compound, its definition at each occurrence is independent of its definition at every other occurrence. Thus, for example, if a group is shown to be substituted with 0-2 R moieties, then the group may optionally be substituted with up to two R moieties and R at each occurrence is selected independently from the definition of R. Also, combinations of substituents and/or variables are permissible, but only if such combinations result in stable compounds.

As used herein, the term “hydroxy” or “hydroxyl” includes groups with an —OH or —O⁻.

As used herein, the term “halo” or “halogen” refers to fluoro, chloro, bromo and iodo.

The term “haloalkyl” or “haloalkoxyl” refers to an alkyl or alkoxyl substituted with one or more halogen atoms.

As used herein, the term “optionally substituted haloalkyl” refers to unsubstituted haloalkyl having designated substituents replacing one or more hydrogen atoms on one or more hydrocarbon backbone carbon atoms. Such substituents can include, for example, alkyl, alkenyl, alkynyl, halogen, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, amino (including alkylamino, dialkylamino, arylamino, diarylamino and alkylarylamino), acylamino (including alkylcarbonylamino arylcarbonylamino, carbamoyl and ureido), amidino, imino, sulphhydryl, alkylthio, arylthio, thiocarboxylate, sulphates, alkylsulphinyl, sulphonato, sulphamoyl, sulphonamido, nitro, trifluoromethyl, cyano, azido, heterocyclyl, alkylaryl, or an aromatic or heteroaromatic moiety.

As used herein, the term “alkoxy” or “alkoxyl” includes substituted and unsubstituted alkyl, alkenyl and alkynyl groups covalently linked to an oxygen atom. Examples of alkoxy groups or alkoxyl radicals include, but are not limited to, methoxy, ethoxy, isopropyloxy, propoxy, butoxy and pentoxy groups. Examples of substituted alkoxy groups include halogenated alkoxy groups. The alkoxy groups can be substituted with groups such as alkenyl, alkynyl, halogen, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, amino (including alkylamino, dialkylamino, arylamino, diarylamino, and alkylarylamino), acylamino (including alkylcarbonylamino, arylcarbonylamino, carbamoyl and ureido), amidino, imino, sulphhydryl, alkylthio, arylthio, thiocarboxylate, sulphates, alkylsulphinyl, sulphonato, sulphamoyl, sulphonamido, nitro, trifluoromethyl, cyano, azido, heterocyclyl, alkylaryl, or an aromatic or heteroaromatic moieties. Examples of halogen substituted alkoxy groups include, but are not limited to, fluoromethoxy, difluoromethoxy, trifluoromethoxy, chloromethoxy, dichloromethoxy and trichloromethoxy.

### Enumerated Embodiments

### LSD1 Inhibitor+Wnt Agonist Embodiments

Embodiment 1. A method for increasing proliferation of a cochlear supporting cell or a vestibular supporting cell, comprising contacting the supporting cell with:
   a) a first epigenetic agent that is a lysine specific demethylase 1 (LSD1) inhibitor; and
   b) a Wnt agonist;
   wherein steps (a) and (b) can occur in any order or simultaneously, thereby increasing cochlear supporting cell or vestibular supporting cell proliferation compared to a vehicle control.
Embodiment 2. A method for producing an expanded population of cochlear or vestibular cells, comprising contacting a population of cochlear supporting cells or vestibular supporting cells with:
   a) a first epigenetic agent that is a lysine specific demethylase 1 (LSD1) inhibitor and;
   b) a Wnt agonist
   wherein steps (a) and (b) can occur in any order or simultaneously, thereby producing an expanded population of cochlear or vestibular cells compared to a vehicle control.
Embodiment 3. The method of Embodiment 1 or Embodiment 2, wherein the cochlear supporting cell(s) or vestibular supporting cell(s) express(es) leucine-rich repeat-containing G-protein coupled receptor 5 (Lgr5).
Embodiment 4. The method of any preceding Embodiment, wherein the cochlear supporting cell(s) or vestibular supporting cell(s) are/is a mature cell(s).
Embodiment 5. The method of any of Embodiments 2-4, wherein the expanded population of cochlear or vestibular cells expresses leucine-rich repeat-containing G-protein coupled receptor 5 (Lgr5).
Embodiment 6. The method of any preceding Embodiment, wherein the cochlear supporting cell(s) or vestibular supporting cell(s) are/is a cochlear supporting cell(s).
Embodiment 7. The method of Embodiment 6, wherein the expanded population of cochlear or vestibular cells are cochlear cells.
Embodiment 8. The method of any preceding Embodiment, wherein the LSD1 inhibitor in combination with the Wnt agonist increases the Lgr5 Activity of the expanded population of cochlear or vestibular cells by a factor of at least 10, 20, 30, 40, 50, 75, 100 or 200% compared to a Wnt agonist alone or a Wnt agonist in combination with valproic acid, wherein the Lgr5 Activity is measured in a Stem Cell Proliferation Assay
Embodiment 9. A method of treating a subject who has, or is at risk of, developing an inner ear hearing or balance disorder, comprising administering to the subject:
   a) a first epigenetic agent that is a lysine specific demethylase 1 (LSD1) inhibitor; and
   b) a Wnt agonist
   wherein step (a) and (b) can occur in any order or simultaneously.
Embodiment 10. The method of Embodiment 9, wherein the subject has an inner ear hearing or balance disorder.
Embodiment 11. The method of Embodiment 9 or 10, wherein the disorder is an inner ear hearing disorder.
Embodiment 12. The method of Embodiment 9 or 10, wherein the disorder is a balance disorder
Embodiment 13. The method of any of Embodiments 9-12, wherein the inner ear hearing or balance disorder is sensorineural hearing loss.
Embodiment 14. The method of any of Embodiments 9-13, wherein the treatment results in improved auditory function when assessed by behavioural audiometry or auditory brainstem response (ABR) testing.
Embodiment 15. The method of any preceding Embodiment, wherein the LSD1 inhibitor is irreversible.
Embodiment 16. The method of any preceding Embodiments, wherein the LSD1 inhibitor is selected from the group consisting of GSK-2879552, GSK-LSD1, Tranylcypromine, ORY-1001, RN-1, and Phenelzine sulfate.
Embodiment 17. The method of any preceding Embodiments, wherein the LSD1 inhibitor is GSK2879552.
Embodiment 18. The method of any preceding Embodiments, wherein the LSD1 inhibitor is GSK-LSD1.
Embodiment 19. The method of any preceding Embodiments, wherein the LSD1 inhibitor is Tranylcypromine.
Embodiment 20. The method of any preceding Embodiments, wherein the LSD1 inhibitor is Phenelzine sulfate.
Embodiment 21. The method of Embodiments 17, wherein GSK2879552 is at a concentration of about between 4 nM to 30 μM.
Embodiment 22. The method of Embodiments 18, wherein GSK-LSD1 is at a concentration of about between 4 nM to 50 μM.
Embodiment 23. The method of Embodiments 19, wherein Tranylcypromine is at a concentration of about between 0.1 μM to 20 μM.
Embodiment 24. The method of Embodiments 20, wherein Phenelzine sulfate at a concentration of about between 0.1 μM to 10 μM.
Embodiment 25. The method of any preceding Embodiments, wherein the Wnt agonist is a GSK3 inhibitor.
Embodiment The method of Embodiments 25, wherein the GSK3 inhibitor is selected from the group consisting of: AZD1080, LY2090314, a substituted 3-Imidazo[1,2-a]pyridin-3-yl-4-(1,2,3,4-tetrahydro-[1,4]diazepino-[6,7,1-hi]indol-7-yl)pyrrole-2,5-dione, GSK3 inhibitor XXII or CHIR99021.
Embodiment 27. The method of Embodiments 26, wherein the GSK3 inhibitor is AZD1080.
Embodiment 28. The method of Embodiments 26, wherein the GSK3 inhibitor is LY2090314.
Embodiment 29. The method of Embodiments 26, wherein the GSK3 inhibitor is a substituted 3-Imidazo[1,2-a]pyridin-3-yl-4-(1,2,3,4-tetrahydro-[1,4]diazepino-[6,7,1-hi]indol-7-yl)pyrrole-2,5-dione.
Embodiment 30. The method of Embodiments 26, wherein the GSK3 inhibitor is GSK3 inhibitor XXII.
Embodiment 31. The method of Embodiments 26, wherein the GSK3 inhibitor is CHIR99021.
Embodiment 32. The method of Embodiments 27, wherein AZD1080 is at a concentration of about between 0.5 μM to 5 μM.
Embodiment 33. The method of Embodiments 28, wherein LY2090314 is at a concentration of about between 4 nM to 40 nM.
Embodiment 34. The method of Embodiments 29, wherein a substituted 3-Imidazo[1,2-a]pyridin-3-yl-4-(1,2,3,4-tetrahydro-[1,4]diazepino-[6,7,1-hi]indol-7-yl)pyrrole-2,5-dione is at a concentration of about between 5 nM to 500 nM.
Embodiment 35. The method of Embodiments 30, wherein GSK3 inhibitor XXII at a concentration of about between 0.1 μM to 1 μM.
Embodiment 36. The method of Embodiments 31, wherein CHIR99021 is at a concentration of about between 1 μM to 10 μM.
Embodiment 37. The method of any preceding Embodiments, further comprising contacting the cochlear or vestibular supporting cell(s) with, or administering to the subject, a second epigenetic agent.
Embodiment 38. The method of any preceding Embodiments, wherein the second epigenetic agent is an HDAC inhibitor, an EZH2 inhibitor, a DOT1L inhibitor, or a KDM inhibitor.
Embodiment 39. The method of Embodiments 38, wherein the HDAC inhibitor is Valproic Acid (VPA)
Embodiment 40. The method of Embodiments 39, wherein the VPA is at a concentration of about between 100 μM to 4,000 μM.
Embodiment 41. The method of Embodiments 38, wherein the EZH2 inhibitor is an enzymatic inhibitor.
Embodiment 42. The method of Embodiments 38, wherein the EZH2 inhibitor is selected from the group consisting of: CPI-1205, CPI-169, E11, PF-06821497, tazemetostat, valemetostat, CPI-360, EPZ011989, UNC 2399, and PF 06726304.
Embodiment 43. The method of Embodiments 42, wherein the EZH2 inhibitor is CPI-1205.
Embodiment 44. The method of Embodiments 42, wherein the EZH2 inhibitor is E11.
Embodiment 45. The method of Embodiments 42, wherein the EZH2 inhibitor is PF-06821497.
Embodiment 46. The method of Embodiments 42, wherein the EZH2 inhibitor is tazemetostat.
Embodiment 47. The method of Embodiments 42, wherein the EZH2 inhibitor is valemetostat.
Embodiment The method of Embodiments 42, wherein the CPI-1205 is at a concentration of about between 10 nM to 1000 nM.
Embodiment 49. The method of Embodiments 44, wherein the E11 is at a concentration of about between 1 μM to 10 μM.
Embodiment 50. The method of Embodiments 45, wherein the PF-06821497 is at a concentration of about between 1 nM to 100 nM.
Embodiment 51. The method of Embodiments 46, wherein the tazemetostat is at a concentration of about between 0.1 μM to 1.5 μM.
Embodiment 52. The method of Embodiments 47, wherein the valemetostat is at a concentration of about between 10 nM to 1000 nM.
Embodiment 53. The method of Embodiments 38, wherein the DOT1L inhibitor is an S-adenosyl methionine (SAM) competitive inhibitor.
Embodiment 54. The method of Embodiments 38, wherein the DOT1L inhibitor is selected from the group consisting of EPZ004777, pinometostat and SGC0946.
Embodiment 55. The method of Embodiments 54, wherein the DOT1L inhibitor is EPZ004777.
Embodiment 56. The method of Embodiments 54, wherein the DOT1L inhibitor is pinometostat.
Embodiment 57. The method of Embodiments 54, wherein the DOT1L inhibitor is SGC0946.
Embodiment 58. The method of Embodiments 55, wherein the EPZ004777 is at a concentration of about between 0.5 μM to 45 μM.
Embodiment 59. The method of Embodiments 56, wherein the pinometostat is at a concentration of about between 0.1 μM to 10 μM.
Embodiment 60. The method of Embodiments 57, wherein the SGC0946 is at a concentration of about between 0.5 μM to 5 μM.
Embodiment 61. The method of Embodiments 38, wherein the KDM inhibitor is AS 8351, EPT 103182, TC-E 5002.
Embodiment 62. The method of Embodiments 61, wherein the KDM inhibitor is AS 8351.
Embodiment 63. The method of Embodiments 61, wherein the KDM inhibitor is TC-E 5002.
Embodiment 64. The method of Embodiments 62, wherein the AS 8351 is at a concentration of about between 0.5 μM to 5 μM.
Embodiment 65. The method of Embodiments 63, wherein the TC-E 5002 is at a concentration of about between 0.1 μM to 10 μM.
Embodiment 72. The method of 69, wherein the TT10 is at a concentration of about between 1 μM to 100 μM.
Embodiment 73. The method of any preceding Embodiments, wherein the LSD1 inhibitor is administered locally and/or systemically.
Embodiment 74. The method of any preceding Embodiments, wherein the LSD1 inhibitor is administered locally.
Embodiment 75. The method of any preceding Embodiments, wherein the LSD1 inhibitor is administered systemically.
Embodiment 76. The method of any preceding Embodiments, wherein the LSD1 inhibitor is administered locally and systemically.
Embodiment 77. The method of any preceding Embodiments, wherein the Wnt agonist is administered locally and/or systemically.
Embodiment 78. The method of any preceding Embodiments, wherein the Wnt agonist is administered locally.
Embodiment 79. The method of any preceding Embodiments, wherein the Wnt agonist is administered systemically.
Embodiment 80. The method of any preceding Embodiments, wherein the Wnt agonist is administered locally and systemically.
Embodiment 81 The method of any preceding Embodiments, wherein the second epigenetic agent is administered locally and/or systemically.
Embodiment 82. The method of any preceding Embodiments, wherein the second epigenetic agent is administered locally.
Embodiment 83. The method of any preceding Embodiments, wherein the second epigenetic agent is administered systemically.
Embodiment 84. The method of any preceding Embodiments, wherein the second epigenetic agent is administered locally and systemically.
Embodiment 85. The method of any of Embodiments 73-84, wherein the local administration is to the tympanic membrane, the middle ear or the inner ear.
Embodiment 86. The method of Embodiments 85, wherein the local administration is to the middle ear
Embodiment 87. The method of any of Embodiments 73-84, wherein the systemic administration is oral or parenteral.
Embodiment 88. The method of Embodiments 87, wherein the systemic administration is oral.
Embodiment 89. The method of any of Embodiments 73-88, wherein the LSD1 inhibitor is GSK2879552 and is administered locally at a dose of 4 nM.
Embodiment 90. The method of any of Embodiments 73-88, wherein the LSD1 inhibitor is tranylcypromine and is administered locally at a dose of 4 μM.
Embodiment 91. The method of any of Embodiments 73-88 wherein the LSD1 inhibitor is GSK2879552 and is administered systemically at a unit dose of 1 mg.
Embodiment 92. The method of any of Embodiments 73-89, wherein the LSD1 inhibitor is tranylcypromine and is administered systemically at a unit dose of 15 mg.
Embodiment 93. The method of any of Embodiments 73-92, wherein the Wnt agonist is CHIR99021 and is administered locally at a dose of 4 μM.
Embodiment 94. The method of any of Embodiments 73-93, wherein the second epigenetic agent is valproic acid (VPA) and is administered locally at a dose of 1 mM
Embodiment 95. The method of any of Embodiments 73-93, wherein the second epigenetic agent is valproic acid (VPA) and is administered systemically at a unit dose of 500 mg.
Embodiment 96. A pharmaceutical composition comprising a first epigenetic agent that is a LSD1 inhibitor, a Wnt agonist and a pharmaceutically acceptable carrier.
Embodiment 97. The pharmaceutical composition of Embodiments 96, wherein the LSD1 inhibitor is irreversible.
Embodiment 98. The pharmaceutical composition of Embodiments 96, wherein the LSD1 inhibitor is selected from the group consisting of GSK-2879552, GSK-LSD1, Tranylcypromine, ORY-1001, RN-1, and Phenelzine sulfate.
Embodiment 99. The pharmaceutical composition of Embodiments 97, wherein the LSD1 inhibitor is GSK2879552.
Embodiment 100. The pharmaceutical composition of Embodiments 97, wherein the LSD1 inhibitor is GSK-LSD1.
Embodiment 101. The pharmaceutical composition Embodiments 97, wherein the LSD1 inhibitor is Tranylcypromine.
Embodiment 102. The pharmaceutical composition of Embodiments 97, wherein the LSD1 inhibitor is Phenelzine sulfate.
Embodiment 103. The pharmaceutical composition of Embodiments 99, wherein GSK2879552 is at a concentration of about between 4 μM to 30 mM.
Embodiment 104. The pharmaceutical composition of Embodiments 100, wherein GSK-LSD1 is at a concentration of about between 4 μM to 50 mM.
Embodiment 105. The pharmaceutical composition of Embodiments 101, wherein Tranylcypromine is at a concentration of about between 0.1 mM to 20 mM.
Embodiment 106. The pharmaceutical composition of Embodiments 102, wherein Phenelzine sulfate is at a concentration of about between 0.1 mM to 10 mM.
Embodiment 107. The pharmaceutical composition of any of Embodiments 96-106, wherein the Wnt agonist is a GSK3 inhibitor.
Embodiment 108. The pharmaceutical composition of Embodiments 107, wherein the GSK3 inhibitor is selected from the group consisting of: AZD1080, LY2090314, a substituted 3-Imidazo[1,2-a]pyridin-3-yl-4-(1,2,3,4-tetrahydro-[1,4]diazepino-[6,7,1-hi]indol-7-yl)pyrrole-2,5-dione, GSK3 inhibitor XXII or CHIR99021.
Embodiment 109. The pharmaceutical composition of Embodiments 108, wherein the GSK3 inhibitor is AZD1080.
Embodiment 110. The pharmaceutical composition of Embodiments 108, wherein the GSK3 inhibitor is LY2090314.
Embodiment 111. The pharmaceutical composition of Embodiments 108, wherein the GSK3 inhibitor is a substituted 3-Imidazo[1,2-a]pyridin-3-yl-4-(1,2,3,4-tetrahydro-[1,4]diazepino-[6,7,1-hi]indol-7-yl)pyrrole-2,5-dione.
Embodiment 112. The pharmaceutical composition of Embodiments 108, wherein the GSK3 inhibitor is GSK3 inhibitor XXII.
Embodiment 113. The pharmaceutical composition of Embodiments 108, wherein the GSK3 inhibitor is CHIR99021.
Embodiment 114. The pharmaceutical composition of Embodiments 109, wherein AZD1080 is at a concentration of about between 0.5 mM to 5 mM.
Embodiment 115. The pharmaceutical composition of Embodiments, 110, wherein LY2090314 is at a concentration of about between 4 μM to 40 μM.
Embodiment 116. The pharmaceutical composition of Embodiments, 111, wherein a substituted 3-Imidazo[1,2-a]pyridin-3-yl-4-(1,2,3,4-tetrahydro-[1,4]diazepino-[6,7,1-hi]indol-7-yl)pyrrole-2,5-dione is at a concentration of about between 5 μM to 500 μM.
Embodiment 117. The pharmaceutical composition of Embodiments, 112 wherein GSK3 inhibitor XXII at a concentration of about between 0.1 mM to 1 mM.
Embodiment 118. The pharmaceutical composition of Embodiments 113, wherein CHIR99021 is at a concentration of about between 1 mM to 10 mM.
Embodiment 119. The pharmaceutical composition of any of Embodiments 96-118 further comprising a second epigenetic agent.
Embodiment 120. The pharmaceutical composition of Embodiments 119, wherein the second epigenetic agent is an HDAC inhibitor, an EZH2 inhibitor, a DOT1L inhibitor, or a KDM inhibitor.
Embodiment 121. The pharmaceutical composition of Embodiments 120, wherein the HDAC inhibitor is Valproic Acid (VPA)
Embodiment 122. The pharmaceutical composition of Embodiments 121, wherein the VPA is at a concentration of about between 100 mM to 4,000 mM.
Embodiment 123. The pharmaceutical composition of Embodiments 120, wherein the EZH2 inhibitor is an enzymatic inhibitor.
Embodiment 124. The pharmaceutical composition of Embodiments 120, wherein the EZH2 inhibitor is selected from the group consisting of: CPI-1205, CPI-169, E11, PF-06821497, tazemetostat, valemetostat CPI-360, EPZ011989, UNC 2399, and PF 06726304.
Embodiment 125. The pharmaceutical composition of Embodiments 124, wherein the EZH2 inhibitor is CPI-1205.
Embodiment 126. The pharmaceutical composition of Embodiments 124, wherein the EZH2 inhibitor is E11.
Embodiment 127. The pharmaceutical composition of Embodiments 124, wherein the EZH2 inhibitor is PF-06821497.
Embodiment 128. The pharmaceutical composition of Embodiments 124, wherein the EZH2 inhibitor is tazemetostat.
Embodiment 129. The pharmaceutical composition of Embodiments 124, wherein the EZH2 inhibitor is valemetostat.
Embodiment 130. The pharmaceutical composition of Embodiments 125, wherein the CPI-1205 is at a concentration of about between 10 μM to 1000 μM.
Embodiment 131. The pharmaceutical composition of Embodiments 126, wherein the E11 is at a concentration of about between 1 mM to 10 mM.
Embodiment 132. The pharmaceutical composition of Embodiments 127, wherein the PF-06821497 is at a concentration of about between 1 μM to 100 μM.
Embodiment 133. The pharmaceutical composition of Embodiments 128, wherein the tazemetostat is at a concentration of about between 0.1 mM to 10 mM.
Embodiment 134. The pharmaceutical composition of Embodiments 129, wherein the valemetostat is at a concentration of about between 10 μM to 1000 μM.
Embodiment 135. The pharmaceutical composition of Embodiments 120, wherein the DOT1L inhibitor is an S-adenosyl methionine (SAM) competitive inhibitor.
Embodiment 136. The pharmaceutical composition of Embodiments 120, wherein the DOT1L inhibitor is selected from the group consisting of EPZ004777, pinometostat and SGC0946.
Embodiment 137. The pharmaceutical composition of Embodiments 136, wherein the DOT1L inhibitor is EPZ004777.
Embodiment 138. The pharmaceutical composition of Embodiments 136, wherein the DOT1L inhibitor is pinometostat.
Embodiment 139. The pharmaceutical composition of Embodiments 136, wherein the DOT1L inhibitor is SGC0946.
Embodiment 140. The pharmaceutical composition of Embodiments 137, wherein the is EPZ004777 is at a concentration of about between 0.5 mM to 45 mM.
Embodiment 141. The pharmaceutical composition of Embodiments 138, wherein the pinometostat is at a concentration of about between 0.1 mM to 10 mM.
Embodiment 142. The pharmaceutical composition of Embodiments 139, wherein the SGC0946 is at a concentration of about between 0.5 mM to 5 mM.
Embodiment 143. The pharmaceutical composition of Embodiments 120, wherein the KDM inhibitor is AS 8351, EPT103182 or TC-E 5002.
Embodiment 144. The pharmaceutical composition of Embodiments 143, wherein the KDM inhibitor is AS 8351.
Embodiment 145. The pharmaceutical composition of Embodiments 143, wherein the KDM inhibitor is TC-E 5002.
Embodiment 146. The pharmaceutical composition of Embodiments 144, wherein the AS 8351 is at a concentration of about between 0.5 mM to 5 mM.
Embodiment 147. The pharmaceutical composition of Embodiments 145, wherein the TC-E 5002 is at a concentration of about between 1 mM to 10 mM.
Embodiment 155. The pharmaceutical composition of any of Embodiments 96-154, wherein the pharmaceutical composition is in a biocompatible matrix.
Embodiment 156. The pharmaceutical composition of Embodiments 155, wherein the biocompatible matrix comprises hyaluronic acid, hyaluronates, lecithin gels, pluronics, poly(ethyleneglycol), poloxamers, chitosans, xyloglucans, collagens, fibrins, polyesters, poly(lactides), poly(glycolide), poly(lactic-co-glycolic acid (PLGA), sucrose acetate isobutyrate, glycerol monooleate, poly anhydrides, poly caprolactone sucrose, glycerol monooleate, silk materials, or a combination thereof.
Embodiment 157. The pharmaceutical composition of any of Embodiments 96-156, wherein the pharmaceutical composition is formulated for administration as defined in any of Embodiments 73-95.
Embodiment 158. The pharmaceutical composition any of Embodiments 96-157, for use in treating or preventing an inner ear hearing or balance disorder.
Embodiment 159. The pharmaceutical composition for use according to Embodiments 158, wherein the inner ear hearing or balance disorder is sensorineural hearing loss.
Embodiment 160. Use of the pharmaceutical composition of any of Embodiments 96-159 in the manufacture of a medicament for the treatment or prevention of an inner ear hearing or balance disorder.
Embodiment 161. Use of the pharmaceutical composition according to Embodiments 160, wherein the inner ear hearing or balance disorder is sensorineural hearing loss.
Embodiment 162. A lysine specific demethylase 1 (LSD1) inhibitor for use in treating or preventing an inner ear hearing or balance disorder in a subject, wherein the subject has been, or will be, administered a Wnt agonist.
Embodiment 163. A Wnt agonist for use in treating or preventing an inner ear hearing or balance disorder in a subject, wherein the subject has been, or will be, administered a lysine specific demethylase 1 (LSD1) inhibitor.
Embodiment 164. An epigenetic agent for use in treating or preventing an inner ear hearing or balance disorder in a subject, wherein the subject has been, or will be, administered a lysine specific demethylase 1 (LSD1) inhibitor and a Wnt agonist.
Embodiment 165. The LSD1 inhibitor, Wnt agonist or epigenetic agent for use according to any of Embodiments 160-164, wherein the inner ear hearing or balance disorder is sensorineural hearing loss.
Embodiment 166. The LSD1 inhibitor, Wnt agonist or epigenetic agent for use according to any of Embodiments 160-165, wherein the treatment is as defined in any of Embodiments 9-95.
Embodiment 167. A container comprising a lysine specific demethylase 1 (LSD1) inhibitor and instructions, where those instructions describe the LSD1 inhibitor's use for treating or preventing an inner ear hearing or balance disorder in a subject, wherein the instructions require that the subject has been, or will be, administered a Wnt agonist.
Embodiment 168. A container comprising a Wnt agonist and instructions, where those instructions describe the Wnt agonist's use in treating or preventing an inner ear hearing or balance disorder in a subject, wherein the instructions require that the subject has been, or will be, administered a lysine specific demethylase 1 (LSD1) inhibitor.
Embodiment 169. A container comprising an epigenetic agent and instructions, where those instructions describe the epigenetic agent's use in treating or preventing an inner ear hearing or balance disorder in a subject, wherein the instructions require that the subject has been, or will be, administered a lysine specific demethylase 1 (LSD1) inhibitor and a Wnt agonist.
Embodiment 170. The container according to any of Embodiments 167-169, wherein the inner ear hearing or balance disorder is sensorineural hearing loss.
Embodiment 171. The container according to any of Embodiments 167-169, wherein the treatment is as defined in any of Embodiments 9-95.
Embodiment 172. The method of Embodiments 42, wherein the EZH2 inhibitor is CPI-1169.
Embodiment 173. The method of Embodiments 172, wherein the CPI-169 is at a concentration of about between 1 μM to 10 μM.
Embodiment 174. The pharmaceutical composition of Embodiments 124, wherein the EZH2 inhibitor is CPI-169.
Embodiment 175. The pharmaceutical composition of Embodiments 174, wherein the CPI-169 is at a concentration of about between 1 mM to 10 mM.
Embodiment 176. The method of Embodiments 61, wherein the KDM inhibitor is EPT103182.
Embodiment 177. The method of Embodiments 176, wherein the EPT103182 is at a concentration of about between 1 nM to 100 nM
Embodiment 178. The pharmaceutical composition of Embodiments 143, wherein the KDM inhibitor is EPT103182.
Embodiment 179. The pharmaceutical composition of Embodiments 178, wherein the EPT103182 is at a concentration of about between 1 μM to 100 μM.

### EZH2 Inhibitor+Wnt Agonist Embodiments

Embodiment 1. A method for increasing proliferation of a cochlear supporting cell or a vestibular supporting cell, comprising contacting the supporting cell with:
   a) a first epigenetic agent that is an enhancer of zeste homolog 2 (EZH2) inhibitor; and
   b) a Wnt agonist;
   wherein steps (a) and (b) can occur in any order or simultaneously, thereby increasing cochlear supporting cell or vestibular supporting cell proliferation compared to a vehicle control.
Embodiment 2. A method for producing an expanded population of cochlear or vestibular cells, comprising contacting a population of cochlear supporting cells or vestibular supporting cells with:
   a) a first epigenetic agent that is an enhancer of zeste homolog 2 (EZH2) inhibitor and;
   b) a Wnt agonist
   wherein steps (a) and (b) can occur in any order or simultaneously, thereby producing an expanded population of cochlear or vestibular cells compared to a vehicle control.
Embodiment 3. The method of Embodiment 1 or Embodiment 2, wherein the cochlear supporting cell(s) or vestibular supporting cell(s) express(es) leucine-rich repeat-containing G-protein coupled receptor 5 (Lgr5).
Embodiment 4. The method of any preceding Embodiment, wherein the cochlear supporting cell(s) or vestibular supporting cell(s) are/is a mature cell(s).
Embodiment 5. The method of any of Embodiments 2-4, wherein the expanded population of cochlear or vestibular cells expresses leucine-rich repeat-containing G-protein coupled receptor 5 (Lgr5).
Embodiment 6. The method of any preceding Embodiment, wherein the cochlear supporting cell(s) or vestibular supporting cell(s) are/is a cochlear supporting cell(s).
Embodiment 7. The method of Embodiment 6, wherein the expanded population of cochlear or vestibular cells are cochlear cells.
Embodiment 8. The method of any preceding Embodiment, wherein the EZH2 inhibitor in combination with the Wnt agonist increases the Lgr5 Activity of the expanded population of cochlear or vestibular cells by a factor of at least 10, 20, 30, 40, 50, 75, 100 or 200% compared to a Wnt agonist alone or a Wnt agonist in combination with valproic acid, wherein the Lgr5 Activity is measured in a Stem Cell Proliferation Assay
Embodiment 9. A method of treating a subject who has, or is at risk of, developing an inner ear hearing or balance disorder, comprising administering to the subject:
   a) a first epigenetic agent that is an enhancer of zeste homolog 2 (EZH2) inhibitor; and
   b) a Wnt agonist
   wherein step (a) and (b) can occur in any order or simultaneously.
Embodiment 10. The method of Embodiment 9, wherein the subject has an inner ear hearing or balance disorder.
Embodiment 11. The method of Embodiment 9 or 10, wherein the disorder is an inner ear hearing disorder.
Embodiment 12. The method of Embodiment 9 or 10, wherein the disorder is a balance disorder
Embodiment 13. The method of any of Embodiments 9-12, wherein the inner ear hearing or balance disorder is sensorineural hearing loss.
Embodiment 14. The method of any of Embodiments 9-13, wherein the treatment results in improved auditory function when assessed by behavioural audiometry or auditory brainstem response (ABR) testing.
Embodiment 15. The method of any preceding Embodiment, wherein the EZH2 inhibitor is an enzymatic inhibitor.
Embodiment 16. The method of any preceding Embodiment, wherein the EZH2 inhibitor is selected from the group consisting of: CPI-1205, CPI-169, E11, PF-06821497, tazemetostat, CPI-360, EPZ011989, UNC 2399, PF 06726304, and valemetostat.
Embodiment 17. The method of any preceding Embodiment, wherein the EZH2 inhibitor is CPI-1205.
Embodiment 18. The method of any preceding Embodiment, wherein the EZH2 inhibitor is CPI-169.
Embodiment 19. The method of any preceding Embodiment, wherein the EZH2 inhibitor is E11.
Embodiment 20. The method of any preceding Embodiment, wherein the EZH2 inhibitor is PF-06821497.
Embodiment 21. The method of any preceding Embodiment, wherein the EZH2 inhibitor is tazemetostat.
Embodiment 22. The method of any preceding Embodiment, wherein the EZH2 inhibitor is valemetostat.
Embodiment 23. The method of Embodiment 17, wherein the CPI-1205 is at a concentration of about between 10 nM to 1000 nM.
Embodiment 24. The method of Embodiment 18, wherein the CPI-169 is at a concentration of about between 1 μM to 10 μM.
Embodiment 25. The method of Embodiment 19, wherein the E11 is at a concentration of about between 1 μM to 10 μM.
Embodiment 26. The method of Embodiment 20, wherein the PF-06821497 is at a concentration of about between 1 nM to 100 nM.
Embodiment 27. The method of Embodiment 21, wherein the tazemetostat is at a concentration of about between 0.1 μM to 1.5 μM.
Embodiment 28. The method of Embodiment 22, wherein the valemetostat is at a concentration of about between 10 nM to 1000 nM.
Embodiment 29. The method of any preceding Embodiment, wherein the Wnt agonist is a GSK3 inhibitor.
Embodiment 30. The method of Embodiment 29, wherein the GSK3 inhibitor is selected from the group consisting of: AZD1080, LY2090314, a substituted 3-Imidazo[1,2-a]pyridin-3-yl-4-(1,2,3,4-tetrahydro-[1,4]diazepino-[6,7,1-hi]indol-7-yl)pyrrole-2,5-dione, GSK3 inhibitor XXII or CHIR99021.
Embodiment 31. The method of Embodiment 30, wherein the GSK3 inhibitor is AZD1080.
Embodiment 32. The method of Embodiment 30, wherein the GSK3 inhibitor is IN2090314.
Embodiment 33. The method of Embodiment 30, wherein the GSK3 inhibitor is a substituted 3-Imidazo[1,2-a]pyridin-3-yl-4-(1,2,3,4-tetrahydro-[1,4]diazepino-[6,7,1-hi]indol-7-yl)pyrrole-2,5-dione.
Embodiment 34. The method of Embodiment 30, wherein the GSK3 inhibitor is GSK3 inhibitor XXII.
Embodiment 35. The method of Embodiment 30, wherein the GSK3 inhibitor is CHIR99021.
Embodiment 36. The method of Embodiment 31, wherein AZD1080 is at a concentration of about between 0.5 μM to 5 μM.
Embodiment 37. The method of Embodiment 32, wherein LY2090314 is at a concentration of about between 4 nM to 40 nM.
Embodiment 38. The method of Embodiment 33, wherein a substituted 3-Imidazo[1,2-a]pyridin-3-yl-4-(1,2,3,4-tetrahydro-[1,4]diazepino-[6,7,1-hi]indol-7-yl)pyrrole-2,5-dione is at a concentration of about between 5 nM to 500 nM.
Embodiment 39. The method of Embodiment 34, wherein GSK3 inhibitor XXII at a concentration of about between 0.1 μM to 1 μM.
Embodiment 40. The method of Embodiment 35, wherein CHIR99021 is at a concentration of about between 1 μM to 10 μM.
Embodiment 41. The method of any preceding Embodiment, further comprising contacting the cochlear or vestibular supporting cell(s) with, or administering to the subject, a second epigenetic agent.
Embodiment 42. The method of Embodiment 41, wherein the second epigenetic agent is an HDAC inhibitor, an LSD1 inhibitor, a DOT1L inhibitor, or a KDM inhibitor.
Embodiment 43. The method of Embodiment 42, wherein the HDAC inhibitor is Valproic Acid (VPA)
Embodiment 44. The method of Embodiment 43, wherein the VPA is at a concentration of about between 100 μM to 4,000 μM.
Embodiment 45. The method of Embodiment 42, wherein the LSD1 inhibitor is irreversible.
Embodiment 46. The method of Embodiment 42, wherein the LSD1 inhibitor is selected from the group consisting of GSK-2879552, GSK-LSD1, Tranylcypromine, Phenelzine sulfate, RN-1, or ORY-1001.
Embodiment 47. The method of Embodiment 42, wherein the LSD1 inhibitor is GSK2879552.
Embodiment 48. The method of Embodiment 42, wherein the LSD1 inhibitor is GSK-LSD1.
Embodiment 49. The method of Embodiment 42, wherein the LSD1 inhibitor is Tranylcypromine.
Embodiment 50. The method of Embodiment 42, wherein the LSD1 inhibitor is Phenelzine sulfate.
Embodiment 51. The method of Embodiment 47, wherein GSK2879552 is at a concentration of about between 4 nM to 30 μM.
Embodiment 52. The method of Embodiment 48, wherein GSK-LSD1 is at a concentration of about between 4 nM to 50 μM.
Embodiment 53. The method of Embodiment 49, wherein Tranylcypromine is at a concentration of about between 0.1 μM to 20 μM.
Embodiment 54. The method of Embodiment 50, wherein Phenelzine sulfate at a concentration of about between 0.1 μM to 10 μM.
Embodiment 55. The method of Embodiment 42, wherein the DOT1L inhibitor is an S-adenosyl methionine (SAM) competitive inhibitor.
Embodiment 56. The method of Embodiment 42, wherein the DOT1L inhibitor is selected from the group consisting of EPZ004777, pinometostat and SGC0946.
Embodiment 57. The method of Embodiment 56, wherein the DOT1L inhibitor is EPZ004777.
Embodiment 58. The method of Embodiment 56, wherein the DOT1L inhibitor is pinometostat.
Embodiment 59. The method of Embodiment 56, wherein the DOT1L inhibitor is SGC0946.
Embodiment 60. The method of Embodiment 57, wherein the EPZ004777 is at a concentration of about between 0.5 μM to 45 μM.
Embodiment 61. The method of Embodiment 58, wherein the pinometostat is at a concentration of about between 0.1 μM to 10 μM.
Embodiment 62. The method of Embodiment 59, wherein the SGC0946 is at a concentration of about between 0.5 μM to 5 μM.
Embodiment 63. The method of Embodiment 42, wherein the KDM inhibitor is AS 8351 or TC-E 5002.
Embodiment 64. The method of Embodiment 42, wherein the KDM inhibitor is AS 8351.
Embodiment 65. The method of Embodiment 42, wherein the KDM inhibitor is TC-E 5002.
Embodiment 66. The method of Embodiment 64, wherein the AS 8351 is at a concentration of about between 0.5 μM to 5 μM.
Embodiment 67. The method of Embodiment 65, wherein the TC-E 5002 is at a concentration of about between 0.1 μM to 10 μM.
Embodiment 75. The method of any preceding Embodiment, wherein the EZH2 inhibitor is administered locally and/or systemically.
Embodiment 76. The method of any preceding Embodiment, wherein the EZH2 inhibitor is administered locally.
Embodiment 77. The method of any preceding Embodiment, wherein the EZH2 inhibitor is administered systemically.
Embodiment 78. The method of any preceding Embodiment, wherein the EZH2 inhibitor is administered locally and systemically.
Embodiment 79. The method of any preceding Embodiment, wherein the Wnt agonist is administered locally and/or systemically.
Embodiment 80. The method of any preceding Embodiment, wherein the Wnt agonist is administered locally.
Embodiment 81. The method of any preceding Embodiment, wherein the Wnt agonist is administered systemically.
Embodiment 82. The method of any preceding Embodiment, wherein the Wnt agonist administered locally and systemically.
Embodiment 83 The method of any preceding Embodiment, wherein the second epigenetic agent is administered locally and/or systemically.
Embodiment 84. The method of any preceding Embodiment, wherein the second epigenetic agent is administered locally.
Embodiment 85. The method of any preceding Embodiment, wherein the second epigenetic agent is administered systemically.
Embodiment 86. The method of any preceding Embodiment, wherein the second epigenetic agent is administered locally and systemically.
Embodiment 87. The method of any of Embodiments 75-86, wherein the local administration is to the tympanic membrane, the middle ear or the inner ear.
Embodiment 88. The method of Embodiment 87, wherein the local administration is to the middle ear
Embodiment 89. The method of any of Embodiments 75-86, wherein the systemic administration is oral or parenteral.
Embodiment 90. The method of Embodiment 89, wherein the systemic administration is oral.
Embodiment 91. The method of any of Embodiments 75-90, wherein the EZH2 inhibitor is CPI-1205 and is administered locally at a dose of 1 μM.
Embodiment 92. The method of any of Embodiments 75-90, wherein the EZH2 inhibitor is CPI-169 and is administered locally at a dose of 1 μM.
Embodiment 93. The method of any of Embodiments 75-90, wherein the EZH2 inhibitor is E11 and is administered locally at a dose of 5 μM.
Embodiment 94. The method of any of Embodiments 75-90, wherein the EZH2 inhibitor is tazemetostat and is administered locally at a dose of 0.3 μM.
Embodiment 95. The method of any of Embodiments 75-90, wherein the EZH2 inhibitor is CPI-1205 and is administered systemically at a unit dose of 800 mg.
Embodiment 96. The method of any of Embodiments 75-90, wherein the EZH2 inhibitor is CPI-169 and is administered systemically at a unit dose of 100 mg.
Embodiment 97. The method of any of Embodiments 75-90, wherein the EZH2 inhibitor is E11 and is administered systemically at a unit dose of 100 mg.
Embodiment 98. The method of any of Embodiments 75-90, wherein the EZH2 inhibitor is tazemetostat and is administered systemically at a unit dose of 100 mg.
Embodiment 99. The method of any of Embodiments 75-98, wherein the Wilt agonist is CHIR99021 and is administered locally at a dose of 4 μM.
Embodiment 100. The method of any of Embodiments 75-99, wherein the second epigenetic agent is valproic acid (VPA) and is administered locally at a dose of 1 mM.
Embodiment 101. The method of any of Embodiments 75-99, wherein the second epigenetic agent is valproic acid (VPA) and is administered systemically at a unit dose of 500 mg.
Embodiment 102. A pharmaceutical composition comprising a first epigenetic agent that is an EZH2 inhibitor, a Wnt agonist and a pharmaceutically acceptable carrier.
Embodiment 103. The pharmaceutical composition of Embodiment 102, wherein the EZH2 inhibitor is an enzymatic inhibitor.
Embodiment 104. The pharmaceutical composition of Embodiment 102, wherein the EZH2 inhibitor is selected from the group consisting of: CPI-1205, CPI-169, E11, PF-06821497, tazemetostat, CPI-360, EPZ011989, UNC 2399, PF 06726304, and valemetostat.
Embodiment 105. The pharmaceutical composition of Embodiment 104, wherein the EZH2 inhibitor is CPI-1205.
Embodiment 106. The pharmaceutical composition of Embodiment 104, wherein the EZH2 inhibitor is CPI-169.
Embodiment 107. The pharmaceutical composition of Embodiment 104, wherein the EZH2 inhibitor is E11.
Embodiment 108. The pharmaceutical composition of Embodiment 104, wherein the EZH2 inhibitor is PF-06821497.
Embodiment 109. The pharmaceutical composition of Embodiment 104, wherein the EZH2 inhibitor is tazemetostat.
Embodiment 110. The pharmaceutical composition of Embodiment 104, wherein the EZH2 inhibitor is valemetostat.
Embodiment 111. The pharmaceutical composition of Embodiment 105, wherein the CPI-1205 is at a concentration of about between 10 μM to 1000 μM.
Embodiment 112. The pharmaceutical composition of Embodiment 106, wherein the CPI-169 is at a concentration of about between 1 mM to 10 mM.
Embodiment 113. The pharmaceutical composition of Embodiment 107, wherein the E11 is at a concentration of about between 1 mM to 10 mM.
Embodiment 114. The pharmaceutical composition of Embodiment 108, wherein the PF-06821497 is at a concentration of about between 1 μM to 100 μM.
Embodiment 115. The pharmaceutical composition of Embodiment 109, wherein the tazemetostat is at a concentration of about between 0.1 mM to 10 mM.
Embodiment 116. The pharmaceutical composition of Embodiment 110, wherein the valemetostat is at a concentration of about between 10 μM to 1000 μM.
Embodiment 117. The pharmaceutical composition of any of Embodiments 102-116, wherein the Wnt agonist is a GSK3 inhibitor.
Embodiment 118. The pharmaceutical composition of Embodiment 117, wherein the GSK3 inhibitor is selected from the group consisting of: AZD1080, LY2090314, a substituted 3-Imidazo[1,2-a]pyridin-3-yl-4-(1,2,3,4-tetrahydro-[1,4]diazepino-[6,7,1-hi]indol-7-yl)pyrrole-2,5-dione, GSK3 inhibitor XXII or CHIR99021.
Embodiment 119. The pharmaceutical composition of Embodiment 118, wherein the GSK3 inhibitor is AZD1080.
Embodiment 120. The pharmaceutical composition of Embodiment 118, wherein the GSK3 inhibitor is LY2090314.
Embodiment 121. The pharmaceutical composition of Embodiment 118, wherein the GSK3 inhibitor is a substituted 3-Imidazo[1,2-a]pyridin-3-yl-4-(1,2,3,4-tetrahydro-[1,4]diazepino-[6,7,1-hi]indol-7-yl)pyrrole-2,5-dione.
Embodiment 122. The method of Embodiment 118, wherein wherein the GSK3 inhibitor is GSK3 inhibitor XXII.
Embodiment 123. The pharmaceutical composition of Embodiment 118, wherein the GSK3 inhibitor is CHIR99021.
Embodiment 124. The pharmaceutical composition of Embodiment 119, wherein AZD1080 is at a concentration of about between 0.5 mM to 5 mM.
Embodiment 125. The pharmaceutical composition of Embodiment 120, wherein LY2090314 is at a concentration of about between 4 μM to 40 μM.
Embodiment 126. The pharmaceutical composition of Embodiment 121, wherein a substituted 3-Imidazo[1,2-a]pyridin-3-yl-4-(1,2,3,4-tetrahydro-[1,4]diazepino-[6,7,1-hi]indol-7-yl)pyrrole-2,5-dione is at a concentration of about between 5 μM to 500 μM.
Embodiment 127. The pharmaceutical composition of Embodiment 122, wherein GSK3 inhibitor XXII at a concentration of about between 0.1 mM to 1 mM.
Embodiment 128. The pharmaceutical composition of Embodiment 123, wherein CHIR99021 is at a concentration of about between 1 mM to 10 mM.
Embodiment 129. The pharmaceutical composition of any of Embodiments 102-128 further comprising a second epigenetic agent.
Embodiment 130. The pharmaceutical composition of Embodiment 129, wherein the second epigenetic agent is an HDAC inhibitor, an LSD1 inhibitor, a DOT1L inhibitor, or a KDM inhibitor.
Embodiment 131. The pharmaceutical composition of Embodiment 130, wherein the HDAC inhibitor is Valproic Acid (VPA).
Embodiment 132. The pharmaceutical composition of Embodiment 131, wherein the VPA is at a concentration of about between 100 mM to 4,000 mM.
Embodiment 133. The pharmaceutical composition of Embodiment 130, wherein the LSD1 inhibitor is irreversible.
Embodiment 134. The pharmaceutical composition Embodiment 130, wherein the LSD1 inhibitor is selected from the group consisting of GSK-2879552, GSK-LSD1, RN-1, ORY-1001, Tranylcypromine and Phenelzine sulfate.
Embodiment 135. The pharmaceutical composition Embodiment 134, wherein the LSD1 inhibitor is GSK2879552.
Embodiment 136. The pharmaceutical composition Embodiment 134, wherein the LSD1 inhibitor is GSK-LSD1.
Embodiment 137. The pharmaceutical composition Embodiment 134, wherein the LSD1 inhibitor is Tranylcypromine.
Embodiment 138. The pharmaceutical composition Embodiment 134, wherein the LSD1 inhibitor is Phenelzine sulfate.
Embodiment 139. The pharmaceutical composition Embodiment 135, wherein GSK2879552 is at a concentration of about between 4 μM to 30 mM.
Embodiment 140. The pharmaceutical composition Embodiment 136, wherein GSK-LSD1 is at a concentration of about between 4 μM to 50 mM.
Embodiment 141. The pharmaceutical composition Embodiment 137, wherein Tranylcypromine is at a concentration of about between 0.1 mM to 20 mM.
Embodiment 142. The pharmaceutical composition Embodiment 138, wherein Phenelzine sulfate is at a concentration of about between 0.1 mM to 10 mM.
Embodiment 143. The pharmaceutical composition of Embodiment 130, wherein the DOT1L inhibitor is an S-adenosyl methionine (SAM) competitive inhibitor.
Embodiment 144. The pharmaceutical composition of Embodiment 130, wherein the DOT1L inhibitor is selected from the group consisting of EPZ004777, pinometostat and SGC0946.
Embodiment 145. The pharmaceutical composition of Embodiment 144, wherein the DOT1L inhibitor is EPZ004777.
Embodiment 146. The pharmaceutical composition of Embodiment 144, wherein the DOT1L inhibitor is pinometostat.
Embodiment 147. The pharmaceutical composition of Embodiment 144, wherein the DOT1L inhibitor is SGC0946.
Embodiment 148. The pharmaceutical composition of Embodiment 145, wherein the is EPZ004777 is at a concentration of about between 0.5 mM to 45 mM.
Embodiment 149. The pharmaceutical composition of Embodiment 146, wherein the pinometostat is at a concentration of about between 0.1 mM to 10 mM.
Embodiment 150. The pharmaceutical composition of Embodiment 147, wherein the SGC0946 is at a concentration of about between 0.5 mM to 5 mM.
Embodiment 151. The pharmaceutical composition of Embodiment 130, wherein the KDM inhibitor is AS 8351 or TC-E 5002.
Embodiment 152. The pharmaceutical composition of Embodiment 151, wherein the KDM inhibitor is AS 8351.
Embodiment 153. The pharmaceutical composition of Embodiment 151, wherein the KDM inhibitor is TC-E 5002.
Embodiment 154. The pharmaceutical composition of Embodiment 152, wherein the AS 8351 is at a concentration of about between 0.5 mM to 5 mM.
Embodiment 155. The pharmaceutical composition of Embodiment 153, wherein the TC-E 5002 is at a concentration of about between 1 mM to 10 mM.
Embodiment 163. The pharmaceutical composition of any of Embodiments 102-162, wherein the pharmaceutical composition is in a biocompatible matrix.
Embodiment 164. The pharmaceutical composition of Embodiment 163, wherein the biocompatible matrix comprises hyaluronic acid, hyaluronates, lecithin gels, pluronics, poly(ethyleneglycol), poloxamers, chitosans, xyloglucans, collagens, fibrins, polyesters, poly(lactides), poly(glycolide), poly(lactic-co-glycolic acid (PLGA), sucrose acetate isobutyrate, glycerol monooleate, poly anhydrides, poly caprolactone sucrose, glycerol monooleate, silk materials, or a combination thereof.
Embodiment 165. The pharmaceutical composition of any of Embodiments 102-164, wherein the pharmaceutical composition is formulated for administration as defined in any of Embodiments 75-101.
Embodiment 166. The pharmaceutical composition any of Embodiments 102-165, for use in treating or preventing an inner ear hearing or balance disorder.
Embodiment 167. The pharmaceutical composition for use according to Embodiment 166, wherein the inner ear hearing or balance disorder is sensorineural hearing loss.
Embodiment 168. Use of the pharmaceutical composition of any of Embodiments 102-167 in the manufacture of a medicament for the treatment or prevention of an inner ear hearing or balance disorder.
Embodiment 169. Use of the pharmaceutical composition according to Embodiment 168, wherein the inner ear hearing or balance disorder is sensorineural hearing loss.
Embodiment 170. An enhancer of zeste homolog 2 (EZH2) inhibitor for use in treating or preventing an inner ear hearing or balance disorder in a subject, wherein the subject has been, or will be, administered a Wnt agonist.
Embodiment 171. A Wnt agonist for use in treating or preventing an inner ear hearing or balance disorder in a subject, wherein the subject has been, or will be, administered an enhancer of zeste homolog 2 (EZH2) inhibitor.
Embodiment 172. An epigenetic agent for use in treating or preventing an inner ear hearing or balance disorder in a subject, wherein the subject has been, or will be, administered an enhancer of zeste homolog 2 (EZH2) inhibitor and a Wnt agonist.
Embodiment 173. The EZH2 inhibitor, Wnt agonist or epigenetic agent for use according to any of Embodiments 170-172, wherein the inner ear hearing or balance disorder is sensorineural hearing loss.
Embodiment 174. The EZH2 inhibitor, Wnt agonist or epigenetic agent for use according to any of Embodiments 170-173, wherein the treatment is as defined in any of Embodiments 9-101.
Embodiment 175. A container comprising an enhancer of zeste homolog 2 (EZH2) inhibitor and instructions, where those instructions describe the EZH2 inhibitor's use for treating or preventing an inner ear hearing or balance disorder in a subject, wherein the instructions require that the subject has been, or will be, administered a Wnt agonist.
Embodiment 176. A container comprising a Wnt agonist and instructions, where those instructions describe the Wnt agonist's use in treating or preventing an inner ear hearing or balance disorder in a subject, wherein the instructions require that the subject has been, or will be, administered an enhancer of zeste homolog 2 (EZH2) inhibitor.
Embodiment 177. A container comprising an epigenetic agent and instructions, where those instructions describe the epigenetic agent's use in treating or preventing an inner ear hearing or balance disorder in a subject, wherein the instructions require that the subject has been, or will be, administered an enhancer of zeste homolog 2 (EZH2) inhibitor and a Wnt agonist.
Embodiment 178. The container according to any of Embodiments 175-177, wherein the inner ear hearing or balance disorder is sensorineural hearing loss.
Embodiment 179. The container according to any of Embodiments 175-177, wherein the treatment is as defined in any of Embodiments 9-101.

### DOT1L Inhibtor+Wnt Agonist Embodiments

Embodiment 1. A method for increasing proliferation of a cochlear supporting cell or a vestibular supporting cell, comprising contacting the supporting cell with:
   a) a first epigenetic agent that is a disruptor of telomeric silencing 1-like (DOT1L) inhibitor; and
   b) a Wnt agonist;
   wherein steps (a) and (b) can occur in any order or simultaneously, thereby increasing cochlear supporting cell or vestibular supporting cell proliferation compared to a vehicle control.
Embodiment 2. A method for producing an expanded population of cochlear or vestibular cells, comprising contacting a population of cochlear supporting cells or vestibular supporting cells with:
   a) a first epigenetic agent that is a disruptor of telomeric silencing 1-like (DOT1L) inhibitor and;
   b) a Wnt agonist
   wherein steps (a) and (b) can occur in any order or simultaneously, thereby producing an expanded population of cochlear or vestibular cells compared to a vehicle control.
Embodiment 3. The method of Embodiment 1 or Embodiment 2, wherein the cochlear supporting cell(s) or vestibular supporting cell(s) express(es) leucine-rich repeat-containing G-protein coupled receptor 5 (Lgr5).
Embodiment 4. The method of any preceding Embodiment, wherein the cochlear supporting cell(s) or vestibular supporting cell(s) are/is a mature cell(s).
Embodiment 5. The method of any of Embodiments 2-4, wherein the expanded population of cochlear or vestibular cells expresses leucine-rich repeat-containing G-protein coupled receptor 5 (Lgr5).
Embodiment 6. The method of any preceding Embodiment, wherein the cochlear supporting cell(s) or vestibular supporting cell(s) are/is a cochlear supporting cell(s).
Embodiment 7. The method of Embodiment 6, wherein the expanded population of cochlear or vestibular cells are cochlear cells.
Embodiment 8. The method of any preceding Embodiment, wherein the DOT1L inhibitor in combination with the Wnt agonist increases the Lgr5 Activity of the expanded population of cochlear or vestibular cells by a factor of at leak 10, 20, 30, 40, 50, 75, 100 or 200% compared to a Wnt agonist alone or a Wnt agonist in combination with valproic acid, wherein the Lgr5 Activity is measured in a Stem Cell Proliferation Assay.
Embodiment 9. A method of treating a subject who has, or is at risk of, developing an inner ear hearing or balance disorder, comprising administering to the subject:
   a) a first epigenetic agent that is a disruptor of telomeric silencing 1-like (DOT1L) inhibitor; and
   b) a Wnt agonist
   wherein step (a) and (b) can occur in any order or simultaneously.
Embodiment 10. The method of Embodiment 9, wherein the subject has an inner ear hearing or balance disorder.
Embodiment 11. The method of Embodiment 9 or 10, wherein the disorder is an inner ear hearing disorder.
Embodiment 12. The method of Embodiment 9 or 10, wherein the disorder is a balance disorder.
Embodiment 13. The method of any of Embodiments 9-12, wherein the inner ear hearing or balance disorder is sensorineural hearing loss.
Embodiment 14. The method of any of Embodiments 9-13, wherein the treatment results in improved auditory function at one or more frequencies when assessed by behavioural audiometry or auditory brainstem response (ABR) testing.
Embodiment 15. The method of any preceding Embodiment, wherein the DOT1L inhibitor is an S-adenosyl methionine (SAM) competitive inhibitor.
Embodiment 16. The method of any preceding Embodiment, wherein the DOT1L inhibitor is selected from the group consisting of EPZ004777, pinometostat, and SGC0946.
Embodiment 17. The method of Embodiment 16, wherein the DOT1L inhibitor is EPZ004777.
Embodiment 18. The method of Embodiment 16, wherein the DOT1L inhibitor is pinometostat
Embodiment 19. The method of Embodiment 16, wherein the DOT1L inhibitor is SGC0946.
Embodiment 20. The method of Embodiment 17, wherein the EPZ004777 is at a concentration of about between 0.1 μM to 45 μM.
Embodiment 21. The method of Embodiment 18, wherein the pinometostat is at a concentration of about between 0.1 μM to 10 μM.
Embodiment 22. The method of Embodiment 19, wherein the SGC0946 is at a concentration of about between 0.6 μM to 5 μM.
Embodiment 23. The method of any preceding Embodiment, wherein the Wnt agonist is a GSK3 inhibitor.
Embodiment 24. The method of Embodiment 23, wherein the GSK3 inhibitor is selected from the group consisting of: AZD1080, LY2090314, a substituted 3-Imidazo[1,2-a]pyridin-3-yl-4-(1,2,3,4-tetrahydro-[1,4]diazepino-[6,7,1-hi]indol-7-yl)pyrrole-2,5-dione, GSK3 inhibitor XXII or CHIR99021.
Embodiment 25. The method of Embodiment 24, wherein the GSK3 inhibitor is AZD1080.
Embodiment 26. The method of Embodiment 24, wherein the GSK3 inhibitor is LY2090314.
Embodiment 27. The method of Embodiment 24, wherein the GSK3 inhibitor is a substituted 3-Imidazo[1,2-a]pyridin-3-yl-4-(1,2,3,4-tetrahydro-[1,4]diazepino-[6,7,1-hi]indol-7-yl)pyrrole-2,5-dione.
Embodiment 28. The method of Embodiment 24, wherein the GSK3 inhibitor is GSK3 inhibitor XXII.
Embodiment 29. The method of Embodiment 24, wherein the GSK3 inhibitor is CHIR99021.
Embodiment 30. The method of Embodiment 25, wherein AZD1080 is at a concentration of about between 0.5 μM to 5 μM.
Embodiment 31. The method of Embodiment 26, wherein LY2090314 is at a concentration of about between 4 nM to 40 nM.
Embodiment 32. The method of Embodiment 27, wherein a substituted 3-Imidazo[1,2-a]pyridin-3-yl-4-(1,2,3,4-tetrahydro-[1,4]diazepino-[6,7,1-hi]indol-7-yl)pyrrole-2,5-dione is at a concentration of about between 5 nM to 500 nM.
Embodiment 33. The method of Embodiment 28, wherein GSK3 inhibitor XXII at a concentration of about between 0.1 μM to 1 μM.
Embodiment 34. The method of Embodiment 29, wherein CHIR99021 is at a concentration of about between 1 μM to 10 μM.
Embodiment 35. The method of any preceding Embodiment, further comprising contacting the cochlear or vestibular supporting cell(s) with, or administering to the subject, a second epigenetic agent.
Embodiment 36. The method of Embodiment 35, wherein the second epigenetic agent is an HDAC inhibitor, an EZH2 inhibitor, a LSD1 inhibitor, or a KDM inhibitor.
Embodiment 37. The method of Embodiment 36, wherein the HDAC inhibitor is Valproic Acid (VPA)
Embodiment 38. The method of Embodiment 37, wherein the VPA is at a concentration of about between 100 μM to 4,000 μM.
Embodiment 39. The method of Embodiment 36, wherein the EZH2 inhibitor an enzymatic inhibitor.
Embodiment 40. The method of Embodiment 36, wherein the EZH2 inhibitor is selected from the group consisting of: CPI-1205, E11, PF-06821497, tazemetostat, valemetostat CPI-169, CPI-360, EPZ011989, UNC 2399, and PF 06726304.
Embodiment 41. The method of Embodiment 40, wherein the EZH2 inhibitor is CPI-1205.
Embodiment 42. The method of Embodiment 40, wherein the EZH2 inhibitor is E11.
Embodiment 43. The method of Embodiment 40, wherein the EZH2 inhibitor is PF-06821497.
Embodiment 44. The method of Embodiment 40, wherein the EZH2 inhibitor is tazemetostat.
Embodiment 45. The method of Embodiment 40, wherein the EZH2 inhibitor is valemetostat.
Embodiment 46. The method of Embodiment 40, wherein the EZH2 inhibitor is CPI-169.
Embodiment 47. The method of Embodiment 41, wherein the CPI-1205 is at a concentration of about between 10 nM to 1000 nM.
Embodiment 48. The method of Embodiment 42, wherein the E11 is at a concentration of about between 10 μM to 100 μM.
Embodiment 49. The method of Embodiment 43, wherein the PF-06821497 is at a concentration of about between 1 nM to 100 nM.
Embodiment 50. The method of Embodiment 44, wherein the tazemetostat is at a concentration of about between 0.1 μM to 1.5 μM.
Embodiment 51. The method of Embodiment 45, wherein the valemetostat is at a concentration of about between 10 μM to 1000 μM.
Embodiment 52. The method of Embodiment 46, wherein the CPI-169 is at a concentration of about between 1 μM to 10 μM.
Embodiment 53. The method of any preceding Embodiment, wherein the LSD1 inhibitor is irreversible.
Embodiment 54. The method of Embodiment 36, wherein the LSD1 inhibitor is selected from the group consisting of GSK-2879552, GSK-LSD1, Tranylcypromine, Phenelzine sulfate, RN-1, and ORY-1001.
Embodiment 55. The method of Embodiment 54, wherein the LSD1 inhibitor is GSK2879552.
Embodiment 56. The method of Embodiment 54, wherein the LSD1 inhibitor is GSK-LSD1.
Embodiment 57. The method of Embodiment 54, wherein the LSD1 inhibitor is Tranylcypromine.
Embodiment 58. The method of Embodiment 54, wherein the LSD1 inhibitor is Phenelzine sulfate.
Embodiment 59. The method of Embodiment 55, wherein GSK2879552 is at a concentration of about between 4 nM to 30 μM.
Embodiment 60. The method of Embodiment 56, wherein GSK-LSD1 is at a concentration of about between 4 nM to 50 μM.
Embodiment 61. The method of Embodiment 57, wherein Tranylcypromine is at a concentration of about between 0.1 μM to 20 μM.
Embodiment 62. The method of Embodiment 58, wherein Phenelzine sulfate at a concentration of about between 0.1 μM to 10 μM.
Embodiment 63. The method of Embodiment 36, wherein the KDM inhibitor is AS 8351, TC-E 5002, or EPT-103182.
Embodiment 64. The method of Embodiment 63, wherein the KDM inhibitor is AS 8351.
Embodiment 65. The method of Embodiment 63, wherein the KDM inhibitor is TC-E 5002.
Embodiment 66. The method of Embodiment 63, wherein the KDM inhibitor is EPT-103182.
Embodiment 67. The method of Embodiment 64, wherein the AS 8351 is at a concentration of about between 0.5 μM to 5 μM.
Embodiment 68. The method of Embodiment 65, wherein the TC-E 5002 is at a concentration of about between 0.1 μM to 10 μM.
Embodiment 69. The method of Embodiment 66, wherein the EPT-103182 is at a concentration of about between 1 nM to 100 nM.
Embodiment 77. The method of any preceding Embodiment, wherein the DOT1L inhibitor is administered locally and/or systemically.
Embodiment 78. The method of any preceding Embodiment, wherein the DOT1L inhibitor is administered locally.
Embodiment 79. The method of any preceding Embodiment, wherein the DOT1L inhibitor is administered systemically.
Embodiment 80. The method of any preceding Embodiment, wherein the DOT1L inhibitor is administered locally and systemically.
Embodiment 81. The method of any preceding Embodiment, wherein the Wnt agonist is administered locally and/or systemically.
Embodiment 82. The method of any preceding Embodiment, wherein the Wnt agonist is administered locally.
Embodiment 83. The method of any preceding Embodiment, wherein the Wnt agonist is administered systemically.
Embodiment 84. The method of any preceding Embodiment, wherein the Wnt agonist is administered locally and systemically.
Embodiment 85. The method of any preceding Embodiment, wherein the second epigenetic agent is administered locally and/or systemically.
Embodiment 86. The method of any preceding Embodiment, wherein the second epigenetic agent is administered locally.
Embodiment 87. The method of any preceding Embodiment, wherein the second epigenetic agent is administered systemically.
Embodiment 88. The method of any preceding Embodiment, wherein the second epigenetic agent is administered locally and systemically.
Embodiment 89. The method of any of Embodiments 77-88, wherein the local administration is to the tympanic membrane, the middle ear or the inner ear.
Embodiment 90. The method of Embodiment 89, wherein the local administration is to the middle ear
Embodiment 91. The method of any of Embodiments 77-88, wherein the systemic administration is oral or parenteral.
Embodiment 92. The method of Embodiment 91, wherein the systemic administration is oral.
Embodiment 93. The method of any of Embodiments 77-92, wherein the DOT1L inhibitor is EPZ004777.
Embodiment 94. The method of any of Embodiments 77-92, wherein the DOT1L inhibitor is pinometostat.
Embodiment 95. The method of any of Embodiments 77-92, wherein the DOT1L inhibitor is SGC0946.
Embodiment 96. The method of Embodiment 93, wherein the EPZ004777 is administered systemically at a dose of 50 mg.
Embodiment 97. The method of Embodiment 93, wherein the EPZ004777 is administered locally at a dose of 15 μM.
Embodiment 98. The method of Embodiment 94, wherein the pinometostat is administered systemically at a dose of 60 mg.
Embodiment 99. The method of Embodiment 94, wherein the pinometostat is administered locally at a dose of 10 μM.
Embodiment 100. The method of Embodiment 95, wherein the SGC0946 is administered systemically at a dose of 50 mg.
Embodiment 101. The method of Embodiment 95, wherein the SGC0946 is administered locally at a dose of 1.7 μM.
Embodiment 102. The method of any of Embodiments 77-101, wherein the Wnt agonist is CHIR99021 and is administered locally at a dose of 4 μM.
Embodiment 103. The method of any of Embodiments 77-102, wherein the second epigenetic agent is valproic acid (VPA) and is administered locally at a dose of 1 mM
Embodiment 104. The method of any of Embodiments 77-102, wherein the second epigenetic agent is valproic acid (VPA) and is administered systemically at a unit dose of 500 mg.
Embodiment 105. A pharmaceutical composition comprising a first epigenetic agent that is a DOT1L inhibitor, a Wnt agonist, and a pharmaceutically acceptable carrier.
Embodiment 106. The pharmaceutical composition of Embodiment 105, wherein the DOT1L inhibitor is an S-adenosyl methionine (SAM) competitive inhibitor.
Embodiment 107. The pharmaceutical composition of Embodiment 105, wherein the DOT1L inhibitor is selected from the group consisting of EPZ004777, pinometostat and SGC0946.
Embodiment 108. The pharmaceutical composition of Embodiment 107, wherein the DOT1L inhibitor is EPZ004777.
Embodiment 109. The pharmaceutical composition of Embodiment 107, wherein the DOT1L inhibitor is pinometostat.
Embodiment 110. The pharmaceutical composition of Embodiment 107, wherein the DOT1L inhibitor is SGC0946.
Embodiment 111. The pharmaceutical composition of Embodiment 108, wherein the EPZ004777 is at a concentration of about between 0.5 mM to 45 mM.
Embodiment 112. The pharmaceutical composition of Embodiment 109, wherein the pinometostat is at a concentration of about between 0.1 mM to 10 mM.
Embodiment 113. The pharmaceutical composition of Embodiment 110, wherein the SGC0946 is at a concentration of about between 0.5 mM to 5 mM.
Embodiment 114. The pharmaceutical composition of any one of Embodiments 105-113, wherein the Wnt agonist is a GSK3 inhibitor.
Embodiment 115. The pharmaceutical composition of Embodiment 114, wherein the GSK3 inhibitor is selected from the group consisting of: AZD1080, LY2090314, a substituted 3-Imidazo[1,2-a]pyridin-3-yl-4-(1,2,3,4-tetrahydro-[1,4]diazepino-[6,7,1-hi]indol-7-yl)pyrrole-2,5-dione, GSK3 inhibitor XXII or CHIR99021.
Embodiment 116. The pharmaceutical composition of Embodiment 115, wherein the GSK3 inhibitor is AZD1080.
Embodiment 117. The pharmaceutical composition of Embodiment 115, wherein the GSK3 inhibitor is LY2090314.

Embodiment 118. The pharmaceutical composition of Embodiment 115, wherein the GSK3 inhibitor is a substituted 3-Imidazo[1,2-a]pyridin-3-yl-4-(1,2,3,4-tetrahydro-[1,4]diazepino-[6,7,1-hi]indol-7-yl)pyrrole-2,5-dione.
Embodiment 119. The pharmaceutical composition of Embodiment 115, wherein the GSK3 inhibitor is GSK3 inhibitor XXII.
Embodiment 120. The pharmaceutical composition of Embodiment 115, wherein the GSK3 inhibitor is CHIR99021.
Embodiment 121. The pharmaceutical composition of Embodiment 116, wherein AZD1080 is at a concentration of about between 0.5 mM to 5 mM.
Embodiment 122. The pharmaceutical composition of Embodiment 117, wherein LY2090314 is at a concentration of about between 4 μM to 40 μM.
Embodiment 123. The pharmaceutical composition of Embodiment 118, wherein a substituted 3-Imidazo[1,2-a]pyridin-3-yl-4-(1,2,3,4-tetrahydro-[1,4]diazepino-[6,7,1-hi]indol-7-yl)pyrrole-2,5-dione is at a concentration of about between 5 μM to 500 μM.
Embodiment 124. The pharmaceutical composition of Embodiment 119, wherein GSK3 inhibitor XXII at a concentration of about between 0.1 mM to 1 mM.
Embodiment 125. The pharmaceutical composition of Embodiment 120, wherein CHIR99021 is at a concentration of about between 1 mM to 10 mM.
Embodiment 126. The pharmaceutical composition of any one of Embodiments 105-125 further comprising further comprising a second epigenetic agent.
Embodiment 127. The pharmaceutical composition of Embodiment 126, wherein the second epigenetic agent is an HDAC inhibitor, an EZH2 inhibitor, a LSD1 inhibitor, or a KDM inhibitor.
Embodiment 128. The pharmaceutical composition of Embodiment 127, wherein the HDAC inhibitor is Valproic Acid (VPA)
Embodiment 129. The pharmaceutical composition of Embodiment 128, wherein the VPA is at a concentration of about between 100 mM to 4,000 mM.
Embodiment 130. The pharmaceutical composition of Embodiment 127, wherein the EZH2 inhibitor an enzymatic inhibitor.
Embodiment 131. The pharmaceutical composition of Embodiment 127, wherein the EZH2 inhibitor is selected from the group consisting of: CPI-1205, E11, PF-06821497, tazemetostat, valemetostat, CPI-169 CPI-360, EPZ011989, UNC 2399, and PF 06726304.
Embodiment 132. The pharmaceutical composition of Embodiment 131, wherein the EZH2 inhibitor is CPI-1205.
Embodiment 133. The pharmaceutical composition of Embodiment 131, wherein the EZH2 inhibitor is E11.
Embodiment 134. The pharmaceutical composition of Embodiment 131, wherein the EZH2 inhibitor is PF-06821497.
Embodiment 135. The pharmaceutical composition of Embodiment 131, wherein the EZH2 inhibitor is tazemetostat.
Embodiment 136. The pharmaceutical composition of Embodiment 131, wherein the EZH2 inhibitor is valemetostat.
Embodiment 137. The pharmaceutical composition of Embodiment 131, wherein the EZH2 inhibitor is CPI-169.
Embodiment 138. The pharmaceutical composition of Embodiment 132, wherein the CPI-1205 is at a concentration of about between 10 μM to 1000 μM.
Embodiment 139. The pharmaceutical composition of Embodiment 133, wherein the E11 is at a concentration of about between 1 mM to 100 mM.
Embodiment 140. The pharmaceutical composition of Embodiment 134, wherein the PF-06821497 is at a concentration of about between 1 mM to 100 μM.
Embodiment 141. The pharmaceutical composition of Embodiment 135, wherein the tazemetostat is at a concentration of about between 0.1 mM to 10 mM.
Embodiment 142. The pharmaceutical composition of Embodiment 136, wherein the valemetostat is at a concentration of about between 10 μM to 1000 μM.
Embodiment 143. The pharmaceutical composition of Embodiment 137, wherein the CPI-169 is at a concentration of about between 1 mM to 10 mM.
Embodiment 144. The pharmaceutical composition Embodiment 127, wherein the LSD1 inhibitor is selected from the group consisting of GSK-2879552, GSK-LSD1, Tranylcypromine, Phenelzine sulfate, RN-1, or ORY-1001.

Embodiment 145. The pharmaceutical composition Embodiment 144, wherein the LSD1 inhibitor is GSK2879552.
Embodiment 146. The pharmaceutical composition Embodiment 144, wherein the LSD1 inhibitor is GSK-LSD1.
Embodiment 147. The pharmaceutical composition Embodiment 144, wherein the LSD1 inhibitor is Tranylcypromine.
Embodiment 148. The pharmaceutical composition Embodiment 144, wherein the LSD1 inhibitor is Phenelzine sulfate.
Embodiment 149. The pharmaceutical composition Embodiment 145, wherein GSK2879552 is at a concentration of about between 4 nM to 30 μM.
Embodiment 150. The pharmaceutical composition Embodiment 146, wherein GSK-LSD1 is at a concentration of about between 4 nM to 50 μM.
Embodiment 151. The pharmaceutical composition Embodiment 147, wherein Tranylcypromine is at a concentration of about between 0.1 μM to 20 μM.
Embodiment 152. The pharmaceutical composition Embodiment 148, wherein Phenelzine sulfate is at a concentration of about between 0.1 μM to 10 μM.
Embodiment 153. The pharmaceutical composition of Embodiment 127, wherein the KDM inhibitor is AS 8351, TC-E 5002, or EPT-103182.
Embodiment 154. The pharmaceutical composition of Embodiment 153, wherein the KDM inhibitor is AS 8351.
Embodiment 155. The pharmaceutical composition of Embodiment 153, wherein the KDM inhibitor is TC-E 5002.
Embodiment 156. The pharmaceutical composition of Embodiment 153, wherein the KDM inhibitor is EPT-103182.
Embodiment 157. The pharmaceutical composition of Embodiment 154, wherein the AS 8351 is at a concentration of about between 0.5 μM to 5 μM.
Embodiment 158. The pharmaceutical composition of Embodiment 155, wherein the TC-E 5002 is at a concentration of about between 0.1 μM to 10 μM.
Embodiment 159. The pharmaceutical composition of Embodiment 155, wherein the EPT-103182 is at a concentration of about between 1 nM to 100 nM.
Embodiment 167. The pharmaceutical composition of any of Embodiments 105-166, wherein the pharmaceutical composition is in a biocompatible matrix.
Embodiment 168. The pharmaceutical composition of Embodiment 167, wherein the biocompatible matrix comprises hyaluronic acid, hyaluronates, lecithin gels, pluronics, poly(ethyleneglycol), poloxamers, chitosans, xyloglucans, collagens, fibrins, polyesters, poly(lactides), poly(glycolide), poly(lactic-co-glycolic acid (PLGA), sucrose acetate isobutyrate, glycerol monooleate, poly anhydrides, poly caprolactone sucrose, glycerol monooleate, silk materials, or a combination thereof.
Embodiment 169. The pharmaceutical composition of any of Embodiments 105-168, wherein the pharmaceutical composition is formulated for administration as defined in any of Embodiments 73-94
Embodiment 170. The pharmaceutical composition any of Embodiments 105-169, for use in treating or preventing an inner ear hearing or balance disorder.
Embodiment 171. The pharmaceutical composition for use according to Embodiment 170, wherein the inner ear hearing or balance disorder is sensorineural hearing loss.
Embodiment 172. Use of the pharmaceutical composition of any of Embodiments 105-171 in the manufacture of a medicament for the treatment or prevention of an inner ear hearing or balance disorder.
Embodiment 173. Use of the pharmaceutical composition according to Embodiment 172, wherein the inner ear hearing or balance disorder is sensorineural hearing loss.
Embodiment 174. A disruptor of telomeric silencing 1-like (DOT1L) inhibitor for use in treating or preventing an inner ear hearing or balance disorder in a subject, wherein the subject has been, or will be, administered a Wnt agonist.
Embodiment 175. A Wnt agonist for use in treating or preventing an inner ear hearing or balance disorder in a subject, wherein the subject has been, or will be, administered a disruptor of telomeric silencing 1-like (DOT1L) inhibitor.
Embodiment 176. An epigenetic agent for use in treating or preventing an inner ear hearing or balance disorder in a subject, wherein the subject has been, or will be, administered a disruptor of telomeric silencing 1-like (DOT1L) inhibitor and a Wnt agonist.
Embodiment 177. The DOT1L inhibitor, Wnt agonist or epigenetic agent for use according to any of Embodiments 172-176, wherein the inner ear hearing or balance disorder is sensorineural hearing loss.
Embodiment 178. The DOT1L inhibitor, Wnt agonist or epigenetic agent for use according to any of Embodiments 172-177, wherein the treatment is as defined in any of Embodiments 9-104.
Embodiment 179. A container comprising a disruptor of telomeric silencing 1-like (DOT1L) inhibitor and instructions, where those instructions describe the DOT1L inhibitor's use for treating or preventing an inner ear hearing or balance disorder in a subject, wherein the instructions require that the subject has been, or will be, administered a Wnt agonist.
Embodiment 180. A container comprising a Wnt agonist and instructions, where those instructions describe the Wnt agonist's use in treating or preventing an inner ear hearing or balance disorder in a subject, wherein the instructions require that the subject has been, or will be, administered a disruptor of telomeric silencing 1-like (DOT1L) inhibitor.
Embodiment 181. A container comprising an epigenetic agent and instructions, where those instructions describe the epigenetic agent's use in treating or preventing an inner ear hearing or balance disorder in a subject, wherein the instructions require that the subject has been, or will be, administered a disruptor of telomeric silencing 1-like (DOT1L) inhibitor and a Wnt agonist.
Embodiment 182. The container according to any of Embodiments 179-181, wherein the inner ear hearing or balance disorder is sensorineural hearing loss.
Embodiment 183. The container according to any of Embodiments 179-181, wherein the treatment is as defined in any of Embodiments 9-104.

### KDM Inhibitor+Wnt Agonist Embodiments

Embodiment 1. A method for increasing proliferation of a cochlear supporting cell or a vestibular supporting cell, comprising contacting the supporting cell with:
   a) a first epigenetic agent that is a histone lysine demethylase (KDM) inhibitor; and
   b) a Wnt agonist;
   wherein steps (a) and (b) can occur in any order or simultaneously, thereby increasing cochlear supporting cell or vestibular supporting cell proliferation compared to a vehicle control.
Embodiment 2. A method for producing an expanded population of cochlear or vestibular cells, comprising contacting a population of cochlear supporting cells or vestibular supporting cells with:
   a) a first epigenetic agent that is a histone lysine demethylase (KDM) inhibitor and;
   b) a Wnt agonist
   wherein steps (a) and (b) can occur in any order or simultaneously, thereby producing an expanded population of cochlear or vestibular cells compared to a vehicle control.
Embodiment 3. The method of Embodiment 1 or Embodiment 2, wherein the cochlear supporting cell(s) or vestibular supporting cell(s) express(es) leucine-rich repeat-containing G-protein coupled receptor 5 (Lgr5).
Embodiment 4. The method of any preceding Embodiment, wherein the cochlear supporting cell(s) or vestibular supporting cell(s) are/is a mature cell(s).
Embodiment 5. The method of any of Embodiments 2-4, wherein the expanded population of cochlear or vestibular cells expresses leucine-rich repeat-containing G-protein coupled receptor 5 (Lgr5).
Embodiment 6. The method of any preceding Embodiment, wherein the cochlear supporting cell(s) or vestibular supporting cell(s) are/is a cochlear supporting cell(s).
Embodiment 7. The method of Embodiment 6, wherein the expanded population of cochlear or vestibular cells are cochlear cells.
Embodiment 8. The method of any preceding Embodiment, wherein the KDM inhibitor in combination with the Wnt agonist increases the Lgr5 Activity of the expanded population of cochlear or vestibular cells by a factor of at least 10, 20, 30, 40, 50, 75, 100 or 200% compared to a Wnt agonist alone or a Wnt agonist in combination with valproic acid, wherein the Lgr5 Activity is measured in a Stem Cell Proliferation Assay
Embodiment 9. A method of treating a subject who has, or is at risk of, developing an inner ear hearing or balance disorder, comprising administering to the subject:
   a) a first epigenetic agent that is a histone lysine demethylase (KDM) inhibitor; and
   b) a Wnt agonist
   wherein step (a) and (b) can occur in any order or simultaneously.
Embodiment 10. The method of Embodiment 9, wherein the subject has an inner ear hearing or balance disorder.
Embodiment 11. The method of Embodiment 9 or 10, wherein the disorder is an inner ear hearing disorder.
Embodiment 12. The method of Embodiment 9 or 10, wherein the disorder is a balance disorder
Embodiment 13. The method of any of Embodiments 9-12, wherein the inner ear hearing or balance disorder is sensorineural hearing loss.
Embodiment 14. The method of any of Embodiments 9-13, wherein the treatment results in improved auditory function when assessed by behavioural audiometry or auditory brainstem response (ABR) testing.
Embodiment 15. The method of any preceding Embodiment, wherein the KDM inhibitor is AS 8351, TC-E 5002 or EPT 103182.
Embodiment 16. The method of any preceding Embodiment, wherein the KDM inhibitor is AS 8351.
Embodiment 17. The method of any preceding Embodiment, wherein the KDM inhibitor is TC-E 5002.
Embodiment 18. The method of any preceding Embodiment, wherein the KDM inhibitor is EPT-103182.
Embodiment 19. The method of Embodiment 6, wherein the AS 8351 is at a concentration of about between 0.5 μM to 5 μM.
Embodiment 20. The method of Embodiment 17, wherein the TC-E 5002 is at a concentration of about between 0.1 μM to 10 μM.
Embodiment 21. The method of Embodiment 18, wherein the EPT-103182 is at a concentration of about 1 nM to 100 nM.
Embodiment 22. The method of any preceding Embodiment, wherein the Wnt agonist is a GSK3 inhibitor.
Embodiment 23. The method of Embodiment 22, wherein the GSK3 inhibitor is selected from the group consisting of: AZD1080, LY2090314, a substituted 3-Imidazo[1,2-a]pyridin-3-yl-4-(1,2,3,4-tetrahydro-[1,4]diazepino-[6,7,1-hi]indol-7-yl)pyrrole-2,5-dione, GSK3 inhibitor XXII or CHIR99021.
Embodiment 24. The method of Embodiment 23, wherein the GSK3 inhibitor is AZD1080.
Embodiment 25. The method of Embodiment 23, wherein the GSK3 inhibitor is LY2090314.
Embodiment 26. The method of Embodiment 23, wherein the GSK3 inhibitor is a substituted 3-Imidazo[1,2-a]pyridin-3-yl-4-(1,2,3,4-tetrahydro-[1,4]diazepino-[6,7,1-hi]indol-7-yl)pyrrole-2,5-dione.
Embodiment 27. The method of Embodiment 23, wherein the GSK3 inhibitor is GSK3 inhibitor XXII.
Embodiment 28. The method of Embodiment 23, wherein the GSK3 inhibitor is CHIR99021.
Embodiment 29. The method of Embodiment 23, wherein AZD1080 is at a concentration of about between 0.5 μM to 5 μM.
Embodiment 30. The method of Embodiment 25, wherein LY2090314 is at a concentration of about between 4 nM to 40 nM.
Embodiment 31. The method of Embodiment 26, wherein a substituted 3-Imidazo[1,2-a]pyridin-3-yl-4-(1,2,3,4-tetrahydro-[1,4]diazepino-[6,7,1-hi]indol-7-yl)pyrrole-2,5-dione is at a concentration of about between 5 nM to 500 nM.
Embodiment 32. The method of Embodiment 27, wherein GSK3 inhibitor XXII at a concentration of about between 0.1 μM to 1 μM.
Embodiment 33. The method of Embodiment 28, wherein CHIR99021 is at a concentration of about between 1 μM to 10 μM.
Embodiment 34. The method of any preceding Embodiment, further comprising contacting the cochlear or vestibular supporting cell(s) with, or administering to the subject, a second epigenetic agent.
Embodiment 35. The method of Embodiment 34, wherein the second epigenetic agent is an HDAC inhibitor, an EZH2 inhibitor, a DOT1L inhibitor, or an LSD1 inhibitor.
Embodiment 36. The method of Embodiment 35, wherein the HDAC inhibitor is Valproic Acid (VPA)
Embodiment 37. The method of Embodiment 36, wherein the VPA is at a concentration of about between 100 μM to 4,000 μM.
Embodiment 38. The method of Embodiment 35, wherein the EZH2 inhibitor an enzymatic inhibitor.
Embodiment 39. The method of Embodiment 35, wherein the EZH2 inhibitor is selected from the group consisting of: CPI-1205, E11, PF-06821497, tazemetostat, valemetostat, CPI-169, CPI-360, EPZ011989, UNC 2399, and PF 06726304.
Embodiment 40. The method of Embodiment 35, wherein the EZH2 inhibitor is CPI-1205.
Embodiment 41. The method of Embodiment 35, wherein the EZH2 inhibitor is E11.
Embodiment 42. The method of Embodiment 35, wherein the EZH2 inhibitor is PF-06821497.
Embodiment 43. The method of Embodiment 35, wherein the EZH2 inhibitor is tazemetostat.
Embodiment 44. The method of Embodiment 35, wherein the EZH2 inhibitor is valemetostat.
Embodiment 45. The method of Embodiment 35, wherein the EZH2 inhibitor is CPI-169.
Embodiment 46. The method of Embodiment 40, wherein the CPI-1205 is at a concentration of about between 10 μM to 1000 μM.
Embodiment 47. The method of Embodiment 41, wherein the E11 is at a concentration of about between 1 μM to 10 μM.
Embodiment 48. The method of Embodiment 42, wherein the PF-06821497 is at a concentration of about between 1 nM to 100 nM.
Embodiment 49. The method of Embodiment 43, wherein the tazemetostat is at a concentration of about between 0.1 μM to 1.5 μM.
Embodiment 50. The method of Embodiment 44, wherein the valemetostat is at a concentration of about between 10 nM to 1000 nM.
Embodiment 51. The method of Embodiment 45, wherein the CPI-169 is at a concentration of about between 1 μM to 10 μM.
Embodiment 52. The method of Embodiment 35, wherein the DOT1L inhibitor is a S-adenosyl methionine (SAM) competitive inhibitor.
Embodiment 53. The method of Embodiment 35, wherein the DOT1L inhibitor is selected from the group consisting of EPZ004777, pinometostat and SGC0946.
Embodiment 54. The method of Embodiment 53, wherein the DOT1L inhibitor is EPZ004777.
Embodiment 55. The method of Embodiment 53, wherein the DOT1L inhibitor is pinometostat.
Embodiment 56. The method of Embodiment 53, wherein the DOT1L inhibitor is SGC0946.
Embodiment 57. The method of Embodiment 54, wherein the EPZ004777 is at a concentration of about between 0.5 μM to 45 μM.
Embodiment 58. The method of Embodiment 55, wherein the pinometostat is at a concentration of about between 0.1 μM to 10 μM.
Embodiment 59. The method of Embodiment 56, wherein the SGC0946 is at a concentration of about between 0.5 μM to 5 μM.
Embodiment 60. The method of Embodiment 35, wherein the LSD1 inhibitor is irreversible.
Embodiment The method of Embodiment 35, wherein the LSD1 inhibitor is selected from the group consisting of GSK-2879552, GSK-LSD1, Tranylcypromine, Phenelzine sulfate, RN-1, or ORY-1001.

Embodiment 62. The method of Embodiment 35, wherein the LSD1 inhibitor is GSK2879552.
Embodiment 63. The method of Embodiment 35, wherein the LSD1 inhibitor is GSK-LSD1.
Embodiment 63. The method of Embodiment 35, wherein the LSD1 inhibitor is Tranylcypromine.
Embodiment 65. The method of Embodiment 35, wherein the LSD1 inhibitor is Phenelzine sulfate.
Embodiment 66. The method of Embodiment 62, wherein GSK2879552 is at a concentration of about between 4 nM to 30 μM.
Embodiment 67. The method of Embodiment 62, wherein GSK-LSD1 is at a concentration of about between 4 nM to 50 μM.
Embodiment 68. The method of Embodiment 63, wherein Tranylcypromine is at a concentration of about between 0.1 μM to 20 μM.
Embodiment 69. The method of Embodiment 65, wherein Phenelzine sulfate at a concentration of about between 0.1 μM to 10 μM.
Embodiment 77. The method of any preceding Embodiment, wherein the KDM inhibitor is administered locally and/or systemically.
Embodiment 78. The method of any preceding Embodiment, wherein the KDM inhibitor is administered locally.
Embodiment 79. The method of any preceding Embodiment, wherein the KDM inhibitor is administered systemically.
Embodiment 80. The method of any preceding Embodiment, wherein the KDM inhibitor is administered locally and systemically.
Embodiment 81. The method of any preceding Embodiment, wherein the Wnt agonist is administered locally and/or systemically.
Embodiment 82. The method of any preceding Embodiment, wherein the Wnt agonist is administered locally.
Embodiment 83. The method of any preceding Embodiment, wherein the Wnt agonist is administered systemically.
Embodiment 84. The method of any preceding Embodiment, wherein the Wnt agonist is administered locally and systemically.
Embodiment 85 The method of any preceding Embodiment, wherein the second epigenetic agent is administered locally and/or systemically.
Embodiment 86. The method of any preceding Embodiment, wherein the second epigenetic agent is administered locally.
Embodiment 87. The method of any preceding Embodiment, wherein the second epigenetic agent is administered systemically.
Embodiment 88. The method of any preceding Embodiment, wherein the second epigenetic agent is administered locally and systemically.
Embodiment 89. The method of any of Embodiments 77-88, wherein the local administration is to the tympanic membrane, the middle ear or the inner ear.
Embodiment 90. The method of Embodiment 85, wherein the local administration is to the middle ear
Embodiment 91. The method of any of Embodiments 77-88, wherein the systemic administration is oral or parenteral.
Embodiment 92 The method of Embodiment 91, wherein the systemic administration is oral.
Embodiment 93. The method of any of Embodiments 77-92, wherein the KDM inhibitor is AS 8351, TC-E 5002, or EPT-103182.
Embodiment 94. The method of any of Embodiments 77-92, wherein the KDM inhibitor is AS 8351 and is administered locally at a dose of 2 μM.
Embodiment 95. The method of any of Embodiments 77-92, wherein the KDM inhibitor is TC-E 5002 and is administered locally at a dose of 400 nm. 96. The method of any of Embodiments 77-92, wherein the KDM inhibitor is AS 8351 and is administered systemically at a unit dose of 100 mg.
Embodiment 97. The method of any of Embodiments 77-92, wherein the KDM inhibitor is TC-E 5002 and is administered systemically at a unit dose of 100 mg.
Embodiment 98. The method of any of Embodiments 77-97, wherein the Wnt agonist is CHIR99021 and is administered locally at a dose of 4 μM.
Embodiment 99. The method of any of Embodiments 77-97, wherein the second epigenetic agent is valproic acid (VPA) and is administered locally at a dose of 1 mM
Embodiment 100. The method of any of Embodiments 77-97, wherein the second epigenetic agent is valproic acid (VPA) and is administered systemically at a unit dose of 500 mg.
Embodiment 101. A pharmaceutical composition comprising a first epigenetic agent that is a KDM inhibitor, a Wnt agonist and a pharmaceutically acceptable carrier.
Embodiment 102. The pharmaceutical composition of Embodiment 101, wherein the KDM inhibitor is AS 8351, TC-E 5002, or EPT-103182
Embodiment 103. The pharmaceutical composition of Embodiment 102, wherein the KDM inhibitor is AS 8351.
Embodiment 104. The pharmaceutical composition of Embodiment 102, wherein the KDM inhibitor is TC-E 5002.
Embodiment 105. The pharmaceutical composition of Embodiment 102, wherein the KDM inhibitor is EPT-103182.
Embodiment 106. The pharmaceutical composition of Embodiment 103, wherein the AS 8351 is at a concentration of about between 0.5 μM to 5 μM.
Embodiment 107. The pharmaceutical composition of Embodiment 104, wherein the TC-E 5002 is at a concentration of about between 0.1 μM to 10 μM.
Embodiment 108. The pharmaceutical composition of Embodiment 104, wherein the EPT-103812 is at a concentration of about between 1 μM to 100 μM.
Embodiment 109. The pharmaceutical composition of any of Embodiments 101-108, wherein the Wnt agonist is a GSK3 inhibitor.
Embodiment 110. The pharmaceutical composition of Embodiment 109, wherein the GSK3 inhibitor is selected from the group consisting of: AZD1080, LY2090314, a substituted 3-Imidazo[1,2-a]pyridin-3-yl-4-(1,2,3,4-tetrahydro-[1,4]diazepino-[6,7,1-hi]indol-7-yl)pyrrole-2,5-dione, GSK3 inhibitor XXII or CHIR99021.
Embodiment 111. The pharmaceutical composition of Embodiment 110, wherein the GSK3 inhibitor is AZD1080.
Embodiment 112. The pharmaceutical composition of Embodiment 110, wherein the GSK3 inhibitor is LY2090314.
Embodiment 113. The pharmaceutical composition of Embodiment 110, wherein the GSK3 inhibitor is a substituted 3-Imidazo[1,2-a]pyridin-3-yl-4-(1,2,3,4-tetrahydro-[1,4]diazepino-[6,7,1-hi]indol-7-yl)pyrrole-2,5-dione.
Embodiment 114. The pharmaceutical composition of Embodiment 110, wherein the GSK3 inhibitor is GSK3 inhibitor XXII.
Embodiment 115. The pharmaceutical composition of Embodiment 110, wherein the GSK3 inhibitor is CHIR99021.
Embodiment 116. The pharmaceutical composition of Embodiment 111, wherein AZD1080 is at a concentration of about between 0.5 mM to 5 mM.
Embodiment 117. The pharmaceutical composition of Embodiment 112, wherein LY2090314 is at a concentration of about between 4 μM to 40 μM.
Embodiment 118. The pharmaceutical composition of Embodiment 113, wherein a substituted 3-Imidazo[1,2-a]pyridin-3-yl-4-(1,2,3,4-tetrahydro-[1,4]diazepino-[6,7,1-hi]indol-7-yl)pyrrole-2,5-dione is at a concentration of about between 5 μM to 500 μM.
Embodiment 119. The pharmaceutical composition of Embodiment 114, wherein GSK3 inhibitor XXII at a concentration of about between 0.1 mM to 1 mM.
Embodiment 120. The pharmaceutical composition of Embodiment 115, wherein CHIR99021 is at a concentration of about between 1 mM to 10 mM.
Embodiment 121. The pharmaceutical composition of any of Embodiments 101-120 further comprising further comprising a second epigenetic agent.
Embodiment 123. The pharmaceutical composition of Embodiment 122, wherein the HDAC inhibitor is Valproic Acid (VPA)
Embodiment 124. The pharmaceutical composition of Embodiment 123, wherein the VPA is at a concentration of about between 100 mM to 4,000 mM.
Embodiment 125. The pharmaceutical composition of Embodiment 122, wherein the EZH2 inhibitor an enzymatic inhibitor.
Embodiment 126. The pharmaceutical composition of Embodiment 122, wherein the EZH2 inhibitor is selected from the group consisting of: CPI-1205, E11, PF-06821497, tazemetostat, valemetostat, CPI-169, CPI-360, EPZ011989, UNC 2399, and PF 06726304.
Embodiment 127. The pharmaceutical composition of Embodiment 126, wherein the EZH2 inhibitor is CPI-1705.
Embodiment 128. The pharmaceutical composition of Embodiment 126, wherein the EZH2 inhibitor is E11.
Embodiment 129. The pharmaceutical composition of Embodiment 126, wherein the EZH2 inhibitor is PF-06821497.
Embodiment 130. The pharmaceutical composition of Embodiment 126, wherein the EZH2 inhibitor is tazemetostat.
Embodiment 131. The pharmaceutical composition of Embodiment 126, wherein the EZH2 inhibitor is valemetostat.
Embodiment 132. The pharmaceutical composition of Embodiment 126, wherein the EZH2 inhibitor is CPI-169.
Embodiment 133. The pharmaceutical composition of Embodiment 127, wherein the CPI-1205 is at a concentration of about between 1 mM to 10 mM.
Embodiment 134. The pharmaceutical composition of Embodiment 128, wherein the E11 is at a concentration of about between 10 μM to 1000 μM.
Embodiment 135. The pharmaceutical composition of Embodiment 129, wherein the PF-06821497 is at a concentration of about between 1 μM to 100 μM.
Embodiment 136. The pharmaceutical composition of Embodiment 130, wherein the tazemetostat is at a concentration of about between 0.1 mM to 10 mM.
Embodiment 137. The pharmaceutical composition of Embodiment 131, wherein the valemetostat is at a concentration of about between 10 μM to 1000 μM.
Embodiment 138. The pharmaceutical composition of Embodiment 132, wherein the CPI-169 is at a concentration of about between 1 mM to 10 MM.
Embodiment 139. The pharmaceutical composition of Embodiment 122, wherein the DOT1L inhibitor is a S-adenosyl methionine (SAM) competitive inhibitor.
Embodiment 140. The pharmaceutical composition of Embodiment 122, wherein the DOT1L inhibitor is selected from the group consisting of EPZ004777, pinometostat and SGC0946.
Embodiment 141. The pharmaceutical composition of Embodiment 140, wherein the DOT1L inhibitor is EPZ004777.
Embodiment 142. The pharmaceutical composition of Embodiment 140, wherein the DOT1L inhibitor is pinometostat.
Embodiment 143. The pharmaceutical composition of Embodiment 140, wherein the DOT1L inhibitor is SGC0946.
Embodiment 144. The pharmaceutical composition of Embodiment 141, wherein the is EPZ004777 is at a concentration of about between 0.5 mM to 45 mM.
Embodiment 145. The pharmaceutical composition of Embodiment 142, wherein the pinometostat is at a concentration of about between 0.1 mM to 10 mM.
Embodiment 146. The pharmaceutical composition of Embodiment 143, wherein the SGC0946 is at a concentration of about between 0.5 mM to 5 mM.
Embodiment 147. The pharmaceutical composition of Embodiment 122, wherein the LSD1 inhibitor is irreversible.
Embodiment 148. The pharmaceutical composition of Embodiment 122, wherein the LSD1 inhibitor is selected from the group consisting of GSK-2879552, GSK-LSD1, Tranylcypromine, Phenelzine sulfate, RN-1, or ORY-1001.
Embodiment 149. The pharmaceutical composition of Embodiment 148, wherein the LSD1 inhibitor is GSK2879552.
Embodiment 150. The pharmaceutical composition of Embodiment 148, wherein the LSD1 inhibitor is GSK-LSD1.
Embodiment 151. The pharmaceutical composition of Embodiment 148, wherein the LSD1 inhibitor is Tranylcypromine.
Embodiment 152. The pharmaceutical composition of Embodiment 148, wherein the LSD1 inhibitor is Phenelzine sulfate.
Embodiment 153. The pharmaceutical composition of Embodiment 149, wherein GSK2879552 is at a concentration of about between 4 nM to 30 μM.
Embodiment 154. The pharmaceutical composition of Embodiment 150, wherein GSK-LSD1 is at a concentration of about between 4 nM to 50 μM.
Embodiment 155. The pharmaceutical composition of Embodiment 151, wherein Tranylcypromine is at a concentration of about between 0.1 μM to 20 μM.
Embodiment 156. The pharmaceutical composition of Embodiment 152, wherein Phenelzine sulfate at a concentration of about between 0.1 μM to 10 μM.
Embodiment 164. The pharmaceutical composition of any of Embodiments 101-163, wherein the pharmaceutical composition is in a biocompatible matrix.
Embodiment 165. The pharmaceutical composition of Embodiment 164, wherein the biocompatible matrix comprises hyaluronic acid, hyaluronates, lecithin gels, pluronics, poly(ethyleneglycol), poloxamers, chitosans, xyloglucans, collagens, fibrins, polyesters, poly(lactides), poly(glycolide), poly(lactic-co-glycolic acid (PLGA), sucrose acetate isobutyrate, glycerol monooleate, poly anhydrides, poly caprolactone sucrose, glycerol monooleate, silk materials, or a combination thereof.
Embodiment 166. The pharmaceutical composition of any of Embodiments 101-165, wherein the pharmaceutical composition is formulated for administration as defined in any of Embodiments 73-94
Embodiment 167. The pharmaceutical composition any of Embodiments 101-166, for use in treating or preventing an inner ear hearing or balance disorder.
Embodiment 168. The pharmaceutical composition for use according to Embodiment 167, wherein the inner ear hearing or balance disorder is sensorineural hearing loss.
Embodiment 169. Use of the pharmaceutical composition of any of Embodiments 101-168 in the manufacture of a medicament for the treatment or prevention of an inner ear hearing or balance disorder.
Embodiment 170. Use of the pharmaceutical composition according to Embodiment 169, wherein the inner ear hearing or balance disorder is sensorineural hearing loss.
Embodiment 171. A histone lysine demethylase (KDM) inhibitor for use in treating or preventing an inner ear hearing or balance disorder in a subject, wherein the subject has been, or will be, administered a Wnt agonist.
Embodiment 172. A Wnt agonist for use in treating or preventing an inner ear hearing or balance disorder in a subject, wherein the subject has been, or will be, administered a histone lysine demethylase (KDM) inhibitor.
Embodiment 173. An epigenetic agent for use in treating or preventing an inner ear hearing or balance disorder in a subject, wherein the subject has been, or will be, administered a histone lysine demethylase (KDM) inhibitor and a Wnt agonist.
Embodiment 174. The KDM inhibitor, Wnt agonist or epigenetic agent for use according to any of Embodiments 169-173, wherein the inner ear hearing or balance disorder is sensorineural hearing loss.
Embodiment 175. The KDM inhibitor, Wnt agonist or epigenetic agent for use according to any of Embodiments 169-174, wherein the treatment is as defined in any of Embodiments 9-95.
Embodiment 176. A container comprising a histone lysine demethylase (KDM) inhibitor and instructions, where those instructions describe the KDM inhibitor's use for treating or preventing an inner ear hearing or balance disorder in a subject, wherein the instructions require that the subject has been, or will be, administered a Wnt agonist.
Embodiment 177. A container comprising a Wnt agonist and instructions, where those instructions describe the Wnt agonist's use in treating or preventing an inner ear hearing or balance disorder in a subject, wherein the instructions require that the subject has been, or will be, administered a historic lysine demethylase (KDM) inhibitor.
Embodiment 178. A container comprising an epigenetic agent and instructions, where those instructions describe the epigenetic agent's use in treating or preventing an inner ear hearing or balance disorder in a subject, wherein the instructions require that the subject has been, or will be, administered a histone lysine demethylase (KDM) inhibitor and a Wnt agonist.
Embodiment 179. The container according to any of Embodiments 176-178, wherein the inner ear hearing or balance disorder is sensorineural hearing loss.
Embodiment 180. The container according to any of Embodiments 176-178, wherein the treatment is as defined in any of Embodiments 9-100.

The subject-matter detailed in numbered embodiments 1-2677 on pages of 173 to 355 of U.S. 62/803,347 also forms part of the invention.

The subject-matter detailed in numbered embodiments 1-2696 on pages of 209 to 389 of U.S. 62/803,351 also forms part of the invention.

The subject-matter detailed in numbered embodiments 1-2697 on pages of 172 to 292 of U.S. 62/803,352 also forms part of the invention.

The subject-matter detailed in numbered embodiments 1-2689 on pages of 174 to 354 of U.S. 62/803,353 also forms part of the invention.

### EXAMPLES

### Example 1: Materials and Methods

Mice for Cell Screening

Neonatal Lgr5-EGFP-IRES-Cre-ER mice (The Jackson Laboratory, strain 8875) were used to analyze the effects of small molecules on cochlear stem cell expansion (see Barker et al., Nature 449, 1003-7 (2007). This strain allowed for visualization and quantification of EGFP cells.

Cell Assays

All animal studies were conducted under an approved institutional protocol per National Institutes of Health guidelines. Using neonatal animals, cochleae were dissected and the organ of Corti (sensory epithelium) was separated from the stria vascularis (ion transport epithelium) and the modiolus (nerve tissue). Epithelia were then collected and treated with TrypLE for 15-20 minutes to obtain single cells. The cells were then filtered (40 mm) and suspended in a Matrigel (Corning) dome for 3D culture seeded at 0.5 cochlea per well.

Expansion of Lgr5 Cells: Cells were cultured in a 3D system and bathed in a serum free 1:1 mixture of DMEM and F12, supplemented with Glutamax (GIBCO), N2, B27 (Invitrogen), EGF (50 ng/mL; Chemicon), bFGF (50 ng/mL, Chemicon), IGF-1 (50 ng/mL; Chemicon), and small molecules for seven days. Media was changed every other day. Treatments were run in triplicate or quadruplicate.

Quantification of Cell Proliferation: Lgr5 cells were quantified after 7-10 days. Cell colonies were dissociated into single cells using TrypLE. The cells were then stained with propidium iodide (PI) and analyzed using a flow cytometer to count Lgr5-EGFP cells. The percentage of viable Lgr5 cells was plotted against the concentration in GraphPad Prism.

Quantification of Cell Proliferation, Expansion and Enrichment

Organs of Corti are dissected from Lgr5 GFP+mice and dissociated as single cells as described above. Background media contains the same supplements and growth factors at the same concentrations as described above. Assays for image quantification are run in 96 well black plates with clear bottom with cells embedded in 50% Matrigel at cell density of 500 k cells/mL with 50 uL applied to each well. Cells are cultured for 7 days, with media change every 3-4 days. After 7 days of exposure to experimental conditions (e.g. small molecules), media is then removed from culture and replaced with media containing Hoescht at a 1:2000 dilution for a final concentration of 5 ug/mL (200 uL/well). The plate is then placed in a cell culture incubator at 37 C for 1 hr. The media containing Hoescht is then removed and 200 uL/well of Cell Recovery Solution is added. The plate is then incubated on a plastic-wrapped (e.g. Saran wrap) CoolRack™ on ice for 80 minutes. Next, the plate is centrifuged for 5 minutes at 2300 RPMs (Beckman Coulter Allegra 6R centrifuge; GH 3.8 A plate rotor; ambient temperature). Cells are then imaged on Celigo using 3 channels for brightfield, blue (Hoescht), and green (Lgr5 GFP). Proliferated cell colonies are captured as summed objects in the blue channel and the green channel. The green Lgr5 GFP+cell colonies are quantified for total GFP(+) cell area, while the blue Hoescht stained colonies are quantified as total cell area. The % GFP(+) Cell Area is calculated using the total GFP(+) cell area divided by the total cell area multiplied by 100. All results are compiled and utilized to determine the effects of experimental conditions (e.g. small molecules) on the expansion and enrichment of the Lgr5 cell population.

Lateral Canal Sampling

Animals were initially anesthetized with 100 mg/kg sodium thiobutabarbital (Inactin, Sigma, St Louis, Mo.) and maintained on 0.8 to 1.2% isofluorane in oxygen. Animals were mechanically ventilated through a tracheal cannula. Tidal volume was set to maintain a 5% end-tidal CO₂ level. Heart rate and blood oxygen saturation were monitored with a pulse-oximeter (Surgivet. Waukesha, Wis.). Body temperature was maintained near 38° C. with a thermistor-controlled heating pad.

Access to the LSCC was obtained with a post-auricular incision and a lateral opening in the auditory bulla. To prepare the LSCC for injection and sampling, the bone over the canal was thinned with a dental burr, where necessary removing a branch of the facial nerve that in some animals runs parallel to the LSCC for a short distance. When the canal was visible through the thinned bone, a layer of thin cyanoacrylate glue was applied to the dry bone followed by layers of two-part silicone adhesive (Kwik-Cast, World Precision Instruments, Sarasota, Fla.). The silicone was applied thinly over the canal but multiple layers were built up at the periphery to form a hydrophobic cup structure. A 30-40 μm fenestration into the canal wall was made through the adhesives and bone using a 30° House stapes pick (N1705 80, Bausch and Lomb Inc.). The pick was sharp at the tip, but rapidly widened so that entry into the canal, and potential damage to the endolymphatic system, was minimized.

At times varied from 15 minutes to 4 hours after the end of injection, multiple perilymph samples were taken from the LSCC. The injection pipette was first removed and the drop of cyanoacrylate glue that sealed it in place was broken up with the pick, taking care to leave the silicone cup intact. The fenestration was widened to 50-70 μm to allow perilymph leakage and the emerging perilymph was collected in blunt-tipped capillaries (#53432-706, 5 μL, VWR International, Radnor, Pa.). Each capillary was marked at a nominal volume of 1 μL. Sixteen to twenty individual 1 μL perilymph samples were collected sequentially, over a 20-30 min time period. The length of each sample was immediately measured with a calibrated dissecting microscope. Samples were expelled into diluent (25 uL of 50:50 acetonitrile), with pairs of samples pooled, resulting in 8-10 measurements each. All data are presented as the 8-10 measured samples from each experiment. Analysis of compound concentration was determined by LCMS

Apical Sampling

Gradients of drug along the perilymphatic spaces were measured directly from multiple samples obtained by a technique called “sequential sampling”. When the apex is perforated, perilymph is driven out by cerebrospinal fluid (CSF) entering the basal turn of ST through the cochlear aqueduct, pushing perilymph in an apical direction along the scala. The first sample collected originates from perilymph near the apex and each following sample from perilymph that originated from a scala location progressively closer to the base. After all ST perilymph has been pushed out, subsequent samples contain CSF that has passed through the scala. Samples collected in this manner allow drug gradients along the length of ST to be quantified. Perilymph was collected from the cochlear apex as a series of individual 1 μL samples collected over a 10-20 min period. To prepare the cochlea for sample collection the middle ear mucosa overlying the cochlear apex was first removed and the bone was allowed to dry. A thin layer of cyanoacrylate glue (Permabond 101; Permabond, Pottstown, Pa.) was applied to the dry bone, followed by layers of two-part silicone adhesive (Kwik-Cast, World Precision instruments, Sarasota, Fla.), built up at the edges to form a hydrophobic cup. At the time of sampling a 30-40 μm fenestration was made at the apex through the adhesives using a 30° House stapes pick (N1705 80, Bausch and Lomb Inc.). Clear, uncontaminated fluid flows from the fenestration, accumulating on the hydrophobic surface. Fluid was collected with hand-held, blunt tipped capillary tubes (VWR 53432-706; VWR Radnor, Pa.), each marked for a nominal volume of 1 μL and taking 1-2 min to collect. The length of each sample in its capillary tube was measured with a calibrated dissecting microscope, from which the exact sample volume was established. Ten individual samples were collected in this manner, with the first sample representing the apex and each subsequent sample representing further towards the base and eventually the CSF. Samples were expelled into diluent (25 uL of 50:50 acetonitrile) and analysis of compound concentration was determined by LCMS

Example 2: LSD1 Inhibition does not Promote the Expansion of Cochlear Progenitor Cells

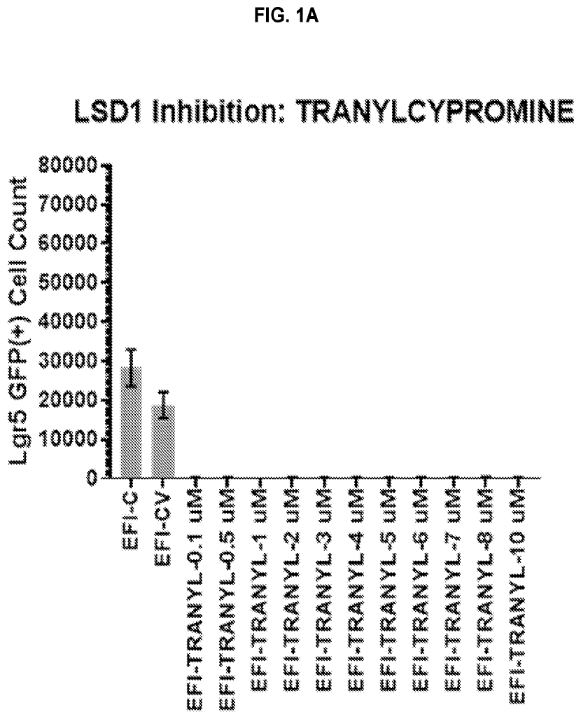

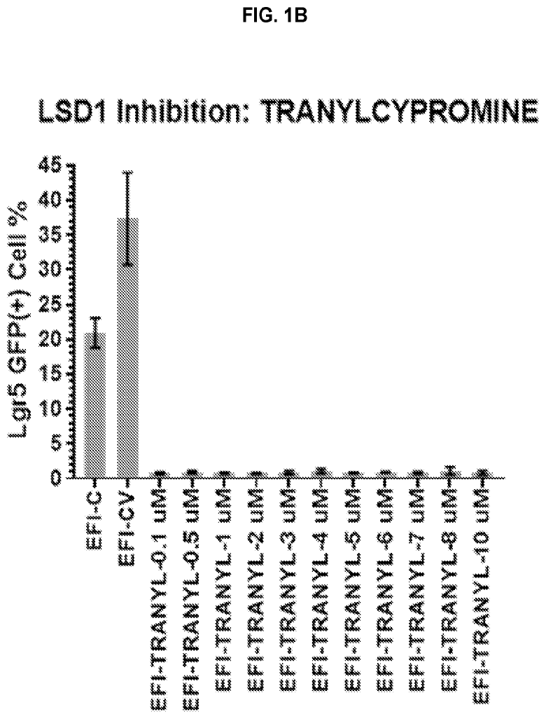

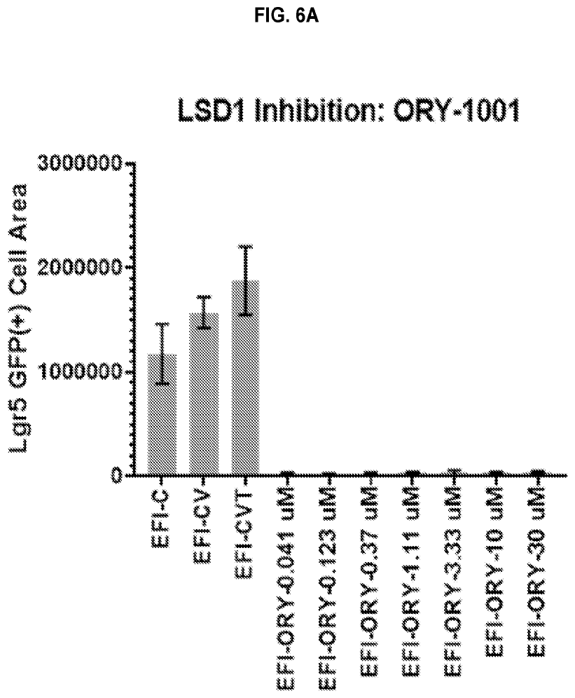

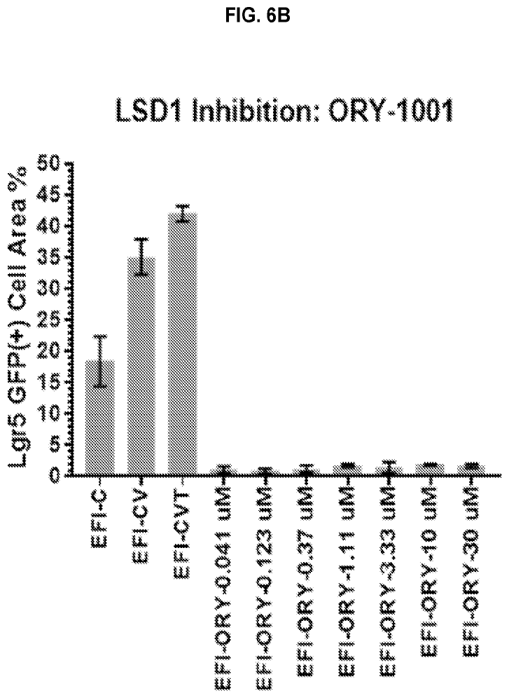

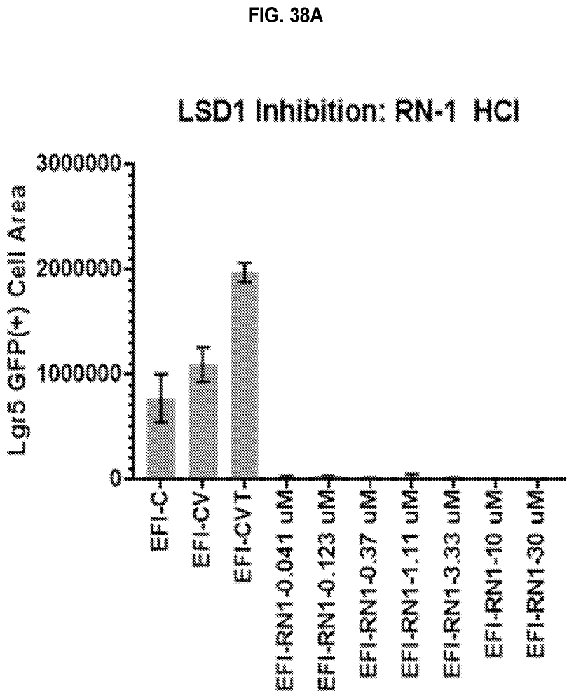

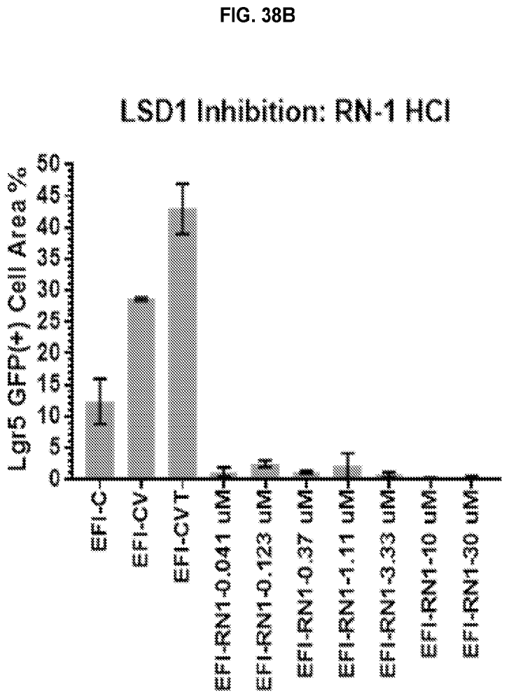

Cellular assays were carried about as described in Example 1 to determine the effect of LSD1 inhibition alone on cochlear progenitor cell proliferation and enrichment. As shown in FIG. 1A and FIG. 1B, LSD1 inhibition with tranylcypromine does not promote the proliferation or enrichment of cochlear progenitor cells. FIG. 6A and FIG. 6B show similar results, whereby LSD1 inhibition by ORY-1001 does not promote the proliferation or enrichment of cochlear progenitor cells. FIG. 38A and FIG. 38B show similar results, whereby LSD1 inhibition by RN-1 HCl does not promote the proliferation or enrichment of cochlear progenitor cells. Taken together, the results indicate that LSD1 inhibition alone does not promote proliferation or enrichment of cochlear progenitor cells.

### Example 3: LSD1 Inhibition in Combination with a Wnt Agonist Enhances the Expansion of Cochlear Progenitor Cells

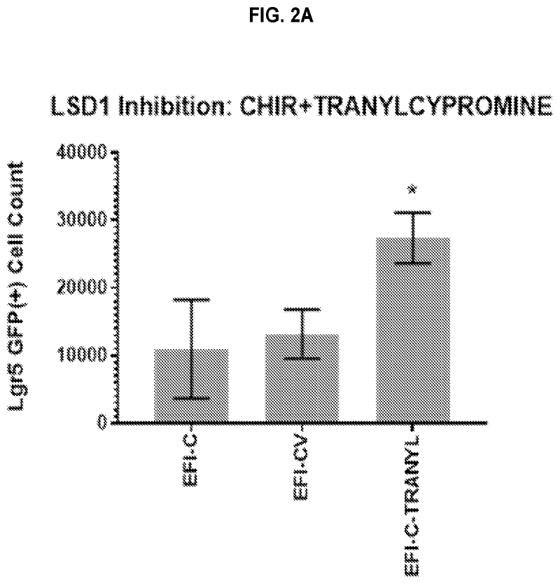

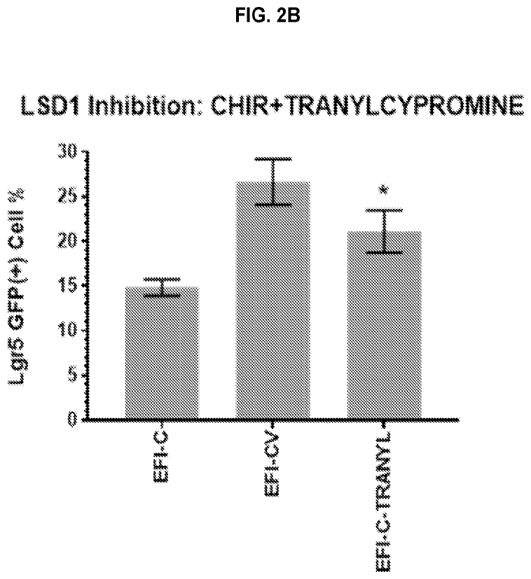

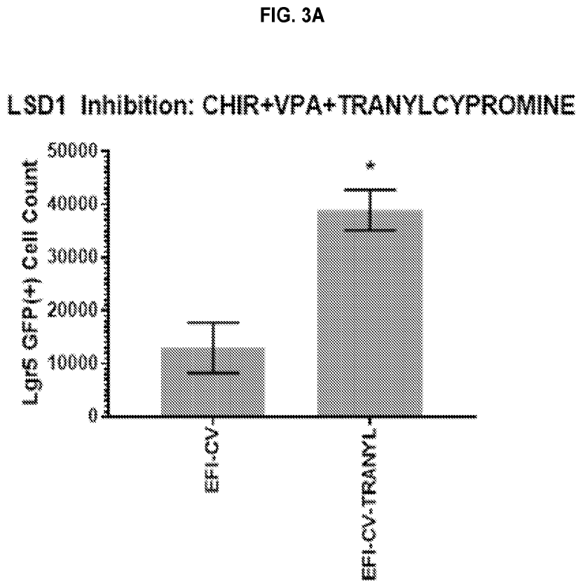

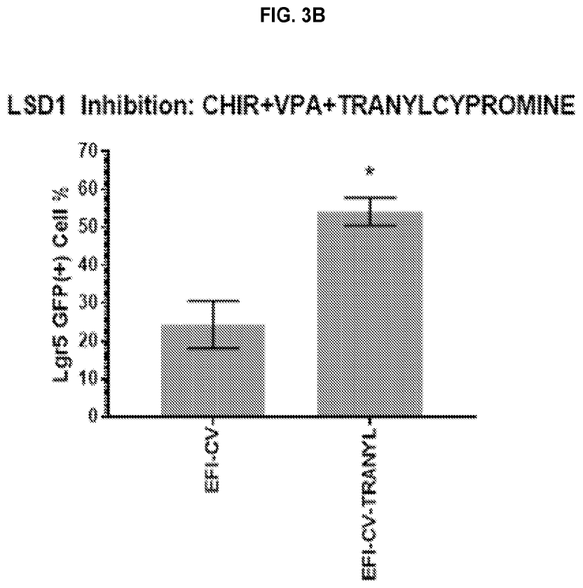

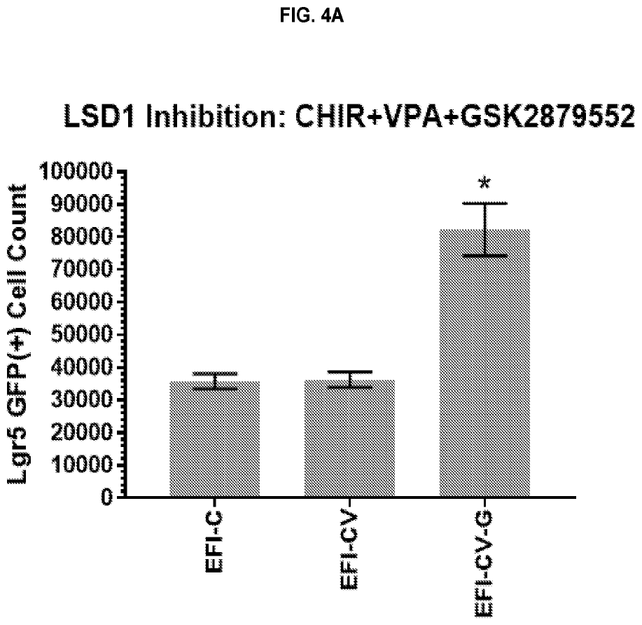

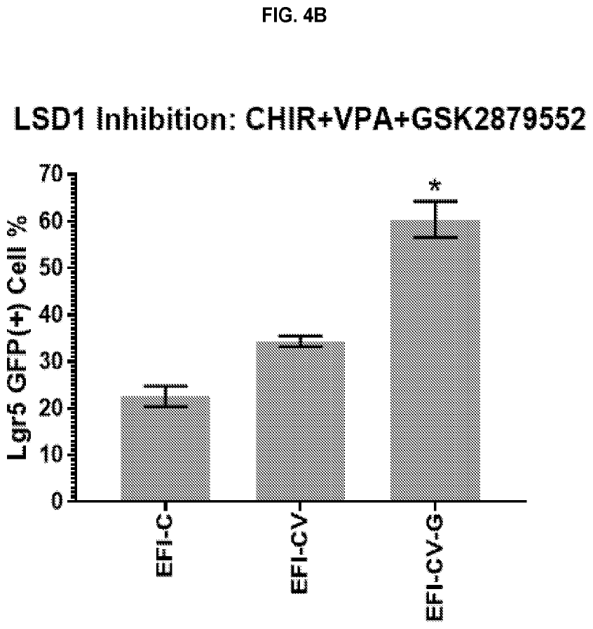

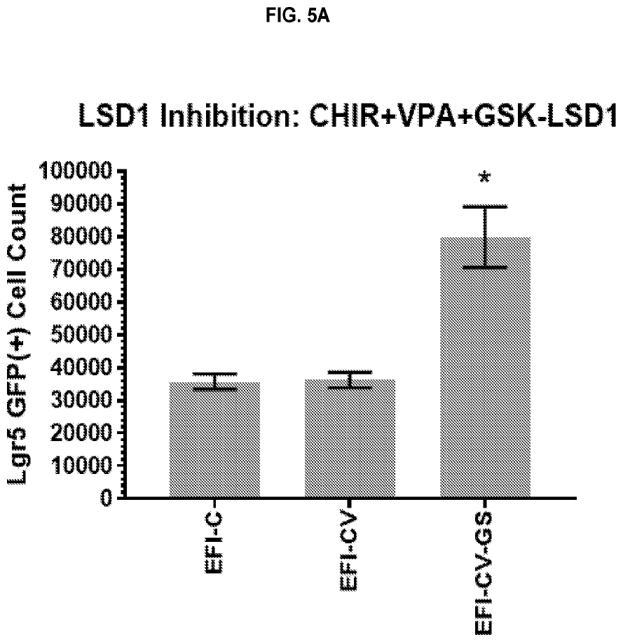

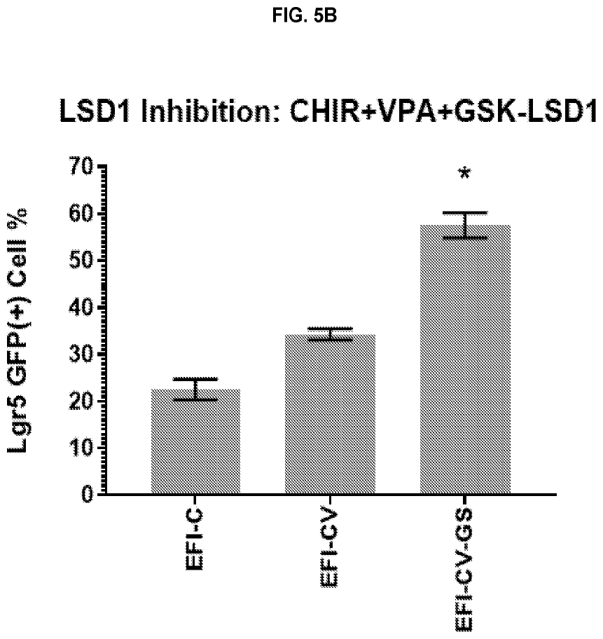

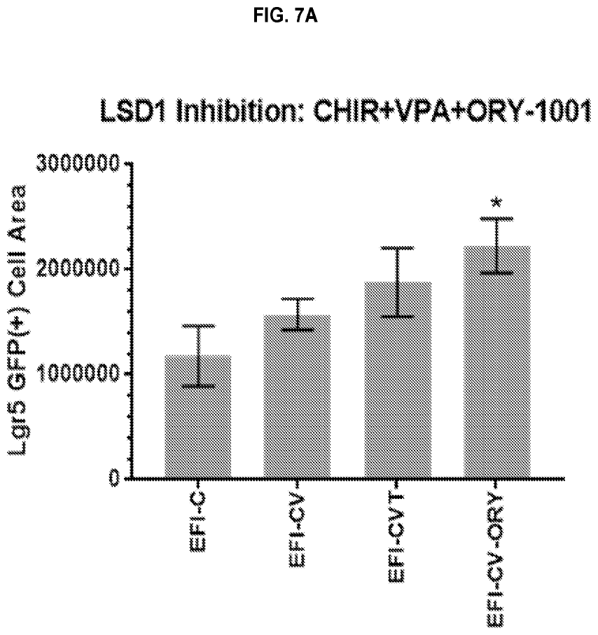

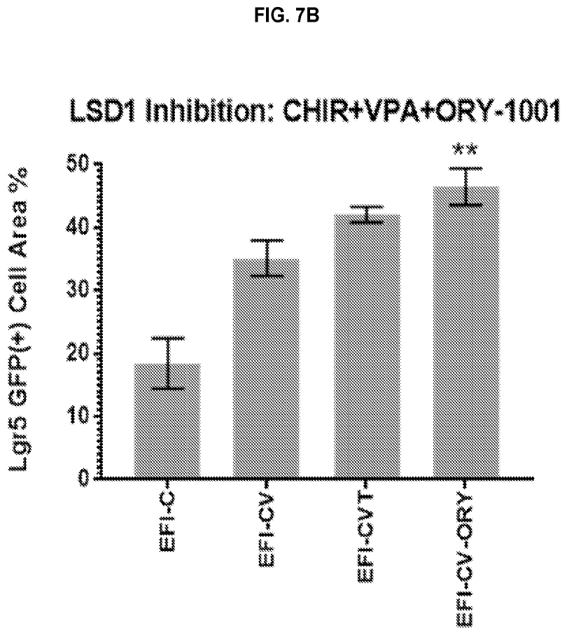

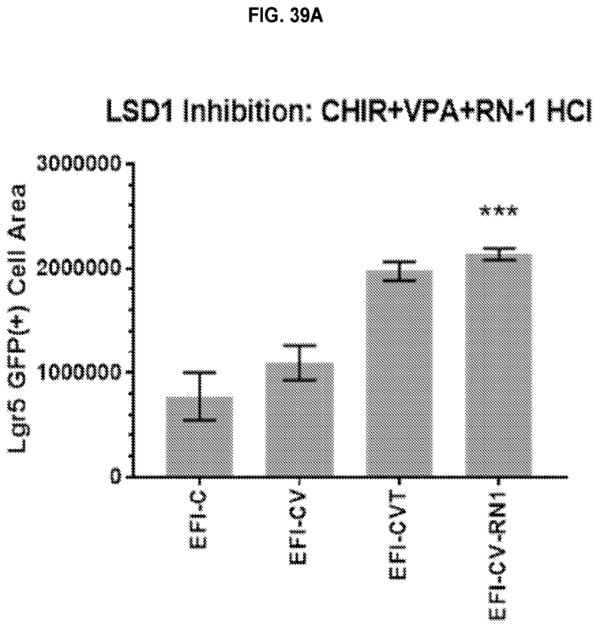

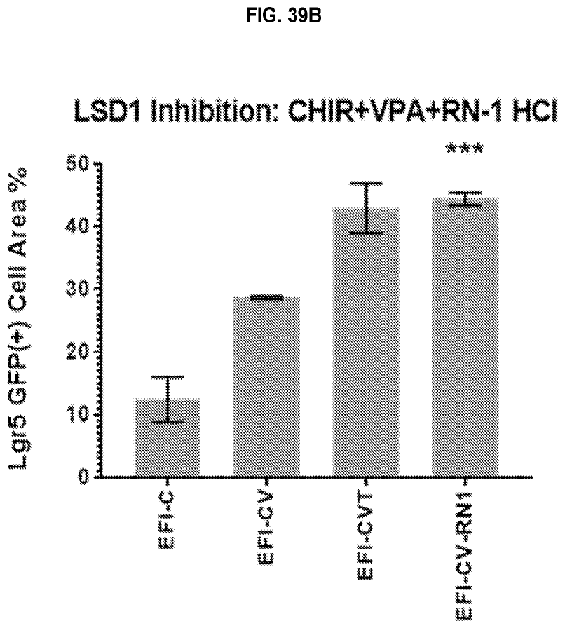

Cellular assays were carried about as described in Example 1 to determine the effect of LSD1 inhibition in combination with a Wnt agonist and Valproic Acid (VPA). As shown in FIG. 2A and FIG. 2B, Lgr5+ progenitor cells proliferate and are enriched when LSD1 inhibitor tranylcypromine is combined with Wnt agonist CHIR99021 (4 μM). As shown in FIG. FIG. 3A and FIG. 3B, Lgr5+ progenitor cells proliferate and are enriched when LSD1 inhibitor tranylcypromine is combined with Wnt agonist CHIR99021 (4 μM) and VPA (1 mM). Moreover, FIG. 4A and FIG. 4B show similar results; Lgr5+ progenitor cell proliferation and enrichment are enhanced in the presence of LSD1 inhibitor GSK2879552 (370 nM), Wnt agonist CHIR99021 (4 μM), and VPA (1 mM). FIG. 5A and FIG. 5B show similar enhancement of Lgr5+ progenitor cell proliferation and enrichment when treated with LSD1 inhibitor GSK-LSD1 (4.5 nM), Wnt agonist CHIR99021 (4 μM), and VPA (1 mM). FIG. 7A and FIG. 7B show enhanced Lgr5+ progenitor cell proliferation and enrichment achieved with LSD1 inhibitor ORY-1001 (41 nM), CHIR99021 (4 μM) and VPA (1 mM). FIG. 39A and FIG. 39B show similar results on Lgr5+ progenitor cell proliferation and enrichment achieved with LSD1 inhibitor, RN-1 HCl (41 nM), CHIR99021 (4 μM) and VPA (1 mM). Taken together the results indicate that LSD1 inhibition by a panel of inhibitors in combination with a Wnt agonist and VPA enhance Lgr5+ cochlear progenitor cell proliferation and erichment.

### Example 4: EZH2 Inhibition does not Promote the Expansion of Cochlear Progenitor Cells

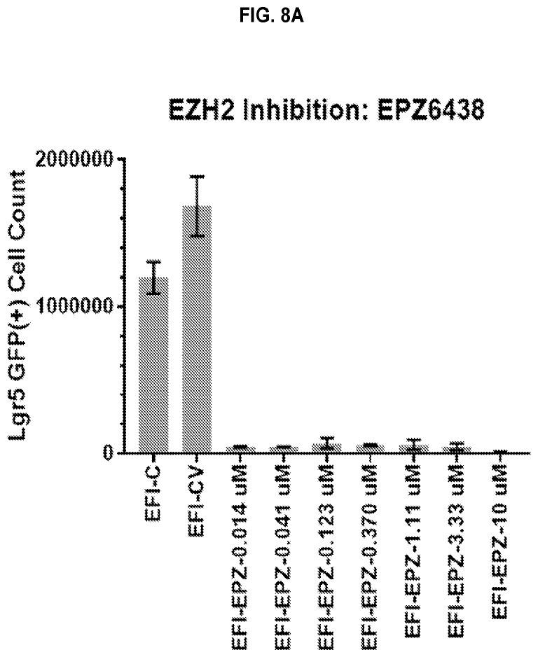

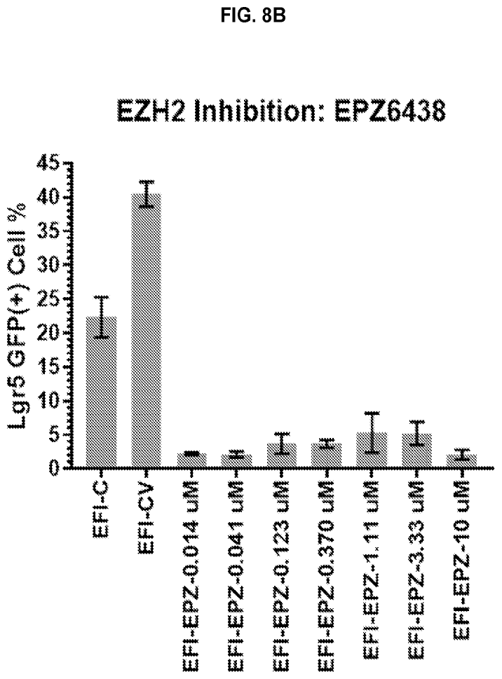

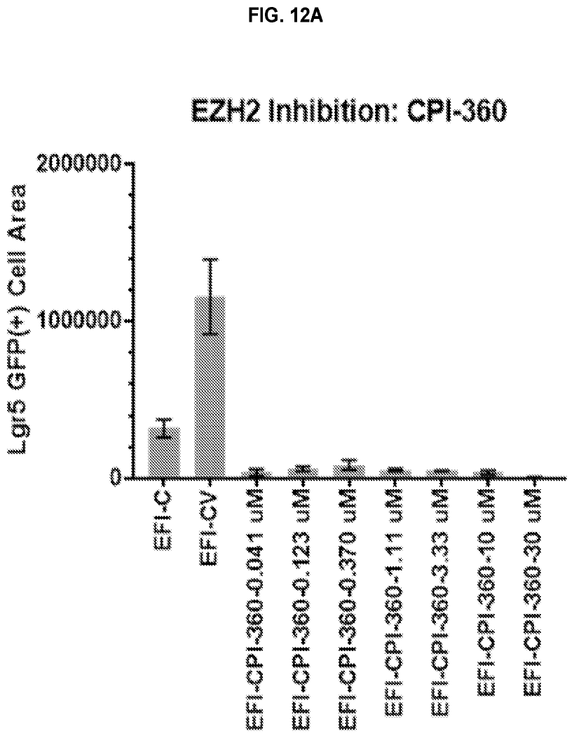

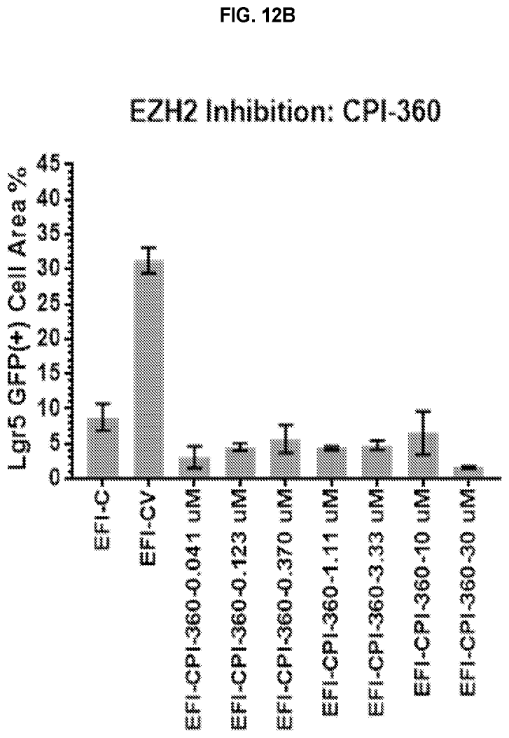

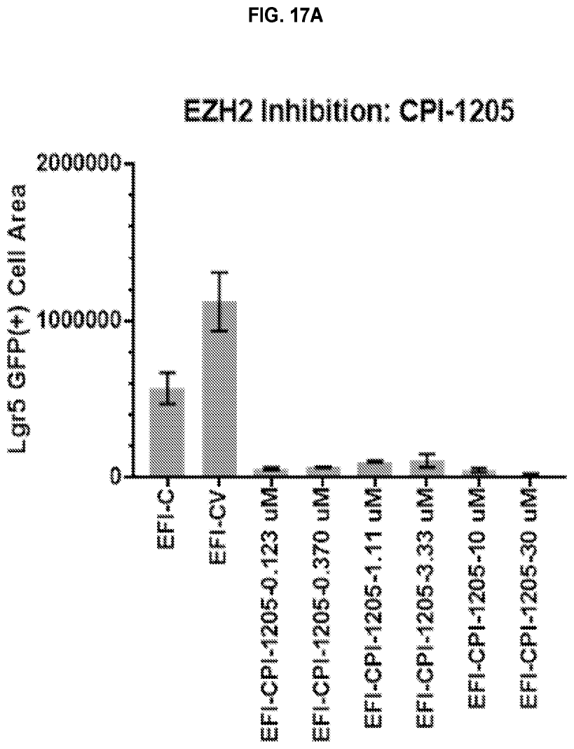

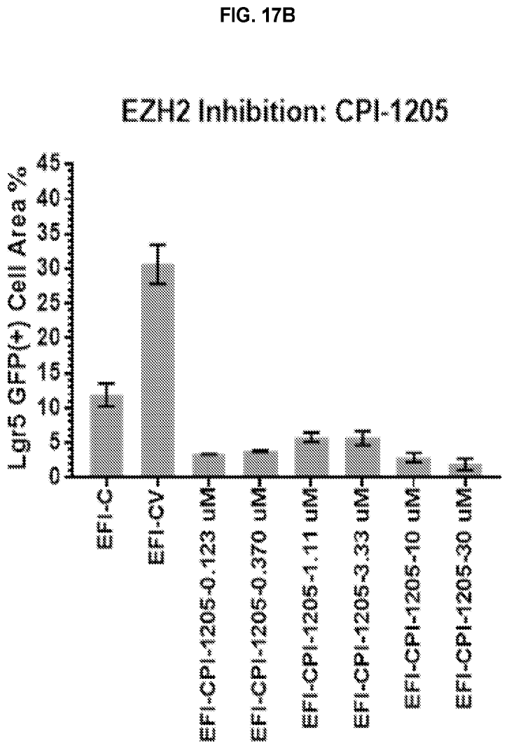

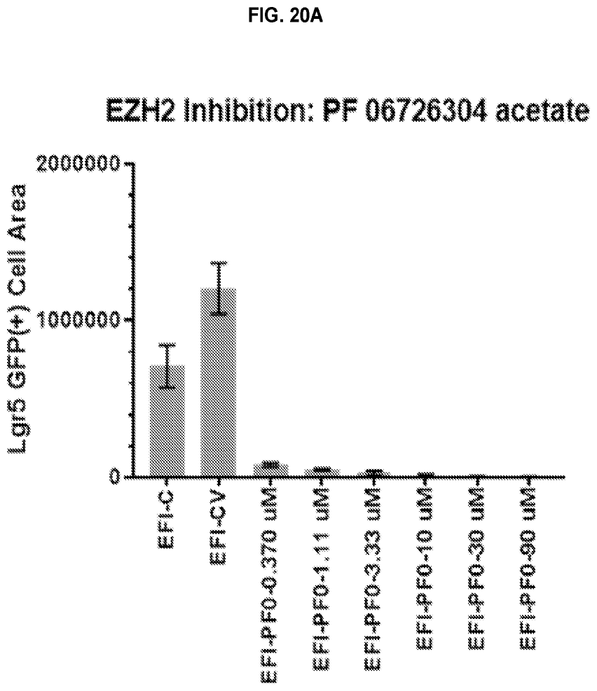

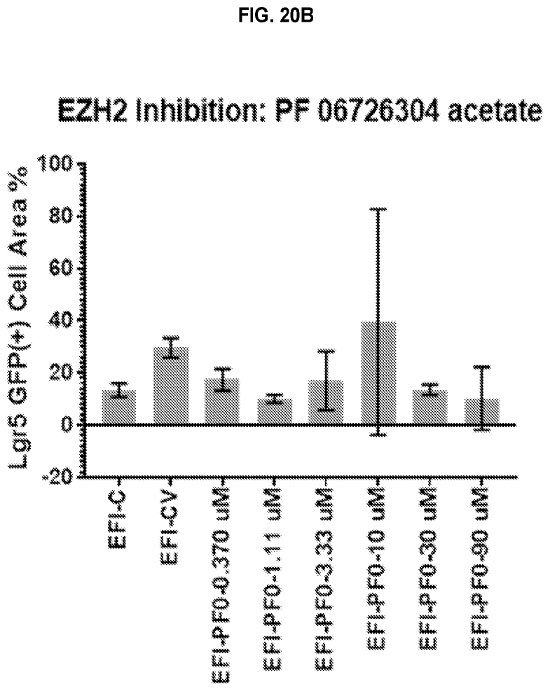

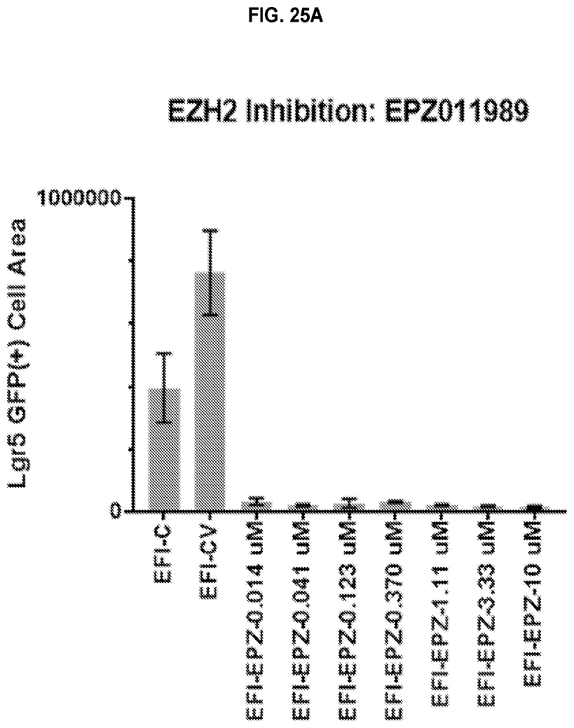

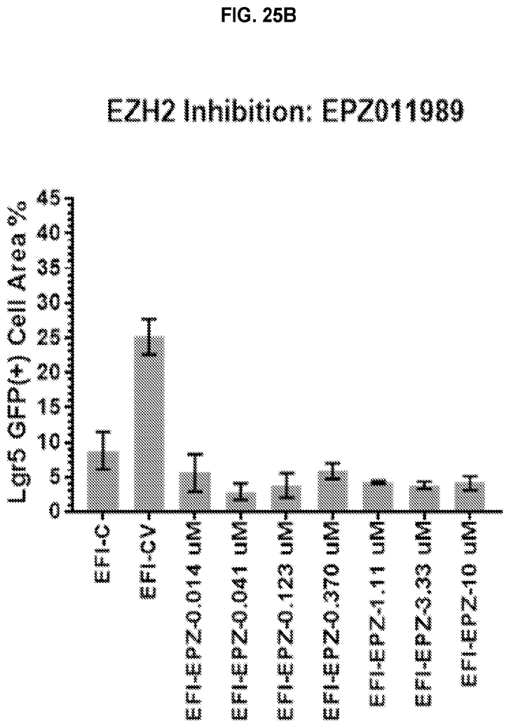

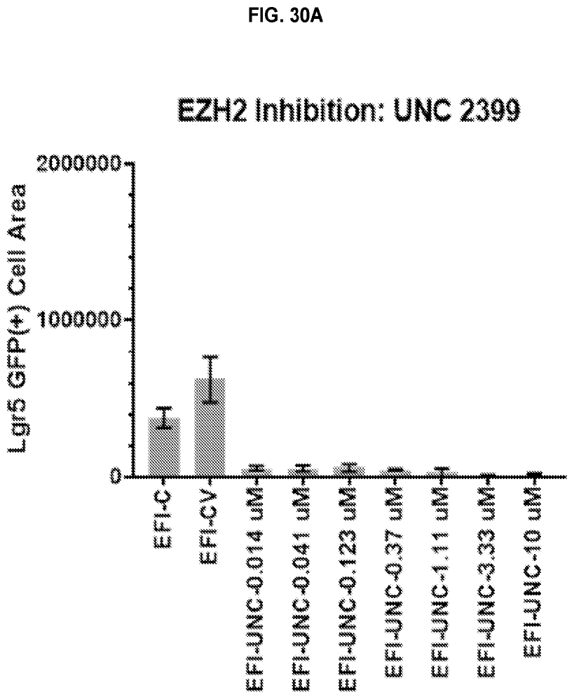

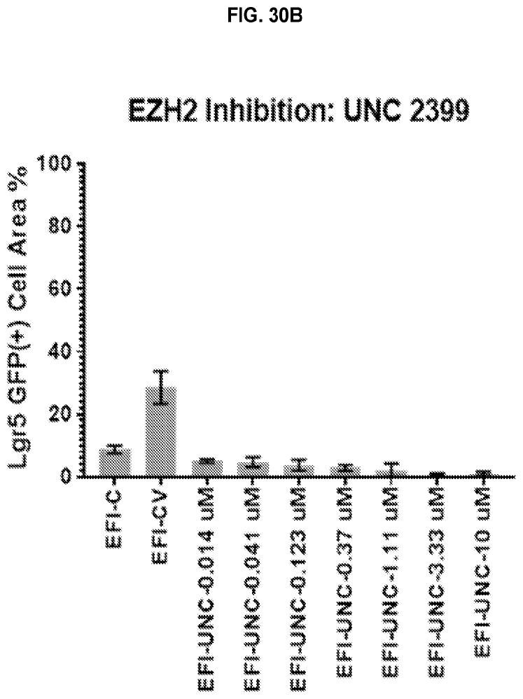

Cellular assays were carried about as described in Example 1 to determine the effect of EZH2 inhibition alone on the expansion of cochlear progenitor cells. As shown in FIG. 8A and FIG. 8B, EZH2 inhibition by EPZ6438 does not promote the proliferation or enrichment of cochlear progenitor cells. FIG. 12A and FIG. 12B show similar results, whereby EZH2 inhibition by CPI-360 does not promote the proliferation or enrichment of cochlear progenitor cells. Similarly, FIG. 17A and FIG. 17B show that EZH2 inhibition by CPI-1205 does not promote the proliferation or enrichment of cochlear progenitor cells. FIG. 20A and FIG. 20B shows EZH2 inhibition by PF 06726304 acetate does not promote the proliferation or enrichment of cochlear progenitor cells. Moreover, FIG. 25A and FIG. 25B show EZH2 inhibition by EPZ011989 does not promote proliferation or enrichment of cochlear progenitor cells. Likewise, FIG. 30A and FIG. 30B show EZH2 inhibition by UNC 2399 does not promote the proliferation or enrichment of cochlear progenitor cells. Taken together, the results indicate that EZH2 inhibition alone does not promote expansion or enrichment of cochlear progenitor cells.

### Example 5: EZH2 Inhibition in Combination with a Wnt Agonist Enhances the Expansion of Cochlear Progenitor Cells

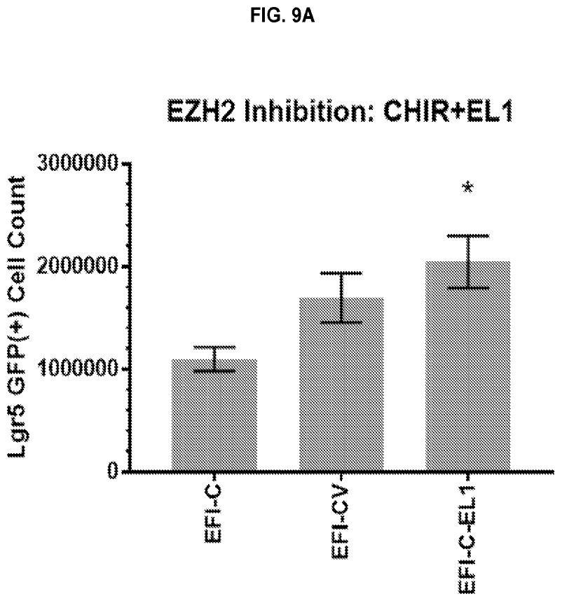

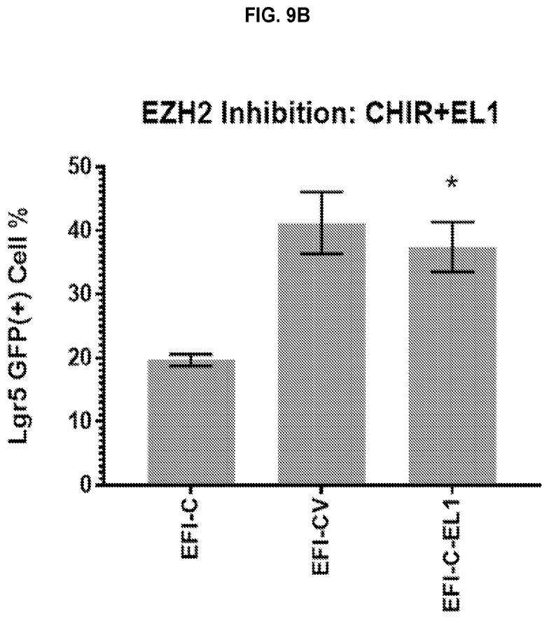

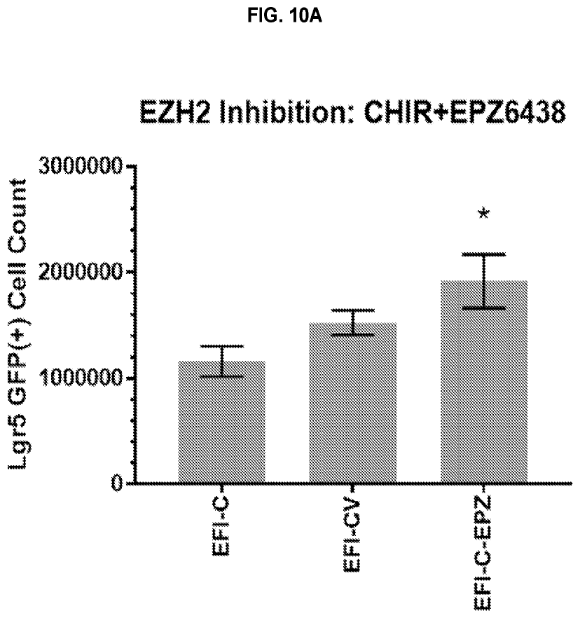

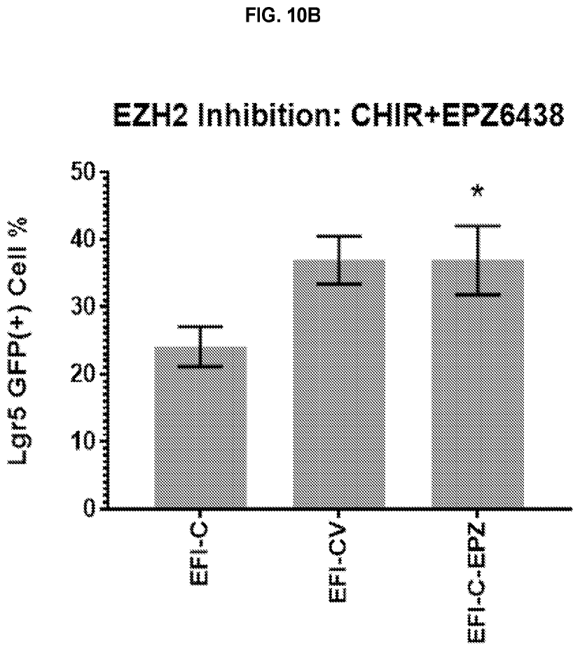

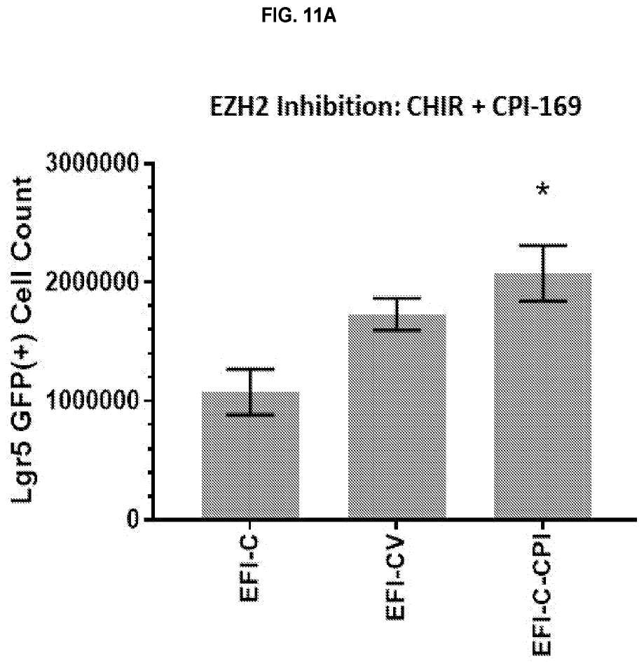

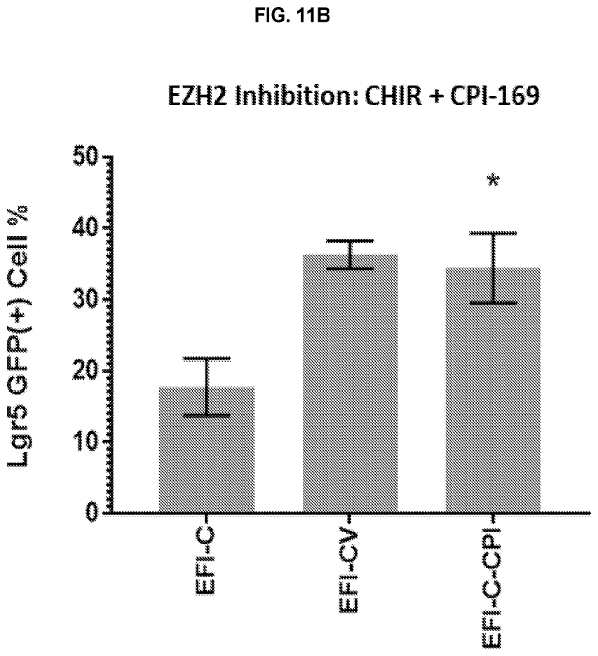

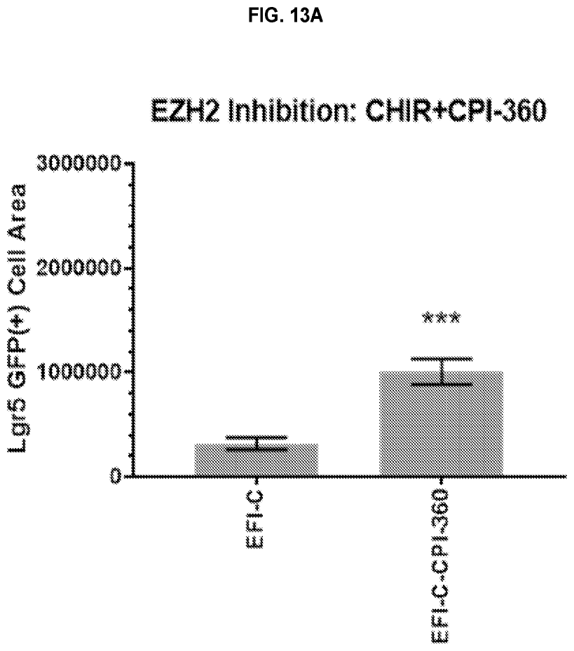

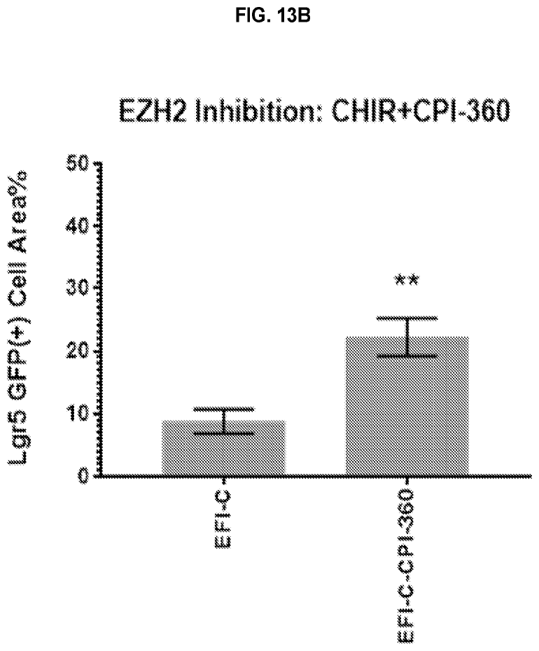

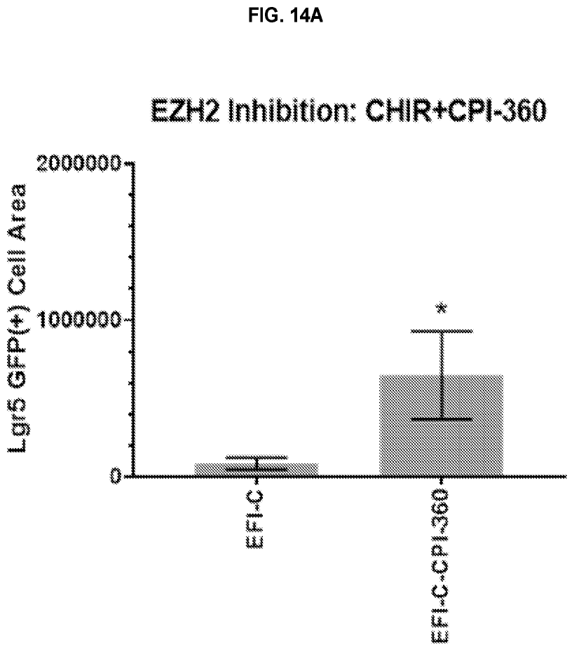

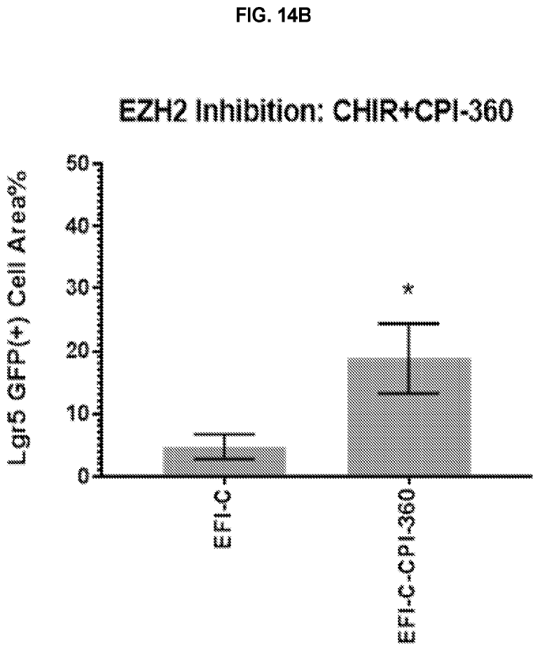

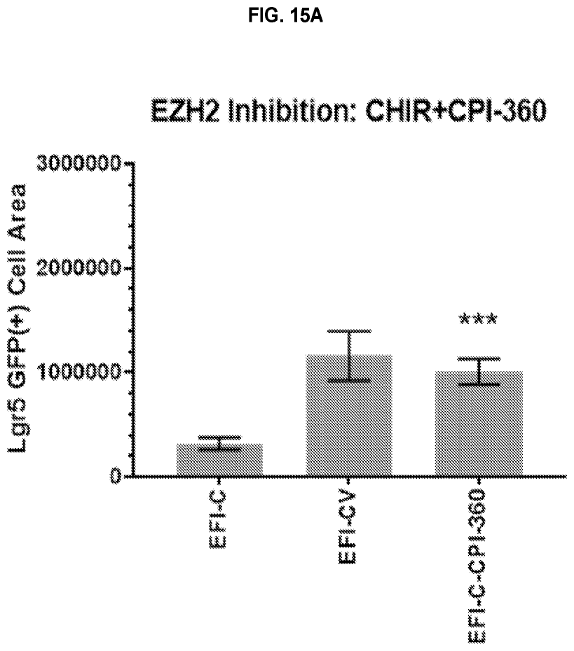

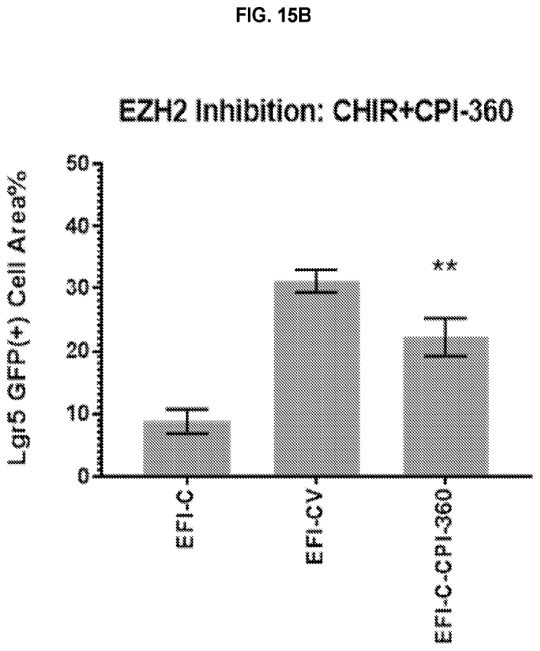

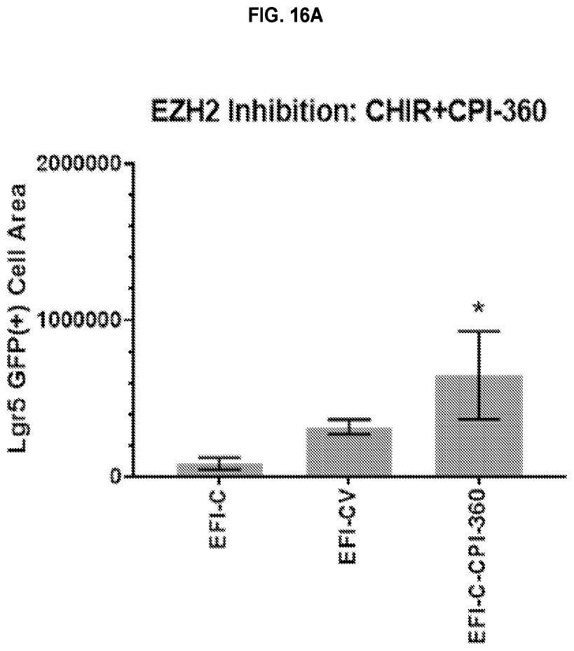

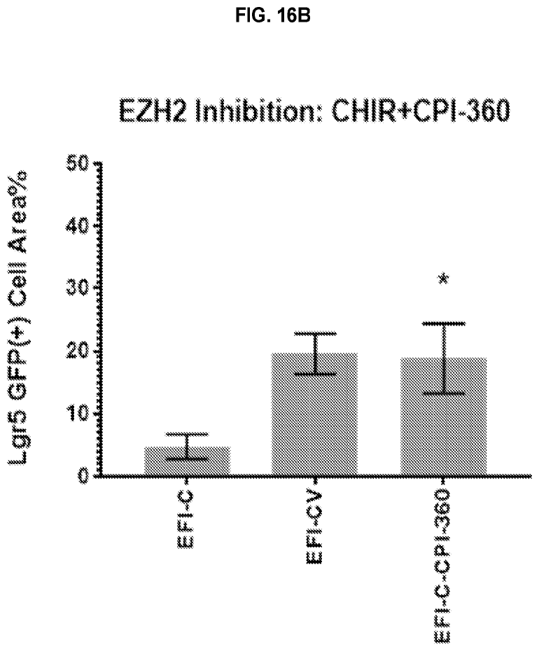

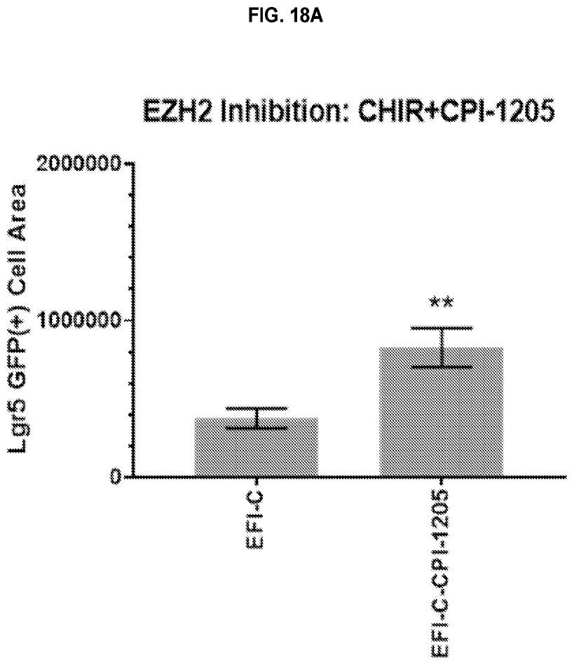

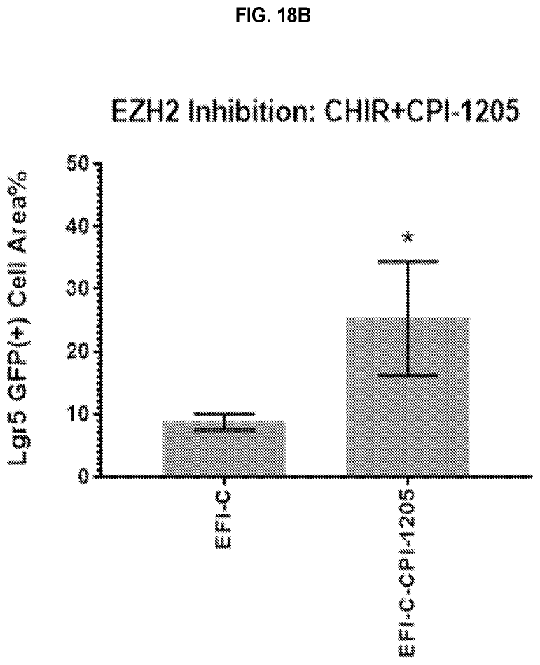

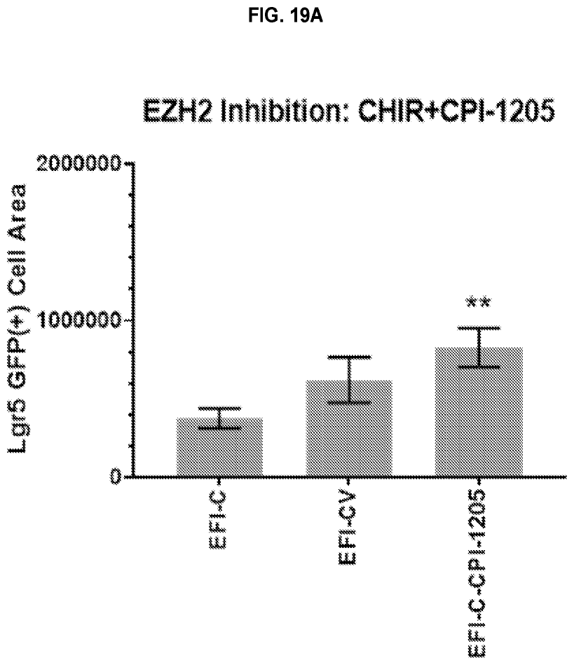

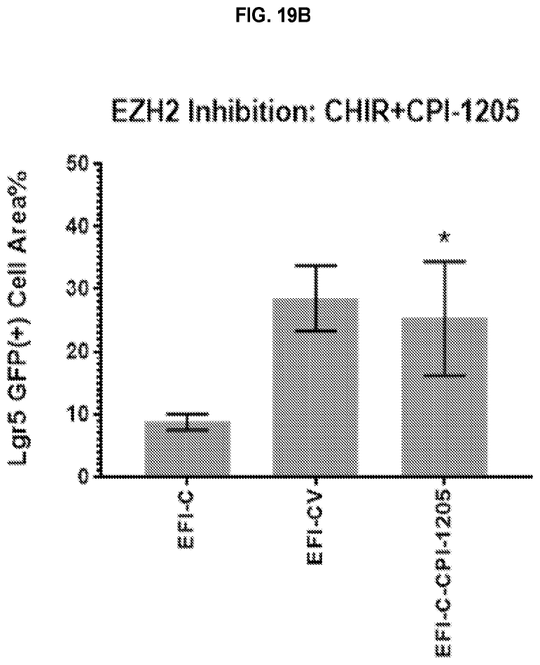

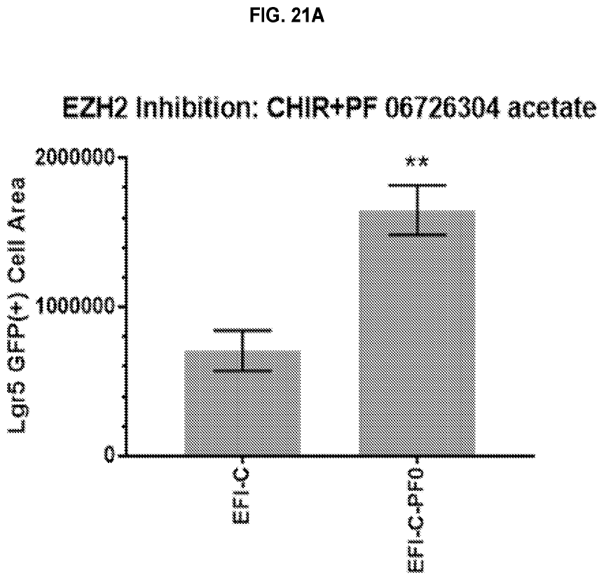

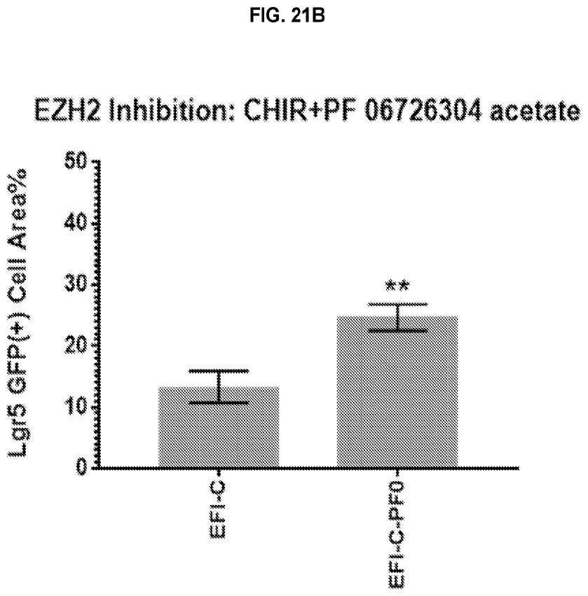

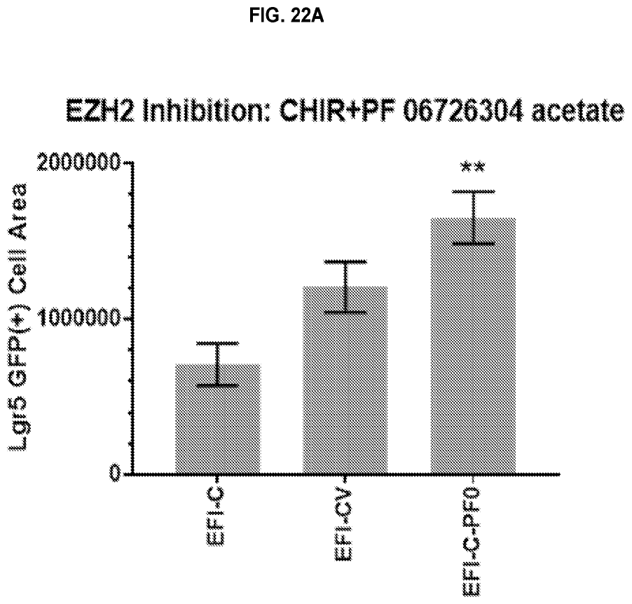

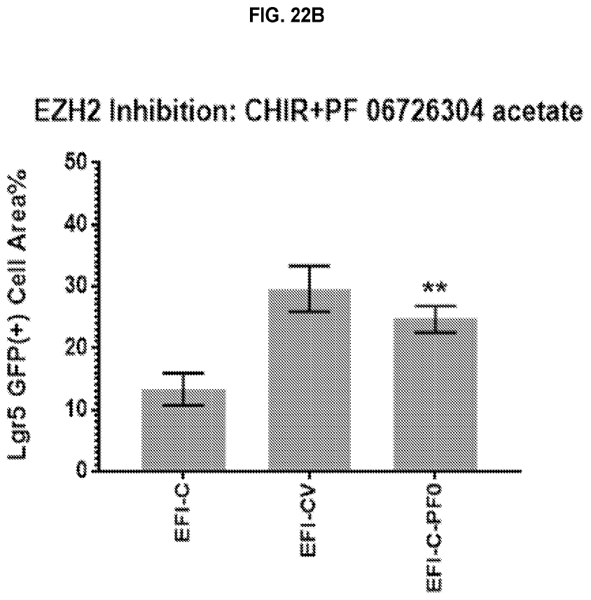

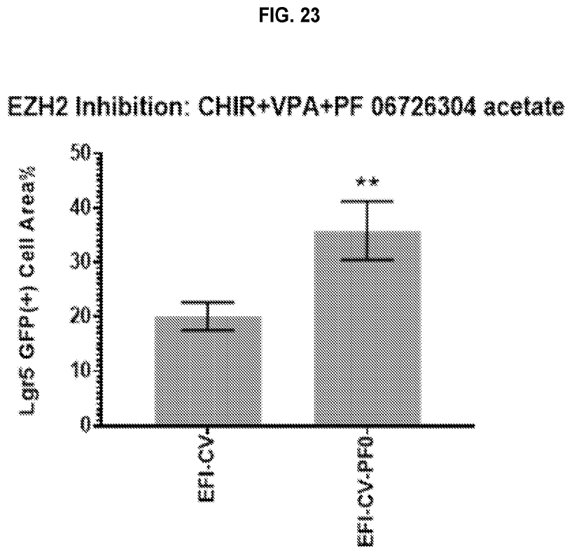

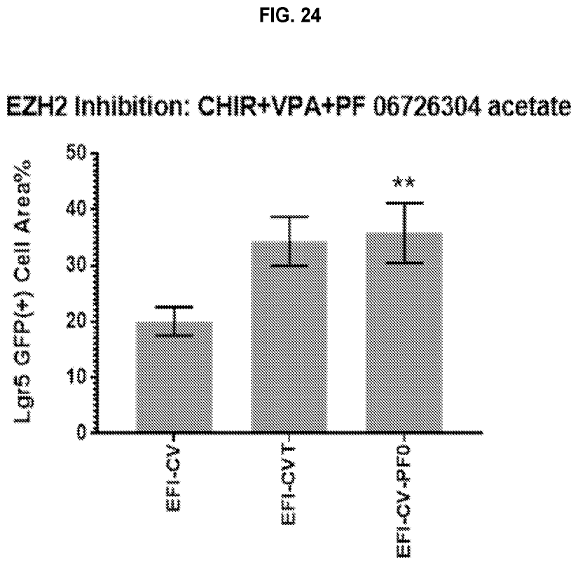

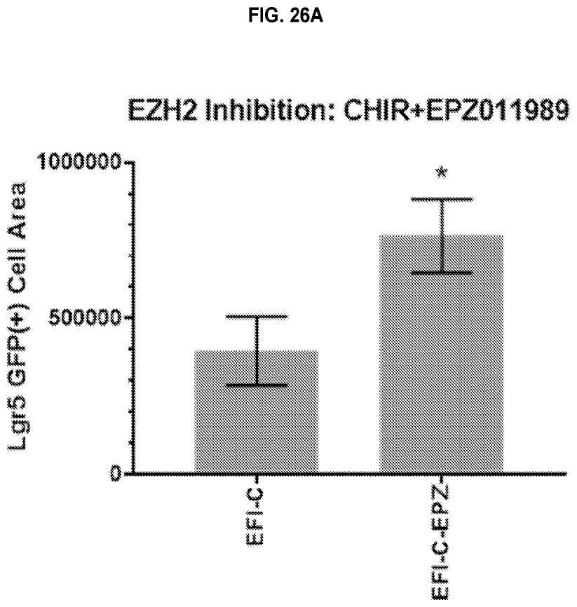

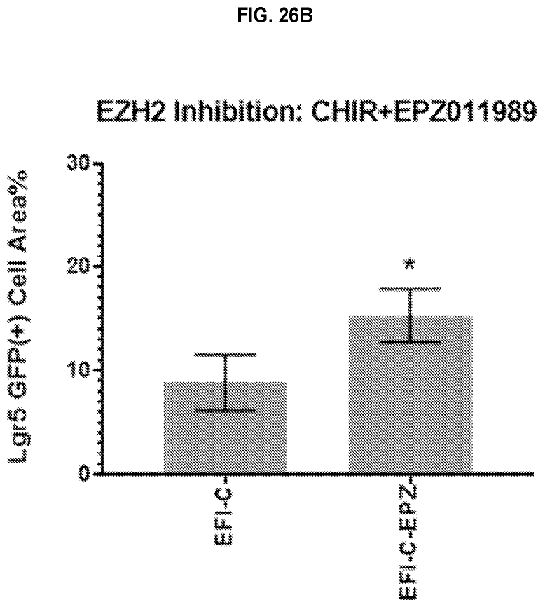

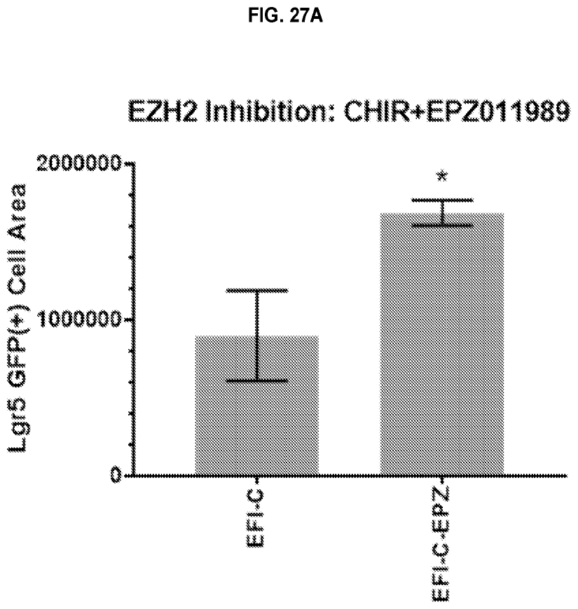

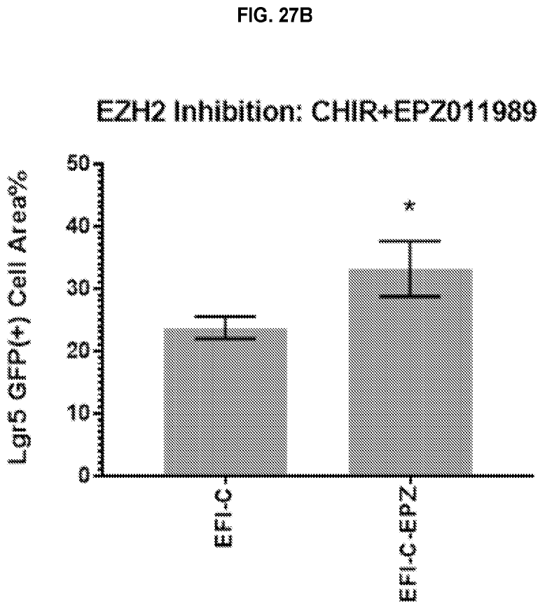

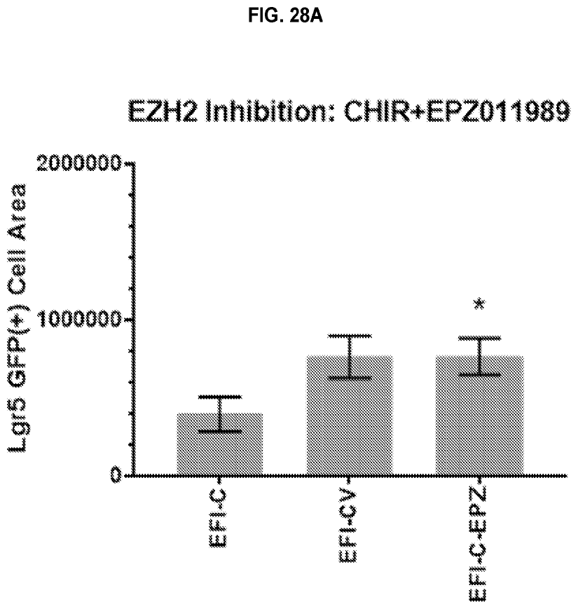

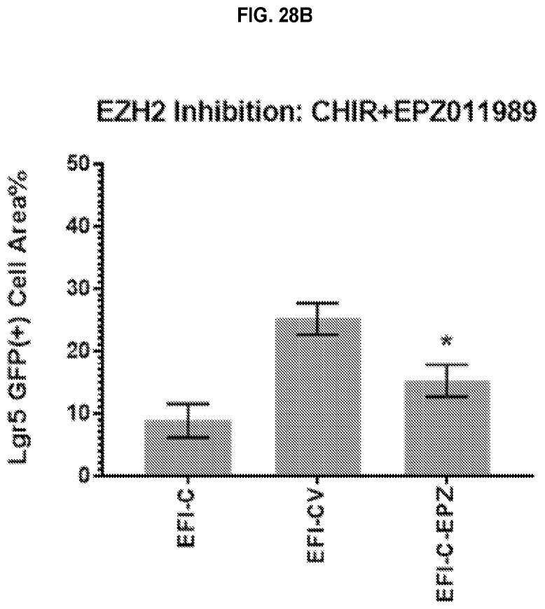

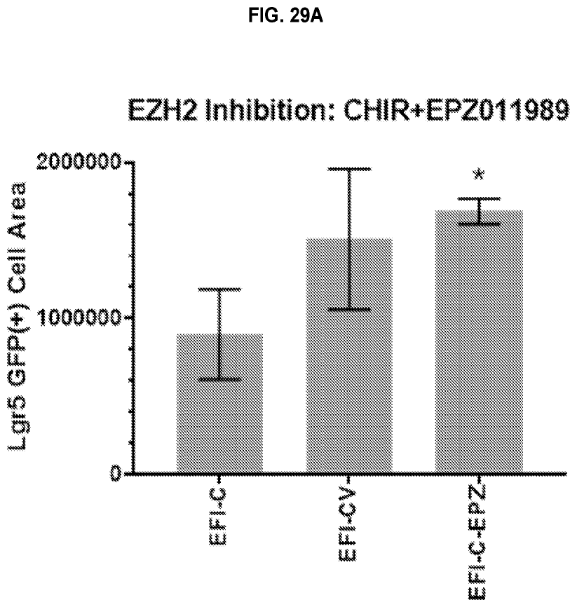

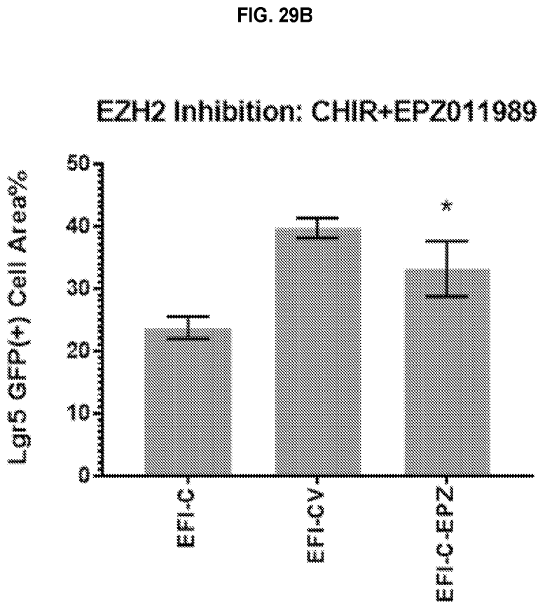

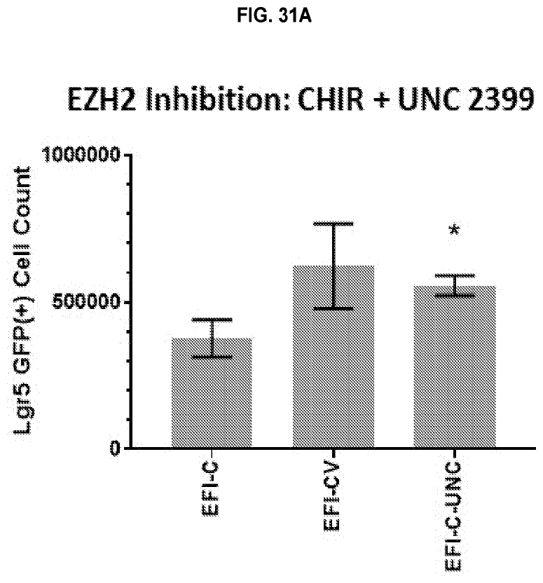

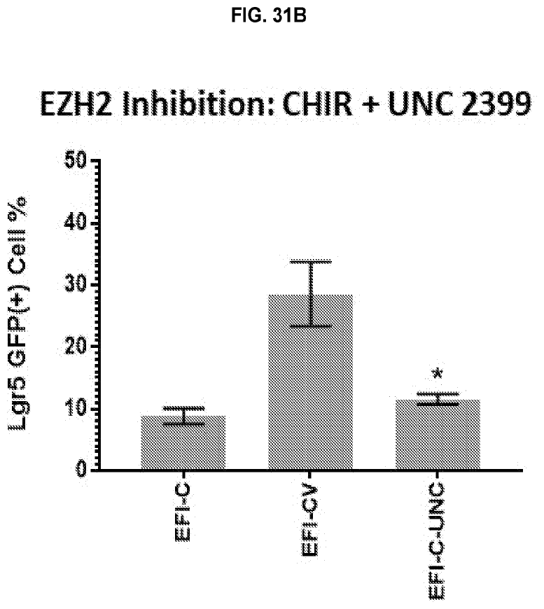

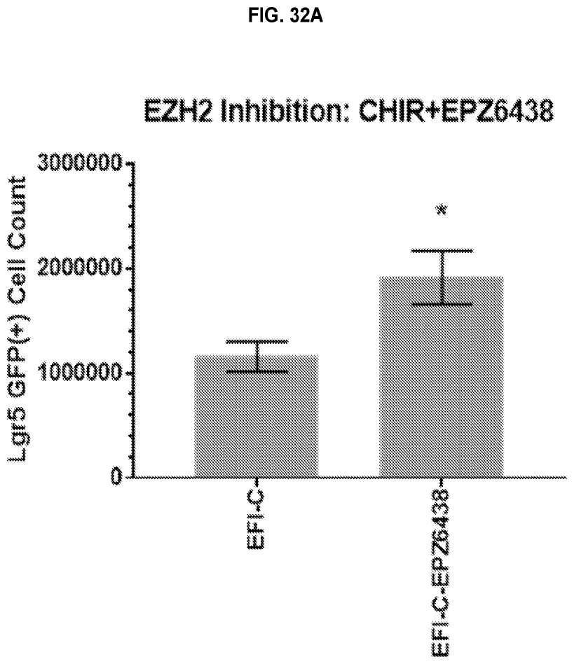

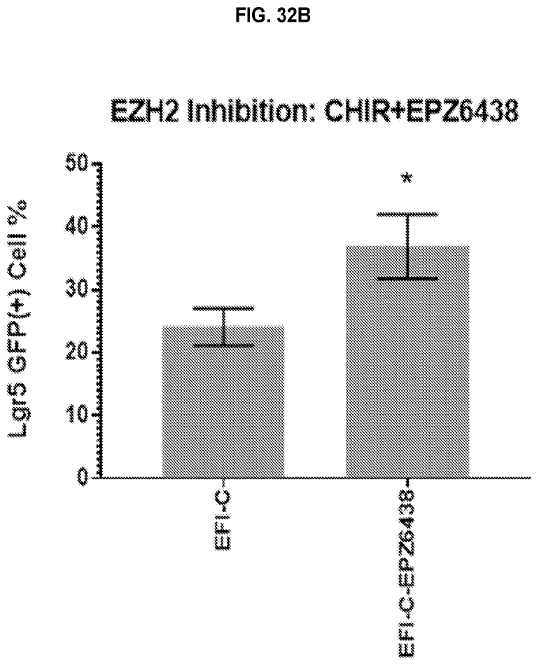

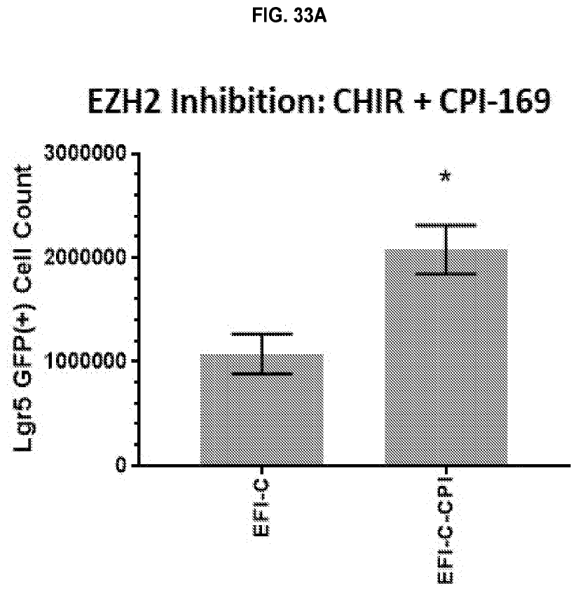

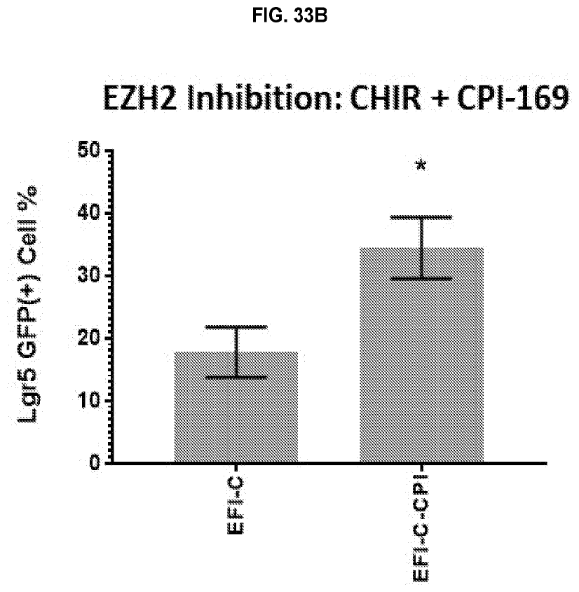

Cellular assays were carried about as described in Example 1 to determine the effect of EZH2 inhibition in combination with a Wnt agonist and VPA on the expansion of cochlear progenitor cells. As shown in FIG. 9A and FIG. 9B, Lgr5+ progenitor cells have increased proliferation and enrichment treated with EL1 in combination with Wnt Agonist, CHIR99021. Moreover, as shown in FIG. 32A, FIG. 32B, the EZH2 inhibitor EPZ6438 in combination with CHIR99021 induced Lgr5+ progenitor cell proliferation and enrichment. The combination of EPZ6438 and CHIR99021 also showed enhanced proliferation and enrichment compared to CHIR99021 and VPA (see FIG. 10A, FIG. 10B). Similar results on Lgr5+ progenitor progenitor cell proliferation and enrichment are achieved with EZH2 inhibitor CPI-169 and CHIR99021 (see FIG. 11A, FIG. 11B, FIG. 33A, FIG. 33B). Proliferation and enrichment of Lgr5+ progenitor cells was also enhanced by EZH2 inhibitor CPI-360 in combination with CHIR99021 (see FIG. 13A, FIG. 13B, FIG. 14A, FIG. 14B, FIG. 15A, FIG. 15B, FIG. 16A, and FIG. 16B). As shown in FIG. 18A, FIG. 18B, FIG. 19A, and FIG. 19B, similar results showing enhanced Lgr5+ progenitor cell proliferation and enrichment are achieved with EZH2 inhibitor CPI-1205 and CHIR99021. As shown in FIG. 21A, FIG. 21B, FIG. 22A, and FIG. 22B, similar results on Lgr5+ progenitor cell proliferation and enrichment are achieved with EZH2 inhibitor PF 06726304 acetate and CHIR99021. As shown in FIG. 23 and FIG. 24 Lgr5+ progenitor cells are enriched following EZH2 inhibitor PF 06726304 acetate and CHIR99021. As shown in FIG. 26A, FIG. 26B, FIG. 27A, FIG. 27B, FIG. 28A, FIG. 28B, FIG. 29A, AND FIG. 29B similar results on Lgr5+ progenitor cell proliferation and enrichment are achieved with EZH2 inhibitor EPZ011989 and CHIR99021. As shown in FIG. 31A, and FIG. 31B, similar results on Lgr5+ progenitor cell proliferation and enrichment are achieved with EZH2 inhibitor UNC 2399 and CHIR99021. Taken together, the results show that inhibition of EZH2 by a panel of EZH2 inhibitors in combination with a Wnt agonist enhances cochlear progenitor cell proliferation and enrichment.

### Example 6: DOT1L Inhibition in Combination with a Wnt Agonist Enhances the Expansion of Cochlear Progenitor Cells

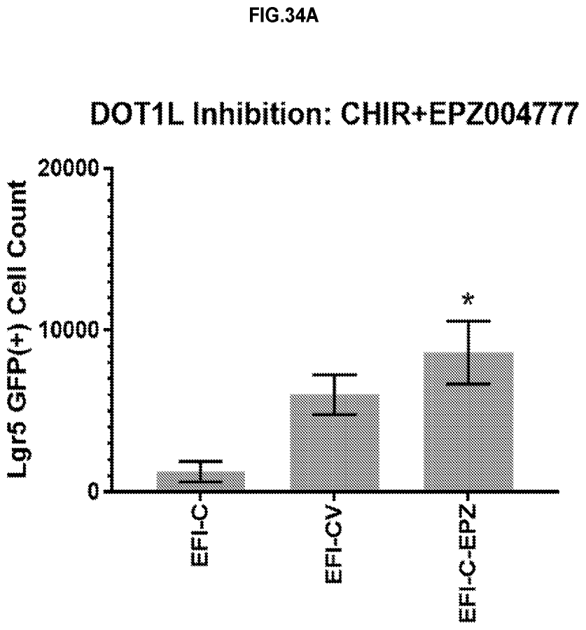

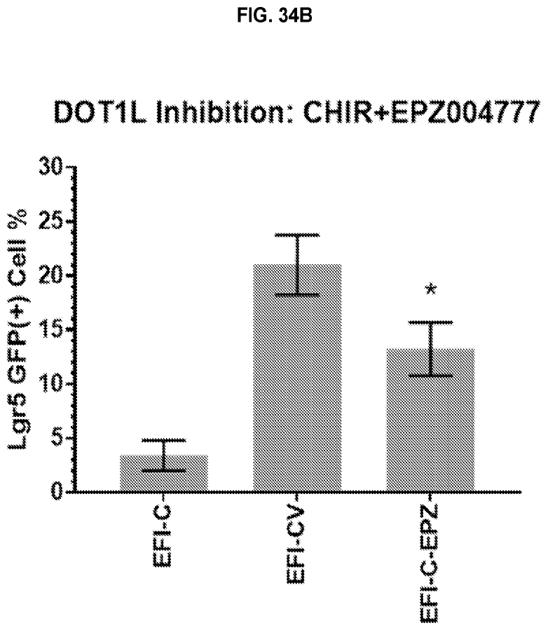

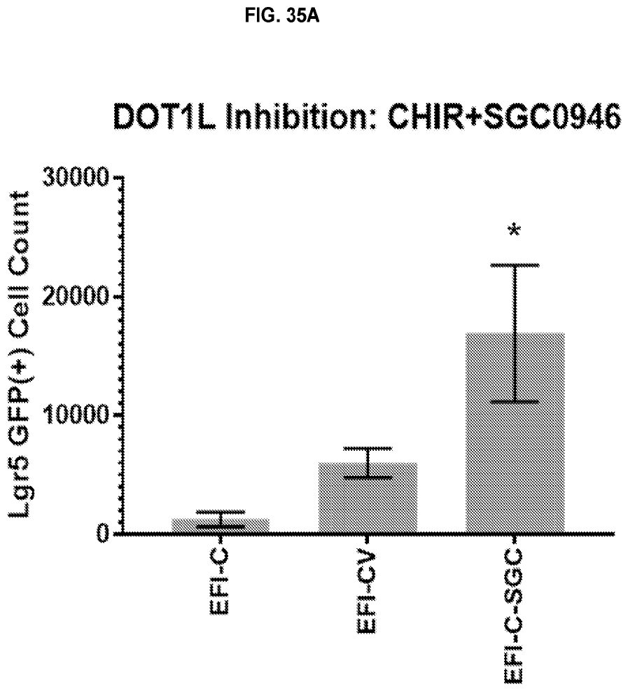

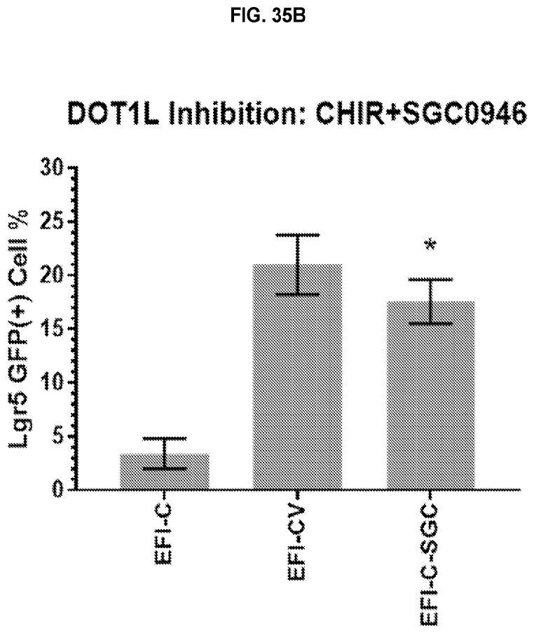

Cellular assays were carried about as described in Example 1 to determine the effect of DOT1L inhibition in combination with a Wnt agonist on the expansion of cochlear progenitor cells. As shown in FIG. 34A and FIG. 34B, Lgr5+ progenitor cells proliferation and enrichment are enhanced when DOT1L inhibitor EPZ004777 is combined with the Wnt Agonist, CHIR99021 compared to CHIR99021 alone. Additionally, as shown in FIG. 35A and FIG. 35B, Lgr5+ progenitor cells proliferation and enrichment are enhanced when DOT1L inhibitor SGC0946 is combined with Wnt Agonist, CHIR99021. Taken together, the results show that inhibition of DOT1L by a panel of DOT1L inhibitors in combination with a Wnt agonist enhances cochlear progenitor cell proliferation and enrichment.

### Example 7: KDM Inhibition in Combination with a Wnt Agonist Enhances the Expansion of Cochlear Progenitor Cells

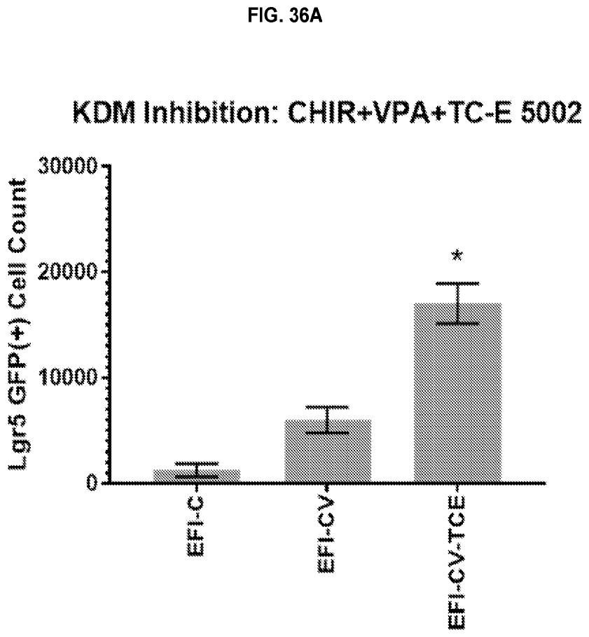

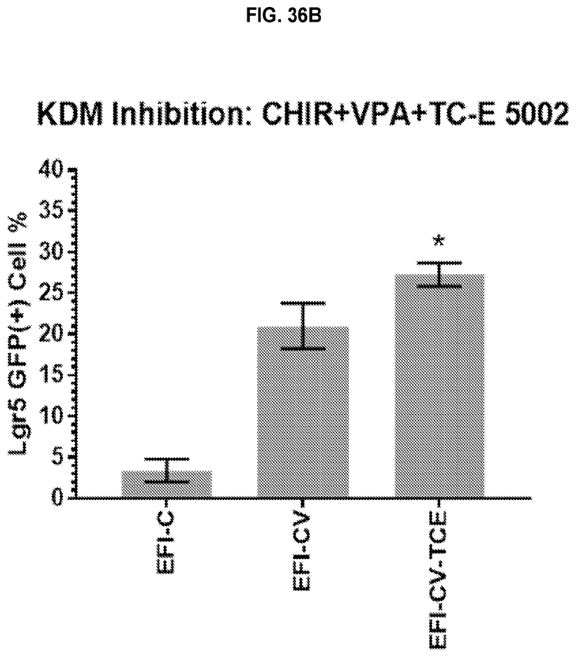

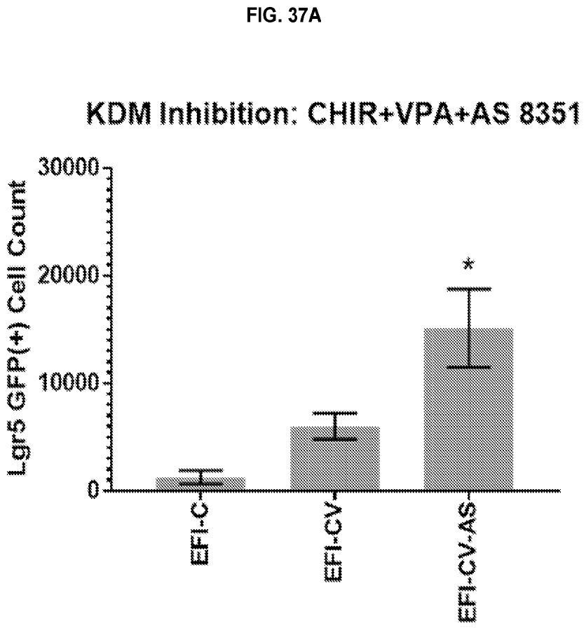

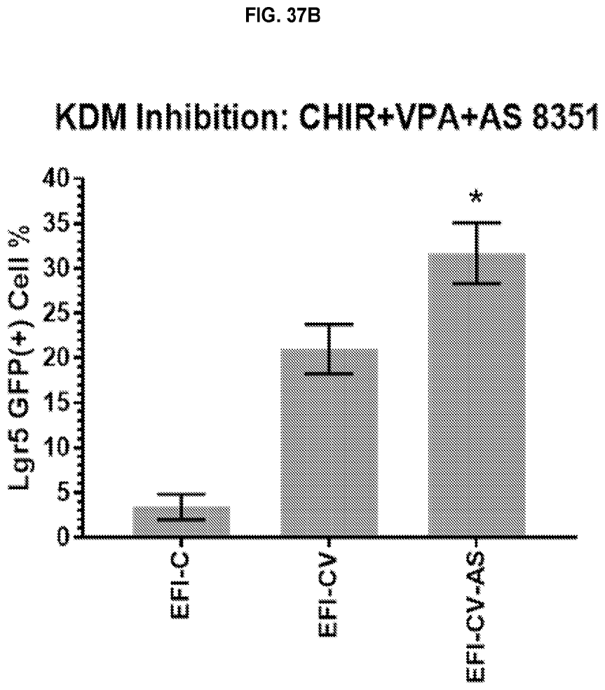

Cellular assays were carried about as described in Example 1 to determine the effect of KDM inhibition in combination with a Wnt agonist on the expansion of cochlear progenitor cells. As shown in FIG. 36A and FIG. 36B, Lgr5+ progenitor cells proliferation and enrichment are enhanced when KDM inhibitor TC-E 5002 is combined with Wnt Agonist, CHIR99021 and VPA when compared to CHIR99021 alone. As shown in FIG. 37A, FIG. 37B, similar results on Lgr5+ progenitor cell proliferation and enrichment are achieved with KDM inhibitor AS 8351 in combination with CHIR99021 and VPA when compared to CHIR99021 alone.

We claim:

1. A method for increasing proliferation of a cochlear supporting cell or a vestibular supporting cell, comprising contacting the supporting cell with:
a) a first epigenetic agent; and
b) a Wnt agonist;
wherein (a) and (b) can occur in any order or simultaneously, thereby increasing cochlear supporting cell or vestibular supporting cell proliferation compared to a vehicle control.

2. A method for producing an expanded population of cochlear or vestibular cells, comprising contacting a population of cochlear supporting cells or vestibular supporting cells with:
a) a first epigenetic agent and;
b) a Wnt agonist
wherein (a) and (b) can occur in any order or simultaneously, thereby producing an expanded population of cochlear or vestibular cells compared to a vehicle control.

3. The method of claim 1 or 2, further comprising cochlear supporting cell or a vestibular supporting cell with: c) a second epigenetic agent wherein (a), (b) or (c) can occur in any order or simultaneously, thereby increasing cochlear supporting cell or vestibular supporting cell proliferation compared to a vehicle control.

4. The method of any preceding claim, wherein
a) the first epigenetic agent epigenetic agent is a lysine specific demethylase 1 (LSD1) inhibitor, an enhancer of zeste homolog 2 (EZH2) inhibitor, a disruptor of telomeric silencing 1-like (DOT1L) inhibitor, or a histone lysine demethylase (KDM) inhibitor; and
b) the second epigenetic agent is an HDAC inhibitor, an LSD1 inhibitor, an EZH2 inhibitor, a DOT1L inhibitor a or KDM inhibitor.

5. The method of any preceding claim, wherein the cochlear supporting cell(s) or vestibular supporting cell(s) express(es) leucine-rich repeat-containing G-protein coupled receptor 5 (Lgr5).

6. The method of any preceding claim, wherein the cochlear supporting cell(s) or vestibular supporting cell(s) are/is a mature cell(s).

7. The method of any preceding claim, wherein the expanded population of cochlear or vestibular cells expresses leucine-rich repeat-containing G-protein coupled receptor 5 (Lgr5).

8. The method of any preceding claim, wherein the epigenetic agent in combination with the Wnt agonist increases the Lgr5 Activity of the expanded population of cochlear or vestibular cells by a factor of at least 10, 20, 30, 40, 50, 75, 100 or 200% compared to a Wnt agonist alone or a Wnt agonist in combination with valproic acid, wherein the Lgr5 Activity is measured in a Stem Cell Proliferation Assay

9. A method of treating a subject who has, or is at risk of, developing an inner ear hearing or balance disorder, comprising administering to the subject:
a) a first epigenetic agent; and
b) a Wnt agonist
wherein (a) and (b) can occur in any order or simultaneously.

10. The method of claim 9, further comprising administering to the subject: c) a second epigenetic agent wherein (a), (b) or (c) can occur in any order or simultaneously.

11. The method of claim 9 or 10, wherein a) the first epigenetic agent epigenetic agent is a lysine specific demethylase 1 (LSD1) inhibitor, an enhancer of zeste homolog 2 (EZH2) inhibitor, a disruptor of telomeric silencing 1-like (DOT1L) inhibitor, or a histone lysine demethylase (KDM) inhibitor; and
b) the second epigenetic agent is an HDAC inhibitor, an LSD1 inhibitor, an EZH2 inhibitor, a DOT1L inhibitor a or KDM inhibitor.

12. The method of any of claims 9-11, wherein the subject has an inner ear hearing or balance disorder.

13. The method of any of claims 12, wherein the inner ear hearing or balance disorder is sensorineural hearing loss.

14. The method of any of claims 9-13, wherein the treatment results in improved auditory function when assessed by behavioural audiometry or auditory brainstem response (ABR) testing.

15. The method of any preceding claim, wherein the epigenetic agent is an LSD1 inhibitor selected from the group consisting of GSK-2879552, GSK-LSD1, Tranylcypromine, Phenelzine sulfate, RN-1, and ORY-1001.

16. The method of any preceding claim, wherein the epigenetic agent is an EZH2 inhibitor selected from the group consisting of: CPI-1205, CPI-169, CPI-360, EPZ011989, E11, PF-06821497, UNC 2399, tazemetostat, valemetostat, and PF 06726304.

17. The method of any preceding claim, wherein the wherein the epigenetic agent is a DOT1L inhibitor selected from the group consisting of EPZ004777, pinometostat and SGC0946.

18. The method of any preceding claim, wherein the wherein the epigenetic agent is KDM inhibitor is selected from the group consisting AS 8351, EPT 103182, and TC-E 5002.

19. The method of any claims 3-18, wherein the second epigenetic is an HDAC inhibitor that is Valproic Acid (VPA)

20. The method of any preceding claim, wherein the Wnt agonist is a GSK3 inhibitor.

21. The method of claim 20, wherein the GSK3 inhibitor is selected from the group consisting of: AZD1080, LY2090314, a substituted 3-Imidazo[1,2-a]pyridin-3-yl-4-(1,2,3,4-tetrahydro-[1,4]diazepino-[6,7,1-hi]indol-7-yl)pyrrole-2,5-dione, GSK3 inhibitor XXII or CHIR99021.

22. The method of any preceding claim, wherein the epigenetic agent is administered locally and/or systemically.

23. The method of any preceding claim, wherein the Wnt agonist is administered locally and/or systemically.

24. The method of any of claims 22-23, wherein the local administration is to the tympanic membrane, the middle ear or the inner ear.

25. The method of claim 24, wherein the local administration is to the middle ear.

26. The method of any of claims 22-23, wherein the systemic administration is oral or parenteral.

27. A pharmaceutical composition comprising a first epigenetic agent, a Wnt agonist, and a pharmaceutically acceptable carrier.

28. The pharmaceutical composition of claim 27, further a second epigenetic agent.

29. The pharmaceutical composition of claim 27 or 28, wherein:
a) the first epigenetic agent epigenetic agent is a lysine specific demethylase 1 (LSD1) inhibitor, an enhancer of zeste homolog 2 (EZH2) inhibitor, a disruptor of telomeric silencing 1-like (DOT1L) inhibitor, or a histone lysine demethylase (KDM) inhibitor; and
b) the second epigenetic agent is an HDAC inhibitor, an LSD1 inhibitor, an EZH2 inhibitor, a DOT1L inhibitor a or KDM inhibitor.

30. The pharmaceutical composition of any of claims 27-29, wherein the epigenetic agent is an LSD1 inhibitor selected from the group consisting of GSK-2879552, GSK-LSD1, RN-1, Tranylcypromine, Phenelzine sulfate, and ORY-1001.

31. The pharmaceutical composition of any of claims 27-29, wherein the epigenetic agent is EZH2 inhibitor selected from the group consisting of: CPI-1205, CPI-169, CPI-360, EPZ011989, E11, PF-06821497, UNC 2399, tazemetostat, valemetostat, PF06726304.

32. The pharmaceutical composition of any of claims 27-29, wherein the wherein the epigenetic agent is a DOT1L inhibitor selected from the group consisting of EPZ004777, pinometostat and SGC0946.

33. The pharmaceutical composition of any of claims 27-29, wherein the wherein the epigenetic agent is KDM inhibitor is selected from the group consisting AS 8351, EPT 103182, and TC-E 5002.

34. The pharmaceutical composition of any of claims 27-29, wherein the second epigenetic is an HDAC inhibitor that is Valproic Acid (VPA).

35. The pharmaceutical composition of any of claims 27-34, wherein the Wnt agonist is a GSK3 inhibitor.

36. The pharmaceutical composition of claim 35, wherein the GSK3 inhibitor is selected from the group consisting of: AZD1080, LY2090314, a substituted 3-Imidazo[1,2-a]pyridin-3-yl-4-(1,2,3,4-tetrahydro-[1,4]diazepino-[6,7,1-hi]indol-7-yl)pyrrole-2,5-dione, GSK3 inhibitor XXII or CHIR99021.

37. The pharmaceutical composition of any of claims 27-36, wherein the pharmaceutical composition is in a biocompatible matrix.

38. The pharmaceutical composition of claim 38, wherein the biocompatible matrix comprises hyaluronic acid, hyaluronates, lecithin gels, pluronics, poly(ethyleneglycol), poloxamers, chitosans, xyloglucans, collagens, fibrins, polyesters, poly(lactides), poly(glycolide), poly(lactic-co-glycolic acid (PLGA), sucrose acetate isobutyrate, glycerol monooleate, poly anhydrides, poly caprolactone sucrose, glycerol monooleate, silk materials, or a combination thereof.

39. The pharmaceutical composition of any of claims 27-38, wherein the pharmaceutical composition is formulated for local or systemic administration.

40. The pharmaceutical composition any of claims 27-39 for use in treating or preventing an inner ear hearing or balance disorder.

41. The pharmaceutical composition for use according to claim 40, wherein the inner ear hearing or balance disorder is sensorineural hearing loss.

42. Use of the pharmaceutical composition of any of claims 27-41 in the manufacture of a medicament for the treatment or prevention of an inner ear hearing or balance disorder.

43. A container comprising an epigenetic agent and instructions, where those instructions describe the epigenetic agent's use for treating or preventing an inner ear hearing or balance disorder in a subject, wherein the instructions require that the subject has been, or will be, administered a Wnt agonist.

44. A container comprising a Wnt agonist and instructions, where those instructions describe the Wnt agonist's use in treating or preventing an inner ear hearing or balance disorder in a subject, wherein the instructions require that the subject has been, or will be, administered an epigenetic agent.

45. A container comprising an epigenetic agent and instructions, where those instructions describe the epigenetic agent's use in treating or preventing an inner ear hearing or balance disorder in a subject, wherein the instructions require that the subject has been, or will be, administered an epigenetic agent and a Wnt agonist.

46. The container according to any of claims 43-45, wherein the inner ear hearing or balance disorder is sensorineural hearing loss.
